SEQUENCE LISTING (920 sequences)

SEQ ID NO: 1 - RNA, 20 nt, synthetic construct
  source 1..20: mol_type = other RNA
atctgtgggacaagaggatc

SEQ ID NO: 2 - RNA, 20 nt, synthetic construct
  source 1..20: mol_type = other RNA
ctggatatctgtgggacaag

SEQ ID NO: 3 - RNA, 20 nt, synthetic construct
  source 1..20: mol_type = other RNA
cttacctgggctggggaaga

SEQ ID NO: 4 - RNA, 20 nt, synthetic construct
  source 1..20: mol_type = other RNA
ttcgtatctgtaaaaccaag

SEQ ID NO: 5 - RNA, 20 nt, synthetic construct
  source 1..20: mol_type = other RNA
tttcaaaacctgtcagtgat

SEQ ID NO: 6 - RNA, 20 nt, synthetic construct
  source 1..20: mol_type = other RNA
ttacctgtaccataaccagg

SEQ ID NO: 7 - RNA, 20 nt, synthetic construct
  source 1..20: mol_type = other RNA
ctcttacctgtaccataacc

SEQ ID NO: 8 - RNA, 20 nt, synthetic construct
  source 1..20: mol_type = other RNA
gtatctgtaggaggagaagt

SEQ ID NO: 9 - RNA, 20 nt, synthetic construct
  source 1..20: mol_type = other RNA
actcacgctggatagcctcc

SEQ ID NO: 10 - RNA, 20 nt, synthetic construct
  source 1..20: mol_type = other RNA
tcgatctatgaaaaagacag

SEQ ID NO: 11 - RNA, 20 nt, synthetic construct
  source 1..20: mol_type = other RNA
caaagattcaggtttactca

SEQ ID NO: 12 - RNA, 20 nt, synthetic construct
  source 1..20: mol_type = other RNA
actttgtcacagcccaagat

SEQ ID NO: 13 - RNA, 20 nt, synthetic construct
  source 1..20: mol_type = other RNA
gtcacagcccaagatagtta

SEQ ID NO: 14 - RNA, 20 nt, synthetic construct
  source 1..20: mol_type = other RNA
cacagcccaagatagttaag

SEQ ID NO: 15 - RNA, 20 nt, synthetic construct
  source 1..20: mol_type = other RNA
aaagaccagtccttgctgaa

SEQ ID NO: 16 - RNA, 20 nt, synthetic construct
  source 1..20: mol_type = other RNA
atagaaagaccagtccttgc

SEQ ID NO: 17 - RNA, 20 nt, synthetic construct
  source 1..20: mol_type = other RNA
ttaccccacttaactatctt

SEQ ID NO: 18 - RNA, 20 nt, synthetic construct
  source 1..20: mol_type = other RNA
cttaccccacttaactatct

SEQ ID NO: 19 - RNA, 20 nt, synthetic construct
  source 1..20: mol_type = other RNA
tggagtacctgaggaatatc

SEQ ID NO: 20 - RNA, 20 nt, synthetic construct
  source 1..20: mol_type = other RNA
tgcagatcccacaggcgccc

SEQ ID NO: 21 - RNA, 20 nt, synthetic construct
  source 1..20: mol_type = other RNA
cgactggccagggcgcctgt

SEQ ID NO: 22 - RNA, 20 nt, synthetic construct
  source 1..20: mol_type = other RNA
accgcccagacgactggcca

SEQ ID NO: 23 - RNA, 20 nt, synthetic construct
  source 1..20: mol_type = other RNA
cggtgctacaactgggctgg

SEQ ID NO: 24 - RNA, 20 nt, synthetic construct
  source 1..20: mol_type = other RNA
cacctacctaagaaccatcc

SEQ ID NO: 25 - RNA, 20 nt, synthetic construct
  source 1..20: mol_type = other RNA
ggagtctgagagatggagag

SEQ ID NO: 26 - RNA, 20 nt, synthetic construct
  source 1..20: mol_type = other RNA
ggggttccagggcctgtctg

SEQ ID NO: 27 - RNA, 20 nt, synthetic construct
  source 1..20: mol_type = other RNA
cagcaaccagacggacaagc

SEQ ID NO: 28 - RNA, 20 nt, synthetic construct
  source 1..20: mol_type = other RNA
ggaccgcagccagcccggcc

SEQ ID NO: 29 - RNA, 20 nt, synthetic construct
  source 1..20: mol_type = other RNA
gtgtcacacaactgcccaac

SEQ ID NO: 30 - RNA, 20 nt, synthetic construct
  source 1..20: mol_type = other RNA
cgcagatcaaagagagcctg

SEQ ID NO: 31 - RNA, 20 nt, synthetic construct
  source 1..20: mol_type = other RNA
ttctctctggaagggcacaa

SEQ ID NO: 32 - RNA, 20 nt, synthetic construct
  source 1..20: mol_type = other RNA
agccggccagttccaaaccc

SEQ ID NO: 33 - RNA, 19 nt, synthetic construct
  source 1..19: mol_type = other RNA
cagttccaaaccctggtgg

SEQ ID NO: 34 - RNA, 20 nt, synthetic construct
  source 1..20: mol_type = other RNA
ggacccagactagcagcacc

SEQ ID NO: 35 - RNA, 20 nt, synthetic construct
  source 1..20: mol_type = other RNA
tccctgcagagaaacacact

SEQ ID NO: 36 - RNA, 20 nt, synthetic construct
  source 1..20: mol_type = other RNA
gagactcaccaggggctggc

SEQ ID NO: 37 - RNA, 20 nt, synthetic construct
  source 1..20: mol_type = other RNA
ttctttgaggagaaagggag

SEQ ID NO: 38 - RNA, 20 nt, synthetic construct
  source 1..20: mol_type = other RNA
cctccttctttgaggagaaa

SEQ ID NO: 39 - RNA, 20 nt, synthetic construct
  source 1..20: mol_type = other RNA
agagtctctcagctggtaca

SEQ ID NO: 40 - RNA, 20 nt, synthetic construct
  source 1..20: mol_type = other RNA
tggatttagagtctctcagc

SEQ ID NO: 41 - RNA, 20 nt, synthetic construct
  source 1..20: mol_type = other RNA
taggcagacagacttgtcac

SEQ ID NO: 42 - RNA, 20 nt, synthetic construct
  source 1..20: mol_type = other RNA
acctggccattcctgaagca

SEQ ID NO: 43 - RNA, 20 nt, synthetic construct
  source 1..20: mol_type = other RNA
ccagagctctgggcagaacc

SEQ ID NO: 44 - RNA, 20 nt, synthetic construct
  source 1..20: mol_type = other RNA
acatcattgaccagagctct

SEQ ID NO: 45 - RNA, 20 nt, synthetic construct
  source 1..20: mol_type = other RNA
ttcggaacccaatcactgac

SEQ ID NO: 46 - RNA, 20 nt, synthetic construct
  source 1..20: mol_type = other RNA
ccactttcaggaggaggatt

SEQ ID NO: 47 - RNA, 20 nt, synthetic construct
  source 1..20: mol_type = other RNA
acccggccactttcaggagg

SEQ ID NO: 48 - RNA, 20 nt, synthetic construct
  source 1..20: mol_type = other RNA
ccataaccgctgtggcctct

SEQ ID NO: 49 - RNA, 20 nt, synthetic construct
  source 1..20: mol_type = other RNA
atgcaagcccataaccgctg

SEQ ID NO: 50 - RNA, 20 nt, synthetic construct
  source 1..20: mol_type = other RNA
cctgcaggccatgcaggcct

SEQ ID NO: 51 - RNA, 20 nt, synthetic construct
  source 1..20: mol_type = other RNA
caagcaactttggggagtcc

SEQ ID NO: 52 - RNA, 20 nt, synthetic construct
  source 1..20: mol_type = other RNA
ctccatgccaagcaactttg

SEQ ID NO: 53 - RNA, 20 nt, synthetic construct
  source 1..20: mol_type = other RNA
ccctccatgccaagcaactt

SEQ ID NO: 54 - RNA, 20 nt, synthetic construct
  source 1..20: mol_type = other RNA
tgaggggctgctggtttggc

SEQ ID NO: 55 - RNA, 20 nt, synthetic construct
  source 1..20: mol_type = other RNA
gctggatgctgaggggctgc

SEQ ID NO: 56 - RNA, 20 nt, synthetic construct
  source 1..20: mol_type = other RNA
ttatgttgctggatgctgag

SEQ ID NO: 57 - RNA, 20 nt, synthetic construct
  source 1..20: mol_type = other RNA
gaggaaggaccagagcggga

SEQ ID NO: 58 - RNA, 20 nt, synthetic construct
  source 1..20: mol_type = other RNA
gggagaggaaggaccagagc

SEQ ID NO: 59 - RNA, 20 nt, synthetic construct
  source 1..20: mol_type = other RNA
gggtgcagagcgggagagga

SEQ ID NO: 60 - RNA, 20 nt, synthetic construct
  source 1..20: mol_type = other RNA
gggccacagagggtgcagag

SEQ ID NO: 61 - RNA, 20 nt, synthetic construct
  source 1..20: mol_type = other RNA
cagagggtgcagagcgggag

SEQ ID NO: 62 - RNA, 20 nt, synthetic construct
  source 1..20: mol_type = other RNA
gaaacaaaaacattttctca

SEQ ID NO: 63 - RNA, 20 nt, synthetic construct
  source 1..20: mol_type = other RNA
tcttttcccgatattcctca

SEQ ID NO: 64 - RNA, 20 nt, synthetic construct
  source 1..20: mol_type = other RNA
aatattaatgtgtcttttcc

SEQ ID NO: 65 - RNA, 20 nt, synthetic construct
  source 1..20: mol_type = other RNA
ataccctggcaatattaatg

SEQ ID NO: 66 - RNA, 20 nt, synthetic construct
  source 1..20: mol_type = other RNA
agccccaagtgaaataccct

SEQ ID NO: 67 - RNA, 20 nt, synthetic construct
  source 1..20: mol_type = other RNA
tatgcctgccgtgtgaacca

SEQ ID NO: 68 - RNA, 20 nt, synthetic construct
  source 1..20: mol_type = other RNA
agatgagtatgcctgccgtg

SEQ ID NO: 69 - RNA, 20 nt, synthetic construct
  source 1..20: mol_type = other RNA
ctgaaaaagatgagtatgcc

SEQ ID NO: 70 - RNA, 20 nt, synthetic construct
  source 1..20: mol_type = other RNA
cacccccactgaaaaagatg

SEQ ID NO: 71 - RNA, 20 nt, synthetic construct
  source 1..20: mol_type = other RNA
tacactgaattcacccccac

SEQ ID NO: 72 - RNA, 20 nt, synthetic construct
  source 1..20: mol_type = other RNA
ggtgaattcagtgtagtaca

SEQ ID NO: 73 - RNA, 20 nt, synthetic construct
  source 1..20: mol_type = other RNA
agtgggggtgaattcagtgt

SEQ ID NO: 74 - RNA, 20 nt, synthetic construct
  source 1..20: mol_type = other RNA
ttttcagtgggggtgaattc

SEQ ID NO: 75 - RNA, 20 nt, synthetic construct
  source 1..20: mol_type = other RNA
ctcatctttttcagtggggg

SEQ ID NO: 76 - RNA, 20 nt, synthetic construct
  source 1..20: mol_type = other RNA
caggcatactcatctttttc

SEQ ID NO: 77 - RNA, 20 nt, synthetic construct
  source 1..20: mol_type = other RNA
ttcagcagcttacaaaagaa

SEQ ID NO: 78 - RNA, 20 nt, synthetic construct
  source 1..20: mol_type = other RNA
aactttcagcagcttacaaa

SEQ ID NO: 79 - RNA, 20 nt, synthetic construct
  source 1..20: mol_type = other RNA
actcatacacaactttcagc

SEQ ID NO: 80 - RNA, 20 nt, synthetic construct
  source 1..20: mol_type = other RNA
actactcatacacaactttc

SEQ ID NO: 81 - RNA, 20 nt, synthetic construct
  source 1..20: mol_type = other RNA
tttgactttccattctctgc

SEQ ID NO: 82 - RNA, 20 nt, synthetic construct
  source 1..20: mol_type = other RNA
acccagacacatagcaattc

SEQ ID NO: 83 - RNA, 20 nt, synthetic construct
  source 1..20: mol_type = other RNA
cagtggagaaggcggcactc

SEQ ID NO: 84 - RNA, 20 nt, synthetic construct
  source 1..20: mol_type = other RNA
cgcctgagcagtggagaagg

SEQ ID NO: 85 - RNA, 20 nt, synthetic construct
  source 1..20: mol_type = other RNA
ctccgcctgagcagtggaga

SEQ ID NO: 86 - RNA, 20 nt, synthetic construct
  source 1..20: mol_type = other RNA
gctcacctccgcctgagcag

SEQ ID NO: 87 - RNA, 20 nt, synthetic construct
  source 1..20: mol_type = other RNA
cacctacctaagaaccatcc

SEQ ID NO: 88 - RNA, 20 nt, synthetic construct
  source 1..20: mol_type = other RNA
tgcagatcccacaggcgccc

SEQ ID NO: 89 - RNA, 20 nt, synthetic construct
  source 1..20: mol_type = other RNA
tccaggcatgcagatcccac

SEQ ID NO: 90 - RNA, 20 nt, synthetic construct
  source 1..20: mol_type = other RNA
cactctggtggggctgctcc

SEQ ID NO: 91 - RNA, 20 nt, synthetic construct
  source 1..20: mol_type = other RNA
ccctccagacccctggctct

SEQ ID NO: 92 - RNA, 20 nt, synthetic construct
  source 1..20: mol_type = other RNA
ggaaggtccctccagacccc

SEQ ID NO: 93 - RNA, 20 nt, synthetic construct
  source 1..20: mol_type = other RNA
tgccagggactgagggtgga

SEQ ID NO: 94 - RNA, 20 nt, synthetic construct
  source 1..20: mol_type = other RNA
agcagggctggggagaaggt

SEQ ID NO: 95 - RNA, 20 nt, synthetic construct
  source 1..20: mol_type = other RNA
cacgagcagggctggggaga

SEQ ID NO: 96 - RNA, 20 nt, synthetic construct
  source 1..20: mol_type = other RNA
cccttcggtcaccacgagca

SEQ ID NO: 97 - RNA, 20 nt, synthetic construct
  source 1..20: mol_type = other RNA
ggagaagctgcaggtgaagg

SEQ ID NO: 98 - RNA, 20 nt, synthetic construct
  source 1..20: mol_type = other RNA
gaaggtggcgttgtcccctt

SEQ ID NO: 99 - RNA, 20 nt, synthetic construct
  source 1..20: mol_type = other RNA
cggagagcttcgtgctaaac

SEQ ID NO: 100 - RNA, 20 nt, synthetic construct
  source 1..20: mol_type = other RNA
ctgcagcttctccaacacat

SEQ ID NO: 101 - RNA, 20 nt, synthetic construct
  source 1..20: mol_type = other RNA
catgagccccagcaaccaga

SEQ ID NO: 102 - RNA, 20 nt, synthetic construct
  source 1..20: mol_type = other RNA
cagcaaccagacggacaagc

SEQ ID NO: 103 - RNA, 20 nt, synthetic construct
  source 1..20: mol_type = other RNA
ggaccgcagccagcccggcc

SEQ ID NO: 104 - RNA, 20 nt, synthetic construct
  source 1..20: mol_type = other RNA
cccgaggaccgcagccagcc

SEQ ID NO: 105 - RNA, 20 nt, synthetic construct
  source 1..20: mol_type = other RNA
caagctggccgccttccccg

SEQ ID NO: 106 - RNA, 20 nt, synthetic construct
  source 1..20: mol_type = other RNA
acagcggcacctacctctgt

SEQ ID NO: 107 - RNA, 20 nt, synthetic construct
  source 1..20: mol_type = other RNA
agggcccggcgcaatgacag

SEQ ID NO: 108 - RNA, 20 nt, synthetic construct
  source 1..20: mol_type = other RNA
acatgagcgtggtcagggcc

SEQ ID NO: 109 - RNA, 20 nt, synthetic construct
  source 1..20: mol_type = other RNA
cctgggtgaggggctggggt

SEQ ID NO: 110 - RNA, 20 nt, synthetic construct
  source 1..20: mol_type = other RNA
ggctggcctgggtgaggggc

SEQ ID NO: 111 - RNA, 20 nt, synthetic construct
  source 1..20: mol_type = other RNA
tggccggctggcctgggtga

SEQ ID NO: 112 - RNA, 20 nt, synthetic construct
  source 1..20: mol_type = other RNA
ttgtgcccttccagagagaa

SEQ ID NO: 113 - RNA, 20 nt, synthetic construct
  source 1..20: mol_type = other RNA
tgacgttacctcgtgcggcc

SEQ ID NO: 114 - RNA, 20 nt, synthetic construct
  source 1..20: mol_type = other RNA
tgggatgacgttacctcgtg

SEQ ID NO: 115 - RNA, 20 nt, synthetic construct
  source 1..20: mol_type = other RNA
gcagggcaggccgaggggct

SEQ ID NO: 116 - RNA, 20 nt, synthetic construct
  source 1..20: mol_type = other RNA
gagactcaccaggggctggc

SEQ ID NO: 117 - RNA, 20 nt, synthetic construct
  source 1..20: mol_type = other RNA
gagtgagactcaccaggggc

SEQ ID NO: 118 - RNA, 20 nt, synthetic construct
  source 1..20: mol_type = other RNA
gaaaagagtgagactcacca

SEQ ID NO: 119 - RNA, 20 nt, synthetic construct
  source 1..20: mol_type = other RNA
acccaggaaggaaggcacag

SEQ ID NO: 120 - RNA, 20 nt, synthetic construct
  source 1..20: mol_type = other RNA
ctctgcccacccaggaagga

SEQ ID NO: 121 - RNA, 20 nt, synthetic construct
  source 1..20: mol_type = other RNA
ccacctctgcccacccagga

SEQ ID NO: 122 - RNA, 20 nt, synthetic construct
  source 1..20: mol_type = other RNA
ccatagtccacagagaacac

SEQ ID NO: 123 - RNA, 20 nt, synthetic construct
  source 1..20: mol_type = other RNA
ccggggtcttctctcgccac

SEQ ID NO: 124 - protein, 237 aa, Tobacco etch virus
MGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQTKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 125 - protein, 119 aa, Tobacco etch virus
MGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 126 - protein, 116 aa, Tobacco etch virus
MKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQ

SEQ ID NO: 127 - protein, 7 aa, Tobacco etch virus
ENLYFQS

SEQ ID NO: 128 - protein, 246 aa, Turnip mosaic virus
MASSNSMFRGLRDYNPISNNICHLTNVSDGASNSLYGVGFGPLILTNRHLFERNNGELVIKSRHGEFVIKNTTQLHLLPIPDRDLLLIRLPKDVPPFPQKLGFRQPEKGERICMVGSNFQTKSITSIVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPSGLFKVSKLISDLDSTAVYAQ

SEQ ID NO: 129 - protein, 7 aa, synthetic construct
GGCSHQS

SEQ ID NO: 130 - protein, 246 aa, Plum pox virus
MASSKSLFRGLRDYNPIASSICQLNNSSGARQSEMFGLGFGGLIVTNQHLFKRNDGELTIRSHHGEFVVKDTKTLKLLPCKGRDIVIIRLPKDFPPFPRRLQFRTPTTEDRVCLIGSNFQTKSISSTMSETSATYPVDNSHFWKHWISTKDGHCGLPIVSTRDGSILGLHSLANSTNTQNFYAAFPDNFETTYLSNQDNDNWIKQWRYNPDEVCWGSLQLKRDIPQSPFTICKLLTDLDGEFVYTQ

SEQ ID NO: 131 - protein, 8 aa, synthetic construct
QVVVHQSK

SEQ ID NO: 132 - protein, 247 aa, Potato virus Y
MASAKSLMRGLRDFNPIAQTVCRLKVSVEYGASEMYGFGFGAYIVANHHLFRSYNGSMEVQSMHGTFRVKNLHSLSVLPIKGRDIILIKMPKDFPVFPQKLHFRAPTQNERICLVGTNFQEKYASSIITETSTTYNIPGSTFWKHWIETDNGHCGLPVVSTADGCIVGIHSLANNAHTTNYYSAFDEDFESKYLRTNEHNEWVKSWVYNPDTVLWGPLKLKDSTPKGLFKTTKLVQDLIDHDVVVEQ

SEQ ID NO: 133 - protein, 8 aa, synthetic construct
YDVRHQSR

SEQ ID NO: 134 - protein, 239 aa, Zika virus
MASDMYIERAGDITWEKDAEVTGNSPRLDVALDESGDFSLVEEDGPPMREGGGGSGGGGSGALWDVPAPKEVKKGETTDGVYRVMTRRLLGSTQVGVGVMQEGVFHTMWHVTKGAALRSGEGRLDPYWGDVKQDLVSYCGPWKLDAAWDGLSEVQLLAVPPGERARNIQTLPGIFKTKDGDIGAVALDYPAGTSGSPILDKCGRVIGLYGNGVVIKNGSYVSAITQGKREEETPVECFE

SEQ ID NO: 135 - protein, 8 aa, synthetic construct
KERKRRGA

SEQ ID NO: 136 - protein, 243 aa, West Nile virus
MASSTDMWIERTADISWESDAEITGSSERVDVRLDDDGNFQLMNDPGAPWKGGGGSGGGGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVQTKPGVFKTPEGEIGAVTLDFPTGTSGSPIVDKNGDVIGLYGNGVIMPNGSYISAIVQGERMDEPIPAGFEPEML

SEQ ID NO: 137 - protein, 8 aa, synthetic construct
KQKKRGGK

SEQ ID NO: 138 - protein, 22 aa, Teschovirus A
  source 1..22: note = Porcine teschovirus
GSGATNFSLLKQAGDVEENPGP

SEQ ID NO: 139 - protein, 21 aa, Thosea asigna virus
GSGEGRGSLLTCGDVEENPGP

SEQ ID NO: 140 - protein, 23 aa, Equine rhinitis A virus
GSGQCTNYALLKLAGDVESNPGP

SEQ ID NO: 141 - protein, 223 aa, Mus musculus
MSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 142 - protein, 166 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQ

SEQ ID NO: 143 - protein, 207 aa, Mus musculus
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSS

SEQ ID NO: 144 - protein, 217 aa, Homo sapiens
MQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 145 - protein, 83 aa, Bacillus subtilis
  source 1..83: note = Bacillus subtilis bacteriophage PBS1
TNLSDIIEKETGKQLVIQESILMLPEEVEEVIGNKPESDILVHTAYDESTDENVMLLTSDAPEYKPWALVIQDSNGENKIKML

SEQ ID NO: 146 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 147 - RNA, 109 nt, synthetic construct
  source 1..109: mol_type = other RNA
gtttgagagctaggccaacatgaggatcacccatgtctgcagggcctagcaagttcaaataaggctagtccgttatcaacttgaaaaagtggcaccgagtcggtgcttt

SEQ ID NO: 148 - RNA, 136 nt, synthetic construct
  source 1..136: mol_type = other RNA
gtttgagagctaggccaacatgaggatcacccatgtctgcagggcctagcaagttcaaataaggctagtccgttatcaacttggccaacatgaggatcacccatgtctgcagggccaagtggcaccgagtcggtgc

SEQ ID NO: 149 - RNA, 104 nt, synthetic construct
  source 1..104: mol_type = other RNA
gtttgagagctaccggagcagacgatatggcgtcgctccggtagcaagttcaaataaggctagtccgttatcaacttgaaaaagtggcaccgagtcggtgcttt

SEQ ID NO: 150 - RNA, 122 nt, synthetic construct
  source 1..122: mol_type = other RNA
gtttgagagctaccggagcagacgatatggcgtcgctccggtagcaagttcaaataaggctagtccgttatcaacttggagcagacgatatggcgtcgctccaagtggcaccgagtcggtgc

SEQ ID NO: 151 - RNA, 94 nt, synthetic construct
  source 1..94: mol_type = other RNA
gtttgagagctagggccctgaagaagggccctagcaagttcaaataaggctagtccgttatcaacttgaaaaagtggcaccgagtcggtgcttt

SEQ ID NO: 152 - RNA, 106 nt, synthetic construct
  source 1..106: mol_type = other RNA
gtttgagagctagggccctgaagaagggccctagcaagttcaaataaggctagtccgttatcaacttgggccctgaagaagggcccaagtggcaccgagtcggtgc

SEQ ID NO: 153 - protein, 117 aa, unidentified
  source 1..117: note = Bacteriophage MS2
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 154 - protein, 123 aa, unidentified
  source 1..123: note = Bacteriophage PP7
MGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 155 - protein, 23 aa, synthetic construct
MGNARTRRRERRAEKQAQWKAAN

SEQ ID NO: 156 - protein, 74 aa, Mus musculus
SEKGKQHAEILFLDKIRSMELSQVTITCYLTWSPCPNCAWQLAAFKRDRPDLILHIYTSRLYFHWKRPFQKGLC

SEQ ID NO: 157 - protein, 74 aa, Mus spicilegus
SEKGKQHAEILFLDKIRSMELSQVTITCYLTWSPCPNCAWQLAAFKRDRPDLIPHIYTSRLYFHWKRPFQKGLC

SEQ ID NO: 158 - protein, 74 aa, Cricetulus longicaudatus
SEKGKQHAEILFLDKIRSMELSQVTITCYLTWSPCPNCAWRLAAFKRDRPDLILHIYTSRLYFHWKRPFQKGLC

SEQ ID NO: 159 - protein, 74 aa, Mus terricolor
SEKGKQHAEILFLNKIRSMELSQVTITCYLTWSPCPNCAWQLAAFKKDRPDLILHIYTSRLYFHWKRPFQKGLC

SEQ ID NO: 160 - protein, 74 aa, Mus caroli
SKKGKQHAEILFLDKIRSMELSQVTITCYLTWSPCPNCAWQLAAFKRDHPDLILHIYTSRLYFHWKRPFQKGLC

SEQ ID NO: 161 - protein, 74 aa, Mus pahari
SKKGKQHAEILFLEKIRSMELSQMRITCYLTWSPCPNCAWQLAAFQKDRPDLILHIYTSRLYFHWRRIFQKGLC

SEQ ID NO: 162 - protein, 74 aa, Mus shortridgei; identical to SEQ ID NO: 161

SEQ ID NO: 163 - protein, 74 aa, Mus setulosus
SKKGKQHAEILFLDKIRSMELSQVRITCYLTWSPCPNCAWQLETFKKDRPDLILHIYTSRLYFHWKRAFQEGLC

SEQ ID NO: 164 - protein, 75 aa, Grammomys sp.
  source 1..75: note = Grammomys surdaster
SKKGKPHAEILFLDKMWSMEELSQVRITCYLTWSPCPNCARQLAAFKKDHPGLILRIYTSRLYFYWRRKFQKGLC

SEQ ID NO: 165 - protein, 73 aa, Rattus norvegicus
KKGEQHVEILFLEKMRSMELSQVRITCYLTWSPCPNCARQLAAFKKDHPDLILRIYTSRLYFYWRKKFQKGLC

SEQ ID NO: 166 - protein, 74 aa, Mastomys coucha
SKKGRQHAEILFLEKVRSMQLSQVRITCYLTWSPCPNCAWQLAAFKMDHPDLILRIYASRLYFHWRRAFQKGLC

SEQ ID NO: 167 - protein, 73 aa, Cricetulus barabensis
  source 1..73: note = Cricetulus griseus
NKKGKHAEILFIDEMRSLELGQVQITCYLTWSPCPNCAQELAAFKSDHPDLVLRIYTSRLYFHWRRKYQEGLC

SEQ ID NO: 168 - protein, 73 aa, Peromyscus leucopus
NKKGKHAEILFIDEMRSLELGQARITCYLTWSPCPNCAQKLAAFKKDHPDLVLRVYTSRLYFHWRRKYQEGLC

SEQ ID NO: 169 - protein, 73 aa, Mesocricetus auratus
NKKDKHAEILFIDKMRSLELCQVRITCYLTWSPCPNCAQELAAFKKDHPDLVLRIYTSRLYFHWRRKYQEGLC

SEQ ID NO: 170 - protein, 73 aa, Microtus ochrogaster
NKKGKHAEILFIDEMRSLKLSQERITCYLTWSPCPNCAQELAAFKRDHPGLVLRIYASRLYFHWRRKYQEGLC

SEQ ID NO: 171 - protein, 72 aa, Spalax galili
  source 1..72: note = Nannospalax galili
NKRAKHAEILLIDMMRSMELGQVQITCYITWSPCPTCAQELAAFKQDHPDLVLRIYASRLYFHWKRKFQKGL

SEQ ID NO: 172 - protein, 73 aa, Meriones unguiculatus
NKKGRHAEICLIDEMRSLGLGKAQITCYLTWSPCRKCAQELATFKKDHPDLVLRVYASRLYFHWSRKYQQGLC

SEQ ID NO: 173 - protein, 75 aa, Dipodomys ordii
NKKGHHAEIRFIERIRSMGLDPSQDYQITCYLTWSPCLDCAFKLAKLKKDFPRLTLRIFTSRLYFHWIRKFQKGL

SEQ ID NO: 174 - protein, 72 aa, Jaculus jaculus
NKKGKHAEARFVDKMRSMQLDHALITCYLTWSPCLDCSQKLAALKRDHPGLTLRIFTSRLYFHWVKKFQEGL

SEQ ID NO: 175 - protein, 76 aa, Chinchilla lanigera
SPQKGHHAESRFIKRISSMDLDRSRSYQITCFLTWSPCPSCAQELASFKRAHPHLRFQIFVSRLYFHWKRSYQAGL

SEQ ID NO: 176 - protein, 74 aa, Heterocephalus glaber
KKGYHAESRFIKRICSMDLGQDQSYQVTCFLTWSPCPHCAQELVSFKRAHPHLRLQIFTARLFFHWKRSYQEGL

SEQ ID NO: 177 - protein, 74 aa, Octodon degus
KKGQHAEIRFIERIHSMALDQARSYQITCFLTWSPCPFCAQELASFKSTHPRVHLQIFVSRLYFHWKRSYQEGL

SEQ ID NO: 178 - protein, 75 aa, Urocitellus parryii
NKKGHHAEIRFIKKIRSLDLDQSQNYEVTCYLTWSPCPDCAQELVALTRSHPHVRLRLFTSRLYFHWFWSFQEGL

SEQ ID NO: 179 - protein, 72 aa, Aotus nancymai
  source 1..72: note = Aotus nancymaae
NRHAEICFIDEIESMGLDKTQCYEVTCYLTWSPCPSCAQKLAAFTKAQVHLNLRIFASRLYYHWRSSYQKGL

SEQ ID NO: 180 - protein, 72 aa, Cebus capucinus
  source 1..72: note = Cebus capucinus imitator
NRHAEICFIDEIESMGLDKTQCYEVTCYLTWSPCPSCAQKLVAFAKAQDHLNLRIFASRLYYHWRRRYKEGL

SEQ ID NO: 181 - protein, 70 aa, Saimiri boliviensis
  source 1..70: note = Saimiri boliviensis boliviensis
HVEICFIDKIASMELDKTQCYDVTCYLTWSPCPSCAQKLAAFAKAQDHLNLRIFASRLYYHWRRSYQKGL

SEQ ID NO: 182 - protein, 75 aa, Homo sapiens
NKKKCHAEICFINEIKSMGLDETQCYQVTCYLTWSPCSSCAWELVDFIKAHDHLNLGIFASRLYYHWCKPQQKGL

SEQ ID NO: 183 - protein, 76 aa, Homo sapiens
ENKKKCHAEICFINEIKSMGLDETQCYQVTCYLTWSPCSSCAWELVDFIKAHDHLNLGIFASRLYYHWCKPQQKGL

SEQ ID NO: 184 - protein, 75 aa, Pan paniscus
NKKKCHAEICFINEIKSMGLDETQCYQVTCYLTWSPCSSCAWKLVDFIQAHDHLNLRIFASRLYYHWCKPQQEGL

SEQ ID NO: 185 - protein, 75 aa, Symphalangus syndactylus
NKKKRHAEIRFINKIKSMGLDETQCYQVTCYLTWSPCPSCAWELVDFIKAHDHLNLGIFASRLYYHWCRHQQEGL

SEQ ID NO: 186 - protein, 75 aa, Macaca mulatta
NKKKDHAEIRFINKIKSMGLDETQCYQVTCYLTWSPCPSCAGELVDFIKAHRHLNLRIFASRLYYHWRPNYQEGL

SEQ ID NO: 187 - protein, 75 aa, Theropithecus gelada
NKKKEHAEIRFINKIKSMGLDETQCYQVTCYLTWSPCPSCAGKLVDFIKAHHHLNLRIFASRLYYHWRPNYQEGL

SEQ ID NO: 188 - protein, 75 aa, Mandrillus leucophaeus
NKKKHHAEIHFINKIKSMGLDETQCYQVTCYLTWSPCPSCARELVDFIKAHRHLNLRIFASRLYYHWRPHYQEGL

SEQ ID NO: 189 - protein, 75 aa, Bos grunniens
NKKQRHAEIRFIDKINSLDLNPSQSYKIICYITWSPCPNCANELVNFITRNNHLKLEIFASRLYFHWIKPFKMGL

SEQ ID NO: 190 - protein, 75 aa, Bubalus bubalis
NKKQRHAEIRFIDKINSLDLNPSQSYKIICYITWSPCPNCASELVDFITRNDHLDLQIFASRLYFHWIKPFKRGL

SEQ ID NO: 191 - protein, 75 aa, Odocoileus virginianus
  source 1..75: note = Odocoileus virginianus texanus
NKKQRHAEIRFIDKINSLNLDRRQSYKIICYITWSPCPRCASELVDFITGNDHLNLQIFASRLYFHWKKPFQRGL

SEQ ID NO: 192 - protein, 75 aa, Sus scrofa
NKKKRHAEIRFIDKINSLNLDQNQCYRIICYVTWSPCHNCAKELVDFISNRHHLSLQLFASRLYFHWVRCYQRGL

SEQ ID NO: 193 - protein, 75 aa, Ceratotherium simum
  source 1..75: note = Ceratotherium simum simum
NKKKRHAEIRFIDKIKSLGLDRVQSYEITCYITWSPCPTCALELVAFTRDYPRLSLQIFASRLYFHWRRRSIQGL

SEQ ID NO: 194 - protein, 75 aa, Equus caballus
NKKKRHAEIRFIDKINSLGLDQDQSYEITCYVTWSPCATCACKLIKFTRKFPNLSLRIFVSRLYYHWFRQNQQGL

SEQ ID NO: 195 - protein, 74 aa, Enhydra lutris
  source 1..74: note = Enhydra lutris kenyoni
KKKRHAEIRFIDSIRALQLDQSQRFEITCYLTWSPCPTCAKELAMFVQDHPHISLRLFASRLYFHWRWKYQEGL

SEQ ID NO: 196 - protein, 74 aa, Leptonychotes weddellii
KKKRHAEIRFIDNIKALRLDTSQRFEITCYVTWSPCPTCAKELVAFVRDHRHISLRLFASRLYFHWLRENKKGL

SEQ ID NO: 197 - protein, 75 aa, Enhydra lutris
  source 1..75: note = Ursus arctos horribilis
NKKKRHAEIRFIDKIRSLQRDSSQTFEITCYVTWSPCFTCAEELVAFVRDHPHVRLRLFASRLYFHWLRKYQEGL

SEQ ID NO: 198 - protein, 75 aa, Panthera leo
  source 1..75: note = Panthera leo bleyenberghi
NKKKRHAEICFIDKIKSLTRDTSQRFEIICYITWSPCPFCAEELVAFVKDNPHLSLRIFASRLYVHWRWKYQQGL

SEQ ID NO: 199 - protein, 75 aa, Panthera tigris; identical to SEQ ID NO: 198
  source 1..75: note = Panthera tigris sumatrae

SEQ ID NO: 200 - protein, 75 aa, Tupaia belangeri
NKKHRHAEVRFIAKIRSMSLDLDQKHQLTCYLTWSPCPSCAQELVTFMAESRHLNLQVFVSRLYFHWQRDFQQGL

SEQ ID NO: 201 - protein, 110 aa, Gorilla gorilla
GRSYNWLCYEVKIKRGRSNLLWNTGVFRGQMYSQPEHHAEMCFLSWFCGNQLPAYKCFQITWFVSWTPCPDCVAKLAEFLAEYPNVTLTISTARLYYYWERDYRRALCRL

SEQ ID NO: 202 - protein, 110 aa, Pan paniscus
GRSYTWLCYEVKIRRGHSNLLWDTGVFRGQMYSQPEHHAEMYFLSWFCGNQLPAYKCFQITWFVSWTPCPDCVAKLAEFLAEHPNVTLTISAARLYYYWERDYRRALCRL

SEQ ID NO: 203 - protein, 110 aa, Pan troglodytes
GRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRL

SEQ ID NO: 204 - protein, 98 aa, Gorilla gorilla
RNTVWLCYEVKTKGPSRPPLDAKIFRGQVYFEPQYHAEMCFLSWFCGNQLPAYKCFQITWFVSWTPCPDCVAKLAEFLAEHPNVTLTISAARLYYYWE

SEQ ID NO: 205 - protein, 108 aa, Pan troglodytes
RNTVWLCYEVKTKGPSRPRLDTKIFRGQVYFEPQYHAEMCFLSWFCGNQLPAYKCFQITWFVSWTPCPDCVAKLAEFLAEHPNVTLTISAARLYYYWERDYRRALCRL

SEQ ID NO: 206 - protein, 108 aa, Homo sapiens
RNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRL

SEQ ID NO: 207 - protein, 108 aa, Macaca leonina
RNTVWLCYEVKTRGPSMPTWGTKIFRGQVCFEPQYHAEMCFLSRFCGNQLPAYKRFQITWFVSWTPCPDCVAKVAEFLAEHPNVTLTISAARLYYYWETDYRRALCRL

SEQ ID NO: 208 - protein, 108 aa, Macaca nemestrina; identical to SEQ ID NO: 207

SEQ ID NO: 209 - protein, 108 aa, Rhinopithecus roxellana
RNTVWLCYEVKTRGPSMPTWGAKIFRGQVYFEPQYHAEMCFLSWFCGNQLPAYKRFQITWFVSWTPCPDCVAKVAEFLAEHPNVTLTISAARLYYYWETDYRRALCRL

SEQ ID NO: 210 - protein, 101 aa, Mandrillus leucophaeus
RNTVWLCYKVKTRGPSMPTWGTKIFRGQVYFQPQYHAEMCFLSWFCGNQLPAYKRFQITWFVSWTPCPDCVVKVAEFLAEHPNVTLTISAARLYYYWETDY

SEQ ID NO: 211 - protein, 108 aa, Macaca mulatta
RNTVWLCYEVKTRGPSMPTWDTKIFRGQVYSKPEHHAEMCFLSRFCGNQLPAYKRFQITWFVSWTPCPDCVAKVAEFLAEHPNVTLTISAARLYYYWETDYRRALCRL

SEQ ID NO: 212 - protein, 108 aa, Theropithecus gelada
RNTVWLCYEVKTRGPSMPTWGTKIFRGQVYFQPQYHAEMCFLSRFCGNQLPAYKRFQITWFVSWNPCPDCVAKVIEFLAEHPNVTLTISAARLYYYWGRDWRRALRRL

SEQ ID NO: 213 - protein, 110 aa, Cercocebus atys
GRSYTWLCYEVKIRKDPSKLPWYTGVFRGQVYSKPEHHAEMCFLSRFCGNQLPAYKRFQITWFVSWNPCPDCVAKVIEFLAEHPNVTLTISAARLYYYWSRDWQRALCRL

SEQ ID NO: 214 - protein, 110 aa, Macaca fascicularis
GRSYTWLCYEVKIRKDPSKLPWDTGVFRGQVYSKPEHHAEMCFLSRFCGNQLPAYKRFQITWFVSWNPCPDCVAKVIEFLAEHPNVTLTISTARLYYYWGRDWQRALCRL

SEQ ID NO: 215 - protein, 110 aa, Macaca mulatta; identical to SEQ ID NO: 214

SEQ ID NO: 216 - protein, 110 aa, Macaca leonina
GRSYTWLCYEVKIRKDPSKLPWDTGVFRGQVYSKPEHHAEMCFLSRFCGNQLPAYKRFQITWFVSWNPCPDCVVKVIEFLAEHPNVTLTISTARLYYYWGRDWQRALCRL

SEQ ID NO: 217 - protein, 110 aa, Mandrillus leucophaeus
GRSYTWLCYEVKIRKDPSKLPWYTGVFRGQVYSKPEHHAEMCFLSRFCGNQLPAYKRFQITWFVSWNPCPDCVAKVIEFLAEHPNVTLTIFTARLYYYWGRDWQRALCRL

SEQ ID NO: 218 - protein, 110 aa, Macaca nemestrina
GRSYTWLCYEVKIRKDPSKLPWDTGVFRGQVYSKPEHHAEMCFLSRFCGNQLPAYKRFQITWFVSWNPCPDCVAKVTEFLAEHPNVTLTISTARLYYYWGRDWQRALCRL

SEQ ID NO: 219 - protein, 110 aa, Rhinopithecus bieti
GRSYTWLCYEVKIRKDPSKLPWDTGVFRGQVYSEPEHHAEMYFLSWFCGNQLPAYKRFQITWFVSWTPCPDCVAKVAEFLTEHPNVTLTISAARLYYYRGRDWRRALCRL

SEQ ID NO: 220 - protein, 110 aa, Rhinopithecus roxellana; identical to SEQ ID NO: 219

SEQ ID NO: 221 - protein, 110 aa, Chlorocebus sabaeus
GRSYTWLCYEVKIRKDPSKLPWDTGVFRGQMYSKPEHHAEMCFLSWFCGNQLPAHKRFQITWFVSWTPCPDCVAKVAEFLAEYPNVTLTISAARLYYYWETDYRRALCRL

SEQ ID NO: 222 - protein, 109 aa, Nomascus leucogenys
RSYTWLCYEVKIRKDPSKLPWDTGVFRGQMYFQPEYHAEMCFLSWFCGNQLPAYKRFQITWFVSWTPCPDCVAKVAVFLAEHPNVTLTISAARLYYYWEKDWQRALCRL

SEQ ID NO: 223 - protein, 110 aa, Cercocebus atys
GRSYTWLCYEVKIKKYPSKLLWDTGVFQGQVYFQPQYHAEMCFLSRFCGNQLPAYKRFQITWFVSWNPCPDCVAKVTEFLAEHPNVTLTISAARLYYYWEKDXRRALRRL

SEQ ID NO: 224 - protein, 110 aa, Papio anubis
GRSYTWLCYEVKIKEDPSKLLWDTGVFQGQVYFQPQYHAEMCFLSRFCGNQLPAYKRFQITWFVSWNPCPDCVAKVTEFLAEHPNVTLTISAARLYYYWGRDWRRALRRL

SEQ ID NO: 225 - protein, 122 aa, Chlorocebus aethiops
GRRYTWLCYEVKIKKDPSKLPWDTGVFPGQVRPKFQSNRRYEVYFQPQYHAEMYFLSWFCGNQLPAYKHFQITWFVSWNPCPDCVAKVTEFLAEHRNVTLTISAARLYYYWGKDWRRALCRL

SEQ ID NO: 226 - protein, 106 aa, Chlorocebus sabaeus
GRRYTWLCYEVKIKKDPSKLPWDTGVFPGQPQYHAEMYFLSWFCGNQLPAYKHFQITWFVSWNPCPDCVAKVTEFLAEHRNVTLTISAARLYYYWGKDWRRALCRL

SEQ ID NO: 227 - protein, 122 aa, Chlorocebus sabaeus
GRRYTWLCYEVKIKKDPSKLPWDTGVFPGQVRPKFQSNRRQKVYFQPQYHAEMYFLSWFCGNQLPAYKHFQITWFVSWNPCPDCVAKVTEFLAEHRNVTLTISAARLYYYWGKDWRRALCRL

SEQ ID NO: 228 - protein, 122 aa, Erythrocebus patas
GRRYTWLCYEVKIKKDPSKLPWDTGVFQGQVRPKFQSNRRYEVYFQPQYHAEMCFLSWFCGNQLPAYKHFQITWFVSWNPCPDCVAKVTEFLAEHPNVTLTISAARLYYYWGKDWRRALCRL

SEQ ID NO: 229 - protein, 131 aa, Macaca fascicularis
GRSYTWLCYEVKIRKDPSKLPWDTGVFRGQVRPKLQSNRRYELSNWECRKRVYFQPQYHAEMYFLSWFCGNQLPANKRFQITWFASWNPCPDCVAKVTEFLAEHPNVTLTISVARLYYYRGKDWRRALRRL

SEQ ID NO: 230 - protein, 110 aa, Macaca fascicularis
GRSYTWLCYEVKIRKDPSKLPWDTGVFRGQVYFQPQYHAEMYFLSWFCGNQLPANKRFQITWFASWNPCPDCVAKVTEFLAEHPNVTLTISVARLYYYRGKDWRRALRRL

SEQ ID NO: 231 - protein, 110 aa, Macaca nemestrina
GRSYTWLCYEVKIRKDPSKLPWDTGVFRDQVYFQPQYHAEMCFLSWFCGNQLPANKRFQITWFVSWNPCPDCVTKVTEFLAEHPNVTLTISVARLYYYRGKDWRRALRRL

SEQ ID NO: 232 - protein, 110 aa, Macaca leonina
GRSYTWLCYEVKIRKDPSKLPWYTGVFRGQVYFQPQYHAEMCFLSWFCGNQLPANKRFQITWFVSWNPCPDCVAKVTEFLAEHPNVTLTISVARLYYYRGKDWRRALRRL

SEQ ID NO: 233 - protein, 110 aa, Macaca mulatta
GRSYTWLCYEVKIRKDPSKLPWDTGVFRGQVYFQPQYHAEMCFLSWFCGNQLPAYKRFQITWFVSWNPCPDCVAKVTEFLAEHPNVTLTISVARLYYYRGKDWRRALCRL

SEQ ID NO: 234 - protein, 122 aa, Gorilla gorilla
GRSYTWLCYEVKIRRGSSNLLWNTGVFRGPVPPKLQSNHRQEVYFQFENHAEMCFLSWFCGNRLPANRRFQITWFVSWNPCLPCVVKVTKFLAEHPNVTLTISAARLYYYRDREWRRVLRRL

SEQ ID NO: 235 - protein, 122 aa, Pan paniscus
GRSYTWLCYEVKIKRGCSNLIWDTGVFRGPVLPKLQSNHRQEVYFQFENHAEMCFFSWFCGNRLPANRRFQITWFVSWNPCLPCVVKVTKFLAEHPNVTLTISAARLYYYQDREWRRVLRRL

SEQ ID NO: 236 - protein, 122 aa, Pan troglodytes; identical to SEQ ID NO: 235

SEQ ID NO: 237 - protein, 122 aa, Homo sapiens
GRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRL

SEQ ID NO: 238 - protein, 122 aa, Nomascus leucogenys
GRSYTWLCYEVKIRKDPSKLPWDKGVFRGQVLPKFQSNHRQEVYFQLENHAEMCFLSWFCGNQLPANRRFQITWFVSWNPCLPCVAKVTEFLAEHPNVTLTISAARLYYYRGRDWRRALRRL

SEQ ID NO: 239 - protein, 110 aa, Saimiri boliviensis
GKKYTWLCYEVKIKKDTSKLPWNTGVFRGQVNFNPEHHAEMYFLSWFRGKLLPACKRSQITWFVSWNPCLYCVAKVAEFLAEHPNVTLTVSTARLYCYWKKDWRRALRKL

SEQ ID NO: 240 - protein, 110 aa, Saimiri boliviensis; identical to SEQ ID NO: 239

SEQ ID NO: 241 - protein, 110 aa, Piliocolobus tephrosceles
GRRYTWLCYEVKIMKDHSKLPWYTGVFRGQVYFEPQNHAEMCFLSWFCGNQLPAYECCQITWFVSWTPCPDCVAKVTEFLAEHPNVTLTISAARLYYYRGRDWRRALRRL

SEQ ID NO: 242 - protein, 110 aa, Colobus angolensis
  source 1..110: note = Colobus angolensis palliatus
GRRYTWLCYEVKISKDPSKLPWDTGIFRGQVYFEPQYHAEMCFLSWYCGNQLPAYKCFQITWFVSWTPCPDCVGKVAEFLAEHPNVTLTISAARLYYYWETDYRRALCRL

SEQ ID NO: 243 - protein, 121 aa, Pongo abelii
RNYTWLCYEVKIRKDPSKLAWDTGVFRGQVLPKLQSNHRREVYFEPQYHAEMCFLSWFCGNQLSAYERFQITWFVSWTPCPDCVAMLAEFLAEHPNVTLTVSAARLYYYWERDYRGALRRL

SEQ ID NO: 244 - RNA, 19 nt, unidentified
  source 1..19: mol_type = other RNA; note = Bacteriophage MS2
acatgaggatcacccatgt

SEQ ID NO: 245 - RNA, 25 nt, unidentified
  source 1..25: mol_type = other RNA; note = Bacteriophage PP7
ggagcagacgatatggcgtcgctcc

SEQ ID NO: 246 - RNA, 15 nt, unidentified
  source 1..15: mol_type = other RNA; note = Bacteriophage boxB
gccctgaagaagggc

SEQ ID NO: 247 - RNA, 20 nt, synthetic construct
  source 1..20: mol_type = other RNA
ctcagctgaacctggctacc

SEQ ID NO: 248 - RNA, 20 nt, synthetic construct
  source 1..20: mol_type = other RNA
agggccaggtcctggtagcc

SEQ ID NO: 249 - RNA, 20 nt, synthetic construct
  source 1..20: mol_type = other RNA
cactcacctttgcagaagac

SEQ ID NO: 250 - RNA, 20 nt, synthetic construct
  source 1..20: mol_type = other RNA
tgctagcatggaaaagccaa

SEQ ID NO: 251 - RNA, 20 nt, synthetic construct
  source 1..20: mol_type = other RNA
ggcccagcctgctgtggtac

SEQ ID NO: 252 - RNA, 20 nt, synthetic construct
  source 1..20: mol_type = other RNA
gcttcggcaggctgacagcc

SEQ ID NO: 253 - RNA, 20 nt, synthetic construct
  source 1..20: mol_type = other RNA
ccaggtgactgaagtctgtg

SEQ ID NO: 254 - RNA, 20 nt, synthetic construct
  source 1..20: mol_type = other RNA
tatccaaggactgagggcca

SEQ ID NO: 255 - RNA, 20 nt, synthetic construct
  source 1..20: mol_type = other RNA
ggaacccagatttatgtaat

SEQ ID NO: 256 - RNA, 20 nt, synthetic construct
  source 1..20: mol_type = other RNA
ctcaccaattacataaatct

SEQ ID NO: 257 - RNA, 20 nt, synthetic construct
  source 1..20: mol_type = other RNA
cataaagccatggcttgcct

SEQ ID NO: 258 - RNA, 20 nt, synthetic construct
  source 1..20: mol_type = other RNA
acaccgctcccataaagcca

SEQ ID NO: 259 - RNA, 20 nt, synthetic construct
  source 1..20: mol_type = other RNA
actcacctttgcagaagaca

SEQ ID NO: 260 - RNA, 20 nt, synthetic construct
  source 1..20: mol_type = other RNA
gaaacaaatgaaacccaggt

SEQ ID NO: 261 - RNA, 20 nt, synthetic construct
  source 1..20: mol_type = other RNA
tgctgaaacaaatgaaaccc

SEQ ID NO: 262 - RNA, 20 nt, synthetic construct
  source 1..20: mol_type = other RNA
ctgctggccagtaccacagc

SEQ ID NO: 263 - RNA, 19 nt, synthetic construct
  source 1..19: mol_type = other RNA
gctggcgatgcctcggctg

SEQ ID NO: 264 - RNA, 20 nt, synthetic construct
  source 1..20: mol_type = other RNA
acacaaagctggcgatgcct

SEQ ID NO: 265 - RNA, 20 nt, synthetic construct
  source 1..20: mol_type = other RNA
tgagttcactgagttccctt

SEQ ID NO: 266 - RNA, 20 nt, synthetic construct
  source 1..20: mol_type = other RNA
ttgcctgggcttggccatga

SEQ ID NO: 267 - RNA, 20 nt, synthetic construct
  source 1..20: mol_type = other RNA
cagaagggcttgcctgggct

SEQ ID NO: 268 - RNA, 20 nt, synthetic construct
  source 1..20: mol_type = other RNA
atctccaggcaaagccactg

SEQ ID NO: 269 - RNA, 20 nt, synthetic construct
  source 1..20: mol_type = other RNA
gtgtgtgagtatgcatctcc

SEQ ID NO: 270 - RNA, 20 nt, synthetic construct
  source 1..20: mol_type = other RNA
aggcaaagccactgaggtcc

SEQ ID NO: 271 - RNA, 20 nt, synthetic construct
  source 1..20: mol_type = other RNA
atctgcacgggcacctccag

SEQ ID NO: 272 - RNA, 20 nt, synthetic construct
  source 1..20: mol_type = other RNA
ctagatgattccatctgcac

SEQ ID NO: 273 - RNA, 20 nt, synthetic construct
  source 1..20: mol_type = other RNA
gtacccaccgccatactacc

SEQ ID NO: 274 - RNA, 20 nt, synthetic construct
  source 1..20: mol_type = other RNA
gggactctacatctgcaagg

SEQ ID NO: 275 - RNA, 20 nt, synthetic construct
  source 1..20: mol_type = other RNA
cacgggactctacatctgca

SEQ ID NO: 276 - RNA, 20 nt, synthetic construct
  source 1..20: mol_type = other RNA
atagaagactgcaatgcaac

SEQ ID NO: 277 - RNA, 20 nt, synthetic construct
  source 1..20: mol_type = other RNA
aagtaaacctcctgaaatta

SEQ ID NO: 278 - RNA, 20 nt, synthetic construct
  source 1..20: mol_type = other RNA
gagacaccagcgcatgcttc

SEQ ID NO: 279 - RNA, 20 nt, synthetic construct
  source 1..20: mol_type = other RNA
cctgggcccagatcaggagg

SEQ ID NO: 280 - RNA, 20 nt, synthetic construct
  source 1..20: mol_type = other RNA
ctgggcccaggggctgaggc

SEQ ID NO: 281 - RNA, 20 nt, synthetic construct
  source 1..20: mol_type = other RNA
aggcaggctcccctcgcctc

SEQ ID NO: 282 - RNA, 20 nt, synthetic construct
  source 1..20: mol_type = other RNA
gccttacctgaggcgagggg

SEQ ID NO: 283 - RNA, 20 nt, synthetic construct
  source 1..20: mol_type = other RNA
caggccttacctgaggcgag

SEQ ID NO: 284 - RNA, 20 nt, synthetic construct
  source 1..20: mol_type = other RNA
attcctagggaagaggacga

SEQ ID NO: 285 - RNA, 20 nt, synthetic construct
  source 1..20: mol_type = other RNA
cacaagtgacccaggtcaac

SEQ ID NO: 286 - RNA, 20 nt, synthetic construct
  source 1..20: mol_type = other RNA
agtgacccaggtcaactggg

SEQ ID NO: 287 - RNA, 20 nt, synthetic construct
  source 1..20: mol_type = other RNA
gacccaggtcaactgggagc

SEQ ID NO: 288 - RNA, 20 nt, synthetic construct
  source 1..20: mol_type = other RNA
ggagcagcaggaccagcttc

SEQ ID NO: 289 - RNA, 20 nt, synthetic construct
  source 1..20: mol_type = other RNA
aggaccagcttctggccatt

SEQ ID NO: 290 - RNA, 20 nt, synthetic construct
  source 1..20: mol_type = other RNA
gagatgtgccaccccaagtc

SEQ ID NO: 291 - RNA, 20 nt, synthetic construct
  source 1..20: mol_type = other RNA
gatcgagtggccccaggtcc

SEQ ID NO: 292 - RNA, 20 nt, synthetic construct
  source 1..20: mol_type = other RNA
caccctccagtcgctgaccg

SEQ ID NO: 293 - RNA, 20 nt, synthetic construct
  source 1..20: mol_type = other RNA
ctccagtcgctgaccgtgaa

SEQ ID NO: 294 - RNA, 20 nt, synthetic construct
  source 1..20: mol_type = other RNA
caggaatacctgagctttct

SEQ ID NO: 295 - RNA, 20 nt, synthetic construct
  source 1..20: mol_type = other RNA
caggccttacctgaggcgag

SEQ ID NO: 296 - RNA, 20 nt, synthetic construct
  source 1..20: mol_type = other RNA
tgggtttcaggccttacctg

SEQ ID NO: 297 - RNA, 20 nt, synthetic construct
  source 1..20: mol_type = other RNA
ctgctgctctgctgggtttc

SEQ ID NO: 298 - RNA, 20 nt, synthetic construct
  source 1..20: mol_type = other RNA
ctctgggcagaagcatgcgc

SEQ ID NO: 299 - RNA, 20 nt, synthetic construct
  source 1..20: mol_type = other RNA
ctgcttcctgtaggccctct

SEQ ID NO: 300 - RNA, 20 nt, synthetic construct
  source 1..20: mol_type = other RNA
aggccacatctgcttcctgt

SEQ ID NO: 301 - RNA, 20 nt, synthetic construct
  source 1..20: mol_type = other RNA
gccttgtttttcctccctgc

SEQ ID NO: 302 - RNA, 20 nt, synthetic construct
  source 1..20: mol_type = other RNA
tctccaaccaagcttagcct

SEQ ID NO: 303 - RNA, 20 nt, synthetic construct
  source 1..20: mol_type = other RNA
agcacccaagtctccaacca

SEQ ID NO: 304 - RNA, 20 nt, synthetic construct
  source 1..20: mol_type = other RNA
ttctctgcagaaatgttccc

SEQ ID NO: 305 - RNA, 20 nt, synthetic construct
  source 1..20: mol_type = other RNA
atagagccacctttctctgc

SEQ ID NO: 306 - RNA, 20 nt, synthetic construct
  source 1..20: mol_type = other RNA
gtggctctatcatcttacaa

SEQ ID NO: 307 - RNA, 20 nt, synthetic construct
  source 1..20: mol_type = other RNA
aacatttctgcagagaaagg

SEQ ID NO: 308 - RNA, 20 nt, synthetic construct
  source 1..20: mol_type = other RNA
gggaacatttctgcagagaa

SEQ ID NO: 309 - RNA, 20 nt, synthetic construct
  source 1..20: mol_type = other RNA
gtggctctatcatcttacaa

SEQ ID NO: 310 - RNA, 20 nt, synthetic construct
  source 1..20: mol_type = other RNA
atgtcacctctcctccacca

SEQ ID NO: 311 - RNA, 20 nt, synthetic construct
  source 1..20: mol_type = other RNA
caccacggcacaagtgaccc

SEQ ID NO: 312 - RNA, 20 nt, synthetic construct
  source 1..20: mol_type = other RNA
gaggcccaggccgggacctg

SEQ ID NO: 313 - RNA, 20 nt, synthetic construct
  source 1..20: mol_type = other RNA
tggagggtgaggcccaggcc

SEQ ID NO: 314 - RNA, 20 nt, synthetic construct
  source 1..20: mol_type = other RNA
gcgactggagggtgaggccc

SEQ ID NO: 315 - RNA, 20 nt, synthetic construct
  source 1..20: mol_type = other RNA
tttgtaatgctgacttgggg

SEQ ID NO: 316 - RNA, 20 nt, synthetic construct
  source 1..20: mol_type = other RNA
ccatttgtaatgctgacttg

SEQ ID NO: 317 - RNA, 20 nt, synthetic construct
  source 1..20: mol_type = other RNA
ggccatttgtaatgctgact

SEQ ID NO: 318 - RNA, 20 nt, synthetic construct
  source 1..20: mol_type = other RNA
ttcaaggatcgagtggcccc

SEQ ID NO: 319 - RNA, 20 nt, synthetic construct
  source 1..20: mol_type = other RNA
cccatccttcaaggatcgag

SEQ ID NO: 320 - RNA, 20 nt, synthetic construct
  source 1..20: mol_type = other RNA
gcacatctccccatccttca

SEQ ID NO: 321 - RNA, 20 nt, synthetic construct
  source 1..20: mol_type = other RNA
attttctatgctagagggag

SEQ ID NO: 322 - RNA, 20 nt, synthetic construct
  source 1..20: mol_type = other RNA
attgcattttctatgctaga

SEQ ID NO: 323 - RNA, 20 nt, synthetic construct
  source 1..20: mol_type = other RNA
ggaccctgcatagagagaga

SEQ ID NO: 324 - RNA, 20 nt, synthetic construct
  source 1..20: mol_type = other RNA
ggtcggtcagaatgcctatc

SEQ ID NO: 325 - RNA, 20 nt, synthetic construct
  source 1..20: mol_type = other RNA
gtcagaatgcctatctgccc

SEQ ID NO: 326 - RNA, 20 nt, synthetic construct
  source 1..20: mol_type = other RNA
tgccccagcagacgggcacg

SEQ ID NO: 327 - RNA, 20 nt, synthetic construct
  source 1..20: mol_type = other RNA
ccatttagccagtatctgga

SEQ ID NO: 328 - RNA, 20 nt, synthetic construct
  source 1..20: mol_type = other RNA
gatccaaatcccaggcataa

SEQ ID NO: 329 - RNA, 20 nt, synthetic construct
  source 1..20: mol_type = other RNA
actcggcagagagacttcac

SEQ ID NO: 330 - RNA, 20 nt, synthetic construct
  source 1..20: mol_type = other RNA
cggcagagagacttcactgc

SEQ ID NO: 331 - RNA, 20 nt, synthetic construct
  source 1..20: mol_type = other RNA
gttacctgggccatgtcccc

SEQ ID NO: 332 - RNA, 20 nt, synthetic construct
  source 1..20: mol_type = other RNA
tgctataaaaagagagagag

SEQ ID NO: 333 - RNA, 20 nt, synthetic construct
  source 1..20: mol_type = other RNA
tctgctataaaaagagagag

SEQ ID NO: 334 - RNA, 20 nt, synthetic construct
  source 1..20: mol_type = other RNA
tctctgctataaaaagagag

SEQ ID NO: 335 - RNA, 20 nt, synthetic construct
  source 1..20: mol_type = other RNA
cagagacacagacactgggg

SEQ ID NO: 336 - RNA, 20 nt, synthetic construct
  source 1..20: mol_type = other RNA
ttactgttagatttatatca

SEQ ID NO: 337 - RNA, 20 nt, synthetic construct
  source 1..20: mol_type = other RNA
cttactgttagatttatatc

SEQ ID NO: 338 - RNA, 20 nt, synthetic construct
  source 1..20: mol_type = other RNA
ggtgtagaagcagggcagat

SEQ ID NO: 339 - RNA, 20 nt, synthetic construct
  source 1..20: mol_type = other RNA
gcggctggggtgtagaagca

SEQ ID NO: 340 - RNA, 20 nt, synthetic construct
  source 1..20: mol_type = other RNA
ctcactcaccgcttgagtct

SEQ ID NO: 341 - RNA, 20 nt, synthetic construct
  source 1..20: mol_type = other RNA
tctccttctctctctatgca

SEQ ID NO: 342 - RNA, 20 nt, synthetic construct
  source 1..20: mol_type = other RNA
ctatgcagggtcctcagaag

SEQ ID NO: 343 - RNA, 20 nt, synthetic construct
  source 1..20: mol_type = other RNA
acgttgccacattcaaacac

SEQ ID NO: 344 - RNA, 20 nt, synthetic construct
  source 1..20: mol_type = other RNA
atcagtcctgagcaccacgt

SEQ ID NO: 345 - RNA, 20 nt, synthetic construct
  source 1..20: mol_type = other RNA
cacatccctttcatcagtcc

SEQ ID NO: 346 - RNA, 20 nt, synthetic construct
  source 1..20: mol_type = other RNA
agtcacattctctatggtca

SEQ ID NO: 347 - RNA, 20 nt, synthetic construct
  source 1..20: mol_type = other RNA
tgctagagtcacattctcta

SEQ ID NO: 348 - RNA, 20 nt, synthetic construct
  source 1..20: mol_type = other RNA
gtccctgaccatagagaatg

SEQ ID NO: 349 - RNA, 20 nt, synthetic construct
  source 1..20: mol_type = other RNA
accatagagaatgtgactct

SEQ ID NO: 350 - RNA, 20 nt, synthetic construct
  source 1..20: mol_type = other RNA
aatgtgactctagcagacag

SEQ ID NO: 351 - RNA, 20 nt, synthetic construct
  source 1..20: mol_type = other RNA
tcagataattttcatcctta

SEQ ID NO: 352 - RNA, 20 nt, synthetic construct
  source 1..20: mol_type = other RNA
tttcatccttatgttgtttc

SEQ ID NO: 353 - RNA, 20 nt, synthetic construct
  source 1..20: mol_type = other RNA
cttatgttgtttctgacatt

SEQ ID NO: 354 - RNA, 20 nt, synthetic construct
  source 1..20: mol_type = other RNA
gttgtttctgacattagcca

SEQ ID NO: 355 - RNA, 20 nt, synthetic construct
  source 1..20: mol_type = other RNA
attcaaaatcctctgaattc

SEQ ID NO: 356 - RNA, 20 nt, synthetic construct
  source 1..20: mol_type = other RNA
ttcccacattcaaaatcctc

SEQ ID NO: 357 - RNA, 20 nt, synthetic construct
  source 1..20: mol_type = other RNA
ctgttagatttatatcaggg

SEQ ID NO: 358 - RNA, 20 nt, synthetic construct
  source 1..20: mol_type = other RNA
ttactgttagatttatatca

SEQ ID NO: 359 - RNA, 20 nt, synthetic construct
  source 1..20: mol_type = other RNA
aaaaaaagttacttactgtt

SEQ ID NO: 360 - RNA, 20 nt, synthetic construct
  source 1..20: mol_type = other RNA
aacgttcttaactatactat

SEQ ID NO: 361 - RNA, 20 nt, synthetic construct
  source 1..20: mol_type = other RNA
atgatttcccctccaagttg

SEQ ID NO: 362 - RNA, 20 nt, synthetic construct
  source 1..20: mol_type = other RNA
aagccatgatttcccctcca

SEQ ID NO: 363 - RNA, 20 nt, synthetic construct
  source 1..20: mol_type = other RNA
atgaaggaagtctaaagcca

SEQ ID NO: 364 - RNA, 20 nt, synthetic construct
  source 1..20: mol_type = other RNA
agcctcccacatctctccta

SEQ ID NO: 365 - RNA, 20 nt, synthetic construct
  source 1..20: mol_type = other RNA
gtttctgcagccgctttggg

SEQ ID NO: 366 - RNA, 20 nt, synthetic construct
  source 1..20: mol_type = other RNA
ttacctggagccacccaaag

SEQ ID NO: 367 - RNA, 20 nt, synthetic construct
  source 1..20: mol_type = other RNA
tcactaggtgagcaaaagag

SEQ ID NO: 368 - RNA, 20 nt, synthetic construct
  source 1..20: mol_type = other RNA
gcctctccagccaggggctg

SEQ ID NO: 369 - RNA, 20 nt, synthetic construct
  source 1..20: mol_type = other RNA
tgcgaagagcaggggtcact

SEQ ID NO: 370 - RNA, 20 nt, synthetic construct
  source 1..20: mol_type = other RNA
cttggcagcatcagccagac

SEQ ID NO: 371 - RNA, 20 nt, synthetic construct
  source 1..20: mol_type = other RNA
ccactgggcgggaaagagaa

SEQ ID NO: 372 - RNA, 20 nt, synthetic construct
  source 1..20: mol_type = other RNA
ccccaggaggagggcgccgc

SEQ ID NO: 373 - RNA, 20 nt, synthetic construct
  source 1..20: mol_type = other RNA
cctgcagccccgcgtccagc

SEQ ID NO: 374 - RNA, 20 nt, synthetic construct
  source 1..20: mol_type = other RNA
cgcgtccagctggatgagcg

SEQ ID NO: 375 - RNA, 20 nt, synthetic construct
  source 1..20: mol_type = other RNA
tgggccaggcctcgagtatg

SEQ ID NO: 376 - RNA, 20 nt, synthetic construct
  source 1..20: mol_type = other RNA
acatactcgaggcctggccc

SEQ ID NO: 377 - RNA, 20 nt, synthetic construct
  source 1..20: mol_type = other RNA
cggaaccaatgcacagaggc

SEQ ID NO: 378 - RNA, 20 nt, synthetic construct
  source 1..20: mol_type = other RNA
ccagggccgagtccctgtcc

SEQ ID NO: 379 - RNA, 20 nt, synthetic construct
  source 1..20: mol_type = other RNA
cctgccccaagtcagcccca

SEQ ID NO: 380 - RNA, 20 nt, synthetic construct
  source 1..20: mol_type = other RNA
gccccagggcccagagtcca

SEQ ID NO: 381 - RNA, 20 nt, synthetic construct
  source 1..20: mol_type = other RNA
gggagttacccagaacagtg

SEQ ID NO: 382 - RNA, 20 nt, synthetic construct
  source 1..20: mol_type = other RNA
gaggagtccacttggcagtg

SEQ ID NO: 383 - RNA, 20 nt, synthetic construct
  source 1..20: mol_type = other RNA
atgtgagccaggcccaggct

SEQ ID NO: 384 - RNA, 20 nt, synthetic construct
  source 1..20: mol_type = other RNA
gagtcactgaaaagagtaga

SEQ ID NO: 385 - RNA, 20 nt, synthetic construct
  source 1..20: mol_type = other RNA
ctggacaagaacgctttgtg

SEQ ID NO: 386 - RNA, 20 nt, synthetic construct
  source 1..20: mol_type = other RNA
ccatcccagaggagtttctc

SEQ ID NO: 387 - RNA, 20 nt, synthetic construct
  source 1..20: mol_type = other RNA
cccagctcctttcccagcct

SEQ ID NO: 388 - RNA, 20 nt, synthetic construct
  source 1..20: mol_type = other RNA
ttggcaatgccagctgtacc

SEQ ID NO: 389 - RNA, 20 nt, synthetic construct
  source 1..20: mol_type = other RNA
gccagctgtaccagggggag

SEQ ID NO: 390 - RNA, 20 nt, synthetic construct
  source 1..20: mol_type = other RNA
taccagggggagaggcttct

SEQ ID NO: 391 - RNA, 20 nt, synthetic construct
  source 1..20: mol_type = other RNA
ggcacctatggagaaagtac

SEQ ID NO: 392 - RNA, 20 nt, synthetic construct
  source 1..20: mol_type = other RNA
agacaggtgagccagggaca

SEQ ID NO: 393 - RNA, 20 nt, synthetic construct
  source 1..20: mol_type = other RNA
ggctcacctgtcttctccaa

SEQ ID NO: 394 - RNA, 20 nt, synthetic construct
  source 1..20: mol_type = other RNA
gtcgccactgtgagaagaga

SEQ ID NO: 395 - RNA, 20 nt, synthetic construct
  source 1..20: mol_type = other RNA
gcaggctcagagcaagatag

SEQ ID NO: 396 - RNA, 20 nt, synthetic construct
  source 1..20: mol_type = other RNA
gctggagcaagaaccggagc

SEQ ID NO: 397 - RNA, 20 nt, synthetic construct
  source 1..20: mol_type = other RNA
ttacctggagccacccaaag

SEQ ID NO: 398 - RNA, 20 nt, synthetic construct
  source 1..20: mol_type = other RNA
cccgccatccccgttttacc

SEQ ID NO: 399 - RNA, 20 nt, synthetic construct
  source 1..20: mol_type = other RNA
gaccataggagagatgtggg

SEQ ID NO: 400 - RNA, 20 nt, synthetic construct
  source 1..20: mol_type = other RNA
cccagaccataggagagatg

SEQ ID NO: 401 - RNA, 20 nt, synthetic construct
  source 1..20: mol_type = other RNA
ctcagcgctgcccagaccat

SEQ ID NO: 402 - RNA, 20 nt, synthetic construct
  source 1..20: mol_type = other RNA
gtcccctcctgtggggctgg

SEQ ID NO: 403 - RNA, 20 nt, synthetic construct
  source 1..20: mol_type = other RNA
ccctgctggtcccctcctgt

SEQ ID NO: 404 - RNA, 20 nt, synthetic construct
  source 1..20: mol_type = other RNA
gtggccacagagatccctgc

SEQ ID NO: 405 - RNA, 20 nt, synthetic construct
  source 1..20: mol_type = other RNA
cctcctgggcccacaccacc

SEQ ID NO: 406 - RNA, 20 nt, synthetic construct
  source 1..20: mol_type = other RNA
ctgggcaggagccccctcct

SEQ ID NO: 407 - RNA, 20 nt, synthetic construct
  source 1..20: mol_type = other RNA
gggctgcaggggagctgggc

SEQ ID NO: 408 - RNA, 20 nt, synthetic construct
  source 1..20: mol_type = other RNA
cggctcacgccccctcccct

SEQ ID NO: 409 - RNA, 20 nt, synthetic construct
  source 1..20: mol_type = other RNA
ccttctctacccctgcctct

SEQ ID NO: 410 - RNA, 20 nt, synthetic construct
  source 1..20: mol_type = other RNA
ggtggtgtgggcccaggagg

SEQ ID NO: 411 - RNA, 20 nt, synthetic construct
  source 1..20: mol_type = other RNA
cagccccacaatccccctcc

SEQ ID NO: 412 - RNA, 20 nt, synthetic construct
  source 1..20: mol_type = other RNA
gccgggggccaggggatggc

SEQ ID NO: 413 - RNA, 20 nt, synthetic construct
  source 1..20: mol_type = other RNA
cgggtgagggccgggggcca

SEQ ID NO: 414 - RNA, 20 nt, synthetic construct
  source 1..20: mol_type = other RNA
gaggagggcgccgccgggtg

SEQ ID NO: 415 - RNA, 20 nt, synthetic construct
  source 1..20: mol_type = other RNA
ctcccgctgcgcaggcctcc

SEQ ID NO: 416 - RNA, 20 nt, synthetic construct
  source 1..20: mol_type = other RNA
ggggcagcctcccgctgcgc

SEQ ID NO: 417 - RNA, 20 nt, synthetic construct
  source 1..20: mol_type = other RNA
gtgctgagcgtgggtcccgg

SEQ ID NO: 418 - RNA, 20 nt, synthetic construct
  source 1..20: mol_type = other RNA
cgctacacggtgctgagcgt

SEQ ID NO: 419 - RNA, 20 nt, synthetic construct
  source 1..20: mol_type = other RNA
gcccaggccccgccgctaca

SEQ ID NO: 420 - RNA, 20 nt, synthetic construct
  source 1..20: mol_type = other RNA
ccggaggcctgcgcagcggg

SEQ ID NO: 421 - RNA, 20 nt, synthetic construct
  source 1..20: mol_type = other RNA
ccgcgccgcggtgcacctca

SEQ ID NO: 422 - RNA, 20 nt, synthetic construct
  source 1..20: mol_type = other RNA
cgccggcgagtaccgcgccg

SEQ ID NO: 423 - RNA, 20 nt, synthetic construct
  source 1..20: mol_type = other RNA
cgtcccgggaggcaggggat

SEQ ID NO: 424 - RNA, 20 nt, synthetic construct
  source 1..20: mol_type = other RNA
cttcctgcgtcccgggaggc

SEQ ID NO: 425 - RNA, 20 nt, synthetic construct
  source 1..20: mol_type = other RNA
cccagcccttcctgcgtccc

SEQ ID NO: 426 - RNA, 20 nt, synthetic construct
  source 1..20: mol_type = other RNA
gggactcccggacagggact

SEQ ID NO: 427 - RNA, 20 nt, synthetic construct
  source 1..20: mol_type = other RNA
agtggtgatggggggactcc

SEQ ID NO: 428 - RNA, 20 nt, synthetic construct
  source 1..20: mol_type = other RNA
agaggaagctttccgctaag

SEQ ID NO: 429 - RNA, 20 nt, synthetic construct
  source 1..20: mol_type = other RNA
accgcccagcctctgtgcat

SEQ ID NO: 430 - RNA, 20 nt, synthetic construct
  source 1..20: mol_type = other RNA
gtccccccatcaccacttag

SEQ ID NO: 431 - RNA, 20 nt, synthetic construct
  source 1..20: mol_type = other RNA
ccagggccgagtccctgtcc

SEQ ID NO: 432 - RNA, 20 nt, synthetic construct
  source 1..20: mol_type = other RNA
aacagtgaggttatacatga

SEQ ID NO: 433 - RNA, 20 nt, synthetic construct
  source 1..20: mol_type = other RNA
gggagttacccagaacagtg

SEQ ID NO: 434 - RNA, 20 nt, synthetic construct
  source 1..20: mol_type = other RNA
aggggggcaggaaggagttg

SEQ ID NO: 435 - RNA, 20 nt, synthetic construct
  source 1..20: mol_type = other RNA
aggtgacaaggggggcagga

SEQ ID NO: 436 - RNA, 20 nt, synthetic construct
  source 1..20: mol_type = other RNA
atagttaggggaggtgacaa

SEQ ID NO: 437 - RNA, 20 nt, synthetic construct
  source 1..20: mol_type = other RNA
cattgtctccagtcaccagg

SEQ ID NO: 438 - RNA, 20 nt, synthetic construct
  source 1..20: mol_type = other RNA
cgccattgtctccagtcacc

SEQ ID NO: 439 - RNA, 20 nt, synthetic construct
  source 1..20: mol_type = other RNA
gctcacatcctctagtcgaa

SEQ ID NO: 440 - RNA, 20 nt, synthetic construct
  source 1..20: mol_type = other RNA
ctcctggtgactggagacaa

SEQ ID NO: 441 - RNA, 20 nt, synthetic construct
  source 1..20: mol_type = other RNA
ggccctgacctcctggtgac

SEQ ID NO: 442 - RNA, 20 nt, synthetic construct
  source 1..20: mol_type = other RNA
tgggggaggccctgacctcc

SEQ ID NO: 443 - RNA, 20 nt, synthetic construct
  source 1..20: mol_type = other RNA
agcagcttccccagggatcc

SEQ ID NO: 444 - RNA, 20 nt, synthetic construct
  source 1..20: mol_type = other RNA
ctcacaaagcagcttcccca

SEQ ID NO: 445 - RNA, 20 nt, synthetic construct
  source 1..20: mol_type = other RNA
aagcgttcttgtccagatac

SEQ ID NO: 446 - RNA, 20 nt, synthetic construct
  source 1..20: mol_type = other RNA
tttgggtcacctggatccct

SEQ ID NO: 447 - RNA, 20 nt, synthetic construct
  source 1..20: mol_type = other RNA
cccaaatcctttgggtcacc

SEQ ID NO: 448 - RNA, 20 nt, synthetic construct
  source 1..20: mol_type = other RNA
ctggacaagaacgctttgtg

SEQ ID NO: 449 - RNA, 20 nt, synthetic construct
  source 1..20: mol_type = other RNA
tgtgaggtgactccagtatc

SEQ ID NO: 450 - RNA, 20 nt, synthetic construct
  source 1..20: mol_type = other RNA
cctggggaagctgctttgtg

SEQ ID NO: 451 - RNA, 20 nt, synthetic construct
  source 1..20: mol_type = other RNA
ccatcccagaggagtttctc

SEQ ID NO: 452 - RNA, 20 nt, synthetic construct
  source 1..20: mol_type = other RNA
ctctggacaccccatcccag

SEQ ID NO: 453 - RNA, 20 nt, synthetic construct
  source 1..20: mol_type = other RNA
acgctttgtgtggagctctc

SEQ ID NO: 454 - RNA, 20 nt, synthetic construct
  source 1..20: mol_type = other RNA
accttggctggaggcacagg

SEQ ID NO: 455 - RNA, 20 nt, synthetic construct
  source 1..20: mol_type = other RNA
tttctcaggaccttggctgg

SEQ ID NO: 456 - RNA, 20 nt, synthetic construct
  source 1..20: mol_type = other RNA
agaggagtttctcaggacct

SEQ ID NO: 457 - RNA, 20 nt, synthetic construct
  source 1..20: mol_type = other RNA
ggcctgctgggagggcacct

SEQ ID NO: 458 - RNA, 20 nt, synthetic construct
  source 1..20: mol_type = other RNA
caggaggtggcctgctggga

SEQ ID NO: 459 - RNA, 20 nt, synthetic construct
  source 1..20: mol_type = other RNA
ggatgagaaacagcaggagg

SEQ ID NO: 460 - RNA, 20 nt, synthetic construct
  source 1..20: mol_type = other RNA
tctctgctccttttggtgac

SEQ ID NO: 461 - RNA, 20 nt, synthetic construct
  source 1..20: mol_type = other RNA
tgtcctttctctgctccttt

SEQ ID NO: 462 - RNA, 20 nt, synthetic construct
  source 1..20: mol_type = other RNA
ctcctgctgtttctcatcct

SEQ ID NO: 463 - RNA, 20 nt, synthetic construct
  source 1..20: mol_type = other RNA
agcgggagctgctggggggc

SEQ ID NO: 464 - RNA, 20 nt, synthetic construct
  source 1..20: mol_type = other RNA
atggaagagcgggagctgct

SEQ ID NO: 465 - RNA, 20 nt, synthetic construct
  source 1..20: mol_type = other RNA
cactctgaggatggaagagc

SEQ ID NO: 466 - RNA, 20 nt, synthetic construct
  source 1..20: mol_type = other RNA
agggtgaatcccttgctcta

SEQ ID NO: 467 - RNA, 20 nt, synthetic construct
  source 1..20: mol_type = other RNA
tcttgctctgagcctgcgga

SEQ ID NO: 468 - RNA, 20 nt, synthetic construct
  source 1..20: mol_type = other RNA
tctatcttgctctgagcctg

SEQ ID NO: 469 - RNA, 20 nt, synthetic construct
  source 1..20: mol_type = other RNA
gattttctgccttagagcaa

SEQ ID NO: 470 - RNA, 20 nt, synthetic construct
  source 1..20: mol_type = other RNA
ctctccatctcttctcacag

SEQ ID NO: 471 - RNA, 20 nt, synthetic construct
  source 1..20: mol_type = other RNA
gcaagggattcaccctccgc

SEQ ID NO: 472 - RNA, 20 nt, synthetic construct
  source 1..20: mol_type = other RNA
ggacgaggtctagaaggcag

SEQ ID NO: 473 - RNA, 20 nt, synthetic construct
  source 1..20: mol_type = other RNA
gtcatgcagcccttgcctgc

SEQ ID NO: 474 - RNA, 20 nt, synthetic construct
  source 1..20: mol_type = other RNA
catgcagcccttgcctgctg

SEQ ID NO: 475 - RNA, 20 nt, synthetic construct
  source 1..20: mol_type = other RNA
ccagacagagagtgagccaa

SEQ ID NO: 476 - RNA, 20 nt, synthetic construct
  source 1..20: mol_type = other RNA
cagactcaccagattcccga

SEQ ID NO: 477 - RNA, 20 nt, synthetic construct
  source 1..20: mol_type = other RNA
ctcaccagattcccgaaggt

SEQ ID NO: 478 - RNA, 20 nt, synthetic construct
  source 1..20: mol_type = other RNA
accagcctaggcaagtgcag

SEQ ID NO: 479 - RNA, 20 nt, synthetic construct
  source 1..20: mol_type = other RNA
agcctaggcaagtgcagagg

SEQ ID NO: 480 - RNA, 20 nt, synthetic construct
  source 1..20: mol_type = other RNA
tggaaccccaataccagcct

SEQ ID NO: 481 - RNA, 20 nt, synthetic construct
  source 1..20: mol_type = other RNA
cacctgcagaagatgccaga

SEQ ID NO: 482 - RNA, 20 nt, synthetic construct
  source 1..20: mol_type = other RNA
cagccttgtgcagcactatg

SEQ ID NO: 483 - RNA, 20 nt, synthetic construct
  source 1..20: mol_type = other RNA
tccagggacggggcccacag

SEQ ID NO: 484 - RNA, 20 nt, synthetic construct
  source 1..20: mol_type = other RNA
cttgggcagttccagggacg

SEQ ID NO: 485 - RNA, 20 nt, synthetic construct
  source 1..20: mol_type = other RNA
gactggcttgggcagttcca

SEQ ID NO: 486 - RNA, 20 nt, synthetic construct
  source 1..20: mol_type = other RNA
gcccctgtgggccccgtccc

SEQ ID NO: 487 - RNA, 20 nt, synthetic construct
  source 1..20: mol_type = other RNA
gactgggcagcggcccctgt

SEQ ID NO: 488 - RNA, 20 nt, synthetic construct
  source 1..20: mol_type = other RNA
ctgtggagcggactgggcag

SEQ ID NO: 489 - RNA, 20 nt, synthetic construct
  source 1..20: mol_type = other RNA
tgctggggccttcctcgagg

SEQ ID NO: 490 - RNA, 20 nt, synthetic construct
  source 1..20: mol_type = other RNA
gcctgctggggccttcctcg

SEQ ID NO: 491 - RNA, 20 nt, synthetic construct
  source 1..20: mol_type = other RNA
catgcagcccttgcctgctg

SEQ ID NO: 492 - RNA, 20 nt, synthetic construct
  source 1..20: mol_type = other RNA
ccaccagactactcaggaaa

SEQ ID NO: 493 - RNA, 20 nt, synthetic construct
  source 1..20: mol_type = other RNA
cttctcccaccagactactc

SEQ ID NO: 494 - RNA, 20 nt, synthetic construct
  source 1..20: mol_type = other RNA
caggtgttgtcgggcctcgc

SEQ ID NO: 495 - RNA, 21 nt, synthetic construct
  source 1..21: mol_type = other RNA
catcttctgcaggtgttgtcg

SEQ ID NO: 496 - RNA, 20 nt, synthetic construct
  source 1..20: mol_type = other RNA
tgccttctggcatcttctgc

SEQ ID NO: 497 - RNA, 20 nt, synthetic construct
  source 1..20: mol_type = other RNA
cgtactaagaacgtgccttc

SEQ ID NO: 498 - RNA, 20 nt, synthetic construct
  source 1..20: mol_type = other RNA
ctggtattggggttccatta

SEQ ID NO: 499 - RNA, 20 nt, synthetic construct
  source 1..20: mol_type = other RNA
acttgcctaggctggtattg

SEQ ID NO: 500 - RNA, 20 nt, synthetic construct
  source 1..20: mol_type = other RNA
ccctctgcacttgcctaggc

SEQ ID NO: 501 - RNA, 20 nt, synthetic construct
  source 1..20: mol_type = other RNA
cttgtccaggccacgcatcc

SEQ ID NO: 502 - RNA, 20 nt, synthetic construct
  source 1..20: mol_type = other RNA
tggactccaactgcttgtcc

SEQ ID NO: 503 - RNA, 20 nt, synthetic construct
  source 1..20: mol_type = other RNA
tgccgactccagcttccgtc

SEQ ID NO: 504 - RNA, 20 nt, synthetic construct
  source 1..20: mol_type = other RNA
agcagaagctgagttcaacc

SEQ ID NO: 505 - RNA, 20 nt, synthetic construct
  source 1..20: mol_type = other RNA
ttcagagcagtttgagacag

SEQ ID NO: 506 - RNA, 20 nt, synthetic construct
  source 1..20: mol_type = other RNA
actccagttcctgacggctg

SEQ ID NO: 507 - RNA, 20 nt, synthetic construct
  source 1..20: mol_type = other RNA
acctacaggagtacctgacg

SEQ ID NO: 508 - RNA, 20 nt, synthetic construct
  source 1..20: mol_type = other RNA
ttacctgcccactgttagcc

SEQ ID NO: 509 - RNA, 20 nt, synthetic construct
  source 1..20: mol_type = other RNA
ttcccagagcaccagagcca

SEQ ID NO: 510 - RNA, 20 nt, synthetic construct
  source 1..20: mol_type = other RNA
agccagaagctgggaatttc

SEQ ID NO: 511 - RNA, 20 nt, synthetic construct
  source 1..20: mol_type = other RNA
atcagcctcctgccaccact

SEQ ID NO: 512 - RNA, 20 nt, synthetic construct
  source 1..20: mol_type = other RNA
catcagcctcctgccaccac

SEQ ID NO: 513 - RNA, 20 nt, synthetic construct
  source 1..20: mol_type = other RNA
tcgctttgctgaggtctata

SEQ ID NO: 514 - RNA, 20 nt, synthetic construct
  source 1..20: mol_type = other RNA
ggggaaaggtcgctttgctg

SEQ ID NO: 515 - RNA, 20 nt, synthetic construct
  source 1..20: mol_type = other RNA
ctggacaccctggtggggaa

SEQ ID NO: 516 - RNA, 20 nt, synthetic construct
  source 1..20: mol_type = other RNA
ttgagctggacaccctggtg

SEQ ID NO: 517 - RNA, 20 nt, synthetic construct
  source 1..20: mol_type = other RNA
tgcctcttggaagacagaga

SEQ ID NO: 518 - RNA, 20 nt, synthetic construct
  source 1..20: mol_type = other RNA
cctatgaggagtatgcctct

SEQ ID NO: 519 - RNA, 20 nt, synthetic construct
  source 1..20: mol_type = other RNA
cggagttggggaaacaatac

SEQ ID NO: 520 - RNA, 20 nt, synthetic construct
  source 1..20: mol_type = other RNA
aggagcggaagacggagttg

SEQ ID NO: 521 - RNA, 20 nt, synthetic construct
  source 1..20: mol_type = other RNA
gaggagcggaagacggagtt

SEQ ID NO: 522 - RNA, 20 nt, synthetic construct
  source 1..20: mol_type = other RNA
tgaggagcggaagacggagt

SEQ ID NO: 523 - RNA, 20 nt, synthetic construct
  source 1..20: mol_type = other RNA
aggccaggctcaaggtaaag

SEQ ID NO: 524 - RNA, 20 nt, synthetic construct
  source 1..20: mol_type = other RNA
gtagggtgaggccaggctca

SEQ ID NO: 525 - RNA, 20 nt, synthetic construct
  source 1..20: mol_type = other RNA
tcaagaggtagggtgaggcc

SEQ ID NO: 526 - RNA, 20 nt, synthetic construct
  source 1..20: mol_type = other RNA
atggatcaagaggtagggtg

SEQ ID NO: 527 - RNA, 20 nt, synthetic construct
  source 1..20: mol_type = other RNA
ttaccactacacaatgatgc

SEQ ID NO: 528 - RNA, 20 nt, synthetic construct
  source 1..20: mol_type = other RNA
ctgccacctaagaggcaact

SEQ ID NO: 529 - RNA, 20 nt, synthetic construct
  source 1..20: mol_type = other RNA
agaattctctgccacctaag

SEQ ID NO: 530 - RNA, 20 nt, synthetic construct
  source 1..20: mol_type = other RNA
gagggtccagatgcccagca

SEQ ID NO: 531 - RNA, 20 nt, synthetic construct
  source 1..20: mol_type = other RNA
agggctcaccagaggtagga

SEQ ID NO: 532 - RNA, 20 nt, synthetic construct
  source 1..20: mol_type = other RNA
gttgagactcagaacttgga

SEQ ID NO: 533 - RNA, 20 nt, synthetic construct
  source 1..20: mol_type = other RNA
tacctggaggacagggctta

SEQ ID NO: 534 - RNA, 20 nt, synthetic construct
  source 1..20: mol_type = other RNA
ttcacctgcccaaggaaaaa

SEQ ID NO: 535 - RNA, 20 nt, synthetic construct
  source 1..20: mol_type = other RNA
ctaagcctagaaaatcagtt

SEQ ID NO: 536 - RNA, 20 nt, synthetic construct
  source 1..20: mol_type = other RNA
acatctagaaaaaaaaatac

SEQ ID NO: 537 - RNA, 20 nt, synthetic construct
  source 1..20: mol_type = other RNA
attaccttcctctttgcact

SEQ ID NO: 538 - RNA, 20 nt, synthetic construct
  source 1..20: mol_type = other RNA
gatcctgtaggttggaacat

SEQ ID NO: 539 - RNA, 20 nt, synthetic construct
  source 1..20: mol_type = other RNA
aagccaccccaagttagatc

SEQ ID NO: 540 - RNA, 20 nt, synthetic construct
  source 1..20: mol_type = other RNA
ccccaaacaattagtggaat

SEQ ID NO: 541 - RNA, 20 nt, synthetic construct
  source 1..20: mol_type = other RNA
aacttaccccaaacaattag

SEQ ID NO: 542 - RNA, 20 nt, synthetic construct
  source 1..20: mol_type = other RNA
gcctccacccgggcaggaga

SEQ ID NO: 543 - RNA, 20 nt, synthetic construct
  source 1..20: mol_type = other RNA
gggtaagcctccacccgggc

SEQ ID NO: 544 - RNA, 20 nt, synthetic construct
  source 1..20: mol_type = other RNA
gacggggtaagcctccaccc

SEQ ID NO: 545 - RNA, 20 nt, synthetic construct
  source 1..20: mol_type = other RNA
cctatccccgggactaagac

SEQ ID NO: 546 - RNA, 20 nt, synthetic construct
  source 1..20: mol_type = other RNA
gccccactttgcctatcccc

SEQ ID NO: 547 - RNA, 20 nt, synthetic construct
  source 1..20: mol_type = other RNA
agtgactgacatcaactcca

SEQ ID NO: 548 - RNA, 20 nt, synthetic construct
  source 1..20: mol_type = other RNA
gtgactgacatcaactccaa

SEQ ID NO: 549 - RNA, 20 nt, synthetic construct
  source 1..20: mol_type = other RNA
tgacatcaactccaagggat

SEQ ID NO: 550 - RNA, 20 nt, synthetic construct
  source 1..20: mol_type = other RNA
gtgactttcactgtaatctc

SEQ ID NO: 551 - RNA, 20 nt, synthetic construct
  source 1..20: mol_type = other RNA
tcttggcagggcacgcagtc

SEQ ID NO: 552 - RNA, 20 nt, synthetic construct
  source 1..20: mol_type = other RNA
tactccttcccttcttggca

SEQ ID NO: 553 - RNA, 20 nt, synthetic construct
  source 1..20: mol_type = other RNA
ctgtgtactccttcccttct

SEQ ID NO: 554 - RNA, 20 nt, synthetic construct
  source 1..20: mol_type = other RNA
gcacttggtattctgggtcc

SEQ ID NO: 555 - RNA, 20 nt, synthetic construct
  source 1..20: mol_type = other RNA
tacatctgcacttggtattc

SEQ ID NO: 556 - RNA, 20 nt, synthetic construct
  source 1..20: mol_type = other RNA
gtttggtttacatctgcact

SEQ ID NO: 557 - RNA, 20 nt, synthetic construct
  source 1..20: mol_type = other RNA
ctctttgcacttggtgttgc

SEQ ID NO: 558 - RNA, 20 nt, synthetic construct
  source 1..20: mol_type = other RNA
attaccttcctctttgcact

SEQ ID NO: 559 - RNA, 20 nt, synthetic construct
  source 1..20: mol_type = other RNA
tttccatggggttgggggaa

SEQ ID NO: 560 - RNA, 20 nt, synthetic construct
  source 1..20: mol_type = other RNA
tcacatctttccatggggtt

SEQ ID NO: 561 - RNA, 20 nt, synthetic construct
  source 1..20: mol_type = other RNA
tttttcttcacatctttcca

SEQ ID NO: 562 - RNA, 20 nt, synthetic construct
  source 1..20: mol_type = other RNA
ccccaaacaattagtggaat

SEQ ID NO: 563 - RNA, 20 nt, synthetic construct
  source 1..20: mol_type = other RNA
aacttaccccaaacaattag

SEQ ID NO: 564 - RNA, 20 nt, synthetic construct
  source 1..20: mol_type = other RNA
gctttttataggtaagaatg

SEQ ID NO: 565 - RNA, 20 nt, synthetic construct
  source 1..20: mol_type = other RNA
gcccaaggtaagagcttccc

SEQ ID NO: 566 - RNA, 20 nt, synthetic construct
  source 1..20: mol_type = other RNA
gctcttaccttgggcagcca

SEQ ID NO: 567 - RNA, 20 nt, synthetic construct
  source 1..20: mol_type = other RNA
tgcacctctggggaggacct

SEQ ID NO: 568 - RNA, 20 nt, synthetic construct
  source 1..20: mol_type = other RNA
ccaagacatcatttactacg

SEQ ID NO: 569 - RNA, 20 nt, synthetic construct
  source 1..20: mol_type = other RNA
cgcctgcagctgtcggacac

SEQ ID NO: 570 - RNA, 20 nt, synthetic construct
  source 1..20: mol_type = other RNA
cacctgccaggccatcacgg

SEQ ID NO: 571 - RNA, 20 nt, synthetic construct
  source 1..20: mol_type = other RNA
cctacctgtcaccaggacca

SEQ ID NO: 572 - RNA, 20 nt, synthetic construct
  source 1..20: mol_type = other RNA
ccaaggatggcacagatgct

SEQ ID NO: 573 - RNA, 20 nt, synthetic construct
  source 1..20: mol_type = other RNA
cagaggaacagtcccaagga

SEQ ID NO: 574 - RNA, 20 nt, synthetic construct
  source 1..20: mol_type = other RNA
cctctgagaaggaaaaaaga

SEQ ID NO: 575 - RNA, 20 nt, synthetic construct
  source 1..20: mol_type = other RNA
accagtaccagtgacccagt

SEQ ID NO: 576 - RNA, 20 nt, synthetic construct
  source 1..20: mol_type = other RNA
ctgcttctggcgctggctcg

SEQ ID NO: 577 - RNA, 20 nt, synthetic construct
  source 1..20: mol_type = other RNA
gctgcccctgcttctggcgc

SEQ ID NO: 578 - RNA, 20 nt, synthetic construct
  source 1..20: mol_type = other RNA
cctgctgctgcccctgcttc

SEQ ID NO: 579 - RNA, 20 nt, synthetic construct
  source 1..20: mol_type = other RNA
ggaacatggccgggcctccg

SEQ ID NO: 580 - RNA, 20 nt, synthetic construct
  source 1..20: mol_type = other RNA
cagggagagctctggagctg

SEQ ID NO: 581 - RNA, 20 nt, synthetic construct
  source 1..20: mol_type = other RNA
tcatggggcagggagagctc

SEQ ID NO: 582 - RNA, 20 nt, synthetic construct
  source 1..20: mol_type = other RNA
gggctctgagctcatggggc

SEQ ID NO: 583 - RNA, 20 nt, synthetic construct
  source 1..20: mol_type = other RNA
gactgggggctctgagctca

SEQ ID NO: 584 - RNA, 20 nt, synthetic construct
  source 1..20: mol_type = other RNA
tgttgacggaggctcccacg

SEQ ID NO: 585 - RNA, 20 nt, synthetic construct
  source 1..20: mol_type = other RNA
gatgttgacggaggctccca

SEQ ID NO: 586 - RNA, 20 nt, synthetic construct
  source 1..20: mol_type = other RNA
ggagcaggtgatgttgacgg

SEQ ID NO: 587 - RNA, 20 nt, synthetic construct
  source 1..20: mol_type = other RNA
ggtggagcaggtgatgttga

SEQ ID NO: 588 - RNA, 20 nt, synthetic construct
  source 1..20: mol_type = other RNA
ccaccagcgggggcctgcgt

SEQ ID NO: 589 - RNA, 20 nt, synthetic construct
  source 1..20: mol_type = other RNA
atcacctgctccaccagcgg

SEQ ID NO: 590 - RNA, 20 nt, synthetic construct
  source 1..20: mol_type = other RNA
aacatcacctgctccaccag

SEQ ID NO: 591 - RNA, 20 nt, synthetic construct
  source 1..20: mol_type = other RNA
catcgacttctcagggtccc

SEQ ID NO: 592 - RNA, 20 nt, synthetic construct
  source 1..20: mol_type = other RNA
ggggccgcatcgacttctca

SEQ ID NO: 593 - RNA, 20 nt, synthetic construct
  source 1..20: mol_type = other RNA
actacggacagacggttccg

SEQ ID NO: 594 - RNA, 20 nt, synthetic construct
  source 1..20: mol_type = other RNA
catgcaccgcctgcagctgt

SEQ ID NO: 595 - RNA, 20 nt, synthetic construct
  source 1..20: mol_type = other RNA
agcagctggggttgggaggg

SEQ ID NO: 596 - RNA, 20 nt, synthetic construct
  source 1..20: mol_type = other RNA
ggccagcagctggggttggg

SEQ ID NO: 597 - RNA, 20 nt, synthetic construct
  source 1..20: mol_type = other RNA
gcctggccagcagctggggt

SEQ ID NO: 598 - RNA, 20 nt, synthetic construct
  source 1..20: mol_type = other RNA
cagagcctggccagcagctg

SEQ ID NO: 599 - RNA, 20 nt, synthetic construct
  source 1..20: mol_type = other RNA
gctctaacaggttctccacc

SEQ ID NO: 600 - RNA, 20 nt, synthetic construct
  source 1..20: mol_type = other RNA
caaaccccgggggctctaac

SEQ ID NO: 601 - RNA, 20 nt, synthetic construct
  source 1..20: mol_type = other RNA
ccccgaatcccaaaccccgg

SEQ ID NO: 602 - RNA, 20 nt, synthetic construct
  source 1..20: mol_type = other RNA
gagggcagaggcccttggtg

SEQ ID NO: 603 - RNA, 20 nt, synthetic construct
  source 1..20: mol_type = other RNA
gcagggagggcagaggccct

SEQ ID NO: 604 - RNA, 20 nt, synthetic construct
  source 1..20: mol_type = other RNA
cggtggggcagggagggcag

SEQ ID NO: 605 - RNA, 20 nt, synthetic construct
  source 1..20: mol_type = other RNA
gcctgtcggtggggcaggga

SEQ ID NO: 606 - RNA, 20 nt, synthetic construct
  source 1..20: mol_type = other RNA
ggcggcatgtgtggtgtacg

SEQ ID NO: 607 - RNA, 20 nt, synthetic construct
  source 1..20: mol_type = other RNA
taagaattcggcggcatgtg

SEQ ID NO: 608 - RNA, 20 nt, synthetic construct
  source 1..20: mol_type = other RNA
gtggcgggataagaattcgg

SEQ ID NO: 609 - RNA, 20 nt, synthetic construct
  source 1..20: mol_type = other RNA
gccatctggggctgaaaggc

SEQ ID NO: 610 - RNA, 20 nt, synthetic construct
  source 1..20: mol_type = other RNA
gctaaccaataaggggtggc

SEQ ID NO: 611 - RNA, 20 nt, synthetic construct
  source 1..20: mol_type = other RNA
cacctttctttaaatcaacg

SEQ ID NO: 612 - RNA, 20 nt, synthetic construct
  source 1..20: mol_type = other RNA
agcaagctgccgcagatcgc

SEQ ID NO: 613 - RNA, 20 nt, synthetic construct
  source 1..20: mol_type = other RNA
gagcttccaagtcttctcca

SEQ ID NO: 614 - RNA, 20 nt, synthetic construct
  source 1..20: mol_type = other RNA
ccaccagattagctctggcc

SEQ ID NO: 615 - RNA, 20 nt, synthetic construct
  source 1..20: mol_type = other RNA
taaacttacctgaaacagcc

SEQ ID NO: 616 - RNA, 20 nt, synthetic construct
  source 1..20: mol_type = other RNA
ttatttcaagccctgattga

SEQ ID NO: 617 - RNA, 20 nt, synthetic construct
  source 1..20: mol_type = other RNA
ttacctgtgtaacttttata

SEQ ID NO: 618 - RNA, 20 nt, synthetic construct
  source 1..20: mol_type = other RNA
attatacctgccatgccgta

SEQ ID NO: 619 - RNA, 20 nt, synthetic construct
  source 1..20: mol_type = other RNA
accaataaggggtggcaggt

SEQ ID NO: 620 - RNA, 20 nt, synthetic construct
  source 1..20: mol_type = other RNA
acgaaggagctaaccaataa

SEQ ID NO: 621 - RNA, 20 nt, synthetic construct
  source 1..20: mol_type = other RNA
cgggaaaggaaatggacgaa

SEQ ID NO: 622 - RNA, 20 nt, synthetic construct
  source 1..20: mol_type = other RNA
aacgaaagtggaaacgggaa

SEQ ID NO: 623 - RNA, 20 nt, synthetic construct
  source 1..20: mol_type = other RNA
gactctcccgggaacgaaag

SEQ ID NO: 624 - RNA, 20 nt, synthetic construct
  source 1..20: mol_type = other RNA
ctgttgtcaacaaaagcgca

SEQ ID NO: 625 - RNA, 20 nt, synthetic construct
  source 1..20: mol_type = other RNA
gggtattattgatgctattc

SEQ ID NO: 626 - RNA, 20 nt, synthetic construct
  source 1..20: mol_type = other RNA
ccccgaaaaggtcgaatttt

SEQ ID NO: 627 - RNA, 20 nt, synthetic construct
  source 1..20: mol_type = other RNA
aagtgaatagtgtttcgcac

SEQ ID NO: 628 - RNA, 20 nt, synthetic construct
  source 1..20: mol_type = other RNA
tgcacagaactatcgtacca

SEQ ID NO: 629 - RNA, 20 nt, synthetic construct
  source 1..20: mol_type = other RNA
agggagagagaagaagggaa

SEQ ID NO: 630 - RNA, 20 nt, synthetic construct
  source 1..20: mol_type = other RNA
aagacagggagagagaagaa

SEQ ID NO: 631 - RNA, 20 nt, synthetic construct
  source 1..20: mol_type = other RNA
agaaacagagagaaaagaca

SEQ ID NO: 632 - RNA, 20 nt, synthetic construct
  source 1..20: mol_type = other RNA
ttggctatgtgactggggat

SEQ ID NO: 633 - RNA, 20 nt, synthetic construct
  source 1..20: mol_type = other RNA
gggccattggctatgtgact

SEQ ID NO: 634 - RNA, 20 nt, synthetic construct
  source 1..20: mol_type = other RNA
tatttatttcaagggcatta

SEQ ID NO: 635 - RNA, 20 nt, synthetic construct
  source 1..20: mol_type = other RNA
gagagtcttatttatttcaa

SEQ ID NO: 636 - RNA, 20 nt, synthetic construct
  source 1..20: mol_type = other RNA
ctgaaaagagaaatattagt

SEQ ID NO: 637 - RNA, 20 nt, synthetic construct
  source 1..20: mol_type = other RNA
aggcagcaacgaaaacctaa

SEQ ID NO: 638 - RNA, 20 nt, synthetic construct
  source 1..20: mol_type = other RNA
tgaacaggaaataacctatg

SEQ ID NO: 639 - RNA, 20 nt, synthetic construct
  source 1..20: mol_type = other RNA
aaaccttcaaaaagcttctc

SEQ ID NO: 640 - RNA, 20 nt, synthetic construct
  source 1..20: mol_type = other RNA
aagcttctcaggattttcaa

SEQ ID NO: 641 - RNA, 20 nt, synthetic construct
  source 1..20: mol_type = other RNA
taaatctgcagggagagaaa

SEQ ID NO: 642 - RNA, 20 nt, synthetic construct
  source 1..20: mol_type = other RNA
actgcagagatggataacca

SEQ ID NO: 643 - RNA, 20 nt, synthetic construct
  source 1..20: mol_type = other RNA
gacatcacacactgcagaga

SEQ ID NO: 644 - RNA, 20 nt, synthetic construct
  source 1..20: mol_type = other RNA
aggcagcaacgaaaacctaa

SEQ ID NO: 645 - RNA, 20 nt, synthetic construct
  source 1..20: mol_type = other RNA
atctgcccccaaacccaaag

SEQ ID NO: 646 - RNA, 20 nt, synthetic construct
  source 1..20: mol_type = other RNA
aggcagcaacgaaaacctaa

SEQ ID NO: 647 - RNA, 20 nt, synthetic construct
  source 1..20: mol_type = other RNA
ctccattttagcaactgaac

SEQ ID NO: 648 - RNA, 20 nt, synthetic construct
  source 1..20: mol_type = other RNA
tgaacaggaaataacctatg

SEQ ID NO: 649 - RNA, 20 nt, synthetic construct
  source 1..20: mol_type = other RNA
ctccattttagcaactgaac

SEQ ID NO: 650 - RNA, 20 nt, synthetic construct
  source 1..20: mol_type = other RNA
agataagacagataattccc

SEQ ID NO: 651 - RNA, 20 nt, synthetic construct
  source 1..20: mol_type = other RNA
cgccagcagtggagcggtcc

SEQ ID NO: 652 - RNA, 20 nt, synthetic construct
  source 1..20: mol_type = other RNA
ctccactgctggcgccacct

SEQ ID NO: 653 - RNA, 20 nt, synthetic construct
  source 1..20: mol_type = other RNA
ctatcagccactaatgaagt

SEQ ID NO: 654 - RNA, 20 nt, synthetic construct
  source 1..20: mol_type = other RNA
aattaccttgttgcaaggta

SEQ ID NO: 655 - RNA, 20 nt, synthetic construct
  source 1..20: mol_type = other RNA
ctgctccaaagaagaatcta

SEQ ID NO: 656 - RNA, 20 nt, synthetic construct
  source 1..20: mol_type = other RNA
atcagttcacacaggtccag

SEQ ID NO: 657 - RNA, 20 nt, synthetic construct
  source 1..20: mol_type = other RNA
ttttccagaatactgaaaca

SEQ ID NO: 658 - RNA, 20 nt, synthetic construct
  source 1..20: mol_type = other RNA
aggttcagacactagaggcc

SEQ ID NO: 659 - RNA, 20 nt, synthetic construct
  source 1..20: mol_type = other RNA
ttacctgtagatattcttgc

SEQ ID NO: 660 - RNA, 20 nt, synthetic construct
  source 1..20: mol_type = other RNA
gatcctcgtcgtggtgctcg

SEQ ID NO: 661 - RNA, 20 nt, synthetic construct
  source 1..20: mol_type = other RNA
cctggtcctgatcctcgtcg

SEQ ID NO: 662 - RNA, 20 nt, synthetic construct
  source 1..20: mol_type = other RNA
ctgtcttggcgtcagtatcc

SEQ ID NO: 663 - RNA, 20 nt, synthetic construct
  source 1..20: mol_type = other RNA
catcgcgccaggacggtctc

SEQ ID NO: 664 - RNA, 20 nt, synthetic construct
  source 1..20: mol_type = other RNA
cttgacgcatcgcgccagga

SEQ ID NO: 665 - RNA, 20 nt, synthetic construct
  source 1..20: mol_type = other RNA
tgtacttgacgcatcgcgcc

SEQ ID NO: 666 - RNA, 20 nt, synthetic construct
  source 1..20: mol_type = other RNA
agtgtatgggatgctttcaa

SEQ ID NO: 667 - RNA, 20 nt, synthetic construct
  source 1..20: mol_type = other RNA
gacctatgaattgttaccat

SEQ ID NO: 668 - RNA, 20 nt, synthetic construct
  source 1..20: mol_type = other RNA
gctggacctgtgtgaactga

SEQ ID NO: 669 - RNA, 20 nt, synthetic construct
  source 1..20: mol_type = other RNA
ccagggtgaacatgtcccgc

SEQ ID NO: 670 - RNA, 20 nt, synthetic construct
  source 1..20: mol_type = other RNA
gcctagcagcgtgtcctcca

SEQ ID NO: 671 - RNA, 20 nt, synthetic construct
  source 1..20: mol_type = other RNA
ttcttccttagattcttctt

SEQ ID NO: 672 - RNA, 20 nt, synthetic construct
  source 1..20: mol_type = other RNA
agctctagaatttcactact

SEQ ID NO: 673 - RNA, 20 nt, synthetic construct
  source 1..20: mol_type = other RNA
gagtaacgtcttaagttctg

SEQ ID NO: 674 - RNA, 20 nt, synthetic construct
  source 1..20: mol_type = other RNA
ttttagcacttttgggagtg

SEQ ID NO: 675 - RNA, 20 nt, synthetic construct
  source 1..20: mol_type = other RNA
ttcttctattttagcacttt

SEQ ID NO: 676 - RNA, 20 nt, synthetic construct
  source 1..20: mol_type = other RNA
aggaaaaaaggaaattctac

SEQ ID NO: 677 - RNA, 20 nt, synthetic construct
  source 1..20: mol_type = other RNA
cagaggaggctaaggaaaaa

SEQ ID NO: 678 - RNA, 20 nt, synthetic construct
  source 1..20: mol_type = other RNA
tccaagctcagaggaggcta

SEQ ID NO: 679 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
ctggatatctgtgggacaaggtttgagagctagggccctgaagaagggccctagcaagttcaaataaggctagtccgttatcaacttgggccctgaagaagggcccaagtggcaccgagtcggtgc

SEQ ID NO: 680 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
agagtctctcagctggtacagtttgagagctaggccaacatgaggatcacccatgtctgcagggcctagcaagttcaaataaggctagtccgttatcaacttgaaaaagtggcaccgagtcggtgc

SEQ ID NO: 681 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
agctggtacagtttgagagctaggccaacatgaggatcacccatgtctgcagggcctagcaagttcaaataaggctagtccgttatcaacttgaaaaagtggcaccgagtcggtgc

SEQ ID NO: 682 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
ttcgtatctgtaaaaccaaggtttgagagctagggccctgaagaagggccctagcaagttcaaataaggctagtccgttatcaacttgggccctgaagaagggcccaagtggcaccgagtcggtgc

SEQ ID NO: 683 - RNA, 111 nt, synthetic construct
  source 1..111: mol_type = other RNA
  modified_base 1..3: mod_base = OTHER; note = 2'-O-methylated nucleotide
  modified_base 109..111: mod_base = OTHER; note = 2'-O-methylated nucleotide
  modified_base 1..4: mod_base = OTHER; note = there is a 3'-phosphorothioate linkage between two adjoining nucleotides
  modified_base 108..111: mod_base = OTHER; note = there is a 3'-phosphorothioate linkage between two adjoining nucleotides
ttcgtatctgtaaaaccaaggtttgagagctagggccctgaagaagggccctagcaagttcaaataaggctagtccgttatcaacttgaaaaagtggcaccgagtcggtgc

SEQ ID NO: 684 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
ccactttcaggaggaggattgtttgagagctaggccaacatgaggatcacccatgtctgcagggcctagcaagttcaaataaggctagtccgttatcaacttgaaaaagtggcaccgagtcggtgc

SEQ ID NO: 685 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
gaggaggattgtttgagagctaggccaacatgaggatcacccatgtctgcagggcctagcaagttcaaataaggctagtccgttatcaacttgaaaaagtggcaccgagtcggtgc

SEQ ID NO: 686 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
ctcttacctgtaccataaccgtttgagagctagggccctgaagaagggccctagcaagttcaaataaggctagtccgttatcaacttgggccctgaagaagggcccaagtggcaccgagtcggtgc

SEQ ID NO: 687 - RNA, 111 nt, synthetic construct
  source 1..111: mol_type = other RNA
  modified_base 1..3: mod_base = OTHER; note = 2'-O-methylated nucleotide
  modified_base 109..111: mod_base = OTHER; note = 2'-O-methylated nucleotide
  modified_base 1..4: mod_base = OTHER; note = there is a 3'-phosphorothioate linkage between two adjoining nucleotides
  modified_base 108..111: mod_base = OTHER; note = there is a 3'-phosphorothioate linkage between two adjoining nucleotides
ctcttacctgtaccataaccgtttgagagctagggccctgaagaagggccctagcaagttcaaataaggctagtccgttatcaacttgaaaaagtggcaccgagtcggtgc

SEQ ID NO: 688 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
caagcaacttgtttgagagctaggccaacatgaggatcacccatgtctgcagggcctagcaagttcaaataaggctagtccgttatcaacttgaaaaagtggcaccgagtcggtgc

SEQ ID NO: 689 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
gtatctgtaggaggagaagtgtttgagagctagggccctgaagaagggccctagcaagttcaaataaggctagtccgttatcaacttgggccctgaagaagggcccaagtggcaccgagtcggtgc

SEQ ID NO: 690 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
gaggggctgcgtttgagagctaggccaacatgaggatcacccatgtctgcagggcctagcaagttcaaataaggctagtccgttatcaacttgaaaaagtggcaccgagtcggtgc

SEQ ID NO: 691 - RNA, 111 nt, synthetic construct
  source 1..111: mol_type = other RNA
  modified_base 1..3: mod_base = OTHER; note = 2'-O-methylated nucleotide
  modified_base 109..111: mod_base = OTHER; note = 2'-O-methylated nucleotide
  modified_base 1..4: mod_base = OTHER; note = there is a 3'-phosphorothioate linkage between two adjoining nucleotides
  modified_base 108..111: mod_base = OTHER; note = there is a 3'-phosphorothioate linkage between two adjoining nucleotides
ggagtctgagagatggagaggtttgagagctagggccctgaagaagggccctagcaagttcaaataaggctagtccgttatcaacttgaaaaagtggcaccgagtcggtgc

SEQ ID NO: 692 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
gctggggagagtttgagagctaggccaacatgaggatcacccatgtctgcagggcctagcaagttcaaataaggctagtccgttatcaacttgaaaaagtggcaccgagtcggtgc

SEQ ID NO: 693 - RNA, 111 nt, synthetic construct
  source 1..111: mol_type = other RNA
  modified_base 1..3: mod_base = OTHER; note = 2'-O-methylated nucleotide
  modified_base 109..111: mod_base = OTHER; note = 2'-O-methylated nucleotide
  modified_base 1..4: mod_base = OTHER; note = there is a 3'-phosphorothioate linkage between two adjoining nucleotides
  modified_base 108..111: mod_base = OTHER; note = there is a 3'-phosphorothioate linkage between two adjoining nucleotides
ggggttccagggcctgtctggtttgagagctagggccctgaagaagggccctagcaagttcaaataaggctagtccgttatcaacttgaaaaagtggcaccgagtcggtgc

SEQ ID NO: 694 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
ttgtccccttgtttgagagctaggccaacatgaggatcacccatgtctgcagggcctagcaagttcaaataaggctagtccgttatcaacttgaaaaagtggcaccgagtcggtgc

SEQ ID NO: 695 - RNA, 111 nt, synthetic construct
  source 1..111: mol_type = other RNA
  modified_base 1..3: mod_base = OTHER; note = 2'-O-methylated nucleotide
  modified_base 109..111: mod_base = OTHER; note = 2'-O-methylated nucleotide
  modified_base 1..4: mod_base = OTHER; note = there is a 3'-phosphorothioate linkage between two adjoining nucleotides
  modified_base 108..111: mod_base = OTHER; note = there is a 3'-phosphorothioate linkage between two adjoining nucleotides
ggacccagactagcagcaccgtttgagagctagggccctgaagaagggccctagcaagttcaaataaggctagtccgttatcaacttgaaaaagtggcaccgagtcggtgc

SEQ ID NO: 696 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
tcgtgcggccgtttgagagctaggccaacatgaggatcacccatgtctgcagggcctagcaagttcaaataaggctagtccgttatcaacttgaaaaagtggcaccgagtcggtgc

SEQ ID NO: 697 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
actcacgctggatagcctccgtttgagagctagggccctgaagaagggccctagcaagttcaaataaggctagtccgttatcaacttgggccctgaagaagggcccaagtggcaccgagtcggtgc

SEQ ID NO: 698 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
ggtgcagagcgtttgagagctaggccaacatgaggatcacccatgtctgcagggcctagcaagttcaaataaggctagtccgttatcaacttgaaaaagtggcaccgagtcggtgc

SEQ ID NO: 699 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
tcgatctatgaaaaagacaggtttgagagctagggccctgaagaagggccctagcaagttcaaataaggctagtccgttatcaacttgggccctgaagaagggcccaagtggcaccgagtcggtgc

SEQ ID NO: 700 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
cattttctcagtttgagagctaggccaacatgaggatcacccatgtctgcagggcctagcaagttcaaataaggctagtccgttatcaacttgaaaaagtggcaccgagtcggtgc

SEQ ID NO: 701 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
gtcatgcagcccttgcctgcgtttgagagctagggccctgaagaagggccctagcaagttcaaataaggctagtccgttatcaacttgggccctgaagaagggcccaagtggcaccgagtcggtgc

SEQ ID NO: 702 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
gactgggcagcggcccctgtgtttgagagctaggccaacatgaggatcacccatgtctgcagggcctagcaagttcaaataaggctagtccgttatcaacttgaaaaagtggcaccgagtcggtgc

SEQ ID NO: 703 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
cggcccctgtgtttgagagctaggccaacatgaggatcacccatgtctgcagggcctagcaagttcaaataaggctagtccgttatcaacttgaaaaagtggcaccgagtcggtgc

SEQ ID NO: 704 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
catgcagcccttgcctgctggtttgagagctagggccctgaagaagggccctagcaagttcaaataaggctagtccgttatcaacttgggccctgaagaagggcccaagtggcaccgagtcggtgc

SEQ ID NO: 705 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
ctgtggagcggactgggcaggtttgagagctaggccaacatgaggatcacccatgtctgcagggcctagcaagttcaaataaggctagtccgttatcaacttgaaaaagtggcaccgagtcggtgc

SEQ ID NO: 706 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
gactgggcaggtttgagagctaggccaacatgaggatcacccatgtctgcagggcctagcaagttcaaataaggctagtccgttatcaacttgaaaaagtggcaccgagtcggtgc

SEQ ID NO: 707 - RNA, 111 nt, synthetic construct
  source 1..111: mol_type = other RNA
  modified_base 1..3: mod_base = OTHER; note = 2'-O-methylated nucleotide
  modified_base 109..111: mod_base = OTHER; note = 2'-O-methylated nucleotide
  modified_base 1..4: mod_base = OTHER; note = there is a 3'-phosphorothioate linkage between two adjoining nucleotides
  modified_base 108..111: mod_base = OTHER; note = there is a 3'-phosphorothioate linkage between two adjoining nucleotides
ctcaccagattcccgaaggtgtttgagagctagggccctgaagaagggccctagcaagttcaaataaggctagtccgttatcaacttgaaaaagtggcaccgagtcggtgc

SEQ ID NO: 708 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
cagactactcgtttgagagctaggccaacatgaggatcacccatgtctgcagggcctagcaagttcaaataaggctagtccgttatcaacttgaaaaagtggcaccgagtcggtgc

SEQ ID NO: 709 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
actccagttcctgacggctggtttgagagctagggccctgaagaagggccctagcaagttcaaataaggctagtccgttatcaacttgggccctgaagaagggcccaagtggcaccgagtcggtgc

SEQ ID NO: 710 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
tgcctcttggaagacagagagtttgagagctaggccaacatgaggatcacccatgtctgcagggcctagcaagttcaaataaggctagtccgttatcaacttgaaaaagtggcaccgagtcggtgc

SEQ ID NO: 711 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
aagacagagagtttgagagctaggccaacatgaggatcacccatgtctgcagggcctagcaagttcaaataaggctagtccgttatcaacttgaaaaagtggcaccgagtcggtgc

SEQ ID NO: 712 - RNA, 126 nt, synthetic construct
  source 1..126: mol_type = other RNA
  modified_base 1..3: mod_base = OTHER; note = there is a 3'-phosphorothioate linkage between two adjoining nucleotides
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
acctacaggagtacctgacggtttgagagctagggccctgaagaagggccctagcaagttcaaataaggctagtccgttatcaacttgggccctgaagaagggcccaagtggcaccgagtcggtgc

SEQ ID NO: 713 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
cggagttggggaaacaatacgtttgagagctaggccaacatgaggatcacccatgtctgcagggcctagcaagttcaaataaggctagtccgttatcaacttgaaaaagtggcaccgagtcggtgc

SEQ ID NO: 714 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
gaaacaatacgtttgagagctaggccaacatgaggatcacccatgtctgcagggcctagcaagttcaaataaggctagtccgttatcaacttgaaaaagtggcaccgagtcggtgc

SEQ ID NO: 715 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
gagggtccagatgcccagcagtttgagagctagggccctgaagaagggccctagcaagttcaaataaggctagtccgttatcaacttgggccctgaagaagggcccaagtggcaccgagtcggtgc

SEQ ID NO: 716 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
gggcaggagagtttgagagctaggccaacatgaggatcacccatgtctgcagggcctagcaagttcaaataaggctagtccgttatcaacttgaaaaagtggcaccgagtcggtgc

SEQ ID NO: 717 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
ttcacctgcccaaggaaaaagtttgagagctagggccctgaagaagggccctagcaagttcaaataaggctagtccgttatcaacttgggccctgaagaagggcccaagtggcaccgagtcggtgc

SEQ ID NO: 718 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
gcacgcagtcgtttgagagctaggccaacatgaggatcacccatgtctgcagggcctagcaagttcaaataaggctagtccgttatcaacttgaaaaagtggcaccgagtcggtgc

SEQ ID NO: 719 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
ctccactgctggcgccacctgtttgagagctagggccctgaagaagggccctagcaagttcaaataaggctagtccgttatcaacttgggccctgaagaagggcccaagtggcaccgagtcggtgc

SEQ ID NO: 720 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
cgcgccaggagtttgagagctaggccaacatgaggatcacccatgtctgcagggcctagcaagttcaaataaggctagtccgttatcaacttgaaaaagtggcaccgagtcggtgc

SEQ ID NO: 721 - RNA, 126 nt, synthetic construct
  source 1..126: mol_type = other RNA
  modified_base 1..3: mod_base = OTHER; note = 2'-O-methylated nucleotide
  modified_base 124..126: mod_base = OTHER; note = 2'-O-methylated nucleotide
  modified_base 1..4: mod_base = OTHER; note = there is a 3'-phosphorothioate linkage between two adjoining nucleotides
  modified_base 123..126: mod_base = OTHER; note = there is a 3'-phosphorothioate linkage between two adjoining nucleotides
ttttccagaatactgaaacagtttgagagctagggccctgaagaagggccctagcaagttcaaataaggctagtccgttatcaacttgggccctgaagaagggcccaagtggcaccgagtcggtgc

SEQ ID NO: 722 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
tttcactactgtttgagagctaggccaacatgaggatcacccatgtctgcagggcctagcaagttcaaataaggctagtccgttatcaacttgaaaaagtggcaccgagtcggtgc

SEQ ID NO: 723 - RNA, 111 nt, synthetic construct
  source 1..111: mol_type = other RNA
  modified_base 1..3: mod_base = OTHER; note = 2'-O-methylated nucleotide
  modified_base 109..111: mod_base = OTHER; note = 2'-O-methylated nucleotide
  modified_base 1..4: mod_base = OTHER; note = there is a 3'-phosphorothioate linkage between two adjoining nucleotides
  modified_base 108..111: mod_base = OTHER; note = there is a 3'-phosphorothioate linkage between two adjoining nucleotides
gctaaccaataaggggtggcgtttgagagctagggccctgaagaagggccctagcaagttcaaataaggctagtccgttatcaacttgaaaaagtggcaccgagtcggtgc

SEQ ID NO: 724 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
gaaacgggaagtttgagagctaggccaacatgaggatcacccatgtctgcagggcctagcaagttcaaataaggctagtccgttatcaacttgaaaaagtggcaccgagtcggtgc

SEQ ID NO: 725 - RNA, 111 nt, synthetic construct
  source 1..111: mol_type = other RNA
  modified_base 1..3: mod_base = OTHER; note = 2'-O-methylated nucleotide
  modified_base 109..111: mod_base = OTHER; note = 2'-O-methylated nucleotide
  modified_base 1..4: mod_base = OTHER; note = there is a 3'-phosphorothioate linkage between two adjoining nucleotides
  modified_base 108..111: mod_base = OTHER; note = there is a 3'-phosphorothioate linkage between two adjoining nucleotides
attatacctgccatgccgtagtttgagagctagggccctgaagaagggccctagcaagttcaaataaggctagtccgttatcaacttgaaaaagtggcaccgagtcggtgc

SEQ ID NO: 726 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
aaatattagtgtttgagagctaggccaacatgaggatcacccatgtctgcagggcctagcaagttcaaataaggctagtccgttatcaacttgaaaaagtggcaccgagtcggtgc

SEQ ID NO: 727 - RNA, 111 nt, synthetic construct
  source 1..111: mol_type = other RNA
  modified_base 1..3: mod_base = OTHER; note = 2'-O-methylated nucleotide
  modified_base 109..111: mod_base = OTHER; note = 2'-O-methylated nucleotide
  modified_base 1..4: mod_base = OTHER; note = there is a 3'-phosphorothioate linkage between two adjoining nucleotides
  modified_base 108..111: mod_base = OTHER; note = there is a 3'-phosphorothioate linkage between two adjoining nucleotides
tgaacaggaaataacctatggtttgagagctagggccctgaagaagggccctagcaagttcaaataaggctagtccgttatcaacttgaaaaagtggcaccgagtcggtgc

SEQ ID NO: 728 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
gaaaacctaagtttgagagctaggccaacatgaggatcacccatgtctgcagggcctagcaagttcaaataaggctagtccgttatcaacttgaaaaagtggcaccgagtcggtgc

SEQ ID NO: 729 - RNA, 111 nt, synthetic construct
  source 1..111: mol_type = other RNA
  modified_base 1..3: mod_base = OTHER; note = 2'-O-methylated nucleotide
  modified_base 109..111: mod_base = OTHER; note = 2'-O-methylated nucleotide
  modified_base 1..4: mod_base = OTHER; note = there is a 3'-phosphorothioate linkage between two adjoining nucleotides
  modified_base 108..111: mod_base = OTHER; note = there is a 3'-phosphorothioate linkage between two adjoining nucleotides
taaatctgcagggagagaaagtttgagagctagggccctgaagaagggccctagcaagttcaaataaggctagtccgttatcaacttgaaaaagtggcaccgagtcggtgc

SEQ ID NO: 730 - RNA, 116 nt, synthetic construct
  source 1..116: mol_type = other RNA
  modified_base 1..3: mod_base = OTHER; note = 2'-O-methylated nucleotide
  modified_base 114..116: mod_base = OTHER; note = 2'-O-methylated nucleotide
  modified_base 1..4: mod_base = OTHER; note = there is a 3'-phosphorothioate linkage between two adjoining nucleotides
  modified_base 113..116: mod_base = OTHER; note = there is a 3'-phosphorothioate linkage between two adjoining nucleotides
gataattcccgtttgagagctaggccaacatgaggatcacccatgtctgcagggcctagcaagttcaaataaggctagtccgttatcaacttgaaaaagtggcaccgagtcggtgc

SEQ ID NO: 731 - RNA, 3585 nt, synthetic construct
  source 1..3585: mol_type = other RNA
gggggacagatcgcctggagacgccatccacgctgttttgacctccatagaagacaccgggaccgatccagcctccgcggccgggaacggtgcattggaacgcggattccccgtgccaagagtgactcaccgtccttgacacggccaccatggcctctaacttcacccagtttgtgctggtggacaatggaggaaccggcgatgtgacagtggcaccatccaactttgccaatggcatcgccgagtggatcagctccaactctaggagccaggcctacaaggtgacctgcagcgtgcggcagtctagcgcccagaatagaaagtacacaatcaaggtggaggtgccaaagggagcatggcgcagctatctgaacatggagctgaccatccccatcttcgccacaaattccgactgtgagctgatcgtgaaggccatgcagggcctgctgaaggatggcaaccccatcccttctgccatcgccgccaatagcggcatctactctggaggatctagcggaggcagctctggcagcgagacaccaggaacaagcgagtcagcaacaccagagagcagtggcggcagcagcggcggcagcagcggcggatccaagcggcccgccgccaccaagaaggccggccaggccaagaagaagaagatgaccaacctgtctgacatcatcgagaaggagacaggcaagcagctggtcatccaggagagcatcctgatgctgcccgaagaagtcgaagaagtgatcggaaacaagcctgagagcgatatcctggtccataccgcctacgacgagagtaccgacgaaaatgtgatgctgctgacatccgacgccccagagtataagccctgggctctggtcatccaggattccaacggagagaacaaaatcaaaatgctgtctggaggcagcatgggaccattttgcctgggctgttctcaccggaagtgctatagccccatcagaaacctgatcagccaggagacattcaagtttcacttcaagaatctgggctacgccaagggccggaaggataccttcctgtgctatgaggtgacaagaaaggactgtgatagccccgtgtccctgcaccacggcgtgtttaagaacaaggacaatatccacgccgagatctgttttctgtactggttccacgataaggtgctgaaggtgctgtcccccagggaggagttcaagatcacctggtatatgtcttggagcccttgcttcgagtgtgccgagcagatcgtgcgctttctggccacacaccacaacctgagcctggacatctttagctcccggctgtacaacgtgcaggatcctgagacacagcagaatctgtgcagactggtgcaggagggagcacaggtggcagcaatggacctgtatgagttcaagaagtgttggaagaagtttgtggataacggcggccggagattccggccgtggaagagactgctgaccaacttccggtaccaggacagcaagctgcaggagatcctgcgcccttgctatatctccgtgccatctagcgagaatctgtacttccagagcatgtcctctagcaccctgtctaatatctgcctgaccaagggcctgcccgagacaaggttctgggtggagggccggagaatggaccctctgagcgaggaggagttttactcccagttctataaccagagggtgaagcacctgtgctactatcaccgcatgaagccttacctgtgctatcagctggagcagttcaatggacaggcaccactgaagggatgcctgctgtccgagaagggcaagcagcacgccgagatcctgtttctggataagatcagatctatggagctgagccaggtgaccatcacatgttacctgacatggtccccatgcccaaactgtgcatggcagctggcagccttcaagagggaccgcccagatctgatcctgcacatctacacctctaggctgtattttcactggaagcgccccttccagaagggcctgtgctccctgtggcagtctggcatcctggtggacgtgatggatctgccccagtttaccgactgttggacaaacttcgtgaatcctaagcggccattttggccctggaagggcctggagatcatcagcaggcgcacacagcggagactgaggcgcatcaaggagtcctggggcctgcaggacctggtgaacgattttggcaatctgcagctgggaccccctatgagcaagcggcccgccgccaccaagaaggccggccaggccaagaagaagaaggctactaacttcagcctgctgaagcaggctggggacgtggaggagaaccctggacctatgggcaacgccagaacaagacggcgggagcgcagagccgagaagcaggcccaatggaaagccgctaattccggcgggagcagtggcggatccagtgggtccgaaacccctggcacaagcgaaagcgctacccctgagtccagcggagggtcctcaggggaattcatgaaaagcatgtctagcatggtgtctgacacaagctgtaccttcccctcctctgatggcatcttttggaagcactggattcagacaaaggacggccagtgtggctcccctctggtgtctaccagggatggcttcatcgtgggcatccacagcgcctccaacttcacaaacaccaacaactactttacatccgtgcctaagaacttcatggagctgctgaccaatcaggaggcacagcagtgggtgtctggatggcgcctgaacgccgatagcgtgctgtggggcggccacaaggtgtttatggtgaagccagaggagcccttccagcctgtgaaggaggccacccagaagcggcccgccgccaccaagaaggccggccaggccaagaagaagaaggagggcagaggaagtctgctaacatgcggtgacgtcgaggagaatcctggcccaatgggcgagtccctgtttaagggcccacgggactacaaccccatcagctccacaatctgccacctgaccaatgagagcgatggccacaccacatccctgtatggcatcggcttcggccccttcatcatcacaaacaagcacctgtttcggagaaacaatggcaccctgctggtgcagtctctgcacggcgtgttcaaggtgaagaataccacaaccctgcagcagcacctgatcgacggaagggatatgatcatcatcagaatgcctaaggacttccccccttttccacagaagctgaagtttcgggagccacagagggaggagaggatctgcctggtgacaaccaacttccagacactcgagaagcggcccgccgccaccaagaaggccggccaggccaagaagaagaagtaatgagctccgggtggcatccctgtgacccctccccagtgcctctcctggccctggaagttgccactccagtgcccaccagccttgtcctaataaaattaagttgcatcaagctgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 732 - RNA, 4593 nt, synthetic construct
  source 1..4593: mol_type = other RNA
gggggacagatcgcctggagacgccatccacgctgttttgacctccatagaagacaccgggaccgatccagcctccgcggccgggaacggtgcattggaacgcggattccccgtgccaagagtgactcaccgtccttgacacggccaccatggcccctgccgccaagcgggtcaagctcgacggtatccacggagtcccagcagctatggacaagaagtacagcatcggcctggccatcggcaccaacagcgtgggctgggccgtgatcaccgacgagtacaaggtgcccagcaagaagttcaaggtgctgggcaacaccgaccggcacagcatcaagaagaacctgatcggcgccctgctgttcgacagcggcgagaccgccgaggccacccggctgaagcggaccgcccggcggcggtacacccggcggaagaaccggatctgctacctgcaggagatcttcagcaacgagatggccaaggtggacgacagcttcttccaccggctggaggagagcttcctggtggaggaggacaagaagcacgagcggcaccccatcttcggcaacatcgtggacgaggtggcctaccacgagaagtaccccaccatctaccacctgcggaagaagctggtggacagcaccgacaaggccgacctgcggctgatctacctggccctggcccacatgatcaagttccggggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccggctgagcaagagccggcggctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgggtgaacaccgagatcaccaaggcccccctgagcgccagcatgatcaagcggtacgacgagcaccaccaggacctgaccctgctgaaggccctggtgcggcagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgggaggacctgctgcggaagcagcggaccttcgacaacggcagcatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaacagccggttcgcctggatgacccgaaagagcgaggagaccatcaccccctggaacttcgaggaggtggtggacaagggcgccagcgcccagagcttcatcgagcggatgaccaacttcgacaagaacctgcccaacgagaaggtgctgcccaagcacagcctgctgtacgagtacttcaccgtgtacaacgagctgaccaaggtgaagtacgtgaccgagggcatgcggaagcccgccttcctgagcggcgagcagaagaaggccatcgtggacctgctgttcaagaccaaccggaaggtgaccgtgaagcagctgaaggaggactacttcaagaagatcgagtgcttcgacagcgtggagatcagcggcgtggaggaccggttcaacgccagcctgggcacctaccacgacctgctgaagatcatcaaggacaaggacttcctggacaacgaggagaacgaggacatcctggaggacatcgtgctgaccctgaccctgttcgaggaccgggagatgatcgaggagcggctaaagacctacgcccacctgttcgacgacaaggtgatgaagcagctgaagcggcggcggtacaccggctggggccggctgagccggaagctgatcaacggcatccgggacaagcagagcggcaagaccatcctggacttcctcaagagcgacggcttcgccaaccggaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaacagccgggagcggatgaagcggatcgaggagggcatcaaggagctgggcagccagatcctgaaggagcaccccgtggagaacacccagctgcagaacgagaagctgtacctgtactacctgcagaacggccgggacatgtacgtggaccaggagctggacatcaaccggctgagcgactacgacgtggaccacatcgtgccccagagcttcctgaaggacgacagcatcgacaacaaggtgctgacccggagcgacaagaaccggggcaagagcgacaacgtgcccagcgaggaggtggtgaagaagatgaagaactactggcggcagctgctgaacgccaagctgatcacccagcggaagttcgacaacctgaccaaggccgagcggggcggcctgagcgagctggacaaggccggcttcatcaagcggcagctggtggagacccggcagatcaccaagcacgtggcccagatcctggacagccggatgaacaccaagtacgacgagaacgacaagctgatccgggaggtgaaggtgatcaccctcaagagcaagctggtgagcgacttccggaaggacttccagttctacaaggtgcgggagatcaacaactaccaccacgcccacgacgcctacctgaacgccgtggtgggcaccgccctgatcaagaagtaccccaagctggagagcgagttcgtgtacggcgactacaaggtgtacgacgtgcggaagatgatcgccaagagcgagcaggagatcggcaaggccaccgccaagtacttcttctacagcaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgagcatgccccaggtgaacatcgtgaaaaagaccgaggtgcagaccggcggcttcagcaaggagagcatcctgcccaagcggaacagcgacaagctgatcgcccggaagaaggactgggaccccaagaagtacggcggcttcgacagccccaccgtggcctacagcgtgctggtggtggccaaggtggagaagggcaagagcaagaagctcaagagcgtgaaggagctgctgggcatcaccatcatggagcggagcagcttcgagaagaaccccatcgacttcctggaggccaagggctacaaggaggtgaagaaggacctgatcatcaagctgcccaagtacagcctgttcgagctggagaacggccggaagcggatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgggtgatcctggccgacgccaacctggacaaggtgctgagcgcctacaacaagcaccgggacaagcccatccgggagcaggccgagaacatcatccacctgttcaccctgaccaacctgggcgcccccgccgccttcaagtacttcgacaccaccatcgaccggaagcggtacaccagcaccaaggaggtgctggacgccaccctgatccaccagagcatcaccggcctgtacgagacccggatcgacctgagccagctgggcggcgacagcggcggcagcaagcggcccgccgccaccaagaaggccggccaggccaagaagaagaagtaatgagctccgggtggcatccctgtgacccctccccagtgcctctcctggccctggaagttgccactccagtgcccaccagccttgtcctaataaaattaagttgcatcaagctgcggccgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 733 - protein, 1368 aa, Streptococcus pyogenes [sequence omitted]

SEQ ID NO: 734 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 735 - protein, 1368 aa, synthetic construct; identical to SEQ ID NO: 146

SEQ ID NO: 736 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 737 - protein, 1629 aa, synthetic construct
MNFKILPIAIDLGVKNTGVFSAFYQKGTSLERLDNKNGKVYELSKDSYTLLMNNRTARRHQRRGIDRKQLVKRLFKLIWTEQLNLEWDKDTQQAISFLFNRRGFSFITDGYSPEYLNIVPEQVKAILMDIFDDYNGEDDLDSYLKLATEQESKISEIYNKLMQKILEFKLMKLCTDIKDDKVSTKTLKEITSYEFELLADYLANYSESLKTQKFSYTDKQGNLKELSYYHHDKYNIQEFLKRHATINDRILDTLLTDDLDIWNFNFEKFDFDKNEEKLQNQEDKDHIQAHLHHFVFAVNKIKSEMASGGRHRSQYFQEITNVLDENNHQEGYLKNFCENLHNKKYSNLSVKNLVNLIGNLSNLELKPLRKYFNDKIHAKADHWDEQKFTETYCHWILGEWRVGVKDQDKKDGAKYSYKDLCNELKQKVTKAGLVDFLLELDPCRTIPPYLDNNNRKPPKCQSLILNPKFLDNQYPNWQQYLQELKKLQSIQNYLDSFETDLKVLKSSKDQPYFVEYKSSNQQIASGQRDYKDLDARILQFIFDRVKASDELLLNEIYFQAKKLKQKASSELEKLESSKKLDEVIANSQLSQILKSQHTNGIFEQGTFLHLVCKYYKQRQRARDSRLYIMPEYRYDKKLHKYNNTGRFDDDNQLLTYCNHKPRQKRYQLLNDLAGVLQVSPNFLKDKIGSDDDLFISKWLVEHIRGFKKACEDSLKIQKDNRGLLNHKINIARNTKGKCEKEIFNLICKIEGSEDKKGNYKHGLAYELGVLLFGEPNEASKPEFDRKIKKFNSIYSFAQIQQIAFAERKGNANTCAVCSADNAHRMQQIKITEPVEDNKDKIILSAKAQRLPAIPTRIVDGAVKKMATILAKNIVDDNWQNIKQVLSAKHQLHIPIITESNAFEFEPALADVKGKSLKDRRKKALERISPENIFKDKNNRIKEFAKGISAYSGANLTDGDFDGAKEELDHIIPRSHKKYGTLNDEANLICVTRGDNKNKGNRIFCLRDLADNYKLKQFETTDDLEIEKKIADTIWDANKKDFKFGNYRSFINLTPQEQKAFRHALFLADENPIKQAVIRAINNRNRTFVNGTQRYFAEVLANNIYLRAKKENLNTDKISFDYFGIPTIGNGRGIAEIRQLYEKVDSDIQAYAKGDKPQASYSHLIDAMLAFCIAADEHRNDGSIGLEIDKNYSLYPLDKNTGEVFTKDIFSQIKITDNEFSDKKLVRKKAIEGFNTHRQMTRDGIYAENYLPILIHKELNEVRKGYTWKNSEEIKIFKGKKYDIQQLNNLVYCLKFVDKPISIDIQISTLEELRNILTTNNIAATAEYYYINLKTQKLHEYYIENYNTALGYKKYSKEMEFLRSLAYRSERVKIKSIDDVKQVLDKDSNFIIGKITLPFKKEWQRLYREWQNTTIKDDYEFLKSFFNVKSITKLHKKVRKDFSLPISTNEGKFLVKRKTWDNNFIYQILNDSDSRADGTKPFIPAFDISKNEIVEAIIDSFTSKNIFWLPKNIELQKVDNKNIFAIDTSKWFEVETPSDLRDIGIATIQYKIDNNSRPKVRVKLDYVIDDDSKINYFMNHSLLKSRYPDKVLEILKQSTIIEFESSGFNKTIKEMLGMKLAGIYNETSNN

SEQ ID NO: 738 - protein, 1122 aa, synthetic construct
MGSDLVLGLDIGIGSVGVGILNKVTGEIIHKNSRIFPAAQAENNLVRRTNRQGRRLARRKKHRRVRLNRLFEESGLITDFTKISINLNPYQLRVKGLTDELSNEELFIALKNMVKHRGISYLDDASDDGNSSVGDYAQIVKENSKQLETKTPGQIQLERYQTYGQLRGDFTVEKDGKKHRLINVFPTSAYRSEALRILQTQQEFNPQITDEFINRYLEILTGKRKYYHGPGNEKSRTDYGRYRTSGETLDNIFGILIGKCTFYPDEFRAAKASYTAQEFNLLNDLNNLTVPTETKKLSKEQKNQIINYVKNEKAMGPAKLFKYIAKLLSCDVADIKGYRIDKSGKAEIHTFEAYRKMKTLETLDIEQMDRETLDKLAYVLTLNTEREGIQEALEHEFADGSFSQKQVDELVQFRKANSSIFGKGWHNFSVKLMMELIPELYETSEEQMTILTRLGKQKTTSSSNKTKYIDEKLLTEEIYNPVVAKSVRQAIKIVNAAIKEYGDFDNIVIEMARETNEDDEKKAIQKIQKANKDEKDAAMLKAANQYNGKAELPHSVFHGHKQLATKIRLWHQQGERCLYTGKTISIHDLINNSNQFEVDHILPLSITFDDSLANKVLVYATANQEKGQRTPYQALDSMDDAWSFRELKAFVRESKTLSNKKKEYLLTEEDISKFDVRKKFIERNLVDTRYASRVVLNALQEHFRAHKIDTKVSVVRGQFTSQLRRHWGIEKTRDTYHHHAVDALIIAASSQLNLWKKQKNTLVSYSEDQLLDIETGELISDDEYKESVFKAPYQHFVDTLKSKEFEDSILFSYQVDSKFNRKISDATIYATRQAKVGKDKADETYVLGKIKDIYTQDGYDAFMKIYKKDKSKFLMYRHDPQTFEKVIEPILENYPNKQINEKGKEVPCNPFLKYKEEHGYIRKYSKKGNGPEIKSLKYYDSKLGNHIDITPKDSNNKVVLQSVSPWRADVYFNKTTGKYEILGLKYADLQFEKGTGTYKISQEKYNDIKKKEGVDSDSEFKFTLYKNDLLLVKDTETKEQQLFRFLSRTMPKQKHYVELKPYDKQKFEGGEALIKVLGNVANSGQCKKGLGKSNISIYKVRTDVLGNQHIIKNEGDKPKLDF

SEQ ID NO: 739 - protein, 1388 aa, synthetic construct
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 740 - protein, 1082 aa, synthetic construct
MAAFKPNSINYILGLDIGIASVGWAMVEIDEEENPIRLIDLGVRVFERAEVPKTGDSLAMARRLARSVRRLTRRRAHRLLRTRRLLKREGVLQAANFDENGLIKSLPNTPWQLRAAALDRKLTPLEWSAVLLHLIKHRGYLSQRKNEGETADKELGALLKGVAGNAHALQTGDFRTPAELALNKFEKESGHIRNQRSDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTLEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 741 - protein, 1053 aa, synthetic construct
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 742 - protein, 1307 aa, synthetic construct
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 743 - protein, 1229 aa, synthetic construct
MAASKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGAAGYKSLFKKDIIETILPEAADDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNAKTKQALPKFKPLYKQVLSDRESLSFYGEGYTSDEEVLEVFRNTLNKNSEIFSSIKKLEKLFKNFDEYSSAGIFVKNGPAISTISKDIFGEWNLIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFAAAKKNNVFAWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVK

SEQ ID NO: 744 - protein, 1300 aa, synthetic construct
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 745 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 746 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFESPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 747 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKEYRSTKEVLDATLIHQSITGLYETRIDLSQLGGD

SEQ ID NO: 748 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFVSPTVAYSVLVVAKVEKGKSKKLKSVKELLGITIMERSSFEKNPIDFLEAKGYKEVKKDLIIKLPKYSLFELENGRKRMLASARFLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPRAFKYFDTTIDRKVYRSTKEVLDATLIHQSITGLYETRIDLSQLGGD

SEQ ID NO: 749 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDTKLQLSKDTYDDDLDNLLAQIGDQYADLFLAAKNLSDAILLSDILRVNTEITKAPLSASMIKLYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQEDFYPFLKDNREKIEKILTFRIPYYVGPLARGNSRFAWMTRKSEETITPWNFEKVVDKGASAQSFIERMTNFDKNLPNEKVLPKHSLLYEYFTVYNELTKVKYVTEGMRKPAFLSGDQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFIQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 750 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLADDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPALESEFVYGDYKVYDVRKMIAKSEQEIGKATAKYFFYSNIMNFFKTEITLANGEIRKAPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 751 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTAFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGALSRKLINGIRDKQSGKTILDFLKSDGFANRNFMALIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRAITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 752 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRAFAALIADDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 753 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANANFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 754 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPASLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEIARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNANLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 755 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKQYRSTKEVLDATLIHQSITGLYETRIDLSQLGGD

SEQ ID NO: 756 - protein, 1368 aa, synthetic construct
MDKKYSIGLDIGTNSVGWAVITDEYKVPSKKFKVLGNTDRHSIKKNLIGALLFDSGETAERTRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESIRPKRNSDKLIARKKDWDPKKYGGFLWPTVAYSVLVVAKVEKGKSKKLKSVKELLGITIMERSSFEKNPIDFLEAKGYKEVKKDLIIKLPKYSLFELENGRKRMLASAKQLQKGNELALPSKYVNFLYLASHYEKLKGSPEDNEQKQLFVEQHKHYLDEIIEQISEFSKRVILADANLDKVLSAYNKHRDKPIREQAENIIHLFTLTRLGAPRAFKYFDTTIDPKQYRSTKEVLDATLIHQSITGLYETRIDLSQLGGD

SEQ ID NO: 757 - protein, 1054 aa, synthetic construct
MGKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRKLINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYKNDLIKINGELYRVIGVNNDLLNRIEVNMIDITYREYLENMNDKRPPHIIKTIASKTQSIKKYSTDILGNLYEVKSKKHPQIIKKG

SEQ ID NO: 758 - protein, 984 aa, synthetic construct
MARILAFDIGISSIGWAFSENDELKDCGVRIFTKVENPKTGESLALPRRLARSARKRLARRKARLNHLKHLIANEFKLNYEDYQSFDESLAKAYKGSLISPYELRFRALNELLSKQDFARVILHIAKRRGYDDIKNSDDKEKGAILKAIKQNEEKLANYQSVGEYLYKEYFQKFKENSKEFTNVRNKKESYERCIAQSFLKDELKLIFKKQREFGFSFSKKFEEEVLSVAFYKRALKDFSHLVGNCSFFTDEKRAPKNSPLAFMFVALTRIINLLNNLKNTEGILYTKDDLNALLNEVLKNGTLTYKQTKKLLGLSDDYEFKGEKGTYFIEFKKYKEFIKALGEHNLSQDDLNEIAKDITLIKDEIKLKKALAKYDLNQNQIDSLSKLEFKDHLNISFKALKLVTPLMLEGKKYDEACNELNLKVAINEDKKDFLPAFNETYYKDEVTNPVVLRAIKEYRKVLNALLKKYGKVHKINIELAREVGKNHSQRAKIEKEQNENYKAKKDAELECEKLGLKINSKNILKLRLFKEQKEFCAYSGEKIKISDLQDEKMLEIDHIYPYSRSFDDSYMNKVLVFTKQNQEKLNQTPFEAFGNDSAKWQKIEVLAKNLPTKKQKRILDKNYKDKEQKNFKDRNLNDTRYIARLVLNYTKDYLDFLPLSDDENTKLNDTQKGSKVHVEAKSGMLTSALRHTWGFSAKDRNNHLHHAIDAVIIAYANNSIVKAFSDFKKEQESNSAELYAKKISELDYKNKRKFFEPFSGFRQKVLDKIDEIFVSKPERKKPSGALHEETFRKEEEFYQSYGGKEGVLKALELGKIRKVNGKIVKNGDMFRVDIFKHKKTNKFYAVPIYTMDFALKVLPNKAVARSKKGEIKDWILMDENYEFCFSLYKDSLILIQTKDMQEPEFVYYNAFTSSTVSLIVSKHDNKFETLSKNQKILFKNANEKEVIAKSIGIQNLKVFEKYIVSALGEVTKAEFRQREDFKK

SEQ ID NO: 759 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTAAYSVLVVAKVEKGKSKKLKSVKELLGITIMERSSFEKNPIGFLEAKGYKEVKKDLIIKLPKYSLFELENGRKRMLASAGVLHKGNELALPSKYVNFLYLASHYEKLKGSPEDNEQKQLFVEQHKHYLDEIIEQISEFSKRVILADANLDKVLSAYNKHRDKPIREQAENIIHLFTLTNLGVPAAFKYFDTTIDKKRYTSTKEVLDATLIHQSITGLYETRIDLSQLGGD

SEQ ID NO: 760 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIDFLEAKGYKEVKKDLIIKLPKYSLFELENGRKRMLASAGVLQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTINRKQYNTTKEVLDATLIRQSITGLYETRIDLSQLGGD

SEQ ID NO: 761 - protein, 1368 aa, synthetic construct
MDKKYSIGLDIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMVKRYDEHHQDLTLLKALVRQQLPEKYKEIFFDQSKNGYAGYIDGGASQEEFYKFIKPILEKMDGTEELLVKLNREDLLRKQRTFDNGIIPHQIHLGELHAILRRQGDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRLRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGGHKPENIVIEMARENQTTQKGQKNSRERMKRIEEGIKELGSQILKEHPVENTQLQNEKLYLYYLQNGRDMYVDQELDINRLSDYDVDHIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKGNSDKLIARKKDWDPKKYGGFNSPTVAYSVLVVAKVEKGKSKKLKSVKELLGITIMERSSFEKNPIGFLEAKGYKEVKKDLIIKLPKYSLFELENGRKRMLASASVLHKGNELALPSKYVNFLYLASHYEKLKGSSEDNKQKQLFVEQHKHYLDEIIEQISEFSKRVILADANLDKVLSAYNKHRDKPIREQAENIIHLFTLTNLGASAAFKYFDTTIGRKLYTSTKEVLDATLIHQSITGLYETRIDLSQLGGD

SEQ ID NO: 762 - protein, 1250 aa, synthetic construct
MQTLFENFTNQYPVSKTLRFELIPQGKTKDFIEQKGLLKKDEDRAEKYKKVKNIIDEYHKDFIEKSLNGLKLDGLEKYKTLYLKQEKDDKDKKAFDKEKENLRKQIANAFRNNEKFKTLFAKELIKNDLMSFACEEDKKNVKEFEAFTTYFTGFHQNRANMYVADEKRTAIASRLIHENLPKFIDNIKIFEKMKKEAPELLSPFNQTLKDMKDVIKGTTLEEIFSLDYFNKTLTQSGIDIYNSVIGGRTPEEGKTKIKGLNEYINTDFNQKQTDKKKRQPKFKQLYKQILSDRQSLSFIAEAFKNDTEILEAIEKFYVNELLHFSNEGKSTNVLDAIKNAVSNLESFNLTKMYFRSGASLTDVSRKVFGEWSIINRALDNYYATTYPIKPREKSEKYEERKEKWLKQDFNVSLIQTAIDEYDNETVKGKNSGKVIADYFAKFCDDKETDLIQKVNEGYIAVKDLLNTPCPENEKLGSNKDQVKQIKAFMDSIMDIMHFVRPLSLKDTDKEKDETFYSLFTPLYDHLTQTIALYNKVRNYLTQKPYSTEKIKLNFENSTLLGGWDLNKETDNTAIILRKDNLYYLGIMDKRHNRIFRNVPKADKKDFCYEKMVYKLLPGANKMLPKVFFSQSRIQEFTPSAKLLENYANETHKKGDNFNLNHCHKLIDFFKDSINKHEDWKNFDFRFSATSTYADLSGFYHEVEHQGYKISFQSVADSFIDDLVNEGKLYLFQIYNKDFSPFSKGKPNLHTLYWKMLFDENNLKDVVYKLNGEAEVFYRKKSIAEKNTTIHKANESIINKNPDNPKATSTFNYDIVKDKRYTIDKFQFHIPITMNFKAEGIFNMNQRVNQFLKANPDINIIGIDRGERHLLYYALINQKGKILKQDTLNVIANEKQKVDYHNLLDKKEGDRATARQEWGVIETIKELKEGYLSQVIHKLTDLMIENNAIIVMEDLNFGFKRGRQKVEKQVYQKFEKMLIDKLNYLVDKNKKANELGGLLNAFQLANKFESFQKMGKQNGFIFYVPAWNTSKTDPATGFIDFLKPRYENLNQAKDFFEKFDSIRLNSKADYFEFAFDFKNFTEKADGGRTKWTVCTTNEDRYAWNRALNNNRGSQEKYDITAELKSLFDGKVDYKSGKDLKQQIASQESADFFKALMKNLSITLSLRHNNGEKGDNEQDYILSPVADSKGRFFDSRKADDDMPKNADANGAYHIALKGLWCLEQISKTDDLKKVKLAISNKEWLEFVQTLKG

SEQ ID NO: 763 - protein, 1260 aa, synthetic construct
MDSLKDFTNLYPVSKTLRFELKPVGKTLENIEKAGILKEDEHRAESYRRVKKIIDTYHKVFIDSSLENMAKMGIENEIKAMLQSFCELYKKDHRTEGEDKALDKIRAVLRGLIVGAFTGVCGRRENTVQNEKYESLFKEKLIKEILPDFVLSTEAESLPFSVEEATRSLKEFDSFTSYFAGFYENRKNIYSTKPQSTAIAYRLIHENLPKFIDNILVFQKIKEPIAKELEHIRADFSAGGYIKKDERLEDIFSLNYYIHVLSQAGIEKYNALIGKIVTEGDGEMKGLNEHINLYNQQRGREDRLPLFRPLYKQILSDREQLSYLPESFEKDEELLRALKEFYDHIAEDILGRTQQLMTSISEYDLSRIYVRNDSQLTDISKKMLGDWNAIYMARERAYDHEQAPKRITAKYERDRIKALKGEESISLANLNSCIAFLDNVRDCRVDTYLSTLGQKEGPHGLSNLVENVFASYHEAEQLLSFPYPEENNLIQDKDNVVLIKNLLDNISDLQRFLKPLWGMGDEPDKDERFYGEYNYIRGALDQVIPLYNKVRNYLTRKPYSTRKVKLNFGNSQLLSGWDRNKEKDNSCVILRKGQNFYLAIMNNRHKRSFENKMLPEYKEGEPYFEKMDYKFLPDPNKMLPKVFLSKKGIEIYKPSPKLLEQYGHGTHKKGDTFSMDDLHELIDFFKHSIEAHEDWKQFGFKFSDTATYENVSSFYREVEDQGYKLSFRKVSESYVYSLIDQGKLYLFQIYNKDFSPCSKGTPNLHTLYWRMLFDERNLADVIYKLDGKAEIFFREKSLKNDHPTHPAGKPIKKKSRQKKGEESLFEYDLVKDRRYTMDKFQFHVPITMNFKCSAGSKVNDMVNAHIREAKDMHVIGIDRGERNLLYICVIDSRGTILDQISLNTINDIDYHDLLESRDKDRQQEHRNWQTIEGIKELKQGYLSQAVHRIAELMVAYKAVVALEDLNMGFKRGRQKVESSVYQQFEKQLIDKLNYLVDKKKRPEDIGGLLRAYQFTAPFKSFKEMGKQNGFLFYIPAWNTSNIDPTTGFVNLFHVQYENVDKAKSFFQKFDSISYNPKKDWFEFAFDYKNFTKKAEGSRSMWILCTHGSRIKNFRNSQKNGQWDSEEFALTEAFKSLFVRYEIDYTADLKTAIVDEKQKDFFVDLLKLFKLTVQMRNSWKEKDLDYLISPVAGADGRFFDTREGNKSLPKDADANGAYNIALKGLWALRQIRQTSEGGKLKLAISNKEWLQFVQERSYEKD

SEQ ID NO: 764 - protein, 1241 aa, synthetic construct
MLLYENYTKRNQITKSLRLELRPQGKTLRNIKELNLLEQDKAIYALLERLKPVIDEGIKDIARDTLKNCELSFEKLYEHFLSGDKKAYAKESERLKKEIVKTLIKNLPEGIGKISEINSAKYLNGVLYDFIDKTHKDSEEKQNILSDILETKGYLALFSKFLTSRITTLEQSMPKRVIENFEIYAANIPKMQDALERGAVSFAIEYESICSVDYYNQILSQEDIDSYNRLISGIMDEDGAKEKGINQTISEKNIKIKSEHLEEKPFRILKQLHKQILEEREKAFTIDHIDSDEEVVQVTKEAFEQTKEQWENIKKINGFYAKDPGDITLFIVVGPNQTHVLSQLIYGEHDRIRLLLEEYEKNTLEVLPRRTKSEKARYDKFVNAVPKKVAKESHTFDGLQKMTGDDRLFILYRDELARNYMRIKEAYGTFERDILKSRRGIKGNRDVQESLVSFYDELTKFRSALRIINSGNDEKADPIFYNTFDGIFEKANRTYKAENLCRNYVTKSPADDARIMASCLGTPARLRTHWWNGEENFAINDVAMIRRGDEYYYFVLTPDVKPVDLKTKDETDAQIFVQRKGAKSFLGLPKALFKCILEPYFESPEHKNDKNCVIEEYVSKPLTIDRRAYDIFKNGTFKKTNIGIDGLTEEKFKDDCRYLIDVYKEFIAVYTRYSCFNMSGLKRADEYNDIGEFFSDVDTRLCTMEWIPVSFERINDMVDKKEGLLFLVRSMFLYNRPRKPYERTFIQLFSDSNMEHTSMLLNSRAMIQYRAASLPRRVTHKKGSILVALRDSNGEHIPMHIREAIYKMKNNFDISSEDFIMAKAYLAEHDVAIKKANEDIIRNRRYTEDKFFLSLSYTKNADISARTLDYINDKVEEDTQDSRMAVIVTRNLKDLTYVAVVDEKNNVLEEKSLNEIDGVNYRELLKERTKIKYHDKTRLWQYDVSSKGLKEAYVELAVTQISKLATKYNAVVVVESMSSTFKDKFSFLDEQIFKAFEARLCARMSDLSFNTIKEGEAGSISNPIQVSNNNGNSYQDGVIYFLNNAYTRTLCPDTGFVDVFDKTRLITMQSKRQFFAKMKDIRIDDGEMLFTFNLEEYPTKRLLDRKEWTVKIAGDGSYFDKDKGEYVYVNDIVREQIIPALLEDKAVFDGNMAEKFLDKTAISGKSVELIYKWFANALYGIITKKDGEKIYRSPITGTEIDVSKNTTYNFGKKFMFKQEYRGDGDFLDAFLNYMQAQDIAV

SEQ ID NO: 765 - protein, 1263 aa, synthetic construct
MEDYSGFVNIYSIQKTLRFELKPVGKTLEHIEKKGFLKKDKIRAEDYKAVKKIIDKYHRAYIEEVFDSVLHQKKKKDKTRFSTQFIKEIKEFSELYYKTEKNIPDKERLEALSEKLRKMLVGAFKGEFSEEVAEKYKNLFSKELIRNEIEKFCETDEERKQVSNFKSFTTYFTGFHSNRQNIYSDEKKSTAIGYRIIHQNLPKFLDNLKIIESIQRRFKDFPWSDLKKNLKKIDKNIKLTEYFSIDGFVNVLNQKGIDAYNTILGGKSEESGEKIQGLNEYINLYRQKNNIDRKNLPNVKILFKQILGDRETKSFIPEAFPDDQSVLNSITEFAKYLKLDKKKKSIIAELKKFLSSFNRYELDGIYLANDNSLASISTFLFDDWSFIKKSVSFKYDESVGDPKKKIKSPLKYEKEKEKWLKQKYYTISFLNDAIESYSKSQDEKRVKIRLEAYFAEFKSKDDAKKQFDLLERIEEAYAIVEPLLGAEYPRDRNLKADKKEVGKIKDFLDSIKSLQFFLKPLLSAEIFDEKDLGFYNQLEGYYEEIDSIGHLYNKVRNYLTGKIYSKEKFKLNFENSTLLKGWDENREVANLCVIFREDQKYYLGVMDKENNTILSDIPKVKPNELFYEKMVYKLIPTPHMQLPRIIFSSDNLSIYNPSKSILKIREAKSFKEGKNFKLKDCHKFIDFYKESISKNEDWSRFDFKFSKTSSYENISEFYREVERQGYNLDFKKVSKFYIDSLVEDGKLYLFQIYNKDFSIFSKGKPNLHTIYFRSLFSKENLKDVCLKLNGEAEMFFRKKSINYDEKKKREGHHPELFEKLKYPILKDKRYSEDKFQFHLPISLNFKSKERLNFNLKVNEFLKRNKDINIIGIDRGERNLLYLVMINQKGEILKQTLLDSMQSGKGRPEINYKEKLQEKEIERDKARKSWGTVENIKELKEGYLSIVIHQISKLMVENNAIVVLEDLNIGFKRGRQKVERQVYQKFEKMLIDKLNFLVFKENKPTEPGGVLKAYQLTDEFQSFEKLSKQTGFLFYVPSWNTSKIDPRTGFIDFLHPAYENIEKAKQWINKFDSIRFNSKMDWFEFTADTRKFSENLMLGKNRVWVICTTNVERYFTSKTANSSIQYNSIQITEKLKELFVDIPFSNGQDLKPEILRKNDAVFFKSLLFYIKTTLSLRQNNGKKGEEEKDFILSPVVDSKGRFFNSLEASDDEPKDADANGAYHIALKGLMNLLVLNETKEENLSRPKWKIKNKDWLEFVWERNR

SEQ ID NO: 766 - protein, 1246 aa, synthetic construct
MKTQHFFEDFTSLYSLSKTIRFELKPIGKTLENIKKNGLIRRDEQRLDDYEKLKKVIDEYHEDFIANILSSFSFSEEILQSYIQNLSESEARAKIEKTMRDTLAKAFSEDERYKSIFKKELVKKDIPVWCPAYKSLCKKFDNFTTSLVPFHENRKNLYTSNEITASIPYRIVHVNLPKFIQNIEALCELQKKMGADLYLEMMENLRNVWPSFVKTPDDLCNLKTYNHLMVQSSISEYNRFVGGYSTEDGTKHQGINEWINIYRQRNKEMRLPGLVFLHKQILAKVDSSSFISDTLENDDQVFCVLRQFRKLFWNTVSSKEDDAASLKDLFCGLSGYDPEAIYVSDAHLATISKNIFDRWNYISDAIRRKTEVLMPRKKESVERYAEKISKQIKKRQSYSLAELDDLLAHYSEESLPAGFSLLSYFTSLGGQKYLVSDGEVILYEEGSNIWDEVLIAFRDLQVILDKDFTEKKLGKDEEAVSVIKKALDSALRLRKFFDLLSGTGAEIRRDSSFYALYTDRMDKLKGLLKMYDKVRNYLTKKPYSIEKFKLHFDNPSLLSGWDKNKELNNLSVIFRQNGYYYLGIMTPKGKNLFKTLPKLGAEEMFYEKMEYKQIAEPMLMLPKVFFPKKTKPAFAPDQSVVDIYNKKTFKTGQKGFNKKDLYRLIDFYKEALTVHEWKLFNFSFSPTEQYRNIGEFFDEVREQAYKVSMVNVPASYIDEAVENGKLYLFQIYNKDFSPYSKGIPNLHTLYWKALFSEQNQSRVYKLCGGGELFYRKASLHMQDTTVHPKGISIHKKNLNKKGETSLFNYDLVKDKRFTEDKFFFHVPISINYKNKKITNVNQMVRDYIAQNDDLQIIGIDRGERNLLYISRIDTRGNLLEQFSLNVIESDKGDLRTDYQKILGDREQERLRRRQEWKSIESIKDLKDGYMSQVVHKICNMVVEHKAIVVLENLNLSFMKGRKKVEKSVYEKFERMLVDKLNYLVVDKKNLSNEPGGLYAAYQLTNPLFSFEELHRYPQSGILFFVDPWNTSLTDPSTGFVNLLGRINYTNVGDARKFFDRFNAIRYDGKGNILFDLDLSRFDVRVETQRKLWTLTTFGSRIAKSKKSGKWMVERIENLSLCFLELFEQFNIGYRVEKDLKKAILSQDRKEFYVRLIYLFNLMMQIRNSDGEEDYILSPALNEKNLQFDSRLIEAKDLPVDADANGAYNVARKGLMVVQRIKRGDHESIHRIGRAQWLRYVQEGIVE

SEQ ID NO: 767 - protein, 1206 aa, synthetic construct
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 768 - protein, 1352 aa, synthetic construct
MENIFDQFIGKYSLSKTLRFELKPVGKTEDFLKINKVFEKDQTIDDSYNQAKFYFDSLHQKFIDAALASDKTSELSFQNFADVLEKQNKIILDKKREMGALRKRDKNAVGIDRLQKEINDAEDIIQKEKEKIYKDVRTLFDNEAESWKTYYQEREVDGKKITFSKADLKQKGADFLTAAGILKVLKYEFPEEKEKEFQAKNQPSLFVEEKENPGQKRYIFDSFDKFAGYLTKFQQTKKNLYAADGTSTAVATRIADNFIIFHQNTKVFRDKYKNNHTDLGFDEENIFEIERYKNCLLQREIEHIKNENSYNKIIGRINKKIKEYRDQKAKDTKLTKSDFPFFKNLDKQILGEVEKEKQLIEKTREKTEEDVLIERFKEFIENNEERFTAAKKLMNAFCNGEFESEYEGIYLKNKAINTISRRWFVSDRDFELKLPQQKSKNKSEKNEPKVKKFISIAEIKNAVEELDGDIFKAVFYDKKIIAQGGSKLEQFLVIWKYEFEYLFRDIERENGEKLLGYDSCLKIAKQLGIFPQEKEAREKATAVIKNYADAGLGIFQMMKYFSLDDKDRKNTPGQLSTNFYAEYDGYYKDFEFIKYYNEFRNFITKKPFDEDKIKLNFENGALLKGWDENKEYDFMGVILKKEGRLYLGIMHKNHRKLFQSMGNAKGDNANRYQKMIYKQIADASKDVPRLLLTSKKAMEKFKPSQEILRIKKEKTFKRESKNFSLRDLHALIEYYRNCIPQYSNWSFYDFQFQDTGKYQNIKEFTDDVQKYGYKISFRDIDDEYINQALNEGKMYLFEVVNKDIYNTKNGSKNLHTLYFEHILSAENLNDPVFKLSGMAEIFQRQPSVNEREKITTQKNQCILDKGDRAYKYRRYTEKKIMFHMSLVLNTGKGEIKQVQFNKIINQRISSSDNEMRVNVIGIDRGEKNLLYYSVVKQNGEIIEQASLNEINGVNYRDKLIEREKERLKNRQSWKPVVKIKDLKKGYISHVIHKICQLIEKYSAIVVLEDLNMRFKQIRGGIERSVYQQFEKALIDKLGYLVFKDNRDLRAPGGVLNGYQLSAPFVSFEKMRKQTGILFYTQAEYTSKTDPITGFRKNVYISNSASLDKIKEAVKKFDAIGWDGKEQSYFFKYNPYNLADEKYKNSTVSKEWAIFASAPRIRRQKGEDGYWKYDRVKVNEEFEKLLKVWNFVNPKATDIKQEIIKKEKAGDLQGEKELDGRLRNFWHSFIYLFNLVLELRNSFSLQIKIKAGEVIAVDEGVDFIASPVKPFFTTPNPYIPSNLCWLAVENADANGAYNIARKGVMILKKIREHAKKDPEFKKLPNLFISNAEWDEAARDWGKYAGTTALNLDH

SEQ ID NO: 769 - protein, 1477 aa, synthetic construct
MSNFFKNFTNLYELSKTLRFELKPVGDTLTNMKDHLEYDEKLQTFLKDQNIDDAYQALKPQFDEIHEEFITDSLESKKAKEIDFSEYLDLFQEKKELNDSEKKLRNKIGETFNKAGEKWKKEKYPQYEWKKGSKIANGADILSCQDMLQFIKYKNPEDEKIKNYIDDTLKGFFTYFGGFNQNRANYYETKKEASTAVATRIVHENLPKFCDNVIQFKHIIKRKKDGTVEKTERKTEYLNAYQYLKNNNKITQIKDAETEKMIESTPIAEKIFDVYYFSSCLSQKQIEEYNRIIGHYNLLINLYNQAKRSEGKHLSANEKKYKDLPKFKTLYKQIGCGKKKDLFYTIKCDTEEEANKSRNEGKESHSVEEIINKAQEAINKYFKSNNDCENINTVPDFINYILTKENYEGVYWSKAAMNTISDKYFANYHDLQDRLKEAKVFQKADKKSEDDIKIPEAIELSGLFGVLDSLADWQTTLFKSSILSNEDKLKIITDSQTPSEALLKMIFNDIEKNMESFLKETNDIITLKKYKGNKEGTEKIKQWFDYTLAINRMLKYFLVKENKIKGNSLDTNISEALKTLIYSDDAEWFKWYDALRNYLTQKPQDEAKENKLKLNFDNPSLAGGWDVNKECSNFCVILKDKNEKKYLAIMKKGENTLFQKEWTEGRGKNLTKKSNPLFEINNCEILSKMEYDFWADVSKMIPKCSTQLKAVVNHFKQSDNEFIFPIGYKVTSGEKFREECKISKQDFELNNKVFNKNELSVTAMRYDLSSTQEKQYIKAFQKEYWELLFKQEKRDTKLTNNEIFNEWINFCNKKYSELLSWERKYKDALTNWINFCKYFLSKYPKTTLFNYSFKESENYNSLDEFYRDVDICSYKLNINTTINKSILDRLVEEGKLYLFEIKNQDSNDGKSIGHKNNLHTIYWNAIFENFDNRPKLNGEAEIFYRKAISKDKLGIVKGKKTKNGTEIIKNYRFSKEKFILHVPITLNFCSNNEYVNDIVNTKFYNFSNLHFLGIDRGEKHLAYYSLVNKNGEIVDQGTLNLPFTDKDGNQRSIKKEKYFYNKQEDKWEAKEVDCWNYNDLLDAMASNRDMARKNWQRIGTIKEAKNGYVSLVIRKIADLAVNNERPAFIVLEDLNTGFKRSRQKIDKSVYQKFELALAKKLNFLVDKNAKRDEIGSPTKALQLTPPVNNYGDIENKKQAGIMLYTRANYTSQTDPATGWRKTIYLKAGPEETTYKKDGKIKNKSVKDQIIETFTDIGFDGKDYYFEYDKGEFVDEKTGEIKPKKWRLYSGENGKSLDRFRGEREKDKYEWKIDKIDIVKILDDLFVNFDKNISLLKQLKEGVELTRNNEHGTGESLRFAINLIQQIRNTGNNERDNDFILSPVRDENGKHFDSREYWDKETKGEKISMPSSGDANGAFNIARKGIIMNAHILANSDSKDLSLFVSDEEWDLHLNNKTEWKKQLNIFSSRKAMAKRKK

SEQ ID NO: 770 - protein, 1323 aa, synthetic construct
MENYQEFTNLFQLNKTLRFELKPIGKTCELLEEGKIFASGSFLEKDKVRADNVSYVKKEIDKKHKIFIEETLSSFSISNDLLKQYFDCYNELKAFKKDCKSDEEEVKKTALRNKCTSIQRAMREAISQAFLKSPQKKLLAIKNLIENVFKADENVQHFSEFTSYFSGFETNRENFYSDEEKSTSIAYRLVHDNLPIFIKNIYIFEKLKEQFDAKTLSEIFENYKLYVAGSSLDEVFSLEYFNNTLTQKGIDNYNAVIGKIVKEDKQEIQGLNEHINLYNQKHKDRRLPFFISLKKQILSDREALSWLPDMFKNDSEVIKALKGFYIEDGFENNVLTPLATLLSSLDKYNLNGIFIRNNEALSSLSQNVYRNFSIDEAIDANAELQTFNNYELIANALRAKIKKETKQGRKSFEKYEEYIDKKVKAIDSLSIQEINELVENYVSEFNSNSGNMPRKVEDYFSLMRKGDFGSNDLIENIKTKLSAAEKLLGTKYQETAKDIFKKDENSKLIKELLDATKQFQHFIKPLLGTGEEADRDLVFYGDFLPLYEKFEELTLLYNKVRNRLTQKPYSKDKIRLCFNKPKLMTGWVDSKTEKSDNGTQYGGYLFRKKNEIGEYDYFLGISSKAQLFRKNEAVIGDYERLDYYQPKANTIYGSAYEGENSYKEDKKRLNKVIIAYIEQIKQTNIKKSIIESISKYPNISDDDKVTPSSLLEKIKKVSIDSYNGILSFKSFQSVNKEVIDNLLKTISPLKNKAEFLDLINKDYQIFTEVQAVIDEICKQKTFIYFPISNVELEKEMGDKDKPLCLFQISNKDLSFAKTFSANLRKKRGAENLHTMLFKALMEGNQDNLDLGSGAIFYRAKSLDGNKPTHPANEAIKCRNVANKDKVSLFTYDIYKNRRYMENKFLFHLSIVQNYKAANDSAQLNSSATEYIRKADDLHIIGIDRGERNLLYYSVIDMKGNIVEQDSLNIIRNNDLETDYHDLLDKREKERKANRQNWEAVEGIKDLKKGYLSQAVHQIAQLMLKYNAIIALEDLGQMFVTRGQKIEKAVYQQFEKSLVDKLSYLVDKKRPYNELGGILKAYQLASSITKNNSDKQNGFLFYVPAWNTSKIDPVTGFTDLLRPKAMTIKEAQDFFGAFDNISYNDKGYFEFETNYDKFKIRMKSAQTRWTICTFGNRIKRKKDKNYWNYEEVELTEEFKKLFKDSNIDYENCNLKEEIQNKDNRKFFDDLIKLLQLTLQMRNSDDKGNDYIISPVANAEGQFFDSRNGDKKLPLDADANGAYNIARKGLWNIRQIKQTKNDKKLNLSISSTEWLDFVREKPYLK

SEQ ID NO: 771 - protein, 1373 aa, synthetic construct
MLFQDFTHLYPLSKTVRFELKPIDRTLEHIHAKNFLSQDETMADMHQKVKVILDDYHRDFIADMMGEVKLTKLAEFYDVYLKFRKNPKDDELQKQLKDLQAVLRKEIVKPIGNGGKYKAGYDRLFGAKLFKDGKELGDLAKFVIAQEGESSPKLAHLAHFEKFSTYFTGFHDNRKNMYSDEDKHTAIAYRLIHENLPRFIDNLQILTTIKQKHSALYDQIINELTASGLDVSLASHLDGYHKLLTQEGITAYNTLLGGISGEAGSPKIQGINELINSHHNQHCHKSERIAKLRPLHKQILSDGMSVSFLPSKFADDSEMCQAVNEFYRHYADVFAKVQSLFDGFDDHQKDGIYVEHKNLNELSKQAFGDFALLGRVLDGYYVDVVNPEFNERFAKAKTDNAKAKLTKEKDKFIKGVHSLASLEQAIEHYTARHDDESVQAGKLGQYFKHGLAGVDNPIQKIHNNHSTIKGFLERERPAGERALPKIKSGKNPEMTQLRQLKELLDNALNVAHFAKLLTTKTTLDNQDGNFYGEFGVLYDELAKIPTLYNKVRDYLSQKPFSTEKYKLNFGNPTLLNGWDLNKEKDNFGVILQKDGCYYLALLDKAHKKVFDNAPNTGKSIYQKMIYKYLEVRKQFPKVFFSKEAIAINYHPSKELVEIKDKGRQRSDDERLKLYRFILECLKIHPKYDKKFEGAIGDIQLFKKDKKGREVPISEKDLFDKINGIFSSKPKLEMEDFFIGEFKRYNPSQDLVDQYNIYKKIDSNDNRKKENFYNNHPKFKKDLVRYYYESMCKHEEWEESFEFSKKLQDIGCYVDVNELFTEIETRRLNYKISFCNINADYIDELVEQGQLYLFQIYNKDFSPKAHGKPNLHTLYFKALFSEDNLADPIYKLNGEAQIFYRKASLDMNETTIHRAGEVLENKNPDNPKKRQFVYDIIKDKRYTQDKFMLHVPITMNFGVQGMTIKEFNKKVNQSIQQYDEVNVIGIDRGERHLLYLTVINSKGEILEQCSLNDITTASANGTQMTTPYHKILDKREIERLNARVGWGEIETIKELKSGYLSHVVHQISQLMLKYNAIVVLEDLNFGFKRGRFKVEKQIYQNFENALIKKLNHLVLKDKADDEIGSYKNALQLTNNFTDLKSIGKQTGFLFYVPAWNTSKIDPETGFVDLLKPRYENIAQSQAFFGKFDKICYNADKDYFEFHIDYAKFTDKAKNSRQIWTICSHGDKRYVYDKTANQNKGAAKGINVNDELKSLFARHHINEKQPNLVMDICQNNDKEFHKSLMYLLKTLLALRYSNASSDEDFILSPVANDEGVFFNSALADDTQPQNADANGAYHIALKGLWLLNELKNSDDLNKVKLAIDNQTWLNFAQNR

SEQ ID NO: 772 - protein, 1282 aa, synthetic construct
MNGNRSIVYREFVGVIPVAKTLRNELRPVGHTQEHIIQNGLIQEDELRQEKSTELKNIMDDYYREYIDKSLSGVTDLDFTLLFELMNLVQSSPSKDNKKALEKEQSKMREQICTHLQSDSNYKNIFNAKLLKEILPDFIKNYNQYDVKDKAGKLETLALFNGFSTYFTDFFEKRKNVFTKEAVSTSIAYRIVHENSLIFLANMTSYKKISEKALDEIEVIEKNNQDKMGDWELNQIFNPDFYNMVLIQSGIDFYNEICGVVNAHMNLYCQQTKNNYNLFKMRKLHKQILAYTSTSFEVPKMFEDDMSVYNAVNAFIDETEKGNIIGKLKDIVNKYDELDEKRIYISKDFYETLSCFMSGNWNLITGCVENFYDENIHAKGKSKEEKVKKAVKEDKYKSINDVNDLVEKYIDEKERNEFKNSNAKQYIREISNIITDTETAHLEYDDHISLIESEEKADEMKKRLDMYMNMYHWAKAFIVDEVLDRDEMFYSDIDDIYNILENIVPLYNRVRNYVTQKPYNSKKIKLNFQSPTLANGWSQSKEFDNNAIILIRDNKYYLAIFNAKNKPDKKIIQGNSDKKNDNDYKKMVYNLLPGANKMLPKVFLSKKGIETFKPSDYIISGYNAHKHIKTSENFDISFCRDLIDYFKNSIEKHAEWRKYEFKFSATDSYSDISEFYREVEMQGYRIDWTYISEADINKLDEEGKIYLFQIYNKDFAENSTGKENLHTMYFKNIFSEENLKDIIIKLNGQAELFYRRASVKNPVKHKKDSVLVNKTYKNQLDNGDVVRIPIPDDIYNEIYKMYNGYIKESDLSEAAKEYLDKVEVRTAQKDIVKDYRYTVDKYFIHTPITINYKVTARNNVNDMVVKYIAQNDDIHVIGIDRGERNLIYISVIDSHGNIVKQKSYNILNNYDYKKKLVEKEKTREYARKNWKSIGNIKELKEGYISGVVHEIAMLIVEYNAIIAMEDLNYGFKRGRFKVERQVYQKFESMLINKLNYFASKEKSVDEPGGLLKGYQLTYVPDNIKNLGKQCGVIFYVPAAFTSKIDPSTGFISAFNFKSISTNASRKQFFMQFDEIRYCAEKDMFSFGFDYNNFDTYNITMGKTQWTVYTNGERLQSEFNNARRTGKTKSINLTETIKLLLEDNEINYADGHDIRIDMEKMDEDKKSEFFAQLLSLYKLTVQMRNSYTEAEEQENGISYDKIISPVINDEGEFFDSDNYKESDDKECKMPKDADANGAYCIALKGLYEVLKIKSEWTEDGFDRNCLKLPHAEWLDFIQNKRYE

SEQ ID NO: 773 - protein, 1238 aa, synthetic construct
MNNYDEFTKLYPIQKTIRFELKPQGRTMEHLETFNFFEEDRDRAEKYKILKEAIDEYHKKFIDEHLTNMSLDWNSLKQISEKYYKSREEKDKKVFLSEQKRMRQEIVSEFKKDDRFKDLFSKKLFSELLKEEIYKKGNHQEIDALKSFDKFSGYFIGLHENRKNMYSDGDEITAISNRIVNENFPKFLDNLQKYQEARKKYPEWIIKAESALVAHNIKMDEVFSLEYFNKVLNQEGIQRYNLALGGYVTKSGEKMMGLNDALNLAHQSEKSSKGRIHMTPLFKQILSEKESFSYIPDVFTEDSQLLPSIGGFFAQIENDKDGNIFDRALELISSYAEYDTERIYIRQADINRVSNVIFGEWGTLGGLMREYKADSINDINLERTCKKVDKWLDSKEFALSDVLEAIKRTGNNDAFNEYISKMRTAREKIDAARKEMKFISEKISGDEESIHIIKTLLDSVQQFLHFFNLFKARQDIPLDGAFYAEFDEVHSKLFAIVPLYNKVRNYLTKNNLNTKKIKLNFKNPTLANGWDQNKVYDYASLIFLRDGNYYLGIINPKRKKNIKFEQGSGNGPFYRKMVYKQIPGPNKNLPRVFLTSTKGKKEYKPSKEIIEGYEADKHIRGDKFDLDFCHKLIDFFKESIEKHKDWSKFNFYFSPTESYGDISEFYLDVEKQGYRMHFENISAETIDEYVEKGDLFLFQIYNKDFVKAATGKKDMHTIYWNAAFSPENLQDVVVKLNGEAELFYRDKSDIKEIVHREGEILVNRTYNGRTPVPDKIHKKLTDYHNGRTKDLGEAKEYLDKVRYFKAHYDITKDRRYLNDKIYFHVPLTLNFKANGKKNLNKMVIEKFLSDEKAHIIGIDRGERNLLYYSIIDRSGKIIDQQSLNVIDGFDYREKLNQREIEMKDARQSWNAIGKIKDLKEGYLSKAVHEITKMAIQYNAIVVMEELNYGFKRGRFKVEKQIYQKFENMLIDKMNYLVFKDAPDESPGGVLNAYQLTNPLESFAKLGKQTGILFYVPAAYTSKIDPTTGFVNLFNTSSKTNAQERKEFLQKFESISYSAKDGGIFAFAFDYRKFGTSKTDHKNVWTAYTNGERMRYIKEKKRNELFDPSKEIKEALTSSGIKYDGGQNILPDILRSNNNGLIYTMYSSFIAAIQMRVYDGKEDYIISPIKNSKGEFFRTDPKRRELPIDADANGAYNIALRGELTMRAIAEKFDPDSEKMAKLELKHKDWFEFMQTRGD

SEQ ID NO: 774 - protein, 1206 aa, synthetic construct
MYYQNLTKKYPVSKTIRNELIPIGKTLENIRKNNILESDVKRKQDYEHVKGIMDEYHKQLINEALDNYMLPSLNQAAEIYLKKHVDVEDREEFKKTQDLLRREVTGRLKEHENYTKIGKKDILDLLEKLPSISEEDYNALESFRNFYTYFTSYNKVRENLYSDEEKSSTVAYRLINENLPKFLDNIKSYAFVKAAGVLADCIEEEEQDALFMVETFNMTLTQEGIDMYNYQIGKVNSAINLYNQKNHKVEEFKKIPKMKVLYKQILSDREEVFIGEFKDDETLLSSIGAYGNVLMTYLKSEKINIFFDALRESEGKNVYVKNDLSKTTMSNIVFGSWSAFDELLNQEYDLANENKKKDDKYFEKRQKELKKNKSYTLEQMSNLSKEDISPIENYIERISEDIEKICIYNGEFEKIVVNEHDSSRKLSKNIKAVKVIKDYLDSIKELEHDIKLINGSGQELEKNLVVYVGQEEALEQLRPVDSLYNLTRNYLTKKPFSTEKVKLNFNKSTLLNGWDKNKETDNLGILFFKDGKYYLGIMNTTANKAFVNPPAAKTENVFKKVDYKLLPGSNKMLPKVFFAKSNIGYYNPSTELYSNYKKGTHKKGPSFSIDDCHNLIDFFKESIKKHEDWSKFGFEFSDTADYRDISEFYREVEKQGYKLTFTDIDESYINDLIEKNELYLFQIYNKDFSEYSKGKLNLHTLYFMMLFDQRNLDNVVYKLNGEAEVFYRPASIAENELVIHKAGEGIKNKNPNRAKVKETSTFSYDIVKDKRYSKYKFTLHIPITMNFGVDEVRRFNDVINNALRTDDNVNVIGIDRGERNLLYVVVINSEGKILEQISLNSIINKEYDIETNYHALLDEREDDRNKARKDWNTIENIKELKTGYLSQVVNVVAKLVLKYNAIICLEDLNFGFKRGRQKVEKQVYQKFEKMLIEKLNYLVIDKSREQVSPEKMGGALNALQLTSKFKSFAELGKQSGIIYYVPAYLTSKIDPTTGFVNLFYIKYENIEKAKQFFDGFDFIRFNKKDDMFEFSFDYKSFTQKACGIRSKWIVYTNGERIIKYPNPEKNNLFDEKVINVTDEIKGLFKQYRIPYENGEDIKEIIISKAEADFYKRLFRLLHQTLQMRNSTSDGTRDYIISPVKNDRGEFFCSEFSEGTMPKDADANGAYNIARKGLWVLEQIRQKDEGEKVNLSMTNAEWLKYAQLHLL

SEQ ID NO: 775 - protein, 1115 aa, synthetic construct
MGIHGVPAAATRSFILKIEPNEEVKKGLWKTHEVLNHGIAYYMNILKLIRQEAIYEHHEQDPKNPKKVSKAEIQAELWDFVLKMQKCNSFTHEVDKDEVFNILRELYEELVPSSVEKKGEANQLSNKFLYPLVDPNSQSGKGTASSGRKPRWYNLKIAGDPSWEEEKKKWEEDKKKDPLAKILGKLAEYGLIPLFIPYTDSNEPIVKEIKWMEKSRNQSVRRLDKDMFIQALERFLSWESWNLKVKEEYEKVEKEYKTLEERIKEDIQALKALEQYEKERQEQLLRDTLNTNEYRLSKRGLRGWREIIQKWLKMDENEPSEKYLEVFKDYQRKHPREAGDYSVYEFLSKKENHFIWRNHPEYPYLYATFCEIDKKKKDAKQQATFTLADPINHPLWVRFEERSGSNLNKYRILTEQLHTEKLKKKLTVQLDRLIYPTESGGWEEKGKVDIVLLPSRQFYNQIFLDIEEKGKHAFTYKDESIKFPLKGTLGGARVQFDRDHLRRYPHKVESGNVGRIYFNMTVNIEPTESPVSKSLKIHRDDFPKVVNFKPKELTEWIKDSKGKKLKSGIESLEIGLRVMSIDLGQRQAAAASIFEVVDQKPDIEGKLFFPIKGTELYAVHRASFNIKLPGETLVKSREVLRKAREDNLKLMNQKLNFLRNVLHFQQFEDITEREKRVTKWISRQENSDVPLVYQDELIQIRELMYKPYKDWVAFLKQLHKRLEVEIGKEVKHWRKSLSDGRKGLYGISLKNIDEIDRTRKFLLRWSLRPTEPGEVRRLEPGQRFAIDQLNHLNALKEDRLKKMANTIIMHALGYCYDVRKKKWQAKNPACQIILFEDLSNYNPYGERSRFENSRLMKWSRREIPRQVALQGEIYGLQVGEVGAQFSSRFHAKTGSPGIRCRVVTKEKLQDNRFFKNLQREGRLTLDKIAVLKEGDLYPDKGGEKFISLSKDRKCVTTHADINAAQNLQKRFWTRTHGFYKVYCKAYQVDGQTVYIPESKDQKQKIIEEFGEGYFILKDGVYEWVNAGKLKIKKGSSKQSSSELVDSDILKDSFDLASELKGEKLMLYRDPSGNVFPSDKWMAAGVFFGKLERILISKLTNQYSISTIEDDSSKQS

SEQ ID NO: 776 - protein, 1147 aa, synthetic construct
MAVKSIKVKLRLSECPDILAGMWQLHRATNAGVRYYTEWVSLMRQEILYSRGPDGGQQCYMTAEDCQRELLRRLRNRQLHNGRQDQPGTDADLLAISRRLYEILVLQSIGKRGDAQQIASSFLSPLVDPNSKGGRGEAKSGRKPAWQKMRDQGDPRWVAAREKYEQRKAVDPSKEILNSLDALGLRPLFAVFTETYRSGVDWKPLGKSQGVRTWDRDMFQQALERLMSWESWNRRVGEEYARLFQQKMKFEQEHFAEQSHLVKLARALEADMRAASQGFEAKRGTAHQITRRALRGADRVFEIWKSIPEEALFSQYDEVIRQVQAEKRRDFGSHDLFAKLAEPKYQPLWRADETFLTRYALYNGVLRDLEKARQFATFTLPDACVNPIWTRFESSQGSNLHKYEFLFDHLGPGRHAVRFQRLLVVESEGAKERDSVVVPVAPSGQLDKLVLREEEKSSVALHLHDTARPDGFMAEWAGAKLQYERSTLARKARRDKQGMRSWRRQPSMLMSAAQMLEDAKQAGDVYLNISVRVKSPSEVRGQRRPPYAALFRIDDKQRRVTVNYNKLSAYLEEHPDKQIPGAPGLLSGLRVMSVDLGLRTSASISVFRVAKKEEVEALGDGRPPHYYPIHGTDDLVAVHERSHLIQMPGETETKQLRKLREERQAVLRPLFAQLALLRLLVRCGAADERIRTRSWQRLTKQGREFTKRLTPSWREALELELTRLEAYCGRVPDDEWSRIVDRTVIALWRRMGKQVRDWRKQVKSGAKVKVKGYQLDVVGGNSLAQIDYLEQQYKFLRRWSFFARASGLVVRADRESHFAVALRQHIENAKRDRLKKLADRILMEALGYVYEASGPREGQWTAQHPPCQLIILEELSAYRFSDDRPPSENSKLMAWGHRGILEELVNQAQVHDVLVGTVYAAFSSRFDARTGAPGVRCRRVPARFVGATVDDSLPLWLTEFLDKHRLDKNLLRPDDVIPTGEGEFLVSPCGEEAARVRQVHADINAAQNLQRRLWQNFDITELRLRCDVKMGGEGTVLVPRVNNARAKQLFGKKVLVSQDGVTFFERSQTGGKPHSEKQTDLTDKELELIAEADEARAKSVVLFRDPSGHIGKGHWIRQREFWSLVKQRIESHTAERIRVRGVGSSLD

SEQ ID NO: 777 - protein, 1108 aa, synthetic construct
MAIRSIKLKLKTHTGPEAQNLRKGIWRTHRLLNEGVAYYMKMLLLFRQESTGERPKEELQEELICHIREQQQRNQADKNTQALPLDKALEALRQLYELLVPSSVGQSGDAQIISRKFLSPLVDPNSEGGKGTSKAGAKPTWQKKKEANDPTWEQDYEKWKKRREEDPTASVITTLEEYGIRPIFPLYTNTVTDIAWLPLQSNQFVRTWDRDMLQQAIERLLSWESWNKRVQEEYAKLKEKMAQLNEQLEGGQEWISLLEQYEENRERELRENMTAANDKYRITKRQMKGWNELYELWSTFPASASHEQYKEALKRVQQRLRGRFGDAHFFQYLMEEKNRLIWKGNPQRIHYFVARNELTKRLEEAKQSATMTLPNARKHPLWVRFDARGGNLQDYYLTAEADKPRSRRFVTFSQLIWPSESGWMEKKDVEVELALSRQFYQQVKLLKNDKGKQKIEFKDKGSGSTFNGHLGGAKLQLERGDLEKEEKNFEDGEIGSVYLNVVIDFEPLQEVKNGRVQAPYGQVLQLIRRPNEFPKVTTYKSEQLVEWIKASPQHSAGVESLASGFRVMSIDLGLRAAAATSIFSVEESSDKNAADFSYWIEGTPLVAVHQRSYMLRLPGEQVEKQVMEKRDERFQLHQRVKFQIRVLAQIMRMANKQYGDRWDELDSLKQAVEQKKSPLDQTDRTFWEGIVCDLTKVLPRNEADWEQAVVQIHRKAEEYVGKAVQAWRKRFAADERKGIAGLSMWNIEELEGLRKLLISWSRRTRNPQEVNRFERGHTSHQRLLTHIQNVKEDRLKQLSHAIVMTALGYVYDERKQEWCAEYPACQVILFENLSQYRSNLDRSTKENSTLMKWAHRSIPKYVHMQAEPYGIQIGDVRAEYSSRFYAKTGTPGIRCKKVRGQDLQGRRFENLQKRLVNEQFLTEEQVKQLRPGDIVPDDSGELFMTLTDGSGSKEVVFLQADINAAHNLQKRFWQRYNELFKVSCRVIVRDEEEYLVPKTKSVQAKLGKGLFVKKSDTAWKDVYVWDSQAKLKGKTTFTEESESPEQLEDFQEIIEEAEEAKGTYRTLFRDPSGVFFPESVWYPQKDFWGEVKRKLYGKLRERFLTKAR

SEQ ID NO: 778 - protein, 1146 aa, synthetic construct
MNVAVKSIKVKLMLGHLPEIREGLWHLHEAVNLGVRYYTEWLALLRQGNLYRRGKDGAQECYMTAEQCRQELLVRLRDRQKRNGHTGDPGTDEELLGVARRLYELLVPQSVGKKGQAQMLASGFLSPLADPKSEGGKGTSKSGRKPAWMGMKEAGDSRWVEAKARYEANKAKDPTKQVIASLEMYGLRPLFDVFTETYKTIRWMPLGKHQGVRAWDRDMFQQSLERLMSWESWNERVGAEFARLVDRRDRFREKHFTGQEHLVALAQRLEQEMKEASPGFESKSSQAHRITKRALRGADGIIDDWLKLSEGEPVDRFDEILRKRQAQNPRRFGSHDLFLKLAEPVFQPLWREDPSFLSRWASYNEVLNKLEDAKQFATFTLPSPCSNPVWARFENAEGTNIFKYDFLFDHFGKGRHGVRFQRMIVMRDGVPTEVEGIVVPIAPSRQLDALAPNDAASPIDVFVGDPAAPGAFRGQFGGAKIQYRRSALVRKGRREEKAYLCGFRLPSQRRTGTPADDAGEVFLNLSLRVESQSEQAGRRNPPYAAVFHISDQTRRVIVRYGEIERYLAEHPDTGIPGSRGLTSGLRVMSVDLGLRTSAAISVFRVAHRDELTPDAHGRQPFFFPIHGMDHLVALHERSHLIRLPGETESKKVRSIREQRLDRLNRLRSQMASLRLLVRTGVLDEQKRDRNWERLQSSMERGGERMPSDWWDLFQAQVRYLAQHRDASGEAWGRMVQAAVRTLWRQLAKQVRDWRKEVRRNADKVKIRGIARDVPGGHSLAQLDYLERQYRFLRSWSAFSVQAGQVVRAERDSRFAVALREHIDNGKKDRLKKLADRILMEALGYVYVTDGRRAGQWQAVYPPCQLVLLEELSEYRFSNDRPPSENSQLMVWSHRGVLEELIHQAQVHDVLVGTIPAAFSSRFDARTGAPGIRCRRVPSIPLKDAPSIPIWLSHYLKQTERDAAALRPGELIPTGDGEFLVTPAGRGASGVRVVHADINAAHNLQRRLWENFDLSDIRVRCDRREGKDGTVVLIPRLTNQRVKERYSGVIFTSEDGVSFTVGDAKTRRRSSASQGEGDDLSDEEQELLAEADDARERSVVLFRDPSGFVNGGRWTAQRAFWGMVHNRIETLLAERFSVSGAAEKVRG

SEQ ID NO: 779 - protein, 1129 aa, synthetic construct
MAVKSMKVKLRLDNMPEIRAGLWKLHTEVNAGVRYYTEWLSLLRQENLYRRSPNGDGEQECYKTAEECKAELLERLRARQVENGHCGPAGSDDELLQLARQLYELLVPQAIGAKGDAQQIARKFLSPLADKDAVGGLGIAKAGNKPRWVRMREAGEPGWEEEKAKAEARKSTDRTADVLRALADFGLKPLMRVYTDSDMSSVQWKPLRKGQAVRTWDRDMFQQAIERMMSWESWNQRVGEAYAKLVEQKSRFEQKNFVGQEHLVQLVNQLQQDMKEASHGLESKEQTAHYLTGRALRGSDKVFEKWEKLDPDAPFDLYDTEIKNVQRRNTRRFGSHDLFAKLAEPKYQALWREDASFLTRYAVYNSIVRKLNHAKMFATFTLPDATAHPIWTRFDKLGGNLHQYTFLFNEFGEGRHAIRFQKLLTVEDGVAKEVDDVTVPISMSAQLDDLLPRDPHELVALYFQDYGAEQHLAGEFGGAKIQYRRDQLNHLHARRGARDVYLNLSVRVQSQSEARGERRPPYAAVFRLVGDNHRAFVHFDKLSDYLAEHPDDGKLGSEGLLSGLRVMSVDLGLRTSASISVFRVARKDELKPNSEGRVPFCFPIEGNENLVAVHERSQLLKLPGETESKDLRAIREERQRTLRQLRTQLAYLRLLVRCGSEDVGRRERSWAKLIEQPMDANQMTPDWREAFEDELQKLKSLYGICGDREWTEAVYESVRRVWRHMGKQVRDWRKDVRSGERPKIRGYQKDVVGGNSIEQIEYLERQYKFLKSWSFFGKVSGQVIRAEKGSRFAITLREHIDHAKEDRLKKLADRIIMEALGYVYALDDERGKGKWVAKYPPCQLILLEELSEYQFNNDRPPSENNQLMQWSHRGVFQELLNQAQVHDLLVGTMYAAFSSRFDARTGAPGIRCRRVPARCAREQNPEPFPWWLNKFVAEHKLDGCPLRADDLIPTGEGEFFVSPFSAEEGDFHQIHADLNAAQNLQRRLWSDFDISQIRLRCDWGEVDGEPVLIPRTTGKRTADSYGNKVFYTKTGVTYYERERGKKRRKVFAQEELSEEEAELLVEADEAREKSVVLMRDPSGIINRGDWTRQKEFWSMVNQRIEGYLVKQIRSRVRLQESACENTGDI

SEQ ID NO: 780 - protein, 966 aa, synthetic construct
MIEKKKSFAKGMGVKSTLVSGSKVYMTTFAEGSDARLEKIVEGDSIRSVNEGEAFSAEMADKNAGYKIGNAKFSHPKGYAVVANNPLYTGPVQQDMLGLKETLEKRYFGESADGNDNICIQVIHNILDIEKILAEYITNAAYAVNNISGLDKDIIGFGKFSTVYTYDEFKDPEHHRAAFNNNDKLINAIKAQYDEFDNFLDNPRLGYFGQAFFSKEGRNYIINYGNECYDILALLSGLRHWVVHNNEEESRISRTWLYNLDKNLDNEYISTLNYLYDRITNELTNSFSKNSAANVNYIAETLGINPAEFAEQYFRFSIMKEQKNLGFNITKLREVMLDRKDMSEIRKNHKVFDSIRTKVYTMMDFVIYRYYIEEDAKVAAANKSLPDNEKSLSEKDIFVINLRGSFNDDQKDALYYDEANRIWRKLENIMHNIKEFRGNKTREYKKKDAPRLPRILPAGRDVSAFSKLMYALTMFLDGKEINDLLTTLINKFDNIQSFLKVMPLIGVNAKFVEEYAFFKDSAKIADELRLIKSFARMGEPIADARRAMYIDAIRILGTNLSYDELKALADTFSLDENGNKLKKGKHGMRNFIINNVISNKRFHYLIRYGDPAHLHEIAKNEAVVKFVLGRIADIQKKQGQNGKNQIDRYYETCIGKDKGKSVSEKVDALTKIITGMNYDQFDKKRSVIEDTGRENAEREKFKKIISLYLTVIYHILKNIVNINARYVIGFHCVERDAQLYKEKGYDINLKKLEEKGFSSVTKLCAGIDETAPDKRKDVEKEMAERAKESIDSLESANPKLYANYIKYSDEKKAEEFTRQINREKAKTALNAYLRNTKWNVIIREDLLRIDNKTCTLFRNKAVHLEVARYVHAYINDIAEVNSYFQLYHYIMQRIIMNERYEKSSGKVSEYFDAVNDEKKYNDRLLKLLCVPFGYCIPRFKNLSIEALFDRNEAAKFDKEKKKVSGN

SEQ ID NO: 781 - protein, 1152 aa, synthetic construct
MKVTKVDGISHKKYIEEGKLVKSTSEENRTSERLSELLSIRLDIYIKNPDNASEEENRIRRENLKKFFSNKVLHLKDSVLYLKNRKEKNAVQDKNYSEEDISEYDLKNKNSFSVLKKILLNEDVNSEELEIFRKDVEAKLNKINSLKYSFEENKANYQKINENNVEKVGGKSKRNIIYDYYRESAKRNDYINNVQEAFDKLYKKEDIEKLFFLIENSKKHEKYKIREYYHKIIGRKNDKENFAKIIYEEIQNVNNIKELIEKIPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQVGEIATSDFIARNRQNEAFLRNIIGVSSVAYFSLRNILETENENGITGRMRGKTVKNNKGEEKYVSGEVDKIYNENKQNEVKENLKMFYSYDFNMDNKNEIEDFFANIDEAISSIRHGIVHFNLELEGKDIFAFKNIAPSEISKKMFQNEINEKKLKLKIFKQLNSANVFNYYEKDVIIKYLKNTKFNFVNKNIPFVPSFTKLYNKIEDLRNTLKFFWSVPKDKEEKDAQIYLLKNIYYGEFLNKFVKNSKVFFKITNEVIKINKQRNQKTGHYKYQKFENIEKTVPVEYLAIIQSREMINNQDKEEKNTYIDFIQQIFLKGFIDYLNKNNLKYIESNNNNDNNDIFSKIKIKKDNKEKYDKILKNYEKHNRNKEIPHEINEFVREIKLGKILKYTENLNMFYLILKLLNHKELTNLKGSLEKYQSANKEETFSDELELINLLNLDNNRVTEDFELEANEIGKFLDFNENKIKDRKELKKFDTNKIYFDGENIIKHRAFYNIKKYGMLNLLEKIADKAKYKISLKELKEYSNKKNEIEKNYTMQQNLHRKYARPKKDEKFNDEDYKEYEKAIGNIQKYTHLKNKVEFNELNLLQGLLLKILHRLVGYTSIWERDLRFRLKGEFPENHYIEEIFNFDNSKNVKYKSGQIVEKYINFYKELYKDNVEKRSIYSDKKVKKLKQEKKDLYIRNYIAHFNYIPHAEISLLEVLENLRKLLSYDRKLKNAIMKSIVDILKEYGFVATFKIGADKKIEIQTLESEKIVHLKNLKKKKLMTDRNSEELCELVKVMFEYKALE

SEQ ID NO: 782 - protein, 1094 aa, synthetic construct
MNIPALVENQKKYFGTYSVMAMLNAQTVLDHIQKVADIEGEQNENNENLWFHPVMSHLYNAKNGYDKQPEKTMFIIERLQSYFPFLKIMAENQREYSNGKYKQNRVEVNSNDIFEVLKRAFGVLKMYRDLTNHYKTYEEKLNDGCEFLTSTEQPLSGMINNYYTVALRNMNERYGYKTEDLAFIQDKRFKFVKDAYGKKKSQVNTGFFLSLQDYNGDTQKKLHLSGVGIALLICLFLDKQYINIFLSRLPIFSSYNAQSEERRIIIRSFGINSIKLPKDRIHSEKSNKSVAMDMLNEVKRCPDELFTTLSAEKQSRFRIISDDHNEVLMKRSSDRFVPLLLQYIDYGKLFDHIRFHVNMGKLRYLLKADKTCIDGQTRVRVIEQPLNGFGRLEEAETMRKQENGTFGNSGIRIRDFENMKRDDANPANYPYIVDTYTHYILENNKVEMFINDKEDSAPLLPVIEDDRYVVKTIPSCRMSTLEIPAMAFHMFLFGSKKTEKLIVDVHNRYKRLFQAMQKEEVTAENIASFGIAESDLPQKILDLISGNAHGKDVDAFIRLTVDDMLTDTERRIKRFKDDRKSIRSADNKMGKRGFKQISTGKLADFLAKDIVLFQPSVNDGENKITGLNYRIMQSAIAVYDSGDDYEAKQQFKLMFEKARLIGKGTTEPHPFLYKVFARSIPANAVEFYERYLIERKFYLTGLSNEIKKGNRVDVPFIRRDQNKWKTPAMKTLGRIYSEDLPVELPRQMFDNEIKSHLKSLPQMEGIDFNNANVTYLIAEYMKRVLDDDFQTFYQWNRNYRYMDMLKGEYDRKGSLQHCFTSVEEREGLWKERASRTERYRKQASNKIRSNRQMRNASSEEIETILDKRLSNSRNEYQKSEKVIRRYRVQDALLFLLAKKTLTELADFDGERFKLKEIMPDAEKGILSEIMPMSFTFEKGGKKYTITSEGMKLKNYGDFFVLASDKRIGNLLELVGSDIVSKEDIMEEFNKYDQCRPEISSIVFNLEKWAFDTYPELSARVDREEKVDFKSILKILLNNKNINKEQSDILRKIRNAFDHNNYPDKGVVEIKALPEIAMSIKKAFGEYAIMKGSLQ

SEQ ID NO: 783 - protein, 1179 aa, synthetic construct
MTEQSERPYNGTYYTLEDKHFWAAFLNLARHNAYITLTHIDRQLAYSKADITNDQDVLSFKALWKNFDNDLERKSRLRSLILKHFSFLEGAAYGKKLFESKSSGNKSSKNKELTKKEKEELQANALSLDNLKSILFDFLQKLKDFRNYYSHYRHSGSSELPLFDGNMLQRLYNVFDVSVQRVKIDHEHNDEVDPHYHFNHLVRKGKKDRYGHNDNPSFKHHFVDGEGMVTEAGLLFFVSLFLEKRDAIWMQKKIRGFKGGTETYQQMTNEVFCRSRISLPKLKLESLRMDDWMLLDMLNELVRCPKPLYDRLREDDRACFRVPVDILPDEDDTDGGGEDPFKNTLVRHQDRFPYFALRYFDLKKVFTSLRFHIDLGTYHFAIYKKMIGEQPEDRHLTRNLYGFGRIQDFAEEHRPEEWKRLVRDLDYFETGDKPYISQTSPHYHIEKGKIGLRFMPEGQHLWPSPEVGTTRTGRSKYAQDKRLTAEAFLSVHELMPMMFYYFLLREKYSEEVSAERVQGRIKRVIEDVYAVYDAFARDEINTRDELDACLADKGIRRGHLPRQMIAILSQEHKDMEEKIRKKLQEMMADTDHRLDMLDRQTDRKIRIGRKNAGLPKSGVIADWLVRDMMRFQPVAKDASGKPLNNSKANSTEYRMLQRALALFGGEKERLTPYFRQMNLTGGNNPHPFLHETRWESHTNILSFYRSYLRARKAFLERIGRSDRVENRPFLLLKEPKTDRQTLVAGWKGEFHLPRGIFTEAVRDCLIEMGHDEVASYKEVGFMAKAVPLYFERACEDRVQPFYDSPFNVGNSLKPKKGRFLSKEERAEEWERGKERFRDLEAWSYSAARRIEDAFAGIEYASPGNKKKIEQLLRDLSLWEAFESKLKVRADRINLAKLKKEILEAQEHPYHDFKSWQKFERELRLVKNQDIITWMMCRDLMEENKVEGLDTGTLYLKDIRPNVQEQGSLNVLNRVKPMRLPVVVYRADSRGHVHKEEAPLATVYIEERDTKLLKQGNFKSFVKDRRLNGLFSFVDTGGLAMEQYPISKLRVEYELAKYQTARVCVFELTLRLEESLLTRYPHLPDESFREMLESWSDPLLAKWPELHGKVRLLIAVRNAFSHNQYPMYDEAVFSSIRKYDPSSPDAIEERMGLNIAHRLSEEVKQAKETVERIIQAGSLQ

SEQ ID NO: 784 - protein, 1111 aa, synthetic construct
MEKPLLPNVYTLKHKFFWGAFLNIARHNAFITICHINEQLGLKTPSNDDKIVDVVCETWNNILNNDHDLLKKSQLTELILKHFPFLTAMCYHPPKKEGKKKGHQKEQQKEKESEAQSQAEALNPSKLIEALEILVNQLHSLRNYYSHYKHKKPDAEKDIFKHLYKAFDASLRMVKEDYKAHFTVNLTRDFAHLNRKGKNKQDNPDFNRYRFEKDGFFTESGLLFFTNLFLDKRDAYWMLKKVSGFKASHKQREKMTTEVFCRSRILLPKLRLESRYDHNQMLLDMLSELSRCPKLLYEKLSEENKKHFQVEADGFLDEIEEEQNPFKDTLIRHQDRFPYFALRYLDLNESFKSIRFQVDLGTYHYCIYDKKIGDEQEKRHLTRTLLSFGRLQDFTEINRPQEWKALTKDLDYKETSNQPFISKTTPHYHITDNKIGFRLGTSKELYPSLEIKDGANRIAKYPYNSGFVAHAFISVHELLPLMFYQHLTGKSEDLLKETVRHIQRIYKDFEEERINTIEDLEKANQGRLPLGAFPKQMLGLLQNKQPDLSEKAKIKIEKLIAETKLLSHRLNTKLKSSPKLGKRREKLIKTGVLADWLVKDFMRFQPVAYDAQNQPIKSSKANSTEFWFIRRALALYGGEKNRLEGYFKQTNLIGNTNPHPFLNKFNWKACRNLVDFYQQYLEQREKFLEAIKNQPWEPYQYCLLLKIPKENRKNLVKGWEQGGISLPRGLFTEAIRETLSEDLMLSKPIRKEIKKHGRVGFISRAITLYFKEKYQDKHQSFYNLSYKLEAKAPLLKREEHYEYWQQNKPQSPTESQRLELHTSDRWKDYLLYKRWQHLEKKLRLYRNQDVMLWLMTLELTKNHFKELNLNYHQLKLENLAVNVQEADAKLNPLNQTLPMVLPVKVYPATAFGEVQYHKTPIRTVYIREEHTKALKMGNFKALVKDRRLNGLFSFIKEENDTQKHPISQLRLRRELEIYQSLRVDAFKETLSLEEKLLNKHTSLSSLENEFRALLEEWKKEYAASSMVTDEHIAFIASVRNAFCHNQYPFYKEALHAPIPLFTVAQPTTEEKDGLGIAEALLKVLREYCEIVKSQIGSSLQKKLEELELGSS

SEQ ID NO: 785 - RNA, 48 nt, synthetic construct
  source 1..48: mol_type = other RNA
ttgtgtttctacttatagatggctaaaatctgagtttagaaaatgcaa

SEQ ID NO: 786 - RNA, 10 nt, synthetic construct
  source 1..10: mol_type = other RNA
aatttttgga

SEQ ID NO: 787 - RNA, 19 nt, synthetic construct
  source 1..19: mol_type = other RNA
ctgaatgcctgcgagcatc

SEQ ID NO: 788 - protein, 621 aa, synthetic construct
MELDPDDVFRDEDEDPENDFFQEKEASKEFVVYLIDASPKMFCSTCPSEEEDKQESHFHIAVSCIAQSLKAHIINRSNDEIAICFFNTREKKNLQDLNGVYVFNVPERDSIDRPTARLIKEFDLIEESFDKEIGSQTGIVSDSRENSLYSALWVAQALLRKGSLKTADKRMFLFTNEDDPFGSMRISVKEDMTRTTLQRAKDAQDLGISIELLPLSQPDKQFNITLFYKDLIGLNSDELTEFMPSVGQKLEDMKDQLKKRVLAKRIAKRITFVICDGLSIELNGYALLRPAIPGSITWLDSTTNLPVKVERSYICTDTGAIMQDPIQRIQPYKNQNIMFTVEELSQVKRISTGHLRLLGFKPLSCLKDYHNLKPSTFLYPSDKEVIGSTRAFIALHRSMIQLERFAVAFYGGTTPPRLVALVAQDEIESDGGQVEPPGINMIYLPYANDIRDIDELHSKPGVAAPRASDDQLKKASALMRRLELKDFSVCQFANPALQRHYAILQAIALDENELRETRDETLPDEEGMNRPAVVKAIEQFKQSIYGDDPDEESDSGAKEKSKKRKAGDADDGKYDYIELAKTGKLKDLTVVELKTYLTANNLLVSGKKEVLINRILTHIGK

SEQ ID NO: 789 - protein, 629 aa, synthetic construct
MSSESTTFIVDVSPSMMKNNNVSKSMAYLEYTLLNKSKKSRKTDWISCYLANCPVSENSQEIPNVFQIQSFLAPVTTTATIGFIKRLKQYCDQHSHDSSNEGLQSMIQCLLVVSLDIKQQFQARKILKQIVVFTDNLDDLDITDEEIDLLTEELSTRIILIDCGKDTQEERKKSNWLKLVEAIPNSRIYNMNELLVEITSPATSVVKPVRVFSGELRLGADILSTQTSNPSGSMQDENCLCIKVEAFPATKAVSGLNRKTAVEVEDSQKKERYVGVKSIIEYEIHNEGNKKNVSEDDQSGSSYIPVTISKDSVTKAYRYGADYVVLPSVLVDQTVYESFPGLDLRGFLNREALPRYFLTSESSFITADTRLGCQSDLMAFSALVDVMLENRKIAVARYVSKKDSEVNMCALCPVLIEHSNINSEKKFVKSLTLCRLPFAEDERVTDFPKLLDRTTTSGVPLKKETDGHQIDELMEQFVDSMDTDELPEIPLGNYYQPIGEVTTDTTLPLPSLNKDQEENKKDPLRIPTVFVYRQQQVLLEWIHQLMINDSREFEIPELPDSLKNKISPYTHKKFDSTKLVEVLGIKKVDKLKLDSELKTELEREKIPDLETLLKRGEQHSRGSPNNSNN

SEQ ID NO: 790 - protein, 70 aa, synthetic construct
GSVIDVSSQRVNVQRPLDALGNSLNSPVIIKLKGDREFRGVLKSFDLHMNLVLNDAEELEDGEVTRRLGT

SEQ ID NO: 791 - protein, 62 aa, synthetic construct
MKSIRCKNCNKLLFKADSFDHIEIRCPRCKRHIIMLNACEHPTEKHCGKREKITHSDETVRY

SEQ ID NO: 792 - protein, 193 aa, Felis catus
MNPLQEVIFCRQFGNQHRVPKPYYRRKTYLCYQLKLPEGTLIHKDCLRNKKKRHAEMCFIDKIKALTRDTSQRFEIICYITWSPCPFCAEELVAFVKDNPHLSLRIFASRLYVHWRWKYQQGLRHLHASGIPVAVMSLPEFEDCWRNFVDHQDRSFQPWPNLDQYSKSIKRRLGKILTPLNDLRNDFRNLKLE

SEQ ID NO: 793 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 794 - protein, 396 aa, Cricetulus longicaudatus
MGPFCLGCSHRKCYSPIRNLISQETFKFHFKNLGYAKGRKDTFLCYEVTRKDCDSPVSLHHGVFKNKGNIHAEVCFLYWFHDKVLKVLSPREEFKITWYMSWSPCFECAEQIVRFLATHHYLSLDIFSSRLYNVQDPETQQNLCRLVQEGAQVAAMDLYEFKKCWKKFVTMVAGDSGLGKRLLTNFRYQDSKLQEILRRMDPLSEEEFYSQFYNQRVKHLCYYHRMKPYLCYQLEQFNGQAPLKGCLLSEKGKQHAEILFLDKIRSMELSQVTITCYLTWSPCPNCAWRLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 795 - protein, 429 aa, Rattus norvegicus
MGPFCLGCSHRKCYSPIRNLISQETFKFHFKNLRYAIDRKDTFLCYEVTRKDCDSPVSLHHGVFKNKDNIHAEICFLYWFHDKVLKVLSPREEFKITWYMSWSPCFECAEQVLRFLATHHNLSLDIFSSRLYNIRDPENQQNLCRLVQEGAQVAAMDLYEFKKCWKKFVDNGGRRFRPWKKLLTNFRYQDSKLQEILRPCYIPVPSSSSSTLSNICLTKGLPETRFCVERRRVHLLSEEEFYSQFYNQRVKHLCYYHGVKPYLCYQLEQFNGQAPLKGCLLSEKGKQHAEILFLDKIRSMELSQVIITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLHRIKESWGLQDLVNDFGNLQLGPPMS

SEQ ID NO: 796 - protein, 211 aa, Pongo pygmaeus
MALLTAKTFSLQFNNKRRIKRPYYPRKALLCYQLTPQNGSTPTRGYFKNKKKCHAEIRFINEIKSMGLDETQCYQVTCYLTWSPCPSCVRELVAFIKAHDHLNLRIFASRLYCHWCRRQQEGLRLLCGSQVPVEVMGSREFADCWENFVDHEKPLSFNPSEMLEELDKNSRAIKRRLERIKQSWSVDVLENGLRSLQLGPVSSSLSRSNSR

SEQ ID NO: 797 - protein, 192 aa, Felis catus
MEPWRPSPRNPMDRIDPNTFRFHFPNLLYASGRKLCYLCFQVETEDYFSCDDSDRGVFRNKVHPWARCHAEQCFLSWFRDQYPYRDEYYNVTWFLSWSPCPTCAEEVVEFLEEYRNLTLSIFTSRLYYFWDPNYQEGLCKLWDAGVQLDIMSCDDFKHCWDNFVDHKGMRFQRRNLLKDYDFLAAELQEILR

SEQ ID NO: 798 - protein, 200 aa, Homo sapiens
MALLTAETFRLQFNNKRRLRRPYYPRKALLCYQLTPQNGSTPTRGYFENKKKCHAEICFINEIKSMGLDETQCYQVTCYLTWSPCSSCAWELVDFIKAHDHLNLGIFASRLYYHWCKPQQKGLRLLCGSQVPVEVMGFPEFADCWENFVDHEKPLSFNPYKMLEELDKNSRAIKRRLERIKIPGVRAQGRYMDILCDAEV

SEQ ID NO: 799 - protein, 370 aa, Macaca mulatta
MVEPMDPRTFVSNFNNRPILSGLNTVWLCCEVKTKDPSGPPLDAKIFQGKVYSKAKYHPEMRFLRWFHKWRQLHHDQEYKVTWYVSWSPCTRCANSVATFLAKDPKVTLTIFVARLYYFWKPDYQQALRILCQKRGGPHATMKIMNYNEFQDCWNKFVDGRGKPFKPRNNLPKHYTLLQATLGELLRHLMDPGTFTSNFNNKPWVSGQHETYLCYKVERLHNDTWVPLNQHRGFLRNQAPNIHGFPKGRHAELCFLDLIPFWKLDGQQYRVTCFTSWSPCFSCAQEMAKFISNNEHVSLCIFAARIYDDQGRYQEGLRALHRDGAKIAMMNYSEFEYCWDTFVDRQGRPFQPWDGLDEHSQALSGRLRAI

SEQ ID NO: 800 - protein, 384 aa, Pan troglodytes
MKPHFRNPVERMYQDTFSDNFYNRPILSHRNTVWLCYEVKTKGPSRPPLDAKIFRGQVYSKLKYHPEMRFFHWFSKWRKLHRDQEYEVTWYISWSPCTKCTRDVATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTSNFNNELWVRGRHETYLCYEVERLHNDTWVLLNQRRGFLCNQAPHKHGFLEGRHAELCFLDVIPFWKLDLHQDYRVTCFTSWSPCFSCAQEMAKFISNNKHVSLCIFAARIYDDQGRCQEGLRTLAKAGAKISIMTYSEFKHCWDTFVDHQGCPFQPWDGLEEHSQALSGRLRAILQNQGN

SEQ ID NO: 801 - protein, 377 aa, Chlorocebus aethiops
MNPQIRNMVEQMEPDIFVYYFNNRPILSGRNTVWLCYEVKTKDPSGPPLDANIFQGKLYPEAKDHPEMKFLHWFRKWRQLHRDQEYEVTWYVSWSPCTRCANSVATFLAEDPKVTLTIFVARLYYFWKPDYQQALRILCQERGGPHATMKIMNYNEFQHCWNEFVDGQGKPFKPRKNLPKHYTLLHATLGELLRHVMDPGTFTSNFNNKPWVSGQRETYLCYKVERSHNDTWVLLNQHRGFLRNQAPDRHGFPKGRHAELCFLDLIPFWKLDDQQYRVTCFTSWSPCFSCAQKMAKFISNNKHVSLCIFAARIYDDQGRCQEGLRTLHRDGAKIAVMNYSEFEYCWDTFVDRQGRPFQPWDGLDEHSQALSGRLRAI

SEQ ID NO: 802 - protein, 373 aa, Homo sapiens
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEGQPFMPWYKFDDNYAFLHRTLKEILRNPMEAMYPHIFYFHFKNLRKAYGRNESWLCFTMEVVKHHSPVSWKRGVFRNQVDPETHCHAERCFLSWFCDDILSPNTNYEVTWYTSWSPCPECAGEVAEFLARHSNVNLTIFTARLYYFWDTDYQEGLRSLSQEGASVEIMGYKDFKYCWENFVYNDDEPFKPWKGLKYNFLFLDSKLQEILE

SEQ ID NO: 803 - protein, 384 aa, Homo sapiens
MKPHFRNTVERMYRDTFSYNFYNRPILSRRNTVWLCYEVKTKGPSRPPLDAKIFRGQVYSELKYHPEMRFFHWFSKWRKLHRDQEYEVTWYISWSPCTKCTRDMATFLAEDPKVTLTIFVARLYYFWDPDYQEALRSLCQKRDGPRATMKIMNYDEFQHCWSKFVYSQRELFEPWNNLPKYYILLHIMLGEILRHSMDPPTFTFNFNNEPWVRGRHETYLCYEVERMHNDTWVLLNQRRGFLCNQAPHKHGFLEGRHAELCFLDVIPFWKLDLDQDYRVTCFTSWSPCFSCAQEMAKFISKNKHVSLCIFTARIYDDQGRCQEGLRTLAEAGAKISIMTYSEFKHCWDTFVDHQGCPFQPWDGLDEHSQDLSGRLRAILQNQEN

SEQ ID NO: 804 - protein, 190 aa, Homo sapiens
MNPQIRNPMKAMYPGTFYFQFKNLWEANDRNETWLCFTVEGIKRRSVVSWKTGVFRNQVDSETHCHAERCFLSWFCDDILSPNTKYQVTWYTSWSPCPDCAGEVAEFLARHSNVNLTIFTARLYYFQYPCYQEGLRSLSQEGVAVEIMDYEDFKYCWENFVYNDNEPFKPWKGLKTNFRLLKRRLRESLQ

SEQ ID NO: 805 - protein, 382 aa, Homo sapiens
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVTIMDYEEFAYCWENFVYNEGQQFMPWYKFDENYAFLHRTLKEILRYLMDPDTFTFNFNNDPLVLRRRQTYLCYEVERLDNGTWVLMDQHMGFLCNEAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFEYCWDTFVYRQGCPFQPWDGLEEHSQALSGRLRAILQNQGN

SEQ ID NO: 806 - protein, 210 aa, Macaca mulatta
MALLTAKTFSLQFNNKRRVNKPYYPRKALLCYQLTPQNGSTPTRGHLKNKKKDHAEIRFINKIKSMGLDETQCYQVTCYLTWSPCPSCAGELVDFIKAHRHLNLRIFASRLYYHWRPNYQEGLLLLCGSQVPVEVMGLPEFTDCWENFVDHKEPPSFNPSEKLEELDKNSQAIKRRLERIKSRSVDVLENGLRSLQLGPVTPSSSIRNSR

SEQ ID NO: 807 - protein, 386 aa, Homo sapiens
MNPQIRNPMERMYRDTFYDNFENEPILYGRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRLHKAGARVKIMDYEDFAYCWENFVCNEGQPFMPWYKFDDNYASLHRTLKEILRNPMEAMYPHIFYFHFKNLLKACGRNESWLCFTMEVTKHHSAVFRKRGVFRNQVDPETHCHAERCFLSWFCDDILSPNTNYEVTWYTSWSPCPECAGEVAEFLARHSNVNLTIFTARLCYFWDTDYQEGLCSLSQEGASVKIMGYKDFVSCWKNFVYSDDEPFKPWKGLQTNFRLLKRRLREILQ

SEQ ID NO: 808 - protein, 440 aa, Mus musculus
MQPQRLGPRAGMGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSSSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 809 - protein, 229 aa, Rattus norvegicus [sequence omitted]

SEQ ID NO: 810 - protein, 236 aa, Homo sapiens
MTSEKGPSTGDPTLRRRIEPWEFDVFYDPRELRKEACLLYEIKWGMSRKIWRSSGKNTTNHVEVNFIKKFTSERDFHPSMSCSITWFLSWSPCWECSQAIREFLSRHPGVTLVIYVARLFWHMDQQNRQGLRDLVNSGVTIQIMRASEYYHCWRNFVNYPPGDEAHWPQYPPLWMMLYALELHCIILSLPPCLKISRRWQNHLTFFRLHLQNCHYQTIPPHILLATGLIHPSVAWR

SEQ ID NO: 811 - protein, 236 aa, Oryctolagus cuniculus
MASEKGPSNKDYTLRRRIEPWEFEVFFDPQELRKEACLLYEIKWGASSKTWRSSGKNTTNHVEVNFLEKLTSEGRLGPSTCCSITWFLSWSPCWECSMAIREFLSQHPGVTLIIFVARLFQHMDRRNRQGLKDLVTSGVTVRVMSVSEYCYCWENFVNYPPGKAAQWPRYPPRWMLMYALELYCIILGLPPCLKISRRHQKQLTFFSLTPQYCHYKMIPPYILLATGLLQPSVPWR

SEQ ID NO: 812 - protein, 229 aa, Mus musculus
MSSETGPVAVDPTLRRRIEPHEFEVFFDPRELRKETCLLYEINWGGRHSVWRHTSQNTSNHVEVNFLEKFTTERYFRPNTRCSITWFLSWSPCGECSRAITEFLSRHPYVTLFIYIARLYHHTDQRNRQGLRDLISSGVTIQIMTEQEYCYCWRNFVNYPPSNEAYWPRYPHLWVKLYVLELYCIILGLPPCLKILRRKQPQLTFFTITLQTCHYQRIPPHLLWATGLK

SEQ ID NO: 813 - protein, 229 aa, Mesocricetus auratus
MSSETGPVVVDPTLRRRIEPHEFDAFFDQGELRKETCLLYEIRWGGRHNIWRHTGQNTSRHVEINFIEKFTSERYFYPSTRCSIVWFLSWSPCGECSKAITEFLSGHPNVTLFIYAARLYHHTDQRNRQGLRDLISRGVTIRIMTEQEYCYCWRNFVNYPPSNEVYWPRYPNLWMRLYALELYCIHLGLPPCLKIKRRHQYPLTFFRLNLQSCHYQRIPPHILWATGFI

SEQ ID NO: 814 - protein, 235 aa, Monodelphis domestica
MNSKTGPSVGDATLRRRIKPWEFVAFFNPQELRKETCLLYEIKWGNQNIWRHSNQNTSQHAEINFMEKFTAERHFNSSVRCSITWFLSWSPCWECSKAIRKFLDHYPNVTLAIFISRLYWHMDQQHRQGLKELVHSGVTIQIMSYSEYHYCWRNFVDYPQGEEDYWPKYPYLWIMLYVLELHCIILGLPPCLKISGSHSNQLALFSLDLQDCHYQKIPYNVLVATGLVQPFVTWR

SEQ ID NO: 815 - protein, 236 aa, Pongo pygmaeus
MTSEKGPSTGDPTLRRRIESWEFDVFYDPRELRKETCLLYEIKWGMSRKIWRSSGKNTTNHVEVNFIKKFTSERRFHSSISCSITWFLSWSPCWECSQAIREFLSQHPGVTLVIYVARLFWHMDQRNRQGLRDLVNSGVTIQIMRASEYYHCWRNFVNYPPGDEAHWPQYPPLWMMLYALELHCIILSLPPCLKISRRWQNHLAFFRLHLQNCHYQTIPPHILLATGLIHPSVTWR

SEQ ID NO: 816 - protein, 224 aa, Bos taurus
MAQKEEAAAAAEPASQNGEEVENLEDPEKLKELIELPPFEIVTGERLPAHYFKFQFRNVEYSSGRNKTFLCYVVEAQSKGGQVQASRGYLEDEHATNHAEEAFFNSIMPTFDPALRYMVTWYVSSSPCAACADRIVKTLNKTKNLRLLILVGRLFMWEEPEIQAALRKLKEAGCRLRIMKPQDFEYIWQNFVEQEEGESKAFEPWEDIQENFLYYEEKLADILK

SEQ ID NO: 817 - protein, 224 aa, Mus musculus
MAQKEEAAEAAAPASQNGDDLENLEDPEKLKELIDLPPFEIVTGVRLPVNFFKFQFRNVEYSSGRNKTFLCYVVEVQSKGGQAQATQGYLEDEHAGAHAEEAFFNTILPAFDPALKYNVTWYVSSSPCAACADRILKTLSKTKNLRLLILVSRLFMWEEPEVQAALKKLKEAGCKLRIMKPQDFEYIWQNFVEQEEGESKAFEPWEDIQENFLYYEEKLADILK

SEQ ID NO: 818 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 819 - protein, 224 aa, Pongo pygmaeus
MAQKEEAAAATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEELEIQDALKKLKEAGCKLRIMKPQDFEYVWQNFVEQEEGESKAFQPWEDIQENFLYYEEKLADILK

SEQ ID NO: 820 - protein, 367 aa, Homo sapiens
MEPIYEEYLANHGTIVKPYYWLSFSLDCSNCPYHIRTGEEARVSLTEFCQIFGFPYGTTFPQTKHLTFYELKTSSGSLVQKGHASSCTGNYIHPESMLFEMNGYLDSAIYNNDSIRHIILYSNNSPCNEANHCCISKMYNFLITYPGITLSIYFSQLYHTEMDFPASAWNREALRSLASLWPRVVLSPISGGIWHSVLHSFISGVSGSHVFQPILTGRALADRHNAYEINAITGVKPYFTDVLLQTKRNPNTKAQEALESYPLNNAFPGQFFQMPSGQLQPNLPPDLRAPVVFVLVPLRDLPPMHMGQNPNKPRNIVRHLNMPQMSFQETKDLGRLPTGRSVEIVEITEQFASSKEADEKKKKKGKK

SEQ ID NO: 821 - protein, 388 aa, Rattus norvegicus
MEPLYEEYLTHSGTIVKPYYWLSVSLNCTNCPYHIRTGEEARVPYTEFHQTFGFPWSTYPQTKHLTFYELRSSSGNLIQKGLASNCTGSHTHPESMLFERDGYLDSLIFHDSNIRHIILYSNNSPCDEANHCCISKMYNFLMNYPEVTLSVFFSQLYHTENQFPTSAWNREALRGLASLWPQVTLSAISGGIWQSILETFVSGISEGLTAVRPFTAGRTLTDRYNAYEINCITEVKPYFTDALHSWQKENQDQKVWAASENQPLHNTTPAQWQPDMSQDCRTPAVFMLVPYRDLPPIHVNPSPQKPRTVVRHLNTLQLSASKVKALRKSPSGRPVKKEEARKGSTRSQEANETNKSKWKKQTLFIKSNICHLLEREQKKIGILSSWSV

SEQ ID NO: 822 - protein, 374 aa, Mus musculus
MEPLYEEILTQGGTIVKPYYWLSLSLGCTNCPYHIRTGEEARVPYTEFHQTFGFPWSTYPQTKHLTFYELRSSSKNLIQKGLASNCTGSHNHPEAMLFEKNGYLDAVIFHNSNIRHIILYSNNSPCNEAKHCCISKMYNFLMNYPEVTLSVFFSQLYHTEKQFPTSAWNRKALQSLASLWPQVTLSPICGGLWHAILEKFVSNISGSTVPQPFIAGRILADRYNTYEINSIIAAKPYFTDGLLSRQKENQNREAWAAFEKHPLGSAAPAQRQPTRGQDPRTPAVLMLVSNRDLPPIHVGSTPQKPRTVVRHLNMLQLSSFKVKDVKKPPSGRPVEEVEVMKESARSQKANKKNRSQWKKQTLVIKPRICRLLER

SEQ ID NO: 823 - protein, 363 aa, Macaca fascicularis
MEPTYEEYLANHGTIVKPYYWLSFSLDCSNCPYHIRTGEEARVSLTEFCQIFGFPYGTTYPQTKHLTFYELKTSSGSLVQKGHASSCTGNYIHPESMLFEMNGYLDSAIYNNDSIRHIILYCNNSPCNEANHCCISKVYNFLITYPGITLSIYFSQLYHTEMDFPASAWNREALRSLASLWPRVVLSPISGGIWHSVLHSFVSGVSGSHVFQPILTGRALTDRYNAYEINAITGVKPFFTDVLLHTKRNPNTKAQMALESYPLNNAFPGQSFQMTSGIPPDLRAPVVFVLLPLRDLPPMHMGQDPNKPRNIIRHLNMPQMSFQETKDLERLPTRRSVETVEITERFASSKQAEEKTKKKKGKK

SEQ ID NO: 824 - protein, 198 aa, Homo sapiens [sequence omitted]

SEQ ID NO: 825 - protein, 198 aa, Mus musculus
MDSLLMKQKKFLYHFKNVRWAKGRHETYLCYVVKRRDSATSCSLDFGHLRNKSGCHVELLFLRYISDWDLDPGRCYRVTWFTSWSPCYDCARHVAEFLRWNPNLSLRIFTARLYFCEDRKAEPEGLRRLHRAGVQIGIMTFKDYFYCWNTFVENRERTFKAWEGLHENSVRLTRQLRRILLPLYEVDDLRDAFRMLGF

SEQ ID NO: 826 - protein, 198 aa, Canis lupus
MDSLLMKQRKFLYHFKNVRWAKGRHETYLCYVVKRRDSATSFSLDFGHLRNKSGCHVELLFLRYISDWDLDPGRCYRVTWFTSWSPCYDCARHVADFLRGYPNLSLRIFAARLYFCEDRKAEPEGLRRLHRAGVQIAIMTFKDYFYCWNTFVENREKTFKAWEGLHENSVRLSRQLRRILLPLYEVDDLRDAFRTLGL

SEQ ID NO: 827 - protein, 199 aa, Bos taurus
MDSLLKKQRQFLYQFKNVRWAKGRHETYLCYVVKRRDSPTSFSLDFGHLRNKAGCHVELLFLRYISDWDLDPGRCYRVTWFTSWSPCYDCARHVADFLRGYPNLSLRIFTARLYFCDKERKAEPEGLRRLHRAGVQIAIMTFKDYFYCWNTFVENHERTFKAWEGLHENSVRLSRQLRRILLPLYEVDDLRDAFRTLGL

SEQ ID NO: 828 - protein, 152 aa, Rhizobium sp.
  source 1..152: note = Agrobacterium fabrum
MAERTHFMELALVEARSAGERDEVPIGAVLVLDGRVIARSGNRTRELNDVTAHAEIAVIRMACEALGQERLPGADLYVTLEPCTMCAAAISFARIRRLYYGAQDPKGGAVESGVRFFSQPTCHHAPDVYSGLAESESAEILRQFFREKRLDD

SEQ ID NO: 829 - protein, 1307 aa, Arabidopsis thaliana
MFNTYTNSLQWPIRSRNQQDYCSLLPERSESYKLSKAYTSSRCYCVSSRSSCCCCCSTPSSSSFVKPKVLINPGFVLYGVRQSTLIQWPSFQRRLLVGGGRLMGCEVYSSCDGIRRKNRSFKLRCLEESDECCGGRSCSDDVEAMISFLSEELIDEERKWNLVSRVKEKKKVGNVRKVSVEGSNSYGNGRVSQRVKKPEGFGRRKEIKEDVKLNERYDCEHCGRRKKSSELESESRRGSKLVTGEYIGKSYRGDEEREVRPRRRKSSSCSSYYSLASSGEFESDTEDQEEDVEIYRENVRSSEKKVVDQSAKRLKSRKEASQMHSRKKRDESSTGVDSRYQKQIFEEGENSNQAVTLNQRRRKKFSQTENRVSESTGNYEEDMEIHEVHVNDAETSSQNQKLFNEREDYRVHSIRNDSGNENIESSQHQLKERLETRYSSEDRVSEMRRRTKYSSSQEEGINVLQNFPEVTNNQQPLVEERISKQAGTRRTTEHISESSEIHDIDIRNTYVSQREDQIRNQEVHAGLVSGLQSERKQQDYHIEHNPLQTTQSDRTSVSVSHTSDAVRYTEIQRKSEKRLIGQGSTTAVQSDSKVEKNGAQKEDSRLDHANSKKDGQTTLGLQSYQSKLSEEASSSQSSLMASRTKLQLVDLVSEEMQGSETTLIPPSSQLVSRRSGQSYRTGGVSIQEISHGTSESGYTTAFEHPRAGASVNSQSAGELMGFTSHEDAMGSAHRLEQASEKYVGEFVKKAKHGVINPETEEQRAESNQLKRRDSRRSSGGSGAKGPSDEMWVTDSAQGTPHPGATEGNAAVGNAIFKRNGRSLWNVIADIARLRWGSRAGSPDSSAKPAGRSSPNESVSSATWFSGREHDGSSDDNTKGDKVLPQEAPSLHQVEVGQTSPRSQSEYPGTTKLKQRSERHEGVVSSPSSTILEGGSVSNRMSSTSGNQIVGVDEEEGGNFEFRLPETALTEVPMKLPSRNLIRSPPIKESSESSLTEASSDQNFTVGEGRRYPRMDAGQNPLLFPGRNLRSPAVMEPPVPRPRMVSGSSSLREQVEQQQPLSAKSQEETGSVSADSALIQRKLQRNKQVVRDSFEEWEEAYKVEAERRTVDEIFMREALVEAKKAADTWEVPVGAVLVHDGKIIARGYNLVEELRDSTAHAEMICIREGSKALRSWRLADTTLYVTLEPCPMCAGAILQARVNTLVWGAPNKLLGADGSWIRLFPGGEGNGSEASEKPPPPVHPFHPKMTIRRGVLESECAQTMQQFFQLRRKKKDKNSDPPTPTDHHHHHLPKLLNKMHQVLPFFCL

SEQ ID NO: 830 - protein, 151 aa, Aquifex sp.
  source 1..151: note = Aquifex aeolicus
MGKEYFLKVALREAKRAFEKGEVPVGAIIVKEGEIISKAHNSVEELKDPTAHAEMLAIKEACRRLNTKYLEGCELYVTLEPCIMCSYALVLSRIEKVIFSALDKKHGGVVSVFNILDEPTLNHRVKWEYYPLEEASELLSEFFKKLRNNII

SEQ ID NO: 831 - protein, 171 aa, Streptococcus pyogenes
MPYSLEEQTYFMQEALKEAEKSLQKAEIPIGCVIVKDGEIIGRGHNAREESNQAIMHAEMMAINEANAHEGNWRLLDTTLFVTIEPCVMCSGAIGLARIPHVIYGASNQKFGGADSLYQILTDERLNHRVQVERGLLAADCANIMQTFFRQGRERKKIAKHLIKEQSDPFD

SEQ ID NO: 832 - protein, 161 aa, Bacillus subtilis
MTQDELYMKEAIKEAKKAEEKGEVPIGAVLVINGEIIARAHNLRETEQRSIAHAEMLVIDEACKALGTWRLEGATLYVTLEPCPMCAGAVVLSRVEKVVFGAFDPKGGCSGTLMNLLQEERFNHQAEVVSGVLEEECGGMLSAFFRELRKKKKAARKNLSE

SEQ ID NO: 833 - protein, 173 aa, Haemophilus influenzae
MDAAKVRSEFDEKMMRYALELADKAEALGEIPVGAVLVDDARNIIGEGWNLSIVQSDPTAHAEIIALRNGAKNIQNYRLLNSTLYVTLEPCTMCAGAILHSRIKRLVFGASDYKTGAIGSRFHFFDDYKMNHTLEVTSGVLAEECSQKLSTFFQKRREEKKIEKALLKSLSDK

SEQ ID NO: 834 - protein, 161 aa, Buchnera aphidicola
MKYEKDKNWMKIALKYAYYAKEKGEIPIGAILVFKERIIGIGWNSSISKNDPTAHAEIIALRGAGKKIKNYRLLNTTLYVTLQPCIMCCGAIIQSRIKRLVFGANCNSSDHRFSLKNLFCDPQKDYKLDIKKNVMQRECSDILINFFQKKRKNKIHICKKI

SEQ ID NO: 835 - protein, 167 aa, Escherichia coli
MSEVEFSHEYWMRHALTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 836 - protein, 171 aa, Streptococcus pyogenes
MPYSLEEQTYFMQEALKEAEKSLQKAEIPIGCVIVKDGEIIGRGHNAREESNQAIMHAEMMAINEANAHEGNWRLLDTTLFVTIEPCVMCSGAIGLARIPHVIYGASNQKFGGVDSLYQILTDERLNHRVQVERGLLAADCANIMQTFFRQGRERKKIAKHLIKEQSDPFD

SEQ ID NO: 837 - protein, 145 aa, Rickettsia bellii
MREALKQAEIAFSKNEVPVGAVIVDRENQKIISKSYNNTEEKNNALYHAEIIAINEACRIISSKNLSDYDIYVTLEPCAMCAAAIAHSRLKRLFYGASDSKHGAVESNLRYFNSKACFHRPEIYSGIFAEDSALLMKGFFKKIRD

SEQ ID NO: 838 - protein, 144 aa, Rickettsia felis
MEQALKQAGIAFDKNEVPVGAVIVDRLNQKIIVSSHNNTEEKNNALYHAEIIAINEACNLISSKNLNDYDIYVTLEPCAMCAAAIAHSRLKRLFYGASDSKHGAVESNLRYFNSSVCFYRPEIYSGILAEDSRLLMKEFFKRIR

SEQ ID NO: 839 - protein, 172 aa, Salmonella enterica
  source 1..172: note = Salmonella typhimurium
MSDVELDHEYWMRHALTLAKRAWDEREVPVGAVLVHNHRVIGEGWNRPIGRHDPTAHAEIMALRQGGLVLQNYRLLDTTLYVTLEPCVMCAGAMVHSRIGRVVFGARDAKTGAAGSLIDVLHHPGMNHRVEIIEGVLRDECATLLSDFFRMRRQEIKALKKADRAEGAGPAV

SEQ ID NO: 840 - protein, 167 aa, Escherichia coli
MSEVEFSHEYWMRHAMTLAKRAWDEREVPVGAVLVHNNRVIGEGWNRPIGRHDPTAHAEIMALRQGGLVMQNYRLIDATLYVTLEPCVMCAGAMIHSRIGRVVFGARDAKTGAAGSLMDVLHHPGMNHRVEITEGILADECAALLSDFFRMRRQEIKAQKKAQSSTD

SEQ ID NO: 841 - protein, 151 aa, Buchnera aphidicola
  source 1..151: note = Buchnera aphidicola subsp. Baizongia pistaciae
MKSNRDSYWMKIALKYAYYAEENGEVPIGAILVFQEKIIGTGWNSVISQNDSTAHAEIIALREAGRNIKNYRLVNTTLYVTLQPCMMCCGAIINSRIKRLVFGASYKDLKKNPFLKKIFINLEKNKLKIKKHIMRNECAKILSNFFKNKRF

SEQ ID NO: 842 - protein, 159 aa, Streptococcus pyogenes
MPYSLEEQTYFMQEALKEAEKSLQKAEIPIGCVIVKDGEIIGRGHNAREESNQAIMHAEMMAINEANAHEGNWRLLDTTLFVTIEPCVMCSGAIGLARIPHVIYGASNQKFGGADSLYQILTDERLNHRVQVERGLLAADCANIMQTFFRQGRERKKNS

SEQ ID NO: 843 - protein, 148 aa, Rickettsia prowazekii
MEQALKQARLAFDKNEVPVGVVIVCRLNQKIIVSSHNNIEEKKNPLCHAEIIAINTACNLISSKNLNDYDIYVTLEPCAMCASAISHSRLKRLFYGASDSKHGAVESNLRYFNSNSCFYRPEIYSGILSEHSRFLMQEFFQRIRSAID

SEQ ID NO: 844 - protein, 148 aa, Rickettsia typhi
MEQALKQARLAFDKNEVPVGVVIVYRLNQKIIVSSHNNIEEKNNALCHAEIIAINEACNLISSKNLNDYDIYVTLEPCAMCASAISHSRLKRLFYGASDSKQGAVESNLRYFNSSACFHRPEIYSGILSEHSRFLMKEFFQKMRSTID

SEQ ID NO: 845 - protein, 162 aa, Buchnera aphidicola
  source 1..162: note = Buchnera aphidicola subsp. Schizaphis graminum
MHDSDKYFMKCAIFLAKISEMIGEVPVGAVLVFNNTIIGKGLNSSILNHDPTAHAEIKALRNGAKFLKNYRLLHTTLYVTLEPCIMCYGAIIHSRISRLVFGAKYKNLQKYICCKNHFFINKNFRKISITQEVLESECSNLLSSFFKRKRKIATKYFNNNII

SEQ ID NO: 846 - protein, 153 aa, Rickettsia conorii
MEQALKQAKIAFDKNEVPVGAVVVDRLHQKIIASTHNNTEEKNNALYHAEIIAINEACNLISSKNLNDYDIYVTLEPCAMCAAAIAHSRLKRLFYGASDSKHGVVESNLRYFNSSACFHRPEIYSGILAEDSGLLMKEFFKRIRTVISSHRMT

SEQ ID NO: 847 - protein, 156 aa, Staphylococcus aureus
MTNDIYFMTLAIEEAKKAAQLGEVPIGAIITKDDEVIARAHNLRETLQQPTAHAEHIAIERAAKVLGSWRLEGCTLYVTLEPCVMCAGTIVMSRIPRVVYGADDPKGGCSGSLMNLLQQSNFNHRAIVDKGVLKEACSTLLTTFFKNLRANKKSTN

SEQ ID NO: 848 - protein, 420 aa, Arabidopsis thaliana
MEEDWGKTVSEKVISAYMSLPKKGKPQGREVTVLSAFLVSSPSQDPKVIALGTGTKCVSGSLLSPRGDIVNDSHAEVVARRALIRFFYSEIQRMQLTSGKSNEAKRQRIDSETSSILESADSSCPGEVKYKLKSGCLLHLYISQLPCGYASTSSPLYALKKIPSTQVDDSLLVQASDICSSRHSDVPEIGSNSNKGNGSQVADMVQRKPGRGETTLSVSCSDKIARWNVLGVQGALLYQVLQPVYISTITVGQSLHSPDNFSLADHLRRSLYERILPLSDELLTSFRLNKPLFFVAPVPPSEFQHSETAQATLTCGYSLCWNYSGLHEVILGTTGRKQGTSAKGALYPSTQSSICKQRLLELFLKETHGHKRESSKSKKSYRELKNKATEYYLMSKIFKGKYPFNNWLRKPLNCEDFLIN

SEQ ID NO: 849 - protein, 388 aa, unidentified
  source 1..388: note = Schizosaccharomyces pombe
MEEFTLDRNSNVGNLIALAVLNKFDELARHGKPIIRANGVREWTTLAGVVIQKKMENEFICVCLATGVKCTPAGIIKNEQLGSVLHDCHAEILALRCFNRLLLEHCILIKESKKDTWLLEVADNGKFTLNSNLLIHLYVSECPCGDASMELLASRLENNKPWNLTVDSEKLMRGRADFGLLGIVRTKPGRPDAPVSWSKSCTDKLAAKQYLSILNSQTSLICEPIYLSCVVLYKKVIVKSAIDRAFGPFGRCAPLAEFGEKDNPYYFHPFTVLETDENFLYSRPLNQAEKTATSTNVLIWIGDKMQCTQVIHNGIKAGTKAKDVEKSQTLICRKSMMNLLHQLSQSLTNEKNYYEWKKLNIKRCQQKQILRNILKNWIPNGGNEFQWI

SEQ ID NO: 850 - protein, 400 aa, Saccharomyces cerevisiae
MVSCQGTRPCIVNLLTMPSEDKLGEEISTRVINEYSKLKSACRPIIRPSGIREWTILAGVAAINRDGGANKIEILSIATGVKALPDSELQRSEGKILHDCHAEILALRGANTVLLNRIQNYNPSSGDKFIQHNDEIPARFNLKENWELALYISRLPCGDASMSFLNDNCKNDDFIKIEDSDEFQYVDRSVKTILRGRLNFNRRNVVRTKPGRYDSNITLSKSCSDKLLMKQRSSVLNCLNYELFEKPVFLKYIVIPNLEDETKHHLEQSFHTRLPNLDNEIKFLNCLKPFYDDKLDEEDVPGLMCSVKLFMDDFSTEEAILNGVRNGFYTKSSKPLRKHCQSQVSRFAQWELFKKIRPEYEGISYLEFKSRQKKRSQLIIAIKNILSPDGWIPTRTDDVK

SEQ ID NO: 851 - protein, 502 aa, Macaca fascicularis
MWTADEIALLCYEHYGIRLPKKGKPEPNHEWTLLAAVVKIQSPADQDCDTPDKPAQVTKEVVSMGTGTKCIGQSKMRKSGDILNDSHAEVIARRNFQRYLLHQLQLAATLKEDSIFVPGTQKGLWKLRRDLFFVFFSSHTPCGDASIIPMLEFEDQPCCPVIRDWASSSSVEASSNLEAPGNERKCEDLDSPVTKKMRLEPMTAAREVTNGATHHQSFGKQESGPISPGINSCNLTVEGLAAVTRIAPGSAKVIDVYRTGAKCVPGEAGDSRKPGAAFHQVGLLRVKPGRGDRTRSMSCSDKMARWNVLGCQGALLMHFLEEPIYLSAVVIGKCPYSQEAMQRALTGRRQNVSALPKGFGVQELKILQSDLLFEQSRCAVQAKRADSPGRLVPCGAAISWSAVPEQPLDVTANGFPQGTTKKTIGSLQARSQISKVELLRSFQKLLSRIARDKWPDSLRVQKLDTYQDYKEAASSYQEAWSTLRKQAFGSWIRNPPDYHQFK

SEQ ID NO: 852 - protein, 503 aa, Gallus gallus
MWTADEIAELCYEHYRSRLPKQGKPDPSREWTSLAAVVKVESAANEAGSAVLGTLQVAKEVVALGTGTKCIGLNKMRKTGDVLNDSHAEVVAKRSFQRYLLHQMRLATSYQQCSIFIPGTETGKWKLKPNIIFIFFCSHTPCGDASIIPIRETENHLSKSVDGHDIAGQSVLCSSSNCDHRGPEDKRKSEKMASSHMIKRMKNADGGFFSTITEDMAVQQVFAKPEGNVNPECCESSEEMQAANKETNAGKLKAVGVYRTGAKFVPGELSDTLIPGIEYHCVGLLRVKPGRGDRTCSMSCSDKLARWNVLGCQGALLMHFLQYPVYLSAVIVGKCPYSQEAMQRAVIERCRHISLLPDGFLTQEVQLLQSDLQFEHSRQAIQEGQTSSKRKLVPCSAAISWSAVPEGPLDVTSDGFRQGTTKKGIGSPQSRSKICKVELFHEFQKLVTSISKENLPDTLRMKTLETYWDYKEAALNYQEAWKALRSQALLGWIKNAQEYLLFM

SEQ ID NO: 853 - protein, 502 aa, Homo sapiens
MWTADEIAQLCYEHYGIRLPKKGKPEPNHEWTLLAAVVKIQSPADKACDTPDKPVQVTKEVVSMGTGTKCIGQSKMRKNGDILNDSHAEVIARRSFQRYLLHQLQLAATLKEDSIFVPGTQKGVWKLRRDLIFVFFSSHTPCGDASIIPMLEFEDQPCCPVFRNWAHNSSVEASSNLEAPGNERKCEDPDSPVTKKMRLEPGTAAREVTNGAAHHQSFGKQKSGPISPGIHSCDLTVEGLATVTRIAPGSAKVIDVYRTGAKCVPGEAGDSGKPGAAFHQVGLLRVKPGRGDRTRSMSCSDKMARWNVLGCQGALLMHLLEEPIYLSAVVIGKCPYSQEAMQRALIGRCQNVSALPKGFGVQELKILQSDLLFEQSRSAVQAKRADSPGRLVPCGAAISWSAVPEQPLDVTANGFPQGTTKKTIGSLQARSQISKVELFRSFQKLLSRIARDKWPHSLRVQKLDTYQEYKEAASSYQEAWSTLRKQVFGSWIRNPPDYHQFK

SEQ ID NO: 854 - protein, 499 aa, Mus musculus
MWTADEIAQLCYAHYNVRLPKQGKPEPNREWTLLAAVVKIQASANQACDIPEKEVQVTKEVVSMGTGTKCIGQSKMRESGDILNDSHAEIIARRSFQRYLLHQLHLAAVLKEDSIFVPGTQRGLWRLRPDLSFVFFSSHTPCGDASIIPMLEFEEQPCCPVIRSWANNSPVQETENLEDSKDKRNCEDPASPVAKKMRLGTPARSLSNCVAHHGTQESGPVKPDVSSSDLTKEEPDAANGIASGSFRVVDVYRTGAKCVPGETGDLREPGAAYHQVGLLRVKPGRGDRTCSMSCSDKMARWNVLGCQGALLMHFLEKPIYLSAVVIGKCPYSQEAMRRALTGRCEETLVLPRGFGVQELEIQQSGLLFEQSRCAVHRKRGDSPGRLVPCGAAISWSAVPQQPLDVTANGFPQGTTKKEIGSPRARSRISKVELFRSFQKLLSSIADDEQPDSIRVTKKLDTYQEYKDAASAYQEAWGALRRIQPFASWIRNPPDYHQFK

SEQ ID NO: 855 - protein, 394 aa, Drosophila melanogaster
MCDNKKPTVKEIAELCLKKFESLPKTGKPTANQWTILAGIVEFNRNTEACQLVSLGCGTKCIGESKLCPNGLILNDSHAEVLARRGFLRFLYQELKQDRIFHWNSTLSTYDMDEHVEFHFLSTQTPCGDACILEEEQPAARAKRQRLDEDSEMVYTGAKLISDLSDDPMLQTPGALRTKPGRGERTLSMSCSDKIARWNVIGVQGALLDVLISKPIYFSSLNFCCDDAQLESLERAIFKRFDCRTFKHTRFQPQRPQINIDPGIRFEFSQRSDWQPSPNGLIWSQVPEELRPYEISVNGKRQGVTKKKMKTSQAALAISKYKLFLTFLELVKFNPKLSEMFDQQLSDPERIAYASCKDLARDYQFAWREIKEKYFLQWTKKPHELLDFNPMSNK

SEQ ID NO: 856 - protein, 472 aa, Xenopus tropicalis
MQAKGLWSADEIAALSYGHYTTQLPKQGLPDPSREWTLMAAVIQIESVEDTKVIKKVVAMGTGTKCIGQAKLRKTGDVLQDSHAEIIAKRSFQRYLLHQLSLAVSDTKDCLFIPGTEKGKWMLRPEISFVFFTSHTPCGDASIIPVISHEDELGHPLPSEVTEKDHSSNNVCESVNTTYKRKVRSEEDIGFISKKMKHSIDEILTRPENYEEENRHDFPSTCQKALDVHRTGAKCVAGELQDSYSPGVNYHTVGVLRIKPGRGDRTMSMSCSDKMARWNVLGCQGALLMHFLQQPIYLSAVVVGKCPFSQDAMERALYNRCHKVLSLPCAFRLNRVQIIQSDLEFQHGRHALTKKDATRKLVPCGAAVSWSAVPHHPLDVTANGYRQGTTRKAIGSPQCRSRICKAEIFNTFRELVQRLSEKQRSESLSSQGLKTYWDYKAAAITYQEAWNCLRQQAFTSWIQTPRDFLMFS

SEQ ID NO: 857 - protein, 545 aa, unidentified
  source 1..545: note = Dictyostelium discoideum
MSWKLDKQFSDKICNFSHDFFNKKLIKKGKPISGEWTVLATLVLVVENTSSYEIKQVLSLGTGNRCLGKSSLSNQGDVLNDSHAEIICKRSFQKFCYNEILNLLQSKYYNSILFNIEYHDSNNNNKDNDNNGSLPTISIKKGHSLHFYVNQTPCGDCSIFPFKKETQPENFIEKEKLEKDGKDKIENHEKKEQKDIIKQVDKDKDEENYEDEESKRKLKKVKDDNNNNNNNNNNNNNNNNNNNNNNNNNNNNNNNNNNNNNINNNNNQYDDIQRTGAKTVFGEPEDKKLIGVDYHQIGVLRVKPGRGDPTVSMSCSDKIARWNVLGIQGSLLSHFIKEQIFLSSITIGDLFNHSSIYRGLIGRLLPNPTTTTTETSSSSSSSSSSSNTIPNFKLNSDLEIFSTNIQFQFSKLLLESDQQNNNKSTSSGLAISFCYPNQHEVTIAINGKKMGTNQKNFNAISQRSSICKFNLFKLFHQLVLIIKNKNSNEENEKNNQIVLIDSLFNYYECKHLSKKYYQEYEKLKEFKFKNWLTNSSDLENFVLDN

SEQ ID NO: 858 - protein, 389 aa, unidentified
  source 1..389: note = Schizosaccharomyces pombe
MAGDSVKSAIIGIAGGPFSGKTQLCEQLLERLKSSAPSTFSKLIHLTSFLYPNSVDRYALSSYDIEAFKKVLSLISQGAEKICLPDGSCIKLPVDQNRIILIEGYYLLLPELLPYYTSKIFVYEDADTRLERCVLQRVKAEKGDLTKVLNDFVTLSKPAYDSSIHPTRENADIILPQKENIDTALLFVSQHLQDILAEMNKTSSSNTVKYDTQHETYMKLAHEILNLGPYFVIQPRSPGSCVFVYKGEVIGRGFNETNCSLSGIRHAELIAIEKILEHYPASVFKETTLYVTVEPCLMCAAALKQLHIKAVYFGCGNDRFGGCGSVFSINKDQSIDPSYPVYPGLFYSEAVMLMREFYVQENVKAPVPQSKKQRVLKREVKSLDLSRFK

SEQ ID NO: 859 - protein, 250 aa, Saccharomyces cerevisiae
MQHIKHMRTAVRLARYALDHDETPVACIFVHTPTGQVMAYGMNDTNKSLTGVAHAEFMGIDQIKAMLGSRGVVDVFKDITLYVTVEPCIMCASALKQLDIGKVVFGCGNERFGGNGTVLSVNHDTCTLVPKNNSAAGYESIPGILRKEAIMLLRYFYVRQNERAPKPRSKSDRVLDKNTFPPMEWSKYLNEEAFIETFGDDYRTCFANKVDLSSNSVDWDLIDSHQDNIIQELEEQCKMFKFNVHKKSKV

SEQ ID NO: 860 - protein, 170 aa, Xenopus tropicalis
MTEEIQNWMHKAFQMAQDALNNGEVPVGCLMVYDNQVVGKGRNEVNETKNATRHAEMVAIDQVLDWCEKNSKKSRDVFENIVLYVTVEPCIMCAGALRLLKIPLVVYGCRNERFGGCGSVLNVAGDNIPDTGTEFKYIGGYQAEKAVELLKTFYKQENPNAPRSKVRKKE

SEQ ID NO: 861 - protein, 191 aa, Bos taurus
MEAKAGPTAATDGAYSVSAEETEKWMEQAMQMAKDALDNTEVPVGCLMVYNNEVVGKGRNEVNQTKNATRHAEMVAIDQALDWCRRRGRSPSEVFEHTVLYVTVEPCIMCAAALRLMRIPLVVYGCQNERFGGCGSVLDIASADLPSTGKPFQCTPGYRAEEAVEMLKTFYKQENPNAPKSKVRKKECHKS

SEQ ID NO: 862 - protein, 214 aa, Danio rerio
MQEVGVDPEKNDFLQPSDSEVQTWMAKAFDMAVEALENGEVPVGCLMVYNNEIIGKGRNEVNETKNATRHAEMVALDQVLDWCRLREKDCKEVCEQTVLYVTVEPCIMCAAALRLLRIPFVVYGCKNERFGGCGSVLDVSSDHLPHTGTSFKCIAGYRAEEAVEMLKTFYKQENPNAPKPKVRKDSINPQDGAAVIQVMRGPPDEETETIAHLS

SEQ ID NO: 863 - protein, 182 aa, Arabidopsis thaliana
MEEDHCEDSHNYMGFALHQAKLALEALEVPVGCVFLEDGKVIASGRNRTNETRNATRHAEMEAIDQLVGQWQKDGLSPSQVAEKFSKCVLYVTCEPCIMCASALSFLGIKEVYYGCPNDKFGGCGSILSLHLGSEEAQRGKGYKCRGGIMAEEAVSLFKCFYEQGNPNAPKPHRPVVQRERT

SEQ ID NO: 864 - protein, 191 aa, Mus musculus
MEEKVESTTTPDGPCVVSVQETEKWMEEAMRMAKEALENIEVPVGCLMVYNNEVVGKGRNEVNQTKNATRHAEMVAIDQVLDWCHQHGQSPSTVFEHTVLYVTVEPCIMCAAALRLMKIPLVVYGCQNERFGGCGSVLNIASADLPNTGRPFQCIPGYRAEEAVELLKTFYKQENPNAPKSKVRKKDCQKS

SEQ ID NO: 865 - protein, 191 aa, Homo sapiens
MEAKAAPKPAASGACSVSAEETEKWMEEAMHMAKEALENTEVPVGCLMVYNNEVVGKGRNEVNQTKNATRHAEMVAIDQVLDWCRQSGKSPSEVFEHTVLYVTVEPCIMCAAALRLMKIPLVVYGCQNERFGGCGSVLNIASADLPNTGRPFQCIPGYRAEEAVEMLKTFYKQENPNAPKSKVRKKECQKS

SEQ ID NO: 866 - protein, 400 aa, Arabidopsis thaliana
MDSDAWEIIHIPEKPSLSPDHQPTVKVYASLIKPRFANTIVRHLCKIAPLEDLRHVKRVKKKILPDCGETQLTVILCLAPEHNDQLSDMPPDVQRLVDPYELSPFITQVCKYAAVSKEEWEEQSKIWPTSFHPPTYNIDGIGGFSEEETQSICKFMRVVIDMAVSGHTPLVNAAVIVDPSVRRIIASETDQVYASSAPRDMTSAETRPFEETGEICLNDTLEKQNGSLSALSCLNPWQWSLQPHDTENCSQWHPLRHASMVAIESSSARDRNLFPNPSKIFDQDHVPPSNTDSPAKKQKTSSQSPDVQNDSREETVRDPSMERPYLCTGYDIFLLLEPCTMCAMALVHQRIKRIFYAFPNTTAGGLGSVHRLQGEKSLNHHYAVFRVLLPDDALRQMTTV

SEQ ID NO: 867 - protein, 349 aa, Mus musculus
MEPTSGFAEQPGPVKAESEEQEPAQWQALPVLSEQQSGAVELILAYAAPVLDKRQTSRLLREVSAVYPLPAQPHLKRVRPSRSAGGAQSSDLLLCLAGPSAGPRSLAELLPRPAVDPRGLGTPFLVPVPARPPLTRSQFEEARAHWPTSFHEDKQVTSALAGQLFSTQERAAMQTHMERAVCAAQRAAAQGLRAVGAVVVDPASDRVLATGHDCSSVASPLLHAVMVCIDLVAQGQGRGSCDLRSHPACSFTQATATQGARAGSVRKLDEDSLPYVCTGYDLYVTREPCVMCAMALVHARIQRVFYGAPSPDGALGTLFRVHARPDLNHRFQVFRGILEDQCRQLDPDP

SEQ ID NO: 868 - protein, 336 aa, Danio rerio
MEPQAKRKKEMDDYDDTWEVLPVLSDEQSQDPELLPAYAAPILERRETSRLVKELSLIHPLPNLQHIKRVRPCKHKDSPHPLEVIVCLVSDVQCTDPKKVTLSHLLHTQCFNSNGLGDPFIVQIPANPPLTRPQFEKASKHWPTSFHEDKLVTFALKGQLFTAHQKTKMREYMCVAVKAAKSGRELGMDAVGAVIVDPKTEQIIAVAHDCKRGSHPLHHAVMVCIDLVACGQDGGAYNYEKYPACRFSCSNSVCDGKETGLPYICTGYDLYVTREPCVMCAMALVHSRISRVFYGASTADGAFGSRYKIHCQKDLNHRFEVFKGVMVNACEDLCKE

SEQ ID NO: 869 - protein, 315 aa, unidentified
  source 1..315: note = Schizosaccharomyces pombe
MVKTNISKNSPKEATVPELDWPFKLIKSHLETRKLETENVWIACFEPKYASKVTQYVKQIRSKQKESLLHCNRLRRIQDENGSLELQIIICPEKSMTANEIGKDFEDLGIVSKMIFLYAVPAFPPLTDEQFHEWNSVWPVSYRKHVQRQDVFTVHELKRIESILEDLINAAGASHKHGEIGCAAAIYDPTTDTVLAVSVDERSKLKNPINHCVMNAINLVAKRELSRRQNRTDGSKDRYLCKDLTVVMTHEPCVMCSMGLLHSRIRRLIYCKKQPLTGGIESLYGIHWRAELNHRYLAYSGWNKPVPSIKENIHV

SEQ ID NO: 870 - protein, 322 aa, Saccharomyces cerevisiae
MVKKVNNPLKIDYQNGIIENRLLQIRNFKDVNTPKLINVWSIRIDPRDSKKVIELIRNDFQKNDPVSLRHLKRIRKDIETSTLEVVLCSKEYICDEGEINNKLKSIWVGTKKYELSDDIEVPEFAPSTKELNNAWSVKYWPLIWNGNPNDQILNDYKIDMQEVRNELSRASTLSVKMATAGKQFPMVSVFVDPSRKKDKVVAEDGRNCENSLPIDHSVMVGIRAVGERLREGVDEDANSYLCLDYDVYLTHEPCSMCSMALIHSRVRRVVFLTEMQRTGSLKLTSGDGYCMNDNKQLNSTYEAFQWIGEEYPVGQVDRDVCC

SEQ ID NO: 871 - protein, 351 aa, Homo sapiens
MEPAPGLVEQPKCLEAGSPEPEPAPWQALPVLSEKQSGDVELVLAYAAPVLDKRQTSRLLKEVSALHPLPAQPHLKRVRPSRDAGSPHALEMLLCLAGPASGPRSLAELLPRPAVDPRGLGQPFLVPVPARPPLTRGQFEEARAHWPTSFHEDKQVTSALAGRLFSTQERAAMQSHMERAVWAARRAAARGLRAVGAVVVDPASDRVLATGHDCSCADNPLLHAVMVCVDLVARGQGRGTYDFRPFPACSFAPAAAPQAVRAGAVRKLDADEDGLPYLCTGYDLYVTREPCAMCAMALVHARILRVFYGAPSPDGALGTRFRIHARPDLNHRFQVFRGVLEEQCRWLDPDT

SEQ ID NO: 872 - protein, 349 aa, Rattus norvegicus
MEPTSGFAEQPGPEKVESEEQEPAQWQALPVLSEQQSGAVELVLAYAAPVLDKRQTSRLLREVSAVYPLPAQPHLKRVRPSRSAGGAHSSDLLLCLAGPSAGPRSLAELLPRPAVDPRGLGTPFLVPVPARPPLTRSQFEEARAHWPTSFHEDKQVTSALAGQLFSAQARAAMQTHMERAVRAAQRAAAQGLRAVGAVVVDPASDHVLATGHDCCSEASPLLHAVMVCIDLVAQGQGRGSCDLRRHPACSFTQATATQSARAGSVRKLDEDSLPYVCTGYDLYVTREPCVMCAMALVHARIQRVFYGAPSPDGALGTRFRVHARPDLNHRFQVFRGILEDQCRQLDPDP

SEQ ID NO: 873 - protein, 711 aa, Rattus norvegicus
MDIEDEENMSSSSIDVKENRNLDNMPPKDSSTPGPGEGIPLSNGGGGSTSRKRPLEEGSNGHSKYRLKKRRKTPGPVLPKNALMQLNEIKPGLQYMLLSQTGPVHAPLFVMSVEVNGQVFEGSGPTKKKAKLHAAEKALRSFVQFPNASEAHLAMGRTLSVNTDFTSDQADFPDTLFNGFETPDKSEPPFYVGSNGDDSFSSSGDVSLSASPVPASLTQPPLPIPPPFPPPSGKNPVMILNELRPGLKYDFLSESGESHAKSFVMSVVVDGQFFEGSGRNKKLAKARAAQSALATVFNLHLDQTPSRQPVLSEGLQLHLPQVLADAVSRLVLGKFSDLTDNFSSPHARRKVLSGVVMTTGTDVKDAKVISVSTGTKCINGEYMSDRGLALNDCHAEIISRRSLLRFLYAQLELYLNNKEDQKKSIFQKSERGGFRLKDTVQFHLYISTSPCGDARIFSPHEPVLEGMAPDSHQLTEPADRHPNRKARGQLRTKIESGEGTIPVRSNASIQTWDGVLQGERLLTMSCSDKIARWNVVGIQGALLSIFVEPIYFSSIILGSLYHGDHLSRAMYQRISNIEDLPPLYTLNKPLLSGISNAEARQPGKAPNFSVNWTVGDTAIEVINATTGKDELGRPSRLCKHALYCRWMRVHGKVPPHLLRTKITKPTTYHESKLAAKEYQAAKARLFTAFIKAGLGAWVEKPTEQDQFSFTP

SEQ ID NO: 874 - protein, 1226 aa, Homo sapiens
MNPRQGYSLSGYYTHPFQGYEHRQLRYQQPGPGSSPSSFLLKQIEFLKGQLPEAPVIGKQTPSLPPSLPGLRPRFPVLLASSTRGRQVDIRGVPRGVHLRSQGLQRGFQHPSPRGRSLPQRGVDCLSSHFQELSIYQDQEQRILKFLEELGEGKATTAHDLSGKLGTPKKEINRVLYSLAKKGKLQKEAGTPPLWKIAVSTQAWNQHSGVVRPDGHSQGAPNSDPSLEPEDRNSTSVSEDLLEPFIAVSAQAWNQHSGVVRPDSHSQGSPNSDPGLEPEDSNSTSALEDPLEFLDMAEIKEKICDYLFNVSDSSALNLAKNIGLTKARDINAVLIDMERQGDVYRQGTTPPIWHLTDKKRERMQIKRNTNSVPETAPAAIPETKRNAEFLTCNIPTSNASNNMVTTEKVENGQEPVIKLENRQEARPEPARLKPPVHYNGPSKAGYVDFENGQWATDDIPDDLNSIRAAPGEFRAIMEMPSFYSHGLPRCSPYKKLTECQLKNPISGLLEYAQFASQTCEFNMIEQSGPPHEPRFKFQVVINGREFPPAEAGSKKVAKQDAAMKAMTILLEEAKAKDSGKSEESSHYSTEKESEKTAESQTPTPSATSFFSGKSPVTTLLECMHKLGNSCEFRLLSKEGPAHEPKFQYCVAVGAQTFPSVSAPSKKVAKQMAAEEAMKALHGEATNSMASDNQPEGMISESLDNLESMMPNKVRKIGELVRYLNTNPVGGLLEYARSHGFAAEFKLVDQSGPPHEPKFVYQAKVGGRWFPAVCAHSKKQGKQEAADAALRVLIGENEKAERMGFTEVTPVTGASLRRTMLLLSRSPEAQPKTLPLTGSTFHDQIAMLSHRCFNTLTNSFQPSLLGRKILAAIIMKKDSEDMGVVVSLGTGNRCVKGDSLSLKGETVNDCHAEIISRRGFIRFLYSELMKYNSQTAKDSIFEPAKGGEKLQIKKTVSFHLYISTAPCGDGALFDKSCSDRAMESTESRHYPVFENPKQGKLRTKVENGEGTIPVESSDIVPTWDGIRLGERLRTMSCSDKILRWNVLGLQGALLTHFLQPIYLKSVTLGYLFSQGHLTRAICCRVTRDGSAFEDGLRHPFIVNHPKVGRVSIYDSKRQSGKTKETSVNWCLADGYDLEILDGTRGTVDGPRNELSRVSKKNIFLLFKKLCSFRYRRDLLRLSYGEAKKAARDYETAKNYFKKGLKDMGYGNWISKPQEEKNFYLCPV

SEQ ID NO: 875 - protein, 1175 aa, Rattus norvegicus
MSQGFRGPTGVFPHQTQPCLDPSYEHSKWRYLQPRGSESYLRSFQLQQIEFLKGRLPEAPLIGAQTQSLPPFLPGHWPRFPGPPAQGKQPEIWGFPRSVTLRNQGFHIGPPLPPPHSRGPPWRGAEGLCSHFQELSISQNPEQKVLNRLEELGEGKATTAYALARELRTPKKDINRILYSLERKGKLHRGVGKPPLWSLVPLSQACTQPPRAVNSDKEVPRGEPDLDSEDGDPASDLEGPSELLDMAEIKEKICDYLFNVSKSSALNLAKNIGLAKARDVNAVLIDLERQGDVYREGATPPIWYLTDKKRERLQMKRSTHSGPAATPAAVSEATQSTSFPTCHPPQSGGSSSMATSKRVENGQEPVTKYESRHEARPGPVRLRPHAYHNGPSRAGYVASENGPWATDDIPDNLNSIHTAPGEFRAIMEMPSFYSPTLPRCSPYKKLTECQLKNPVSGLLEYAQFTSQTCDFNLIEQSGPSHEPRFKFQVVINGREFPPAEAGSKKVAKQDAAVKAMAILLREAKAKDSGQPEELSNCPMEEDPEKPAESQPPSSSATSLFSGKSPVTTLLECMHKLGNSCEFRLLSKEGPAHDPKFQYCVAVGAQTFPSVSAPSKKVAKQMAAEEAMKALQEEAANSADDQSGGANTDSLDESVAPNKIRRIGELVRYLNTNPVGGLLEYARSHGFAAEFKLIDQSGPPHEPKFVYQAKVGGRWFPAVCAHSKKQGKQDAADAALRVLIGESEKAEQLGFAEVTPVTGASLRRTMLLLSRSPDAHPKTLPLTGSTFHDQIAMLSHRCFNALTNSFQPSLLGRKILAAIIMKRDPEDMGVVVSLGTGNRCVKGDSLSLKGETVNDCHAEIISRRGFIRFLYSELMKYNHHTAKNSIFELARGGEKLQIKKTVSFHLYISTAPCGDGAHFDKSCSDRAVESTESRHYPVFENPKQGKLRTKVENGEGTIPVESSDIVPTWDGIRLGERLRTMSCSDKILRWNVLGLQGALLTHFLQPVYLKSVTLGYLFSQGHLTRAICCRVTRDGNAFEDGLRYPFIVNHPKVGRVSVYDSKRQSGKTKETSVNWCLADGYDLEILDGTRGTVDGPGKELSRVSKKNIFLQFKKLCSFRARRDLLQLSYGEAKKAARDYDLAKNYFKKSLRDMGYGNWISKPQEEKNFYLCPVPND

SEQ ID NO: 876 - protein, 741 aa, Homo sapiens
MDIEDEENMSSSSTDVKENRNLDNVSPKDGSTPGPGEGSQLSNGGGGGPGRKRPLEEGSNGHSKYRLKKRRKTPGPVLPKNALMQLNEIKPGLQYTLLSQTGPVHAPLFVMSVEVNGQVFEGSGPTKKKAKLHAAEKALRSFVQFPNASEAHLAMGRTLSVNTDFTSDQADFPDTLFNGFETPDKAEPPFYVGSNGDDSFSSSGDLSLSASPVPASLAQPPLPVLPPFPPPSGKNPVMILNELRPGLKYDFLSESGESHAKSFVMSVVVDGQFFEGSGRNKKLAKARAAQSALAAIFNLHLDQTPSRQPIPSEGLQLHLPQVLADAVSRLVLGKFGDLTDNFSSPHARRKVLAGVVMTTGTDVKDAKVISVSTGTKCINGEYMSDRGLALNDCHAEIISRRSLLRFLYTQLELYLNNKDDQKRSIFQKSERGGFRLKENVQFHLYISTSPCGDARIFSPHEPILEGSRSYTQAGVQWCNHGSLQPRPPGLLSDPSTSTFQGAGTTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDSAIEVINATTGKDELGRASRLCKHALYCRWMRVHGKVPSHLLRSKITKPNVYHESKLAAKEYQAAKARLFTAFIKAGLGAWVEKPTEQDQFSLTP

SEQ ID NO: 877 - protein, 711 aa, Mus musculus
MDIEDEENMSSSSTDIKENRNLDNMPPKDSSTPGPGEGIPLSNGGGGSTSRKRPLEEGSNGHSKYRLKKRRKTPGPVLPKNALMQLNEIKPGLQYMLLSQTGPVHAPLFVMSVEVNGQVFEGSGPTKKKAKLHAAEKALRSFVQFPNASEAHLAMGRTLSVNTDFTSDQADFPDTLFNGFETPDKSEPPFYVGSNGDDSFSSSGDVSLSASPVPASLTQPPLPIPPPFPPPSGKNPVMILNELRPGLKYDFLSESGESHAKSFVMSVVVDGQFFEGSGRNKKLAKARAAQSALATVFNLHLDQTPSRQPVLSEGLQLHLPQVLADAVSRLVLGKFSDLTDNFSSPHARRKVLSGVVMTTGTDVKDAKVISVSTGTKCINGEYMSDRGLALNDCHAEIISRRSLLRFLYAQLELYLNNKEDQKKSIFQKSERGGFRLKDTVQFHLYISTSPCGDARIFSPHEPVLEGMTPDSHQLTEPADRHPNRKARGQLRTKIESGEGTIPVRSNASIQTWDGVLQGERLLTMSCSDKIARWNVVGIQGSLLSIFVEPIYFSSIILGSLYHGDHLSRAMYQRISNIEDLPPLYTLNKPLLSGISNAEARQPGKAPNFSVNWTVGDATIEVINATTGKDELGRPSRLCKHALYCRWMRVHGKVPPHLLRTKITKPTTYHESKLAAREYQAAKARLFTAFIKAGLGAWVEKPTEQDQFSFTP

SEQ ID NO: 878 - protein, 1178 aa, Mus musculus
MSQGFRGPTGVFPHQTQSYLDPSHEHSKWRYPQPQGPESYPRSFQLQQIEFLKGRLPEAPLIGIQTQSLPPFLPGHWPRFPGPPAQDRQLEIWEFPRSVTLRNQGFHIGPPLPPPHSRGTPWRGADGLCSHFRELSISQSPEQKVLNRLEELGEGKATTAHVLARELRIPKRDINRILYSLEKKGKLHRGRGKPPLWSLVPLSQAWTQPPGVVNPDSCIQEFPRGEPGLDSEDGDPASDLEGPSEPLDMAEIKEKICDYLFNVSNSSALNLAKNIGLTKARDVTSVLIDLERQGDVYRQGATPPIWYLTDKKRERLQMKRSTHSAPAPTPTAVPEATRSPSFPACHPPPAGASSSVAASKRVENGQEPAIKHESRHEARPGPMRLRPHAYHNGPSRAGYVASENGQWATDDIPDNLNSIHTAPGEFRAIMEMPSFYSPTLPRCSPYKKLTECQLKNPVSGLLEYAQFTSQTCDFNLIEQSGPSHEPRFKFQVVINGREFPPAEAGSKKVAKQDAAVKAMAILLREAKAKDSGQPEDLSHCPMEEDSEKPAEAQAPSSSATSLFSGKSPVTTLLECMHKLGNSCEFRLLSKEGPAHDPKFQYCVAVGAQTFPPVSAPSKKVAKQMAAEEAMKALQEEAASSADDQSGGANTDSLDESMAPNKIRRIGELVRYLNTNPVGGLLEYARSHGFAAEFKLIDQSGPPHEPKFVYQAKVGGRWFPAVCAHSKKQGKQDAADAALRVLIGESEKAEQLGFAEVTPVTGASLRRTMLLLSRSPDAHPKTLPLSGSTFHDQIAMLSHRCFNALTNSFQPSLLGRKILAAIIMKRDPEDMGVVVSLGTGNRCVKGDSLSLKGETVNDCHAEIISRRGFIRFLYSELMKYNHHTAKNSIFELARGGEKLQIKKTVSFHLYISTAPCGDGALFDKSCSDRAVESTESRHYPVFENPKQGKLRTKVENGEGTIPVESSDIVPTWDGIRLGERLRTMSCSDKILRWNVLGLQGALLTHFLQPVYLKSVTLGYLFSQGHLTRAICCRVTRDGKAFEDGLRYPFIVNHPKVGRVSVYDSKRQSGKTKETSVNWCMADGYDLEILDGTRGTVDGPGKELSRVSKKNIFLQFKKLCSFRARRDLLQLSYGEAKKAARDYDLAKNYFKKSLRDMGYGNWISKPQEEKNFYLCPVPND

SEQ ID NO: 879 - protein, 147 aa, Homo sapiens
MKQLFPPPPGTSLTHALGAWRGRERAQAATSLLASSASQFPTAVEDALMSVLTSHCAPSTPAATRAQQTGTRGHIHPACPCQQSCVGASRPPGRPQIFLPLTTALSLEAYAADTCSAADFLHNPSSWGKVWYLNEASFDLYSYHYFW

SEQ ID NO: 880 - protein, 746 aa, Rattus norvegicus
MASVLGSGRGSGGLSSQLKCKSKRRRRRRSKRKDKVSILSTFLAPFKYLSPGTTNTEDEDNLSTSSAEVKENRNVSNLGTRPLPPGDWARGGSTPSVKRKRPLEEGNGGHFCKLQLIWKKLSWSMTPKNALVQLHELKPGLQYRMVSQTGPVHAPVFAVAVEVNGLTFEGTGPTKKKAKMRAAEMALKSFVQFPNAFQAHLAMGSSTSPCTDFTSDQADFPDTLFKEFEPSSRNEDFPGCCPVDTEFLSSAYRRGRLLYHTLDLMGQALPDRSRLAPGALGERNPVVVLNELRSGLRYVCLSETAEKPRVKSFVMAVCVDGRTFEGSGRSKKLAKGQAAQAALQALFDIRLPGHIPSRSKSNLLPQDFADSVSQLVTQKFRELTVGLTSVYARHKTLAGIVMTKGLDTKQAQVIVLSSGTKCISGEHISDQGLVVNDCHAEIVARRAFLHFLYTQLELHLSKHQEDPERSIFIRVKEGGYRLRENILFHLYVSTSPCGDARLNSPYEITIDLNSSKHIVRKFRGHLRTKIESGEGTVPVRGPSAVQTWDGILLGEQLVTMSCTDKIASWNVLGLQGALLCHFIEPVYLHSIIVGSLHHTGHLARVMSHRMEGIGQLPASYRQNRPLLSGVSNAEARQPGKSPHFSANWVVGSADLEIINATTGKRSCGGSSRLCKHVFSARWARLHGRLSTRIPGHGDTPSMYCEAKRGAHTYQSVKQQLFKAFQKAGLGTWVRKPPEQDQFLLSL

SEQ ID NO: 881 - protein, 745 aa, Mus musculus
MASVLGSGRGSGGLSSQLKCKSKRRRRRRSKRKDKVSILSTFLAPFKYLSPGTTNTEDEDNLSTSSAEVKENRNVSNLGTRPLPPGDWARGSTPSVKRKRPLEEGNGGHFCKLQLIWKKLSWSVTPKNALVQLHELKPGLQYRMVSQTGPVHAPVFAVAVEVNGLTFEGTGPTKKKAKMRAAEMALKSFVQFPNAFQAHLAMGSSTSPCTDFTSDQADFPDTLFKEFEPSSKNEDFPGCHPVDTEFLSSAYRRGRLLYHTLDLMGQALPDRSRLAPGALGERNPVVVLNELRSGLRYVCLSETAEKPRVKSFVMAVCVDGRTFEGSGRSKKLAKGQAAQAALQALFDIRLPGHIPSRSKSNLLPQDFADSVSQLVTQKFRELTVGLTSVYARHKTLAGIVMTKGLDTKQAQVIVLSSGTKCISGEHISDQGLVVNDCHAEIVARRAFLHFLYSQLELHLSKHQEDPERSIFIRLKEGGYRLRENILFHLYVSTSPCGDARVNSPYEITTDLNSSKHIVRKFRGHLRTKIESGEGTVPVRGPSAVQTWDGILLGEQLITMSCTDKIASWNVLGLQGALLCHFIEPVYLHSIIVGSLHHTGHLARVMSHRMEGIGQLPASYRQNRPLLSGVSHAEARQPGKSPHFSANWVVGSADLEIINATTGKRSCGGSSRLCKHVFSAWWARLHGRLSTRIPSHGDTPSMYCEAKQGAHTYQSVKQQLFKAFQKAGLGTWVRKPPEQDQFLLSL

SEQ ID NO: 882 - protein, 739 aa, Homo sapiens
MASVLGSGRGSGGLSSQLKCKSKRRRRRRSKRKDKVSILSTFLAPFKHLSPGITNTEDDDTLSTSSAEVKENRNVGNLAARPPPSGDRARGGAPGAKRKRPLEEGNGGHLCKLQLVWKKLSWSVAPKNALVQLHELRPGLQYRTVSQTGPVHAPVFAVAVEVNGLTFEGTGPTKKKAKMRAAELALRSFVQFPNACQAHLAMGGGPGPGTDFTSDQADFPDTLFQEFEPPAPRPGLAGGRPGDAALLSAAYGRRRLLCRALDLVGPTPATPAAPGERNPVVLLNRLRAGLRYVCLAEPAERRARSFVMAVSVDGRTFEGSGRSKKLARGQAAQAALQELFDIQMPGHAPGRARRTPMPQEFADSISQLVTQKFREVTTDLTPMHARHKALAGIVMTKGLDARQAQVVALSSGTKCISGEHLSDQGLVVNDCHAEVVARRAFLHFLYTQLELHLSKRREDSERSIFVRLKEGGYRLRENILFHLYVSTSPCGDARLHSPYEITTDLHSSKHLVRKFRGHLRTKIESGEGTVPVRGPSAVQTWDGVLLGEQLITMSCTDKIARWNVLGLQGALLSHFVEPVYLQSIVVGSLHHTGHLARVMSHRMEGVGQLPASYRHNRPLLSGVSDAEARQPGKSPPFSMNWVVGSADLEIINATTGRRSCGGPSRLCKHVLSARWARLYGRLSTRTPSPGDTPSMYCEAKLGAHTYQSVKQQLFKAFQKAGLGTWVRKPPEQQQFLLTL

SEQ ID NO: 883 - protein, 747 aa, Rattus norvegicus
MPLFKLTGQGKQIDDAMRSFAEKVFASEVKDEGGRHEISPFDVDEICPISLREMQAHIFHMENLSMSMDGRRKRRFQGRKTVNLSIPQSETSSTKLSHIEEFISSSPTYESVPDFQRVQITGDYASGVTVEDFEVVCKGLYRALCIREKYMQKSFQRFPKTPSKYLRNIDGEALVAIESFYPVFTPPPKKGEDPFRREDLPANLGYHLKMKGGVIYIYPDEAAASRDEPKPYPYPNLDDFLDDMNFLLALIAQGPVKTYTHRRLKFLSSKFQVHQMLNEMDELKELKNNPHRDFYNCRKVDTHIHAAACMNQKHLLRFIKKSYHIDADRVVYSTKEKNLTLKELFAQLNMHPYDLTVDSLDVHAGRQTFQRFDKFNDKYNPVGASELRDLYLKTDNYINGEYFATIIKEVGADLVDAKYQHAEPRLSIYGRSPDEWSKLSSWFVGNRIYCPNMTWMIQVPRIYDVFRSKNFLPHFGKMLENIFLPVFEATINPQTHPDLSVFLKHITGFDSVDDESKHSGHMFSSKSPKPEEWTMENNPSYTYYAYYMYANIMVLNCLRKERGMNTFLFRPHCGEAGALTHLMTAFMIADNISHGLNLKKSPVLQYLFFLAQIPIAMSPLSNNSLFLEYAKNPFLDFLQKGLMISLSTDDPMQFHFTKEPLMEEYAIAAQVFKLSTCDMCEVARNSVLQCGISHEEKAKFLGNNYLEEGPVGNDIRRTNVAQIRMAYRYETWCYELNLIAEGLKSTE

SEQ ID NO: 884 - protein, 747 aa, Homo sapiens
MPLFKLPAEEKQIDDAMRNFAEKVFASEVKDEGGRQEISPFDVDEICPISHHEMQAHIFHLETLSTSTEARRKKRFQGRKTVNLSIPLSETSSTKLSHIDEYISSSPTYQTVPDFQRVQITGDYASGVTVEDFEIVCKGLYRALCIREKYMQKSFQRFPKTPSKYLRNIDGEAWVANESFYPVFTPPVKKGEDPFRTDNLPENLGYHLKMKDGVVYVYPNEAAVSKDEPKPLPYPNLDTFLDDMNFLLALIAQGPVKTYTHRRLKFLSSKFQVHQMLNEMDELKELKNNPHRDFYNCRKVDTHIHAAACMNQKHLLRFIKKSYQIDADRVVYSTKEKNLTLKELFAKLKMHPYDLTVDSLDVHAGRQTFQRFDKFNDKYNPVGASELRDLYLKTDNYINGEYFATIIKEVGADLVEAKYQHAEPRLSIYGRSPDEWSKLSSWFVCNRIHCPNMTWMIQVPRIYDVFRSKNFLPHFGKMLENIFMPVFEATINPQADPELSVFLKHITGFDSVDDESKHSGHMFSSKSPKPQEWTLEKNPSYTYYAYYMYANIMVLNSLRKERGMNTFLFRPHCGEAGALTHLMTAFMIADDISHGLNLKKSPVLQYLFFLAQIPIAMSPLSNNSLFLEYAKNPFLDFLQKGLMISLSTDDPMQFHFTKEPLMEEYAIAAQVFKLSTCDMCEVARNSVLQCGISHEEKVKFLGDNYLEEGPAGNDIRRTNVAQIRMAYRYETWCYELNLIAEGLKSTE

SEQ ID NO: 885 - protein, 26 aa, Oryctolagus cuniculus
MNQKHLLRFIKKSYQVDADRVVYSTK

SEQ ID NO: 886 - protein, 26 aa, Gallus gallus
MNQKHLLRFIKKSYRVDADRVVYDAK

SEQ ID NO: 887 - protein, 745 aa, Mus musculus
MPLFKLTGQGKQIDDAMRSFAEKVFASEVKDEGGRHEISPFDVDEICPISLHEMQAHIFHMENLSMDGRRKRRFQGRKTVNLSIPQSETSSTKLSHIEEFISSSPTYESVPDFQRVQITGDYASGVTVEDFEVVCKGLYRALCIREKYMQKSFQRFPKTPSKYLRNIDGEALVGNESFYPVFTPPPKKGEDPFRTEDLPANLGYHLKMKAGVIYIYPDEAAANRDEPKPYPYPNLDDFLDDMNFLLALIAQGPVKTYAHRRLKFLSSKFQVHQMLNEMDELKELKNNPHRDFYNCRKVDTHIHAAACMNQKHLLRFIKKSYHIDADRVVYSTKEKSLTLKELFAKLNMHPYDLTVDSLDVHAGRQTFQRFDKFNDKYNPVGASELRDLYLKTDNYINGEYFATIIKEVGADLVEAKYQHAEPRLSIYGRSPDEWNKLSSWFVCNRIYCPNMTWMIQVPRIYDVFRSKNFLPHFGKMLENIFLPVFEATINPQAHPDLSVFLKHITGFDSVDDESKHSGHMFSSKSPKPEEWTMENNPSYTYYAYYMYANITVLNSLRKERGMNTFLFRPHCGEAGALTHLMTAFMIADNISHGLNLKKSPVLQYLFFLAQIPIAMSPLSNNSLFLEYAKNPFLDFLQKGLMISLSTDDPMQFHFTKEPLMEEYAIAAQVFKLSTCDMCEVARNSVLQCGISHEEKAKFLGNNYLEEGPVGNDIRRTNVAQIRMAYRYETWCYELNLIAEGLKATE

SEQ ID NO: 888 - protein, 790 aa, unidentified
  source 1..790: note = Dictyostelium discoideum
MSTPLRGSSPQVSFYESELDQEGGSDASHFTYRNYMEDDKINSFTFNMARKDQTQLFQRIILTNESESEIEEYAEVAEQLLDAINLREKYVFHPKIWKADAPVGEKPPYSPFESDESTNCATEHMFKEVNGVYFVYSNETDMKSNKALFSVPHTLASYYKDINNLMMLSSYGPAKTFTFKRLQLLESKFNMHTLLNDSLELFQQKTAPHRDFYNVRKVDTHVHHSSSMNQKHLLKFIKRKLKENPNEIVIFRDDKYLTLAEVFKSLNLDVDELSVDTLDVHADNNTFHRFDKFNLKYNPCGQSRLREIFLKTDNLIKGKYLAEISKEVFTDLESSKYQCAEYRLSIYGRKMSEWDTLASWIVDNDLFSTKVRWLIQVPRLYDVYRETSTTTFQDFLNNVFHPLFEVTKDPSSHPKLHLFLQQVVGIDCVDDESKFEKKFTEKFPVPGEWSSEHNPPYTYYLYYLYANLYTLNQFREEKGLNILTLRPHSGEAGEVDHMGAAFYLAHGINHGINLRKTPVLQYLYYLTQIGIAMSPLSNNSLFLTYNRNPFPAFFARGLNVSISTDDPLQFHYTKEPLMEEYSIATQVWRLSVCDICEIARNSVLQSGFEHNVKSHWLGPDYANSGGNDIKKTNISDIRVCFRNETLIEELHLILKSLQTLPNFKNLNINFLLDKLPSEITTGNDYKLKKAQLKLNGANKLRNSSVGSTPNNGTPSSSGTPSLSSPGAIVHLMKTKPYIPPPLSLNIKQENNNNNNNNNNNNNNNNNNNTNTNTNSNSTTTNQDDNSKSDK

SEQ ID NO: 889 - protein, 767 aa, Homo sapiens
MPRQFPKLNISEVDEQVRLLAEKVFAKVLREEDSKDALSLFTVPEDCPIGQKEAKERELQKELAEQKSVETAKRKKSFKMIRSQSLSLQMPPQQDWKGPPAASPAMSPTTPVVTGATSLPTPAPYAMPEFQRVTISGDYCAGITLEDYEQAAKSLAKALMIREKYARLAYHRFPRITSQYLGHPRADTAPPEEGLPDFHPPPLPQEDPYCLDDAPPNLDYLVHMQGGILFVYDNKKMLEHQEPHSLPYPDLETYTVDMSHILALITDGPTKTYCHRRLNFLESKFSLHEMLNEMSEFKELKSNPHRDFYNVRKVDTHIHAAACMNQKHLLRFIKHTYQTEPDRTVAEKRGRKITLRQVFDGLHMDPYDLTVDSLDVHAGRQTFHRFDKFNSKYNPVGASELRDLYLKTENYLGGEYFARMVKEVARELEESKYQYSEPRLSIYGRSPEEWPNLAYWFIQHKVYSPNMRWIIQVPRIYDIFRSKKLLPNFGKMLENIFLPLFKATINPQDHRELHLFLKYVTGFDSVDDESKHSDHMFSDKSPNPDVWTSEQNPPYSYYLYYMYANIMVLNNLRRERGLSTFLFRPHCGEAGSITHLVSAFLTADNISHGLLLKKSPVLQYLYYLAQIPIAMSPLSNNSLFLEYSKNPLREFLHKGLHVSLSTDDPMQFHYTKEALMEEYAIAAQVWKLSTCDLCEIARNSVLQSGLSHQEKQKFLGQNYYKEGPEGNDIRKTNVAQIRMAFRYETLCNELSFLSDAMKSEEITALTN

SEQ ID NO: 890 - protein, 879 aa, Homo sapiens
MRNRGQGLFRLRSRCFLHQSLPLGAGRRKGLDVAEPGPSRCRSDSPAVAAVVPAMASYPSGSGKPKAKYPFKKRASLQASTAAPEARGGLGAPPLQSARSLPGPAPCLKHFPLDLRTSMDGKCKEIAEELFTRSLAESELRSAPYEFPEESPIEQLEERRQRLERQISQDVKLEPDILLRAKQDFLKTDSDSDLQLYKEQGEGQGDRSLRERDVLEREFQRVTISGEEKCGVPFTDLLDAAKSVVRALFIREKYMALSLQSFCPTTRRYLQQLAEKPLETRTYEQGPDTPVSADAPVHPPALEQHPYEHCEPSTMPGDLGLGLRMVRGVVHVYTRREPDEHCSEVELPYPDLQEFVADVNVLMALIINGPIKSFCYRRLQYLSSKFQMHVLLNEMKELAAQKKVPHRDFYNIRKVDTHIHASSCMNQKHLLRFIKRAMKRHLEEIVHVEQGREQTLREVFESMNLTAYDLSVDTLDVHADRNTFHRFDKFNAKYNPIGESVLREIFIKTDNRVSGKYFAHIIKEVMSDLEESKYQNAELRLSIYGRSRDEWDKLARWAVMHRVHSPNVRWLVQVPRLFDVYRTKGQLANFQEMLENIFLPLFEATVHPASHPELHLFLEHVDGFDSVDDESKPENHVFNLESPLPEAWVEEDNPPYAYYLYYTFANMAMLNHLRRQRGFHTFVLRPHCGEAGPIHHLVSAFMLAENISHGLLLRKAPVLQYLYYLAQIGIAMSPLSNNSLFLSYHRNPLPEYLSRGLMVSLSTDDPLQFHFTKEPLMEEYSIATQVWKLSSCDMCELARNSVLMSGFSHKVKSHWLGPNYTKEGPEGNDIRRTNVPDIRVGYRYETLCQELALITQAVQSEMLETIPEEAGITMSPGPQ

SEQ ID NO: 891 - protein, 798 aa, Mus musculus
MASEARSGLGASPLQSARSLPGNAPCLKHFPLDLRTSMDGKCKEIAEELFSRSLAESELRSAPYEFPEESPIEQLEERRQRLERQISQDVKLEPDILLRAKQDFLKTDSDSDLQLYKEQGEGQGDRGLWERDVVLEREFQRVIISGEEKCGVPFTDLLDAAKSVVRALFIREKYMALSLQSFCPTTRRYLQQLAEKPLETRTYEQSPDTPVSADAPVHPPALEQHPYEHCEPSAMPGDLGLGLRMVRGVVHVYTRRDPDEHCPEVELPYPDLQEFVADVNVLMALIINGPIKSFCYRRLQYLSSKFQMHVLLNEMKELAAQKKVPHRDFYNIRKVDTHIHASSCMNQKHLLRFIKRAMKRHLEEIVHVEQGREQTLREVFESMNLTAYDLSVDTLDVHADRNTFHRFDKFNAKYNPIGESVLREIFIKTDNKISGKYFAHIIKEVMADLEESKYQNAELRLSIYGRSRDEWDKLARWAVNHKVHSPNVRWLVQVPRLFDVYRTKGQLANFQEMLENIFLPLFEATVHPASHPELHLFLEHVDGFDSVDDESKPENHVFNLESPLPEAWVEEDNPPYAYYLYYTFANMAMLNHLRRQRGFHTFVLRPHCGEAGPIHHLVSAFMLAENISHGLLLRKAPVLQYLYYLAQIGIAMSPLSNNSLFLSYHRNPLPEYLSRGLMVSLSTDDPLQFHFTKEPLMEEYSIATQVWKLSSCDMCELARNSVLMSGFSHKVKSHWLGPNYTKEGPEGNDIRRTNVPDIRVGYRYETLCQELALITQAVQSEMLETIPEEVGIVMSPGP

SEQ ID NO: 892 - protein, 824 aa, Rattus norvegicus
MASYPGPGKSKAKYPFKKRASLQASAAAPEARSGLGASPLQSARSLPGTAPCLKHFPLDLRTSMDGKCKEIAEELFSRSLAESELRSAPYEFPEESPIEQLEERRQRLERQISQDVKLEPDILLRAKQDFLKTDSDSDLQLYKEQGEGQGDRGLWERDVVLEREFQRVIISGEEKCGVPFTDLLDAAKSVVRALFIREKYMALSLQSFCPTTRRYLQQLAEKPLETRTYEQSPDTPVSADAPVHPPALEQHPYEHCEPSTMPGDLGLGLRMVRGVVHVYTRRDPDEHCPEVELPYPDLQEFVADVNVLMALIINGPIKSFCYRRLQYLSSKFQMHVLLNEMKELAAQKKVPHRDFYNIRKVDTHIHASSCMNQKHLLRFIKRAMKRHLEEIVHVEQGREQTLREVFESMNLTAYDLSVDTLDVHADRNTFHRFDKFNAKYNPIGESVLREIFIKTDNKISGKYFAHIIKEVMSDLEESKYQNAELRLSIYGRSRDEWDKLARWAVNHRVHSPNVRWLVQVPRLFDVYRTKGQLANFQEMLENIFLPLFEATVHPASHPELHLFLEHVDGFDSVDDESKPENHVFNLESPLPEAWVEEDNPPYAYYLYYTFANMAMLNHLRRQRGFHTFVLRPHCGEAGPIHHLVSAFMLAENISHGLLLRKAPVLQYLYYLAQIGIAMSPLSNNSLFLSYHRNPLPEYLSRGLMVSLSTDDPLQFHFTKEPLMEEYSIATQVWKLSSCDMCELARNSVLMSGFSHKVKSHWLGPNYTKEGPEGNDIRRTNVPDIRVGYRYETLCQELALITQAVQSEMLETIPEEVGIVMSPGP

SEQ ID NO: 893 - protein, 362 aa, unidentified
  source 1..362: note = Plasmodium gallinaceum
MTILHEEINFLKKDELNINLKCLDKKERYKIWRRIPKCELHCHLDLCFSLEFFLKCVRKYNLQPDLTDDEVVDYYLFKDKGKSLNEFIERSRRVTDIFINYDIIKDIAKNAVFNKYKEGVILIEFRYSPSYIAYKYNLCIDLIHKTIVEGINEAVEKLNHKIHVGLICIGETGISEESLRKAAEFCVKNKKDFVGFDHAGHERDLKPYKEIYDYVRENGIPLTIHAGEDLTLPNLNTIYSAIEVLKAKRIGHGIRVIESEDLINLIKKNDILLEICPISNLLLNNVKSMDTHPIKKLYDSGIKVSVNTDDPGMFLTEINDEYEELYLNLNFNLEDFMKMNLWALEKSFVKSEIKDKLKKLYF

SEQ ID NO: 894 - protein, 363 aa, unidentified
  source 1..363: note = Plasmodium knowlesi
MNILQEPIDFLKKDELKNIDLSQMDKKERYKIWKRIPKCELHCHLDLCFSADFFLSCVRKYNLQPNLSDEEVLDYYLFAKGGKSLGEFVEKAIRVADIFQDYEMIEDLAKHAVFNKYKEGVVLMEFRYSPTFVAFKHNLDIELIHQAIVKGIKEVVELLDHKIDVTLLCIGDTGHRAADIKASADFCLKHKADFVGFDHGGHEVDLKPYKEIFDYVKEGGMHLTVHAGEDVTLPNLNTLYSAIQVLKVERIGHGIRVSESQELIDMVKENNILLEVCPISNVLLKNAKSFDTHPIRKLYDAGVKVSVSSDDPGMFLTNINDDYEKLYTHLHFTLEDFMKMNEWALEKSFIGCDIKEKIKKLYF

SEQ ID NO: 895 - protein, 363 aa, unidentified
  source 1..363: note = Plasmodium berghei
MEIPNEEIKFLKKEDIKNINLNGMNKKERYEIWKKIPKVELHCHLDLTFSGKFFLKWVRKYNLQPNMTDDQVLDHYLFTKEGKSLAEFIRKAISVSDIYRDYDILEDLAKWAVIEKYKEGVVLMEFRYSPTFVSSSHGLDIELIHKAFVKGIKNATEMLNNKIYVALICISDTGHSAASIKHSGDFAIKHKHDFVGFDHGGREIDLKDHKDVYHSVRNHGLHLTVHAGEDATLPNLNTLYTAINILNVERIGHGIRVSESEELIELVKKNNILLEVCPISNLLLNNVKSMDTHPIRKLFDAGVKVSVNSDDPGMFLTDINDNYEKLYIHLNFTLEEFMTMNNWALEKSFVNDDIKSKLKTMYF

SEQ ID NO: 896 - protein, 363 aa, unidentified
  source 1..363: note = Plasmodium vivax
MNILQEPIDFLKKEELKNIDLSQMSKKERYKIWKRIPKCELHCHLDLCFSADFFVSCIRKYNLQPNLSDEEVLDYYLFAKGGKSLGEFVEKAIKVADIFHDYEVIEDLAKHAVFNKYKEGVVLMEFRYSPTFVAFKYNLDIELIHQAIVKGIKEVVELLDHKIHVALMCIGDTGHEAANIKASADFCLKHKADFVGFDHGGHEVDLKEYKEIFDYVRESGVPLSVHAGEDVTLPNLNTLYSAIQVLKVERIGHGIRVAESQELIDMVKEKNILLEVCPISNVLLKNAKSMDTHPIRQLYDAGVKVSVNSDDPGMFLTNINDDYEELYTHLNFTLEDFMKMNEWALEKSFMDSNIKDKIKNLYF

SEQ ID NO: 897 - protein, 363 aa, unidentified
  source 1..363: note = Plasmodium cynomolgi
MNILQEPIDFLKKDEIKNIDLSQMSKKERYKIWKRIPKCELHCHLDLCFSADFFLSCIRKYNLQPNLSDEEVLDYYLFAKGGKSLGEFVEKAIRVADIFHDYEVIEDLAKHAVFNKYKEGVVLMEFRYSPTFVAFKYKLDIELIHQAIVKGIKEVVELLDHKIHVALMCIGDTGHEAANIKASADFCLKHRADFVGFDHGGHEVDLKQYKEIFDYVRESGIPLSVHAGEDVTLPNLNTLYSAIQVLKVERIGHGIRVSESQELIDMVKEKNILLEVCPISNVLLKNAKSMDTHPIRQLYDAGVKVSVNSDDPGMFLTNINDDYEELYTHLNFTLEDFMKMNEWALEKSFMDSNIKDKVKNLYF

SEQ ID NO: 898 - protein, 363 aa, Homo sapiens
MAQTPAFDKPKVELHVHLDGSIKPETILYYGRRRGIALPANTAEGLLNVIGMDKPLTLPDFLAKFDYYMPAIAGCREAIKRIAYEFVEMKAKEGVVYVEVRYSPHLLANSKVEPIPWNQAEGDLTPDEVVALVGQGLQEGERDFGVKARSILCCMRHQPNWSPKVVELCKKYQQQTVVAIDLAGDETIPGSSLLPGHVQAYQEAVKSGIHRTVHAGEVGSAEVVKEAVDILKTERLGHGYHTLEDQALYNRLRQENMHFEICPWSSYLTGAWKPDTEHAVIRLKNDQANYSLNTDDPLIFKSTLDTDYQMTKRDMGFTEEEFKRLNINAAKSSFLPEDEKRELLDLLYKAYGMPPSASAGQNL

SEQ ID NO: 899 - protein, 352 aa, Mus musculus
MAQTPAFNKPKVELHVHLDGAIKPETILYFGKKRGIALPADTVEELRNIIGMDKPLSLPGFLAKFDYYMPVIAGCREAIKRIAYEFVEMKAKEGVVYVEVRYSPHLLANSKVDPMPWNQTEGDVTPDDVVDLVNQGLQEGEQAFGIKVRSILCCMRHQPSWSLEVLELCKKYNQKTVVAMDLAGDETIEGSSLFPGHVEAYEGAVKNGIHRTVHAGEVGSPEVVREAVDILKTERVGHGYHTIEDEALYNRLLKENMHFEVCPWSSYLTGAWDPKTTHAVVRFKNDKANYSLNTDDPLIFKSTLDTDYQMTKKDMGFTEEEFKRLNINAAKSSFLPEEEKKELLERLYREYQ

SEQ ID NO: 900 - protein, 363 aa, Bos taurus
MAQTPAFNKPKVELHVHLDGAIKPETILYYGRKRGIALPADTPEELQNIIGMDKPLSLPEFLAKFDYYMPAIAGCREAVKRIAYEFVEMKAKDGVVYVEVRYSPHLLANSKVEPIPWNQAEGDLTPDEVVSLVNQGLQEGERDFGVKVRSILCCMRHQPSWSSEVVELCKKYREQTVVAIDLAGDETIEGSSLFPGHVKAYAEAVKSGVHRTVHAGEVGSANVVKEAVDTLKTERLGHGYHTLEDATLYNRLRQENMHFEVCPWSSYLTGAWKPDTEHPVVRFKNDQVNYSLNTDDPLIFKSTLDTDYQMTKNEMGFTEEEFKRLNINAAKSSFLPEDEKKELLDLLYKAYGMPSPASAEQCL

SEQ ID NO: 901 - protein, 357 aa, Gallus gallus
MERGVRVFGEPKVELHIHLDGAIRPETILHFGKKRGVPLPGSTVDELMKHVSYQTPLSLKLFLEKFNHYMPAIAGDREAVRRIAYELVETKAKEGVVYVEVRYSPHLLANCRVEPIPWGQAEGDLTPEEVVNLVNQGLQDGERNFRIKARSILCCMRHMPSWSPEVVELCKKYQNNSVVAIDLAGDELLMASSDHKAAYEEAERCGIHRTVHAGEAGPATMIKEAVYLLKAERIGHGYHVLEDPELYRELLRTRMHFEVCPWSSYLTGACLPDFRKHPVVQFKKDQANYSINTDDPLIFNSNIDKDYGIVKEYMDFTEEDFKRVNINAAQSSFLPEKEKQELLNTLYEAYGMVPATS

SEQ ID NO: 902 - protein, 359 aa, Danio rerio
MNGKPAFDKPKVELHVHLDGAIRLKTVLDVAKRRGISLPVSMEEELKELCTVNEPATLTEFLGKFSHFMHVIAGDREAIKRIAYEFVETKAKEGVIYVEARYSPHFLANKGVEPLPWDQKPGDITPDDVVDLVNQGFKEGEQAFKTKARSILCCMRHMPNWSMEVVELCKKFHKDGVVAIDLAGDESMNCESYPGHKKAFEEAVRSNVHRTVHAGEVGPASVVREAVEVLKAERIGHGYHTLEDQNLYKQLLHQNMHFEMCPVSSRLTGACEPDFTKHPLITFKKDKANYSLNTDDPTIFNSTLNSDYEVVQKYMDFTEEEFKRLNINAAKSCFLPEKEKEKLLNQLYEAYGMRKSTSF

SEQ ID NO: 903 - protein, 358 aa, Xenopus laevis
MESKAFNKPKVELHVHLDGSIKPETIIHFAKKRQIKLPADTVEGLLEHVSYKEPLSLTEFLQKFNHYMPAIAGDREAIKRIAYEFVEMKAKEGVIYVEVRYSPHFLANSKVDPIPWGQKEGDITPDEVVDLVNQGLRKGEKTFNIKARSILCCMRHMPNWSSEVIELCKKYQNDTVVAIDLAGDESLNCESYPGHRKAYEEAVKCGIHRTVHAGEVGPPSVVKEAVEVLKAERIGHGYHTTEDPNLYKELLENNMHFEVCPWSSYLTSACHPDFTKHPATQFRKDKANFSLNTDDPLIFGSTLDVDYSIAVQHMGFTEDEFKRVNINAAKSSFLPDNEKKELLYKLYEAYGMILSTGL

SEQ ID NO: 904 - protein, 367 aa, unidentified
  source 1..367: note = Plasmodium falciparum
MNCKNMDTSYEIINYLTKDELDIDLSCMDKKERYKIWKRLPKCELHCHLDVCFSVDFFLNVIRKYNIQPNMSDEEIIDYYLFSKPGKSLDEFVEKALRLTDIYIDYTVVEDLAKHAVFNKYKEGVVLMEFRYSPSFMSFKHNLDKDLIHEAIVKGLNEAVALLEYKIQVGLLCTGDGGLSHERMKEAAEFCIKHKKDFVGYDHAGHEVDLKPFKDIFDNIREEGISLSVHAGEDVSIPNLNSLYTAINLLHVKRIGHGIRVSESQELIDLVKEKDILLEVCPISNVLLNNVKSMDTHPIRMLYDAGVKVSVNSDDPGMFLTNITDNYEELYTHLNFTLADFMKMNLWAVQKSFVDPDIKNKIISKYF

SEQ ID NO: 905 - protein, 337 aa, Drosophila melanogaster
MEQFLKGLPKVELHAHLNGSLGIKSLCDLGERLYGTSCKDFLKLCAHFSRFEKDMDACFEKFAFVHELTSTREGLRFATELAIRDFAEDNVQYVEMRTTPKANENYSRRDYLQIVIDAIKAASETYPEITVKLLPSINRAEPVDVAEETVSLAVELARAHPNLILGIDLSGNPGKGRFSDFAPILAQARDKGLKLAIHCAEIENPSEVKEMLHFGMSRCGHGTFLTPEDIGQLKQRNIAIECCLTSNVKSGTVPSLEEHHLKRIMEADAPKVICTDDSGVFDTTLTKEFLIAAETFGLTREQCIDLTLEAVHHSFASEQEQIQMADRVGNYADILVK

SEQ ID NO: 906 - protein, 358 aa, Xenopus tropicalis
MESKAFNKPKVELHVHLDGSIKPETIIHFAKKRQIKLPADTVEGLLEHVSYKEPLSLTEFLSKFNHYMPAIAGDREAIKRIAYEFVEMKAKEGVIYVEVRYSPHFLANSKVEPIPWGQKEGDITPDEVVDLVNQGLRKGEKAFNIKARSILCCMRHMPSWSTEVVELCKKYQNDTVVAIDLAGDESLNCESYPGHRKAYEEAVKCGIHRTVHAGEVGPSSVVKEAVEVLKAERIGHGYHTTEDPNLYKELLEKNMHFEVCPWSSYLTGACHPDFTKHPATQFRKDKANYSLNTDDPLIFGSTLDVDYSIAAKHMGFTEEEFKRVNINAAKSSFLPESEKKELLYKLYEAYGMILSTGL

SEQ ID NO: 907 - protein, 352 aa, Rattus norvegicus
MAQTPAFNKPKVELHVHLDGAIKPETILYYGKKRGIDLPADTVEGLRNIIGMDKPLSLPDFLAKFDYYMPAIAGCREAIKRIAYEFVEMKAKEGVVYVEVRYSPHLLANSKVDPIPWNQAEGDLTPDEVVDLVNQGLQEGEQAFGIKVRSILCCMRHQPSWSPEVLELCKKYHQKTVVAMDLAGDETIEGSSLFPGHVEAYEGAVKDGIHRTVHAGEVGSAEVVREAVDILKTERVGHGYHTIEDEALYNRLLKENMHFEVCPWSSYLTGAWNPKTTHAVVRFKDDQANYSLNSDDPLIFKSTVDTDYQMVKKDMGFTEEEFKRLNINAAKSSFLPEDEKKELLERLYKEYQ

SEQ ID NO: 908 - protein, 619 aa, Mus musculus
MATAGGSRRAPVPGPRLGLPLAAHLPASLGGEGAKDSVGGEKTSGNNDWFQSSRVPSFAQMLKKNLPVQPSAQTVTLPTGYSSESCSLSNMASKVTQVTGNFPEPLLSKGLSSISNPVLPPKKLPKEFIMKYKRGEINPVSALHQFAQMQRVQLDLKETVTTGNVMGPYFAFCAVVDGIQYKTGLGQNKKESRSNAAKLALDELLQLDEPEPRVLEPAGPPPIPAEPVVTPEAAYVSKVQYEGRQVQYAKISQLVKETFGQLISNHSQYLKCSSSLAAFIIERAGHHEVVAIGTGEYNYSQCIKPNGRVLHDTHAVVTARRSLLRYFYRQLLLFYSKNPAMMEKSIFCTEPASNLLTLKQNINLYLYMNQLPKGSAQIKSQLRLNPHSISAFEANEELSLHVAVEGKIYLTVYCSADGVNRVNSMSSSDKLTRWEVLGVQGALLSHFIQPVYISSILVGDGNCNDTRGLEIAINQRVDDALTSKLPMFYLVNRPHISLVPTAYPLQINLDHKSLSLNWAQGDNSLEIVDGLNGKITESSPFKSGLSMASRLCKAAMLSRFNLLAKEAKTDDLLEARTYHAAKCMSGPYQEAKALLKAYLQQHGYGSWIVKSPCIEQFSM

SEQ ID NO: 909 - protein, 619 aa, Rattus norvegicus
MATAGGSRRAPVPGPRLGLPLAAHLPASLGGEGAKDSLGGEKTSGNNDWFQSSRVPSFAQMLKKNLPVQSSAQTVTLPTGYSSESCSLSNMASKVTQVTGNFPEPLLSKGLSSISNPVLPPKKIPKEFIMKYRRGEINPVSALHQFAQMQRVQLDLKETVTTGNVMGPYFAFCAVVDGIQYKTGLGQNKKESRSNAAKLALDELLQLDEPEPRALEPAGPPPIPAEPIVTPEAAYISKVQYEGRQVQYAKISQLVKETFSQLISSHSQYLKCSSSLAAFIIERGGHHEVVAIGTGEYNYSQCIKPNGRVLHDTHAVVTARRSLLRYFYRQLLLFYSKNPAMMEKSIFCTEPASNLLTLKQNINLYLYMNQLPKGSAQIKSQLRLNPHSISAFEANEELSLHVAVEGKIYLTVYCSADGVNRVNSMSSSDKLTRWEVLGVQGALLSHFIQPVYISSILVGDGNCNDTRGLEIAINQRVDDALTSKLPMFYLVNRPHISLVPTAYPLQINLDHKSLSLNWAQGDNSLEIVDGLSGKITESSPFKSGLSMASRLCKAAMLSRFNLLAKEAKTDDLLEARTYHAAKCLSGPYQEAKALLKAYLQQHGYGSWIVKSPCIEQFSM

SEQ ID NO: 910 - protein, 586 aa, Xenopus laevis
MASNRNWSQHSSVPSFAQMLKKNLPDPGTSPAVNQTSTLSTCCLYNQPDCGTARVTRITGNFPEPFLSKMIVPPSLSSLTPRKVTKEFMVKYRRGDLNPISALYQFAQMQRMEIELKETVTTGNVFGAYFAFCAVVDGLEYKTGMGQNKKEAKANAAKLALDELLLHEDPALIDSENQSLNVIENPPPLPMNPRGTTETSTISRTRTDKRTFIHEKISSIIKETFTNLVSKYPEYENCGSSLAAFVIEKGGQHWEVVAIGTGEFNYGQSLQSDGRVLHDSHAMVVARRSLLRYFYRQLLLLYSGNNGMMDKSIFCTEPATNLLALKPNLNIFLYMNQLPKGAAQTNPQLCLSPHSLSAHEANDKLSLHVSVEGKNIPASYYSGEIVHNLYSMSSTDKLTRWEVLGVQGALLSIFIQPVYINSIIIGNAACSDTRGLEIAVKQRIDDALTSRLPMFYLVNRPYMSIVSSTHLTNNDTANKTLSLNWSQGDACVEVVDAAIGRTVEGSPFKSGSCLASRLCKAAMLCRFNLVVKESKRNAIPSGLSYHEAKRLAGPYQEAKCLLNSYFKQQGFGSWIAKPPIIGEFTM

SEQ ID NO: 911 - protein, 576 aa, Homo sapiens
MASNNHWFQSSQVPSFAQMLKKNLPVQPATKTITTPTGWSSESYGLSKMASKVTQVTGNFPEPLLSKNLSSISNPVLPPKKIPKEFIMKYKRGEINPVSALHQFAQMQRVQLDLKETVTTGNVMGPYFAFCAVVDGIQYKTGLGQNKKESRSNAAKLALDELLQLDEPEPRILETSGPPPFPAEPVVLSELAYVSKVHYEGRHIQYAKISQIVKERFNQLISNRSEYLKYSSSLAAFIIERAGQHEVVAIGTGEYNYSQDIKPDGRVLHDTHAVVTARRSLLRYFYRQLLLFYSKNPAMMEKSIFCTEPTSNLLTLKQNINICLYMNQLPKGSAQIKSQLRLNPHSISAFEANEELCLHVAVEGKIYLTVYCPKDGVNRISSMSSSDKLTRWEVLGVQGALLSHFIQPVYISSILIGDGNCSDTRGLEIAIKQRVDDALTSKLPMFYLVNRPHISLVPSAYPLQMNLEYKFLSLNWAQGDVSLEIVDGLSGKITESSPFKSGMSMASRLCKAAMLSRFNLLAKEAKKELLEAGTYHAAKCMSASYQEAKCKLKSYLQQHGYGSWIVKSPCIEQFNM

SEQ ID NO: 912 - protein, 351 aa, Bos taurus
MMEAEEQPWKTTFYSKLPKVELHAHLNGSISSNTIRKLIAKKPDLKIHDQMTMIDKGEKRTLEECLQMFQIIHLLTTTPEDVLMVTKDVIKEFADDGVKYLELRSTPRGEDATGMTKKTYVESILEGIKQSKEENVDIDVRYLISIDRRGGSSAAKEAVKLAEEFFLSAEDTVLGLDLSGDPSAGQAKDFLEPLLEAKKSGLKLALHLSEIPNQKTETQVLLNLFPDRIGHGTFLSSSEEGSPDLVDFVRQHQIPLELCLTSNVKSQTVPAYDQHHFGFWYSVAHPAVICTDDKGVFATRLSQEYQLVAETFHLTQSQVWDLSYESISYIFASDSTKADLRKKWSHLKPHF

SEQ ID NO: 913 - protein, 348 aa, Danio rerio
MDTEADLFYRQLPKVELHAHLNGSVSFETMEKLIKRKPHLNIEHSMTAIRRGQRRTLDECFQVFKVIHQLVDSEEDILMVAKSVIQEFAADGVKYLELRSTPREVTETGLSKQRYIETVLEAIRQCKQEGVDIDVRFLVAVDRRHGPEVAMQTVKLAEDFLLSSDGTVVGLDLSGDPTVGHGKDLLAALQKAKNCGLKLALHLSEVPSQIDETELLLNLPPDRIGHGTFLHPDVGGSDSLVDKVCKQNIPIEICLTSNVKGQTVPSYDKHHFKYWYNRGHPCVLCTDDKGVFCTDLSQEYQLAASTFGLTKEAVWILSQQAIGYTFAPEPIKQRLEKTWAELKQQILQ

SEQ ID NO: 914 - protein, 347 aa, Xenopus laevis
MAGEGALQFYRDLPKVELHAHLNGSISTATMKKLMARKPHLDIQHGMTMIDKGQKRTLEECFQMFKIIHQITDTAEDILLVTKDVIKEFAADGVKYLELRSTPRDTPAGLTKQAYVETVLEGIKQCKEEGVDIDVRFLLAIDRRGGPTAAKETVKLAEDFFCSSNELVLGLDLSGDPTVGHGRDFMEPLNKARQSGLKLALHLSEIPSQTEETELLLGLPPDRIGHGTFLTTSAHIVEIVKKQHIPLELCITSNIKGQTVSSYNEHHFGFWYNLHHPFVLCTDDKGVFATDLSVEYEIAAKTFNLTPHHVWDLSYQAIDYTFASADVKANLKEKWLLLKPDVFRHAL

SEQ ID NO: 915 - protein, 355 aa, Homo sapiens
MIEAEEQQPCKTDFYSELPKVELHAHLNGSISSHTMKKLIAQKPDLKIHDQMTVIDKGKKRTLEECFQMFQTIHQLTSSPEDILMVTKDVIKEFADDGVKYLELRSTPRRENATGMTKKTYVESILEGIKQSKQENLDIDVRYLIAVDRRGGPLVAKETVKLAEEFFLSTEGTVLGLDLSGDPTVGQAKDFLEPLLEAKKAGLKLALHLSEIPNQKKETQILLDLLPDRIGHGTFLNSGEGGSLDLVDFVRQHRIPLELCLTSNVKSQTVPSYDQHHFGFWYSIAHPSVICTDDKGVFATHLSQEYQLAAETFNLTQSQVWDLSYESINYIFASDSTRSELRKKWNHLKPRVLHI

SEQ ID NO: 916 - protein, 388 aa, Caenorhabditis elegans
MPNNSKHKKKQQRRQQEAQKKSRAKQIETDKKNDEFLDTELDEVSPLVIDDDMTEFKNMPKVELHAHLSGSLSPETIKLIMESDETRAEEIMKKYKLEKPENMTGVFDCFPVIHAILRKPEAIRIAIRQTIKEFEEDNCVYLELRTSPKETDFMTYEDYLQVCIESFEAAKHEFPRIKTFLIVSLDRRMPFETAAHILGLIGEAQQRTNVIVGVELSGDPHLDGRRLLKLFVAARRFHGLGITIHLAEVLQNMADVEDYLNLRPDRIGHGTFLHTDPYTEYLTNKYKIPLEICLSSNVYSKTTTNYRNSHFNYWRKRGVPVFICTDDKGVIPGATLTEEYYKAAITFDLSTEELIGINQDALLNSFAYKYNVTDLTETFRKINNNVLD

SEQ ID NO: 917 - protein, 360 aa, Mus musculus
MEAGQQWPGKTDFYLQLPKVELHAHLNGSISSSTMKKLIAKKPHLNVHGHMTMIDKGKKRTLQECFQMFQVIHQLTTSAEDILMVTKDVIKEFADDGVKYLELRSTPREENATGMTRKTYVESVLEGIKQCKQENLDIDVRYLMAIDRRGGPTIARETVELAKEFFLSTENTVLGLDLSGDPTIGQANDFLEPLLEAKKAGLKLALHLAEIPNREKENQMLLSLLPDRIGHGTFLSASEAGALDQVDFVRQHQIPLELCLTSNIKSQTVPSYDQHHFGFWYSIAHPSVICTDDKGVFATYLSQEYQLAAETFNLTPFQVWDLSYESINYIFACDNTRSELRKRWTHLKQKVLNCNEVNYF

SEQ ID NO: 918 - protein, 583 aa, Homo sapiens
MASASQGADDDGSRRKPRLAASLQISPQPRPWRPLPAQAQSAWGPAPAPATYRAEGGWPQVSVLRDSGPGAGAGVGELGAARAWENLGEQMGKAPRVPVPPAGLSLPLKDPPASQAVSLLTEYAASLGIFLLFREDQPPGPCFPFSVSAELDGVVCPAGTANSKTEAKQQAALSALCYIRSQLENPESPQTSSRPPLAPLSVENILTHEQRCAALVSAGFDLLLDERSPYWACKGTVAGVILEREIPRARGHVKEIYKLVALGTGSSCCAGWLEFSGQQLHDCHGLVIARRALLRFLFRQLLLATQGGPKGKEQSVLAPQPGPGPPFTLKPRVFLHLYISNTPKGAARDIYLPPTSEGGLPHSPPMRLQAHVLGQLKPVCYVAPSLCDTHVGCLSASDKLARWAVLGLGGALLAHLVSPLYSTSLILADSCHDPPTLSRAIHTRPCLDSVLGPCLPPPYVRTALHLFAGPPVAPSEPTPDTCRGLSLNWSLGDPGIEVVDVATGRVKANAALGPPSRLCKASFLRAFHQAARAVGKPYLLALKTYEAAKAGPYQEARRQLSLLLDQQGLGAWPSKPLVGKFRN

SEQ ID NO: 919 - protein, 561 aa, Mus musculus
MASVDEGGRRRPRLAASLQISPGPWKPSGGQEPTEAGDAAPRTAEHGVAGAQEAHREACKALGGSVLSPGPAGDFPGALHGLSMLPKDPPPAQAVALLTQCMANLGVSLTFLEDQTAGPGSSFSVCADLDGLVCPAGTGSSKLEAKQQAALSALQYIQKQLERPEPLVTPRQPLLTSLSIETILTHEQRCAAVVSAGLDRLLSESSPYQACKGTVAAVILEREVQGSIGHSKETYELVALGTGSSSCAGWLEFSGRRLHDCHGLVIARRALLRFFFRQLLLVTQGGPKGQERSVLTPQPGPGPPFALKPGVFLHLYVSNTPKGAAHDIYLPLASEDSVLHSPAFRLQAHVCGQLKPVSYVAPALRDTHVGCLSASDKLARWAILGLGGGLLAHFLPPLYATSLVLADPCHDPSTLNRVIHSRPRLDSVLGSCLPCPYVRTTLHLFAGPLVAPSDPGPSTCHSLSLNWSLGDPDIEVVDVATGRVKTDSSVGPPSRLCKAAFLSAFRQVARALEKPQLLSLQTYEAAKAVPYREARQQLSLLLDQQGLGAWPSKPLVGKFRH

SEQ ID NO: 920 - protein, 580 aa, Macaca fascicularis
MASASQGADDGSRRKPRLAASLQISPEPRPWRPLPPQAQGAWEPAPAMDHAEGGQPQVSVLRDSGPGAGAGVGELGAAQAWENLGEQMGRTPRVPVPPAGLSLPLKDPPASQAVSLLTEYAASLGIILLFREDQQPGPCFPFSVSAELDGVVCPAGTANSKTEAKQQAALSALCYIRSQLESPESPQTSSRPPPPPLSVDSILTHGQRCAALVSAGFDLLLDERSPYWACKGTVAGVILEREIPGARGHVKEIYKLVALGTGSSCCAGWLEFSGQQLHDCHGLVIARRALLRFLFRQLLLATQGGAKGKEQSVLAPQPGPGPPFTLKPRVFLHLYISNTPKGAARDIYLPPTSEGGLPHSPPMRLQAHVLGQLKPVCYVAPSLCDTHVGCLSASDKLARWAVLGLGGALLAHLVSPLYSTSLILADSCHDPPTLSRAIHTRPCLDSVLGPCLPPPYVRTALHLFSGPPVAPSEPTPDTCHGLSLNWSLGDPGIEVVDVATGRVKANAALGPPSRLCKASFLRAFHQVARAVGKPYLLALKTYEAAKAGPYQEARRQLSLLLDQQGLGAWPSKPLVGKFRN

(19) United States
(12) Patent Application Publication
(10) Pub. No.: US 2026/0152740 A1
(43) Pub. Date: Jun. 4, 2026

(54) GENE EDITING SYSTEMS AND METHODS FOR REDUCING IMMUNOGENICITY AND GRAFT VERSUS HOST RESPONSE

(71) Applicant: CORRECTSEQUENCE THERAPEUTICS (SHANGHAI) CO., LTD, Shanghai (CN)

(72) Inventors: Peixue LI, Shanghai (CN); Qing SU, Shanghai (CN); Lijie WANG, Shanghai (CN); Yichuan WANG, Shanghai (CN); Ya XU, Shanghai (CN); Jing HE, Shanghai (CN); Huanyu LI, Shanghai (CN); Xiaodun MOU, Shanghai (CN)

(73) Assignee: CORRECTSEQUENCE THERAPEUTICS (SHANGHAI) CO., LTD, Shanghai (CN)

(21) Appl. No.: 19/124,014

(22) PCT Filed: Oct. 27, 2023
(86) PCT No.: PCT/CN2023/127220

§ 371 Date: Apr. 24, 2025

(30) Foreign Application Priority Data
Oct. 28, 2022 (WO) PCT/CN2022/128215
Mar. 10, 2023 (WO) PCT/CN2023/080709

(51) Int. Cl. C12N 15/11; C12N 9/22; C12N 9/50

(52) U.S. Cl. CPC C12N 15/11; C12N 9/226; C12N 9/506; C12Y 304/22044; C07K 2319/50; C07K 2319/85; C12N 2310/20

(57) ABSTRACT

Provided are gene editing systems and methods for reducing cell immunogenicity and reducing graft versus host (GvH) response, or to promote the persistence of therapeutic cells. Also provided are polynucleotides, vectors, cells, kits and compositions comprising components of the gene editing systems, and methods related to treatment for reducing cell immunogenicity and reducing GvH response.

### FIELD OF DISCLOSURE

The present disclosure generally relates to gene editing systems and methods for reducing immunogenicity and/or reducing graft versus host (GvH) responses, and/or promoting persistence of therapeutic cells. Also disclosed are polynucleotides, vectors, cells, kits, and compositions comprising components of the gene editing systems, and methods related to treatment for reducing immunogenicity and/or reducing GvH responses and/or promoting persistence of therapeutic cells.

### CROSS REFERENCE TO RELATED APPLICATION

This application claims the priority to and benefits of International Application No. PCT/CN2022/128215, filed Oct. 28, 2022, and International Application No. PCT/CN2023/080709, filed Mar. 10, 2023, which are incorporated herein by reference in its entirety.

### SEQUENCE LISTING

This application contains a Sequence Listing electronically submitted as an XML file entitled “sequence listing.xml” having a size of 1,053,655 bytes and created on Oct. 27, 2023. The information contained in the Sequence Listing is incorporated by reference herein.

### BACKGROUND

CAR-T cell therapy, which emerged as a promising immunotherapy for tumors, has revolutionized antitumor treatments, especially for hematological malignancies, where it leads to remarkable, long-term antineoplastic effects with higher target specificity. Several autologous CAR-T cell therapy products have been approved by the U.S. Food and Drug Administration (FDA), the European Medicines Agency (EMA), or China's National Medical Products Administration (NMPA), mainly to treat refractory or relapsed lymphoma or myeloma. (Ghaffari, Sasan, Nastaran Khalili, and Nima Rezaei. “CRISPR/Cas9 revitalizes adoptive T-cell therapy for cancer immunotherapy.” Journal of Experimental & Clinical Cancer Research 40.1 (2021): 269.)

However, there are some limitations of the regular autologous CAR-T cell therapy, such as long manufacturing periods, manufacturing failures in some patients, and high cost, which cast a shadow over the development of autologous CAR-T cell therapy. Thus, the development of a universal/allogenic CAR-T (UCAR-T) or enhanced autologous CAR-T cell therapy is an attractive breakthrough point that may overcome some of these drawbacks.

Despite the advantages over autologous CAR-T therapy, there are two major challenges for allogenic CAR-T cell therapy. First, the administered allogeneic T cells may cause life-threatening graft-versus-host disease (GvHD). Second, allogeneic T cells may be rapidly eliminated by the host immune system, limiting their persistence of antitumour activity.

GvHD is one of the main causes of death after allogeneic hematopoietic stem cell transplantation, so it must be prevented. Because T-cell alloreactivity is dependent on the interaction of T-cell receptors (TCRs) with alloantigens presented by human leukocyte antigens (HLAs), TCR-depleted T cells do not cause GvH responses when infused into HLA-unmatched patients. Some studies tried to reduce the risk of GvHD by genetic ablation of the TCR locus, mainly the TCRα constant region (TRAC), using small interfering RNA, ZFN, TALEN, megaTAL nucleases, engineered homing endonucleases, or CRISPR/Cas9. On the other hand, to prevent or reduce host immune rejection, one way is to reduce the immunogenicity of the allogenic T cells, such as by genetic abrogation of the b2-microglobulin (B2M) gene to disrupt the MHC class I molecules. Another way is to delete the CD52 of the donor T cells and use anti-CD52 monoclonal antibody to eliminate host T cells (which express CD52) to avoid allorejection. In addition, inhibitory checkpoints (e.g., PD-1, coded by the PDCD1 gene, CTLA4 gene, TIGIT gene, LAG3 gene, and TIM-3 gene) can be knocked out separately or simultaneously with each other, and/or with TRAC, CD52, B2M, PDCD1, CTLA4, TIGIT, TIM3, LAG3, CISH, TGFBR2, FAS, CD7, CBLB, KLRC1, and/or CD38 to enhance the efficacy and persistence of the autologous or allogenic CAR-T cells or NK cells. CD7 or CD38 could be knocked out for anti-CD7 or anti-CD38 CAR-T cells to prevent fratricide. FAS/FASL signal induces apoptosis of cytotoxic T cells, which dampens the anti-tumor efficacy of CAR-T therapy. TGF-β secreted in the tumor microenvironment (TME) suppresses T cell function by binding to TGFBR2. Cytokine-induced SH2 (CISH) protein is induced in CD8+ T-cells upon TCR stimulation and inhibits T-cell anti-tumor function. CISH is also a key negative regulator of IL-15 signaling in NK cells. CBLB has been characterized as an intracellular checkpoint in T cells and also in NK cells and deletion of CBLB enhances the function of T and NK cells. KLRC1 gene encodes the NK cell inhibitory receptor NKG2A, which is a potent NK cell immune checkpoint.

While most groups are using nuclease (ZNF, TALEN, or CRISPR/Cas9) for knockout of these targets, there are reports concerning the risk of these modifications as they all cause double strand breaks (DSB) and will activate the p53 pathway, affect cell growth, and induce chromosome translocation. The risks will further increase with duplex or multiplex editing. Thus, there is an unmet need for the development of safer methods for modifying these targets.

The combination of CRISPR-Cas9 and cytidine deaminases (APOBEC/AID) leads to as cytosine base editors (CBEs) for programmable cytosine to thymine (C-to-T) substitutions. Such CBEs have been applied to achieve efficient editing in various species successfully. Because such a base editing process does not depend on the generation of DNA double strand break (DSB), unwanted nucleotide insertions/deletions (indels) or DNA damage responses (DDRs) can be largely avoided.

The safety and efficiency of gene editing tools are of great importance in clinical applications. Although the CBEs do not cause DSB or activate a p53-mediated DDR pathway as Cas9 nuclease, the APOBEC/AID family members can trigger C-to-T base substitutions in single-stranded DNA (ssDNA) regions, which are formed randomly during various cellular processes, including DNA replication, repair, and transcription. Thus, the specificity of previous base editing systems is compromised, which may limit the applications of CBEs for therapeutic purposes.

### SUMMARY

The present disclosure provides gene editing systems, polynucleotides, vectors, cells, compositions, kits, and methods to reduce immunogenicity and graft versus host response. In some embodiments, the present disclosure provides gene editing systems targeting a gene selected from TRAC, CD52, B2M, PDCD1, CTLA4, TIGIT, TIM3, LAG3, CISH, TGFBR2, FAS, CD7, CBLB, KLRC1 and CD38.

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a T-cell receptor a constant (TRAC) gene and the hgRNA comprises a hgRNA spacer, wherein the nucleic acid sequence of the mgRNA spacer comprises a sequence selected from SEQ ID NOs: 1-5.

In some embodiments, the nucleic acid sequences of the mgRNA spacer and the hgRNA spacer comprise the sequences as set forth in Table 2. In some embodiments, the nucleic acid sequences of the mgRNA and the hgRNA comprise the sequences as set forth in Table 26.

In another aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a CD52 gene and the hgRNA comprises a hgRNA spacer, wherein the nucleic acid sequence of the mgRNA spacer comprises a sequence selected from SEQ ID NOs: 6-8.

In some embodiments, the nucleic acid sequences of the mgRNA spacer and the hgRNA spacer comprise the sequences as set forth in Table 3. In some embodiments, the nucleic acid sequences of the mgRNA and the hgRNA comprise the sequences as set forth in Table 27.

In another aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a b2-microglobulin (B2M) gene and the hgRNA comprises a hgRNA spacer, wherein the nucleic acid sequence of the mgRNA spacer comprises a sequence selected from SEQ ID NOs: 9-19.

In some embodiments, the nucleic acid sequences of the mgRNA spacer and the hgRNA spacer comprise the sequences as set forth in Table 4. In some embodiments, the nucleic acid sequences of the mgRNA and the hgRNA comprise the sequences as set forth in Table 28.

In another aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a programmed cell death protein 1 (PDCD1) gene and the hgRNA comprises a hgRNA spacer, wherein the nucleic acid sequence of the mgRNA spacer comprises a sequence selected from SEQ ID NOs: 20-38.

In some embodiments, the nucleic acid sequences of the mgRNA spacer and the hgRNA spacer comprise the sequences as set forth in Table 5. In some embodiments, the nucleic acid sequences of the mgRNA and the hgRNA comprise the sequences as set forth in Table 29.

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a cytotoxic T-lymphocyte associated protein 4 (CTLA4) gene and the hgRNA comprises a hgRNA spacer, wherein the nucleic acid sequence of the mgRNA spacer comprises a sequence selected from SEQ ID NOs: 247-256.

In some embodiments, the nucleic acid sequences of the mgRNA spacer and the hgRNA spacer comprise the sequences as set forth in Table 13.

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a T cell immunoreceptor with Ig and ITIM domains (TIGIT) gene and the hgRNA comprises a hgRNA spacer, wherein the nucleic acid sequence of the mgRNA spacer comprises a sequence selected from SEQ ID NOs: 278-294.

In some embodiments, the nucleic acid sequences of the mgRNA spacer and the hgRNA spacer comprise the sequences as set forth in Table 14.

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a hepatitis A virus cellular receptor 2 (HAVCR2/TIM3) gene and the hgRNA comprises a hgRNA spacer, wherein the nucleic acid sequence of the mgRNA spacer comprises a sequence selected from SEQ ID NOs: 323-337.

In some embodiments, the nucleic acid sequences of the mgRNA spacer and the hgRNA spacer comprise the sequences as set forth in Table 15.

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a as lymphocyte activating 3 (LAG3) gene and the hgRNA comprises a hgRNA spacer, wherein the nucleic acid sequence of the mgRNA spacer comprises a sequence selected from SEQ ID NOs: 364-396.

In some embodiments, the nucleic acid sequences of the mgRNA spacer and the hgRNA spacer comprise the sequences as set forth in Table 16.

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a cytokine inducible SH2 containing protein (CISH) gene and the hgRNA comprises a hgRNA spacer, wherein the nucleic acid sequence of the mgRNA spacer comprises a sequence selected from SEQ ID NOs: 472-482.

In some embodiments, the nucleic acid sequences of the mgRNA spacer and the hgRNA spacer comprise the sequences as set forth in Table 17. In some embodiments, the nucleic acid sequences of the mgRNA and the hgRNA comprise the sequences as set forth in Table 30.

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a transforming growth factor beta receptor 2 (TGFBR2) gene and the hgRNA comprises a hgRNA spacer, wherein the nucleic acid sequence of the mgRNA spacer comprises a sequence selected from SEQ ID NOs: 504-510.

In some embodiments, the nucleic acid sequences of the mgRNA spacer and the hgRNA spacer comprise the sequences as set forth in Table 18. In some embodiments, the nucleic acid sequences of the mgRNA and the hgRNA comprise the sequences as set forth in Table 31.

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a Fas cell surface death receptor (FAS) gene and the hgRNA comprises a hgRNA spacer, wherein the nucleic acid sequence of the mgRNA spacer comprises a sequence selected from SEQ ID NOs: 530-541.

In some embodiments, the nucleic acid sequences of the mgRNA spacer and the hgRNA spacer comprise the sequences as set forth in Table 19. In some embodiments, the nucleic acid sequences of the mgRNA and the hgRNA comprise the sequences as set forth in Table 32.

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a CD7 gene and the hgRNA comprises a hgRNA spacer, wherein the nucleic acid sequence of the mgRNA spacer comprises a sequence selected from SEQ ID NOs: 565-575.

In some embodiments, the nucleic acid sequences of the mgRNA spacer and the hgRNA spacer comprise the sequences as set forth in Table 20.

In an aspect, the present disclosure provides a gene editing system comprising a main guide RNA (mgRNA) and a helper guide RNA (hgRNA), or at least one DNA polynucleotide encoding the mgRNA and/or the hgRNA, wherein the mgRNA comprises a mgRNA spacer targeting a Cb1 proto-oncogene B (CBLB) gene and the hgRNA comprises a hgRNA spacer, wherein the nucleic acid sequence of the mgRNA spacer comprises a sequence selected from SEQ TD NOs: 609-618.

In some embodiments, the nucleic acid sequences of the mgRNA spacer and the hgRNA spacer comprise the sequences as set forth in Table 21. In some embodiments, the nucleic acid sequences of the mgRNA and the hgRNA comprise the sequences as set forth in Table 33.

In an aspect, the present disclosure provides a gene editing system comprising a main guide RNA (mgRNA) and a helper guide RNA (hgRNA), or at least one DNA polynucleotide encoding the mgRNA and/or the hgRNA, wherein the mgRNA comprises a mgRNA spacer targeting a killer cell lectin like receptor C1 (KLRC1) gene and the hgRNA comprises a hgRNA spacer, wherein the nucleic acid sequence of the mgRNA spacer comprises a sequence selected from SEQ ID NOs: 637-641.

In some embodiments, the nucleic acid sequences of the mgRNA spacer and the hgRNA spacer comprise the sequences as set forth in Table 22. In some embodiments, the nucleic acid sequences of the mgRNA and the hgRNA comprise the sequences as set forth in Table 34.

In an aspect, the present disclosure provides a gene editing system comprising a main guide RNA (mgRNA) and a helper guide RNA (hgRNA), or at least one DNA polynucleotide encoding the mgRNA and/or the hgRNA, wherein the mgRNA comprises a mgRNA spacer targeting a CD38 gene and the hgRNA comprises a hgRNA spacer, wherein the nucleic acid sequence of the mgRNA spacer comprises a sequence selected from SEQ ID NOs: 651-659.

In some embodiments, the nucleic acid sequences of the mgRNA spacer and the hgRNA spacer comprise the sequences as set forth in Table 23. In some embodiments, the nucleic acid sequences of the mgRNA and the hgRNA comprise the sequences as set forth in Table 35.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, and (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, and wherein the first Cas protein and second Cas protein are the as same or different.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, and (6) a protease, or a polynucleotide encoding the protease, and (7) a nucleobase deaminase inhibitor domain, wherein the first Cas protein and second Cas protein are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, (6) a protease, or a polynucleotide encoding the protease, (7) a nucleobase deaminase inhibitor domain, and (8) a second fusion protein comprising the protease and a second RNA binding domain, or a polynucleotide encoding the second fusion protein, wherein the first Cas protein and second Cas protein are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof, wherein the protease and the second RNA binding domain are optionally connected by a linker, wherein the mgRNA further comprises a second protein-binding motif, and wherein the second RNA binding domain binds to the second protein-binding motif.

In some embodiments, the protease is split into a first protease fragment and a second as protease fragment, wherein the first or second protease fragment alone is not able to cleave the cleavage site.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, (6) a protease, or a polynucleotide encoding the protease, (7) a nucleobase deaminase inhibitor domain, (8) a second fusion protein comprising the first protease fragment and a second RNA binding domain, or a polynucleotide encoding the second fusion protein, wherein the first protease fragment and the second RNA binding domain are optionally connected by a linker, and (9) a third fusion protein comprising the second protease fragment and a third RNA binding domain, or a polynucleotide encoding the third fusion protein, wherein the second protease fragment and the third RNA binding domain are optionally connected by a linker, wherein the first Cas protein and second Cas protein are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof, wherein the mgRNA further comprises a second protein-binding motif and a third protein-binding motif, wherein the second RNA binding domain binds to the second protein-binding motif, and wherein the third RNA binding domain binds to the third protein-binding motif.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, (6) a protease, or a polynucleotide encoding the protease, (7) a nucleobase deaminase inhibitor domain, (8) a second fusion protein comprising the first protease fragment as and a second RNA binding domain, or a polynucleotide encoding the second fusion protein, wherein the first protease fragment and the second RNA binding domain are optionally connected by a linker, and (9) a third fusion protein comprising the second protease fragment and a third RNA binding domain, or a polynucleotide encoding the third fusion protein, wherein the second protease fragment and the third RNA binding domain are optionally connected by a linker, wherein the first Cas protein and second Cas protein are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof, wherein the mgRNA further comprises a second protein-binding motif and a third protein-binding motif, wherein the second RNA binding domain binds to the second protein-binding motif, wherein the third RNA binding domain binds to the third protein-binding motif, and wherein the second and third RNA binding domains are the same or different, and the second and third protein-binding motifs are the same or different.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, (6) a protease, or a polynucleotide encoding the protease, (7) a nucleobase deaminase inhibitor domain, and (8) a second fusion protein comprising the first protease fragment and a second RNA binding domain, or a polynucleotide encoding the second fusion protein, wherein the first Cas protein and second Cas protein are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof, wherein the first protease fragment and the second RNA binding domain are optionally connected by a linker, wherein the mgRNA further comprises a second protein-binding motif, and wherein the second RNA binding domain binds to the second protein-binding motif.

In some embodiments, the protease is a TEV protease, a TuMV protease, a PPV protease, a PVY protease, a ZIKV protease, or a WNV protease.

In some embodiments, the protease is a TEV protease. In some embodiments, the TEV protease comprises a sequence as set forth in SEQ ID NO: 124.

In some embodiments, the first TEV protease fragment comprises a sequence of SEQ ID NO: 125.

In some embodiments, the nucleobase deaminase inhibitor is an inhibitory domain of a nucleobase deaminase.

In some embodiments, the nucleobase deaminase inhibitor is an inhibitory domain of a cytidine deaminase.

In some embodiments, the inhibitory domain of a cytidine deaminase comprises an amino acid sequence as set forth in SEQ ID NO: 141 or SEQ ID NO: 142.

In some embodiments, the nucleotide deaminase is a cytidine deaminase.

In some embodiments, the cytidine deaminase is selected from the group consisting of APOBEC3B (A3B), APOBEC3C (A3C), APOBEC3D (A3D), APOBEC3F (A3F), APOBEC3G (A3G), APOBEC3H (A3H), APOBECI (A1), APOBEC3 (A3), APOBEC2 (A2), APOBEC4 (A4), and AICDA (AID).

In some embodiments, the cytidine deaminase is a human or mouse cytidine deaminase.

In some embodiments, the catalytic domain of the cytidine deaminase is a mouse A3 cytidine deaminase domain 1 (mA3-CDA1) or human A3B cytidine deaminase domain 2 (hA3B-CDA2).

In some embodiments, the first fusion protein further comprises an uracil glycosylase inhibitor (UGI).

In some embodiments, the first fusion protein further comprises a nuclear localization sequence (NLS).

In some embodiments, the Cas protein is a Cas9, a dead Cas9 (dCas9), or a Cas9 nickase (nCas9) selected from the group consisting of SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, and RanCas13b.

In some embodiments, the Cas protein is a nCas9. In some embodiments, the nCas9 protein is a nCas9-D10A protein. In some embodiments, the nCas9-D10A protein has an amino acid sequence of SEQ ID NO: 146.

In some embodiments, the first protein-binding RNA motif and the first RNA binding domain, the second protein-binding RNA motif and the second RNA binding domain, and the third protein-binding RNA motif and the third RNA binding domain, are each independently selected from the group consisting of a MS2 phage operator stem-loop and MS2 coat protein (MCP) or an RNA-binding section thereof; a BoxB and N22P or an RNA-binding section thereof; a telomerase Ku binding motif and Ku protein or an RNA-binding section thereof; a telomerase Sm7 binding motif and Sm7 protein or an RNA-binding section thereof; a PP7 phage operator stem-loop and PP7 coat protein (PCP) or an RNA-binding section thereof; a SfMu phage Coin stem-loop and Com RNA binding protein or an RNA-binding section thereof; and a non-natural RNA aptamer and corresponding aptamer ligand or an RNA-binding section thereof.

In another aspect, the present disclosure provides a polynucleotide encoding the hgRNA and/or the mgRNA disclosed herein.

In another aspect, the present disclosure provides a polynucleotide encoding all components except the first and the second Cas protein in the gene editing system disclosed herein.

In another aspect, the present disclosure provides a kit comprising a polynucleotide encoding all components except the first and the second Cas protein in the gene editing system disclosed herein, and a polynucleotide encoding the first and/or second Cas protein in the gene editing system disclosed herein. In some embodiments, the first and the second Cas proteins are the same Cas protein.

In another aspect, the present disclosure provides a vector comprising the polynucleotide encoding the hgRNA and/or the mgRNA disclosed herein.

In another aspect, the present disclosure provides a vector comprising the polynucleotide encoding all components except the first and the second Cas protein in the gene editing system disclosed herein.

In some embodiments, the vector is a plasmid or a viral vector.

In some embodiments, the vector is a polycistronic vector.

In another aspect, the present disclosure provides a kit comprising the vector disclosed above, and a vector comprising the polynucleotide encoding the first and/or second Cas protein in the gene editing system disclosed herein.

In another aspect, the present disclosure provides a cell comprising any one or more of the gene editing systems disclosed herein.

In another aspect, the present disclosure provides a cell comprising the polynucleotide disclosed herein. In some embodiments, the cell further comprises a polynucleotide encoding the first and/or second Cas protein in the gene editing system disclosed herein.

In another aspect, the present disclosure provides a cell comprising the vector disclosed herein. In some embodiments, the cell further comprises a vector comprising a polynucleotide encoding the first and/or second Cas protein in the gene editing system disclosed herein.

In another aspect, the present disclosure provides a cell comprising the components of the kit disclosed herein.

In some embodiments, the cell is a stem cell.

In some embodiments, the cell is a pluripotent stem cell, or a hematopoietic stem cell.

In some embodiments, the pluripotent stem cell is an induced pluripotent stem cell (iPSC) or an embryonic stem cell.

In some embodiments, the cell is an immune cell.

In some embodiments, the cell is selected from the group consisting of T cell, B cell, natural killer cell (NK cell), macrophage, dendritic cell, monocyte, granulocyte, and mast cell.

In some embodiments, the cell is a T cell.

In some embodiments, the T cell comprises a chimeric antigen receptor (CAR).

In some embodiments, the T cell is a CAR-T cell.

In some embodiments, the cell is a natural killer cell (NK cell).

In some embodiments, the NK cell is a CAR-NK cell.

In some embodiments, the cell is a primary cell.

In some embodiments, the cell is a differentiated cell.

In some embodiments, the cell is differentiated from a pluripotent stem cell. In some embodiments, the cell is differentiated from an iPSC or an ESC.

In another aspect, the present disclosure provides a composition comprising any one or more of the gene editing systems disclosed herein.

In another aspect, the present disclosure provides a composition comprising the cell disclosed herein.

In another aspect, the present disclosure provides a kit comprising one or more of the gene editing systems disclosed herein. For example, the present disclosure provides a kit comprising a first gene editing system targeting the PDCD1 gene, and a second gene editing system targeting the TRAC gene, the B2M gene, and/or the CD52 gene. For example, in some embodiments, the present disclosure provides a kit comprising a first gene editing system and a second gene editing system, wherein the first gene editing system and the second gene editing system each targets a gene selected from TRAC, CD52, B2M, PDCD1, CTLA4, TIGIT, TIM3, LAG3, CISH, TGFBR2, FAS, CD7, CBLB, KLRC1, and CD38.

In some embodiments, the present disclosure provides a kit comprising a first gene editing system targeting the TRAC gene, and a second gene editing system targeting a gene selected from CD52, B2M, PDCD1, CTLA4, TIGIT, TIM3, LAG3, CISH, TGFBR2, FAS, CD7, CBLB, and CD38.

In some embodiments, the present disclosure provides a kit comprising a first gene editing system targeting the CD52 gene, and a second gene editing system targeting a gene selected from PDCD1, CTLA4, TIGIT, TIM3, LAG3, CISH, TGFBR2, FAS, CD7, CBLB, KLRC1, and CD38.

In some embodiments, the present disclosure provides a kit comprising a first gene editing system targeting the B2M gene, and a second gene editing system targeting a gene selected from PDCD1, CTLA4, TTGIT, TIM3, LAG3, CISH, TGFBR2, FAS, CD7, CBLB, KLRC1, and CD38.

In some embodiments, the present disclosure provides a kit comprising a first gene editing system targeting the PDCD1 gene, and a second gene editing system targeting a gene selected from CTLA4, TIGIT, TIM3, LAG3, CISH, TGFBR2, FAS, CD7, CBLB, KLRC1, and CD38.

In some embodiments, the present disclosure provides a kit comprising a first gene editing system targeting the CTLA4 gene, and a second gene editing system targeting a gene as selected from TIGIT, TIM3, LAG3, CISH, TGFBR2, FAS, CD7, CBLB, KLRC1, and CD38.

In some embodiments, the present disclosure provides a kit comprising a first gene editing system targeting the TIGIT gene, and a second gene editing system targeting a gene selected from TIM3, LAG3, CISH, TGFBR2, FAS, CD7, CBLB, KLRC1, and CD38.

In some embodiments, the present disclosure provides a kit comprising a first gene editing system targeting the TIM3 gene, and a second gene editing system targeting a gene selected from LAG3, CISH, TGFBR2, FAS, CD7, CBLB, KLRC1, and CD38.

In some embodiments, the present disclosure provides a kit comprising a first gene editing system targeting the LAG3 gene, and a second gene editing system targeting a gene selected from CISH, TGFBR2, FAS, CD7, CBLB, KLRC1, and CD38.

In some embodiments, the present disclosure provides a kit comprising a first gene editing system targeting the CISH gene, and a second gene editing system targeting a gene selected from TGFBR2, FAS, CD7, CBLB, KLRC1, and CD38.

In some embodiments, the present disclosure provides a kit comprising a first gene editing system targeting the TGFBR2 gene, and a second gene editing system targeting a gene selected from FAS, CD7, CBLB, KLRC1, and CD38.

In some embodiments, the present disclosure provides a kit comprising a first gene editing system targeting the FAS gene, and a second gene editing system targeting a gene selected from CD7, CBLB, KLRC1, and CD38.

In some embodiments, the present disclosure provides a kit comprising a first gene editing system targeting the CD7 gene, and a second gene editing system targeting a gene selected from CBLB and KLRC1.

In some embodiments, the present disclosure provides a kit comprising a first gene editing system targeting the CBLB gene, and a second gene editing system targeting a gene selected from KLRC1 and CD38.

In some embodiments, the present disclosure provides a kit comprising a first gene editing system targeting the KLRC1 gene, and a second gene editing system targeting CD38.

In some embodiments, the present disclosure provides a kit comprising a first gene editing system targeting the KLRC1 gene, and a second gene editing system targeting CD38.

In some embodiments, the present disclosure provides a kit comprising a first gene editing system targeting the KLRC1 gene, and a second gene editing system targeting a gene selected from PD1, TGFBR2, CISH, CD38, CBLB, TIGIT, TIM-3, LAG3, FAS, and TGFBR2.

In another aspect, the present disclosure provides a method for reducing immunogenicity of a cell comprising introducing into the cell one or more of the gene editing systems disclosed herein.

In some embodiments, the cell is an allogenic cell. In some embodiments, the cell is an immune cell. In some embodiments, the immune cell is a T cell, B cell, natural killer cell (NK cell), macrophage, dendritic cell, monocyte, granulocyte, or mast cell. In some embodiments, the as immune cell comprises a chimeric antigen receptor. In some embodiments, the cell is a T cell. In some embodiments, the T cell comprises a chimeric antigen receptor. In some embodiments, the T cell is a CAR-T cell. In some embodiments, the cell is a NK cell. In some embodiments, the NK cell comprises a chimeric antigen receptor. In some embodiments, the NK cell is a CAR-NK cell. In some embodiments, the cell is differentiated from a pluripotent stem cell. In some embodiments, the cell is differentiated from an iPSC or an ESC. In some embodiments, the cell is a primary cell.

In another aspect, the present disclosure provides a method for reducing graft versus host (GvH) response involved in administering allogenic cell into a subject, comprising reducing immunogenicity of the allogenic cell by introducing into the allogenic cell any one or more of the gene editing systems disclosed herein.

In some embodiments, the allogeneic cell is an immune cell. In some embodiments, the immune cell is a T cell, B cell, natural killer cell (NK cell), macrophage, dendritic cell, monocyte, granulocyte, or mast cell. In some embodiments, the immune cell comprises a chimeric antigen receptor. In some embodiments, the allogeneic cell is a T cell. In some embodiments, the T cell comprises a chimeric antigen receptor. In some embodiments, the T cell is a CAR-T cell. In some embodiments, the allogeneic cell is a NK cell. In some embodiments, the NK cell comprises a chimeric antigen receptor. In some embodiments, the NK cell is a CAR-NK cell. In some embodiments, the cell is differentiated from a pluripotent stem cell. In some embodiments, the cell is differentiated from an iPSC or an ESC. In some embodiments, the cell is a primary cell.

### BRIEF DESCRIPTION OF THE FIGURES

FIG. 1 illustrates exemplary base editors that can be used in the gene editing systems disclosed herein. The various versions of base editors are denoted as V1, V2, V3, V4, and V5, with constructs denoted as tBE-V1-rA1, tBE-V2-rA1, tBE-V3-rA1, tBE-V4-rA1, tBE-V5-rA1, and tBE-V5-mA3. FIG. 1A shows schematic diagrams illustrating the construction and development of various versions of base editors. FIG. 1B shows interactions of molecular components in different versions of the base editors. Base editors of V2 to V5 illustrate different strategies to cleave mA3dCDI off. The dCDI domain could be cleaved off from APOBEC through a two-component interaction of the TEV site and a free TEV protease (V2), a N22p-fused TEV protease (V3), or a TEV protease reconstituted by an mgRNA-boxB (V4). In the version 5 (V5) of the base editor, the dCDI is cleaved off from APOBEC through a three-component interaction of TEV site, TEVn, and N22p-TEVc.

FIG. 2 represents editing efficiencies induced by tBE with the pairs of mgRNA and its hgRNAs targeting human TRAC. FIG. 2A is a schematic diagram illustrating the co-transfection of mgRNAs and its different hgRNAs for human TRAC gene with tBE-V5-mA3 (SEQ ID NO: 731) and nCas9 (SEQ ID NO: 732). FIG. 2B shows the editing efficiency induced by tBE-V5-mA3 with indicated pairs of mgRNA/hgRNA at indicated sites. FIG. 2C shows the editing frequency for each mgRNA/hgRNA pair targeting human TRAC gene calculated by EditR analysis.

FIG. 3 is the verification of TRAC knock out (KO) on protein level. FIG. 3A-C are results of flow analysis of surface CD3 level for MOCK (A) or cells transfected with tBE+TRAC-mg2-U1 (B) or tBE+TRAC-mg4-U2 (C) in Jurkat T cells (CD3 form TCR-CD3 complex with TCR). FIG. 3D is a summary of CD3+ cell ratio for FIG. 3A-C. FIG. 3E-G are results of flow analysis of surface CD3 level for MOCK (E) or cells transfected with tBE+TRAC-mg2-U1 (F) or tBE+TRAC-mg4-U2 (G) in primary T cells. FIG. 3H is a summary of CD3+ cell ratio for 2E-G.

FIG. 4 represents editing efficiencies induced by tBE with the pairs of mgRNA and its hgRNAs targeting human CD52. FIG. 4A is a schematic diagram illustrating the co-transfection of mgRNAs and its different hgRNAs for human CD52 gene with tBE-V5-mA3 (SEQ ID NO: 731) and nCas9 (SEQ ID NO: 732). FIG. 4B shows the editing efficiency induced by tBE-V5-mA3 with indicated pairs of mgRNA/hgRNA at indicated sites. FIG. 4C shows the editing frequency for each mgRNA/hgRNA pair targeting human CD52 gene calculated by EditR analysis.

FIG. 5 is the verification of CD52 KO or TRAC/CD52 double KO on protein level. FIG. 5A-C are results of flow analysis of surface CD3 level and CD52 protein level for MOCK (A) or cells transfected with tBE+TRAC-mg2-U1+CD52-mg2-U3 (B) or tBE+TRAC-mg2-U1+CD52-mg3-U2 (C) in Jurkat T cells. FIG. 5D is a summary of CD3+ or CD52+ cell ratio for 5A-C. FIG. 5E-F are results of flow analysis of CD52 protein level for MOCK (E) or cells transfected with tBE+CD52-mg2-U3 (F) in primary T cells. FIG. 5G is a summary of CD52+ cell ratio for 5E-F. FIG. 5H-J are results of flow analysis of surface CD3 level and CD52 protein level for MOCK (H) or cells transfected with tBE+TRAC-mg2-U1+CD52-mg2-U3 (I) or tBE+TRAC-mg4-U2+CD52-mg2-U3 (J) in primary T cells. FIG. 5K is a summary of CD3+ or CD52+ cell ratio for 4H-J.

FIG. 6 represents editing efficiencies induced by tBE with the pairs of mgRNA and its hgRNAs targeting human B2M. FIG. 6A is a schematic diagram illustrating the co-transfection of mgRNAs and its different hgRNAs for human B2M gene with tBE-V5-mA3 (SEQ ID NO: 731) and nCas9 (SEQ ID NO: 732). FIG. 6B shows the editing efficiency induced by tBE-V5-mA3 with indicated pairs of mgRNA/hgRNA at indicated sites. FIG. 6C shows the editing frequency for each mgRNA/hgRNA pair targeting human B2M gene calculated by EditR analysis.

FIG. 7 is the verification of B2M KO or TRAC/B2M double KO on protein level. FIG. 7 A-C are results of flow analysis of B2M protein level for MOCK (A) or cells transfected with tBE+B2M-mg1-U3 (B) or tBE+B2M-mg2-U1 (C) in Jurkat T cells. FIG. 7 D is a summary of B2M+ cell ratio for 7A-C. FIG. 7 E-G are results of flow analysis of B2M and TRAC protein level for MOCK (E) or cells transfected with tBE+B2M-mg1-U3 (F) or tBE+B2M-mg1-U3+TRAC-mg4-U2 (G) in primary T cells. FIG. 7 H is a summary of TRAC+ or B2M+ cell ratio for 7E-G.

FIG. 8 represents editing efficiencies induced by tBE with the pairs of mgRNA and its hgRNAs targeting human PDCD1. FIG. 8A is a schematic diagram illustrating the co-transfection of mgRNAs and its different hgRNAs for human PDCD1 gene with tBE-V5-mA3 (SEQ ID NO: 731) and nCas9 (SEQ ID NO: 732). FIG. 8B shows the editing efficiency induced by tBE-V5-mA3 with indicated pairs of mgRNA/hgRNA at indicated sites. FIG. 8C shows the editing frequency for each mgRNA/hgRNA pair targeting human PDCD1 gene calculated by EditR analysis.

FIG. 9 is the verification of PDCD1 KO or TRAC/CD52/PDCD1 triple KO on protein as level. FIG. 9A-D are results of flow analysis of PD1 protein level for MOCK (A) or cells transfected with tBE+PDCD1-mg6-U2 (B), tBE+PDCD1-mg7-U2 (C) or PDCD1-mg15-U1 in primary T cells. FIG. 9E is a summary of PD1+ cell ratio for 9A-D. FIG. 9F-M are results of flow analysis of surface CD3 level and CD52 protein level (F-I) and PD1 protein level (J-M) for MOCK (F, J) or cells transfected with tBE+TRAC-mg4-U2 (G, K), tBE+TRAC-mg4-U2+CD52-mg2-U3 (H, L) or tBE+TRAC-mg4-U2+CD52-mg2-U3+PDCD1-mg7-U2 (I, M) in primary T cells. FIG. 9N is a summary of CD3+, CD52+ or PD1+ cell ratio for 9F-M.

FIG. 10 represents editing efficiencies induced by tBE with the pairs of mgRNA and its hgRNAs targeting human CTLA4. FIG. 10A is a schematic diagram illustrating the co-transfection of mgRNAs and its different hgRNAs for human CTLA4 gene with tBE-V5-mA3 (SEQ ID NO: 731) and nCas9 (SEQ ID NO: 732). FIG. 10B shows the editing efficiency induced by tBE-V5-mA3 with indicated pairs of mgRNA/hgRNA at indicated sites. FIG. 10C shows the editing frequency for each mgRNA/hgRNA pair targeting human CTLA4 gene calculated by EditR analysis.

FIG. 11 represents editing efficiencies induced by tBE with the pairs of mgRNA and its hgRNAs targeting human TIGIT. FIG. 11A is a schematic diagram illustrating the co-transfection of mgRNAs and its different hgRNAs for human TIGIT gene with tBE-V5-mA3 (SEQ ID NO: 731) and nCas9 (SEQ ID NO: 732). FIG. 1I B shows the editing efficiency induced by tBE-V5-mA3 with indicated pairs of mgRNA/hgRNA at indicated sites. FIG. 11C shows the editing frequency for each mgRNA/hgRNA pair targeting human TIGIT gene calculated by EditR analysis.

FIG. 12 represents editing efficiencies induced by tBE with the pairs of mgRNA and its hgRNAs targeting human TIM3. FIG. 12A is a schematic diagram illustrating the co-transfection of mgRNAs and its different hgRNAs for human TIM3 gene with tBE-V5-mA3 (SEQ ID NO: 731) and nCas9 (SEQ ID NO: 732). FIG. 12B shows the editing efficiency induced by tBE-V5-mA3 with indicated pairs of mgRNA/hgRNA at indicated sites. FIG. 12C shows the editing frequency for each mgRNA/hgRNA pair targeting human TIM3 gene calculated by EditR analysis.

FIG. 13 represents editing efficiencies induced by tBE with the pairs of mgRNA and its hgRNAs targeting human LAG3. FIG. 13A is a schematic diagram illustrating the co-transfection of mgRNAs and its different hgRNAs for human LAG3 gene with tBE-V5-mA3 (SEQ ID NO: 731) and nCas9 (SEQ ID NO: 732). FIG. 13B shows the editing efficiency induced by tBE-V5-mA3 with indicated pairs of mgRNA/hgRNA at indicated sites. FIG. 13C shows the editing frequency for each mgRNA/hgRNA pair targeting human LAG3 gene calculated by EditR analysis.

FIG. 14 represents editing efficiencies induced by tBE with the pairs of mgRNA and its hgRNAs targeting human CISH. FIG. 14A is a schematic diagram illustrating the co-transfection of mgRNAs and its different hgRNAs for human CISH gene with tBE-V5-mA3 (SEQ ID NO: 731) and nCas9 (SEQ ID NO: 732). FIG. 14B shows the editing efficiency induced by tBE-V5-mA3 with indicated pairs of mgRNA/hgRNA at indicated sites. FIG. 14C shows the editing frequency for each mgRNA/hgRNA pair targeting human CISH gene calculated by EditR analysis.

FIG. 15 is the verification of CISH KO on protein level. FIG. 15A-C are results of flow as analysis of CISH protein level for MOCK (A) or cells transfected with tBE+CISH-mg2-U2 (B) or tBE+CISH-mg3-U3 (C) in K562 cells. FIG. 15D is a summary of CISH+ cell ratio for 15A-C.

FIG. 16 represents editing efficiencies induced by tBE with the pairs of mgRNA and its hgRNAs targeting human TGFBR2. FIG. 16A is a schematic diagram illustrating the co-transfection of mgRNAs and its different hgRNAs for human TGFBR2 gene with tBE-V5-mA3 (SEQ ID NO: 731) and nCas9 (SEQ ID NO: 732). FIG. 16B shows the editing efficiency induced by tBE-V5-mA3 with indicated pairs of mgRNA/hgRNA at indicated sites. FIG. 16C shows the editing frequency for each mgRNA/hgRNA pair targeting human TGFBR2 gene calculated by EditR analysis.

FIG. 17 is the verification of TGFBR2 KO on protein level. FIG. 17A-C are results of flow analysis of TGFBR2 protein level for MOCK (A) or cells transfected with tBE+TGFBR2-mg3-U1 (B) or tBE+TGFBR2-mg4-U1 (C) in NK92 cells. FIG. 17D is a summary of TGFBR2+cell ratio for 17A-C.

FIG. 18 represents editing efficiencies induced by tBE with the pairs of mgRNA and its hgRNAs targeting human FAS. FIG. 18A is a schematic diagram illustrating the co-transfection of mgRNAs and its different hgRNAs for human FAS gene with tBE-V5-mA3 (SEQ ID NO: 731) and nCas9 (SEQ ID NO: 732). FIG. 18B shows the editing efficiency induced by tBE-V5-mA3 with indicated pairs of mgRNA/hgRNA at indicated sites. FIG. 18C shows the editing frequency for each mgRNA/hgRNA pair targeting human FAS gene calculated by EditR analysis.

FIG. 19 is the verification of FAS KO on protein level. FIG. 19A-C are results of flow analysis of FAS protein level for MOCK (A) or cells transfected with tBE+FAS-mg1-U1 (B) or tBE+FAS-mg5-U1 (C) in Jurkat cells. FIG. 19D is a summary of FAS+ cell ratio for 19A-C.

FIG. 20 represents editing efficiencies induced by tBE with the pairs of mgRNA and its hgRNAs targeting human CD7. FIG. 20A is a schematic diagram illustrating the co-transfection of mgRNAs and its different hgRNAs for human CD7 gene with tBE-V5-mA3 (SEQ ID NO: 731) and nCas9 (SEQ ID NO: 732). FIG. 20B shows the editing efficiency induced by tBE-V5-mA3 with indicated pairs of mgRNA/hgRNA at indicated sites. FIG. 20C shows the editing frequency for each mgRNA/hgRNA pair targeting human CD7 gene calculated by EditR analysis.

FIG. 21 represents editing efficiencies induced by tBE with the pairs of mgRNA and its hgRNAs targeting human CBLB. FIG. 21A is a schematic diagram illustrating the co-transfection of mgRNAs and its different hgRNAs for human CBLB gene with tBE-V5-mA3 (SEQ ID NO: 731) and nCas9 (SEQ ID NO: 732). FIG. 21B shows the editing efficiency induced by tBE-V5-mA3 with indicated pairs of mgRNA/hgRNA at indicated sites. FIG. 21C shows the editing efficiency for each mgRNA/hgRNA pairs targeting human CBLB gene calculated by EditR analysis.

FIG. 22 is the verification of CBLB KO on protein level. FIG. 22 is the result of western Blot analysis of CBLB protein level for MOCK (NC) or cells transfected with tBE+CBLB-mg2-U2 or tBE+CBLB-mg10-U1 in NK92 cells.

FIG. 23 represents editing efficiencies induced by tBE with the pairs of mgRNA and its hgRNAs targeting human KLRC1. FIG. 23A is a schematic diagram illustrating the co-transfection as of mgRNAs and its different hgRNAs for human KLRC1 gene with tBE-V5-mA3 (SEQ ID NO: 731) and nCas9 (SEQ ID NO: 732). FIG. 23B shows the editing efficiency induced by tBE-V5-mA3 with indicated pairs of mgRNA/hgRNA at indicated sites. FIG. 23C shows the editing efficiency for mgRNA/hgRNA pairs targeting human KLRC1 gene calculated by EditR analysis.

FIG. 24 is the verification of KLRC1 KO on protein level. FIG. 24 A-C are results of flow analysis of NKG2A protein level for MOCK (A) or cells transfected with tBE+KLRC1-mg2-U1 (B) or tBE+KLRC1-mg5-U1 (C) in NK92 cells. FIG. 24 D is a summary of NKG2A+ cell ratio for 24A-C.

FIG. 25 represents editing efficiencies induced by tBE with the pairs of mgRNA and its hgRNAs targeting human CD38. FIG. 25A is a schematic diagram illustrating the co-transfection of mgRNAs and its different hgRNAs for human CD38 gene with tBE-V5-mA3 (SEQ ID NO: 731) and nCas9 (SEQ ID NO: 732). FIG. 25B shows the editing efficiency induced by tBE-V5-mA3 with indicated pairs of mgRNA/hgRNA at indicated sites. FIG. 25C shows the editing efficiency for mgRNA/hgRNA pairs targeting human CD38 gene calculated by EditR analysis.

FIG. 26 is the verification of CD38 KO on protein level. FIG. 26A-C are results of flow analysis of CD38 protein level for MOCK (A) or cells transfected with tBE+CD38-mg2-U2 (B) or tBE+CD38-mg7-U1 (C) in NK92 cells. FIG. 25D is a summary of NKG2A+ cell ratio for 25A-C.

### DETAILED DESCRIPTION

### Definitions

In the present disclosure, unless otherwise specified, the scientific and technical terms used herein have the meanings generally understood by a person skilled in the art. Although any methods and materials similar or equivalent to those described herein find use in the practice of the present disclosure, the preferred methods and materials are described herein. Accordingly, the terms defined herein are more fully described by reference to the Specification as a whole.

As used herein, the singular terms “a,” “an,” and “the” include the plural reference unless the context clearly indicates otherwise.

As used herein, “and/or” refers to and encompasses any and all possible combinations of one or more of the associated listed items, as well as the lack of combinations when interpreted in the alternative (“or”). Moreover, the present invention also contemplates that in some embodiments of the invention, any feature or combination of features set forth herein can be excluded or omitted.

Unless the context requires otherwise, the terms “comprise,” “comprises,” and “comprising,” or similar terms are intended to mean a non-exclusive inclusion, such that a recited list of elements or features does not include those stated or listed elements solely, but may include other elements or features that are not listed or stated.

Unless otherwise indicated, nucleic acids are written left to right in the 5′ to 3′ orientation, and amino acid sequences are written left to right in amino to carboxy orientation, respectively. A number “n”, when used in the context of an amino acid sequence, refers to the n^{th} amino acid in the amino acid sequence counting from the amino end. For example, “amino acid 15” refers to the 15^{th} amino acid in a certain amino acid sequence. For example, “R15” refers to the 15^{th} amino acid, which is an arginine (R), in a certain amino acid sequence.

It is to be understood that this disclosure is not limited to the particular methodology, protocols, and reagents described, as these may vary, depending upon the context in which they are used by those skilled in the art.

As used herein, the terms “percent identity” and “% identity,” as applied to nucleic acid or polynucleotide sequences, refer to the percentage of residue matches between at least two nucleic acid or polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences.

Percent identity between nucleic acid or polynucleotide sequences may be determined using a suite of commonly used and freely available sequence comparison algorithms provided by the National Center for Biotechnology Information (NCBI) Basic Local Alignment Search Tool (BLAST) (Altschul, S. F. et al. (1990) J. Mol. Biol. 215:403-410), which is available from several sources, including the NCBI, Bethesda, Md., and on the Internet at http://www.ncbi.nlm.nih.gov/BLAST/.

Nucleic acid or polynucleotide sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein. Specifically, degenerate codon substitutions may be achieved by generating sequences in which the third position of one or more selected (or all) codons is substituted with mixed-base and/or deoxyinosine residues (Batzer et al. (1991) Nucleic Acid Res 19:5081; Ohtsuka et al. (1985) J Biol Chem 260:2605-2608; Cassol et al. (1992); Rossolini et al. (1994) Mol Cell Probes 8:91-98). The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. The term nucleic acid is used interchangeably with polynucleotide, and (in appropriate contexts) gene, cDNA, and mRNA encoded by a gene.

As used herein, “percent (%) amino acid sequence identity” with respect to a peptide, polypeptide or protein sequence is defined as the percentage of amino acid residues in a candidate sequence that are identical with the amino acid residues in another peptide or polypeptide sequence, as after aligning the sequences and introducing gaps, if necessary, to achieve the maximum percent sequence identity, and not considering any conservative substitutions as part of the sequence identity. Percent amino acid sequence identity in the current disclosure is measured using BLAST software. Those skilled in the art can determine appropriate parameters for measuring alignment, including any algorithms needed to achieve maximal alignment over the full length of the sequences being compared.

An amino acid substitution refers to the replacement of one amino acid in a polypeptide with another amino acid. Amino acid substitutions can be conservative or non-conservative substitutions. Exemplary substitutions are shown in Table 1. Amino acid substitutions may be introduced into a protein of interest and the products screened for a desired activity, for example, retained/improved biological activity.

TABLE 1

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

Amino acids may be grouped according to common side-chain properties:
(1) hydrophobic: Norleucine, Met, Aia, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, “peptides,” “protein”, or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

As used herein, the term “encode” or “encoding” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

A “guide RNA” (gRNA) refers to a synthetic or expressed RNA sequence that comprises a CRISPR binding motif and a spacer. In some embodiments, the guide RNA is a single guide RNA. In some embodiments, the guide RNA is a dual-RNA structure. In some embodiments, the guide RNA is a dual-RNA structure formed by a ligand-bound CRISPR RNA (crRNA) and a trans-activating crRNA (tracrRNA). In some embodiments, the guide RNA is a LigoRNA. A “spacer” is a DNA-targeting motif, which is a sequence that is complementary to a target specific DNA region. In some embodiments, the guide RNA is a crRNA-tracrRNA dual RNA structure, and the crRNA comprises the spacer. The CRISPR binding motif of a guide RNA can bind to a Cas enzyme and DNA-targeting motif of the gRNA can guide the complex to a specific target location on a DNA. In some embodiments, the guide RNA is a crRNA-tracrRNA dual RNA structure, and the base-pair structure formed by the crRNA and the tracrRNA comprises the CRISPR binding motif. A guide RNA may further comprise one or more protein-binding motifs.

As used herein, a “fusion protein” is a protein comprising at least two domains that are encoded by separate genes that have been joined a single polypeptide. For example, a fusion protein can comprise two domains that are encoded by separate genes that have been joined so that they are transcribed and translated as a single unit, producing a single polypeptide. In some embodiments, the at least two domains are fused together directly. In some embodiments, the domains are connected by one or more linkers.

The term “genetic modification” and its grammatical equivalents as used herein can refer to one or more alterations of a nucleic acid, e.g., the nucleic acid within an organism's genome. For example, genetic modification can refer to alterations, additions, and/or deletion of genes or portions of genes or other nucleic acid sequences. A genetically modified cell can also refer to a cell with an added, deleted, and/or altered gene or portion of a gene. A genetically modified cell can also refer to a cell with an added nucleic acid sequence that is not a gene or gene portion. Genetic modifications include, for example, both transient knock-in or knock-down mechanisms, and mechanisms that result in permanent knock-in, knock-down, or knock-out of target genes or portions of genes or nucleic acid sequences. Genetic modifications include, for example, both transient knock-in and mechanisms that result in permanent knock-in of nucleic acids sequences. Genetic modifications also include, for example, reduced or increased transcription, reduced or increased mRNA stability, reduced or increased translation, and reduced or increased protein stability.

As used herein, a composition refers to any mixture of two or more products, substances, or compounds, including cells.

The term “subject” means any animal such as a mammal, e.g., a human.

As used herein, the term “treat,” “treating,” or “treatment” refers to ameliorating a disease or disorder, e.g., slowing or arresting or reducing the development of the disease or disorder or reducing at least one of the clinical symptoms thereof. For example, in some embodiments, ameliorating a disease or disorder can include obtaining a beneficial or desired clinical result that includes, but is not limited to, any one or more of: alleviation of one or more symptoms, diminishment of extent of disease, preventing or delaying spread of disease, preventing or delaying recurrence of disease, delay or slowing of disease progression, amelioration of the disease state, inhibiting or eliminating the disease or progression of the disease, inhibiting or slowing the disease or its progression, arresting its development, and remission (whether partial or total).

As used herein, for a given subject, “allogeneic” cells refer to cells obtained from different individuals of the same species with the subject, and are genetically dissimilar with the cells obtained from the given subject.

As used herein, the term “immunogenicity” refers to the ability or tendency of a substance to prove an unwanted immune response against itself in a subject.

### Immunogenicity of Allogeneic Cells

Allogeneic cell therapy often faces two major challenges. First, the administered allogeneic cells may cause life-threatening graft-versus-host disease (GvHD). Second, these as allogeneic cells may be rapidly eliminated by the host immune system, limiting their persistence of bioactivity.

Graft-versus-host disease (GvHD) is a systemic disorder that occurs when the graft's immune cells recognize the host as foreign and attack the recipient's body cells. “Graft” refers to transplanted, or donated tissue, and “host” refers to the tissues of the recipient. GvHD is one of the main causes of death after allogeneic hematopoietic stem cell transplantation, so it must be prevented. Because T-cell alloreactivity is dependent on the interaction of T-cell receptors (TCRs) with alloantigens presented by human leukocyte antigens (HLAs), TCR-depleted T cells do not cause GvH responses when infused into HLA-unmatched patients. Many groups are working to reduce the risk of GvHD by genetic ablation of the TCR locus, mainly the TCRα constant (TRAC, human TRAC: ENMG00000277734), which can be effective to reduce the risk of GvHD and reduce the GvH responses involved in administering allogenic cell into a subject.

On the other hand, to prevent or reduce host immune rejection, one way is to reduce the immunogenicity of the allogenic T cells, such as by genetic abrogation of the b2-microglobulin (B2M) gene (human B2M: ENMG00000166710) to disrupt the MHC class I molecules. Another way is to delete the CD52 (human CD52: ENMG00000169442) of the donor T cells and use anti-CD52 monoclonal antibody to eliminate host T cells (which express CD52) to avoid allorejection. Besides, inhibitory checkpoints (e.g., PD-1, coded by PDCD1 gene, human PDCD1: ENMG00000188389) can be knocked out separately or simultaneously with TRAC, CD52, B2M, PDCD1, CTLA4, TIGIT, TIM3, LAG3, CISH, TGFBR2, FAS, CD7, CBLB, KLRC1, and/or CD38 to enhance the efficacy and persistence of the autologous or allogenic CAR-T cells or NK cells. (Depil, S., et al. “‘Off-the-shelf’allogeneic CAR T cells: development and challenges.” Nature reviews Drug discovery 19.3 (2020): 185-199; Jung, In-Young, and Jungmin Lee. “Unleashing the therapeutic potential of CAR-T cell therapy using gene-editing technologies.” Molecules and Cells 41.8 (2018): 717; Lin, Haolong, et al. “Advances in universal CAR-T cell therapy.” Frontiers in Immunology (2021): 4014). CD7 or CD38 could be knocked out for anti-CD7 or anti-CD38 CAR-T cells to prevent fratricide. FAS/FASL signal induces apoptosis of cytotoxic T cells, which dampens the anti-tumor efficacy of CAR-T therapy. TGF-β secreted in the tumor microenvironment (TME) suppresses T cell function by binding to TGFBR2. Cytokine-induced SH2 (CISH) protein is induced in CD8+ T-cells upon TCR stimulation and inhibits T-cell anti-tumor function. CISH is also a key negative regulator of IL-15 signaling in NK cells. CBLB has been characterized as an intracellular checkpoint in T cells and also in NK cells and deletion of CBLB enhances the function of T and NK cells. KLRC1 gene encodes the NK cell inhibitory receptor NKG2A, which is a potent NK cell immune checkpoint.

The present disclosure provides that disrupting expression (for example, by knocking down or knocking out) of the following genes, either separately or in combination, can reduce or prevent host immune rejection in a subject, or promote the survival and persistence of the therapeutic cells. The genes are the TCRα constant gene (TRAC, human TRAC: ENMG00000277734), the b2-microglobulin (B2M) gene (human B2M: ENMG00000166710), as the CD52 gene (human CD52: ENMG00000169442), the PDCD1 gene (human PDCD1: ENMG00000188389), the cytotoxic T-lymphocyte associated protein 4 gene (CTLA4, ENMG00000163599), the T cell immunoreceptor with Ig and ITIM domains gene (TIGIT, ENMG00000181847), the hepatitis A virus cellular receptor 2 gene (HAVCR2/TIM3, ENMG00000135077), the lymphocyte activating 3 gene (LAG3, ENMG00000089692), the cytokine inducible SH2 containing protein gene (CISH, ENMG00000114737), the transforming growth factor beta receptor 2 gene (TGFBR2, ENMG00000163513), and the Fas cell surface death receptor gene (FAS, ENMG00000026103), the CD7 gene (ENMG00000173762), the Cb1 proto-oncogene B (CBLB) gene (human CBLB: ENSG00000114423), the killer cell lectin like receptor C1 (KLRC1) gene (human KLRC1: ENSG00000134545), and the CD38 gene (human CD38: ENSG00000004468).

### Gene Editing Systems

The safety and efficiency of gene editing tools are of great importance in clinical applications. Previous studies have reported that the DSBs induced by Cas9 nuclease can activate a p53-mediated DDR pathway and then lead to cell death. Moreover, APOBEC/AID family members can trigger C-to-T base substitutions in single-stranded DNA (ssDNA) regions, which are formed randomly during various cellular processes including DNA replication, repair, and transcription. Thus, the specificity of previous base editing systems is compromised, limiting the applications of base editors (BEs) for therapeutic purposes.

In the present disclosure, a newly developed base editing system, transformer base editor (tBE), is used. tBE can specifically edit cytosine in target regions with no observable off-target mutations.

In some embodiments, the transformer base editor (tBE) system contains a deoxycytidine deaminase inhibitor (dCDI) domain and a split-TEV protease. Thus, tBE remains inactive at off-target sites with a cleavable fusion of dCDI domain and eliminates unintended off-target mutations. Only when binding at on-target sites, tBE is transformed to cleave off the dCDI domain and catalyzes targeted deamination for precise editing. Specifically, tBE uses one mgRNA (normally 20 nt) to bind at the target genomic site and one helper mgRNA (hgRNA, normally 10 to 20 nt) to bind at a nearby region (preferably upstream to the target genomic site). The binding of the two gRNAs can guide the components of tBE system to correctly assemble at the target genomic site for base editing. tBE can specifically edit cytosines in target regions with no observable off-target mutations, e.g., inducing a premature stop codon to repress target protein expression or destroying the GU-AG consensus sequences to disrupt splicing site. Furthermore, the tBE system, when using Cas9 nickase (D10A), is less toxic to cells than Cas9 nuclease as Cas9 nickase activates a lower level of p53-mediated DDR.

The present disclosure provides that tBE system can be used to disrupt TRAC, B2M, CD52, PDCD1, CTLA4, TIGIT, TIM3, LAG3, CISH, TGFBR2, FAS, CD7, CBLB, KLRC1, or CD38 gene in cells either separately or in combination, to prevent GvHD or to reduce immunogenicity of the cell, thus enhancing the expansion and persistence of CAR-T cells or other therapeutic cells after infusion. In some embodiments, the cells are human immune cells, such as human T cells and Natural Killer (NK) cells.

In some embodiments, tBE is used for genetic engineering in human T cells, NK cells, and other immune cells to construct allogenic or enhanced autologous Chimeric antigen receptor T (CAR-T) cells and other cell therapy products in clinical applications. In some embodiments, tBE is used to induce efficient and precise gene editing at genomic sites for disrupting genes related with graft versus host disease (GvHD), allorejection by the host, immune suppression, or T cell fratricide.

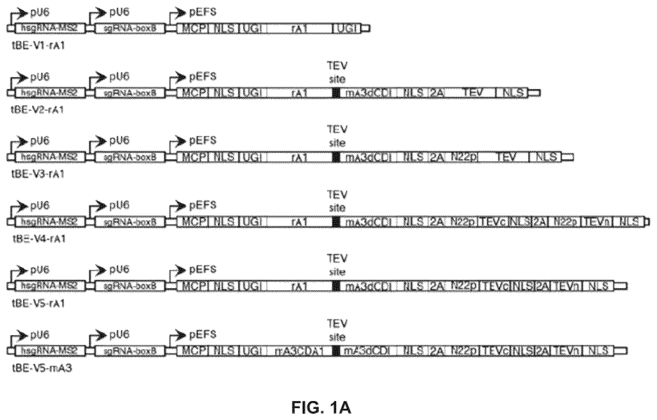

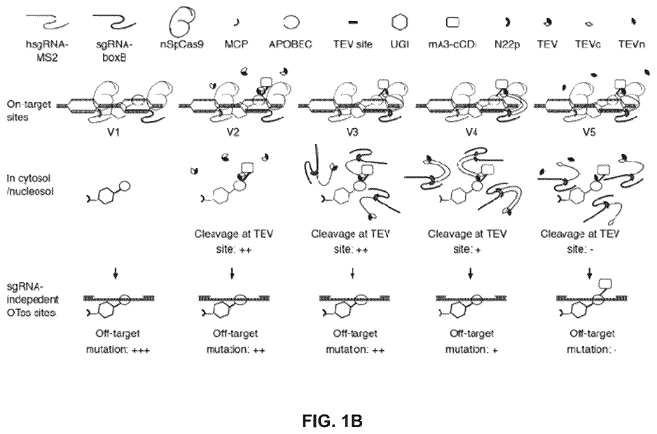

In some embodiments, a highly specific base editing system, transformer base editor (tBE), is used, which can edit cytosine in target regions with no observable off-target mutations. In some embodiments, the tBE is any one of the base editors described in WO2020156575A1, incorporated herein by reference in its entirety. For instance, the tBE can be any base editor as illustrated in FIG. 1.

The present disclosure provides multiple combinations of guide RNA (mgRNA) and helper mgRNA (hgRNA) with high editing efficiency for target genes: the T cell receptor alpha constant (TRAC) gene, the beta-2-microglobulin (B2M) gene, the CD52 gene, and the programmed cell death 1 (PDCD1) gene, the cytotoxic T-lymphocyte associated protein 4 (CTLA4) gene, the T cell immunoreceptor with Ig and ITIM domains (TIGIT) gene, the hepatitis A virus cellular receptor 2 (HAVCR2/TIM3) gene, the lymphocyte activating 3 (LAG3) gene, the cytokine inducible SH2 containing protein (CISH) gene, the transforming growth factor beta receptor 2 (TGFBR2) gene, the Fas cell surface death receptor (FAS) gene, the CD7 gene, the Cb1 proto-oncogene B (CBLB) gene, the killer cell lectin like receptor C1 (KLRC1) gene, and the CD38 gene respectively. In some embodiments, these pairs of mgRNA/hgRNA can be used for the construction of allogenic or enhanced autologous CAR-T cells, or other types of allogenic or enhanced cell therapies in clinical applications.

The base editors, combinations of mgRNA/hgRNA, and base editing methods provided herein can be applied to perform high-specificity and high-efficiency base editing in the genome of various eukaryotes. They achieve high specificity and efficiency at most sites. The present disclosure potentiates the clinical translation of tBE, especially in the construction of allogenic or enhanced autologous CAR-T cells and other types of allogenic or enhanced cell therapies.

In some embodiments, a base editor as used herein is a cytosine base editor (CBE), which comprises a combination of a CRISPR system and cytidine deaminase. A CBE effectuates a programmable cytosine to thymine (C-to-T) substitution. Because the base editing process does not depend on the generation of DNA double strand break (DSB), unwanted nucleotide insertions/deletions (indels) or DNA damage responses (DDRs) can be largely avoided.

In some embodiments, the gene editing system disclosed herein disrupts the targe gene by generating stop codons or destroy splicing sites in the target gene.

In some embodiments, the gene editing system disclosed herein induces C-to-T base editing in the codons of CAA (Gln), CAG (Gln), TGG (Trp, C-to-T on the opposite strand), or CGA (Arg) in the target gene to create a TAA, TAG, or TGA stop codon.

In some embodiments, the gene editing system disclosed herein induces G-to-A (C-to-T on the opposite strand) base editing in GT or AG splice site to destroy the GU-AG canonical as splicing pattern.

In some embodiments, present disclosure provides a gene editing system for disrupting the TRAC gene, wherein the gene editing system comprises a base editor and at least one guide RNA that is capable of binding to the TRAC gene. In some embodiments, a highly specific base editor, transformer base editor (tBE), is used to induce efficient and precise gene editing at genomic sites for disrupting the TRAC gene. A tBE comprises a combination of guide RNA (mgRNA) and helper mgRNA (hgRNA), wherein the mgRNA and hgRNA are capable of binding to the TRAC gene.

In some embodiments, present disclosure provides a gene editing system for disrupting the B2M gene, wherein the gene editing system comprises a base editor and at least one guide RNA that is capable of binding to the B2M gene. In some embodiments, a highly specific base editor, transformer base editor (tBE), is used to induce efficient and precise gene editing at genomic sites for disrupting the B2M gene. A tBE comprises a combination of guide RNA (mgRNA) and helper mgRNA (hgRNA), wherein the mgRNA and hgRNA are capable of binding to the B2M gene.

In some embodiments, present disclosure provides a gene editing system for disrupting the CD52 gene, wherein the gene editing system comprises a base editor and at least one guide RNA that is capable of binding to the CD52 gene. In some embodiments, a highly specific base editor, transformer base editor (tBE), is used to induce efficient and precise gene editing at genomic sites for disrupting the CD52 gene. A tBE comprises a combination of guide RNA (mgRNA) and helper mgRNA (hgRNA), wherein the mgRNA and hgRNA are capable of binding to the CD52 gene.

In some embodiments, present disclosure provides a gene editing system for disrupting the PDCD1 gene, wherein the gene editing system comprises a base editor and at least one guide RNA that is capable of binding to the PDCD1 gene. In some embodiments, a highly specific base editor, transformer base editor (tBE), is used to induce efficient and precise gene editing at genomic sites for disrupting the PDCD1 gene. A tBE comprises a combination of guide RNA (mgRNA) and helper mgRNA (hgRNA), wherein the mgRNA and hgRNA are capable of binding to the PDCD1 gene.

In some embodiments, present disclosure provides a gene editing system for disrupting the CTLA4 gene, wherein the gene editing system comprises a base editor and at least one guide RNA that is capable of binding to the CTLA4 gene. In some embodiments, a highly specific base editor, transformer base editor (tBE), is used to induce efficient and precise gene editing at genomic sites for disrupting the CTLA4 gene. A tBE comprises a combination of guide RNA (mgRNA) and helper mgRNA (hgRNA), wherein the mgRNA and hgRNA are capable of binding to the CTLA4 gene.

In some embodiments, present disclosure provides a gene editing system for disrupting the TIGIT gene, wherein the gene editing system comprises a base editor and at least one guide RNA that is capable of binding to the TIGIT gene. In some embodiments, a highly specific base editor, transformer base editor (tBE), is used to induce efficient and precise gene editing at as genomic sites for disrupting the TIGIT gene. A tBE comprises a combination of guide RNA (mgRNA) and helper mgRNA (hgRNA), wherein the mgRNA and hgRNA are capable of binding to the TIGIT gene.

In some embodiments, present disclosure provides a gene editing system for disrupting the TIM3 gene, wherein the gene editing system comprises a base editor and at least one guide RNA that is capable of binding to the TIM3 gene. In some embodiments, a highly specific base editor, transformer base editor (tBE), is used to induce efficient and precise gene editing at genomic sites for disrupting the TIM3 gene. A tBE comprises a combination of guide RNA (mgRNA) and helper mgRNA (hgRNA), wherein the mgRNA and hgRNA are capable of binding to the TIM3 gene.

In some embodiments, present disclosure provides a gene editing system for disrupting the LAG3 gene, wherein the gene editing system comprises a base editor and at least one guide RNA that is capable of binding to the LAG3 gene. In some embodiments, a highly specific base editor, transformer base editor (tBE), is used to induce efficient and precise gene editing at genomic sites for disrupting the LAG3 gene. A tBE comprises a combination of guide RNA (mgRNA) and helper mgRNA (hgRNA), wherein the mgRNA and hgRNA are capable of binding to the LAG3 gene.

In some embodiments, present disclosure provides a gene editing system for disrupting the CISH gene, wherein the gene editing system comprises a base editor and at least one guide RNA that is capable of binding to the CISH gene. In some embodiments, a highly specific base editor, transformer base editor (tBE), is used to induce efficient and precise gene editing at genomic sites for disrupting the CISH gene. A tBE comprises a combination of guide RNA (mgRNA) and helper mgRNA (hgRNA), wherein the mgRNA and hgRNA are capable of binding to the CISH gene.

In some embodiments, present disclosure provides a gene editing system for disrupting the TGFBR2 gene, wherein the gene editing system comprises a base editor and at least one guide RNA that is capable of binding to the TGFBR2 gene. In some embodiments, a highly specific base editor, transformer base editor (tBE), is used to induce efficient and precise gene editing at genomic sites for disrupting the TGFBR2 gene. A tBE comprises a combination of guide RNA (mgRNA) and helper mgRNA (hgRNA), wherein the mgRNA and hgRNA are capable of binding to the TGFBR2 gene.

In some embodiments, present disclosure provides a gene editing system for disrupting the FAS gene, wherein the gene editing system comprises a base editor and at least one guide RNA that is capable of binding to the FAS gene. In some embodiments, a highly specific base editor, transformer base editor (tBE), is used to induce efficient and precise gene editing at genomic sites for disrupting the FAS gene. A tBE comprises a combination of guide RNA (mgRNA) and helper mgRNA (hgRNA), wherein the mgRNA and hgRNA are capable of binding to the FAS gene.

In some embodiments, present disclosure provides a gene editing system for disrupting the CD7 gene, wherein the gene editing system comprises a base editor and at least one guide RNA that is capable of binding to the CD7 gene. In some embodiments, a highly specific base editor, as transformer base editor (tBE), is used to induce efficient and precise gene editing at genomic sites for disrupting the CD7 gene. A tBE comprises a combination of guide RNA (mgRNA) and helper mgRNA (hgRNA), wherein the mgRNA and hgRNA are capable of binding to the CD7 gene.

In some embodiments, the present disclosure provides a gene editing system for disrupting the CBLB gene, wherein the gene editing system comprises a base editor and at least one guide RNA that is capable of binding to the CBLB gene. In some embodiments, a highly specific base editor, transformer base editor (tBE), is used to induce efficient and precise gene editing at genomic sites for disrupting the CBLB gene. A tBE comprises a combination of guide RNA (mgRNA) and helper mgRNA (hgRNA), wherein the mgRNA and hgRNA are capable of binding to the CBLB gene.

In some embodiments, the present disclosure provides a gene editing system for disrupting the KLRC1 gene, wherein the gene editing system comprises a base editor and at least one guide RNA that is capable of binding to the KLRC1 gene. In some embodiments, a highly specific base editor, transformer base editor (tBE), is used to induce efficient and precise gene editing at genomic sites for disrupting the KLRC1 gene. A tBE comprises a combination of guide RNA (mgRNA) and helper mgRNA (hgRNA), wherein the mgRNA and hgRNA are capable of binding to the KLRC1 gene.

In some embodiments, the present disclosure provides a gene editing system for disrupting the CD38 gene, wherein the gene editing system comprises a base editor and at least one guide RNA that is capable of binding to the CD38 gene. In some embodiments, a highly specific base editor, transformer base editor (tBE), is used to induce efficient and precise gene editing at genomic sites for disrupting the CD38 gene. A tBE comprises a combination of guide RNA (mgRNA) and helper mgRNA (hgRNA), wherein the mgRNA and hgRNA are capable of binding to the CD38 gene.

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a T-cell receptor a constant (TRAC) gene and the hgRNA comprises a hgRNA spacer, wherein the nucleic acid sequence of the mgRNA spacer comprises a sequence selected from SEQ ID NOs: 1-5.

In some embodiments, the nucleic acid sequences of the mgRNA spacer and the hgRNA spacer comprise the sequences as set forth in Table 2.

TABLE 2
Combinations of mgRNA spacer and hgRNA spacer (TRAC)

| mgRNA | hgRNA |
|---|---|
| SEQ ID NO: 01 | SEQ ID NO: 39 |
| SEQ ID NO: 01 | SEQ ID NO: 40 |
| SEQ ID NO: 01 | SEQ ID NO: 41 |
| SEQ ID NO: 02 | SEQ ID NO: 39 |
| SEQ ID NO: 02 | SEQ ID NO: 40 |
| SEQ ID NO: 02 | SEQ ID NO: 41 |
| SEQ ID NO: 03 | SEQ ID NO: 42 |
| SEQ ID NO: 03 | SEQ ID NO: 43 |
| SEQ ID NO: 03 | SEQ ID NO: 44 |
| SEQ ID NO: 04 | SEQ ID NO: 45 |
| SEQ ID NO: 04 | SEQ ID NO: 46 |
| SEQ ID NO: 04 | SEQ ID NO: 47 |
| SEQ ID NO: 05 | SEQ ID NO: 48 |
| SEQ ID NO: 05 | SEQ ID NO: 49 |
| SEQ ID NO: 05 | SEQ ID NO: 50 |

In some embodiments, the nucleic acid sequences of the mgRNA and the hgRNA comprise respectively:

TABLE 26

| mgRNA | hgRNA |
|---|---|
| SEQ ID NO: 679 | SEQ ID NO: 680 |
| SEQ ID NO: 679 | SEQ ID NO: 681 |
| SEQ ID NO: 682 | SEQ ID NO: 684 |
| SEQ ID NO: 682 | SEQ ID NO: 685; or |
| SEQ ID NO: 683 | SEQ ID NO: 685 |

In another aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a CD52 gene and the hgRNA comprises a hgRNA spacer, wherein the nucleic acid sequence of the mgRNA spacer comprises a sequence selected from SEQ ID NOs: 6-8.

In some embodiments, the nucleic acid sequences of the mgRNA spacer and the hgRNA spacer comprise the sequences as set forth in Table 3.

TABLE 3
Combinations of mgRNA spacer and hgRNA spacer (CD52)

| mgRNA | hgRNA |
|---|---|
| SEQ ID NO: 06 | SEQ ID NO: 51 |
| SEQ ID NO: 06 | SEQ ID NO: 52 |
| SEQ ID NO: 06 | SEQ ID NO: 53 |
| SEQ ID NO: 07 | SEQ ID NO: 51 |
| SEQ ID NO: 07 | SEQ ID NO: 52 |
| SEQ ID NO: 07 | SEQ ID NO: 53 |
| SEQ ID NO: 08 | SEQ ID NO: 54 |
| SEQ ID NO: 08 | SEQ ID NO: 55 |
| SEQ ID NO: 08 | SEQ ID NO: 56 |

In some embodiments, the nucleic acid sequences of the mgRNA and the hgRNA comprise respectively:

TABLE 27

| mgRNA | hgRNA |
|---|---|
| SEQ ID NO: 686 | SEQ ID NO: 688 |
| SEQ ID NO: 687 | SEQ ID NO: 688; or |
| SEQ ID NO: 689 | SEQ ID NO: 690 |

In another aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a b2-microglobulin (B32M) gene and the hgRNA comprises a hgRNA spacer, wherein the nucleic acid sequence of the mgRNA spacer comprises a sequence selected from SEQ ID NOs: 9-19.

In some embodiments, the nucleic acid sequences of the mgRNA spacer and the hgRNA spacer comprise the sequences as set forth in Table 4.

TABLE 4
Combinations of mgRNA spacer and hgRNA spacer (B2M)

| mgRNA | hgRNA |
|---|---|
| SEQ ID NO: 09 | SEQ ID NO: 57 |
| SEQ ID NO: 09 | SEQ ID NO: 58 |
| SEQ ID NO: 09 | SEQ ID NO: 59 |
| SEQ ID NO: 09 | SEQ ID NO: 60 |
| SEQ ID NO: 09 | SEQ ID NO: 61 |
| SEQ ID NO: 10 | SEQ ID NO: 62 |
| SEQ ID NO: 11 | SEQ ID NO: 63 |
| SEQ ID NO: 11 | SEQ ID NO: 64 |
| SEQ ID NO: 11 | SEQ ID NO: 65 |
| SEQ ID NO: 11 | SEQ ID NO: 66 |
| SEQ ID NO: 12 | SEQ ID NO: 67 |
| SEQ ID NO: 12 | SEQ ID NO: 68 |
| SEQ ID NO: 12 | SEQ ID NO: 69 |
| SEQ ID NO: 12 | SEQ ID NO: 70 |
| SEQ ID NO: 12 | SEQ ID NO: 71 |
| SEQ ID NO: 13 | SEQ ID NO: 67 |
| SEQ ID NO: 13 | SEQ ID NO: 68 |
| SEQ ID NO: 13 | SEQ ID NO: 69 |
| SEQ ID NO: 13 | SEQ ID NO: 70 |
| SEQ ID NO: 13 | SEQ ID NO: 71 |
| SEQ ID NO: 14 | SEQ ID NO: 67 |
| SEQ ID NO: 14 | SEQ ID NO: 68 |
| SEQ ID NO: 14 | SEQ ID NO: 69 |
| SEQ ID NO: 14 | SEQ ID NO: 70 |
| SEQ ID NO: 14 | SEQ ID NO: 71 |
| SEQ ID NO: 15 | SEQ ID NO: 72 |
| SEQ ID NO: 15 | SEQ ID NO: 73 |
| SEQ ID NO: 15 | SEQ ID NO: 74 |
| SEQ ID NO: 15 | SEQ ID NO: 75 |
| SEQ ID NO: 15 | SEQ ID NO: 76 |
| SEQ ID NO: 16 | SEQ ID NO: 72 |
| SEQ ID NO: 16 | SEQ ID NO: 73 |
| SEQ ID NO: 16 | SEQ ID NO: 74 |
| SEQ ID NO: 16 | SEQ ID NO: 75 |
| SEQ ID NO: 16 | SEQ ID NO: 76 |
| SEQ ID NO: 17 | SEQ ID NO: 77 |
| SEQ ID NO: 17 | SEQ ID NO: 78 |
| SEQ ID NO: 17 | SEQ ID NO: 79 |
| SEQ ID NO: 17 | SEQ ID NO: 80 |
| SEQ ID NO: 18 | SEQ ID NO: 77 |
| SEQ ID NO: 18 | SEQ ID NO: 78 |
| SEQ ID NO: 18 | SEQ ID NO: 79 |
| SEQ ID NO: 18 | SEQ ID NO: 80 |
| SEQ ID NO: 19 | SEQ ID NO: 81 |
| SEQ ID NO: 19 | SEQ ID NO: 82 |

In some embodiments, the nucleic acid sequences of the mgRNA and the hgRNA comprise respectively:

TABLE 28

| mgRNA | hgRNA |
|---|---|
| SEQ ID NO: 697 | SEQ ID NO: 698; or |
| SEQ ID NO: 699 | SEQ ID NO: 700 |

In another aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a programmed cell death protein 1 (PDCD1) gene and the hgRNA comprises a hgRNA spacer, wherein the nucleic acid sequence of the mgRNA spacer comprises a sequence selected from SEQ ID NOs: 20-38.

In some embodiments, the nucleic acid sequences of the mgRNA spacer and the hgRNA spacer comprise the sequences as set forth in Table 5.

TABLE 5
Combinations of mgRNA spacer and hgRNA spacer (PDCD1)

| mgRNA | hgRNA |
|---|---|
| SEQ ID NO: 20 | SEQ ID NO: 83 |
| SEQ ID NO: 20 | SEQ ID NO: 84 |
| SEQ ID NO: 20 | SEQ ID NO: 85 |
| SEQ ID NO: 20 | SEQ ID NO: 86 |
| SEQ ID NO: 21 | SEQ ID NO: 87 |
| SEQ ID NO: 22 | SEQ ID NO: 87 |
| SEQ ID NO: 23 | SEQ ID NO: 88 |
| SEQ ID NO: 23 | SEQ ID NO: 89 |
| SEQ ID NO: 23 | SEQ ID NO: 90 |
| SEQ ID NO: 24 | SEQ ID NO: 91 |
| SEQ ID NO: 24 | SEQ ID NO: 92 |
| SEQ ID NO: 24 | SEQ ID NO: 93 |
| SEQ ID NO: 25 | SEQ ID NO: 94 |
| SEQ ID NO: 25 | SEQ ID NO: 95 |
| SEQ ID NO: 25 | SEQ ID NO: 96 |
| SEQ ID NO: 26 | SEQ ID NO: 97 |
| SEQ ID NO: 26 | SEQ ID NO: 98 |
| SEQ ID NO: 26 | SEQ ID NO: 96 |
| SEQ ID NO: 27 | SEQ ID NO: 99 |
| SEQ ID NO: 27 | SEQ ID NO: 100 |
| SEQ ID NO: 28 | SEQ ID NO: 99 |
| SEQ ID NO: 28 | SEQ ID NO: 101 |
| SEQ ID NO: 28 | SEQ ID NO: 102 |
| SEQ ID NO: 29 | SEQ ID NO: 103 |
| SEQ ID NO: 29 | SEQ ID NO: 104 |
| SEQ ID NO: 29 | SEQ ID NO: 105 |
| SEQ ID NO: 30 | SEQ ID NO: 106 |
| SEQ ID NO: 30 | SEQ ID NO: 107 |
| SEQ ID NO: 30 | SEQ ID NO: 108 |
| SEQ ID NO: 31 | SEQ ID NO: 109 |
| SEQ ID NO: 31 | SEQ ID NO: 110 |
| SEQ ID NO: 31 | SEQ ID NO: 111 |
| SEQ ID NO: 32 | SEQ ID NO: 112 |
| SEQ ID NO: 33 | SEQ ID NO: 112 |
| SEQ ID NO: 34 | SEQ ID NO: 113 |
| SEQ ID NO: 34 | SEQ ID NO: 114 |
| SEQ ID NO: 34 | SEQ ID NO: 115 |
| SEQ ID NO: 35 | SEQ ID NO: 116 |
| SEQ ID NO: 35 | SEQ ID NO: 117 |
| SEQ ID NO: 35 | SEQ ID NO: 118 |
| SEQ ID NO: 36 | SEQ ID NO: 119 |
| SEQ ID NO: 36 | SEQ ID NO: 120 |
| SEQ ID NO: 36 | SEQ ID NO: 121 |
| SEQ ID NO: 37 | SEQ ID NO: 122 |
| SEQ ID NO: 37 | SEQ ID NO: 123 |
| SEQ ID NO: 38 | SEQ ID NO: 122 |
| SEQ ID NO: 38 | SEQ ID NO: 123 |

In some embodiments, the nucleic acid sequences of the mgRNA and the hgRNA comprise respectively:

TABLE 29

| mgRNA | hgRNA |
|---|---|
| SEQ ID NO: 691 | SEQ ID NO: 692 |
| SEQ ID NO: 693 | SEQ ID NO: 694; or |
| SEQ ID NO: 695 | SEQ ID NO: 696 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a cytotoxic T-lymphocyte associated protein 4 (CTLA4) gene and the hgRNA comprises a hgRNA spacer, wherein the nucleic acid sequence of the mgRNA spacer comprises a sequence selected from SEQ ID NOs: 247-256.

In some embodiments, the nucleic acid sequences of the mgRNA spacer and the hgRNA spacer comprise the sequences as set forth in Table 13.

TABLE 13

| mgRNA | hgRNA |
|---|---|
| SEQ ID NO: 247 | SEQ ID NO: 257 |
| SEQ ID NO: 247 | SEQ ID NO: 258 |
| SEQ ID NO: 248 | SEQ ID NO: 259 |
| SEQ ID NO: 249 | SEQ ID NO: 260 |
| SEQ ID NO: 249 | SEQ ID NO: 261 |
| SEQ ID NO: 250 | SEQ ID NO: 262 |
| SEQ ID NO: 250 | SEQ ID NO: 263 |
| SEQ ID NO: 250 | SEQ ID NO: 264 |
| SEQ ID NO: 251 | SEQ ID NO: 265 |
| SEQ ID NO: 251 | SEQ ID NO: 266 |
| SEQ ID NO: 251 | SEQ ID NO: 267 |
| SEQ ID NO: 252 | SEQ ID NO: 268 |
| SEQ ID NO: 252 | SEQ ID NO: 269 |
| SEQ ID NO: 253 | SEQ ID NO: 268 |
| SEQ ID NO: 253 | SEQ ID NO: 269 |
| SEQ ID NO: 253 | SEQ ID NO: 270 |
| SEQ ID NO: 254 | SEQ ID NO: 271 |
| SEQ ID NO: 254 | SEQ ID NO: 272 |
| SEQ ID NO: 255 | SEQ ID NO: 273 |
| SEQ ID NO: 255 | SEQ ID NO: 274 |
| SEQ ID NO: 255 | SEQ ID NO: 275 |
| SEQ ID NO: 256 | SEQ ID NO: 276 |
| SEQ ID NO: 256 | SEQ ID NO: 277 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a T cell immunoreceptor with Ig and ITIM domains (TIGIT) gene and the hgRNA comprises a hgRNA spacer, wherein the nucleic acid sequence of the mgRNA spacer comprises a sequence selected from SEQ ID NOs: 278-294.

In some embodiments, the nucleic acid sequences of the mgRNA spacer and the hgRNA spacer comprise the sequences as set forth in Table 14.

TABLE 14

| mgRNA | hgRNA |
|---|---|
| SEQ ID NO: 278 | SEQ ID NO: 295 |
| SEQ ID NO: 278 | SEQ ID NO: 296 |
| SEQ ID NO: 278 | SEQ ID NO: 297 |
| SEQ ID NO: 279 | SEQ ID NO: 295 |
| SEQ ID NO: 279 | SEQ ID NO: 296 |
| SEQ ID NO: 279 | SEQ ID NO: 297 |
| SEQ ID NO: 280 | SEQ ID NO: 298 |
| SEQ ID NO: 280 | SEQ ID NO: 299 |
| SEQ ID NO: 280 | SEQ ID NO: 300 |
| SEQ ID NO: 281 | SEQ ID NO: 298 |
| SEQ ID NO: 281 | SEQ ID NO: 299 |
| SEQ ID NO: 281 | SEQ ID NO: 300 |
| SEQ ID NO: 282 | SEQ ID NO: 301 |
| SEQ ID NO: 282 | SEQ ID NO: 302 |
| SEQ ID NO: 282 | SEQ ID NO: 303 |
| SEQ ID NO: 283 | SEQ ID NO: 301 |
| SEQ ID NO: 283 | SEQ ID NO: 302 |
| SEQ ID NO: 283 | SEQ ID NO: 303 |
| SEQ ID NO: 284 | SEQ ID NO: 304 |
| SEQ ID NO: 284 | SEQ ID NO: 305 |
| SEQ ID NO: 285 | SEQ ID NO: 307 |
| SEQ ID NO: 285 | SEQ ID NO: 308 |
| SEQ ID NO: 286 | SEQ ID NO: 306 |
| SEQ ID NO: 286 | SEQ ID NO: 307 |
| SEQ ID NO: 286 | SEQ ID NO: 308 |
| SEQ ID NO: 287 | SEQ ID NO: 306 |
| SEQ ID NO: 287 | SEQ ID NO: 307 |
| SEQ ID NO: 287 | SEQ ID NO: 308 |
| SEQ ID NO: 288 | SEQ ID NO: 309 |
| SEQ ID NO: 288 | SEQ ID NO: 310 |
| SEQ ID NO: 289 | SEQ ID NO: 309 |
| SEQ ID NO: 289 | SEQ ID NO: 310 |
| SEQ ID NO: 289 | SEQ ID NO: 311 |
| SEQ ID NO: 290 | SEQ ID NO: 312 |
| SEQ ID NO: 290 | SEQ ID NO: 313 |
| SEQ ID NO: 290 | SEQ ID NO: 314 |
| SEQ ID NO: 291 | SEQ ID NO: 315 |
| SEQ ID NO: 291 | SEQ ID NO: 316 |
| SEQ ID NO: 291 | SEQ ID NO: 317 |
| SEQ ID NO: 292 | SEQ ID NO: 318 |
| SEQ ID NO: 292 | SEQ ID NO: 319 |
| SEQ ID NO: 292 | SEQ ID NO: 320 |
| SEQ ID NO: 293 | SEQ ID NO: 318 |
| SEQ ID NO: 293 | SEQ ID NO: 319 |
| SEQ ID NO: 293 | SEQ ID NO: 320 |
| SEQ ID NO: 294 | SEQ ID NO: 321 |
| SEQ ID NO: 294 | SEQ ID NO: 322 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a hepatitis A virus cellular receptor 2 (HAVCR2/TIM3) gene and the hgRNA comprises a hgRNA spacer, wherein the nucleic acid sequence of the mgRNA spacer comprises a sequence selected from SEQ ID NOs: 323-337.

In some embodiments, the nucleic acid sequences of the mgRNA spacer and the hgRNA spacer comprise the sequences as set forth in Table 15.

TABLE 15

| mgRNA | hgRNA |
|---|---|
| SEQ ID NO: 323 | SEQ ID NO: 338 |
| SEQ ID NO: 323 | SEQ ID NO: 339 |
| SEQ ID NO: 324 | SEQ ID NO: 340 |
| SEQ ID NO: 324 | SEQ ID NO: 341 |
| SEQ ID NO: 325 | SEQ ID NO: 340 |
| SEQ ID NO: 325 | SEQ ID NO: 341 |
| SEQ ID NO: 325 | SEQ ID NO: 342 |
| SEQ ID NO: 326 | SEQ ID NO: 343 |
| SEQ ID NO: 326 | SEQ ID NO: 344 |
| SEQ ID NO: 326 | SEQ ID NO: 345 |
| SEQ ID NO: 327 | SEQ ID NO: 346 |
| SEQ ID NO: 327 | SEQ ID NO: 347 |
| SEQ ID NO: 328 | SEQ ID NO: 348 |
| SEQ ID NO: 328 | SEQ ID NO: 349 |
| SEQ ID NO: 328 | SEQ ID NO: 350 |
| SEQ ID NO: 329 | SEQ ID NO: 351 |
| SEQ ID NO: 329 | SEQ ID NO: 352 |
| SEQ ID NO: 329 | SEQ ID NO: 353 |
| SEQ ID NO: 329 | SEQ ID NO: 354 |
| SEQ ID NO: 330 | SEQ ID NO: 351 |
| SEQ ID NO: 330 | SEQ ID NO: 352 |
| SEQ ID NO: 330 | SEQ ID NO: 353 |
| SEQ ID NO: 330 | SEQ ID NO: 354 |
| SEQ ID NO: 331 | SEQ ID NO: 355 |
| SEQ ID NO: 331 | SEQ ID NO: 356 |
| SEQ ID NO: 332 | SEQ ID NO: 357 |
| SEQ ID NO: 332 | SEQ ID NO: 358 |
| SEQ ID NO: 332 | SEQ ID NO: 359 |
| SEQ ID NO: 333 | SEQ ID NO: 357 |
| SEQ ID NO: 333 | SEQ ID NO: 358 |
| SEQ ID NO: 333 | SEQ ID NO: 359 |
| SEQ ID NO: 334 | SEQ ID NO: 357 |
| SEQ ID NO: 334 | SEQ ID NO: 358 |
| SEQ ID NO: 334 | SEQ ID NO: 359 |
| SEQ ID NO: 335 | SEQ ID NO: 360 |
| SEQ ID NO: 336 | SEQ ID NO: 361 |
| SEQ ID NO: 336 | SEQ ID NO: 362 |
| SEQ ID NO: 336 | SEQ ID NO: 363 |
| SEQ ID NO: 337 | SEQ ID NO: 361 |
| SEQ ID NO: 337 | SEQ ID NO: 362 |
| SEQ ID NO: 337 | SEQ ID NO: 363 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a lymphocyte activating 3 (LAG3) gene and the hgRNA comprises a hgRNA spacer, wherein the nucleic acid sequence of the mgRNA spacer comprises a sequence selected from SEQ ID NOs: 364-396.

In some embodiments, the nucleic acid sequences of the mgRNA spacer and the hgRNA spacer comprise the sequences as set forth in Table 16.

TABLE 16

| mgRNA | hgRNA |
|---|---|
| SEQ ID NO: 364 | SEQ ID NO: 397 |
| SEQ ID NO: 364 | SEQ ID NO: 398 |
| SEQ ID NO: 365 | SEQ ID NO: 399 |
| SEQ ID NO: 365 | SEQ ID NO: 400 |
| SEQ ID NO: 365 | SEQ ID NO: 401 |
| SEQ ID NO: 366 | SEQ ID NO: 402 |
| SEQ ID NO: 366 | SEQ ID NO: 403 |
| SEQ ID NO: 366 | SEQ ID NO: 404 |
| SEQ ID NO: 367 | SEQ ID NO: 405 |
| SEQ ID NO: 367 | SEQ ID NO: 406 |
| SEQ ID NO: 367 | SEQ ID NO: 407 |
| SEQ ID NO: 368 | SEQ ID NO: 408 |
| SEQ ID NO: 368 | SEQ ID NO: 409 |
| SEQ ID NO: 369 | SEQ ID NO: 410 |
| SEQ ID NO: 370 | SEQ ID NO: 411 |
| SEQ ID NO: 371 | SEQ ID NO: 412 |
| SEQ ID NO: 371 | SEQ ID NO: 413 |
| SEQ ID NO: 371 | SEQ ID NO: 414 |
| SEQ ID NO: 372 | SEQ ID NO: 415 |
| SEQ ID NO: 372 | SEQ ID NO: 416 |
| SEQ ID NO: 373 | SEQ ID NO: 417 |
| SEQ ID NO: 373 | SEQ ID NO: 418 |
| SEQ ID NO: 373 | SEQ ID NO: 419 |
| SEQ ID NO: 374 | SEQ ID NO: 417 |
| SEQ ID NO: 374 | SEQ ID NO: 418 |
| SEQ ID NO: 374 | SEQ ID NO: 420 |
| SEQ ID NO: 375 | SEQ ID NO: 421 |
| SEQ ID NO: 375 | SEQ ID NO: 422 |
| SEQ ID NO: 376 | SEQ ID NO: 423 |
| SEQ ID NO: 376 | SEQ ID NO: 424 |
| SEQ ID NO: 376 | SEQ ID NO: 425 |
| SEQ ID NO: 377 | SEQ ID NO: 426 |
| SEQ ID NO: 377 | SEQ ID NO: 427 |
| SEQ ID NO: 377 | SEQ ID NO: 428 |
| SEQ ID NO: 378 | SEQ ID NO: 429 |
| SEQ ID NO: 379 | SEQ ID NO: 430 |
| SEQ ID NO: 379 | SEQ ID NO: 431 |
| SEQ ID NO: 380 | SEQ ID NO: 432 |
| SEQ ID NO: 380 | SEQ ID NO: 433 |
| SEQ ID NO: 381 | SEQ ID NO: 434 |
| SEQ ID NO: 381 | SEQ ID NO: 435 |
| SEQ ID NO: 381 | SEQ ID NO: 436 |
| SEQ ID NO: 382 | SEQ ID NO: 437 |
| SEQ ID NO: 382 | SEQ ID NO: 438 |
| SEQ ID NO: 382 | SEQ ID NO: 439 |
| SEQ ID NO: 383 | SEQ ID NO: 440 |
| SEQ ID NO: 383 | SEQ ID NO: 441 |
| SEQ ID NO: 383 | SEQ ID NO: 442 |
| SEQ ID NO: 384 | SEQ ID NO: 443 |
| SEQ ID NO: 384 | SEQ ID NO: 444 |
| SEQ ID NO: 384 | SEQ ID NO: 445 |
| SEQ ID NO: 385 | SEQ ID NO: 446 |
| SEQ ID NO: 385 | SEQ ID NO: 447 |
| SEQ ID NO: 386 | SEQ ID NO: 448 |
| SEQ ID NO: 386 | SEQ ID NO: 449 |
| SEQ ID NO: 386 | SEQ ID NO: 450 |
| SEQ ID NO: 387 | SEQ ID NO: 451 |
| SEQ ID NO: 387 | SEQ ID NO: 452 |
| SEQ ID NO: 387 | SEQ ID NO: 453 |
| SEQ ID NO: 388 | SEQ ID NO: 451 |
| SEQ ID NO: 388 | SEQ ID NO: 454 |
| SEQ ID NO: 388 | SEQ ID NO: 455 |
| SEQ ID NO: 389 | SEQ ID NO: 454 |
| SEQ ID NO: 389 | SEQ ID NO: 455 |
| SEQ ID NO: 389 | SEQ ID NO: 456 |
| SEQ ID NO: 390 | SEQ ID NO: 454 |
| SEQ ID NO: 390 | SEQ ID NO: 455 |
| SEQ ID NO: 390 | SEQ ID NO: 456 |
| SEQ ID NO: 391 | SEQ ID NO: 457 |
| SEQ ID NO: 391 | SEQ ID NO: 458 |
| SEQ ID NO: 391 | SEQ ID NO: 459 |
| SEQ ID NO: 392 | SEQ ID NO: 460 |
| SEQ ID NO: 392 | SEQ ID NO: 461 |
| SEQ ID NO: 392 | SEQ ID NO: 462 |
| SEQ ID NO: 393 | SEQ ID NO: 463 |
| SEQ ID NO: 393 | SEQ ID NO: 464 |
| SEQ ID NO: 393 | SEQ ID NO: 465 |
| SEQ ID NO: 394 | SEQ ID NO: 466 |
| SEQ ID NO: 394 | SEQ ID NO: 467 |
| SEQ ID NO: 394 | SEQ ID NO: 468 |
| SEQ ID NO: 395 | SEQ ID NO: 469 |
| SEQ ID NO: 395 | SEQ ID NO: 470 |
| SEQ ID NO: 396 | SEQ ID NO: 469 |
| SEQ ID NO: 396 | SEQ ID NO: 470 |
| SEQ ID NO: 396 | SEQ ID NO: 471 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a cytokine inducible SH2 containing protein (CISH) gene and the hgRNA comprises a hgRNA spacer, wherein the nucleic acid sequence of the mgRNA spacer comprises a sequence selected from SEQ ID NOs: 472-482.

In some embodiments, the nucleic acid sequences of the mgRNA spacer and the hgRNA spacer comprise the sequences as set forth in Table 17.

TABLE 17

| mgRNA | hgRNA |
|---|---|
| SEQ ID NO: 472 | SEQ ID NO: 483 |
| SEQ ID NO: 472 | SEQ ID NO: 484 |
| SEQ ID NO: 472 | SEQ ID NO: 485 |
| SEQ ID NO: 473 | SEQ ID NO: 486 |
| SEQ ID NO: 473 | SEQ ID NO: 487 |
| SEQ ID NO: 473 | SEQ ID NO: 488 |
| SEQ ID NO: 474 | SEQ ID NO: 486 |
| SEQ ID NO: 474 | SEQ ID NO: 487 |
| SEQ ID NO: 474 | SEQ ID NO: 488 |
| SEQ ID NO: 475 | SEQ ID NO: 489 |
| SEQ ID NO: 475 | SEQ ID NO: 490 |
| SEQ ID NO: 475 | SEQ ID NO: 491 |
| SEQ ID NO: 476 | SEQ ID NO: 492 |
| SEQ ID NO: 476 | SEQ ID NO: 493 |
| SEQ ID NO: 477 | SEQ ID NO: 492 |
| SEQ ID NO: 477 | SEQ ID NO: 493 |
| SEQ ID NO: 478 | SEQ ID NO: 494 |
| SEQ ID NO: 478 | SEQ ID NO: 495 |
| SEQ ID NO: 478 | SEQ ID NO: 496 |
| SEQ ID NO: 479 | SEQ ID NO: 494 |
| SEQ ID NO: 479 | SEQ ID NO: 495 |
| SEQ ID NO: 479 | SEQ ID NO: 496 |
| SEQ ID NO: 480 | SEQ ID NO: 494 |
| SEQ ID NO: 480 | SEQ ID NO: 495 |
| SEQ ID NO: 480 | SEQ ID NO: 497 |
| SEQ ID NO: 481 | SEQ ID NO: 498 |
| SEQ ID NO: 481 | SEQ ID NO: 499 |
| SEQ ID NO: 481 | SEQ ID NO: 500 |
| SEQ ID NO: 482 | SEQ ID NO: 501 |
| SEQ ID NO: 482 | SEQ ID NO: 502 |
| SEQ ID NO: 482 | SEQ ID NO: 503 |

In some embodiments, the nucleic acid sequences of the mgRNA and the hgRNA comprise respectively:

TABLE 30

| mgRNA | hgRNA |
|---|---|
| SEQ ID NO: 701 | SEQ ID NO: 702 |
| SEQ ID NO: 701 | SEQ ID NO: 703 |
| SEQ ID NO: 704 | SEQ ID NO: 705 |
| SEQ ID NO: 704 | SEQ ID NO: 706; or |
| SEQ ID NO: 707 | SEQ ID NO: 708 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a transforming growth factor beta receptor 2 (TGFBR2) gene and the hgRNA comprises a hgRNA spacer, wherein the nucleic acid sequence of the mgRNA spacer comprises a sequence selected from SEQ ID NOs: 504-510.

In some embodiments, the nucleic acid sequences of the mgRNA spacer and the hgRNA spacer comprise the sequences as set forth in Table 18.

TABLE 18

| mgRNA | hgRNA |
|---|---|
| SEQ ID NO: 504 | SEQ ID NO: 511 |
| SEQ ID NO: 504 | SEQ ID NO: 512 |
| SEQ ID NO: 505 | SEQ ID NO: 513 |
| SEQ ID NO: 505 | SEQ ID NO: 514 |
| SEQ ID NO: 505 | SEQ ID NO: 515 |
| SEQ ID NO: 505 | SEQ ID NO: 516 |
| SEQ ID NO: 506 | SEQ ID NO: 517 |
| SEQ ID NO: 506 | SEQ ID NO: 518 |
| SEQ ID NO: 507 | SEQ ID NO: 519 |
| SEQ ID NO: 507 | SEQ ID NO: 520 |
| SEQ ID NO: 507 | SEQ ID NO: 521 |
| SEQ ID NO: 507 | SEQ ID NO: 522 |
| SEQ ID NO: 508 | SEQ ID NO: 523 |
| SEQ ID NO: 508 | SEQ ID NO: 524 |
| SEQ ID NO: 508 | SEQ ID NO: 525 |
| SEQ ID NO: 508 | SEQ ID NO: 526 |
| SEQ ID NO: 509 | SEQ ID NO: 527 |
| SEQ ID NO: 510 | SEQ ID NO: 528 |
| SEQ ID NO: 510 | SEQ ID NO: 529 |

In some embodiments, the nucleic acid sequences of the mgRNA and the hgRNA comprise respectively:

TABLE 31

| mgRNA | hgRNA |
|---|---|
| SEQ ID NO: 709 | SEQ ID NO: 710 |
| SEQ ID NO: 709 | SEQ ID NO: 711 |
| SEQ ID NO: 712 | SEQ ID NO: 713; or |
| SEQ ID NO: 712 | SEQ ID NO: 714 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a Fas cell surface death receptor (FAS) gene and the hgRNA comprises a hgRNA spacer, wherein the nucleic acid sequence of the mgRNA spacer comprises a sequence selected from SEQ ID NOs: 530-541.

In some embodiments, the nucleic acid sequences of the mgRNA spacer and the hgRNA spacer comprise the sequences as set forth in Table 19.

TABLE 19

| mgRNA | hgRNA |
|---|---|
| SEQ ID NO: 530 | SEQ ID NO: 542 |
| SEQ ID NO: 530 | SEQ ID NO: 543 |
| SEQ ID NO: 530 | SEQ ID NO: 544 |
| SEQ ID NO: 531 | SEQ ID NO: 545 |
| SEQ ID NO: 531 | SEQ ID NO: 546 |
| SEQ ID NO: 532 | SEQ ID NO: 547 |
| SEQ ID NO: 532 | SEQ ID NO: 548 |
| SEQ ID NO: 532 | SEQ ID NO: 549 |
| SEQ ID NO: 533 | SEQ ID NO: 550 |
| SEQ ID NO: 534 | SEQ ID NO: 551 |
| SEQ ID NO: 534 | SEQ ID NO: 552 |
| SEQ ID NO: 534 | SEQ ID NO: 553 |
| SEQ ID NO: 535 | SEQ ID NO: 554 |
| SEQ ID NO: 535 | SEQ ID NO: 555 |
| SEQ ID NO: 535 | SEQ ID NO: 556 |
| SEQ ID NO: 536 | SEQ ID NO: 557 |
| SEQ ID NO: 536 | SEQ ID NO: 558 |
| SEQ ID NO: 537 | SEQ ID NO: 559 |
| SEQ ID NO: 537 | SEQ ID NO: 560 |
| SEQ ID NO: 537 | SEQ ID NO: 561 |
| SEQ ID NO: 538 | SEQ ID NO: 562 |
| SEQ ID NO: 538 | SEQ ID NO: 563 |
| SEQ ID NO: 539 | SEQ ID NO: 562 |
| SEQ ID NO: 539 | SEQ ID NO: 563 |
| SEQ ID NO: 540 | SEQ ID NO: 564 |
| SEQ ID NO: 541 | SEQ ID NO: 564 |

In some embodiments, the nucleic acid sequences of the mgRNA and the hgRNA comprise respectively:

TABLE 32

| mgRNA | hgRNA |
|---|---|
| SEQ ID NO: 715 | SEQ ID NO: 716; or |
| SEQ ID NO: 717 | SEQ ID NO: 718 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a CD7 gene and the hgRNA comprises a hgRNA spacer, wherein the nucleic acid sequence of the mgRNA spacer comprises a sequence selected from SEQ ID NOs: 565-575.

In some embodiments, the nucleic acid sequences of the mgRNA spacer and the hgRNA spacer comprise the sequences as set forth in Table 20.

TABLE 20

| mgRNA | hgRNA |
|---|---|
| SEQ ID NO: 565 | SEQ ID NO: 576 |
| SEQ ID NO: 565 | SEQ ID NO: 577 |
| SEQ ID NO: 565 | SEQ ID NO: 578 |
| SEQ ID NO: 565 | SEQ ID NO: 579 |
| SEQ ID NO: 566 | SEQ ID NO: 580 |
| SEQ ID NO: 566 | SEQ ID NO: 581 |
| SEQ ID NO: 566 | SEQ ID NO: 582 |
| SEQ ID NO: 566 | SEQ ID NO: 583 |
| SEQ ID NO: 567 | SEQ ID NO: 584 |
| SEQ ID NO: 567 | SEQ ID NO: 585 |
| SEQ ID NO: 567 | SEQ ID NO: 586 |
| SEQ ID NO: 567 | SEQ ID NO: 587 |
| SEQ ID NO: 568 | SEQ ID NO: 588 |
| SEQ ID NO: 568 | SEQ ID NO: 589 |
| SEQ ID NO: 568 | SEQ ID NO: 590 |
| SEQ ID NO: 569 | SEQ ID NO: 591 |
| SEQ ID NO: 569 | SEQ ID NO: 592 |
| SEQ ID NO: 569 | SEQ ID NO: 593 |
| SEQ ID NO: 570 | SEQ ID NO: 591 |
| SEQ ID NO: 570 | SEQ ID NO: 594 |
| SEQ ID NO: 571 | SEQ ID NO: 595 |
| SEQ ID NO: 571 | SEQ ID NO: 596 |
| SEQ ID NO: 571 | SEQ ID NO: 597 |
| SEQ ID NO: 571 | SEQ ID NO: 598 |
| SEQ ID NO: 572 | SEQ ID NO: 599 |
| SEQ ID NO: 572 | SEQ ID NO: 600 |
| SEQ ID NO: 573 | SEQ ID NO: 599 |
| SEQ ID NO: 573 | SEQ ID NO: 600 |
| SEQ ID NO: 573 | SEQ ID NO: 601 |
| SEQ ID NO: 574 | SEQ ID NO: 602 |
| SEQ ID NO: 574 | SEQ ID NO: 603 |
| SEQ ID NO: 574 | SEQ ID NO: 604 |
| SEQ ID NO: 574 | SEQ ID NO: 605 |
| SEQ ID NO: 575 | SEQ ID NO: 606 |
| SEQ ID NO: 575 | SEQ ID NO: 607 |
| SEQ ID NO: 575 | SEQ ID NO: 608 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a CBLB gene and the hgRNA comprises a hgRNA spacer, wherein the nucleic acid sequence of the mgRNA spacer comprises a sequence selected from SEQ ID NOs: 609-618.

In some embodiments, the nucleic acid sequences of the mgRNA spacer and the h RA spacer comprise the sequences as set forth in Table 21.

TABLE 21

| mgRNA | hgRNA |
|---|---|
| SEQ ID NO. 609 | SEQ ID NO. 619 |
| SEQ ID NO. 609 | SEQ ID NO. 620 |
| SEQ ID NO. 610 | SEQ ID NO. 621 |
| SEQ ID NO. 610 | SEQ ID NO. 622 |
| SEQ ID NO. 610 | SEQ ID NO. 623 |
| SEQ ID NO. 611 | SEQ ID NO. 624 |
| SEQ ID NO. 612 | SEQ ID NO. 625 |
| SEQ ID NO. 612 | SEQ ID NO. 626 |
| SEQ ID NO. 613 | SEQ ID NO. 627 |
| SEQ ID NO. 614 | SEQ ID NO. 628 |
| SEQ ID NO. 615 | SEQ ID NO. 629 |
| SEQ ID NO. 615 | SEQ ID NO. 630 |
| SEQ ID NO. 615 | SEQ ID NO. 631 |
| SEQ ID NO. 616 | SEQ ID NO. 632 |
| SEQ ID NO. 616 | SEQ ID NO. 633 |
| SEQ ID NO. 617 | SEQ ID NO. 634 |
| SEQ ID NO. 617 | SEQ ID NO. 635 |
| SEQ ID NO. 618 | SEQ ID NO. 636 |

In some embodiments, the nucleic acid sequences of the mgRNA and the hgRNA comprise respectively:

TABLE 33

| mgRNA | hgRNA |
|---|---|
| SEQ ID NO: 723 | SEQ ID NO: 724; or |
| SEQ ID NO: 725 | SEQ ID NO: 726 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a KLRC1 gene and the hgRNA comprises a hgRNA spacer, wherein the nucleic acid sequence of the mgRNA spacer comprises a sequence selected from SEQ ID NOs: 637-641.

In some embodiments, the nucleic acid sequences of the mgRNA spacer and the hgRNA spacer comprise the sequences as set forth in Table 22.

TABLE 22

| mgRNA | hgRNA |
|---|---|
| SEQ ID NO. 637 | SEQ ID NO. 642 |
| SEQ ID NO. 637 | SEQ ID NO. 643 |
| SEQ ID NO. 638 | SEQ ID NO. 644 |
| SEQ ID NO. 638 | SEQ ID NO. 645 |
| SEQ ID NO. 639 | SEQ ID NO. 646 |
| SEQ ID NO. 639 | SEQ ID NO. 647 |
| SEQ ID NO. 640 | SEQ ID NO. 648 |
| SEQ ID NO. 640 | SEQ ID NO. 649 |
| SEQ ID NO. 641 | SEQ ID NO. 650 |

In some embodiments, the nucleic acid sequences of the mgRNA and the hgRNA comprise respectively:

TABLE 34

| mgRNA | hgRNA |
|---|---|
| SEQ ID NO: 727 | SEQ ID NO: 728; or |
| SEQ ID NO: 729 | SEQ ID NO: 730 |

In an aspect, the present disclosure provides a gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a CD38 gene and the hgRNA comprises a hgRNA spacer, wherein the nucleic acid sequence of the mgRNA spacer comprises a sequence selected from SEQ ID NOs: 651-659.

In some embodiments, the nucleic acid sequences of the mgRNA spacer and the hgRNA spacer comprise the sequences as set forth in Table 23.

TABLE 23

| mgRNA | hgRNA |
|---|---|
| SEQ ID NO. 651 | SEQ ID NO. 660 |
| SEQ ID NO. 651 | SEQ ID NO. 661 |
| SEQ ID NO. 651 | SEQ ID NO. 662 |
| SEQ ID NO. 652 | SEQ ID NO. 663 |
| SEQ ID NO. 652 | SEQ ID NO. 664 |
| SEQ ID NO. 652 | SEQ ID NO. 665 |
| SEQ ID NO. 653 | SEQ ID NO. 666 |
| SEQ ID NO. 654 | SEQ ID NO. 667 |
| SEQ ID NO. 655 | SEQ ID NO. 668 |
| SEQ ID NO. 655 | SEQ ID NO. 669 |
| SEQ ID NO. 655 | SEQ ID NO. 670 |
| SEQ ID NO. 656 | SEQ ID NO. 671 |
| SEQ ID NO. 657 | SEQ ID NO. 672 |
| SEQ ID NO. 657 | SEQ ID NO. 673 |
| SEQ ID NO. 658 | SEQ ID NO. 674 |
| SEQ ID NO. 658 | SEQ ID NO. 675 |
| SEQ ID NO. 659 | SEQ ID NO. 676 |
| SEQ ID NO. 659 | SEQ ID NO. 677 |
| SEQ ID NO. 659 | SEQ ID NO. 678 |

In some embodiments, the nucleic acid sequences of the mgRNA and the hgRNA comprise respectively:

TABLE 35

| mgRNA | hgRNA |
|---|---|
| SEQ ID NO: 719 | SEQ ID NO: 720; or |
| SEQ ID NO: 721 | SEQ ID NO: 722 |

In some embodiments, the gene editing system described herein comprises a first mgRNA comprising a first mgRNA spacer targeting a first gene, and a second mgRNA comprising a second mgRNA spacer targeting a second gene, wherein the first gene and the second gene are each selected from the group consisting of the TRAC gene, the B2M gene, the CD52 gene, the PDCD1 gene, CTLA4 gene, TTGIT gene, TIM3 gene, LAG3 gene, CISH gene, TGFBR2 gene, FAS gene, CD7 gene, CBLB gene, KLRC1 gene, and CD38 gene. In some embodiments, the first gene and the second gene are different.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, and (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, and wherein the first Cas protein and second Cas protein are the same or different.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, and (6) a protease, or a polynucleotide encoding the protease, and (7) a nucleobase deaminase inhibitor domain, wherein the first Cas protein and second Cas protein are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, (6) a protease, or a polynucleotide encoding the protease, (7) a nucleobase deaminase inhibitor domain, and (8) a second fusion protein comprising the protease and a second RNA binding domain, or a polynucleotide encoding the second fusion protein, wherein the first Cas protein and second Cas protein are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof, wherein the protease and the second RNA binding domain are optionally connected by a linker, wherein the mgRNA further comprises a second protein-binding motif, and wherein the second RNA binding domain binds to the second protein-binding motif.

In some embodiments, the protease is split into a first protease fragment and a second protease fragment, wherein the first or second protease fragment alone is not able to cleave the cleavage site.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, (6) a protease, or a polynucleotide encoding the protease, (7) a nucleobase deaminase inhibitor domain, (8) a second fusion protein comprising the first protease fragment and a second RNA binding domain, or a polynucleotide encoding the second fusion protein, wherein the first protease fragment and the second RNA binding domain are optionally connected by a linker, and (9) a third fusion protein comprising the second protease fragment and a third RNA binding domain, or a polynucleotide encoding the third fusion protein, wherein the second as protease fragment and the third RNA binding domain are optionally connected by a linker, wherein the first Cas protein and second Cas protein are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof, wherein the mgRNA further comprises a second protein-binding motif and a third protein-binding motif, wherein the second RNA binding domain binds to the second protein-binding motif, and wherein the third RNA binding domain binds to the third protein-binding motif.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the first Cas protein binds to the first CRISPR motif, (4) a second Cas protein, or a polynucleotide encoding the second Cas protein, wherein the second Cas protein binds to the second CRISPR motif, (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, (6) a protease, or a polynucleotide encoding the protease, (7) a nucleobase deaminase inhibitor domain, (8) a second fusion protein comprising the first protease fragment and a second RNA binding domain, or a polynucleotide encoding the second fusion protein, wherein the first protease fragment and the second RNA binding domain are optionally connected by a linker, and (9) a third fusion protein comprising the second protease fragment and a third RNA binding domain, or a polynucleotide encoding the third fusion protein, wherein the second protease fragment and the third RNA binding domain are optionally connected by a linker, wherein the first Cas protein and second Cas protein are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof, wherein the mgRNA further comprises a second protein-binding motif and a third protein-binding motif, wherein the second RNA binding domain binds to the second protein-binding motif, wherein the third RNA binding domain binds to the third protein-binding motif, and wherein the second and third RNA binding domains are the same or different, and the second and third protein-binding motifs are the same or different.

In some embodiments, the gene editing system disclosed herein comprises (1) the hgRNA comprising a first CRISPR motif, the hgRNA spacer, and a first protein-binding motif, or a DNA polynucleotide encoding the hgRNA, (2) the mgRNA comprising a second CRISPR motif and the mgRNA spacer, or a DNA polynucleotide encoding the mgRNA, (3) a first CRISPR-associated protein (Cas protein), or a polynucleotide encoding the first Cas protein, wherein the as first Cas protein binds to the first CRISPR motif, (4) a second Cas protein, or a polynucleotide encoding the second Cas protein, wherein the second Cas protein binds to the second CRISPR motif, (5) a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif, (6) a protease, or a polynucleotide encoding the protease, (7) a nucleobase deaminase inhibitor domain, and (8) a second fusion protein comprising the first protease fragment and a second RNA binding domain, or a polynucleotide encoding the second fusion protein, wherein the first Cas protein and second Cas protein are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof, wherein the first protease fragment and the second RNA binding domain are optionally connected by a linker, wherein the mgRNA further comprises a second protein-binding motif, and wherein the second RNA binding domain binds to the second protein-binding motif.

A “protease” refers to an enzyme that catalyzes proteolysis. A “cleavage site for a protease” refers to a short peptide that the protease recognizes, and within the short peptide creates a proteolytic cleavage. Non-limiting examples of proteases include TEV protease, TuMV protease, PPV protease, PVY protease, ZIKV protease, and WNV protease. The protein sequences of example proteases and their corresponding cleavage sites are provided in Table 6.

TABLE 6
Exemplary proteases and their cleavage sites

| Name | Sequence | SEQ ID NO |
|---|---|---|
| TEV protease | MGESLFKGPRDYNPISSTICHLTNESDGHTTSLYGIGFGPFIITNKHLFRR | SEQ ID NO: 124 |
| | NNGTLLVQSLHGVFKVKNTTTLQQHLIDGRDMIIIRMPKDFPPFPQKLK | |
| | FREPQREERICLVTTNFQTKSMSSMVSDTSCTFPSSDGIFWKHWIQTKD | |
| | GQCGSPLVSTRDGFIVGIHSASNFTNTNNYFTSVPKNFMELLTNQEAQ | |
| | QWVMGWRLNADSVLWGGHKVFMVKPEEPFQPVKEATQLMN | |
| TEV protease N- | MGESLFKGPRDYNPISSTICHLTNESDGHTTSLYGIGFGPFIITNKHLFRR | SEQ ID NO: 125 |
| terminal domain | NNGTLLVQSLHGVFKVKNTTTLQQHLIDGRDMIIIRMPKDFPPFPQKLK | |
| | FREPQREERICLVTTNFQT | |
| TEV protease C- | MKSMSSMVSDTSCTFPSSDGIFWKHWIQTKDGQCGSPLVSTRDGFIV | SEQ ID NO: 126 |
| terminal domain | GIHSASNFTNTNNYFTSVPKNFMELLTNQEAQQWVMGWRLNADSVL | |
| | WGGHKVFMVKPEEPFQPVKEATQ | |
| TEV protease | ENLYFQS | SEQ ID NO: 127 |
| cleavage site | | |
| TuMV protease | MASSNSMFRGLRDYNPISNNICHLTNVSDGASNSLYGVGFGPLILTNRH | SEQ ID NO: 128 |
| | LFERNNGELVIKSRHGEFVIKNTTQLHLLPIPDRDLLLIRLPKDVPPFPQKL | |
| | GFRQPEKGERICMVGSNFQTKSITSIVSETSTIMPVENSQFWKHWISTK | |
| | DGQCGSPMVSTKDGKILGLHSLANFQNSINYFAAFPDDFAEKYLHTIEA | |
| | HEWVKHWKYNTSAISWGSLNIQASQPMGLFKVSKLISDLDSTAVYAQ | |
| TuMV protease | GGCSHQS | SEQ ID NO: 129 |
| cleavage site | | |
| PPV protease | MASSKSLFRGLRDYNPIASSICQLNNSMGARQSEMFGLGFGGLIVTNQ | SEQ ID NO: 130 |
| | HLFKRNDGELTIRSHHGEFVVKDTKTLKLLPCKGRDIVIIRLPKDFPPFPRR | |
| | LQFRTPTTEDRVCLIGSNFQTKSISSTMSETSATYPVDNSHFWKHWISTK | |
| | DGHCGLPIVSTRDGSILGLHSLANSTNTQNFYAAFPDNFETTYLSNQDN | |
| | DNWIKQWRYNPDEVCWGSLQLKRDIPQSPFTICKLLTDLDGEFVYTQ | |
| PPV protease | QVVVHQSK | SEQ ID NO: 131 |
| cleavage site | | |
| PVY protease | MASAKSLMRGLRDFNPIAQTVCRLKVSVEYGASEMYGFGFGAYIVANH | SEQ ID NO: 132 |
| | HLFRSYNGSMEVQSMHGTFRVKNLHSLSVLPIKGRDIILIKMPKDFPVFP | |
| | QKLHFRAPTQNERICLVGTNFQEKYASSIITETSTTYNIPGSTFWKHWIET | |
| | DNGHCGLPVVSTADGCIVGIHSLANNAHTTNYYSAFDEDFESKYLRTNE | |
| | HNEWVKSWVYNPDTVLWGPLKLKDSTPKGLFKTTKLVQDLIDHDVVV | |
| | EQ | |
| PVY protease | YDVRHQSR | SEQ ID NO: 133 |
| cleavage site | | |
| ZIKV protease | MASDMYIERAGDITWEKDAEVTGNSPRLDVALDEMGDFSLVEEDGPP | SEQ ID NO: 134 |
| | MREGGGGMGGGGMGALWDVPAPKEVKKGETTDGVYRVMTRRLLG | |
| | STQVGVGVMQEGVFHTMWHVTKGAALRMGEGRLDPYWGDVKQDL | |
| | VSYCGPWKLDAAWDGLSEVQLLAVPPGERARNIQTLPGIFKTKDGDIG | |
| | AVALDYPAGTMGSPILDKCGRVIGLYGNGVVIKNGSYVSAITQGKREEE | |
| | TPVECFE | |
| ZIKV protease | KERKRRGA | SEQ ID NO: 135 |
| cleavage site | | |
| WNV protease | MASSTDMWIERTADISWESDAEITGSSERVDVRLDDDGNFQLMNDPG | SEQ ID NO: 136 |
| | APWKGGGGMGGGGGVLWDTPSPKEYKKGDTTTGVYRIMTRGLLGSY | |
| | QAGAGVMVEGVFHTLWHTTKGAALMMGEGRLDPYWGSVKEDRLCY | |
| | GGPWKLQHKWNGQDEVQMIVVEPGKNVKNVQTKPGVFKTPEGEIG | |
| | AVTLDFPTGTMGSPIVDKNGDVIGLYGNGVIMPNGSYISAIVQGERMD | |
| | EPIPAGFEPEML | |
| WNV protease | KQKKRGGK | SEQ ID NO: 137 |
| cleavage site | | |

In some embodiments, the protease is a TEV protease, a TuMV protease, a PPV protease, a PVY protease, a ZIKV protease, or a WNV protease.

In some embodiments, the protease cleavage site is a self-cleaving peptide, such as the 2A peptides. “2A peptides” are 18-22 amino-acid-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-12A), and T2A (those a asigna virus 2A) were also identified. A few non-limiting examples of 2A peptides are provided in SEQ ID NOs: 138-140.

In some embodiments, the protease is a TEV protease. In some embodiments, the TEV protease comprises a sequence as set forth in SEQ ID NO: 124.

In some embodiments, the first and/or the second TEV protease fragment is not able to cleave the TEV cleavage site on its own. However, in the presence of the remaining portion of the TEV protease, this fragment will be able to effectuate the cleavage. The TEV fragment may be the TEV N-terminal domain (e.g., SEQ ID NO: 125) or the TEV C-terminal domain (e.g., SEQ ID NO: 126). In some embodiments, the first TEV protease fragment comprises a sequence of SEQ ID NO: 125. In some embodiments, the first TEV protease fragment comprises a sequence of SEQ ID NO: 126.

A “nucleobase deaminase inhibitor” or an “inhibitory domain” refers to a protein or a protein domain that inhibits the deaminase activity of a nucleobase deaminase.

In some embodiments, the nucleobase deaminase inhibitor is an inhibitory domain of a nucleobase deaminase.

In some embodiments, the nucleobase deaminase inhibitor is an inhibitory domain of a cytidine deaminase. In some embodiments, the nucleobase deaminase inhibitor is the mouse APOBEC3 cytidine deaminase domain 2 (mA3-CDA2, SEQ ID NO: 141). In some embodiments, the nucleobase deaminase inhibitor is the human APOBEC3B cytidine deaminase domain 1 (hA3B-CDA1, SEQ ID NO: 142).

Table 7 shows 44 proteins/domains that have significant sequence homology to mA3-CDA2 core sequence and Table 8 shows 43 proteins/domains that have significant sequence homology to hA3B-CDA1. All of these proteins and domains, as well as their variants and equivalents, are contemplated to have nucleobase deaminase inhibition activities.

TABLE 7

| Name | Sequence | SEQ ID NO: |
|---|---|---|
| Mouse APOBEC3 | SEKGKQHAEILFLDKIRSMELSQVTITCYLTWSPC | SEQ ID NO: 156 |
| cytidine deaminase | PNCAWQLAAFKRDRPDLILHIYTSRLYFHWKRPF | |
| domain 2 core | QKGLC | |
| (AA282-AA355) | | |
| Mus spicilegus A3 | SEKGKQHAEILFLDKIRSMELSQVTITCYLTWSPC | SEQ ID NO: 157 |
| (AA248-AA321) | PNCAWQLAAFKRDRPDLIPHIYTSRLYFHWKRPF | |
| | QKGLC | |
| Cricetulus | SEKGKQHAEILFLDKIRSMELSQVTITCYLTWSPC | SEQ ID NO: 158 |
| longicaudatus A3 | PNCAWRLAAFKRDRPDLILHIYTSRLYFHWKRPF | |
| (AA249-AA322) | QKGLC | |
| Mus terricolor A3 | SEKGKQHAEILFLNKIRSMELSQVTITCYLTWSPC | SEQ ID NO: 159 |
| (AA248-AA321) | PNCAWQLAAFKKDRPDLILHIYTSRLYFHWKRPF | |
| | QKGLC | |
| Mus caroli A3 | SKKGKQHAEILFLDKIRSMELSQVTITCYLTWSPC | SEQ ID NO: 160 |
| (AA260-AA333) | PNCAWQLAAFKRDHPDLILHIYTSRLYFHWKRPF | |
| | QKGLC | |
| Mus pahari A3 | SKKGKQHAEILFLEKIRSMELSQMRITCYLTWSPC | SEQ ID NO: 161 |
| (AA263-AA336) | PNCAWQLAAFQKDRPDLILHIYTSRLYFHWRRIFQ | |
| | KGLC | |
| Mus shortridgei A3 | SKKGKQHAEILFLEKIRSMELSQMRITCYLTWSPC | SEQ ID NO: 162 |
| (AA233-AA306) | PNCAWQLAAFQKDRPDLILHIYTSRLYFHWRRIFQ | |
| | KGLC | |
| Mus setulosus A3 | SKKGKQHAEILFLDKIRSMELSQVRITCYLTWSPC | SEQ ID NO: 163 |
| (AA29-AA302) | PNCAWQLETFKKDRPDLILHIYTSRLYFHWKRAF | |
| | QEGLC | |
| Grammomys | SKKGKPHAEILFLDKMWSMEELSQVRITCYLTWS | SEQ ID NO: 164 |
| surdaster A3 | PCPNCARQLAAFKKDHPGLILRIYTSRLYFYWRR | |
| (AA270-AA344) | KFQKGLC | |
| Rattus norvegicus | KKGEQHVEILFLEKMRSMELSQVRITCYLTWSPCP | SEQ ID NO: 165 |
| A3 (AA256- | NCARQLAAFKKDHPDLILRIYTSRLYFYWRKKFQ | |
| AA328) | KGLC | |
| Mastomys coucha | SKKGRQHAEILFLEKVRSMQLSQVRITCYLTWSPC | SEQ ID NO: 166 |
| A3 (AA258- | PNCAWQLAAFKMDHPDLILRIYASRLYFHWRRAF | |
| AA331) | QKGLC | |
| Cricetulus griseus | NKKGKHAEILFIDEMRSLELGQVQITCYLTWSPCP | SEQ ID NO: 167 |
| A3B (AA235- | NCAQELAAFKSDHPDLVLRIYTSRLYFHWRRKYQ | |
| AA307) | EGLC | |
| Peromyscus | NKKGKHAEILFIDEMRSLELGQARITCYLTWSPCP | SEQ ID NO: 168 |
| leucopus A3 | NCAQKLAAFKKDHPDLVLRVYTSRLYFHWRRKY | |
| (AA266-AA338) | QEGLC | |
| Mesocricetus | NKKDKHAEILFIDKMRSLELCQVRITCYLTWSPCP | SEQ ID NO: 169 |
| auratus A3 | NCAQELAAFKKDHPDLVLRIYTSRLYFHWRRKY | |
| (AA268-AA340) | QEGLC | |
| Microtus | NKKGKHAEILFIDEMRSLKLSQERITCYLTWSPCP | SEQ ID NO: 170 |
| ochrogaster A3B | NCAQELAAFKRDHPGLVLRIYASRLYFHWRRKY | |
| (AA266-AA338) | QEGLC | |
| Nannospalax galili | NKRAKHAEILLIDMMRSMELGQVQITCYITWSPC | SEQ ID NO: 171 |
| A3 (AA231- | PTCAQELAAFKQDHPDLVLRIYASRLYFHWKRKF | |
| AA302) | QKGL | |
| Meriones | NKKGRHAEICLIDEMRSLGLGKAQITCYLTWSPC | SEQ ID NO: 172 |
| unguiculatus A3 | RKCAQELATFKKDHPDLVLRVYASRLYFHWSRK | |
| (AA233-AA305) | YQQGLC | |
| Dipodomys ordii | NKKGHHAEIRFIERIRSMGLDPSQDYQITCYLTWS | SEQ ID NO: 173 |
| A3 (AA256- | PCLDCAFKLAKLKKDFPRLTLRIFTSRLYFHWIRK | |
| AA330) | FQKGL | |
| Jaculus jaculus A3 | NKKGKHAEARFVDKMRSMQLDHALITCYLTWSP | SEQ ID NO: 174 |
| (AA303-AA374) | CLDCSQKLAALKRDHPGLTLRIFTSRLYFHWVKK | |
| | FQEGL | |
| Chinchilla lanigera | SPQKGHHAESRFIKRISSMDLDRSRSYQITCFLTWS | SEQ ID NO: 175 |
| A3H (AA86- | PCPSCAQELASFKRAHPHLRFQIFVSRLYFHWKRS | |
| AA161) | YQAGL | |
| Heterocephalus | KKGYHAESRFIKRICSMDLGQDQSYQVTCFLTWS | SEQ ID NO: 176 |
| glaber A3 (AA277- | PCPHCAQELVSFKRAHPHLRLQIFTARLFFHWKRS | |
| AA350) | YQEGL | |
| Octodon degus A3 | KKGQHAEIRFIERIHSMALDQARSYQITCFLTWSP | SEQ ID NO: 177 |
| (AA256-AA329) | CPFCAQELASFKSTHPRVHLQIFVSRLYFHWKRSY | |
| | QEGL | |
| Urocitellus parryii | NKKGHHAEIRFIKKIRSLDLDQSQNYEVTCYLTWS | SEQ ID NO: 178 |
| A3 (AA256- | PCPDCAQELVALTRSHPHVRLRLFTSRLYFHWFW | |
| AA330) | SFQEGL | |
| Aotus nancymaae | NRHAEICFIDEIESMGLDKTQCYEVTCYLTWSPCP | SEQ ID NO: 179 |
| A3H (AA75- | SCAQKLAAFTKAQVHLNLRIFASRLYYHWRSSYQ | |
| AA146) | KGL | |
| Cebus capucinus | NRHAEICFIDEIESMGLDKTQCYEVTCYLTWSPCP | SEQ ID NO: 180 |
| imitator A3H | SCAQKLVAFAKAQDHLNLRIFASRLYYHWRRRY | |
| (AA55-AA126) | KEGL | |
| Saimiri boliviensis | HVEICFIDKIASMELDKTQCYDVTCYLTWSPCPSC | SEQ ID NO: 181 |
| boliviensis A3H | AQKLAAFAKAQDHLNLRIFASRLYYHWRRSYQK | |
| (AA56-AA125) | GL | |
| Homo sapiens A3H | NKKKCHAEICFINEIKSMGLDETQCYQVTCYLTW | SEQ ID NO: 182 |
| (AA49-AA123) | SPCSSCAWELVDFIKAHDHLNLGIFASRLYYHWC | |
| | KPQQKGL | |
| Homo sapiens | ENKKKCHAEICFINEIKSMGLDETQCYQVTCYLT | SEQ ID NO: 183 |
| ARP10 (AA48- | WSPCSSCAWELVDFIKAHDHLNLGIFASRLYYHW | |
| AA123) | CKPQQKGL | |
| Pan paniscus A3H | NKKKCHAEICFINEIKSMGLDETQCYQVTCYLTW | SEQ ID NO: 184 |
| (AA49-AA123) | SPCSSCAWKLVDFIQAHDHLNLRIFASRLYYHWC | |
| | KPQQEGL | |
| Symphalangus | NKKKRHAEIRFINKIKSMGLDETQCYQVTCYLTW | SEQ ID NO: 185 |
| syndactylus A3H | SPCPSCAWELVDFIKAHDHLNLGIFASRLYYHWC | |
| (AA49-AA123) | RHQQEGL | |
| Macaca mulatta | NKKKDHAEIRFINKIKSMGLDETQCYQVTCYLTW | SEQ ID NO: 186 |
| A3H (AA49- | SPCPSCAGELVDFIKAHRHLNLRIFASRLYYHWRP | |
| AA123) | NYQEGL | |
| Theropithecus | NKKKEHAEIRFINKIKSMGLDETQCYQVTCYLTW | SEQ ID NO: 187 |
| gelada A3H | SPCPSCAGKLVDFIKAHHHLNLRIFASRLYYHWRP | |
| (AA54-AA128) | NYQEGL | |
| Mandrillus | NKKKHHAEIHFINKIKSMGLDETQCYQVTCYLTW | SEQ ID NO: 188 |
| leucophaeus A3H | SPCPSCARELVDFIKAHRHLNLRIFASRLYYHWRP | |
| (AA49-AA123) | HYQEGL | |
| Bos grunniens A3 | NKKQRHAEIRFIDKINSLDLNPSQSYKIICYITWSP | SEQ ID NO: 189 |
| (AA74-AA148) | CPNCANELVNFITRNNHLKLEIFASRLYFHWIKPF | |
| | KMGL | |
| Bubalus bubalis A3 | NKKQRHAEIRFIDKINSLDLNPSQSYKIICYITWSP | SEQ ID NO: 190 |
| (AA74-AA148) | CPNCASELVDFITRNDHLDLQIFASRLYFHWIKPF | |
| | KRGL | |
| Odocoileus | NKKQRHAEIRFIDKINSLNLDRRQSYKIICYITWSP | SEQ ID NO: 191 |
| virginianus texanus | CPRCASELVDFITGNDHLNLQIFASRLYFHWKKPF | |
| A3H (AA209-AA283) | QRGL | |
| Sus scrofa A3 | NKKKRHAEIRFIDKINSLNLDQNQCYRIICYVTWS | SEQ ID NO: 192 |
| (AA51-AA125) | PCHNCAKELVDFISNRHHLSLQLFASRLYFHWVR | |
| | CYQRGL | |
| Ceratotherium | NKKKRHAEIRFIDKIKSLGLDRVQSYEITCYITWSP | SEQ ID NO: 193 |
| simum simum A3B | CPTCALELVAFTRDYPRLSLQIFASRLYFHWRRRS | |
| (AA232-AA306) | IQGL | |
| Equus caballus | NKKKRHAEIRFIDKINSLGLDQDQSYEITCYVTWS | SEQ ID NO: 194 |
| A3H (AA79- | PCATCACKLIKFTRKFPNLSLRIFVSRLYYHWFRQ | |
| AA153) | NQQGL | |
| Enhydra lutris | KKKRHAEIRFIDSIRALQLDQSQRFEITCYLTWSPC | SEQ ID NO: 195 |
| kenyoni A3B | PTCAKELAMFVQDHPHISLRLFASRLYFHWRWKY | |
| (AA243-AA316) | QEGL | |
| Leptonychotes | KKKRHAEIRFIDNIKALRLDTSQRFEITCYVTWSPC | SEQ ID NO: 196 |
| weddellii A3H | PTCAKELVAFVRDHRHISLRLFASRLYFHWLREN | |
| (AA50-AA123) | KKGL | |
| Ursus arctos | NKKKRHAEIRFIDKIRSLQRDSSQTFEITCYVTWSP | SEQ ID NO: 197 |
| horribilis A3F | CFTCAEELVAFVRDHPHVRLRLFASRLYFHWLRK | |
| (AA552-AA626) | YQEGL | |
| Panthera leo | NKKKRHAEICFIDKIKSLTRDTSQRFEIICYITWSPC | SEQ ID NO: 198 |
| bleyenberghi A3H | PFCAEELVAFVKDNPHLSLRIFASRLYVHWRWKY | |
| (AA50-AA124) | QQGL | |
| Panthera tigris | NKKKRHAEICFIDKIKSLTRDTSQRFEIICYITWSPC | SEQ ID NO: 199 |
| sumatrae A3H | PFCAEELVAFVKDNPHLSLRIFASRLYVHWRWKY | |
| (AA50-AA124) | QQGL | |
| Tupaia belangeri | NKKHRHAEVRFIAKIRSMSLDLDQKHQLTCYLTW | SEQ ID NO: 200 |
| A3 (AA46-AA120) | SPCPSCAQELVTFMAESRHLNLQVFVSRLYFHWQ | |
| | RDFQQGL | |

TABLE 8

| Name | Sequence | SEQ ID NO: |
|---|---|---|
| Gorilla A3B | GRSYNWLCYEVKIKRGRSNLLWNTGVFRGQMYS | SEQ ID NO: 201 |
| (AA29-AA138) | QPEHHAEMCFLSWFCGNQLPAYKCFQITWFVSW | |
| | TPCPDCVAKLAEFLAEYPNVTLTISTARLYYYWE | |
| | RDYRRALCRL | |
| Pan paniscus A3B | GRSYTWLCYEVKIRRGHSNLLWDTGVFRGQMYS | SEQ ID NO: 202 |
| (AA29-AA138) | QPEHHAEMYFLSWFCGNQLPAYKCFQITWFVSW | |
| | TPCPDCVAKLAEFLAEHPNVTLTISAARLYYYWE | |
| | RDYRRALCRL | |
| Pan troglodytes | GRSYTWLCYEVKIRRGHSNLLWDTGVFRGQMYS | SEQ ID NO: 203 |
| A3B (AA29- | QPEHHAEMCFLSWFCGNQLSAYKCFQITWFVSW | |
| AA138) | TPCPDCVAKLAKFLAEHPNVTLTISAARLYYYWE | |
| | RDYRRALCRL | |
| Gorilla A3F | RNTVWLCYEVKTKGPSRPPLDAKIFRGQVYFEPQ | SEQ ID NO: 204 |
| (AA30-AA137) | YHAEMCFLSWFCGNQLPAYKCFQITWFVSWTPCP | |
| | DCVAKLAEFLAEHPNVTLTISAARLYYYWE | |
| Pan troglodytes | RNTVWLCYEVKTKGPSRPRLDTKIFRGQVYFEPQ | SEQ ID NO: 205 |
| A3F (AA30- | YHAEMCFLSWFCGNQLPAYKCFQITWFVSWTPCP | |
| AA137) | DCVAKLAEFLAEHPNVTLTISAARLYYYWERDYR | |
| | RALCRL | |
| Human sapiens | RNTVWLCYEVKTKGPSRPRLDAKIFRGQVYSQPE | SEQ ID NO: 206 |
| A3F (AA30- | HHAEMCFLSWFCGNQLPAYKCFQITWFVSWTPCP | |
| AA137) | DCVAKLAEFLAEHPNVTLTISAARLYYYWERDYR | |
| | RALCRL | |
| Macaca leonine | RNTVWLCYEVKTRGPSMPTWGTKIFRGQVCFEPQ | SEQ ID NO: 207 |
| A3F (AA30- | YHAEMCFLSRFCGNQLPAYKRFQITWFVSWTPCP | |
| AA137) | DCVAKVAEFLAEHPNVTLTISAARLYYYWETDYR | |
| | RALCRL | |
| Macaca nemestrina | RNTVWLCYEVKTRGPSMPTWGTKIFRGQVCFEPQ | SEQ ID NO: 208 |
| A3F (AA30- | YHAEMCFLSRFCGNQLPAYKRFQITWFVSWTPCP | |
| AA137) | DCVAKVAEFLAEHPNVTLTISAARLYYYWETDYR | |
| | RALCRL | |
| Rhinopithecus | RNTVWLCYEVKTRGPSMPTWGAKIFRGQVYFEP | SEQ ID NO: 209 |
| roxellana A3F | QYHAEMCFLSWFCGNQLPAYKRFQITWFVSWTP | |
| (AA30-AA137) | CPDCVAKVAEFLAEHPNVTLTISAARLYYYWETD | |
| | YRRALCRL | |
| Mandrillus | RNTVWLCYKVKTRGPSMPTWGTKIFRGQVYFQP | SEQ ID NO: 210 |
| leucophaeus A3F | QYHAEMCFLSWFCGNQLPAYKRFQITWFVSWTP | |
| (AA30-AA130) | CPDCVVKVAEFLAEHPNVTLTISAARLYYYWETD | |
| | Y | |
| Macaca mulatta | RNTVWLCYEVKTRGPSMPTWDTKIFRGQVYSKP | SEQ ID NO: 211 |
| A3F (AA30- | EHHAEMCFLSRFCGNQLPAYKRFQITWFVSWTPC | |
| AA137) | PDCVAKVAEFLAEHPNVTLTISAARLYYYWETDY | |
| | RRALCRL | |
| Theropithecus | RNTVWLCYEVKTRGPSMPTWGTKIFRGQVYFQP | SEQ ID NO: 212 |
| gelada A3F (AA30- | QYHAEMCFLSRFCGNQLPAYKRFQITWFVSWNPC | |
| AA137) | PDCVAKVIEFLAEHPNVTLTISAARLYYYWGRDW | |
| | RRALRRL | |
| Cercocebus atys | GRSYTWLCYEVKIRKDPSKLPWYTGVFRGQVYS | SEQ ID NO: 213 |
| A3B (AA29- | KPEHHAEMCFLSRFCGNQLPAYKRFQITWFVSWN | |
| AA138) | PCPDCVAKVIEFLAEHPNVTLTISAARLYYYWSRD | |
| | WQRALCRL | |
| Macaca fascicularis | GRSYTWLCYEVKIRKDPSKLPWDTGVFRGQVYS | SEQ ID NO: 214 |
| A3B (AA29- | KPEHHAEMCFLSRFCGNQLPAYKRFQITWFVSWN | |
| AA138) | PCPDCVAKVIEFLAEHPNVTLTISTARLYYYWGR | |
| | DWQRALCRL | |
| Macaca mulatta | GRSYTWLCYEVKIRKDPSKLPWDTGVFRGQVYS | SEQ ID NO: 215 |
| A3B (AA29- | KPEHHAEMCFLSRFCGNQLPAYKRFQITWFVSWN | |
| AA138) | PCPDCVAKVIEFLAEHPNVTLTISTARLYYYWGR | |
| | DWQRALCRL | |
| Macaca leonina | GRSYTWLCYEVKIRKDPSKLPWDTGVFRGQVYS | SEQ ID NO: 216 |
| A3B (AA29- | KPEHHAEMCFLSRFCGNQLPAYKRFQITWFVSWN | |
| AA138) | PCPDCVVKVIEFLAEHPNVTLTISTARLYYYWGR | |
| | DWQRALCRL | |
| Mandrillus | GRSYTWLCYEVKIRKDPSKLPWYTGVFRGQVYS | SEQ ID NO: 217 |
| leucophaeus A3B | KPEHHAEMCFLSRFCGNQLPAYKRFQITWFVSWN | |
| (AA29-AA138) | PCPDCVAKVIEFLAEHPNVTLTIFTARLYYYWGR | |
| | DWQRALCRL | |
| Macaca nemestrina | GRSYTWLCYEVKIRKDPSKLPWDTGVFRGQVYS | SEQ ID NO: 218 |
| A3B (AA29- | KPEHHAEMCFLSRFCGNQLPAYKRFQITWFVSWN | |
| AA138) | PCPDCVAKVTEFLAEHPNVTLTISTARLYYYWGR | |
| | DWQRALCRL | |
| Rhinopithecus bieti | GRSYTWLCYEVKIRKDPSKLPWDTGVFRGQVYSE | SEQ ID NO: 219 |
| A3F (AA29- | PEHHAEMYFLSWFCGNQLPAYKRFQITWFVSWTP | |
| AA138) | CPDCVAKVAEFLTEHPNVTLTISAARLYYYRGRD | |
| | WRRALCRL | |
| Rhinopithecus | GRSYTWLCYEVKIRKDPSKLPWDTGVFRGQVYSE | SEQ ID NO: 220 |
| roxellana A3B | PEHHAEMYFLSWFCGNQLPAYKRFQITWFVSWTP | |
| (AA29-AA138) | CPDCVAKVAEFLTEHPNVTLTISAARLYYYRGRD | |
| | WRRALCRL | |
| Chlorocebus | GRSYTWLCYEVKIRKDPSKLPWDTGVFRGQMYS | SEQ ID NO: 221 |
| sabaeus A3B | KPEHHAEMCFLSWFCGNQLPAHKRFQITWFVSW | |
| (AA29-AA138) | TPCPDCVAKVAEFLAEYPNVTLTISAARLYYYWE | |
| | TDYRRALCRL | |
| Nomascus | RSYTWLCYEVKIRKDPSKLPWDTGVFRGQMYFQ | SEQ ID NO: 222 |
| leucogenys A3B | PEYHAEMCFLSWFCGNQLPAYKRFQITWFVSWTP | |
| (AA30-AA138) | CPDCVAKVAVFLAEHPNVTLTISAARLYYYWEK | |
| | DWQRALCRL | |
| Cercocebus atys | GRSYTWLCYEVKIKKYPSKLLWDTGVFQGQVYF | SEQ ID NO: 223 |
| A3F (AA29- | QPQYHAEMCFLSRFCGNQLPAYKRFQITWFVSW | |
| AA138) | NPCPDCVAKVTEFLAEHPNVTLTISAARLYYYWE | |
| | KDXRRALRRL | |
| Papio anubis A3F | GRSYTWLCYEVKIKEDPSKLLWDTGVFQGQVYF | SEQ ID NO: 224 |
| (AA29-AA138) | QPQYHAEMCFLSRFCGNQLPAYKRFQITWFVSW | |
| | NPCPDCVAKVTEFLAEHPNVTLTISAARLYYYWG | |
| | RDWRRALRRL | |
| Chlorocebus | GRRYTWLCYEVKIKKDPSKLPWDTGVFPGQVRP | SEQ ID NO: 225 |
| aethiops A3D | KFQSNRRYEVYFQPQYHAEMYFLSWFCGNQLPA | |
| (AA29-AA150) | YKHFQITWFVSWNPCPDCVAKVTEFLAEHRNVTL | |
| | TISAARLYYYWGKDWRRALCRL | |
| Chlorocebus | GRRYTWLCYEVKIKKDPSKLPWDTGVFPGQPQY | SEQ ID NO: 226 |
| sabaeus A3D | HAEMYFLSWFCGNQLPAYKHFQITWFVSWNPCP | |
| (AA29-AA134) | DCVAKVTEFLAEHRNVTLTISAARLYYYWGKDW | |
| | RRALCRL | |
| Chlorocebus | GRRYTWLCYEVKIKKDPSKLPWDTGVFPGQVRP | SEQ ID NO: 227 |
| sabaeus A3F | KFQSNRRQKVYFQPQYHAEMYFLSWFCGNQLPA | |
| (AA29-AA150) | YKHFQITWFVSWNPCPDCVAKVTEFLAEHRNVTL | |
| | TISAARLYYYWGKDWRRALCRL | |
| Erythrocebus patas | GRRYTWLCYEVKIKKDPSKLPWDTGVFQGQVRP | SEQ ID NO: 228 |
| A3D (AA29- | KFQSNRRYEVYFQPQYHAEMCFLSWFCGNQLPA | |
| AA150) | YKHFQITWFVSWNPCPDCVAKVTEFLAEHPNVTL | |
| | TISAARLYYYWGKDWRRALCRL | |
| Macaca fascicularis | GRSYTWLCYEVKIRKDPSKLPWDTGVFRGQVRP | SEQ ID NO: 229 |
| A3D (AA29- | KLQSNRRYELSNWECRKRVYFQPQYHAEMYFLS | |
| AA159) | WFCGNQLPANKRFQITWFASWNPCPDCVAKVTE | |
| | FLAEHPNVTLTISVARLYYYRGKDWRRALRRL | |
| Macaca fascicularis | GRSYTWLCYEVKIRKDPSKLPWDTGVFRGQVYF | SEQ ID NO: 230 |
| A3F (AA29- | QPQYHAEMYFLSWFCGNQLPANKRFQITWFASW | |
| AA138) | NPCPDCVAKVTEFLAEHPNVTLTISVARLYYYRG | |
| | KDWRRALRRL | |
| Macaca nemestrina | GRSYTWLCYEVKIRKDPSKLPWDTGVFRDQVYF | SEQ ID NO: 231 |
| A3D (AA29- | QPQYHAEMCFLSWFCGNQLPANKRFQITWFVSW | |
| AA138) | NPCPDCVTKVTEFLAEHPNVTLTISVARLYYYRG | |
| | KDWRRALRRL | |
| Macaca leonina | GRSYTWLCYEVKIRKDPSKLPWYTGVFRGQVYF | SEQ ID NO: 232 |
| A3D (AA29- | QPQYHAEMCFLSWFCGNQLPANKRFQITWFVSW | |
| AA138) | NPCPDCVAKVTEFLAEHPNVTLTISVARLYYYRG | |
| | KDWRRALRRL | |
| Macaca mulatta | GRSYTWLCYEVKIRKDPSKLPWDTGVFRGQVYF | SEQ ID NO: 233 |
| A3D (AA29- | QPQYHAEMCFLSWFCGNQLPAYKRFQITWFVSW | |
| AA138) | NPCPDCVAKVTEFLAEHPNVTLTISVARLYYYRG | |
| | KDWRRALCRL | |
| Gorilla A3D | GRSYTWLCYEVKIRRGSSNLLWNTGVFRGPVPPK | SEQ ID NO: 234 |
| (AA29-AA150) | LQSNHRQEVYFQFENHAEMCFLSWFCGNRLPAN | |
| | RRFQITWFVSWNPCLPCVVKVTKFLAEHPNVTLTI | |
| | SAARLYYYRDREWRRVLRRL | |
| Pan paniscus A3D | GRSYTWLCYEVKIKRGCSNLIWDTGVFRGPVLPK | SEQ ID NO: 235 |
| (AA29-AA150) | LQSNHRQEVYFQFENHAEMCFFSWFCGNRLPAN | |
| | RRFQITWFVSWNPCLPCVVKVTKFLAEHPNVTLTI | |
| | SAARLYYYQDREWRRVLRRL | |
| Pan troglodytes | GRSYTWLCYEVKIKRGCSNLIWDTGVFRGPVLPK | SEQ ID NO: 236 |
| A3D (AA29- | LQSNHRQEVYFQFENHAEMCFFSWFCGNRLPAN | |
| AA150) | RRFQITWFVSWNPCLPCVVKVTKFLAEHPNVTLTI | |
| | SAARLYYYQDREWRRVLRRL | |
| Homo sapiens A3D | GRSYTWLCYEVKIKRGRSNLLWDTGVFRGPVLPK | SEQ ID NO: 237 |
| (AA29-AA150) | RQSNHRQEVYFRFENHAEMCFLSWFCGNRLPAN | |
| | RRFQITWFVSWNPCLPCVVKVTKFLAEHPNVTLTI | |
| | SAARLYYYRDRDWRWVLLRL | |
| Nomascus | GRSYTWLCYEVKIRKDPSKLPWDKGVFRGQVLP | SEQ ID NO: 238 |
| leucogenys A3D | KFQSNHRQEVYFQLENHAEMCFLSWFCGNQLPA | |
| (AA29-AA150) | NRRFQITWFVSWNPCLPCVAKVTEFLAEHPNVTL | |
| | TISAARLYYYRGRDWRRALRRL | |
| Saimiri boliviensis | GKKYTWLCYEVKIKKDTSKLPWNTGVFRGQVNF | SEQ ID NO: 239 |
| A3C (AA29- | NPEHHAEMYFLSWFRGKLLPACKRSQITWFVSW | |
| AA138) | NPCLYCVAKVAEFLAEHPNVTLTVSTARLYCYW | |
| | KKDWRRALRKL | |
| Saimiri boliviensis | GKKYTWLCYEVKIKKDTSKLPWNTGVFRGQVNF | SEQ ID NO: 240 |
| A3F (AA29- | NPEHHAEMYFLSWFRGKLLPACKRSQITWFVSW | |
| AA138) | NPCLYCVAKVAEFLAEHPNVTLTVSTARLYCYW | |
| | KKDWRRALRKL | |
| Piliocolobus | GRRYTWLCYEVKIMKDHSKLPWYTGVFRGQVYF | SEQ ID NO: 241 |
| tephrosceles A3F | EPQNHAEMCFLSWFCGNQLPAYECCQITWFVSW | |
| (AA36-AA145) | TPCPDCVAKVTEFLAEHPNVTLTISAARLYYYRG | |
| | RDWRRALRRL | |
| Colobus angolensis | GRRYTWLCYEVKISKDPSKLPWDTGIFRGQVYFE | SEQ ID NO: 242 |
| palliatus A3F | PQYHAEMCFLSWYCGNQLPAYKCFQITWFVSWT | |
| (AA29-AA138) | PCPDCVGKVAEFLAEHPNVTLTISAARLYYYWET | |
| | DYRRALCRL | |
| Pongo abelii A3F | RNYTWLCYEVKIRKDPSKLAWDTGVFRGQVLPK | SEQ ID NO: 243 |
| (AA30-AA150) | LQSNHRREVYFEPQYHAEMCFLSWFCGNQLSAY | |
| | ERFQITWFVSWTPCPDCVAMLAEFLAEHPNVTLT | |
| | VSAARLYYYWERDYRGALRRL | |

In some embodiments, the inhibitory domain of a cytidine deaminase comprises an amino acid sequence as set forth in SEQ ID NO: 141 or SEQ ID NO: 142.

The term “nucleobase deaminase” as used herein, refers to a group of enzymes that catalyze the hydrolytic deamination of nucleobases such as cytidine, deoxycytidine, adenosine and deoxyadenosine. Non-limiting examples of nucleobase deaminases include cytidine deaminases and adenosine deaminases.

Some of the nucleobase deaminases have a single, catalytic domain, while others also have other domains, such as an inhibitory domain as described in WO2020156575A1 In some embodiments, therefore, the gene editing system disclosed herein only includes the catalytic domain, such as mouse A3 cytidine deaminase domain (mA3-CDA1, SEQ ID NO: 143) and human A3B cytidine deaminase domain 2 (hA3B-CDA2, SEQ ID NO: 144). In some embodiments, the gene editing system disclosed herein includes at least a catalytic core of the catalytic domain. For instance, when mA3-CDA1 was truncated at residues 196/197 the CDA1 domain still retained substantial editing efficiencies.

In some embodiments, the nucleotide deaminase is a cytidine deaminase. In some embodiments, the nucleotide deaminase is a cytidine deaminase comprising an amino acid sequence of SEQ ID NO: 143. In some embodiments, the nucleotide deaminase is a cytidine deaminase comprising an amino acid sequence of SEQ ID NO: 144.

TABLE 9

| | | |
|---|---|---|
| mouse | MSSSTLSNICLTKGLPETRFWVEGRRMDPLSEEEFY | SEQ ID NO: |
| APOBEC3 | SQFYNQRVKHLCYYHRMKPYLCYQLEQFNGQAPL | 141 |
| cytidine | KGCLLSEKGKQHAEILFLDKIRSMELSQVTITCYLT | |
| deaminase | WSPCPNCAWQLAAFKRDRPDLILHIYTSRLYFHW | |
| domain 2 | KRPFQKGLCSLWQMGILVDVMDLPQFTDCWTNF | |
| (mA3-CDA2) | VNPKRPFWPWKGLEIISRRTQRRLRRIKESWGLQ | |
| | DLVNDFGNLQLGPPMS | |
| human | MNPQIRNPMERMYRDTFYDNFENEPILYGRSYT | SEQ ID NO: |
| APOBEC3B | WLCYEVKIKRGRSNLLWDTGVFRGQVYFKPQY | 142 |
| cytidine | HAEMCFLSWFCGNQLPAYKCFQITWFVSWTPCP | |
| deaminase | DCVAKLAEFLSEHPNVTLTISAARLYYYWERDY | |
| domain 1 | RRALCRLSQAGARVKIMDYEEFAYCWENFVYN | |
| (hA3B- | EGQ | |
| CDA1) | | |
| mouse | MGPFCLGCSHRKCYSPIRNLISQETFKFHFKNLG | SEQ ID NO: |
| APOBEC3 | YAKGRKDTFLCYEVTRKDCDSPVSLHHGVFKN | 143 |
| cytidine | KDNIHAEICFLYWFHDKVLKVLSPREEFKITWY | |
| deaminase | MSWSPCFECAEQIVRFLATHHNLSLDIFSSRLYN | |
| domain 1 | VQDPETQQNLCRLVQEGAQVAAMDLYEFKKC | |
| (mA3-CDA1) | WKKFVDNGGRRFRPWKRLLTNFRYQDSKLQE | |
| | ILRPCYISVPSS | |
| human | MQFMPWYKFDENYAFLHRTLKEILRYLMDPDT | SEQ ID NO: |
| APOBEC3B | FTFNFNNDPLVLRRRQTYLCYEVERLDNGTWVL | 144 |
| cytidine | MDQHMGFLCNEAKNLLCGFYGRHAELRFLDLV | |
| deaminase | PSLQLDPAQIYRVTWFISWSPCFSWGCAGEVRA | |
| domain 2 | FLQENTHVRLRIFAARIYDYDPLYKEALQMLRD | |
| (hA3B- | AGAQVSIMTYDEFEYCWDTFVYRQGCPFQPWD | |
| CDA2) | GLEEHSQALMGRLRAILQNQGN | |

“Cytidine deaminase” refers to enzymes that catalyze the hydrolytic deamination of cytidine and deoxycytidine to uridine and deoxyuridine, respectively. Cytidine deaminases maintain the cellular pyrimidine pool. A family of cytidine deaminases is APOBEC (“apolipoprotein B mRNA editing enzyme, catalytic polypeptide-like”). Members of this family are C-to-U editing enzymes. Some APOBEC family members have two domains, one domain of APOBEC like proteins is the catalytic domain, while the other domain is a pseudocatalytic domain. More specifically, the catalytic domain is a zinc dependent cytidine deaminase domain and is important for cytidine deamination. RNA editing by APOBEC-1 requires homodimerisation and this complex interacts with RNA binding proteins to form the editosome.

Non-limiting examples of APOBEC proteins include APOBEC1, APOBEC2, APOBEC3A, APOBEC3B, APOBEC3C, APOBEC3D, APOBEC3F, APOBEC3G, APOBEC3H, APOBEC4, and activation-induced (cytidine) deaminase (AID).

Various mutants of the APOBEC proteins are also known that have brought about different editing characteristics for base editors. For instance, for human APOBEC3A, certain mutants (e.g., W98Y, Y130F, Y132D, W104A, D131Y and P134Y) even outperform the wildtype human APOBEC3A in terms of editing efficiency or editing window. Accordingly, the term APOBEC and each of its family member also encompasses variants and mutants that have certain level (e.g., 70%, 75%, 80%, 85%, 90%, 95%, 98%, 99%) of sequence identity to the corresponding wildtype APOBEC protein or the catalytic domain and retain the cytidine deaminating activity. The variants and mutants can be derived with amino acid additions, deletions and/or substitutions. Such substitutions, in some embodiments, are conservative substitutions.

In some embodiments, the cytidine deaminase is selected from the group consisting of APOBEC3B (A3B), APOBEC3C (A3C), APOBEC3D (A3D), APOBEC3F (A3F), APOBEC3G (A3G), APOBEC3H (A3H), APOBECI (A1), APOBEC3 (A3), APOBEC2 (A2), APOBEC4 (A4), and AICDA (AID).

In some embodiments, the cytidine deaminase is a human or mouse cytidine deaminase.

In some embodiments, the catalytic domain of the cytidine deaminase is a mouse A3 cytidine deaminase domain 1 (CDA1) or human A3B cytidine deaminase domain 2 (CDA2).

In some embodiments, the cytidine deaminase comprises an amino acid sequence of any one of SEQ ID NOs: 792-827. (Table 24)

TABLE 24

| Name | Sequence | SEQ ID |
|---|---|---|
| Felis catus | MNPLQEVIFCRQFGNQHRVPKPYYRRKTYLCYQLKLPEGTL | SEQ ID |
| APOBEC3H | IHKDCLRNKKKRHAEMCFIDKIKALTRDTSQRFEIICYITWSP | NO: 792 |
| | CPFCAEELVAFVKDNPHLSLRIFASRLYVHWRWKYQQGLR | |
| | HLHASGIPVAVMSLPEFEDCWRNFVDHQDRSFQPWPNLDQ | |
| | YSKSIKRRLGKILTPLNDLRNDFRNLKLE | |
| Human | MEASPASGPRHLMDPHIFTSNFNNGIGRHKTYLCYEVERLD | SEQ ID |
| APOBEC3A | NGTSVKMDQHRGFLHNQAKNLLCGFYGRHAELRFLDLVPS | NO: 793 |
| | LQLDPAQIYRVTWFISWSPCFSWGCAGEVRAFLQENTHVRL | |
| | RIFAARIYDYDPLYKEALQMLRDAGAQVSIMTYDEFKHCW | |
| | DTFVDHQGCPFQPWDGLDEHSQALSGRLRAILQNQGN | |
| Cricetulus | MGPFCLGCSHRKCYSPIRNLISQETFKFHFKNLGYAKGRKD | SEQ ID |
| longicaudatus | TFLCYEVTRKDCDSPVSLHHGVFKNKGNIHAEVCFLYWFH | NO: 794 |
| A3 | DKVLKVLSPREEFKITWYMSWSPCFECAEQIVRFLATHHYL | |
| | SLDIFSSRLYNVQDPETQQNLCRLVQEGAQVAAMDLYEFKK | |
| | CWKKFVTMVAGDSGLGKRLLTNFRYQDSKLQEILRRMDPL | |
| | SEEEFYSQFYNQRVKHLCYYHRMKPYLCYQLEQFNGQAPL | |
| | KGCLLSEKGKQHAEILFLDKIRSMELSQVTITCYLTWSPCPN | |
| | CAWRLAAFKRDRPDLILHIYTSRLYFHWKRPFQKGLCSLWQ | |
| | SGILVDVMDLPQFTDCWTNFVNPKRPFWPWKGLEIISRRTQ | |
| | RRLRRIKESWGLQDLVNDFGNLQLGPPMS | |
| Rattus | MGPFCLGCSHRKCYSPIRNLISQETFKFHFKNLRYAIDRKDT | SEQ ID |
| norvegicus | FLCYEVTRKDCDSPVSLHHGVFKNKDNIHAEICFLYWFHDK | NO: 795 |
| APOBEC3 | VLKVLSPREEFKITWYMSWSPCFECAEQVLRFLATHHNLSL | |
| | DIFSSRLYNIRDPENQQNLCRLVQEGAQVAAMDLYEFKKC | |
| | WKKFVDNGGRRFRPWKKLLTNFRYQDSKLQEILRPCYIPVP | |
| | SSSSSTLSNICLTKGLPETRFCVERRRVHLLSEEEFYSQFYNQ | |
| | RVKHLCYYHGVKPYLCYQLEQFNGQAPLKGCLLSEKGKQH | |
| | AEILFLDKIRSMELSQVIITCYLTWSPCPNCAWQLAAFKRDR | |
| | PDLILHIYTSRLYFHWKRPFQKGLCSLWQSGILVDVMDLPQF | |
| | TDCWTNFVNPKRPFWPWKGLEIISRRTQRRLHRIKESWGLQ | |
| | DLVNDFGNLQLGPPMS | |
| Pongo | MALLTAKTFSLQFNNKRRIKRPYYPRKALLCYQLTPQNGST | SEQ ID |
| pygmaeus | PTRGYFKNKKKCHAEIRFINEIKSMGLDETQCYQVTCYLTW | NO: 796 |
| APOBEC3H | SPCPSCVRELVAFIKAHDHLNLRIFASRLYCHWCRRQQEGL | |
| | RLLCGSQVPVEVMGSREFADCWENFVDHEKPLSFNPSEMLE | |
| | ELDKNSRAIKRRLERIKQSWSVDVLENGLRSLQLGPVSSSLS | |
| | RSNSR | |
| Felis catus | MEPWRPSPRNPMDRIDPNTFRFHFPNLLYASGRKLCYLCFQ | SEQ ID |
| APOBEC3Ca | VETEDYFSCDDSDRGVFRNKVHPWARCHAEQCFLSWFRDQ | NO: 797 |
| | YPYRDEYYNVTWFLSWSPCPTCAEEVVEFLEEYRNLTLSIFT | |
| | SRLYYFWDPNYQEGLCKLWDAGVQLDIMSCDDFKHCWDN | |
| | FVDHKGMRFQRRNLLKDYDFLAAELQEILR | |
| Human | MALLTAETFRLQFNNKRRLRRPYYPRKALLCYQLTPQNGST | SEQ ID |
| APOBEC3H | PTRGYFENKKKCHAEICFINEIKSMGLDETQCYQVTCYLTW | NO: 798 |
| | SPCSSCAWELVDFIKAHDHLNLGIFASRLYYHWCKPQQKGL | |
| | RLLCGSQVPVEVMGFPEFADCWENFVDHEKPLSFNPYKML | |
| | EELDKNSRAIKRRLERIKIPGVRAQGRYMDILCDAEV | |
| Macaca | MVEPMDPRTFVSNFNNRPILSGLNTVWLCCEVKTKDPSGPP | SEQ ID |
| mulatta | LDAKIFQGKVYSKAKYHPEMRFLRWFHKWRQLHHDQEYK | NO: 799 |
| APOBEC3G | VTWYVSWSPCTRCANSVATFLAKDPKVTLTIFVARLYYFW | |
| | KPDYQQALRILCQKRGGPHATMKIMNYNEFQDCWNKFVD | |
| | GRGKPFKPRNNLPKHYTLLQATLGELLRHLMDPGTFTSNFN | |
| | NKPWVSGQHETYLCYKVERLHNDTWVPLNQHRGFLRNQA | |
| | PNIHGFPKGRHAELCFLDLIPFWKLDGQQYRVTCFTSWSPCF | |
| | SCAQEMAKFISNNEHVSLCIFAARIYDDQGRYQEGLRALHR | |
| | DGAKIAMMNYSEFEYCWDTFVDRQGRPFQPWDGLDEHSQ | |
| | ALSGRLRAI | |
| Pan | MKPHFRNPVERMYQDTFSDNFYNRPILSHRNTVWLCYEVK | SEQ ID |
| troglodytes | TKGPSRPPLDAKIFRGQVYSKLKYHPEMRFFHWFSKWRKL | NO: 800 |
| APOBEC3G | HRDQEYEVTWYISWSPCTKCTRDVATFLAEDPKVTLTIFVA | |
| | RLYYFWDPDYQEALRSLCQKRDGPRATMKIMNYDEFQHC | |
| | WSKFVYSQRELFEPWNNLPKYYILLHIMLGEILRHSMDPPTF | |
| | TSNFNNELWVRGRHETYLCYEVERLHNDTWVLLNQRRGFL | |
| | CNQAPHKHGFLEGRHAELCFLDVIPFWKLDLHQDYRVTCFT | |
| | SWSPCFSCAQEMAKFISNNKHVSLCIFAARIYDDQGRCQEG | |
| | LRTLAKAGAKISIMTYSEFKHCWDTFVDHQGCPFQPWDGLE | |
| | EHSQALSGRLRAILQNQGN | |
| Chlorocebus | MNPQIRNMVEQMEPDIFVYYFNNRPILSGRNTVWLCYEVKT | SEQ ID |
| aethiops | KDPSGPPLDANIFQGKLYPEAKDHPEMKFLHWFRKWRQLH | NO: 801 |
| APOBEC3G | RDQEYEVTWYVSWSPCTRCANSVATFLAEDPKVTLTIFVAR | |
| | LYYFWKPDYQQALRILCQERGGPHATMKIMNYNEFQHCW | |
| | NEFVDGQGKPFKPRKNLPKHYTLLHATLGELLRHVMDPGT | |
| | FTSNFNNKPWVSGQRETYLCYKVERSHNDTWVLLNQHRGF | |
| | LRNQAPDRHGFPKGRHAELCFLDLIPFWKLDDQQYRVTCFT | |
| | SWSPCFSCAQKMAKFISNNKHVSLCIFAARIYDDQGRCQEG | |
| | LRTLHRDGAKIAVMNYSEFEYCWDTFVDRQGRPFQPWDGL | |
| | DEHSQALSGRLRAI | |
| Human | MKPHFRNTVERMYRDTFSYNFYNRPILSRRNTVWLCYEVK | SEQ ID |
| APOBEC3F | TKGPSRPRLDAKIFRGQVYSQPEHHAEMCFLSWFCGNQLPA | NO: 802 |
| | YKCFQITWFVSWTPCPDCVAKLAEFLAEHPNVTLTISAARL | |
| | YYYWERDYRRALCRLSQAGARVKIMDDEEFAYCWENFVY | |
| | SEGQPFMPWYKFDDNYAFLHRTLKEILRNPMEAMYPHIFYF | |
| | HFKNLRKAYGRNESWLCFTMEVVKHHSPVSWKRGVFRNQ | |
| | VDPETHCHAERCFLSWFCDDILSPNTNYEVTWYTSWSPCPE | |
| | CAGEVAEFLARHSNVNLTIFTARLYYFWDTDYQEGLRSLSQ | |
| | EGASVEIMGYKDFKYCWENFVYNDDEPFKPWKGLKYNFLF | |
| | LDSKLQEILE | |
| Human | MKPHFRNTVERMYRDTFSYNFYNRPILSRRNTVWLCYEVK | SEQ ID |
| APOBEC3G | TKGPSRPPLDAKIFRGQVYSELKYHPEMRFFHWFSKWRKLH | NO: 803 |
| | RDQEYEVTWYISWSPCTKCTRDMATFLAEDPKVTLTIFVAR | |
| | LYYFWDPDYQEALRSLCQKRDGPRATMKIMNYDEFQHCW | |
| | SKFVYSQRELFEPWNNLPKYYILLHIMLGEILRHSMDPPTFT | |
| | FNFNNEPWVRGRHETYLCYEVERMHNDTWVLLNQRRGFL | |
| | CNQAPHKHGFLEGRHAELCFLDVIPFWKLDLDQDYRVTCFT | |
| | SWSPCFSCAQEMAKFISKNKHVSLCIFTARIYDDQGRCQEGL | |
| | RTLAEAGAKISIMTYSEFKHCWDTFVDHQGCPFQPWDGLDE | |
| | HSQDLSGRLRAILQNQEN | |
| Human | MNPQIRNPMKAMYPGTFYFQFKNLWEANDRNETWLCFTVE | SEQ ID |
| APOBEC3C | GIKRRSVVSWKTGVFRNQVDSETHCHAERCFLSWFCDDILS | NO: 804 |
| | PNTKYQVTWYTSWSPCPDCAGEVAEFLARHSNVNLTIFTAR | |
| | LYYFQYPCYQEGLRSLSQEGVAVEIMDYEDFKYCWENFVY | |
| | NDNEPFKPWKGLKTNFRLLKRRLRESLQ | |
| Human | MNPQIRNPMERMYRDTFYDNFENEPILYGRSYTWLCYEVKI | SEQ ID |
| APOBEC3B | KRGRSNLLWDTGVFRGQVYFKPQYHAEMCFLSWFCGNQL | NO: 805 |
| | PAYKCFQITWFVSWTPCPDCVAKLAEFLSEHPNVTLTISAAR | |
| | LYYYWERDYRRALCRLSQAGARVTIMDYEEFAYCWENFV | |
| | YNEGQQFMPWYKFDENYAFLHRTLKEILRYLMDPDTFTFNF | |
| | NNDPLVLRRRQTYLCYEVERLDNGTWVLMDQHMGFLCNE | |
| | AKNLLCGFYGRHAELRFLDLVPSLQLDPAQIYRVTWFISWS | |
| | PCFSWGCAGEVRAFLQENTHVRLRIFAARIYDYDPLYKEAL | |
| | QMLRDAGAQVSIMTYDEFEYCWDTFVYRQGCPFQPWDGL | |
| | EEHSQALSGRLRAILQNQGN | |
| Macaca | MALLTAKTFSLQFNNKRRVNKPYYPRKALLCYQLTPQNGS | SEQ ID |
| mulatta | TPTRGHLKNKKKDHAEIRFINKIKSMGLDETQCYQVTCYLT | NO: 806 |
| APOBEC3H | WSPCPSCAGELVDFIKAHRHLNLRIFASRLYYHWRPNYQEG | |
| | LLLLCGSQVPVEVMGLPEFTDCWENFVDHKEPPSFNPSEKL | |
| | EELDKNSQAIKRRLERIKSRSVDVLENGLRSLQLGPVTPSSSI | |
| | RNSR | |
| Human | MNPQIRNPMERMYRDTFYDNFENEPILYGRSYTWLCYEVKI | SEQ ID |
| APOBEC3D | KRGRSNLLWDTGVFRGPVLPKRQSNHRQEVYFRFENHAEM | NO: 807 |
| | CFLSWFCGNRLPANRRFQITWFVSWNPCLPCVVKVTKFLAE | |
| | HPNVTLTISAARLYYYRDRDWRWVLLRLHKAGARVKIMD | |
| | YEDFAYCWENFVCNEGQPFMPWYKFDDNYASLHRTLKEIL | |
| | RNPMEAMYPHIFYFHFKNLLKACGRNESWLCFTMEVTKHH | |
| | SAVFRKRGVFRNQVDPETHCHAERCFLSWFCDDILSPNTNY | |
| | EVTWYTSWSPCPECAGEVAEFLARHSNVNLTIFTARLCYFW | |
| | DTDYQEGLCSLSQEGASVKIMGYKDFVSCWKNFVYSDDEP | |
| | FKPWKGLQTNFRLLKRRLREILQ | |
| Mus | MQPQRLGPRAGMGPFCLGCSHRKCYSPIRNLISQETFKFHFK | SEQ ID |
| musculus | NLGYAKGRKDTFLCYEVTRKDCDSPVSLHHGVFKNKDNIH | NO: 808 |
| APOBEC3 | AEICFLYWFHDKVLKVLSPREEFKITWYMSWSPCFECAEQI | |
| | VRFLATHHNLSLDIFSSRLYNVQDPETQQNLCRLVQEGAQV | |
| | AAMDLYEFKKCWKKFVDNGGRRFRPWKRLLTNFRYQDSK | |
| | LQEILRPCYISVPSSSSSTLSNICLTKGLPETRFWVEGRRMDP | |
| | LSEEEFYSQFYNQRVKHLCYYHRMKPYLCYQLEQFNGQAP | |
| | LKGCLLSEKGKQHAEILFLDKIRSMELSQVTITCYLTWSPCP | |
| | NCAWQLAAFKRDRPDLILHIYTSRLYFHWKRPFQKGLCSLW | |
| | QSGILVDVMDLPQFTDCWTNFVNPKRPFWPWKGLEIISRRT | |
| | QRRLRRIKESWGLQDLVNDFGNLQLGPPMS | |
| Rattus | MSSETGPVAVDPTLRRRIEPHEFEVFFDPRELRKETCLLYEIN | SEQ ID |
| norvegicus | WGGRHSIWRHTSQNTNKHVEVNFIEKFTTERYFCPNTRCSIT | NO: 809 |
| APOBEC1 | WFLSWSPCGECSRAITEFLSRYPHVTLFIYIARLYHHADPRN | |
| | RQGLRDLISSGVTIQIMTEQESGYCWRNFVNYSPSNEAHWP | |
| | RYPHLWVRLYVLELYCIILGLPPCLNILRRKQPQLTFFTIALQ | |
| | SCHYQRLPPHILWATGLK | |
| Human | MTSEKGPSTGDPTLRRRIEPWEFDVFYDPRELRKEACLLYEI | SEQ ID |
| APOBEC1 | KWGMSRKIWRSSGKNTTNHVEVNFIKKFTSERDFHPSMSCS | NO: 810 |
| | ITWFLSWSPCWECSQAIREFLSRHPGVTLVIYVARLFWHMD | |
| | QQNRQGLRDLVNSGVTIQIMRASEYYHCWRNFVNYPPGDE | |
| | AHWPQYPPLWMMLYALELHCIILSLPPCLKISRRWQNHLTF | |
| | FRLHLQNCHYQTIPPHILLATGLIHPSVAWR | |
| Oryctolagus | MASEKGPSNKDYTLRRRIEPWEFEVFFDPQELRKEACLLYEI | SEQ ID |
| cuniculus | KWGASSKTWRSSGKNTTNHVEVNFLEKLTSEGRLGPSTCCS | NO: 811 |
| APOBEC1 | ITWFLSWSPCWECSMAIREFLSQHPGVTLIIFVARLFQHMDR | |
| | RNRQGLKDLVTSGVTVRVMSVSEYCYCWENFVNYPPGKA | |
| | AQWPRYPPRWMLMYALELYCIILGLPPCLKISRRHQKQLTF | |
| | FSLTPQYCHYKMIPPYILLATGLLQPSVPWR | |
| Mus | MSSETGPVAVDPTLRRRIEPHEFEVFFDPRELRKETCLLYEIN | SEQ ID |
| musculus | WGGRHSVWRHTSQNTSNHVEVNFLEKFTTERYFRPNTRCSI | NO: 812 |
| APOBEC1 | TWFLSWSPCGECSRAITEFLSRHPYVTLFIYIARLYHHTDQR | |
| | NRQGLRDLISSGVTIQIMTEQEYCYCWRNFVNYPPSNEAYW | |
| | PRYPHLWVKLYVLELYCIILGLPPCLKILRRKQPQLTFFTITL | |
| | QTCHYQRIPPHLLWATGLK | |
| Mesocricetus | MSSETGPVVVDPTLRRRIEPHEFDAFFDQGELRKETCLLYEI | SEQ ID |
| auratus | RWGGRHNIWRHTGQNTSRHVEINFIEKFTSERYFYPSTRCSI | NO: 813 |
| APOBEC1 | VWFLSWSPCGECSKAITEFLSGHPNVTLFIYAARLYHHTDQ | |
| | RNRQGLRDLISRGVTIRIMTEQEYCYCWRNFVNYPPSNEVY | |
| | WPRYPNLWMRLYALELYCIHLGLPPCLKIKRRHQYPLTFFR | |
| | LNLQSCHYQRIPPHILWATGFI | |
| Monodelphis | MNSKTGPSVGDATLRRRIKPWEFVAFFNPQELRKETCLLYEI | SEQ ID |
| domestica | KWGNQNIWRHSNQNTSQHAEINFMEKFTAERHFNSSVRCSI | NO: 814 |
| APOBEC1 | TWFLSWSPCWECSKAIRKFLDHYPNVTLAIFISRLYWHMDQ | |
| | QHRQGLKELVhgVTIQIMSYSEYHYCWRNFVDYPQGEEDY | |
| | WPKYPYLWIMLYVLELHCIILGLPPCLKISGSHSNQLALFSL | |
| | DLQDCHYQKIPYNVLVATGLVQPFVTWR | |
| Pongo | MTSEKGPSTGDPTLRRRIESWEFDVFYDPRELRKETCLLYEI | SEQ ID |
| pygmaeus | KWGMSRKIWRSSGKNTTNHVEVNFIKKFTSERRFHSSISCSI | NO: 815 |
| APOBEC1 | TWFLSWSPCWECSQAIREFLSQHPGVTLVIYVARLFWHMD | |
| | QRNRQGLRDLVNSGVTIQIMRASEYYHCWRNFVNYPPGDE | |
| | AHWPQYPPLWMMLYALELHCIILSLPPCLKISRRWQNHLAF | |
| | FRLHLQNCHYQTIPPHILLATGLIHPSVTWR | |
| Bos taurus | MAQKEEAAAAAEPASQNGEEVENLEDPEKLKELIELPPFEIV | SEQ ID |
| APOBEC2 | TGERLPAHYFKFQFRNVEYSSGRNKTFLCYVVEAQSKGGQ | NO: 816 |
| | VQASRGYLEDEHATNHAEEAFFNSIMPTFDPALRYMVTWY | |
| | VSSSPCAACADRIVKTLNKTKNLRLLILVGRLFMWEEPEIQA | |
| | ALRKLKEAGCRLRIMKPQDFEYIWQNFVEQEEGESKAFEPW | |
| | EDIQENFLYYEEKLADILK | |
| Mus | MAQKEEAAEAAAPASQNGDDLENLEDPEKLKELIDLPPFEI | SEQ ID |
| musculus | VTGVRLPVNFFKFQFRNVEYSSGRNKTFLCYVVEVQSKGG | NO: 817 |
| APOBEC2 | QAQATQGYLEDEHAGAHAEEAFFNTILPAFDPALKYNVTW | |
| | YVSSSPCAACADRILKTLSKTKNLRLLILVSRLFMWEEPEVQ | |
| | AALKKLKEAGCKLRIMKPQDFEYIWQNFVEQEEGESKAFEP | |
| | WEDIQENFLYYEEKLADILK | |
| Human | MAQKEEAAVATEAASQNGEDLENLDDPEKLKELIELPPFEIV | SEQ ID |
| APOBEC2 | TGERLPANFFKFQFRNVEYSSGRNKTFLCYVVEAQGKGGQ | NO: 818 |
| | VQASRGYLEDEHAAAHAEEAFFNTILPAFDPALRYNVTWY | |
| | VSSSPCAACADRIIKTLSKTKNLRLLILVGRLFMWEEPEIQAA | |
| | LKKLKEAGCKLRIMKPQDFEYVWQNFVEQEEGESKAFQPW | |
| | EDIQENFLYYEEKLADILK | |
| Pongo | MAQKEEAAAATEAASQNGEDLENLDDPEKLKELIELPPFEIV | SEQ ID |
| pygmaeus | TGERLPANFFKFQFRNVEYSSGRNKTFLCYVVEAQGKGGQ | NO: 819 |
| APOBEC2 | VQASRGYLEDEHAAAHAEEAFFNTILPAFDPALRYNVTWY | |
| | VSSSPCAACADRIIKTLSKTKNLRLLILVGRLFMWEELEIQD | |
| | ALKKLKEAGCKLRIMKPQDFEYVWQNFVEQEEGESKAFQP | |
| | WEDIQENFLYYEEKLADILK | |
| Human | MEPIYEEYLANHGTIVKPYYWLSFSLDCSNCPYHIRTGEEAR | SEQ ID |
| APOBEC4 | VSLTEFCQIFGFPYGTTFPQTKHLTFYELKTSSGSLVQKGHA | NO: 820 |
| | SSCTGNYIHPESMLFEMNGYLDSAIYNNDSIRHIILYSNNSPC | |
| | NEANHCCISKMYNFLITYPGITLSIYFSQLYHTEMDFPASAW | |
| | NREALRSLASLWPRVVLSPISGGIWHSVLHSFISGVSGSHVF | |
| | QPILTGRALADRHNAYEINAITGVKPYFTDVLLQTKRNPNTK | |
| | AQEALESYPLNNAFPGQFFQMPSGQLQPNLPPDLRAPVVFV | |
| | LVPLRDLPPMHMGQNPNKPRNIVRHLNMPQMSFQETKDLG | |
| | RLPTGRSVEIVEITEQFASSKEADEKKKKKGKK | |
| Rattus | MEPLYEEYLThgTIVKPYYWLSVSLNCTNCPYHIRTGEEARV | SEQ ID |
| norvegicus | PYTEFHQTFGFPWSTYPQTKHLTFYELRSSSGNLIQKGLASN | NO: 821 |
| APOBEC4 | CTGSHTHPESMLFERDGYLDSLIFHDSNIRHIILYSNNSPCDE | |
| | ANHCCISKMYNFLMNYPEVTLSVFFSQLYHTENQFPTSAWN | |
| | REALRGLASLWPQVTLSAISGGIWQSILETFVSGISEGLTAVR | |
| | PFTAGRTLTDRYNAYEINCITEVKPYFTDALHSWQKENQDQ | |
| | KVWAASENQPLHNTTPAQWQPDMSQDCRTPAVFMLVPYR | |
| | DLPPIHVNPSPQKPRTVVRHLNTLQLSASKVKALRKSPSGRP | |
| | VKKEEARKGSTRSQEANETNKSKWKKQTLFIKSNICHLLER | |
| | EQKKIGILSSWSV | |
| Mus | MEPLYEEILTQGGTIVKPYYWLSLSLGCTNCPYHIRTGEEAR | SEQ ID |
| musculus | VPYTEFHQTFGFPWSTYPQTKHLTFYELRSSSKNLIQKGLAS | NO: 822 |
| APOBEC4 | NCTGSHNHPEAMLFEKNGYLDAVIFHNSNIRHIILYSNNSPC | |
| | NEAKHCCISKMYNFLMNYPEVTLSVFFSQLYHTEKQFPTSA | |
| | WNRKALQSLASLWPQVTLSPICGGLWHAILEKFVSNISGSTV | |
| | PQPFIAGRILADRYNTYEINSIIAAKPYFTDGLLSRQKENQNR | |
| | EAWAAFEKHPLGSAAPAQRQPTRGQDPRTPAVLMLVSNRD | |
| | LPPIHVGSTPQKPRTVVRHLNMLQLSSFKVKDVKKPPSGRP | |
| | VEEVEVMKESARSQKANKKNRSQWKKQTLVIKPRICRLLER | |
| Macaca | MEPTYEEYLANHGTIVKPYYWLSFSLDCSNCPYHIRTGEEA | SEQ ID |
| fascicularis | RVSLTEFCQIFGFPYGTTYPQTKHLTFYELKTSSGSLVQKGH | NO: 823 |
| ADAT1 | ASSCTGNYIHPESMLFEMNGYLDSAIYNNDSIRHIILYCNNSP | |
| | CNEANHCCISKVYNFLITYPGITLSIYFSQLYHTEMDFPASA | |
| | WNREALRSLASLWPRVVLSPISGGIWHSVLHSFVSGVSGSH | |
| | VFQPILTGRALTDRYNAYEINAITGVKPFFTDVLLHTKRNPN | |
| | TKAQMALESYPLNNAFPGQSFQMTSGIPPDLRAPVVFVLLP | |
| | LRDLPPMHMGQDPNKPRNIIRHLNMPQMSFQETKDLERLPT | |
| | RRSVETVEITERFASSKQAEEKTKKKKGKK | |
| Human | MDSLLMNRRKFLYQFKNVRWAKGRRETYLCYVVKRRDSA | SEQ ID |
| AICDA | TSFSLDFGYLRNKNGCHVELLFLRYISDWDLDPGRCYRVTW | NO: 824 |
| | FTSWSPCYDCARHVADFLRGNPNLSLRIFTARLYFCEDRKA | |
| | EPEGLRRLHRAGVQIAIMTFKDYFYCWNTFVENHERTFKA | |
| | WEGLHENSVRLSRQLRRILLPLYEVDDLRDAFRTLGL | |
| Mus | MDSLLMKQKKFLYHFKNVRWAKGRHETYLCYVVKRRDSA | SEQ ID |
| musculus | TSCSLDFGHLRNKSGCHVELLFLRYISDWDLDPGRCYRVTW | NO: 825 |
| AICDA | FTSWSPCYDCARHVAEFLRWNPNLSLRIFTARLYFCEDRKA | |
| | EPEGLRRLHRAGVQIGIMTFKDYFYCWNTFVENRERTFKA | |
| | WEGLHENSVRLTRQLRRILLPLYEVDDLRDAFRMLGF | |
| Canis lupus | MDSLLMKQRKFLYHFKNVRWAKGRHETYLCYVVKRRDSA | SEQ ID |
| familiaris | TSFSLDFGHLRNKSGCHVELLFLRYISDWDLDPGRCYRVTW | NO: 826 |
| (Dog) AICDA | FTSWSPCYDCARHVADFLRGYPNLSLRIFAARLYFCEDRKA | |
| | EPEGLRRLHRAGVQIAIMTFKDYFYCWNTFVENREKTFKA | |
| | WEGLHENSVRLSRQLRRILLPLYEVDDLRDAFRTLGL | |
| Bos taurus | MDSLLKKQRQFLYQFKNVRWAKGRHETYLCYVVKRRDSP | SEQ ID |
| (Bovine) | TSFSLDFGHLRNKAGCHVELLFLRYISDWDLDPGRCYRVTW | NO: 827 |
| AICDA | FTSWSPCYDCARHVADFLRGYPNLSLRIFTARLYFCDKERK | |
| | AEPEGLRRLHRAGVQIAIMTFKDYFYCWNTFVENHERTFKA | |
| | WEGLHENSVRLSRQLRRILLPLYEVDDLRDAFRTLGL | |

In some embodiments, the nucleotide deaminase is an adenosine deaminase. In some embodiments, the adenosine deaminase comprises a sequence of SEQ ID NOs: 828-920.

In some embodiments, the first fusion protein further comprises an uracil glycosylase inhibitor (UGI).

The “Uracil Glycosylase Inhibitor” (UGI), which can be prepared from Bacillus subtilis bacteriophage PBS1, is a small protein (9.5 kDa) which inhibits E. coli uracil-DNA glycosylase (UDG) as well as UDG from other species. Inhibition of UDG occurs by reversible protein binding with a 1:1 UDG:UGI stoichiometry. UGI is capable of dissociating UDG-DNA complexes. A non-limiting example of UGI is found in Bacillus phage AR9 (YP_009283008.1). In some embodiments, the UGI comprises the amino acid sequence of SEQ ID NO: 145 or has at least 70%, 75%, 80%, 85%, 90% or 95% sequence identity to SEQ ID NO: 145 and retains the uracil glycosylase inhibition activity.

In some embodiments, the first fusion protein further comprises a nuclear localization sequence (NLS).

A “nuclear localization signal or sequence” (NLS) is an amino acid sequence that tags a protein for import into the cell nucleus by nuclear transport. Typically, this signal consists of one or more short sequences of positively charged lysines or arginines exposed on the protein surface. Different nuclear localized proteins may share the same NLS. A non-limiting example of NLS is the internal SV40 nuclear localization sequence (iNLS).

In some embodiments, a peptide linker is optionally provided between each of the fragments in any of the fusion proteins. In some embodiments, the peptide linker has from 1 to 100 amino acid residues (or 3-20, 4-15, without limitation). In some embodiments, at least 10%, 20%, 30⁰/a, 40%, 50%, 60%, 70%, 80% or 90% of the amino acid residues of peptide linker are amino acid residues selected from the group consisting of alanine, glycine, cysteine, and serine.

The term “Cas protein” or “clustered regularly interspaced short palindromic repeats (CRISPR)-associated (Cas) protein” refers to RNA-guided DNA endonuclease enzymes associated with the CRISPR (Clustered Regularly Interspaced Short Palindromic Repeats) adaptive immunity system in Streptococcus pyogenes, as well as other bacteria. Cas proteins include Cas9 proteins, Cas12a (Cpf1) proteins, Cas12b (formerly known as C2c1) proteins, Cas13 proteins and various engineered counterparts. Example Cas proteins include SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RITA FnCas9, KKH SaCas9, NmeCas9, StCas9, CjCas9, AsCpf1, FnCpf1, SsCpf1, PcCpf1 BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, RanCas13b and those provided in Table 10 below.

TABLE 10
Exemplary Cas Proteins

| Cas protein types | Cas proteins |
|---|---|
| Cas9 proteins | Cas9 from Staphylococcus aureus (SaCas9) |
| | Cas9 from Neisseria meningitidis (NmeCas9) |
| | Cas9 from Streptococcus thermophilus (StCas9) |
| | Cas9 from Campylobacter jejuni (CjCas9) |
| Cas12a (Cpf1) | Cas12a (Cpf1) from Acidaminococcus sp BV3L6 (AsCpf1) |
| proteins | Cas12a (Cpf1) from Francisella novicida sp BV3L6 (FnCpf1) |
| | Cas12a (Cpf1) from Smithella sp SC K08D17 (SsCpf1) |
| | Cas12a (Cpf1) from Porphyromonas crevioricanis (PcCpf1) |
| | Cas12a (Cpf1) from Butyrivibrio proteoclasticus (BpCpf1) |
| | Cas12a (Cpf1) from Candidatus Methanoplasma termitum |
| | (CmtCpf1) |
| | Cas12a (Cpf1) from Leptospira inadai (LiCpf1) |
| | Cas12a (Cpf1) from Porphyromonas macacae (PmCpf1) |
| | Cas12a (Cpf1) from Peregrinibacteria bacterium GW2011 WA2 |
| | 33 10 (Pb3310Cpf1) |
| | Cas12a (Cpf1) from Parcubacteria bacterium GW2011 GWC2 44 |
| | 17 (Pb4417Cpf1) |
| | Cas12a (Cpf1) from Butyrivibrio sp. NC3005 (BsCpf1) |
| | Cas12a (Cpf1) from Eubacterium eligens (EeCpf1) |
| Cas12b (C2c1) | Cas12b (C2c1) Bacillus hisashii (BhCas12b) |
| proteins | Cas12b (C2c1) Bacillus hisashii with a gain-of-function |
| | mutation (see, e.g., Strecker et al., Nature Communications 10 (article |
| | 212) (2019) |
| | Cas12b (C2c1) Alicyclobacillus kakegawensis (AkCas12b) |
| | Cas12b (C2c1) Elusimicrobia bacterium (EbCas12b) |
| | Cas12b (C2c1) Laceyella sediminis (Ls) (LsCas12b) |
| Cas13 proteins | Cas13d from Ruminococcus flavefaciens XPD3002 (RfCas13d) |
| | Cas13a from Leptotrichia wadei (LwaCas13a) |
| | Cas13b from Prevotella sp. P5-125 (PspCas13b) |
| | Cas13b from Porphyromonas gulae (PguCas13b) |
| | Cas13b from Riemerella anatipestifer (RanCas13b) |
| Engineered Cas | Nickases (mutation in one nuclease domain) |
| proteins | Catalytically inactive mutant (dCas9; mutations in both of the |
| | nuclease domains) |
| | Enhanced variants with improved specificity (see, e.g., |
| | Chen et al., Nature, 550, 407-410 (2017) |

In some embodiments, the Cas protein comprise an amino acid sequence of any one of SEQ ID NOs: 733-784. (Table 25)

TABLE 25

| | | SEQ |
|---|---|---|
| Name | Sequence | ID |
| SpCas9 | MDKKYSIGLDIGTNSVGWAVITDEYKVPSKKFKVLGNTDRHSIKKNL | SEQ |
| | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 733 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKRYTSTKEVLDATLIHQSITGLYETRIDLSQLGGD | |
| dSpCas9 | MDKKYSIGLAIGTNSVGWAVITDEYKVPSKKFKVLGNTDRHSIKKNL | SEQ |
| | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 734 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDAIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKRYTSTKEVLDATLIHQSITGLYETRIDLSQLGGD | |
| nSpCas9 | MDKKYSIGLAIGTNSVGWAVITDEYKVPSKKFKVLGNTDRHSIKKNL | SEQ |
| (D10A) | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 735 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKRYTSTKEVLDATLIHQSITGLYETRIDLSQLGGD | |
| nSpCas9 | MDKKYSIGLDIGTNSVGWAVITDEYKVPSKKFKVLGNTDRHSIKKNL | SEQ |
| (H840A) | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 736 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDAIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKRYTSTKEVLDATLIHQSITGLYETRIDLSQLGGD | |
| FnCas9 | MNFKILPIAIDLGVKNTGVFSAFYQKGTSLERLDNKNGKVYELSKDS | SEQ |
| | YTLLMNNRTARRHQRRGIDRKQLVKRLFKLIWTEQLNLEWDKDTQ | ID |
| | QAISFLFNRRGFSFITDGYSPEYLNIVPEQVKAILMDIFDDYNGEDDLD | NO: |
| | SYLKLATEQESKISEIYNKLMQKILEFKLMKLCTDIKDDKVSTKTLKE | 737 |
| | ITSYEFELLADYLANYSESLKTQKFSYTDKQGNLKELSYYHHDKYNI | |
| | QEFLKRHATINDRILDTLLTDDLDIWNFNFEKFDFDKNEEKLQNQED | |
| | KDHIQAHLHHFVFAVNKIKSEMASGGRHRSQYFQEITNVLDENNHQ | |
| | EGYLKNFCENLHNKKYSNLSVKNLVNLIGNLSNLELKPLRKYFNDKI | |
| | HAKADHWDEQKFTETYCHWILGEWRVGVKDQDKKDGAKYSYKDL | |
| | CNELKQKVTKAGLVDFLLELDPCRTIPPYLDNNNRKPPKCQSLILNPK | |
| | FLDNQYPNWQQYLQELKKLQSIQNYLDSFETDLKVLKSSKDQPYFV | |
| | EYKSSNQQIASGQRDYKDLDARILQFIFDRVKASDELLLNEIYFQAKK | |
| | LKQKASSELEKLESSKKLDEVIANSQLSQILKSQHTNGIFEQGTFLHL | |
| | VCKYYKQRQRARDSRLYIMPEYRYDKKLHKYNNTGRFDDDNQLLT | |
| | YCNHKPRQKRYQLLNDLAGVLQVSPNFLKDKIGSDDDLFISKWLVE | |
| | HIRGFKKACEDSLKIQKDNRGLLNHKINIARNTKGKCEKEIFNLICKIE | |
| | GSEDKKGNYKHGLAYELGVLLFGEPNEASKPEFDRKIKKFNSIYSFA | |
| | QIQQIAFAERKGNANTCAVCSADNAHRMQQIKITEPVEDNKDKIILSA | |
| | KAQRLPAIPTRIVDGAVKKMATILAKNIVDDNWQNIKQVLSAKHQL | |
| | HIPIITESNAFEFEPALADVKGKSLKDRRKKALERISPENIFKDKNNRI | |
| | KEFAKGISAYSGANLTDGDFDGAKEELDHIIPRSHKKYGTLNDEANLI | |
| | CVTRGDNKNKGNRIFCLRDLADNYKLKQFETTDDLEIEKKIADTIWD | |
| | ANKKDFKFGNYRSFINLTPQEQKAFRHALFLADENPIKQAVIRAINNR | |
| | NRTFVNGTQRYFAEVLANNIYLRAKKENLNTDKISFDYFGIPTIGNGR | |
| | GIAEIRQLYEKVDSDIQAYAKGDKPQASYSHLIDAMLAFCIAADEHR | |
| | NDGSIGLEIDKNYSLYPLDKNTGEVFTKDIFSQIKITDNEFSDKKLVRK | |
| | KAIEGFNTHRQMTRDGIYAENYLPILIHKELNEVRKGYTWKNSEEIKI | |
| | FKGKKYDIQQLNNLVYCLKFVDKPISIDIQISTLEELRNILTTNNIAAT | |
| | AEYYYINLKTQKLHEYYIENYNTALGYKKYSKEMEFLRSLAYRSER | |
| | VKIKSIDDVKQVLDKDSNFIIGKITLPFKKEWQRLYREWQNTTIKDDY | |
| | EFLKSFFNVKSITKLHKKVRKDFSLPISTNEGKFLVKRKTWDNNFIYQ | |
| | ILNDSDSRADGTKPFIPAFDISKNEIVEAIIDSFTSKNIFWLPKNIELQK | |
| | VDNKNIFAIDTSKWFEVETPSDLRDIGIATIQYKIDNNSRPKVRVKLD | |
| | YVIDDDSKINYFMNHSLLKSRYPDKVLEILKQSTIIEFESSGFNKTIKE | |
| | MLGMKLAGIYNETSNN | |
| St1Cas9 | MGSDLVLGLDIGIGSVGVGILNKVTGEIIHKNSRIFPAAQAENNLVRR | SEQ |
| | TNRQGRRLARRKKHRRVRLNRLFEESGLITDFTKISINLNPYQLRVKG | ID |
| | LTDELSNEELFIALKNMVKHRGISYLDDASDDGNSSVGDYAQIVKEN | NO: |
| | SKQLETKTPGQIQLERYQTYGQLRGDFTVEKDGKKHRLINVFPTSAY | 738 |
| | RSEALRILQTQQEFNPQITDEFINRYLEILTGKRKYYHGPGNEKSRTD | |
| | YGRYRTSGETLDNIFGILIGKCTFYPDEFRAAKASYTAQEFNLLNDLN | |
| | NLTVPTETKKLSKEQKNQIINYVKNEKAMGPAKLFKYIAKLLSCDVA | |
| | DIKGYRIDKSGKAEIHTFEAYRKMKTLETLDIEQMDRETLDKLAYVL | |
| | TLNTEREGIQEALEHEFADGSFSQKQVDELVQFRKANSSIFGKGWHN | |
| | FSVKLMMELIPELYETSEEQMTILTRLGKQKTTSSSNKTKYIDEKLLT | |
| | EEIYNPVVAKSVRQAIKIVNAAIKEYGDFDNIVIEMARETNEDDEKK | |
| | AIQKIQKANKDEKDAAMLKAANQYNGKAELPHSVFHGHKQLATKI | |
| | RLWHQQGERCLYTGKTISIHDLINNSNQFEVDHILPLSITFDDSLANK | |
| | VLVYATANQEKGQRTPYQALDSMDDAWSFRELKAFVRESKTLSNK | |
| | KKEYLLTEEDISKFDVRKKFIERNLVDTRYASRVVLNALQEHFRAHK | |
| | IDTKVSVVRGQFTSQLRRHWGIEKTRDTYHHHAVDALIIAASSQLNL | |
| | WKKQKNTLVSYSEDQLLDIETGELISDDEYKESVFKAPYQHFVDTLK | |
| | SKEFEDSILFSYQVDSKFNRKISDATIYATRQAKVGKDKADETYVLG | |
| | KIKDIYTQDGYDAFMKIYKKDKSKFLMYRHDPQTFEKVIEPILENYP | |
| | NKQINEKGKEVPCNPFLKYKEEHGYIRKYSKKGNGPEIKSLKYYDSK | |
| | LGNHIDITPKDSNNKVVLQSVSPWRADVYFNKTTGKYEILGLKYADL | |
| | QFEKGTGTYKISQEKYNDIKKKEGVDSDSEFKFTLYKNDLLLVKDTE | |
| | TKEQQLFRFLSRTMPKQKHYVELKPYDKQKFEGGEALIKVLGNVAN | |
| | SGQCKKGLGKSNISIYKVRTDVLGNQHIIKNEGDKPKLDF | |
| St3Cas9 | MTKPYSIGLDIGTNSVGWAVTTDNYKVPSKKMKVLGNTSKKYIKKN | SEQ |
| | LLGVLLFDSGITAEGRRLKRTARRRYTRRRNRILYLQEIFSTEMATLD | ID |
| | DAFFQRLDDSFLVPDDKRDSKYPIFGNLVEEKAYHDEFPTIYHLRKY | NO: |
| | LADSTKKADLRLVYLALAHMIKYRGHFLIEGEFNSKNNDIQKNFQDF | 739 |
| | LDTYNAIFESDLSLENSKQLEEIVKDKISKLEKKDRILKLFPGEKNSGI | |
| | FSEFLKLIVGNQADFRKCFNLDEKASLHFSKESYDEDLETLLGYIGDD | |
| | YSDVFLKAKKLYDAILLSGFLTVTDNETEAPLSSAMIKRYNEHKEDL | |
| | ALLKEYIRNISLKTYNEVFKDDTKNGYAGYIDGKTNQEDFYVYLKK | |
| | LLAEFEGADYFLEKIDREDFLRKQRTFDNGSIPYQIHLQEMRAILDKQ | |
| | AKFYPFLAKNKERIEKILTFRIPYYVGPLARGNSDFAWSIRKRNEKITP | |
| | WNFEDVIDKESSAEAFINRMTSFDLYLPEEKVLPKHSLLYETFNVYN | |
| | ELTKVRFIAESMRDYQFLDSKQKKDIVRLYFKDKRKVTDKDIIEYLH | |
| | AIYGYDGIELKGIEKQFNSSLSTYHDLLNIINDKEFLDDSSNEAIIEEIIH | |
| | TLTIFEDREMIKQRLSKFENIFDKSVLKKLSRRHYTGWGKLSAKLING | |
| | IRDEKSGNTILDYLIDDGISNRNFMQLIHDDALSFKKKIQKAQIIGDED | |
| | KGNIKEVVKSLPGSPAIKKGILQSIKIVDELVKVMGGRKPESIVVEMA | |
| | RENQYTNQGKSNSQQRLKRLEKSLKELGSKILKENIPAKLSKIDNNA | |
| | LQNDRLYLYYLQNGKDMYTGDDLDIDRLSNYDIDHIIPQAFLKDNSI | |
| | DNKVLVSSASNRGKSDDVPSLEVVKKRKTFWYQLLKSKLISQRKFD | |
| | NLTKAERGGLSPEDKAGFIQRQLVETRQITKHVARLLDEKENNKKDE | |
| | NNRAVRTVKIITLKSTLVSQFRKDFELYKVREINDFHHAHDAYLNAV | |
| | VASALLKKYPKLEPEFVYGDYPKYNSFRERKSATEKVYFYSNIMNIF | |
| | KKSISLADGRVIERPLIEVNEETGESVWNKESDLATVRRVLSYPQVN | |
| | VVKKVEEQNHGLDRGKPKGLFNANLSSKPKPNSNENLVGAKEYLDP | |
| | KKYGGYAGISNSFTVLVKGTIEKGAKKKITNVLEFQGISILDRINYRK | |
| | DKLNFLLEKGYKDIELIIELPKYSLFELSDGSRRMLASILSTNNKRGEI | |
| | HKGNQIFLSQKFVKLLYHAKRISNTINENHRKYVENHKKEFEELFYYI | |
| | LEFNENYVGAKKNGKLLNSAFQSWQNHSIDELCSSFIGPTGSERKGL | |
| | FELTSRGSAADFEFLGVKIPRYRDYTPSSLLKDATLIHQSVTGLYETRI | |
| | DLAKLGEG | |
| NmCas9 | MAAFKPNSINYILGLDIGIASVGWAMVEIDEEENPIRLIDLGVRVFER | SEQ |
| | AEVPKTGDSLAMARRLARSVRRLTRRRAHRLLRTRRLLKREGVLQA | ID |
| | ANFDENGLIKSLPNTPWQLRAAALDRKLTPLEWSAVLLHLIKHRGYL | NO: |
| | SQRKNEGETADKELGALLKGVAGNAHALQTGDFRTPAELALNKFEK | 740 |
| | ESGHIRNQRSDYSHTFSRKDLQAELILLFEKQKEFGNPHVSGGLKEGI | |
| | ETLLMTQRPALSGDAVQKMLGHCTFEPAEPKAAKNTYTAERFIWLT | |
| | KLNNLRILEQGSERPLTDTERATLMDEPYRKSKLTYAQARKLLGLED | |
| | TAFFKGLRYGKDNAEASTLMEMKAYHAISRALEKEGLKDKKSPLNL | |
| | SPELQDEIGTAFSLFKTDEDITGRLKDRIQPEILEALLKHISFDKFVQIS | |
| | LKALRRIVPLMEQGKRYDEACAEIYGDHYGKKNTEEKIYLPPIPADEI | |
| | RNPVVLRALSQARKVINGVVRRYGSPARIHIETAREVGKSFKDRKEIE | |
| | KRQEENRKDREKAAAKFREYFPNFVGEPKSKDILKLRLYEQQHGKC | |
| | LYSGKEINLGRLNEKGYVEIDHALPFSRTWDDSFNNKVLVLGSENQN | |
| | KGNQTPYEYFNGKDNSREWQEFKARVETSRFPRSKKQRILLQKFDE | |
| | DGFKERNLNDTRYVNRFLCQFVADRMRLTGKGKKRVFASNGQITNL | |
| | LRGFWGLRKVRAENDRHHALDAVVVACSTVAMQQKITRFVRYKE | |
| | MNAFDGKTIDKETGEVLHQKTHFPQPWEFFAQEVMIRVFGKPDGKP | |
| | EFEEADTLEKLRTLLAEKLSSRPEAVHEYVTPLFVSRAPNRKMSGQG | |
| | HMETVKSAKRLDEGVSVLRVPLTQLKLKDLEKMVNREREPKLYEAL | |
| | KARLEAHKDDPAKAFAEPFYKYDKAGNRTQQVKAVRVEQVQKTG | |
| | VWVRNHNGIADNATMVRVDVFEKGDKYYLVPIYSWQVAKGILPDR | |
| | AVVQGKDEEDWQLIDDSFNFKFSLHPNDLVEVITKKARMFGYFASC | |
| | HRGTGNINIRIHDLDHKIGKNGILEGIGVKTALSFQKYQIDELGKEIRP | |
| | CRLKKRPPVR | |
| SaCas9 | MKRNYILGLDIGITSVGYGIIDYETRDVIDAGVRLFKEANVENNEGRR | SEQ |
| | SKRGARRLKRRRRHRIQRVKKLLFDYNLLTDHSELSGINPYEARVKG | ID |
| | LSQKLSEEEFSAALLHLAKRRGVHNVNEVEEDTGNELSTKEQISRNS | NO: |
| | KALEEKYVAELQLERLKKDGEVRGSINRFKTSDYVKEAKQLLKVQK | 741 |
| | AYHQLDQSFIDTYIDLLETRRTYYEGPGEGSPFGWKDIKEWYEMLM | |
| | GHCTYFPEELRSVKYAYNADLYNALNDLNNLVITRDENEKLEYYEK | |
| | FQIIENVFKQKKKPTLKQIAKEILVNEEDIKGYRVTSTGKPEFTNLKV | |
| | YHDIKDITARKEIIENAELLDQIAKILTIYQSSEDIQEELTNLNSELTQE | |
| | EIEQISNLKGYTGTHNLSLKAINLILDELWHTNDNQIAIFNRLKLVPK | |
| | KVDLSQQKEIPTTLVDDFILSPVVKRSFIQSIKVINAIIKKYGLPNDIIIE | |
| | LAREKNSKDAQKMINEMQKRNRQTNERIEEIIRTTGKENAKYLIEKIK | |
| | LHDMQEGKCLYSLEAIPLEDLLNNPFNYEVDHIIPRSVSFDNSFNNKV | |
| | LVKQEENSKKGNRTPFQYLSSSDSKISYETFKKHILNLAKGKGRISKT | |
| | KKEYLLEERDINRFSVQKDFINRNLVDTRYATRGLMNLLRSYFRVNN | |
| | LDVKVKSINGGFTSFLRRKWKFKKERNKGYKHHAEDALIIANADFIF | |
| | KEWKKLDKAKKVMENQMFEEKQAESMPEIETEQEYKEIFITPHQIKH | |
| | IKDFKDYKYSHRVDKKPNRELINDTLYSTRKDDKGNTLIVNNLNGLY | |
| | DKDNDKLKKLINKSPEKLLMYHHDPQTYQKLKLIMEQYGDEKNPLY | |
| | KYYEETGNYLTKYSKKDNGPVIKKIKYYGNKLNAHLDITDDYPNSR | |
| | NKVVKLSLKPYRFDVYLDNGVYKFVTVKNLDVIKKENYYEVNSKC | |
| | YEEAKKLKKISNQAEFIASFYNNDLIKINGELYRVIGVNNDLLNRIEV | |
| | NMIDITYREYLENMNDKRPPRIIKTIASKTQSIKKYSTDILGNLYEVKS | |
| | KKHPQIIKKG | |
| AsCpf1 | MTQFEGFTNLYQVSKTLRFELIPQGKTLKHIQEQGFIEEDKARNDHY | SEQ |
| | KELKPIIDRIYKTYADQCLQLVQLDWENLSAAIDSYRKEKTEETRNA | ID |
| | LIEEQATYRNAIHDYFIGRTDNLTDAINKRHAEIYKGLFKAELFNGKV | NO: |
| | LKQLGTVTTTEHENALLRSFDKFTTYFSGFYENRKNVFSAEDISTAIP | 742 |
| | HRIVQDNFPKFKENCHIFTRLITAVPSLREHFENVKKAIGIFVSTSIEEV | |
| | FSFPFYNQLLTQTQIDLYNQLLGGISREAGTEKIKGLNEVLNLAIQKN | |
| | DETAHIIASLPHRFIPLFKQILSDRNTLSFILEEFKSDEEVIQSFCKYKTL | |
| | LRNENVLETAEALFNELNSIDLTHIFISHKKLETISSALCDHWDTLRN | |
| | ALYERRISELTGKITKSAKEKVQRSLKHEDINLQEIISAAGKELSEAFK | |
| | QKTSEILSHAHAALDQPLPTTLKKQEEKEILKSQLDSLLGLYHLLDWF | |
| | AVDESNEVDPEFSARLTGIKLEMEPSLSFYNKARNYATKKPYSVEKF | |
| | KLNFQMPTLASGWDVNKEKNNGAILFVKNGLYYLGIMPKQKGRYK | |
| | ALSFEPTEKTSEGFDKMYYDYFPDAAKMIPKCSTQLKAVTAHFQTHT | |
| | TPILLSNNFIEPLEITKEIYDLNNPEKEPKKFQTAYAKKTGDQKGYRE | |
| | ALCKWIDFTRDFLSKYTKTTSIDLSSLRPSSQYKDLGEYYAELNPLLY | |
| | HISFQRIAEKEIMDAVETGKLYLFQIYNKDFAKGHHGKPNLHTLYWT | |
| | GLFSPENLAKTSIKLNGQAELFYRPKSRMKRMAHRLGEKMLNKKLK | |
| | DQKTPIPDTLYQELYDYVNHRLSHDLSDEARALLPNVITKEVSHEIIK | |
| | DRRFTSDKFFFHVPITLNYQAANSPSKFNQRVNAYLKEHPETPIIGIDR | |
| | GERNLIYITVIDSTGKILEQRSLNTIQQFDYQKKLDNREKERVAARQA | |
| | WSVVGTIKDLKQGYLSQVIHEIVDLMIHYQAVVVLENLNFGFKSKRT | |
| | GIAEKAVYQQFEKMLIDKLNCLVLKDYPAEKVGGVLNPYQLTDQFT | |
| | SFAKMGTQSGFLFYVPAPYTSKIDPLTGFVDPFVWKTIKNHESRKHF | |
| | LEGFDFLHYDVKTGDFILHFKMNRNLSFQRGLPGFMPAWDIVFEKNE | |
| | TQFDAKGTPFIAGKRIVPVIENHRFTGRYRDLYPANELIALLEEKGIVF | |
| | RDGSNILPKLLENDDSHAIDTMVALIRSVLQMRNSNAATGEDYINSP | |
| | VRDLNGVCFDSRFQNPEWPMDADANGAYHIALKGQLLLNHLKESK | |
| | DLKLQNGISNQDWLAYIQELRN | |
| LbCpf1 | MAASKLEKFTNCYSLSKTLRFKAIPVGKTQENIDNKRLLVEDEKRAE | SEQ |
| | DYKGVKKLLDRYYLSFINDVLHSIKLKNLNNYISLFRKKTRTEKENK | ID |
| | ELENLEINLRKEIAKAFKGAAGYKSLFKKDIIETILPEAADDKDEIALV | NO: |
| | NSFNGFTTAFTGFFDNRENMFSEEAKSTSIAFRCINENLTRYISNMDIF | 743 |
| | EKVDAIFDKHEVQEIKEKILNSDYDVEDFFEGEFFNFVLTQEGIDVYN | |
| | AIIGGFVTESGEKIKGLNEYINLYNAKTKQALPKFKPLYKQVLSDRES | |
| | LSFYGEGYTSDEEVLEVFRNTLNKNSEIFSSIKKLEKLFKNFDEYSSA | |
| | GIFVKNGPAISTISKDIFGEWNLIRDKWNAEYDDIHLKKKAVVTEKY | |
| | EDDRRKSFKKIGSFSLEQLQEYADADLSVVEKLKEIIIQKVDEIYKVY | |
| | GSSEKLFDADFVLEKSLKKNDAVVAIMKDLLDSVKSFENYIKAFFGE | |
| | GKETNRDESFYGDFVLAYDILLKVDHIYDAIRNYVTQKPYSKDKFKL | |
| | YFQNPQFMGGWDKDKETDYRATILRYGSKYYLAIMDKKYAKCLQK | |
| | IDKDDVNGNYEKINYKLLPGPNKMLPKVFFSKKWMAYYNPSEDIQK | |
| | IYKNGTFKKGDMFNLNDCHKLIDFFKDSISRYPKWSNAYDFNFSETE | |
| | KYKDIAGFYREVEEQGYKVSFESASKKEVDKLVEEGKLYMFQIYNK | |
| | DFSDKSHGTPNLHTMYFKLLFDENNHGQIRLSGGAELFMRRASLKK | |
| | EELVVHPANSPIANKNPDNPKKTTTLSYDVYKDKRFSEDQYELHIPIA | |
| | INKCPKNIFKINTEVRVLLKHDDNPYVIGIDRGERNLLYIVVVDGKGN | |
| | IVEQYSLNEIINNFNGIRIKTDYHSLLDKKEKERFEARQNWTSIENIKE | |
| | LKAGYISQVVHKICELVEKYDAVIALEDLNSGFKNSRVKVEKQVYQ | |
| | KFEKMLIDKLNYMVDKKSNPCATGGALKGYQITNKFESFKSMSTQN | |
| | GFIFYIPAWLTSKIDPSTGFVNLLKTKYTSIADSKKFISSFDRIMYVPEE | |
| | DLFEFALDYKNFSRTDADYIKKWKLYSYGNRIRIFAAAKKNNVFAW | |
| | EEVCLTSAYKELFNKYGINYQQGDIRALLCEQSDKAFYSSFMALMSL | |
| | MLQMRNSITGRTDVDFLISPVKNSDGIFYDSRNYEAQENAILPKNAD | |
| | ANGAYNIARKVLWAIGQFKKAEDEKLDKVKIAISNKEWLEYAQTSV | |
| | K | |
| FnCpf1 | MSIYQEFVNKYSLSKTLRFELIPQGKTLENIKARGLILDDEKRAKDYK | SEQ |
| | KAKQIIDKYHQFFIEEILSSVCISEDLLQNYSDVYFKLKKSDDDNLQK | ID |
| | DFKSAKDTIKKQISEYIKDSEKFKNLFNQNLIDAKKGQESDLILWLKQ | NO: |
| | SKDNGIELFKANSDITDIDEALEIIKSFKGWTTYFKGFHENRKNVYSS | 744 |
| | NDIPTSIIYRIVDDNLPKFLENKAKYESLKDKAPEAINYEQIKKDLAEE | |
| | LTFDIDYKTSEVNQRVFSLDEVFEIANFNNYLNQSGITKENTIIGGKFV | |
| | NGENTKRKGINEYINLYSQQINDKTLKKYKMSVLFKQILSDTESKSFV | |
| | IDKLEDDSDVVTTMQSFYEQIAAFKTVEEKSIKETLSLLFDDLKAQKL | |
| | DLSKIYFKNDKSLTDLSQQVEDDYSVIGTAVLEYITQQIAPKNLDNPS | |
| | KKEQELIAKKTEKAKYLSLETIKLALEEFNKHRDIDKQCRFEEILANF | |
| | AAIPMIFDEIAQNKDNLAQISIKYQNQGKKDLLQASAEDDVKAIKDL | |
| | LDQTNNLLHKLKIFHISQSEDKANILDKDEHFYLVFEECYFELANIVP | |
| | LYNKIRNYITQKPYSDEKFKLNFENSTLANGWDKNKEPDNTAILFIK | |
| | DDKYYLGVMNKKNNKIFDDKAIKENKGEGYKKIVYKLLPGANKML | |
| | PKVFFSAKSIKFYNPSEDILRIRNHSTHTKNGSPQKGYEKFEFNIEDCR | |
| | KFIDFYKQSISKHPEWKDFGFRFSDTQRYNSIDEFYREVENQGYKLTF | |
| | ENISESYIDSVVNQGKLYLFQIYNKDFSAYSKGRPNLHTLYWKALFD | |
| | ERNLQDVVYKLNGEAELFYRKQSIPKKITHPAKEAIANKNKDNPKKE | |
| | SVFEYDLIKDKRFTEDKFFFHCPITINFKSSGANKFNDEINLLLKEKAN | |
| | DVHILSIDRGERHLAYYTLVDGKGNIIKQDTFNIIGNDRMKTNYHDK | |
| | LAAIEKDRDSARKDWKKINNIKEMKEGYLSQVVHEIAKLVIEYNAIV | |
| | VFEDLNFGFKRGRFKVEKQVYQKLEKMLIEKLNYLVFKDNEFDKTG | |
| | GVLRAYQLTAPFETFKKMGKQTGIIYYVPAGFTSKICPVTGFVNQLY | |
| | PKYESVSKSQEFFSKFDKICYNLDKGYFEFSFDYKNFGDKAAKGKW | |
| | TIASFGSRLINFRNSDKNHNWDTREVYPTKELEKLLKDYSIEYGHGE | |
| | CIKAAICGESDKKFFAKLTSVLNTILQMRNSKTGTELDYLISPVADVN | |
| | GNFFDSRQAPKNMPQDADANGAYHIGLKGLMLLGRIKNNQEGKKL | |
| | NLVIKNEEYFEFVQNRNN | |
| VQRCas9 | MDKKYSIGLDIGTNSVGWAVITDEYKVPSKKFKVLGNTDRHSIKKNL | SEQ |
| | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 745 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFVSPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKQYRSTKEVLDATLIHQSITGLYETRIDLSQLGGD | |
| EQRCas9 | MDKKYSIGLDIGTNSVGWAVITDEYKVPSKKFKVLGNTDRHSIKKNL | SEQ |
| | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 746 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFESPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKQYRSTKEVLDATLIHQSITGLYETRIDLSQLGGD | |
| VRER | MDKKYSIGLDIGTNSVGWAVITDEYKVPSKKFKVLGNTDRHSIKKNL | SEQ |
| Cas9 | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 747 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFVSPTVAYSVLVVAKVEKGKSKKLKSVKELLGITIMERSSFEKNPI | |
| | DFLEAKGYKEVKKDLIIKLPKYSLFELENGRKRMLASARELQKGNEL | |
| Cas9- | ALPSKYVNFLYLASHYEKLKGSPEDNEQKQLFVEQHKHYLDEIIEQIS | SEQ |
| NG | EFSKRVILADANLDKVLSAYNKHRDKPIREQAENIIHLFTLTNLGAPA | ID |
| | AFKYFDTTIDRKEYRSTKEVLDATLIHQSITGLYETRIDLSQLGGD | NO: |
| | MDKKYSIGLDIGTNSVGWAVITDEYKVPSKKFKVLGNTDRHSIKKNL | 748 |
| | IGALLFDSGETAEATRLKRTARRRYTRRKNRICYLQEIFSNEMAKVD | |
| | DSFFHRLEESFLVEEDKKHERHPIFGNIVDEVAYHEKYPTIYHLRKKL | |
| | VDSTDKADLRLIYLALAHMIKFRGHFLIEGDLNPDNSDVDKLFIQLV | |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESIRPKRNSDKLIARKKDWDPKK | |
| | YGGFVSPTVAYSVLVVAKVEKGKSKKLKSVKELLGITIMERSSFEKN | |
| | PIDFLEAKGYKEVKKDLIIKLPKYSLFELENGRKRMLASARFLQKGN | |
| | ELALPSKYVNFLYLASHYEKLKGSPEDNEQKQLFVEQHKHYLDEIIE | |
| | QISEFSKRVILADANLDKVLSAYNKHRDKPIREQAENIIHLFTLTNLG | |
| | APRAFKYFDTTIDRKVYRSTKEVLDATLIHQSITGLYETRIDLSQLGG | |
| | D | |
| xCas9 | MDKKYSIGLDIGTNSVGWAVITDEYKVPSKKFKVLGNTDRHSIKKNL | SEQ |
| | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 749 |
| | QTYNQLFEENPINASGVDAKAILSARLSKSRRLENLIAQLPGEKKNGL | |
| | FGNLIALSLGLTPNFKSNFDLAEDTKLQLSKDTYDDDLDNLLAQIGD | |
| | QYADLFLAAKNLSDAILLSDILRVNTEITKAPLSASMIKLYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGIIPHQIHLGELHAILRRQED | |
| | FYPFLKDNREKIEKILTFRIPYYVGPLARGNSRFAWMTRKSEETITPW | |
| | NFEKVVDKGASAQSFIERMTNFDKNLPNEKVLPKHSLLYEYFTVYNE | |
| | LTKVKYVTEGMRKPAFLSGDQKKAIVDLLFKTNRKVTVKQLKEDYF | |
| | KKIECFDSVEISGVEDRFNASLGTYHDLLKIIKDKDFLDNEENEDILED | |
| | IVLTLTLFEDREMIEERLKTYAHLFDDKVMKQLKRRRYTGWGRLSR | |
| | KLINGIRDKQSGKTILDFLKSDGFANRNFIQLIHDDSLTFKEDIQKAQV | |
| | SGQGDSLHEHIANLAGSPAIKKGILQTVKVVDELVKVMGRHKPENIV | |
| | IEMARENQTTQKGQKNSRERMKRIEEGIKELGSQILKEHPVENTQLQ | |
| | NEKLYLYYLQNGRDMYVDQELDINRLSDYDVDHIVPQSFLKDDSID | |
| | NKVLTRSDKNRGKSDNVPSEEVVKKMKNYWRQLLNAKLITQRKFD | |
| | NLTKAERGGLSELDKAGFIKRQLVETRQITKHVAQILDSRMNTKYDE | |
| | NDKLIREVKVITLKSKLVSDFRKDFQFYKVREINNYHHAHDAYLNA | |
| | VVGTALIKKYPKLESEFVYGDYKVYDVRKMIAKSEQEIGKATAKYF | |
| | FYSNIMNFFKTEITLANGEIRKRPLIETNGETGEIVWDKGRDFATVRK | |
| | VLSMPQVNIVKKTEVQTGGFSKESILPKRNSDKLIARKKDWDPKKYG | |
| | GFDSPTVAYSVLVVAKVEKGKSKKLKSVKELLGITIMERSSFEKNPID | |
| | FLEAKGYKEVKKDLIIKLPKYSLFELENGRKRMLASAGVLQKGNELA | |
| | LPSKYVNFLYLASHYEKLKGSPEDNEQKQLFVEQHKHYLDEIIEQISE | |
| | FSKRVILADANLDKVLSAYNKHRDKPIREQAENIIHLFTLTNLGAPAA | |
| | FKYFDTTIDRKRYTSTKEVLDATLIHQSITGLYETRIDLSQLGGD | |
| eCas9 | MDKKYSIGLDIGTNSVGWAVITDEYKVPSKKFKVLGNTDRHSIKKNL | SEQ |
| | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 750 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLADD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPALESEFVYGDYKVYDVRKMIAKSEQEIGKATAKY | |
| | FFYSNIMNFFKTEITLANGEIRKAPLIETNGETGEIVWDKGRDFATVR | |
| | KVLSMPQVNIVKKTEVQTGGFSKESILPKRNSDKLIARKKDWDPKKY | |
| | GGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKRYTSTKEVLDATLIHQSITGLYETRIDLSQLGGD | |
| SpCas9- | MDKKYSIGLDIGTNSVGWAVITDEYKVPSKKFKVLGNTDRHSIKKNL | SEQ |
| HF1 | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 751 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTAFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGAL | |
| | SRKLINGIRDKQSGKTILDFLKSDGFANRNFMALIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRAITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKRYTSTKEVLDATLIHQSITGLYETRIDLSQLGGD | |
| HypaCas9 | MDKKYSIGLDIGTNSVGWAVITDEYKVPSKKFKVLGNTDRHSIKKNL | SEQ |
| | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 752 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRAFAALIADDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKRYTSTKEVLDATLIHQSITGLYETRIDLSQLGGD | |
| HiFiCas9 | MDKKYSIGLDIGTNSVGWAVITDEYKVPSKKFKVLGNTDRHSIKKNL | SEQ |
| | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 753 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANANFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFDSPTVAYSVLVVAKVEKGKSKKLKSVKELLGITIMERSSFEKNPI | |
| | DFLEAKGYKEVKKDLIIKLPKYSLFELENGRKRMLASAGELQKGNEL | |
| | ALPSKYVNFLYLASHYEKLKGSPEDNEQKQLFVEQHKHYLDEIIEQIS | |
| | EFSKRVILADANLDKVLSAYNKHRDKPIREQAENIIHLFTLTNLGAPA | |
| | AFKYFDTTIDRKRYTSTKEVLDATLIHQSITGLYETRIDLSQLGGD | |
| sniper- | MDKKYSIGLDIGTNSVGWAVITDEYKVPSKKFKVLGNTDRHSIKKNL | SEQ |
| Cas9 | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 754 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPASLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEIARENQTTQKGQKNSRERMKRIEEGIKELGSQILKEHPVENTQ | |
| | LQNEKLYLYYLQNGRDMYVDQELDINRLSDYDVDHIVPQSFLKDDS | |
| | IDNKVLTRSDKNRGKSDNVPSEEVVKKMKNYWRQLLNANLITQRKF | |
| | DNLTKAERGGLSELDKAGFIKRQLVETRQITKHVAQILDSRMNTKYD | |
| | ENDKLIREVKVITLKSKLVSDFRKDFQFYKVREINNYHHAHDAYLNA | |
| | VVGTALIKKYPKLESEFVYGDYKVYDVRKMIAKSEQEIGKATAKYF | |
| | FYSNIMNFFKTEITLANGEIRKRPLIETNGETGEIVWDKGRDFATVRK | |
| | VLSMPQVNIVKKTEVQTGGFSKESILPKRNSDKLIARKKDWDPKKYG | |
| | GFDSPTVAYSVLVVAKVEKGKSKKLKSVKELLGITIMERSSFEKNPID | |
| | FLEAKGYKEVKKDLIIKLPKYSLFELENGRKRMLASAGELQKGNELA | |
| | LPSKYVNFLYLASHYEKLKGSPEDNEQKQLFVEQHKHYLDEIIEQISE | |
| | FSKRVILADANLDKVLSAYNKHRDKPIREQAENIIHLFTLTNLGAPAA | |
| | FKYFDTTIDRKRYTSTKEVLDATLIHQSITGLYETRIDLSQLGGD | |
| spG | MDKKYSIGLDIGTNSVGWAVITDEYKVPSKKFKVLGNTDRHSIKKNL | SEQ |
| | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 755 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKRNSDKLIARKKDWDPKKY | |
| | GGFLWPTVAYSVLVVAKVEKGKSKKLKSVKELLGITIMERSSFEKNP | |
| | IDFLEAKGYKEVKKDLIIKLPKYSLFELENGRKRMLASAKQLQKGNE | |
| | LALPSKYVNFLYLASHYEKLKGSPEDNEQKQLFVEQHKHYLDEIIEQI | |
| | SEFSKRVILADANLDKVLSAYNKHRDKPIREQAENIIHLFTLTNLGAP | |
| | AAFKYFDTTIDRKQYRSTKEVLDATLIHQSITGLYETRIDLSQLGGD | |
| SpRY | MDKKYSIGLDIGTNSVGWAVITDEYKVPSKKFKVLGNTDRHSIKKNL | SEQ |
| KKH | IGALLFDSGETAERTRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 756 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMIKRYDEHHQDL | |
| | TLLKALVRQQLPEKYKEIFFDQSKNGYAGYIDGGASQEEFYKFIKPIL | |
| | EKMDGTEELLVKLNREDLLRKQRTFDNGSIPHQIHLGELHAILRRQE | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRRRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGRHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESIRPKRNSDKLIARKKDWDPKK | |
| | YGGFLWPTVAYSVLVVAKVEKGKSKKLKSVKELLGITIMERSSFEKN | |
| | PIDFLEAKGYKEVKKDLIIKLPKYSLFELENGRKRMLASAKQLQKGN | |
| | ELALPSKYVNFLYLASHYEKLKGSPEDNEQKQLFVEQHKHYLDEIIE | |
| | QISEFSKRVILADANLDKVLSAYNKHRDKPIREQAENIIHLFTLTRLG | |
| | APRAFKYFDTTIDPKQYRSTKEVLDATLIHQSITGLYETRIDLSQLGG | |
| | D | |
| SaCas9 | MGKRNYILGLDIGITSVGYGIIDYETRDVIDAGVRLFKEANVENNEGR | SEQ |
| | RSKRGARRLKRRRRHRIQRVKKLLFDYNLLTDHSELSGINPYEARVK | ID |
| | GLSQKLSEEEFSAALLHLAKRRGVHNVNEVEEDTGNELSTKEQISRN | NO: |
| | SKALEEKYVAELQLERLKKDGEVRGSINRFKTSDYVKEAKQLLKVQ | 757 |
| | KAYHQLDQSFIDTYIDLLETRRTYYEGPGEGSPFGWKDIKEWYEML | |
| | MGHCTYFPEELRSVKYAYNADLYNALNDLNNLVITRDENEKLEYYE | |
| | KFQIIENVFKQKKKPTLKQIAKEILVNEEDIKGYRVTSTGKPEFTNLK | |
| | VYHDIKDITARKEIIENAELLDQIAKILTIYQSSEDIQEELTNLNSELTQ | |
| | EEIEQISNLKGYTGTHNLSLKAINLILDELWHTNDNQIAIFNRLKLVPK | |
| | KVDLSQQKEIPTTLVDDFILSPVVKRSFIQSIKVINAIIKKYGLPNDIIIE | |
| | LAREKNSKDAQKMINEMQKRNRQTNERIEEIIRTTGKENAKYLIEKIK | |
| | LHDMQEGKCLYSLEAIPLEDLLNNPFNYEVDHIIPRSVSFDNSFNNKV | |
| | LVKQEENSKKGNRTPFQYLSSSDSKISYETFKKHILNLAKGKGRISKT | |
| | KKEYLLEERDINRFSVQKDFINRNLVDTRYATRGLMNLLRSYFRVNN | |
| | LDVKVKSINGGFTSFLRRKWKFKKERNKGYKHHAEDALIIANADFIF | |
| | KEWKKLDKAKKVMENQMFEEKQAESMPEIETEQEYKEIFITPHQIKH | |
| | IKDFKDYKYSHRVDKKPNRKLINDTLYSTRKDDKGNTLIVNNLNGL | |
| | YDKDNDKLKKLINKSPEKLLMYHHDPQTYQKLKLIMEQYGDEKNPL | |
| | YKYYEETGNYLTKYSKKDNGPVIKKIKYYGNKLNAHLDITDDYPNS | |
| | RNKVVKLSLKPYRFDVYLDNGVYKFVTVKNLDVIKKENYYEVNSK | |
| | CYEEAKKLKKISNQAEFIASFYKNDLIKINGELYRVIGVNNDLLNRIE | |
| | VNMIDITYREYLENMNDKRPPHIIKTIASKTQSIKKYSTDILGNLYEVK | |
| | SKKHPQIIKKG | |
| CjCas9 | MARILAFDIGISSIGWAFSENDELKDCGVRIFTKVENPKTGESLALPRR | SEQ |
| | LARSARKRLARRKARLNHLKHLIANEFKLNYEDYQSFDESLAKAYK | ID |
| | GSLISPYELRFRALNELLSKQDFARVILHIAKRRGYDDIKNSDDKEKG | NO: |
| | AILKAIKQNEEKLANYQSVGEYLYKEYFQKFKENSKEFTNVRNKKES | 758 |
| | YERCIAQSFLKDELKLIFKKQREFGFSFSKKFEEEVLSVAFYKRALKD | |
| | FSHLVGNCSFFTDEKRAPKNSPLAFMFVALTRIINLLNNLKNTEGILY | |
| | TKDDLNALLNEVLKNGTLTYKQTKKLLGLSDDYEFKGEKGTYFIEF | |
| | KKYKEFIKALGEHNLSQDDLNEIAKDITLIKDEIKLKKALAKYDLNQ | |
| | NQIDSLSKLEFKDHLNISFKALKLVTPLMLEGKKYDEACNELNLKVA | |
| | INEDKKDFLPAFNETYYKDEVTNPVVLRAIKEYRKVLNALLKKYGK | |
| | VHKINIELAREVGKNHSQRAKIEKEQNENYKAKKDAELECEKLGLKI | |
| | NSKNILKLRLFKEQKEFCAYSGEKIKISDLQDEKMLEIDHIYPYSRSFD | |
| | DSYMNKVLVFTKQNQEKLNQTPFEAFGNDSAKWQKIEVLAKNLPTK | |
| | KQKRILDKNYKDKEQKNFKDRNLNDTRYIARLVLNYTKDYLDFLPL | |
| | SDDENTKLNDTQKGSKVHVEAKSGMLTSALRHTWGFSAKDRNNHL | |
| | HHAIDAVIIAYANNSIVKAFSDFKKEQESNSAELYAKKISELDYKNKR | |
| | KFFEPFSGFRQKVLDKIDEIFVSKPERKKPSGALHEETFRKEEEFYQSY | |
| | GGKEGVLKALELGKIRKVNGKIVKNGDMFRVDIFKHKKTNKFYAVP | |
| | IYTMDFALKVLPNKAVARSKKGEIKDWILMDENYEFCFSLYKDSLILI | |
| | QTKDMQEPEFVYYNAFTSSTVSLIVSKHDNKFETLSKNQKILFKNAN | |
| | EKEVIAKSIGIQNLKVFEKYIVSALGEVTKAEFRQREDFKK | |
| Cas9- | MDKKYSIGLDIGTNSVGWAVITDEYKVPSKKFKVLGNTDRHSIKKNL | SEQ |
| NRRH | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 759 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMVKRYDEHHQD | |
| | LTLLKALVRQQLPEKYKEIFFDQSKNGYAGYIDGGASQEEFYKFIKPI | |
| | LEKMDGTEELLVKLNREDLLRKQRTFDNGIIPHQIHLGELHAILRRQG | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRLRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGGHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKGNSDKLIARKKDWDPKK | |
| | YGGFNSPTAAYSVLVVAKVEKGKSKKLKSVKELLGITIMERSSFEKN | |
| | PIGFLEAKGYKEVKKDLIIKLPKYSLFELENGRKRMLASAGVLHKGN | |
| | ELALPSKYVNFLYLASHYEKLKGSPEDNEQKQLFVEQHKHYLDEIIE | |
| | QISEFSKRVILADANLDKVLSAYNKHRDKPIREQAENIIHLFTLTNLG | |
| | VPAAFKYFDTTIDKKRYTSTKEVLDATLIHQSITGLYETRIDLSQLGG | |
| | D | |
| Cas9- | MDKKYSIGLDIGTNSVGWAVITDEYKVPSKKFKVLGNTDRHSIKKNL | SEQ |
| NRCH | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 760 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMVKRYDEHHQD | |
| | LTLLKALVRQQLPEKYKEIFFDQSKNGYAGYIDGGASQEEFYKFIKPI | |
| | LEKMDGTEELLVKLNREDLLRKQRTFDNGIIPHQIHLGELHAILRRQG | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRLRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGGHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKGNSDKLIARKKDWDPKK | |
| | YGGFNSPTVAYSVLVVAKVEKGKSKKLKSVKELLGITIMERSSFEKN | |
| | PIDFLEAKGYKEVKKDLIIKLPKYSLFELENGRKRMLASAGVLQKGN | |
| | ELALPSKYVNFLYLASHYEKLKGSPEDNEQKQLFVEQHKHYLDEIIE | |
| | QISEFSKRVILADANLDKVLSAYNKHRDKPIREQAENIIHLFTLTNLG | |
| | APAAFKYFDTTINRKQYNTTKEVLDATLIRQSITGLYETRIDLSQLGG | |
| | D | |
| Cas9- | MDKKYSIGLDIGTNSVGWAVITDEYKVPSKKFKVLGNTDRHSIKKNL | SEQ |
| NRTH | IGALLFDSGETAEATRLKRTARRRYTRRKNRICYLQEIFSNEMAKVD | ID |
| | DSFFHRLEESFLVEEDKKHERHPIFGNIVDEVAYHEKYPTIYHLRKKL | NO: |
| | VDSTDKADLRLIYLALAHMIKFRGHFLIEGDLNPDNSDVDKLFIQLV | 761 |
| | QTYNQLFEENPINASGVDAKAILSARLSKSRRLENLIAQLPGEKKNGL | |
| | FGNLIALSLGLTPNFKSNFDLAEDAKLQLSKDTYDDDLDNLLAQIGD | |
| | QYADLFLAAKNLSDAILLSDILRVNTEITKAPLSASMVKRYDEHHQD | |
| | LTLLKALVRQQLPEKYKEIFFDQSKNGYAGYIDGGASQEEFYKFIKPI | |
| | LEKMDGTEELLVKLNREDLLRKQRTFDNGIIPHQIHLGELHAILRRQG | |
| | DFYPFLKDNREKIEKILTFRIPYYVGPLARGNSRFAWMTRKSEETITP | |
| | WNFEEVVDKGASAQSFIERMTNFDKNLPNEKVLPKHSLLYEYFTVY | |
| | NELTKVKYVTEGMRKPAFLSGEQKKAIVDLLFKTNRKVTVKQLKED | |
| | YFKKIECFDSVEISGVEDRFNASLGTYHDLLKIIKDKDFLDNEENEDIL | |
| | EDIVLTLTLFEDREMIEERLKTYAHLFDDKVMKQLKRLRYTGWGRL | |
| | SRKLINGIRDKQSGKTILDFLKSDGFANRNFMQLIHDDSLTFKEDIQK | |
| | AQVSGQGDSLHEHIANLAGSPAIKKGILQTVKVVDELVKVMGGHKP | |
| | ENIVIEMARENQTTQKGQKNSRERMKRIEEGIKELGSQILKEHPVENT | |
| | QLQNEKLYLYYLQNGRDMYVDQELDINRLSDYDVDHIVPQSFLKDD | |
| | SIDNKVLTRSDKNRGKSDNVPSEEVVKKMKNYWRQLLNAKLITQRK | |
| | FDNLTKAERGGLSELDKAGFIKRQLVETRQITKHVAQILDSRMNTKY | |
| | DENDKLIREVKVITLKSKLVSDFRKDFQFYKVREINNYHHAHDAYLN | |
| | AVVGTALIKKYPKLESEFVYGDYKVYDVRKMIAKSEQEIGKATAKY | |
| | FFYSNIMNFFKTEITLANGEIRKRPLIETNGETGEIVWDKGRDFATVR | |
| | KVLSMPQVNIVKKTEVQTGGFSKESILPKGNSDKLIARKKDWDPKK | |
| | YGGFNSPTVAYSVLVVAKVEKGKSKKLKSVKELLGITIMERSSFEKN | |
| | PIGFLEAKGYKEVKKDLIIKLPKYSLFELENGRKRMLASASVLHKGN | |
| | ELALPSKYVNFLYLASHYEKLKGSSEDNKQKQLFVEQHKHYLDEIIE | |
| | QISEFSKRVILADANLDKVLSAYNKHRDKPIREQAENIIHLFTLTNLG | |
| | ASAAFKYFDTTIGRKLYTSTKEVLDATLIHQSITGLYETRIDLSQLGG | |
| | D | |
| ScCpf1 | MQTLFENFTNQYPVSKTLRFELIPQGKTKDFIEQKGLLKKDEDRAEK | SEQ |
| | YKKVKNIIDEYHKDFIEKSLNGLKLDGLEKYKTLYLKQEKDDKDKK | ID |
| | AFDKEKENLRKQIANAFRNNEKFKTLFAKELIKNDLMSFACEEDKKN | NO: |
| | VKEFEAFTTYFTGFHQNRANMYVADEKRTAIASRLIHENLPKFIDNIK | 762 |
| | IFEKMKKEAPELLSPFNQTLKDMKDVIKGTTLEEIFSLDYFNKTLTQS | |
| | GIDIYNSVIGGRTPEEGKTKIKGLNEYINTDFNQKQTDKKKRQPKFKQ | |
| | LYKQILSDRQSLSFIAEAFKNDTEILEAIEKFYVNELLHFSNEGKSTNV | |
| | LDAIKNAVSNLESFNLTKMYFRSGASLTDVSRKVFGEWSIINRALDN | |
| | YYATTYPIKPREKSEKYEERKEKWLKQDFNVSLIQTAIDEYDNETVK | |
| | GKNSGKVIADYFAKFCDDKETDLIQKVNEGYIAVKDLLNTPCPENEK | |
| | LGSNKDQVKQIKAFMDSIMDIMHFVRPLSLKDTDKEKDETFYSLFTP | |
| | LYDHLTQTIALYNKVRNYLTQKPYSTEKIKLNFENSTLLGGWDLNKE | |
| | TDNTAIILRKDNLYYLGIMDKRHNRIFRNVPKADKKDFCYEKMVYK | |
| | LLPGANKMLPKVFFSQSRIQEFTPSAKLLENYANETHKKGDNFNLNH | |
| | CHKLIDFFKDSINKHEDWKNFDFRFSATSTYADLSGFYHEVEHQGYK | |
| | ISFQSVADSFIDDLVNEGKLYLFQIYNKDFSPFSKGKPNLHTLYWKM | |
| | LFDENNLKDVVYKLNGEAEVFYRKKSIAEKNTTIHKANESIINKNPD | |
| | NPKATSTFNYDIVKDKRYTIDKFQFHIPITMNFKAEGIFNMNQRVNQF | |
| | LKANPDINIIGIDRGERHLLYYALINQKGKILKQDTLNVIANEKQKVD | |
| | YHNLLDKKEGDRATARQEWGVIETIKELKEGYLSQVIHKLTDLMIEN | |
| | NAIIVMEDLNFGFKRGRQKVEKQVYQKFEKMLIDKLNYLVDKNKK | |
| | ANELGGLLNAFQLANKFESFQKMGKQNGFIFYVPAWNTSKTDPATG | |
| | FIDFLKPRYENLNQAKDFFEKFDSIRLNSKADYFEFAFDFKNFTEKAD | |
| | GGRTKWTVCTTNEDRYAWNRALNNNRGSQEKYDITAELKSLFDGK | |
| | VDYKSGKDLKQQIASQESADFFKALMKNLSITLSLRHNNGEKGDNE | |
| | QDYILSPVADSKGRFFDSRKADDDMPKNADANGAYHIALKGLWCLE | |
| | QISKTDDLKKVKLAISNKEWLEFVQTLKG | |
| PcCpfl | MDSLKDFTNLYPVSKTLRFELKPVGKTLENIEKAGILKEDEHRAESY | SEQ |
| | RRVKKIIDTYHKVFIDSSLENMAKMGIENEIKAMLQSFCELYKKDHR | ID |
| | TEGEDKALDKIRAVLRGLIVGAFTGVCGRRENTVQNEKYESLFKEKL | NO: |
| | IKEILPDFVLSTEAESLPFSVEEATRSLKEFDSFTSYFAGFYENRKNIYS | 763 |
| | TKPQSTAIAYRLIHENLPKFIDNILVFQKIKEPIAKELEHIRADFSAGGY | |
| | IKKDERLEDIFSLNYYIHVLSQAGIEKYNALIGKIVTEGDGEMKGLNE | |
| | HINLYNQQRGREDRLPLFRPLYKQILSDREQLSYLPESFEKDEELLRA | |
| | LKEFYDHIAEDILGRTQQLMTSISEYDLSRIYVRNDSQLTDISKKMLG | |
| | DWNAIYMARERAYDHEQAPKRITAKYERDRIKALKGEESISLANLNS | |
| | CIAFLDNVRDCRVDTYLSTLGQKEGPHGLSNLVENVFASYHEAEQLL | |
| | SFPYPEENNLIQDKDNVVLIKNLLDNISDLQRFLKPLWGMGDEPDKD | |
| | ERFYGEYNYIRGALDQVIPLYNKVRNYLTRKPYSTRKVKLNFGNSQL | |
| | LSGWDRNKEKDNSCVILRKGQNFYLAIMNNRHKRSFENKMLPEYKE | |
| | GEPYFEKMDYKFLPDPNKMLPKVFLSKKGIEIYKPSPKLLEQYGHGT | |
| | HKKGDTFSMDDLHELIDFFKHSIEAHEDWKQFGFKFSDTATYENVSS | |
| | FYREVEDQGYKLSFRKVSESYVYSLIDQGKLYLFQIYNKDFSPCSKG | |
| | TPNLHTLYWRMLFDERNLADVIYKLDGKAEIFFREKSLKNDHPTHPA | |
| | GKPIKKKSRQKKGEESLFEYDLVKDRRYTMDKFQFHVPITMNFKCS | |
| | AGSKVNDMVNAHIREAKDMHVIGIDRGERNLLYICVIDSRGTILDQIS | |
| | LNTINDIDYHDLLESRDKDRQQEHRNWQTIEGIKELKQGYLSQAVHR | |
| | IAELMVAYKAVVALEDLNMGFKRGRQKVESSVYQQFEKQLIDKLN | |
| | YLVDKKKRPEDIGGLLRAYQFTAPFKSFKEMGKQNGFLFYIPAWNTS | |
| | NIDPTTGFVNLFHVQYENVDKAKSFFQKFDSISYNPKKDWFEFAFDY | |
| | KNFTKKAEGSRSMWILCTHGSRIKNFRNSQKNGQWDSEEFALTEAF | |
| | KSLFVRYEIDYTADLKTAIVDEKQKDFFVDLLKLFKLTVQMRNSWK | |
| | EKDLDYLISPVAGADGRFFDTREGNKSLPKDADANGAYNIALKGLW | |
| | ALRQIRQTSEGGKLKLAISNKEWLQFVQERSYEKD | |
| BpCpf1 | MLLYENYTKRNQITKSLRLELRPQGKTLRNIKELNLLEQDKAIYALL | SEQ |
| | ERLKPVIDEGIKDIARDTLKNCELSFEKLYEHFLSGDKKAYAKESERL | ID |
| | KKEIVKTLIKNLPEGIGKISEINSAKYLNGVLYDFIDKTHKDSEEKQNI | NO: |
| | LSDILETKGYLALFSKFLTSRITTLEQSMPKRVIENFEIYAANIPKMQD | 764 |
| | ALERGAVSFAIEYESICSVDYYNQILSQEDIDSYNRLISGIMDEDGAKE | |
| | KGINQTISEKNIKIKSEHLEEKPFRILKQLHKQILEEREKAFTIDHIDSD | |
| | EEVVQVTKEAFEQTKEQWENIKKINGFYAKDPGDITLFIVVGPNQTH | |
| | VLSQLIYGEHDRIRLLLEEYEKNTLEVLPRRTKSEKARYDKFVNAVP | |
| | KKVAKESHTFDGLQKMTGDDRLFILYRDELARNYMRIKEAYGTFER | |
| | DILKSRRGIKGNRDVQESLVSFYDELTKFRSALRIINSGNDEKADPIFY | |
| | NTFDGIFEKANRTYKAENLCRNYVTKSPADDARIMASCLGTPARLRT | |
| | HWWNGEENFAINDVAMIRRGDEYYYFVLTPDVKPVDLKTKDETDA | |
| | QIFVQRKGAKSFLGLPKALFKCILEPYFESPEHKNDKNCVIEEYVSKP | |
| | LTIDRRAYDIFKNGTFKKTNIGIDGLTEEKFKDDCRYLIDVYKEFIAV | |
| | YTRYSCFNMSGLKRADEYNDIGEFFSDVDTRLCTMEWIPVSFERIND | |
| | MVDKKEGLLFLVRSMFLYNRPRKPYERTFIQLFSDSNMEHTSMLLNS | |
| | RAMIQYRAASLPRRVTHKKGSILVALRDSNGEHIPMHIREAIYKMKN | |
| | NFDISSEDFIMAKAYLAEHDVAIKKANEDIIRNRRYTEDKFFLSLSYT | |
| | KNADISARTLDYINDKVEEDTQDSRMAVIVTRNLKDLTYVAVVDEK | |
| | NNVLEEKSLNEIDGVNYRELLKERTKIKYHDKTRLWQYDVSSKGLK | |
| | EAYVELAVTQISKLATKYNAVVVVESMSSTFKDKFSFLDEQIFKAFE | |
| | ARLCARMSDLSFNTIKEGEAGSISNPIQVSNNNGNSYQDGVIYFLNNA | |
| | YTRTLCPDTGFVDVFDKTRLITMQSKRQFFAKMKDIRIDDGEMLFTF | |
| | NLEEYPTKRLLDRKEWTVKIAGDGSYFDKDKGEYVYVNDIVREQIIP | |
| | ALLEDKAVFDGNMAEKFLDKTAISGKSVELIYKWFANALYGIITKKD | |
| | GEKIYRSPITGTEIDVSKNTTYNFGKKFMFKQEYRGDGDFLDAFLNY | |
| | MQAQDIAV | |
| LiCpf1 | MEDYSGFVNIYSIQKTLRFELKPVGKTLEHIEKKGFLKKDKIRAEDYK | SEQ |
| | AVKKIIDKYHRAYIEEVFDSVLHQKKKKDKTRFSTQFIKEIKEFSELY | ID |
| | YKTEKNIPDKERLEALSEKLRKMLVGAFKGEFSEEVAEKYKNLFSKE | NO: |
| | LIRNEIEKFCETDEERKQVSNFKSFTTYFTGFHSNRQNIYSDEKKSTAI | 765 |
| | GYRIIHQNLPKFLDNLKIIESIQRRFKDFPWSDLKKNLKKIDKNIKLTE | |
| | YFSIDGFVNVLNQKGIDAYNTILGGKSEESGEKIQGLNEYINLYRQKN | |
| | NIDRKNLPNVKILFKQILGDRETKSFIPEAFPDDQSVLNSITEFAKYLK | |
| | LDKKKKSIIAELKKFLSSFNRYELDGIYLANDNSLASISTFLFDDWSFI | |
| | KKSVSFKYDESVGDPKKKIKSPLKYEKEKEKWLKQKYYTISFLNDAI | |
| | ESYSKSQDEKRVKIRLEAYFAEFKSKDDAKKQFDLLERIEEAYAIVEP | |
| | LLGAEYPRDRNLKADKKEVGKIKDFLDSIKSLQFFLKPLLSAEIFDEK | |
| | DLGFYNQLEGYYEEIDSIGHLYNKVRNYLTGKIYSKEKFKLNFENST | |
| | LLKGWDENREVANLCVIFREDQKYYLGVMDKENNTILSDIPKVKPN | |
| | ELFYEKMVYKLIPTPHMQLPRIIFSSDNLSIYNPSKSILKIREAKSFKEG | |
| | KNFKLKDCHKFIDFYKESISKNEDWSRFDFKFSKTSSYENISEFYREV | |
| | ERQGYNLDFKKVSKFYIDSLVEDGKLYLFQIYNKDFSIFSKGKPNLHT | |
| | IYFRSLFSKENLKDVCLKLNGEAEMFFRKKSINYDEKKKREGHHPEL | |
| | FEKLKYPILKDKRYSEDKFQFHLPISLNFKSKERLNFNLKVNEFLKRN | |
| | KDINIIGIDRGERNLLYLVMINQKGEILKQTLLDSMQSGKGRPEINYK | |
| | EKLQEKEIERDKARKSWGTVENIKELKEGYLSIVIHQISKLMVENNAI | |
| | VVLEDLNIGFKRGRQKVERQVYQKFEKMLIDKLNFLVFKENKPTEPG | |
| | GVLKAYQLTDEFQSFEKLSKQTGFLFYVPSWNTSKIDPRTGFIDFLHP | |
| | AYENIEKAKQWINKFDSIRFNSKMDWFEFTADTRKFSENLMLGKNR | |
| | VWVICTTNVERYFTSKTANSSIQYNSIQITEKLKELFVDIPFSNGQDLK | |
| | PEILRKNDAVFFKSLLFYIKTTLSLRQNNGKKGEEEKDFILSPVVDSK | |
| | GRFFNSLEASDDEPKDADANGAYHIALKGLMNLLVLNETKEENLSR | |
| | PKWKIKNKDWLEFVWERNR | |
| PmCpf1 | MKTQHFFEDFTSLYSLSKTIRFELKPIGKTLENIKKNGLIRRDEQRLDD | SEQ |
| | YEKLKKVIDEYHEDFIANILSSFSFSEEILQSYIQNLSESEARAKIEKTM | ID |
| | RDTLAKAFSEDERYKSIFKKELVKKDIPVWCPAYKSLCKKFDNFTTS | NO: |
| | LVPFHENRKNLYTSNEITASIPYRIVHVNLPKFIQNIEALCELQKKMG | 766 |
| | ADLYLEMMENLRNVWPSFVKTPDDLCNLKTYNHLMVQSSISEYNRF | |
| | VGGYSTEDGTKHQGINEWINIYRQRNKEMRLPGLVFLHKQILAKVDS | |
| | SSFISDTLENDDQVFCVLRQFRKLFWNTVSSKEDDAASLKDLFCGLS | |
| | GYDPEAIYVSDAHLATISKNIFDRWNYISDAIRRKTEVLMPRKKESVE | |
| | RYAEKISKQIKKRQSYSLAELDDLLAHYSEESLPAGFSLLSYFTSLGG | |
| | QKYLVSDGEVILYEEGSNIWDEVLIAFRDLQVILDKDFTEKKLGKDE | |
| | EAVSVIKKALDSALRLRKFFDLLSGTGAEIRRDSSFYALYTDRMDKL | |
| | KGLLKMYDKVRNYLTKKPYSIEKFKLHFDNPSLLSGWDKNKELNNL | |
| | SVIFRQNGYYYLGIMTPKGKNLFKTLPKLGAEEMFYEKMEYKQIAEP | |
| | MLMLPKVFFPKKTKPAFAPDQSVVDIYNKKTFKTGQKGFNKKDLYR | |
| | LIDFYKEALTVHEWKLFNFSFSPTEQYRNIGEFFDEVREQAYKVSMV | |
| | NVPASYIDEAVENGKLYLFQIYNKDFSPYSKGIPNLHTLYWKALFSE | |
| | QNQSRVYKLCGGGELFYRKASLHMQDTTVHPKGISIHKKNLNKKGE | |
| | TSLFNYDLVKDKRFTEDKFFFHVPISINYKNKKITNVNQMVRDYIAQ | |
| | NDDLQIIGIDRGERNLLYISRIDTRGNLLEQFSLNVIESDKGDLRTDYQ | |
| | KILGDREQERLRRRQEWKSIESIKDLKDGYMSQVVHKICNMVVEHK | |
| | AIVVLENLNLSFMKGRKKVEKSVYEKFERMLVDKLNYLVVDKKNL | |
| | SNEPGGLYAAYQLTNPLFSFEELHRYPQSGILFFVDPWNTSLTDPSTG | |
| | FVNLLGRINYTNVGDARKFFDRFNAIRYDGKGNILFDLDLSRFDVRV | |
| | ETQRKLWTLTTFGSRIAKSKKSGKWMVERIENLSLCFLELFEQFNIGY | |
| | RVEKDLKKAILSQDRKEFYVRLIYLFNLMMQIRNSDGEEDYILSPAL | |
| | NEKNLQFDSRLIEAKDLPVDADANGAYNVARKGLMVVQRIKRGDH | |
| | ESIHRIGRAQWLRYVQEGIVE | |
| Lb2Cpf1 | MYYESLTKQYPVSKTIRNELIPIGKTLDNIRQNNILESDVKRKQNYEH | SEQ |
| | VKGILDEYHKQLINEALDNCTLPSLKIAAEIYLKNQKEVSDREDENKT | ID |
| | QDLLRKEVVEKLKAHENFTKIGKKDILDLLEKLPSISEDDYNALESFR | NO: |
| | NFYTYFTSYNKVRENLYSDKEKSSTVAYRLINENFPKFLDNVKSYRF | 767 |
| | VKTAGILADGLGEEEQDSLFIVETENKTLTQDGIDTYNSQVGKINSSI | |
| | NLYNQKNQKANGFRKIPKMKMLYKQILSDREESFIDEFQSDEVLIDN | |
| | VESYGSVLIESLKSSKVSAFFDALRESKGKNVYVKNDLAKTAMSNIV | |
| | FENWRTFDDLLNQEYDLANENKKKDDKYFEKRQKELKKNKSYSLE | |
| | HLCNLSEDSCNLIENYIHQISDDIENIIINNETFLRIVINEHDRSRKLAK | |
| | NRKAVKAIKDFLDSIKVLERELKLINSSGQELEKDLIVYSAHEELLVE | |
| | LKQVDSLYNMTRNYLTKKPFSTEKVKLNFNRSTLLNGWDRNKETD | |
| | NLGVLLLKDGKYYLGIMNTSANKAFVNPPVAKTEKVFKKVDYKLLP | |
| | VPNQMLPKVFFAKSNIDFYNPSSEIYSNYKKGTHKKGNMFSLEDCHN | |
| | LIDFFKESISKHEDWSKFGFKFSDTASYNDISEFYREVEKQGYKLTYT | |
| | DIDETYINDLIERNELYLFQIYNKDFSMYSKGKLNLHTLYFMMLFDQ | |
| | RNIDDVVYKLNGEAEVFYRPASISEDELIIHKAGEEIKNKNPNRARTK | |
| | ETSTFSYDIVKDKRYSKDKFTLHIPITMNFGVDEVKRENDAVNSAIRI | |
| | DENVNVIGIDRGERNLLYVVVIDSKGNILEQISLNSIINKEYDIETDYH | |
| | ALLDEREGGRDKARKDWNTVENIRDLKAGYLSQVVNVVAKLVLKY | |
| | NAIICLEDLNFGFKRGRQKVEKQVYQKFEKMLIDKLNYL VIDKSREQ | |
| | TSPKELGGALNALQLTSKFKSFKELGKQSGVIYYVPAYLTSKIDPTTG | |
| | FANLFYMKCENVEKSKRFFDGFDFIRFNALENVFEFGFDYRSFTQRA | |
| | CGINSKWTVCTNGERIIKYRNPDKNNMFDEKVVVVTDEMKNLFEQY | |
| | KIPYEDGRNVKDMIISNEEAEFYRRLYRLLQQTLQMRNSTSDGTRDY | |
| | IISPVKNKREAYFNSELSDGSVPKDADANGAYNIARKGLWVLEQIRQ | |
| | KSEGEKINLAMTNAEWLEYAQTHLL | |
| PbCpf1 | MENIFDQFIGKYSLSKTLRFELKPVGKTEDFLKINKVFEKDQTIDDSY | SEQ |
| | NQAKFYFDSLHQKFIDAALASDKTSELSFQNFADVLEKQNKIILDKK | ID |
| | REMGALRKRDKNAVGIDRLQKEINDAEDIIQKEKEKIYKDVRTLFDN | NO: |
| | EAESWKTYYQEREVDGKKITFSKADLKQKGADFLTAAGILKVLKYE | 768 |
| | FPEEKEKEFQAKNQPSLFVEEKENPGQKRYIFDSFDKFAGYLTKFQQ | |
| | TKKNLYAADGTSTAVATRIADNFIIFHQNTKVFRDKYKNNHTDLGFD | |
| | EENIFEIERYKNCLLQREIEHIKNENSYNKIIGRINKKIKEYRDQKAKD | |
| | TKLTKSDFPFFKNLDKQILGEVEKEKQLIEKTREKTEEDVLIERFKEFI | |
| | ENNEERFTAAKKLMNAFCNGEFESEYEGIYLKNKAINTISRRWFVSD | |
| | RDFELKLPQQKSKNKSEKNEPKVKKFISIAEIKNAVEELDGDIFKAVF | |
| | YDKKIIAQGGSKLEQFLVIWKYEFEYLFRDIERENGEKLLGYDSCLKI | |
| | AKQLGIFPQEKEAREKATAVIKNYADAGLGIFQMMKYFSLDDKDRK | |
| | NTPGQLSTNFYAEYDGYYKDFEFIKYYNEFRNFITKKPFDEDKIKLNF | |
| | ENGALLKGWDENKEYDFMGVILKKEGRLYLGIMHKNHRKLFQSMG | |
| | NAKGDNANRYQKMIYKQIADASKDVPRLLLTSKKAMEKFKPSQEIL | |
| | RIKKEKTFKRESKNFSLRDLHALIEYYRNCIPQYSNWSFYDFQFQDTG | |
| | KYQNIKEFTDDVQKYGYKISFRDIDDEYINQALNEGKMYLFEVVNK | |
| | DIYNTKNGSKNLHTLYFEHILSAENLNDPVFKLSGMAEIFQRQPSVNE | |
| | REKITTQKNQCILDKGDRAYKYRRYTEKKIMFHMSLVLNTGKGEIK | |
| | QVQFNKIINQRISSSDNEMRVNVIGIDRGEKNLLYYSVVKQNGEIIEQ | |
| | ASLNEINGVNYRDKLIEREKERLKNRQSWKPVVKIKDLKKGYISHVI | |
| | HKICQLIEKYSAIVVLEDLNMRFKQIRGGIERSVYQQFEKALIDKLGY | |
| | LVFKDNRDLRAPGGVLNGYQLSAPFVSFEKMRKQTGILFYTQAEYTS | |
| | KTDPITGFRKNVYISNSASLDKIKEAVKKFDAIGWDGKEQSYFFKYN | |
| | PYNLADEKYKNSTVSKEWAIFASAPRIRRQKGEDGYWKYDRVKVN | |
| | EEFEKLLKVWNFVNPKATDIKQEIIKKEKAGDLQGEKELDGRLRNF | |
| | WHSFIYLFNLVLELRNSFSLQIKIKAGEVIAVDEGVDFIASPVKPFFTT | |
| | PNPYIPSNLCWLAVENADANGAYNIARKGVMILKKIREHAKKDPEFK | |
| | KLPNLFISNAEWDEAARDWGKYAGTTALNLDH | |
| PeCpf1 | MSNFFKNFTNLYELSKTLRFELKPVGDTLTNMKDHLEYDEKLQTFL | SEQ |
| | KDQNIDDAYQALKPQFDEIHEEFITDSLESKKAKEIDFSEYLDLFQEK | ID |
| | KELNDSEKKLRNKIGETFNKAGEKWKKEKYPQYEWKKGSKIANGA | NO: |
| | DILSCQDMLQFIKYKNPEDEKIKNYIDDTLKGFFTYFGGFNQNRANY | 769 |
| | YETKKEASTAVATRIVHENLPKFCDNVIQFKHIIKRKKDGTVEKTER | |
| | KTEYLNAYQYLKNNNKITQIKDAETEKMIESTPIAEKIFDVYYFSSCL | |
| | SQKQIEEYNRIIGHYNLLINLYNQAKRSEGKHLSANEKKYKDLPKFK | |
| | TLYKQIGCGKKKDLFYTIKCDTEEEANKSRNEGKESHSVEEIINKAQE | |
| | AINKYFKSNNDCENINTVPDFINYILTKENYEGVYWSKAAMNTISDK | |
| | YFANYHDLQDRLKEAKVFQKADKKSEDDIKIPEAIELSGLFGVLDSL | |
| | ADWQTTLFKSSILSNEDKLKIITDSQTPSEALLKMIFNDIEKNMESFLK | |
| | ETNDIITLKKYKGNKEGTEKIKQWFDYTLAINRMLKYFLVKENKIKG | |
| | NSLDTNISEALKTLIYSDDAEWFKWYDALRNYLTQKPQDEAKENKL | |
| | KLNFDNPSLAGGWDVNKECSNFCVILKDKNEKKYLAIMKKGENTLF | |
| | QKEWTEGRGKNLTKKSNPLFEINNCEILSKMEYDFWADVSKMIPKCS | |
| | TQLKAVVNHFKQSDNEFIFPIGYKVTSGEKFREECKISKQDFELNNKV | |
| | FNKNELSVTAMRYDLSSTQEKQYIKAFQKEYWELLFKQEKRDTKLT | |
| | NNEIFNEWINFCNKKYSELLSWERKYKDALTNWINFCKYFLSKYPKT | |
| | TLFNYSFKESENYNSLDEFYRDVDICSYKLNINTTINKSILDRLVEEGK | |
| | LYLFEIKNQDSNDGKSIGHKNNLHTIYWNAIFENFDNRPKLNGEAEIF | |
| | YRKAISKDKLGIVKGKKTKNGTEIIKNYRFSKEKFILHVPITLNFCSNN | |
| | EYVNDIVNTKFYNFSNLHFLGIDRGEKHLAYYSLVNKNGEIVDQGTL | |
| | NLPFTDKDGNQRSIKKEKYFYNKQEDKWEAKEVDCWNYNDLLDA | |
| | MASNRDMARKNWQRIGTIKEAKNGYVSLVIRKIADLAVNNERPAFI | |
| | VLEDLNTGFKRSRQKIDKSVYQKFELALAKKLNFLVDKNAKRDEIGS | |
| | PTKALQLTPPVNNYGDIENKKQAGIMLYTRANYTSQTDPATGWRKT | |
| | IYLKAGPEETTYKKDGKIKNKSVKDQIIETFTDIGFDGKDYYFEYDKG | |
| | EFVDEKTGEIKPKKWRLYSGENGKSLDRFRGEREKDKYEWKIDKIDI | |
| | VKILDDLFVNFDKNISLLKQLKEGVELTRNNEHGTGESLRFAINLIQQI | |
| | RNTGNNERDNDFILSPVRDENGKHFDSREYWDKETKGEKISMPSSGD | |
| | ANGAFNIARKGIIMNAHILANSDSKDLSLFVSDEEWDLHLNNKTEWK | |
| | KQLNIFSSRKAMAKRKK | |
| PdCpf1 | MENYQEFTNLFQLNKTLRFELKPIGKTCELLEEGKIFASGSFLEKDKV | SEQ |
| | RADNVSYVKKEIDKKHKIFIEETLSSFSISNDLLKQYFDCYNELKAFK | ID |
| | KDCKSDEEEVKKTALRNKCTSIQRAMREAISQAFLKSPQKKLLAIKN | NO: |
| | LIENVFKADENVQHFSEFTSYFSGFETNRENFYSDEEKSTSIAYRLVH | 770 |
| | DNLPIFIKNIYIFEKLKEQFDAKTLSEIFENYKLYVAGSSLDEVFSLEYF | |
| | NNTLTQKGIDNYNAVIGKIVKEDKQEIQGLNEHINLYNQKHKDRRLP | |
| | FFISLKKQILSDREALSWLPDMFKNDSEVIKALKGFYIEDGFENNVLT | |
| | PLATLLSSLDKYNLNGIFIRNNEALSSLSQNVYRNFSIDEAIDANAELQ | |
| | TFNNYELIANALRAKIKKETKQGRKSFEKYEEYIDKKVKAIDSLSIQEI | |
| | NELVENYVSEFNSNSGNMPRKVEDYFSLMRKGDFGSNDLIENIKTKL | |
| | SAAEKLLGTKYQETAKDIFKKDENSKLIKELLDATKQFQHFIKPLLGT | |
| | GEEADRDLVFYGDFLPLYEKFEELTLLYNKVRNRLTQKPYSKDKIRL | |
| | CFNKPKLMTGWVDSKTEKSDNGTQYGGYLFRKKNEIGEYDYFLGIS | |
| | SKAQLFRKNEAVIGDYERLDYYQPKANTIYGSAYEGENSYKEDKKR | |
| | LNKVIIAYIEQIKQTNIKKSIIESISKYPNISDDDKVTPSSLLEKIKKVSID | |
| | SYNGILSFKSFQSVNKEVIDNLLKTISPLKNKAEFLDLINKDYQIFTEV | |
| | QAVIDEICKQKTFIYFPISNVELEKEMGDKDKPLCLFQISNKDLSFAKT | |
| | FSANLRKKRGAENLHTMLFKALMEGNQDNLDLGSGAIFYRAKSLDG | |
| | NKPTHPANEAIKCRNVANKDKVSLFTYDIYKNRRYMENKFLFHLSIV | |
| | QNYKAANDSAQLNSSATEYIRKADDLHIIGIDRGERNLLYYSVIDMK | |
| | GNIVEQDSLNIIRNNDLETDYHDLLDKREKERKANRQNWEAVEGIK | |
| | DLKKGYLSQAVHQIAQLMLKYNAIIALEDLGQMFVTRGQKIEKAVY | |
| | QQFEKSLVDKLSYLVDKKRPYNELGGILKAYQLASSITKNNSDKQNG | |
| | FLFYVPAWNTSKIDPVTGFTDLLRPKAMTIKEAQDFFGAFDNISYND | |
| | KGYFEFETNYDKFKIRMKSAQTRWTICTFGNRIKRKKDKNYWNYEE | |
| | VELTEEFKKLFKDSNIDYENCNLKEEIQNKDNRKFFDDLIKLLQLTLQ | |
| | MRNSDDKGNDYIISPVANAEGQFFDSRNGDKKLPLDADANGAYNIA | |
| | RKGLWNIRQIKQTKNDKKLNLSISSTEWLDFVREKPYLK | |
| MbCpf1 | MLFQDFTHLYPLSKTVRFELKPIDRTLEHIHAKNFLSQDETMADMHQ | SEQ |
| | KVKVILDDYHRDFIADMMGEVKLTKLAEFYDVYLKFRKNPKDDEL | ID |
| | QKQLKDLQAVLRKEIVKPIGNGGKYKAGYDRLFGAKLFKDGKELGD | NO: |
| | LAKFVIAQEGESSPKLAHLAHFEKFSTYFTGFHDNRKNMYSDEDKHT | 771 |
| | AIAYRLIHENLPRFIDNLQILTTIKQKHSALYDQIINELTASGLDVSLAS | |
| | HLDGYHKLLTQEGITAYNTLLGGISGEAGSPKIQGINELINSHHNQHC | |
| | HKSERIAKLRPLHKQILSDGMSVSFLPSKFADDSEMCQAVNEFYRHY | |
| | ADVFAKVQSLFDGFDDHQKDGIYVEHKNLNELSKQAFGDFALLGRV | |
| | LDGYYVDVVNPEFNERFAKAKTDNAKAKLTKEKDKFIKGVHSLASL | |
| | EQAIEHYTARHDDESVQAGKLGQYFKHGLAGVDNPIQKIHNNHSTIK | |
| | GFLERERPAGERALPKIKSGKNPEMTQLRQLKELLDNALNVAHFAKL | |
| | LTTKTTLDNQDGNFYGEFGVLYDELAKIPTLYNKVRDYLSQKPFSTE | |
| | KYKLNFGNPTLLNGWDLNKEKDNFGVILQKDGCYYLALLDKAHKK | |
| | VFDNAPNTGKSIYQKMIYKYLEVRKQFPKVFFSKEAIAINYHPSKELV | |
| | EIKDKGRQRSDDERLKLYRFILECLKIHPKYDKKFEGAIGDIQLFKKD | |
| | KKGREVPISEKDLFDKINGIFSSKPKLEMEDFFIGEFKRYNPSQDLVD | |
| | QYNIYKKIDSNDNRKKENFYNNHPKFKKDLVRYYYESMCKHEEWE | |
| | ESFEFSKKLQDIGCYVDVNELFTEIETRRLNYKISFCNINADYIDELVE | |
| | QGQLYLFQIYNKDFSPKAHGKPNLHTLYFKALFSEDNLADPIYKLNG | |
| | EAQIFYRKASLDMNETTIHRAGEVLENKNPDNPKKRQFVYDIIKDKR | |
| | YTQDKFMLHVPITMNFGVQGMTIKEFNKKVNQSIQQYDEVNVIGIDR | |
| | GERHLLYLTVINSKGEILEQCSLNDITTASANGTQMTTPYHKILDKRE | |
| | IERLNARVGWGEIETIKELKSGYLSHVVHQISQLMLKYNAIVVLEDL | |
| | NFGFKRGRFKVEKQIYQNFENALIKKLNHLVLKDKADDEIGSYKNAL | |
| | QLTNNFTDLKSIGKQTGFLFYVPAWNTSKIDPETGFVDLLKPRYENIA | |
| | QSQAFFGKFDKICYNADKDYFEFHIDYAKFTDKAKNSRQIWTICSHG | |
| | DKRYVYDKTANQNKGAAKGINVNDELKSLFARHHINEKQPNLVMDI | |
| | CQNNDKEFHKSLMYLLKTLLALRYSNASSDEDFILSPVANDEGVFFN | |
| | SALADDTQPQNADANGAYHIALKGLWLLNELKNSDDLNKVKLAID | |
| | NQTWLNFAQNR | |
| EeCpf1 | MNGNRSIVYREFVGVIPVAKTLRNELRPVGHTQEHIIQNGLIQEDELR | SEQ |
| | QEKSTELKNIMDDYYREYIDKSLSGVTDLDFTLLFELMNLVQSSPSK | ID |
| | DNKKALEKEQSKMREQICTHLQSDSNYKNIFNAKLLKEILPDFIKNY | NO: |
| | NQYDVKDKAGKLETLALFNGFSTYFTDFFEKRKNVFTKEAVSTSIAY | 772 |
| | RIVHENSLIFLANMTSYKKISEKALDEIEVIEKNNQDKMGDWELNQIF | |
| | NPDFYNMVLIQSGIDFYNEICGVVNAHMNLYCQQTKNNYNLFKMR | |
| | KLHKQILAYTSTSFEVPKMFEDDMSVYNAVNAFIDETEKGNIIGKLK | |
| | DIVNKYDELDEKRIYISKDFYETLSCFMSGNWNLITGCVENFYDENIH | |
| | AKGKSKEEKVKKAVKEDKYKSINDVNDLVEKYIDEKERNEFKNSNA | |
| | KQYIREISNIITDTETAHLEYDDHISLIESEEKADEMKKRLDMYMNMY | |
| | HWAKAFIVDEVLDRDEMFYSDIDDIYNILENIVPLYNRVRNYVTQKP | |
| | YNSKKIKLNFQSPTLANGWSQSKEFDNNAIILIRDNKYYLAIFNAKNK | |
| | PDKKIIQGNSDKKNDNDYKKMVYNLLPGANKMLPKVFLSKKGIETF | |
| | KPSDYIISGYNAHKHIKTSENFDISFCRDLIDYFKNSIEKHAEWRKYEF | |
| | KFSATDSYSDISEFYREVEMQGYRIDWTYISEADINKLDEEGKIYLFQI | |
| | YNKDFAENSTGKENLHTMYFKNIFSEENLKDIIIKLNGQAELFYRRAS | |
| | VKNPVKHKKDSVLVNKTYKNQLDNGDVVRIPIPDDIYNEIYKMYNG | |
| | YIKESDLSEAAKEYLDKVEVRTAQKDIVKDYRYTVDKYFIHTPITINY | |
| | KVTARNNVNDMVVKYIAQNDDIHVIGIDRGERNLIYISVIDSHGNIVK | |
| | QKSYNILNNYDYKKKLVEKEKTREYARKNWKSIGNIKELKEGYISG | |
| | VVHEIAMLIVEYNAIIAMEDLNYGFKRGRFKVERQVYQKFESMLINK | |
| | LNYFASKEKSVDEPGGLLKGYQLTYVPDNIKNLGKQCGVIFYVPAAF | |
| | TSKIDPSTGFISAFNFKSISTNASRKQFFMQFDEIRYCAEKDMFSFGFD | |
| | YNNFDTYNITMGKTQWTVYTNGERLQSEFNNARRTGKTKSINLTETI | |
| | KLLLEDNEINYADGHDIRIDMEKMDEDKKSEFFAQLLSLYKLTVQM | |
| | RNSYTEAEEQENGISYDKIISPVINDEGEFFDSDNYKESDDKECKMPK | |
| | DADANGAYCIALKGLYEVLKIKSEWTEDGFDRNCLKLPHAEWLDFI | |
| | QNKRYE | |
| CmtCpf1 | MNNYDEFTKLYPIQKTIRFELKPQGRTMEHLETFNFFEEDRDRAEKY | SEQ |
| | KILKEAIDEYHKKFIDEHLTNMSLDWNSLKQISEKYYKSREEKDKKV | ID |
| | FLSEQKRMRQEIVSEFKKDDRFKDLFSKKLFSELLKEEIYKKGNHQEI | NO: |
| | DALKSFDKFSGYFIGLHENRKNMYSDGDEITAISNRIVNENFPKFLDN | 773 |
| | LQKYQEARKKYPEWIIKAESALVAHNIKMDEVFSLEYFNKVLNQEGI | |
| | QRYNLALGGYVTKSGEKMMGLNDALNLAHQSEKSSKGRIHMTPLF | |
| | KQILSEKESFSYIPDVFTEDSQLLPSIGGFFAQIENDKDGNIFDRALELI | |
| | SSYAEYDTERIYIRQADINRVSNVIFGEWGTLGGLMREYKADSINDIN | |
| | LERTCKKVDKWLDSKEFALSDVLEAIKRTGNNDAFNEYISKMRTAR | |
| | EKIDAARKEMKFISEKISGDEESIHIIKTLLDSVQQFLHFFNLFKARQDI | |
| | PLDGAFYAEFDEVHSKLFAIVPLYNKVRNYLTKNNLNTKKIKLNFKN | |
| | PTLANGWDQNKVYDYASLIFLRDGNYYLGIINPKRKKNIKFEQGSGN | |
| | GPFYRKMVYKQIPGPNKNLPRVFLTSTKGKKEYKPSKEIIEGYEADK | |
| | HIRGDKFDLDFCHKLIDFFKESIEKHKDWSKFNFYFSPTESYGDISEFY | |
| | LDVEKQGYRMHFENISAETIDEYVEKGDLFLFQIYNKDFVKAATGKK | |
| | DMHTIYWNAAFSPENLQDVVVKLNGEAELFYRDKSDIKEIVHREGEI | |
| | LVNRTYNGRTPVPDKIHKKLTDYHNGRTKDLGEAKEYLDKVRYFK | |
| | AHYDITKDRRYLNDKIYFHVPLTLNFKANGKKNLNKMVIEKFLSDE | |
| | KAHIIGIDRGERNLLYYSIIDRSGKIIDQQSLNVIDGFDYREKLNQREIE | |
| | MKDARQSWNAIGKIKDLKEGYLSKAVHEITKMAIQYNAIVVMEELN | |
| | YGFKRGRFKVEKQIYQKFENMLIDKMNYLVFKDAPDESPGGVLNAY | |
| | QLTNPLESFAKLGKQTGILFYVPAAYTSKIDPTTGFVNLFNTSSKTNA | |
| | QERKEFLQKFESISYSAKDGGIFAFAFDYRKFGTSKTDHKNVWTAYT | |
| | NGERMRYIKEKKRNELFDPSKEIKEALTSSGIKYDGGQNILPDILRSN | |
| | NNGLIYTMYSSFIAAIQMRVYDGKEDYIISPIKNSKGEFFRTDPKRREL | |
| | PIDADANGAYNIALRGELTMRAIAEKFDPDSEKMAKLELKHKDWFE | |
| | FMQTRGD | |
| BsCpf1 | MYYQNLTKKYPVSKTIRNELIPIGKTLENIRKNNILESDVKRKQDYEH | SEQ |
| | VKGIMDEYHKQLINEALDNYMLPSLNQAAEIYLKKHVDVEDREEFK | ID |
| | KTQDLLRREVTGRLKEHENYTKIGKKDILDLLEKLPSISEEDYNALES | NO: |
| | FRNFYTYFTSYNKVRENLYSDEEKSSTVAYRLINENLPKFLDNIKSYA | 774 |
| | FVKAAGVLADCIEEEEQDALFMVETFNMTLTQEGIDMYNYQIGKVN | |
| | SAINLYNQKNHKVEEFKKIPKMKVLYKQILSDREEVFIGEFKDDETLL | |
| | SSIGAYGNVLMTYLKSEKINIFFDALRESEGKNVYVKNDLSKTTMSNI | |
| | VFGSWSAFDELLNQEYDLANENKKKDDKYFEKRQKELKKNKSYTL | |
| | EQMSNLSKEDISPIENYIERISEDIEKICIYNGEFEKIVVNEHDSSRKLS | |
| | KNIKAVKVIKDYLDSIKELEHDIKLINGSGQELEKNLVVYVGQEEALE | |
| | QLRPVDSLYNLTRNYLTKKPFSTEKVKLNFNKSTLLNGWDKNKETD | |
| | NLGILFFKDGKYYLGIMNTTANKAFVNPPAAKTENVFKKVDYKLLP | |
| | GSNKMLPKVFFAKSNIGYYNPSTELYSNYKKGTHKKGPSFSIDDCHN | |
| | LIDFFKESIKKHEDWSKFGFEFSDTADYRDISEFYREVEKQGYKLTFT | |
| | DIDESYINDLIEKNELYLFQIYNKDFSEYSKGKLNLHTLYFMMLFDQR | |
| | NLDNVVYKLNGEAEVFYRPASIAENELVIHKAGEGIKNKNPNRAKV | |
| | KETSTFSYDIVKDKRYSKYKFTLHIPITMNFGVDEVRRENDVINNALR | |
| | TDDNVNVIGIDRGERNLLYVVVINSEGKILEQISLNSIINKEYDIETNY | |
| | HALLDEREDDRNKARKDWNTIENIKELKTGYLSQVVNVVAKLVLK | |
| | YNAIICLEDLNFGFKRGRQKVEKQVYQKFEKMLIEKLNYLVIDKSRE | |
| | QVSPEKMGGALNALQLTSKFKSFAELGKQSGIIYYVPAYLTSKIDPTT | |
| | GFVNLFYIKYENIEKAKQFFDGFDFIRFNKKDDMFEFSFDYKSFTQKA | |
| | CGIRSKWIVYTNGERIIKYPNPEKNNLFDEKVINVTDEIKGLFKQYRIP | |
| | YENGEDIKEIIISKAEADFYKRLFRLLHQTLQMRNSTSDGTRDYIISPV | |
| | KNDRGEFFCSEFSEGTMPKDADANGAYNIARKGLWVLEQIRQKDEG | |
| | EKVNLSMTNAEWLKYAQLHLL | |
| BhCas12b | MGIHGVPAAATRSFILKIEPNEEVKKGLWKTHEVLNHGIAYYMNILK | SEQ |
| | LIRQEAIYEHHEQDPKNPKKVSKAEIQAELWDFVLKMQKCNSFTHEV | ID |
| | DKDEVFNILRELYEELVPSSVEKKGEANQLSNKFLYPLVDPNSQSGK | NO: |
| | GTASSGRKPRWYNLKIAGDPSWEEEKKKWEEDKKKDPLAKILGKLA | 775 |
| | EYGLIPLFIPYTDSNEPIVKEIKWMEKSRNQSVRRLDKDMFIQALERF | |
| | LSWESWNLKVKEEYEKVEKEYKTLEERIKEDIQALKALEQYEKERQ | |
| | EQLLRDTLNTNEYRLSKRGLRGWREIIQKWLKMDENEPSEKYLEVF | |
| | KDYQRKHPREAGDYSVYEFLSKKENHFIWRNHPEYPYLYATFCEIDK | |
| | KKKDAKQQATFTLADPINHPLWVRFEERSGSNLNKYRILTEQLHTEK | |
| | LKKKLTVQLDRLIYPTESGGWEEKGKVDIVLLPSRQFYNQIFLDIEEK | |
| | GKHAFTYKDESIKFPLKGTLGGARVQFDRDHLRRYPHKVESGNVGRI | |
| | YFNMTVNIEPTESPVSKSLKIHRDDFPKVVNFKPKELTEWIKDSKGK | |
| | KLKSGIESLEIGLRVMSIDLGQRQAAAASIFEVVDQKPDIEGKLFFPIK | |
| | GTELYAVHRASFNIKLPGETLVKSREVLRKAREDNLKLMNQKLNFL | |
| | RNVLHFQQFEDITEREKRVTKWISRQENSDVPLVYQDELIQIRELMY | |
| | KPYKDWVAFLKQLHKRLEVEIGKEVKHWRKSLSDGRKGLYGISLKN | |
| | IDEIDRTRKFLLRWSLRPTEPGEVRRLEPGQRFAIDQLNHLNALKEDR | |
| | LKKMANTIIMHALGYCYDVRKKKWQAKNPACQIILFEDLSNYNPYG | |
| | ERSRFENSRLMKWSRREIPRQVALQGEIYGLQVGEVGAQFSSRFHAK | |
| | TGSPGIRCRVVTKEKLQDNRFFKNLQREGRLTLDKIAVLKEGDLYPD | |
| | KGGEKFISLSKDRKCVTTHADINAAQNLQKRFWTRTHGFYKVYCKA | |
| | YQVDGQTVYIPESKDQKQKIIEEFGEGYFILKDGVYEWVNAGKLKIK | |
| | KGSSKQSSSELVDSDILKDSFDLASELKGEKLMLYRDPSGNVFPSDK | |
| | WMAAGVFFGKLERILISKLTNQYSISTIEDDSSKQS | |
| AkCas12b | MAVKSIKVKLRLSECPDILAGMWQLHRATNAGVRYYTEWVSLMRQ | SEQ |
| | EILYSRGPDGGQQCYMTAEDCQRELLRRLRNRQLHNGRQDQPGTDA | ID |
| | DLLAISRRLYEILVLQSIGKRGDAQQIASSFLSPLVDPNSKGGRGEAKS | NO: |
| | GRKPAWQKMRDQGDPRWVAAREKYEQRKAVDPSKEILNSLDALGL | 776 |
| | RPLFAVFTETYRSGVDWKPLGKSQGVRTWDRDMFQQALERLMSWE | |
| | SWNRRVGEEYARLFQQKMKFEQEHFAEQSHLVKLARALEADMRAA | |
| | SQGFEAKRGTAHQITRRALRGADRVFEIWKSIPEEALFSQYDEVIRQV | |
| | QAEKRRDFGSHDLFAKLAEPKYQPLWRADETFLTRYALYNGVLRDL | |
| | EKARQFATFTLPDACVNPIWTRFESSQGSNLHKYEFLFDHLGPGRHA | |
| | VRFQRLLVVESEGAKERDSVVVPVAPSGQLDKLVLREEEKSSVALHL | |
| | HDTARPDGFMAEWAGAKLQYERSTLARKARRDKQGMRSWRRQPS | |
| | MLMSAAQMLEDAKQAGDVYLNISVRVKSPSEVRGQRRPPYAALFRI | |
| | DDKQRRVTVNYNKLSAYLEEHPDKQIPGAPGLLSGLRVMSVDLGLR | |
| | TSASISVFRVAKKEEVEALGDGRPPHYYPIHGTDDLVAVHERSHLIQ | |
| | MPGETETKQLRKLREERQAVLRPLFAQLALLRLLVRCGAADERIRTR | |
| | SWQRLTKQGREFTKRLTPSWREALELELTRLEAYCGRVPDDEWSRI | |
| | VDRTVIALWRRMGKQVRDWRKQVKSGAKVKVKGYQLDVVGGNSL | |
| | AQIDYLEQQYKFLRRWSFFARASGLVVRADRESHFAVALRQHIENA | |
| | KRDRLKKLADRILMEALGYVYEASGPREGQWTAQHPPCQLIILEELS | |
| | AYRFSDDRPPSENSKLMAWGHRGILEELVNQAQVHDVLVGTVYAA | |
| | FSSRFDARTGAPGVRCRRVPARFVGATVDDSLPLWLTEFLDKHRLD | |
| | KNLLRPDDVIPTGEGEFLVSPCGEEAARVRQVHADINAAQNLQRRL | |
| | WQNFDITELRLRCDVKMGGEGTVLVPRVNNARAKQLFGKKVLVSQ | |
| | DGVTFFERSQTGGKPHSEKQTDLTDKELELIAEADEARAKSVVLFRD | |
| | PSGHIGKGHWIRQREFWSLVKQRIESHTAERIRVRGVGSSLD | |
| BsCas12b | MAIRSIKLKLKTHTGPEAQNLRKGIWRTHRLLNEGVAYYMKMLLLF | SEQ |
| | RQESTGERPKEELQEELICHIREQQQRNQADKNTQALPLDKALEALR | ID |
| | QLYELLVPSSVGQSGDAQIISRKFLSPLVDPNSEGGKGTSKAGAKPT | NO: |
| | WQKKKEANDPTWEQDYEKWKKRREEDPTASVITTLEEYGIRPIFPLY | 777 |
| | TNTVTDIAWLPLQSNQFVRTWDRDMLQQAIERLLSWESWNKRVQE | |
| | EYAKLKEKMAQLNEQLEGGQEWISLLEQYEENRERELRENMTAAN | |
| | DKYRITKRQMKGWNELYELWSTFPASASHEQYKEALKRVQQRLRG | |
| | RFGDAHFFQYLMEEKNRLIWKGNPQRIHYFVARNELTKRLEEAKQS | |
| | ATMTLPNARKHPLWVRFDARGGNLQDYYLTAEADKPRSRRFVTFSQ | |
| | LIWPSESGWMEKKDVEVELALSRQFYQQVKLLKNDKGKQKIEFKDK | |
| | GSGSTFNGHLGGAKLQLERGDLEKEEKNFEDGEIGSVYLNVVIDFEP | |
| | LQEVKNGRVQAPYGQVLQLIRRPNEFPKVTTYKSEQLVEWIKASPQH | |
| | SAGVESLASGFRVMSIDLGLRAAAATSIFSVEESSDKNAADFSYWIEG | |
| | TPLVAVHQRSYMLRLPGEQVEKQVMEKRDERFQLHQRVKFQIRVLA | |
| | QIMRMANKQYGDRWDELDSLKQAVEQKKSPLDQTDRTFWEGIVCD | |
| | LTKVLPRNEADWEQAVVQIHRKAEEYVGKAVQAWRKRFAADERK | |
| | GIAGLSMWNIEELEGLRKLLISWSRRTRNPQEVNRFERGHTSHQRLL | |
| | THIQNVKEDRLKQLSHAIVMTALGYVYDERKQEWCAEYPACQVILF | |
| | ENLSQYRSNLDRSTKENSTLMKWAHRSIPKYVHMQAEPYGIQIGDV | |
| | RAEYSSRFYAKTGTPGIRCKKVRGQDLQGRRFENLQKRLVNEQFLTE | |
| | EQVKQLRPGDIVPDDSGELFMTLTDGSGSKEVVFLQADINAAHNLQ | |
| | KRFWQRYNELFKVSCRVIVRDEEEYLVPKTKSVQAKLGKGLFVKKS | |
| | DTAWKDVYVWDSQAKLKGKTTFTEESESPEQLEDFQEIIEEAEEAKG | |
| | TYRTLFRDPSGVFFPESVWYPQKDFWGEVKRKLYGKLRERFLTKAR | |
| AmCas12b | MNVAVKSIKVKLMLGHLPEIREGLWHLHEAVNLGVRYYTEWLALL | SEQ |
| | RQGNLYRRGKDGAQECYMTAEQCRQELLVRLRDRQKRNGHTGDPG | ID |
| | TDEELLGVARRLYELLVPQSVGKKGQAQMLASGFLSPLADPKSEGG | NO: |
| | KGTSKSGRKPAWMGMKEAGDSRWVEAKARYEANKAKDPTKQVIA | 778 |
| | SLEMYGLRPLFDVFTETYKTIRWMPLGKHQGVRAWDRDMFQQSLE | |
| | RLMSWESWNERVGAEFARLVDRRDRFREKHFTGQEHLVALAQRLE | |
| | QEMKEASPGFESKSSQAHRITKRALRGADGIIDDWLKLSEGEPVDRF | |
| | DEILRKRQAQNPRRFGSHDLFLKLAEPVFQPLWREDPSFLSRWASYN | |
| | EVLNKLEDAKQFATFTLPSPCSNPVWARFENAEGTNIFKYDFLFDHF | |
| | GKGRHGVRFQRMIVMRDGVPTEVEGIVVPIAPSRQLDALAPNDAASP | |
| | IDVFVGDPAAPGAFRGQFGGAKIQYRRSALVRKGRREEKAYLCGFR | |
| | LPSQRRTGTPADDAGEVFLNLSLRVESQSEQAGRRNPPYAAVFHISD | |
| | QTRRVIVRYGEIERYLAEHPDTGIPGSRGLTSGLRVMSVDLGLRTSAA | |
| | ISVFRVAHRDELTPDAHGRQPFFFPIHGMDHLVALHERSHLIRLPGET | |
| | ESKKVRSIREQRLDRLNRLRSQMASLRLLVRTGVLDEQKRDRNWER | |
| | LQSSMERGGERMPSDWWDLFQAQVRYLAQHRDASGEAWGRMVQ | |
| | AAVRTLWRQLAKQVRDWRKEVRRNADKVKIRGIARDVPGGHSLAQ | |
| | LDYLERQYRFLRSWSAFSVQAGQVVRAERDSRFAVALREHIDNGKK | |
| | DRLKKLADRILMEALGYVYVTDGRRAGQWQAVYPPCQLVLLEELS | |
| | EYRFSNDRPPSENSQLMVWSHRGVLEELIHQAQVHDVLVGTIPAAFS | |
| | SRFDARTGAPGIRCRRVPSIPLKDAPSIPIWLSHYLKQTERDAAALRP | |
| | GELIPTGDGEFLVTPAGRGASGVRVVHADINAAHNLQRRLWENFDL | |
| | SDIRVRCDRREGKDGTVVLIPRLTNQRVKERYSGVIFTSEDGVSFTVG | |
| | DAKTRRRSSASQGEGDDLSDEEQELLAEADDARERSVVLFRDPSGFV | |
| | NGGRWTAQRAFWGMVHNRIETLLAERFSVSGAAEKVRG | |
| AaCas12b | MAVKSMKVKLRLDNMPEIRAGLWKLHTEVNAGVRYYTEWLSLLR | SEQ |
| | QENLYRRSPNGDGEQECYKTAEECKAELLERLRARQVENGHCGPAG | ID |
| | SDDELLQLARQLYELLVPQAIGAKGDAQQIARKFLSPLADKDAVGG | NO: |
| | LGIAKAGNKPRWVRMREAGEPGWEEEKAKAEARKSTDRTADVLRA | 779 |
| | LADFGLKPLMRVYTDSDMSSVQWKPLRKGQAVRTWDRDMFQQAIE | |
| | RMMSWESWNQRVGEAYAKLVEQKSRFEQKNFVGQEHLVQLVNQL | |
| | QQDMKEASHGLESKEQTAHYLTGRALRGSDKVFEKWEKLDPDAPF | |
| | DLYDTEIKNVQRRNTRRFGSHDLFAKLAEPKYQALWREDASFLTRY | |
| | AVYNSIVRKLNHAKMFATFTLPDATAHPIWTRFDKLGGNLHQYTFL | |
| | FNEFGEGRHAIRFQKLLTVEDGVAKEVDDVTVPISMSAQLDDLLPRD | |
| | PHELVALYFQDYGAEQHLAGEFGGAKIQYRRDQLNHLHARRGARD | |
| | VYLNLSVRVQSQSEARGERRPPYAAVFRLVGDNHRAFVHFDKLSDY | |
| | LAEHPDDGKLGSEGLLSGLRVMSVDLGLRTSASISVFRVARKDELKP | |
| | NSEGRVPFCFPIEGNENLVAVHERSQLLKLPGETESKDLRAIREERQR | |
| | TLRQLRTQLAYLRLLVRCGSEDVGRRERSWAKLIEQPMDANQMTPD | |
| | WREAFEDELQKLKSLYGICGDREWTEAVYESVRRVWRHMGKQVRD | |
| | WRKDVRSGERPKIRGYQKDVVGGNSIEQIEYLERQYKFLKSWSFFGK | |
| | VSGQVIRAEKGSRFAITLREHIDHAKEDRLKKLADRIIMEALGYVYA | |
| | LDDERGKGKWVAKYPPCQLILLEELSEYQFNNDRPPSENNQLMQWS | |
| | HRGVFQELLNQAQVHDLLVGTMYAAFSSRFDARTGAPGIRCRRVPA | |
| | RCAREQNPEPFPWWLNKFVAEHKLDGCPLRADDLIPTGEGEFFVSPF | |
| | SAEEGDFHQIHADLNAAQNLQRRLWSDFDISQIRLRCDWGEVDGEP | |
| | VLIPRTTGKRTADSYGNKVFYTKTGVTYYERERGKKRRKVFAQEEL | |
| | SEEEAELLVEADEAREKSVVLMRDPSGIINRGDWTRQKEFWSMVNQ | |
| | RIEGYLVKQIRSRVRLQESACENTGDI | |
| RfxCas13d | MIEKKKSFAKGMGVKSTLVSGSKVYMTTFAEGSDARLEKIVEGDSIR | SEQ |
| | SVNEGEAFSAEMADKNAGYKIGNAKFSHPKGYAVVANNPLYTGPV | ID |
| | QQDMLGLKETLEKRYFGESADGNDNICIQVIHNILDIEKILAEYITNA | NO: |
| | AYAVNNISGLDKDIIGFGKFSTVYTYDEFKDPEHHRAAFNNNDKLIN | 780 |
| | AIKAQYDEFDNFLDNPRLGYFGQAFFSKEGRNYIINYGNECYDILALL | |
| | SGLRHWVVHNNEEESRISRTWLYNLDKNLDNEYISTLNYLYDRITNE | |
| | LTNSFSKNSAANVNYIAETLGINPAEFAEQYFRFSIMKEQKNLGFNIT | |
| | KLREVMLDRKDMSEIRKNHKVFDSIRTKVYTMMDFVIYRYYIEEDA | |
| | KVAAANKSLPDNEKSLSEKDIFVINLRGSFNDDQKDALYYDEANRIW | |
| | RKLENIMHNIKEFRGNKTREYKKKDAPRLPRILPAGRDVSAFSKLMY | |
| | ALTMFLDGKEINDLLTTLINKFDNIQSFLKVMPLIGVNAKFVEEYAFF | |
| | KDSAKIADELRLIKSFARMGEPIADARRAMYIDAIRILGTNLSYDELK | |
| | ALADTFSLDENGNKLKKGKHGMRNFIINNVISNKRFHYLIRYGDPAH | |
| | LHEIAKNEAVVKFVLGRIADIQKKQGQNGKNQIDRYYETCIGKDKG | |
| | KSVSEKVDALTKIITGMNYDQFDKKRSVIEDTGRENAEREKFKKIISL | |
| | YLTVIYHILKNIVNINARYVIGFHCVERDAQLYKEKGYDINLKKLEEK | |
| | GFSSVTKLCAGIDETAPDKRKDVEKEMAERAKESIDSLESANPKLYA | |
| | NYIKYSDEKKAEEFTRQINREKAKTALNAYLRNTKWNVIIREDLLRI | |
| | DNKTCTLFRNKAVHLEVARYVHAYINDIAEVNSYFQLYHYIMQRIIM | |
| | NERYEKSSGKVSEYFDAVNDEKKYNDRLLKLLCVPFGYCIPRFKNLS | |
| | IEALFDRNEAAKFDKEKKKVSGN | |
| LwCas13a | MKVTKVDGISHKKYIEEGKLVKSTSEENRTSERLSELLSIRLDIYIKNP | SEQ |
| | DNASEEENRIRRENLKKFFSNKVLHLKDSVLYLKNRKEKNAVQDKN | ID |
| | YSEEDISEYDLKNKNSFSVLKKILLNEDVNSEELEIFRKDVEAKLNKI | NO: |
| | NSLKYSFEENKANYQKINENNVEKVGGKSKRNIIYDYYRESAKRND | 781 |
| | YINNVQEAFDKLYKKEDIEKLFFLIENSKKHEKYKIREYYHKIIGRKN | |
| | DKENFAKIIYEEIQNVNNIKELIEKIPDMSELKKSQVFYKYYLDKEEL | |
| | NDKNIKYAFCHFVEIEMSQLLKNYVYKRLSNISNDKIKRIFEYQNLKK | |
| | LIENKLLNKLDTYVRNCGKYNYYLQVGEIATSDFIARNRQNEAFLRN | |
| | IIGVSSVAYFSLRNILETENENGITGRMRGKTVKNNKGEEKYVSGEV | |
| | DKIYNENKQNEVKENLKMFYSYDFNMDNKNEIEDFFANIDEAISSIR | |
| | HGIVHFNLELEGKDIFAFKNIAPSEISKKMFQNEINEKKLKLKIFKQLN | |
| | SANVFNYYEKDVIIKYLKNTKFNFVNKNIPFVPSFTKLYNKIEDLRNT | |
| | LKFFWSVPKDKEEKDAQIYLLKNIYYGEFLNKFVKNSKVFFKITNEVI | |
| | KINKQRNQKTGHYKYQKFENIEKTVPVEYLAIIQSREMINNQDKEEK | |
| | NTYIDFIQQIFLKGFIDYLNKNNLKYIESNNNNDNNDIFSKIKIKKDNK | |
| | EKYDKILKNYEKHNRNKEIPHEINEFVREIKLGKILKYTENLNMFYLI | |
| | LKLLNHKELTNLKGSLEKYQSANKEETFSDELELINLLNLDNNRVTE | |
| | DFELEANEIGKFLDFNENKIKDRKELKKFDTNKIYFDGENIIKHRAFY | |
| | NIKKYGMLNLLEKIADKAKYKISLKELKEYSNKKNEIEKNYTMQQN | |
| | LHRKYARPKKDEKFNDEDYKEYEKAIGNIQKYTHLKNKVEFNELNL | |
| | LQGLLLKILHRLVGYTSIWERDLRFRLKGEFPENHYIEEIFNFDNSKN | |
| | VKYKSGQIVEKYINFYKELYKDNVEKRSIYSDKKVKKLKQEKKDLYI | |
| | RNYIAHFNYIPHAEISLLEVLENLRKLLSYDRKLKNAIMKSIVDILKEY | |
| | GFVATFKIGADKKIEIQTLESEKIVHLKNLKKKKLMTDRNSEELCELV | |
| | KVMFEYKALE | |
| PspCas13b | MNIPALVENQKKYFGTYSVMAMLNAQTVLDHIQKVADIEGEQNEN | SEQ |
| | NENLWFHPVMSHLYNAKNGYDKQPEKTMFIIERLQSYFPFLKIMAEN | ID |
| | QREYSNGKYKQNRVEVNSNDIFEVLKRAFGVLKMYRDLTNHYKTY | NO: |
| | EEKLNDGCEFLTSTEQPLSGMINNYYTVALRNMNERYGYKTEDLAFI | 782 |
| | QDKRFKFVKDAYGKKKSQVNTGFFLSLQDYNGDTQKKLHLSGVGIA | |
| | LLICLFLDKQYINIFLSRLPIFSSYNAQSEERRIIIRSFGINSIKLPKDRIHS | |
| | EKSNKSVAMDMLNEVKRCPDELFTTLSAEKQSRFRIISDDHNEVLMK | |
| | RSSDRFVPLLLQYIDYGKLFDHIRFHVNMGKLRYLLKADKTCIDGQT | |
| | RVRVIEQPLNGFGRLEEAETMRKQENGTFGNSGIRIRDFENMKRDDA | |
| | NPANYPYIVDTYTHYILENNKVEMFINDKEDSAPLLPVIEDDRYVVK | |
| | TIPSCRMSTLEIPAMAFHMFLFGSKKTEKLIVDVHNRYKRLFQAMQK | |
| | EEVTAENIASFGIAESDLPQKILDLISGNAHGKDVDAFIRLTVDDMLT | |
| | DTERRIKRFKDDRKSIRSADNKMGKRGFKQISTGKLADFLAKDIVLF | |
| | QPSVNDGENKITGLNYRIMQSAIAVYDSGDDYEAKQQFKLMFEKAR | |
| | LIGKGTTEPHPFLYKVFARSIPANAVEFYERYLIERKFYLTGLSNEIKK | |
| | GNRVDVPFIRRDQNKWKTPAMKTLGRIYSEDLPVELPRQMFDNEIKS | |
| | HLKSLPQMEGIDFNNANVTYLIAEYMKRVLDDDFQTFYQWNRNYR | |
| | YMDMLKGEYDRKGSLQHCFTSVEEREGLWKERASRTERYRKQASN | |
| | KIRSNRQMRNASSEEIETILDKRLSNSRNEYQKSEKVIRRYRVQDALL | |
| | FLLAKKTLTELADFDGERFKLKEIMPDAEKGILSEIMPMSFTFEKGGK | |
| | KYTITSEGMKLKNYGDFFVLASDKRIGNLLELVGSDIVSKEDIMEEFN | |
| | KYDQCRPEISSIVFNLEKWAFDTYPELSARVDREEKVDFKSILKILLN | |
| | NKNINKEQSDILRKIRNAFDHNNYPDKGVVEIKALPEIAMSIKKAFGE | |
| | YAIMKGSLQ | |
| PguCas13b | MTEQSERPYNGTYYTLEDKHFWAAFLNLARHNAYITLTHIDRQLAY | SEQ |
| | SKADITNDQDVLSFKALWKNFDNDLERKSRLRSLILKHFSFLEGAAY | ID |
| | GKKLFESKSSGNKSSKNKELTKKEKEELQANALSLDNLKSILFDFLQ | NO: |
| | KLKDFRNYYSHYRHSGSSELPLFDGNMLQRLYNVFDVSVQRVKIDH | 783 |
| | EHNDEVDPHYHFNHLVRKGKKDRYGHNDNPSFKHHFVDGEGMVTE | |
| | AGLLFFVSLFLEKRDAIWMQKKIRGFKGGTETYQQMTNEVFCRSRIS | |
| | LPKLKLESLRMDDWMLLDMLNELVRCPKPLYDRLREDDRACFRVP | |
| | VDILPDEDDTDGGGEDPFKNTLVRHQDRFPYFALRYFDLKKVFTSLR | |
| | FHIDLGTYHFAIYKKMIGEQPEDRHLTRNLYGFGRIQDFAEEHRPEE | |
| | WKRLVRDLDYFETGDKPYISQTSPHYHIEKGKIGLRFMPEGQHLWPS | |
| | PEVGTTRTGRSKYAQDKRLTAEAFLSVHELMPMMFYYFLLREKYSE | |
| | EVSAERVQGRIKRVIEDVYAVYDAFARDEINTRDELDACLADKGIRR | |
| | GHLPRQMIAILSQEHKDMEEKIRKKLQEMMADTDHRLDMLDRQTD | |
| | RKIRIGRKNAGLPKSGVIADWLVRDMMRFQPVAKDASGKPLNNSKA | |
| | NSTEYRMLQRALALFGGEKERLTPYFRQMNLTGGNNPHPFLHETRW | |
| | ESHTNILSFYRSYLRARKAFLERIGRSDRVENRPFLLLKEPKTDRQTL | |
| | VAGWKGEFHLPRGIFTEAVRDCLIEMGHDEVASYKEVGFMAKAVPL | |
| | YFERACEDRVQPFYDSPFNVGNSLKPKKGRFLSKEERAEEWERGKE | |
| | RFRDLEAWSYSAARRIEDAFAGIEYASPGNKKKIEQLLRDLSLWEAF | |
| | ESKLKVRADRINLAKLKKEILEAQEHPYHDFKSWQKFERELRLVKN | |
| | QDIITWMMCRDLMEENKVEGLDTGTLYLKDIRPNVQEQGSLNVLNR | |
| | VKPMRLPVVVYRADSRGHVHKEEAPLATVYIEERDTKLLKQGNFKS | |
| | FVKDRRLNGLFSFVDTGGLAMEQYPISKLRVEYELAKYQTARVCVF | |
| | ELTLRLEESLLTRYPHLPDESFREMLESWSDPLLAKWPELHGKVRLLI | |
| | AVRNAFSHNQYPMYDEAVESSIRKYDPSSPDAIEERMGLNIAHRLSE | |
| | EVKQAKETVERIIQAGSLQ | |
| RanCas13b | MEKPLLPNVYTLKHKFFWGAFLNIARHNAFITICHINEQLGLKTPSND | SEQ |
| | DKIVDVVCETWNNILNNDHDLLKKSQLTELILKHFPFLTAMCYHPPK | ID |
| | KEGKKKGHQKEQQKEKESEAQSQAEALNPSKLIEALEILVNQLHSLR | NO: |
| | NYYSHYKHKKPDAEKDIFKHLYKAFDASLRMVKEDYKAHFTVNLT | 784 |
| | RDFAHLNRKGKNKQDNPDFNRYRFEKDGFFTESGLLFFTNLFLDKR | |
| | DAYWMLKKVSGFKASHKQREKMTTEVFCRSRILLPKLRLESRYDHN | |
| | QMLLDMLSELSRCPKLLYEKLSEENKKHFQVEADGFLDEIEEEQNPF | |
| | KDTLIRHQDRFPYFALRYLDLNESFKSIRFQVDLGTYHYCIYDKKIGD | |
| | EQEKRHLTRTLLSFGRLQDFTEINRPQEWKALTKDLDYKETSNQPFIS | |
| | KTTPHYHITDNKIGFRLGTSKELYPSLEIKDGANRIAKYPYNSGFVAH | |
| | AFISVHELLPLMFYQHLTGKSEDLLKETVRHIQRIYKDFEEERINTIED | |
| | LEKANQGRLPLGAFPKQMLGLLQNKQPDLSEKAKIKIEKLIAETKLLS | |
| | HRLNTKLKSSPKLGKRREKLIKTGVLADWLVKDFMRFQPVAYDAQ | |
| | NQPIKSSKANSTEFWFIRRALALYGGEKNRLEGYFKQTNLIGNTNPHP | |
| | FLNKFNWKACRNLVDFYQQYLEQREKFLEAIKNQPWEPYQYCLLLK | |
| | IPKENRKNLVKGWEQGGISLPRGLFTEAIRETLSEDLMLSKPIRKEIKK | |
| | HGRVGFISRAITLYFKEKYQDKHQSFYNLSYKLEAKAPLLKREEHYE | |
| | YWQQNKPQSPTESQRLELHTSDRWKDYLLYKRWQHLEKKLRLYRN | |
| | QDVMLWLMTLELTKNHFKELNLNYHQLKLENLAVNVQEADAKLNP | |
| | LNQTLPMVLPVKVYPATAFGEVQYHKTPIRTVYIREEHTKALKMGN | |
| | FKALVKDRRLNGLFSFIKEENDTQKHPISQLRLRRELEIYQSLRVDAF | |
| | KETLSLEEKLLNKHTSLSSLENEFRALLEEWKKEYAASSMVTDEHIA | |
| | FIASVRNAFCHNQYPFYKEALHAPIPLFTVAQPTTEEKDGLGIAEALL | |
| | KVLREYCEIVKSQIGSSLQKKLEELELGSS | |

In some embodiments, the Cas protein is a Cas9, a dead Cas9 (dCas9), or a Cas9 nickase (nCas9).

In some embodiments, the Cas protein is a nCas9. In some embodiments, the nCas9 protein is a nCas9-D10A protein. In some embodiments, the nCas9-D10A protein has an amino acid sequence of SEQ ID NO: 146.

In some embodiments, the first protein-binding RNA motif and the first RNA binding domain, the second protein-binding RNA motif and the second RNA binding domain, and the third protein-binding RNA motif and the third RNA binding domain, are each independently selected from the group consisting of a MS2 phage operator stem-loop and MS2 coat protein (MCP) or an RNA-binding section thereof; a BoxB and N22P or an RNA-binding section thereof; a telomerase Ku binding motif and Ku protein or an RNA-binding section thereof; a telomerase Sm7 binding motif and Sm7 protein or an RNA-binding section thereof; a PP7 phage operator stem-loop and PP7 coat protein (PCP) or an RNA-binding section thereof; a SfMu phage Coin stem-loop and Coin RNA binding protein or an RNA-binding section thereof; and a non-natural RNA aptamer and corresponding aptamer ligand or an RNA-binding section thereof. See Table 11.

TABLE 11

| | | SEQ ID |
|---|---|---|
| Name | Sequence | NO |
| MS2 | ACAUGAGGAUCACCCAUGU | SEQ ID |
| | | NO: 244 |
| PP7 | GGAGCAGACGAUAUGGCGUCGCUCC | SEQ ID |
| | | NO: 245 |
| boxB | GCCCUGAAGAAGGGC | SEQ ID |
| | | NO: 246 |
| mgRNA | GUUUGAGAGCUAGGCCAACAUGAGGAUCACCCAU | SEQ ID |
| scaffold with | GUCUGCAGGGCCUAGCAAGUUCAAAUAAGGCUAG | NO: 147 |
| 1xMS2 | UCCGUUAUCAACUUGAAAAAGUGGCACCGAGUCG | |
| | GUGC | |
| mgRNA | GUUUGAGAGCUAGGCCAACAUGAGGAUCACCCAU | SEQ ID |
| scaffold with | GUCUGCAGGGCCUAGCAAGUUCAAAUAAGGCUAG | NO: 148 |
| 2xMS2 | UCCGUUAUCAACUUGGCCAACAUGAGGAUCACCC | |
| | AUGUCUGCAGGGCCAAGUGGCACCGAGUCGGUGC | |
| mgRNA | GUUUGAGAGCUACCGGAGCAGACGAUAUGGCGU | SEQ ID |
| scaffold with | CGCUCCGGUAGCAAGUUCAAAUAAGGCUAGUCCG | NO: 149 |
| 1xPP7 | UUAUCAACUUGAAAAAGUGGCACCGAGUCGGUGC | |
| mgRNA | GUUUGAGAGCUACCGGAGCAGACGAUAUGGCGU | SEQ ID |
| scaffold with | CGCUCCGGUAGCAAGUUCAAAUAAGGCUAGUCCG | NO: 150 |
| 2xPP7 | UUAUCAACUUGGAGCAGACGAUAUGGCGUCGCU | |
| | CCAAGUGGCACCGAGUCGGUGC | |
| mgRNA | GUUUGAGAGCUAGGGCCCUGAAGAAGGGCCCUA | SEQ ID |
| scaffold with | GCAAGUUCAAAUAAGGCUAGUCCGUUAUCAACUU | NO: 151 |
| 1xboxB | GAAAAAGUGGCACCGAGUCGGUGC | |
| mgRNA | GUUUGAGAGCUAGGGCCCUGAAGAAGGGCCCUA | SEQ ID |
| scaffold with | GCAAGUUCAAAUAAGGCUAGUCCGUUAUCAACUU | NO: 152 |
| 2xboxB | GGGCCCUGAAGAAGGGCCCAAGUGGCACCGAGUC | |
| | GGUGC | |
| MS2 coat | MASNFTQFVLVDNGGTGDVTVAPSNFANGIAEWISSN | SEQ ID |
| protein | SRSQAYKVTCSVRQSSAQNRKYTIKVEVPKGAWRSY | NO: 153 |
| (MSP) | LNMELTIPIFATNSDCELIVKAMQGLLKDGNPIPSAIAA | |
| | NMGIY | |
| PP7 coat | MGSKTIVLSVGEATRTLTEIQSTADRQIFEEKVGPLVG | SEQ ID |
| protein | RLRLTASLRQNGAKTAYRVNLKLDQADVVDMGLPK | NO: 154 |
| (PCP) | VRYTQVWSHDVTIVANSTEASRKSLYDLTKSLVATSQ | |
| | VEDLVVNLVPLGR | |
| boxB coat | MGNARTRRRERRAEKQAQWKAAN | SEQ ID |
| protein | | NO: 155 |
| (N22p) | | |
| Telomerase | TTGTGTTTCTACTTATAGATGGCTAAAATCTGAGTT | SEQ ID |
| Ku binding | TAGAAAATGCAA | NO: 785 |
| motif | | |
| Telomerase | AATTTTTGGA | SEQ ID |
| Sm7 binding | | NO: 786 |
| motif | | |
| SfMu | CTGAATGCCTGCGAGCATC | SEQ ID |
| | | NO: 787 |
| YKU70 | MELDPDDVFRDEDEDPENDFFQEKEASKEFVVYLIDA | SEQ ID |
| | SPKMFCSTCPSEEEDKQESHFHIAVSCIAQSLKAHIINR | NO: 788 |
| | SNDEIAICFFNTREKKNLQDLNGVYVFNVPERDSIDRP | |
| | TARLIKEFDLIEESFDKEIGSQTGIVSDSRENSLYSALW | |
| | VAQALLRKGSLKTADKRMFLFTNEDDPFGSMRISVKE | |
| | DMTRTTLQRAKDAQDLGISIELLPLSQPDKQFNITLFY | |
| | KDLIGLNSDELTEFMPSVGQKLEDMKDQLKKRVLAK | |
| | RIAKRITFVICDGLSIELNGYALLRPAIPGSITWLDSTTN | |
| | LPVKVERSYICTDTGAIMQDPIQRIQPYKNQNIMFTVE | |
| | ELSQVKRISTGHLRLLGFKPLSCLKDYHNLKPSTFLYP | |
| | SDKEVIGSTRAFIALHRSMIQLERFAVAFYGGTTPPRL | |
| | VALVAQDEIESDGGQVEPPGINMIYLPYANDIRDIDEL | |
| | HSKPGVAAPRASDDQLKKASALMRRLELKDFSVCQF | |
| | ANPALQRHYAILQAIALDENELRETRDETLPDEEGMN | |
| | RPAVVKAIEQFKQSIYGDDPDEESDSGAKEKSKKRKA | |
| | GDADDGKYDYIELAKTGKLKDLTVVELKTYLTANNL | |
| | LVSGKKEVLINRILTHIGK | |
| YKU80 | MSSESTTFIVDVSPSMMKNNNVSKSMAYLEYTLINKS | SEQ ID |
| | KKSRKTDWISCYLANCPVSENSQEIPNVFQIQSFLAPV | NO: 789 |
| | TTTATIGFIKRLKQYCDQHSHDSSNEGLQSMIQCLLVV | |
| | SLDIKQQFQARKILKQIVVFTDNLDDLDITDEEIDLLTE | |
| | ELSTRIILIDCGKDTQEERKKSNWLKLVEAIPNSRIYN | |
| | MNELLVEITSPATSVVKPVRVFSGELRLGADILSTQTS | |
| | NPSGSMQDENCLCIKVEAFPATKAVSGLNRKTAVEVE | |
| | DSQKKERYVGVKSIIEYEIHNEGNKKNVSEDDQSGSS | |
| | YIPVTISKDSVTKAYRYGADYVVLPSVLVDQTVYESF | |
| | PGLDLRGFLNREALPRYFLTSESSFITADTRLGCQSDL | |
| | MAFSALVDVMLENRKIAVARYVSKKDSEVNMCALCP | |
| | VLIEHSNINSEKKFVKSLTLCRLPFAEDERVTDFPKLLD | |
| | RTTTSGVPLKKETDGHQIDELMEQFVDSMDTDELPEIP | |
| | LGNYYQPIGEVTTDTTLPLPSLNKDQEENKKDPLRIPT | |
| | VFVYRQQQVLLEWIHQLMINDSREFEIPELPDSLKNKI | |
| | SPYTHKKFDSTKLVEVLGIKKVDKLKLDSELKTELER | |
| | EKIPDLETLLKRGEQHSRGSPNNSNN | |
| Sm7-like | GSVIDVSSQRVNVQRPLDALGNSLNSPVIIKLKGDREF | SEQ ID |
| protein | RGVLKSFDLHMNLVLNDAEELEDGEVTRRLGT | NO: 790 |
| SfMu Com | MKSIRCKNCNKLLFKADSFDHIEIRCPRCKRHIIMLNA | SEQ ID |
| binding | CEHPTEKHCGKREKITHSDETVRY | NO: 791 |
| protein | | |

For any protein of the present disclosure, biological equivalents thereof are also provided. In some embodiments, the biological equivalents have at least about 70%, 75%, 80%, 85%, 90%, 95%, 98%, or 99% sequence identity with the reference protein. Preferably, the biological equivalents retain the desired activity of the reference protein. In some embodiments, the biological equivalents are derived by including one, two, three, four, five, or more amino acid additions, deletions, substitutions, or the combinations thereof. In some embodiments, the substitution is a conservative amino acid substitution.

In another aspect, the present disclosure provides a kit comprising one or more gene editing system targeting the PDCD1 gene, the TRAC gene, the B2M gene, the CD52 gene, the CTLA4 gene, the TIGIT gene, the TIM3 gene, the LAG3 gene, the CISH gene, the TGFBR2 gene, the FAS gene, the CD7 gene, the CBLB gene, the KLRC1 gene, and/or the CD38 gene.

In some embodiments, the present disclosure provides a kit comprising a first gene editing system targeting the PDCD1 gene, and a second gene editing system targeting the TRAC gene, the B2M gene, the CD52 gene, the CTLA4 gene, the TIGIT gene, the TIM3 gene, the LAG3 gene, the CISH gene, the TGFBR2 gene, the FAS gene, the CD7 gene, the CBLB gene, the KLRC11 gene, and/or the CD38 gene.

In some embodiments, the present disclosure provides a kit comprising a first gene editing system targeting the TRAC gene, and a second gene editing system targeting a gene selected from CD352, B2M, PDCD1, CTLA4, TIGIT, TIM3, LAG3, CISH, TGFBR2, FAS, CD7, CBLB, and CD38.

In some embodiments, the present disclosure provides a kit comprising a first gene editing system targeting the CD52 gene, and a second gene editing system targeting agene selected from PDCD1, CTLA4, TIGIT, TIM3, LAG3, CISH, TGFBR2, FAS, CD7, CBLB, KLRC1, and CD38.

In some embodiments, the present disclosure provides a kit comprising a first gene editing system targeting the B2M gene, and a second gene editing system targeting a gene selected from PDCD1, CTLA4, TIGIT, TIM3, LAG3, CISH, TGFBR2, FAS, CD7, CBLB, KLRC1, and CD38.

In some embodiments, the present disclosure provides a kit comprising a first gene editing system targeting the PDCD1 gene, and a second gene editing system targeting a gene selected from CTLA4, TIGIT, TIM3, LAG3, CISH, TGFBR2, FAS, CD7, CBLB, KLRC1, and CD38.

In some embodiments, the present disclosure provides a kit comprising a first gene editing system targeting the CTLA4 gene, and a second gene editing system targeting a gene selected from TIGIT, TIM3, LAG3, CISH, TGFBR2, FAS, CD7, CBLB, KLRC1, and CD38.

In some embodiments, the present disclosure provides a kit comprising a first gene editing system targeting the TIGIT gene, and a second gene editing system targeting a gene selected from TIM3, LAG3, CISH, TGFBR2, FAS, CD7, CBLB, KLRC1, and CD38.

In some embodiments, the present disclosure provides a kit comprising a first gene editing system targeting the TIM3 gene, and a second gene editing system targeting a gene selected from LAG3, CISH, TGFBR2, FAS, CD7, CBLB, KLRC1, and CD38.

In some embodiments, the present disclosure provides a kit comprising a first gene editing system targeting the LAG3 gene, and a second gene editing system targeting a gene selected from CISH, TGFBR2, FAS, CD7, CBLB, KLRC1, and CD38.

In some embodiments, the present disclosure provides a kit comprising a first gene editing system targeting the CISH gene, and a second gene editing system targeting a gene selected from TGFBR2, FAS, CD7, CBLB, KLRC1, and CD38.

In some embodiments, the present disclosure provides a kit comprising a first gene editing system targeting the TGFBR2 gene, and a second gene editing system targeting a gene selected from FAS, CD7, CBLB, KLRC1, and CD38.

In some embodiments, the present disclosure provides a kit comprising a first gene editing system targeting the FAS gene, and a second gene editing system targeting a gene selected from CD7, CBLB, KLRC1, and CD38.

In some embodiments, the present disclosure provides a kit comprising a first gene editing system targeting the CD7 gene, and a second gene editing system targeting a gene selected from CBLB and KLRC1.

In some embodiments, the present disclosure provides a kit comprising a first gene editing system targeting the CBLB gene, and a second gene editing system targeting a gene as selected from KLRC1 and CD38.

In some embodiments, the present disclosure provides a kit comprising a first gene editing system targeting the KLRC1 gene, and a second gene editing system targeting CD38.

In some embodiments, the present disclosure provides a kit comprising a first gene editing system targeting the KLRC1 gene, and a second gene editing system targeting CD38.

In some embodiments, the present disclosure provides a kit comprising a first gene editing system targeting the KLRC1 gene, and a second gene editing system targeting a gene selected from PD1, TGFBR2, CISH, CD38, CBLB, TIGIT, TIM-3, LAG3, FAS, and TGFBR2.

In some embodiments of the gene editing systems described herein, the guide RNA (the (main) single guide RNA and/or the helper guide RNA) is a dual-RNA structure formed by a ligand-bound CRISPR RNA (crRNA) and a trans-activating crRNA (tracrRNA). In some embodiments, the crRNA comprises a spacer sequence and is capable of forming a base-pair structure with the tracrRNA, and wherein the base-pair structure binds to a Cas protein. In some embodiments, the crRNA further comprises a linker sequence which comprises a protein-binding motif. For the purpose of the present disclosure, when the guide RNA is a dual-RNA structure of crRNA and tracrRNA, the “CRISPR motif” refers to the base-pair structure formed between the crRNA and the tracrRNA.

In some embodiments, the gene editing system is a LIGO-RNA-based gene editing system, as described in PCT/CN2023/096482, which is incorporated herein by reference in its entirety. A person skilled in the art would be able to design the corresponding crRNA-tracrRNA pair based on the sgRNA and hsgRNA disclosed herein.

In the LigoRNA-based gene editing system, at least one guide RNA is a LigoRNA. A LigoRNA system comprises a dual-RNA structure, which can be used as a guide RNA in CRISPR-based gene editing systems. The dual-RNA structure can be formed by a ligand-bound CRISPR RNA (crRNA) and a trans-activating crRNA (tracrRNA). For example, the LigoRNA system comprises an hgRNA set of a hcrRNA and a tracrRNA, and an mgRNA set of mcrRNA and a tracrRNA. Preferably, all of these RNA molecules are not longer than 100 nucleotides.

Since the LigoRNA system is formed by two short RNAs, it helps to solve the problem of synthesizing long single guide RNAs in previous gene editing systems. Chemically synthesized RNAs over 100 nt demonstrated much lower yield and purity, resulting in challenges for large-scale production and cost control.

Original types of crRNA and tracrRNA are capable of guiding nCas9-mediated DNA location. The crRNAs and the tracrRNAs in the LigoRNA system are further modified. In some embodiments, an MS2 or boxB hairpin is fused to crRNA in multiple different sites. In some embodiments, at least one nucleotide in the crRNAs and the tracrRNAs is modified, such as by a 2′-O-methyl modification and/or 3′-phosphorothioate modification.

In some embodiments, the crRNA comprises a spacer sequence and a linker sequence, as wherein the linker sequence comprises at least one protein-binding motif, wherein the protein-binding motif is an RNA aptamer motif. In some embodiments, the protein binding motif is selected from MS2, PP7, boxB, SfMu hairpin motif, telomerase Ku, and Sm7 binding motif, or a variant thereof. Aptamers are single-stranded oligonucleotides that fold into defined architectures and selectively bind to a specific target, including proteins, peptides, carbohydrates, small molecules, toxins, and even live cells.

In some embodiments, the crRNA is capable of forming a base-pair structure with a trans-activating crRNA (tracrRNA). In some embodiments, the tracrRNA has an sequence of SEQ ID NO: 804 or 811.

In some embodiments, the crRNA comprises at least one nucleotide with modification. In some embodiments, the modification is selected from 2′-O-alkyl, 2′-substituted alkoxy, 2′-substituted alkyl, 2′-halo, 3′-phosphorothioate, bridged nucleic acid (BNA), and locked nucleic acid (LNA). In some embodiments, the at least one nucleotide with modification is any one of the first three nucleotides from 3′-end of the engineered crRNA.

In some embodiments, the tracrRNA comprises at least one nucleotide with modification. In some embodiments, the modification is selected from 2′-O-alkyl, 2′-substituted alkoxy, 2′-substituted alkyl, 2′-halo, 3′-phosphorothioate, bridged nucleic acid (BNA), and locked nucleic acid (LNA). In some embodiments, the at least one nucleotide with modification is any one of the first three nucleotides from 3′-end of the engineered tracrRNA.

In some embodiments, the crRNA and/or tracrRNA comprises at least one nucleotide with modification. In some embodiments, the modification is selected from 2′-O-alkyl (such as 2′-O-methyl), 2′-substituted alkoxy, 2′-substituted alkyl, 2′-halo (such as 2′-fluoro), 3′-phosphorothioate, bridged nucleic acid (BNA), and locked nucleic acid (LNA). In some embodiments, the crRNA and/or tracrRNA comprises nucleotides comprising 2′-O-methyl and 3′-phosphorothioate. In some embodiments, the first three nucleotides from the 5′-end of the crRNA and/or tracrRNA are modified with 2′-O-methyl and 3′-phosphorothioate. In some embodiments, the first three nucleotides from the 3′-end of the crRNA and/or tracrRNA are modified with 2′-O-methyl, and the second to fourth nucleotides from the 3′-end of the crRNA and/or tracrRNA are modified with 3′-phosphorothioate. In some embodiments, the first three nucleotides from the 5′-end of the crRNA and/or tracrRNA are modified with 2′-O-methyl and 3′-phosphorothioate, and the first three nucleotides from the 3′-end of the crRNA and/or tracrRNA are modified with 2′-O-methyl, and the second to fourth nucleotides from the 3′-end of the crRNA and/or tracrRNA are modified with 3′-phosphorothioate.

In some embodiments, is the present disclosure provides a tBE system comprising two LigoRNA structures: an mcrRNA-tracrRNA base-paired structure and an hcrRNA-tracrRNA base-paired structure. In some embodiments, the mcrRNA contains a boxB hairpin to generate an R-loop region for intended base editing and the hcrRNA contains an MS2 hairpin to recruit a nucleotide deaminase (e.g., an APOBEC linked to a nucleobase deaminase inhibitor (e.g., a cytosine deaminase inhibitor (dCDI)) domain through a cleavage site such as a TEV protease cleavage site. For example, to cleave off the dCDI domain at the on-target sites, an N22p-fused as TEVc is recruited by the boxB-containing mcrRNA, working as the key in tBE system with free TEVn. In some embodiments, mcrRNA and hcrRNA form a base-paired structure with the same tracrRNA to locate a target DNA, and the dCDI domain is cleaved off at the target site to induce efficient base editing.

In some embodiments of the gene editing system described herein, the gene editing system comprises
a. an hcrRNA comprising a first spacer sequence and a first linker sequence, wherein the first linker sequence comprises a first protein-binding motif,
b. an mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
wherein the first Cas protein and the second Cas protein are the same or different, and the first tracrRNA and the second tracrRNA are the same or different.

In some embodiments of the gene editing system described herein, the gene editing system further comprises
a. a protease, or a polynucleotide encoding the protease, and
b. a nucleobase deaminase inhibitor domain,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof.

In some embodiments of the gene editing system described herein, the gene editing system comprises
a. an hcrRNA comprising a first spacer sequence and a first linker sequence, wherein the first linker sequence comprises a first protein-binding motif,
b. an mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease,
i. a nucleobase deaminase inhibitor domain, and
j. a second fusion protein comprising the protease and a second RNA binding domain, or a polynucleotide encoding the second fusion protein,
wherein the first Cas protein and the second Cas protein are the same or different, and the first tracrRNA and the second tracrRNA are the same or different, wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the protease and the second RNA binding domain are optionally connected by a linker, and
wherein the second RNA binding domain binds to the second protein-binding motif.

In some embodiments of the gene editing system described herein, the protease is split into a first protease fragment and a second protease fragment, wherein the first and/or second protease fragment alone is not able to cleave the cleavage site.

In some embodiments of the gene editing system described herein, wherein the gene editing system comprises
a. an hcrRNA comprising a first spacer sequence and a first linker sequence, wherein the first linker sequence comprises a first protein-binding motif,
b. an mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease, wherein the protease is split into a first protease fragment and a second protease fragment, wherein the first and/or second protease fragment alone is not able to cleave the cleavage site,
i. a nucleobase deaminase inhibitor domain,
j. a second fusion protein comprising the first protease fragment and a second RNA binding domain, or a polynucleotide encoding the second fusion protein, wherein the first protease fragment and the second RNA binding domain are optionally connected by a linker, and
k. a third fusion protein comprising the second protease fragment and a third RNA binding domain, or a polynucleotide encoding the third fusion protein, wherein the second protease fragment and the third RNA binding domain are optionally connected by a linker,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the mcrRNA further comprises a third protein-binding motif,
wherein the second RNA binding domain binds to the second protein-binding motif, and
wherein the third RNA binding domain binds to the third protein-binding motif.

In some embodiments of the gene editing system described herein, the gene editing system comprises
a. an hcrRNA comprising a first spacer sequence and a first linker sequence, wherein the first linker sequence comprises a first protein-binding motif,
b. an mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease, wherein the protease is split into a first protease fragment and a second protease fragment, wherein the first and/or second protease fragment alone is not able to cleave the cleavage site,
i. a nucleobase deaminase inhibitor domain,
j. a second fusion protein comprising the first protease fragment and a second RNA binding domain, or a polynucleotide encoding the second fusion protein, wherein the first protease fragment and the second RNA binding domain are optionally connected by a linker, and
k. a third fusion protein comprising the second protease fragment and a third RNA binding domain, or a polynucleotide encoding the third fusion protein, wherein the second protease fragment and the third RNA binding domain are optionally connected by a linker,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the mcrRNA further comprises a third protein-binding motif,
wherein the second RNA binding domain binds to the second protein-binding motif,
wherein the third RNA binding domain binds to the third protein-binding motif, and
wherein the second and the third RNA binding domains are the same or different, and the second and the third protein-binding motifs are the same or different.

In some embodiments of the gene editing system described herein, the gene editing system comprises
a. an hcrRNA comprising a first spacer sequence and a first linker sequence, wherein the first linker sequence comprises a first protein-binding motif,
b. an mcrRNA comprising a second spacer sequence and a second linker sequence, wherein the second linker sequence comprises a second protein-binding motif,
c. a first tracrRNA which is capable of forming a first base-pair structure with the hcrRNA,
d. a second tracrRNA which is capable of forming a second base-pair structure with the mcrRNA,
e. a first CRISPR-associated protein (Cas protein), or a polynucleotide encoding the first Cas protein, wherein the first Cas protein binds to the first base-pair structure,
f. a second Cas protein, or a polynucleotide encoding the second Cas protein, wherein the second Cas protein binds to the second base pair structure,
g. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
h. a protease, or a polynucleotide encoding the protease, wherein the protease is split into a first protease fragment and a second protease fragment, wherein the first and/or second protease fragment alone is not able to cleave the cleavage site,
i. a nucleobase deaminase inhibitor domain,
j. a second fusion protein comprising the first protease fragment and a second RNA binding domain, or a polynucleotide encoding the second fusion protein,
wherein the first Cas protein and the second Cas protein are the same or different, and the first tracrRNA and the second tracrRNA are the same or different,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof,
wherein the first protease fragment and the second RNA binding domain are optionally connected by a linker, and
wherein the second RNA binding domain binds to the second protein-binding motif.

### Polynucleotides

In another aspect, the present disclosure provides a polynucleotide encoding the hgRNA and/or the mgRNA disclosed in at least one of the gene editing systems herein.

In another aspect, the present disclosure provides a polynucleotide encoding all components except the first and the second Cas protein in the gene editing system disclosed herein.

In another aspect, the present disclosure provides a polynucleotide encoding all components in the gene editing system disclosed herein.

In another aspect, the present disclosure provides a kit comprising a polynucleotide encoding all components except the first and the second Cas protein in the gene editing system disclosed herein, and a polynucleotide encoding the first and/or second Cas protein in the gene editing system disclosed herein. In some embodiments, the first and the second Cas proteins are the same Cas protein.

The polynucleotides disclosed herein can be obtained by methods known in the art. For example, the polynucleotide can be obtained from cloned DNA (e.g., from a DNA library), by chemical synthesis, by cDNA cloning, or by the cloning of genomic DNA or fragments thereof, purified from the desired cell. When the polynucleotides are produced by recombinant means, any method known to those skilled in the art for identification of nucleic acids that encode desired genes can be used. Any method available in the art can be used to obtain a full length (i.e., encompassing the entire coding region) cDNA or genomic DNA encoding a desired protein, such as from a cell or tissue source. Modified or variant polynucleotides can be engineered from a wildtype polynucleotide using standard recombinant DNA methods. Polynucleotides can be cloned or isolated using any available methods known in the art for cloning and isolating nucleic acid molecules. Such methods include PCR amplification of nucleic acids and screening of libraries, including nucleic acid hybridization screening, antibody-based screening, and activity-based screening.

Methods for amplification of polynucleotides can be used to isolate polynucleotides encoding a desired protein, including for example, polymerase chain reaction (PCR) methods. PCR can be carried out using any known methods or procedures in the art. Exemplary methods include use of a Perkin-Elmer Cetus thermal cycler and Taq polymerase (Gene Amp). A nucleic acid containing gene of interest can be used as a source material from which a desired polypeptide-encoding nucleic acid molecule can be amplified. For example, DNA and mRNA preparations, cell extracts, tissue extracts from an appropriate source (e.g., testis, prostate, breast), fluid samples (e.g., blood, serum, saliva), samples from healthy and/or diseased subjects can be used in amplification methods. The source can be from any eukaryotic species including, but not limited to, vertebrate, mammalian, human, porcine, bovine, feline, avian, equine, canine, and other primate sources. Nucleic acid libraries also can be used as a source material. Primers can be designed to amplify a desired polynucleotide. For example, primers can be designed based on expressed sequences from which a desired polynucleotide is generated. Primers can be designed based on back-translation of a polypeptide amino acid sequence. If desired, degenerate primers can be used for amplification. Oligonucleotide primers that hybridize to sequences at the 3′ and 5′ termini of the desired sequence can be uses as primers to amplify by PCR from a nucleic acid sample. Primers as can be used to amplify the entire full-length polynucleotide, or a truncated sequence thereof. Nucleic acid molecules generated by amplification can be sequenced and confirmed to encode a desired polypeptide.

### Vectors

In another aspect, the present disclosure provides a vector comprising the polynucleotide encoding the hgRNA and/or the mgRNA disclosed herein.

In another aspect, the present disclosure provides a vector comprising the polynucleotide disclosed herein.

In another aspect, the present disclosure provides a vector comprising the polynucleotide encoding all components except the first and the second Cas protein in the gene editing system disclosed herein.

In another aspect, the present disclosure provides a vector comprising the polynucleotide encoding all components in the gene editing system disclosed herein.

In some embodiments, the vector is a plasmid or a viral vector.

In some embodiments, the vector is a polycistronic vector.

In another aspect, the present disclosure provides a kit comprising the vector disclosed above, and a vector comprising the polynucleotide encoding the first and/or second Cas protein in the gene editing system disclosed herein.

Any methods known in the art for the insertion of DNA fragments into a vector can be used to construct expression vectors comprising a polynucleotide disclosed herein. These methods can include in vitro recombinant DNA and synthetic techniques and in vivo (genetic) recombination. The polynucleotide disclosed herein can be operably linked to control sequences in the expression vector(s) to ensure protein expression. Such control sequences may include, but are not limited to, leader or signal sequences, promoters (e.g., naturally associated or heterologous promoters), ribosomal binding sites, enhancer or activator elements, translational start and termination sequences, and transcription start and termination sequences, and are chosen to be compatible with the host cell chosen to express the proteins. Constitutive or inducible promoters as known in the art are also contemplated. The promoters may be either naturally occurring promoters, hybrid promoters that combine elements of more than one promoter, or synthetic promoters. An expression construct may be present in a cell on an episome, such as a plasmid, or the expression construct may be inserted in a chromosome such as in a gene locus. In some embodiment, the expression vector includes a selectable marker gene to allow the selection of transformed host cells. In some embodiments, the vector is an expression vector comprising a nucleotide sequence encoding a variant polypeptide operably linked to at least one regulatory control sequence. Regulatory control sequence for use herein include promoters, enhancers, and other expression control elements. In some embodiments, the expression vector is designed for the choice of the host cell to be transformed, the particular variant polypeptide desired to be expressed, the vector's copy number, the ability to control that copy number, and/or the expression of any other protein encoded by the vector, such as antibiotic markers.

The vector can include, but is not limited to, viral vectors and plasmid DNA. Viral as vectors can include, but are not limited to, adenoviral vectors, lentiviral vectors, retroviral vectors, and adeno-associated viral vectors. Commonly, expression vectors contain selection markers such as ampicillin-resistance, hygromycin-resistance, tetracycline resistance, kanamycin resistance, or neomycin resistance to permit detection of those cells transformed with the desired DNA sequences. Suitable vectors, promoter, and enhancer elements are known in the art; many are commercially available for generating subject recombinant constructs. In some embodiments, the vector is a polycistronic vector. In some embodiments, the vector is a bicistronic vector or a tricistronic vector. Bicistronic or polycistronic expression vectors may include (1) multiple promoters fused to each of the open reading frames; (2) insertion of splicing signals between genes; (3) fusion of genes whose expressions are driven by a single promoter; and (4) insertion of proteolytic cleavage sites between genes (self-cleavage peptide) or insertion of internal ribosomal entry sites (IRESs) between genes.

A polycistronic vector is used to co-express multiple genes in the same cell. Two strategies are most commonly used to construct a multicistronic vector. First, an Internal Ribosome Entry Site (IRES) element is typically used for bi-cistronic vectors. The IRES element, acting as another ribosome recruitment site, allows initiation of translation from an internal region of the mRNA. Thus, two proteins are translated from one mRNA. IRES elements are quite large (usually 500-600 bp) (Pelletier et al., 1988; Jang et al., 1988). The engineered CD47 proteins disclosed herein have a smaller size compared to the wild-type full-length human CD47, and thus could be used with IRES element in a multicistronic vectors having limited packaging capacity.

In another aspect, the present disclosure provides a vector comprising the polynucleotide encoding the hgRNA and/or the mgRNA disclosed herein.

### Cells

In another aspect, the present disclosure provides a cell comprising the gene editing system disclosed herein.

In another aspect, the present disclosure provides a cell comprising the polynucleotide disclosed herein. In some embodiments, the cell further comprises a polynucleotide encoding the first and/or second Cas protein in the gene editing system disclosed herein.

In another aspect, the present disclosure provides a cell comprising the vector disclosed herein. In some embodiments, the cell further comprises a vector comprising a polynucleotide encoding the first and/or second Cas protein in the gene editing system disclosed herein.

In another aspect, the present disclosure provides a cell comprising the kit disclosed herein.

In some embodiments, the cell is a stem cell.

In some embodiments, the cell is a pluripotent stem cell. Pluripotent stem cells are cells that have the capacity to self-renew by dividing and to develop into the three primary germ cell layers of the early embryo and therefore into all cells of the adult body, but not extra-embryonic tissues such as the placenta. Embryonic stem cells and induced pluripotent stem cells are pluripotent stem cells.

In some embodiments, the cell is an embryonic stem cell (ESC). Embryonic stem cells as are pluripotent stem cells derived from the inner cell mass of a blastocyst, an early-stage pre-implantation embryo.

In some embodiments, the cell is an induced pluripotent stem cell (iPSC). iPSCs are derived from adult somatic cells that have been genetically reprogrammed back into an embryonic-like pluripotent state that enables the development of an unlimited source of any type of cell needed for therapeutic purposes.

“Pluripotent stem cells” as used herein have the potential to differentiate into any of the three germ layers: endoderm (e.g., the stomach lining, gastrointestinal tract, lungs, etc.), mesoderm (e.g., muscle, bone, blood, urogenital tissue, etc.) or ectoderm (e.g., epidermal tissues and nervous system tissues). The term “pluripotent stem cells,” as used herein, also encompasses induced pluripotent stem cells (iPSCs or iPS cells), or a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) As used herein, “hiPSCs” are human induced pluripotent stem cells. In some embodiments, “pluripotent stem cells,” as used herein, also encompasses mesenchymal stem cells (MSCs), and/or embryonic stem cells (ESCs).

In some embodiments, the cell is a hematopoietic stem cell. Hematopoietic stem cells are multipotent primitive cells that can develop into all types of blood cells, including myeloid-lineage and lymphoid-lineage cells. The lymphoid branch includes T cells, B cells, and natural killer (NK) cells. HSCs can be found in several organs, such as peripheral blood, bone marrow, and umbilical cord blood. (Lee J Y, Hong S H. Hematopoietic Stem Cells and Their Roles in Tissue Regeneration. Int J Stem Cells. 2020; 13(1):1-12. doi:10.15283/ijsc19127).

In some embodiments, the cell is an immune cell. Immune cell refers to cells that are involved in the function of the immune system, including both the innate immune system and the adaptive immune system. In some embodiments, the immune cell is selected from the group consisting of T cell, B cell, natural killer cell (NK cell), macrophage, dendritic cell, monocyte, granulocyte, and mast cell.

Cellular immunotherapy, also known as adoptive cell therapy, is an innovative treatment approach that aims to harness body's immune system to eliminate cancer. For better recognition and killing of tumour cells, immune cells including T cells, NK cells, γδT cells, natural killer T (NKT) cells, and even macrophages, can be engineered to express antigen-specific T cell as receptors (TCRs) or chimeric antigen receptors (CARs). (Xie, Guozhu, et al. “CAR-NK cells: A promising cellular immunotherapy for cancer.” EBioMedicine 59 (2020): 102975; Liu, Enli, et al. “Use of CAR-transduced natural killer cells in CD19-positive lymphoid tumors.” New England Journal of Medicine 382.6 (2020): 545-553) Chimeric antigen receptors (CARs, also known as chimeric immunoreceptors, chimeric T cell receptors or artificial T cell receptors) are receptor proteins that have been engineered to give T cells the ability to target a specific protein.

In some embodiments, the cell is an immune cell comprising a chimeric antigen receptor (CAR). In some embodiments, the cell is an immune cell comprising a chimeric antigen receptor (CAR), wherein the immune cell is a T cell, a NK cell, a γδT cell, a NKT cell, or a macrophage.

In some embodiments, the cell is a T cell. In some embodiments, the T cell comprises a chimeric antigen receptor (CAR). In some embodiments, the T cell is a CAR-T cell.

A T cell is a type of lymphocyte. T cells are one of the white blood cells of the immune system and play a central role in the adaptive immune response. CAR-T cells are T cells that have been genetically engineered to produce an artificial chimeric antigen receptor. CAR-T cells can be both CD4+ and CD8+, with a 1-to-1 ratio of both cell types providing synergistic antitumor effects. CAR-T cells can be derived from T cells in a patient's own blood (autologous) or derived from the T cells of another healthy donor (allogeneic). T cells can be obtained from a number of sources including, but not limited to, peripheral blood mononuclear cells, bone marrow, lymph nodes tissue, cord blood, thymus issue, tissue from a site of infection, ascites, pleural effusion, spleen tissue, and tumors. In certain embodiments, T cells can be obtained from a unit of blood collected from a subject using any number of techniques known to the skilled person, such as sedimentation, e.g., FICOLL™ separation, antibody-conjugated bead-based methods such as MACS™ separation (Miltenyi).

In some embodiments, the cell is a NK cell. In some embodiments, the NK cell comprises a chimeric antigen receptor (CAR). In some embodiments, the NK cell is a CAR-NK cell.

NK cells, also known as large granular lymphocytes, are a type of cytotoxic lymphocyte. The role of NK cells is analogous to that of cytotoxic T cells in the vertebrate adaptive immune response. CAR-NK cells are NK cells that have been genetically engineered to produce an artificial chimeric antigen receptor. Compared to the CAR-T cells, CAR-NK cell infusions have reduced risk for GvHD. In some embodiments, besides killing tumour target cells in the CAR-dependent manner, CAR-NK cells can potentially eliminate cancer cells in a CAR-independent manner. CAR-NK cells still possess their natural cytotoxic activity against tumour cells. (Oei, Vincent Yi Sheng, et al. “Intrinsic Functional Potential of NK-Cell Subsets Constrains Retargeting Driven by Chimeric Antigen Receptors Intrinsic Functionality of NK Cells Affects CAR Retargeting.” Cancer immunology research 6.4 (2018): 467-480.)

In some embodiments, the cell is a primary cell. Primary cells are isolated directly from human or animal tissue using enzymatic or mechanical methods. Once isolated, they are placed in an artificial environment in plastic or glass containers supported with specialized medium as containing essential nutrients and growth factors to support proliferation. Primary cells could be of two types: adherent or suspension. Adherent cells require attachment for growth and are said to be anchorage-dependent cells. Adherent cells are usually derived from tissues of organs. Suspension cells do not require attachment for growth and are said to be anchorage-independent cells. Most suspension cells are isolated from the blood system, but some tissue-derived cells can also be used in suspension, such as hepatocytes or intestinal cells. Although primary cells usually have a limited lifespan, they offer a number of advantages compared to cell lines. Primary cell culture enables researchers to study donors and not just cells. Several factors such as age, medical history, race, and sex can be considered when building an experimental model. With a growing trend towards personalized medicine, such donor variability and tissue complexity can be achieved with use of primary cells, but are difficult to replicate with cell lines that are more systematic and uniform in nature and do not capture the true diversity of a living tissue.

In some embodiments, the cell is a differentiated cell. Differentiated cells are cells that have undergone differentiation. They are mature cells that perform a specialized function. Some examples of differentiated cells are epithelial cells, skin fibroblasts, endothelial cells lining the blood vessels, smooth muscle cells, liver cells, nerve cells, human cardiac muscle cells, etc. Generally, these cells have a unique morphology, metabolic activity, membrane potential, and responsiveness to signals facilitating their function in a body tissue or organ.

In some embodiments, the differentiated cell is differentiated from a pluripotent stem cell. In some embodiments, the differentiated cell is differentiated from an iPSC or an ESC.

### Composition

In another aspect, the present disclosure provides a composition comprising the gene editing system disclosed herein.

In another aspect, the present disclosure provides a composition comprising the cell disclosed herein.

As used herein, the term “composition” includes, but is not limited to, a pharmaceutical composition. A “pharmaceutical composition” refers to an active pharmaceutical agent formulated in pharmaceutically acceptable or physiologically acceptable solutions for administration to a cell or an animal, either alone, or in combination with one or more other modalities of therapy. It will also be understood that, if desired, the compositions of the disclosure may be administered in combination with other agents, such as, e.g., cytokines, growth factors, hormones, small molecules, chemotherapeutics, pro-drugs, drugs, antibodies, or other various pharmaceutically active agents. There is virtually no limit to other components that may also be included in the compositions, provided that the additional agents do not adversely affect the ability of the composition to deliver the intended therapy. The phrase “pharmaceutically acceptable” is used herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The compositions may also comprise a pharmaceutically acceptable carrier, diluent, or as excipient. As used herein “pharmaceutically acceptable carrier, diluent, or excipient” includes, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, surfactant, or emulsifier which has been approved by the United States Food and Drug Administration as being acceptable for use in humans or domestic animals. Exemplary pharmaceutically acceptable carriers include, but are not limited to, to sugars, such as lactose, glucose, and sucrose; starches, such as corn starch and potato starch; cellulose, and its derivatives, such as sodium carboxymethyl cellulose, ethyl cellulose, and cellulose acetate; tragacanth; malt; gelatin; talc; cocoa butter; waxes; animal and vegetable fats; paraffins; silicones; bentonites; silicic acid; zinc oxide; oils, such as peanut oil, cottonseed oil, safflower oil, sesame oil, olive oil, corn oil, and soybean oil; glycols, such as propylene glycol; polyols, such as glycerin, sorbitol, mannitol, and polyethylene glycol; esters, such as ethyl oleate, and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and any other compatible substances employed in pharmaceutical formulations.

The liquid pharmaceutical compositions, whether they be solutions, suspensions or other like form, may include one or more of the following: sterile diluents such as water for injection, saline solution, preferably physiological saline; Ringers solution; isotonic sodium chloride; fixed oils such as synthetic mono or diglycerides which may serve as the solvent or suspending medium; polyethylene glycols; glycerin; propylene glycol or other solvents; antibacterial agents, such as benzyl alcohol or methyl paraben; antioxidants such as ascorbic acid or sodium bisulfite; chelating agents, such as ethylenediaminetetraacetic acid; buffers such as acetates, citrates, or phosphates; and agents for the adjustment of tonicity, such as sodium chloride or dextrose. The parenteral preparation can be enclosed in ampoules, disposable syringes, or multiple dose vials made of glass or plastic. An injectable pharmaceutical composition is preferably sterile.

The composition may be suitably developed for intravenous, intratumoral, oral, rectal, vaginal, parenteral, topical, pulmonary, intranasal, buccal, ophthalmic, or another route of administration.

### Methods of Treatment

In another aspect, the present disclosure provides a method for reducing immunogenicity of a cell comprising introducing into the cell any one or more of the gene editing systems disclosed herein. By reducing immunogenicity of cells administered to a subject, their expansion and persistence after administration can be enhanced.

In some embodiments, the cell is an allogenic cell. In some embodiments, the cell is an immune cell. In some embodiments, the immune cell is a T cell, B cell, natural killer cell (NK cell), macrophage, dendritic cell, monocyte, granulocyte, or mast cell. In some embodiments, the immune cell comprises a chimeric antigen receptor. In some embodiments, the cell is a T cell. In some embodiments, the T cell comprises a chimeric antigen receptor. In some embodiments, the T cell is a CAR-T cell. In some embodiments, the cell is a NK cell. In some embodiments, the NK as cell comprises a chimeric antigen receptor. In some embodiments, the NK cell is a CAR-NK cell. In some embodiments, the cell is differentiated from a pluripotent stem cell. In some embodiments, the cell is differentiated from an iPSC or an ESC. In some embodiments, the cell is a primary cell.

In another aspect, the present disclosure provides a method for reducing graft versus host (GvH) response involved in administering an allogenic cell into a subject, comprising reducing immunogenicity of the allogenic cell by introducing into the allogenic cell any one or more of the gene editing systems disclosed herein.

In some embodiments, the allogeneic cell is an immune cell. In some embodiments, the immune cell is a T cell, B cell, natural killer cell (NK cell), macrophage, dendritic cell, monocyte, granulocyte, or mast cell. In some embodiments, the immune cell comprises a chimeric antigen receptor. In some embodiments, the allogeneic cell is a T cell. In some embodiments, the T cell comprises a chimeric antigen receptor. In some embodiments, the T cell is a CAR-T cell. In some embodiments, the allogeneic cell is a NK cell. In some embodiments, the NK cell comprises a chimeric antigen receptor. In some embodiments, the NK cell is a CAR-NK cell. In some embodiments, the cell is differentiated from a pluripotent stem cell. In some embodiments, the cell is differentiated from an iPSC or an ESC. In some embodiments, the cell is a primary cell.

The one or more of the gene editing systems that can be used in the methods for reducing immunogenicity and/or reducing graft versus host (GvH) response can be one, two, three, four, five, six, seven, eight, nine, ten, eleven, or twelve gene editing systems selected from the gene systems targeting the PDCD1 gene, the TRAC gene, the B2M gene, the CD52 gene, the CTLA4 gene, the TIGIT gene, the TIM3 gene, the LAG3 gene, the CISH gene, the TGFBR2 gene, the FAS gene, the CD7 gene, the CBLB gene, the KLRC1 gene, and the CD38 gene disclosed herein.

For example, in some embodiments, the present disclosure provides a method for reducing immunogenicity and/or reducing graft versus host (GvH) response comprising introducing into the cell a first gene editing system targeting the PDCD1 gene, and a second gene editing system targeting the TRAC gene, the B2M gene, the CD52 gene, the CTLA4 gene, the TIGIT gene, the TIM3 gene, the LAG3 gene, the CISH gene, the TGFBR2 gene, the FAS gene, and/or the CD7 gene.

For example, in some embodiments, the present disclosure provides a method for reducing immunogenicity and/or reducing graft versus host (GvH) response comprising introducing into the cell a first gene editing system targeting the TRAC gene, and a second gene editing system targeting the PDCD1 gene, the B2M gene, the CD52 gene, the CTLA4 gene, the TIGIT gene, the TIM3 gene, the LAG3 gene, the CISH gene, the TGFBR2 gene, the FAS gene, and/or the CD7 gene.

For example, in some embodiments, the present disclosure provides a method for reducing immunogenicity and/or reducing graft versus host (GvH) response comprising introducing into the cell a first gene editing system targeting the CD7 gene, and a second gene editing system targeting the TRAC gene, the B2M gene, the CD52 gene, the CTLA4 gene, the as TIGIT gene, the TIM3 gene, the LAG3 gene, the CISH gene, the TGFBR2 gene, the FAS gene, and/or the PDCD1 gene.

For example, in some embodiments, the present disclosure provides a method for reducing immunogenicity and/or reducing graft versus host (GvH) response comprising introducing into the cell a first gene editing system targeting the TRAC gene, and a second gene editing system targeting a gene selected from CD52, B2M, PDCD1, CTLA4, TIGIT, TIM3, LAG3, CISH, TGFBR2, FAS, CD7, CBLB, and CD38.

For example, in some embodiments, the present disclosure provides a method for reducing immunogenicity and/or reducing graft versus host (GvH) response comprising introducing into the cell a first gene editing system targeting the CD52 gene, and a second gene editing system targeting a gene selected from PDCD1, CTLA4, TIGIT, TIM3, LAG3, CISH, TGFBR2, FAS, CD7, CBLB, KLRC1, and CD38.

For example, in some embodiments, the present disclosure provides a method for reducing immunogenicity and/or reducing graft versus host (GvH) response comprising introducing into the cell a first gene editing system targeting the B2M gene, and a second gene editing system targeting a gene selected from PDCD1, CTLA4, TIGIT, TIM3, LAG3, CISH, TGFBR2, FAS, CD7, CBLB, KLRC1, and CD38.

For example, in some embodiments, the present disclosure provides a method for reducing immunogenicity and/or reducing graft versus host (GvH) response comprising introducing into the cell a first gene editing system targeting the PDCD1 gene, and a second gene editing system targeting a gene selected from CTLA4, TIGIT, TIM3, LAG3, CISH, TGFBR2, FAS, CD7, CBLB, KLRC1, and CD38.

For example, in some embodiments, the present disclosure provides a method for reducing immunogenicity and/or reducing graft versus host (GvH) response comprising introducing into the cell a first gene editing system targeting the CTLA4 gene, and a second gene editing system targeting a gene selected from TIGIT, TIM3, LAG3, CISH, TGFBR2, FAS, CD7, CBLB, KLRC1, and CD38.

For example, in some embodiments, the present disclosure provides a method for reducing immunogenicity and/or reducing graft versus host (GvH) response comprising introducing into the cell a first gene editing system targeting the TIGIT gene, and a second gene editing system targeting a gene selected from TIM3, LAG3, CISH, TGFBR2, FAS, CD7, CBLB, KLRC1, and CD38.

For example, in some embodiments, the present disclosure provides a method for reducing immunogenicity and/or reducing graft versus host (GvH) response comprising introducing into the cell a first gene editing system targeting the TIM3 gene, and a second gene editing system targeting a gene selected from LAG3, CISH, TGFBR2, FAS, CD7, CBLB, KLRC1, and CD38.

For example, in some embodiments, the present disclosure provides a method for reducing immunogenicity and/or reducing graft versus host (GvH) response comprising as introducing into the cell a first gene editing system targeting the LAG3 gene, and a second gene editing system targeting a gene selected from CISH, TGFBR2, FAS, CD7, CBLB, KLRC1, and CD38.

For example, in some embodiments, the present disclosure provides a method for reducing immunogenicity and/or reducing graft versus host (GvH) response comprising introducing into the cell a first gene editing system targeting the CISH gene, and a second gene editing system targeting a gene selected from TGFBR2, FAS, CD7, CBLB, KLRC1, and CD38.

For example, in some embodiments, the present disclosure provides a method for reducing immunogenicity and/or reducing graft versus host (GvH) response comprising introducing into the cell a first gene editing system targeting the TGFBR2 gene, and a second gene editing system targeting a gene selected from FAS, CD7, CBLB, KLRC1, and CD38.

For example, in some embodiments, the present disclosure provides a method for reducing immunogenicity and/or reducing graft versus host (GvH) response comprising introducing into the cell a first gene editing system targeting the FAS gene, and a second gene editing system targeting a gene selected from CD7, CBLB, KLRC1, and CD38.

For example, in some embodiments, the present disclosure provides a method for reducing immunogenicity and/or reducing graft versus host (GvH) response comprising introducing into the cell a first gene editing system targeting the CD7 gene, and a second gene editing system targeting a gene selected from CBLB and KLRC1.

For example, in some embodiments, the present disclosure provides a method for reducing immunogenicity and/or reducing graft versus host (GvH) response comprising introducing into the cell a first gene editing system targeting the CBLB gene, and a second gene editing system targeting a gene selected from KLRC1 and CD38.

For example, in some embodiments, the present disclosure provides a method for reducing immunogenicity and/or reducing graft versus host (GvH) response comprising introducing into the cell a first gene editing system targeting the KLRC1 gene, and a second gene editing system targeting CD38.

For example, in some embodiments, the present disclosure provides a method for reducing immunogenicity and/or reducing graft versus host (GvH) response comprising introducing into the cell a first gene editing system targeting the KLRC1 gene, and a second gene editing system targeting CD38.

For example, in some embodiments, the present disclosure provides a method for reducing immunogenicity and/or reducing graft versus host (GvH) response comprising introducing into the cell a first gene editing system targeting the KLRC1 gene, and a second gene editing system targeting a gene selected from PD1, TGFBR2, CISH, CD38, CBLB, TIGIT, TIM-3, LAG3, FAS, and TGFBR2.

For example, in some embodiments, the present disclosure provides a method for reducing immunogenicity and/or reducing graft versus host (GvH) response comprising knocking out two, three, or four of TRAC, CD52, B2M, PDCD1, CTLA4, TIGIT, TIM3, LAG3, CISH, as TGFBR2, FAS, CD7, CBLB, KLRC1, and/or CD38 with the corresponding gene editing systems disclosed herein. For example, in some embodiments, the present disclosure provides a method for reducing immunogenicity and/or reducing graft versus host (GvH) response comprising introducing into the cell a first gene editing system targeting the TRAC gene, a second gene editing system targeting the CD52 gene, a third gene editing system targeting the PDCD1 gene, and a fourth gene editing system targeting the CD7 gene.

TABLE 12
mgRNAs spacer and hgRNAs spacer

| mg1-TRAC-Exon1-AG1 | AUCUGUGGGACAAGAGGAUC | SEQ ID NO: 1 |
|---|---|---|
| mg2-TRAC-Exon1-AG2 | CUGGAUAUCUGUGGGACAAG | SEQ ID NO: 2 |
| mg3-TRAC-Exon1-GT | CUUACCUGGGCUGGGGAAGA | SEQ ID NO: 3 |
| mg4-TRAC-Exon3-AG | UUCGUAUCUGUAAAACCAAG | SEQ ID NO: 4 |
| mg5-TRAC-Exon3-CAG | UUUCAAAACCUGUCAGUGAU | SEQ ID NO: 5 |
| mg1-CD52-Exon1-GT1 | UUACCUGUACCAUAACCAGG | SEQ ID NO: 6 |
| mg2-CD52-Exon1-GT2 | CUCUUACCUGUACCAUAACC | SEQ ID NO: 7 |
| mg3-CD52-Exon2-AG | GUAUCUGUAGGAGGAGAAGU | SEQ ID NO: 8 |
| mg1-B2M-Exon1-GT | ACUCACGCUGGAUAGCCUCC | SEQ ID NO: 9 |
| mg2-B2M-Exon3-AG | UCGAUCUAUGAAAAAGACAG | SEQ ID NO: 10 |
| mg3-B2M-Exon2-CAG-NGN | CAAAGAUUCAGGUUUACUCA | SEQ ID NO: 11 |
| mg4-B2M-Exon2-CAG-NGN | ACUUUGUCACAGCCCAAGAU | SEQ ID NO: 12 |
| mg5-B2M-Exon2-CAG-NGN | GUCACAGCCCAAGAUAGUUA | SEQ ID NO: 13 |
| mg6-B2M-Exon2-CAG-NGN | CACAGCCCAAGAUAGUUAAG | SEQ ID NO: 14 |
| mg7-B2M-Exon2-TGG-NGN | AAAGACCAGUCCUUGCUGAA | SEQ ID NO: 15 |
| mg8-B2M-Exon2-TGG-NGN | AUAGAAAGACCAGUCCUUGC | SEQ ID NO: 16 |
| mg9-B2M-Exon2-TGG-NGN | UUACCCCACUUAACUAUCUU | SEQ ID NO: 17 |
| mg10-B2M-Exon2-TGG-NGN | CUUACCCCACUUAACUAUCU | SEQ ID NO: 18 |
| mg11-B2M-Exon2-AG | UGGAGUACCUGAGGAAUAUC | SEQ ID NO: 19 |
| mg1-PDCD1-Exon1-CAG | UGCAGAUCCCACAGGCGCCC | SEQ ID NO: 20 |
| mg2-PDCD1-Exon1-TGG1 | CGACUGGCCAGGGCGCCUGU | SEQ ID NO: 21 |
| mg3-PDCD1-Exon1-TGG2 | ACCGCCCAGACGACUGGCCA | SEQ ID NO: 22 |
| mg4-PDCD1-Exon1-CAA | CGGUGCUACAACUGGGCUGG | SEQ ID NO: 23 |
| mg5-PDCD1-Exon1-GT | CACCUACCUAAGAACCAUCC | SEQ ID NO: 24 |
| mg6-PDCD1-Exon2-AG | GGAGUCUGAGAGAUGGAGAG | SEQ ID NO: 25 |
| mg7-PDCD1-Exon2-TGG | GGGGUUCCAGGGCCUGUCUG | SEQ ID NO: 26 |
| mg8-PDCD1-Exon2-CAG1 | CAGCAACCAGACGGACAAGC | SEQ ID NO: 27 |
| mg9-PDCD1-Exon2-CAG2 | GGACCGCAGCCAGCCCGGCC | SEQ ID NO: 28 |
| mg10-PDCD1-Exon2-CAA | GUGUCACACAACUGCCCAAC | SEQ ID NO: 29 |
| mg11-PDCD1-Exon2-CAG3 | CGCAGAUCAAAGAGAGCCUG | SEQ ID NO: 30 |
| mg12-PDCD1-Exon3-AG | UUCUCUCUGGAAGGGCACAA | SEQ ID NO: 31 |
| mg13-PDCD1-Exon3-CAG | AGCCGGCCAGUUCCAAACCC | SEQ ID NO: 32 |
| mg14-PDCD1-Exon3-CAA | CAGUUCCAAACCCUGGUGG | SEQ ID NO: 33 |
| mg15-PDCD1-Exon3-TGG | GGACCCAGACUAGCAGCACC | SEQ ID NO: 34 |
| mg16-PDCD1-Exon4-AG | UCCCUGCAGAGAAACACACU | SEQ ID NO: 35 |
| mg17-PDCD1-Exon4-GT | GAGACUCACCAGGGGCUGGC | SEQ ID NO: 36 |
| mg18-PDCD1-Exon5-AG1 | UUCUUUGAGGAGAAAGGGAG | SEQ ID NO: 37 |
| mg19-PDCD1-Exon5-AG2 | CCUCCUUCUUUGAGGAGAAA | SEQ ID NO: 38 |
| hg-mg1&2-U1-TRAC | AGAGUCUCUCAGCUGGUACA | SEQ ID NO: 39 |
| hg-mg1&2-U2-TRAC | UGGAUUUAGAGUCUCUCAGC | SEQ ID NO: 40 |
| hg-mg1&2-U3-TRAC | UAGGCAGACAGACUUGUCAC | SEQ ID NO: 41 |
| hg-mg3-U1-TRAC | ACCUGGCCAUUCCUGAAGCA | SEQ ID NO: 42 |
| hg-mg3-U2-TRAC | CCAGAGCUCUGGGCAGAACC | SEQ ID NO: 43 |
| hg-mg3-U3-TRAC | ACAUCAUUGACCAGAGCUCU | SEQ ID NO: 44 |
| hg-mg4-U1-TRAC | UUCGGAACCCAAUCACUGAC | SEQ ID NO: 45 |
| hg-mg4-U2-TRAC | CCACUUUCAGGAGGAGGAUU | SEQ ID NO: 46 |
| hg-mg4-U3-TRAC | ACCCGGCCACUUUCAGGAGG | SEQ ID NO: 47 |
| hg-mg5-U1-TRAC | CCAUAACCGCUGUGGCCUCU | SEQ ID NO: 48 |
| hg-mg5-U2-TRAC | AUGCAAGCCCAUAACCGCUG | SEQ ID NO: 49 |
| hg-mg5-U3-TRAC | CCUGCAGGCCAUGCAGGCCU | SEQ ID NO: 50 |
| hg-mg1&2-U1-CD52 | CAAGCAACUUUGGGGAGUCC | SEQ ID NO: 51 |
| hg-mg1&2-U2-CD52 | CUCCAUGCCAAGCAACUUUG | SEQ ID NO: 52 |
| hg-mg1&2-U3-CD52 | CCCUCCAUGCCAAGCAACUU | SEQ ID NO: 53 |
| hg-mg3-U1-CD52 | UGAGGGGCUGCUGGUUUGGC | SEQ ID NO: 54 |
| hg-mg3-U2-CD52 | GCUGGAUGCUGAGGGGCUGC | SEQ ID NO: 55 |
| hg-mg3-U3-CD52 | UUAUGUUGCUGGAUGCUGAG | SEQ ID NO: 56 |
| hg-mg1-U1-B2M | GAGGAAGGACCAGAGCGGGA | SEQ ID NO: 57 |
| hg-mg1-U2-B2M | GGGAGAGGAAGGACCAGAGC | SEQ ID NO: 58 |
| hg-mg1-U3-B2M | GGGUGCAGAGCGGGAGAGGA | SEQ ID NO: 59 |
| hg-mg1-U4-B2M | GGGCCACAGAGGGUGCAGAG | SEQ ID NO: 60 |
| hg-mg1-U5-B2M | CAGAGGGUGCAGAGCGGGAG | SEQ ID NO: 61 |
| hg-mg2-U1-B2M | GAAACAAAAACAUUUUCUCA | SEQ ID NO: 62 |
| hg-mg3-U1-B2M | UCUUUUCCCGAUAUUCCUCA | SEQ ID NO: 63 |
| hg-mg3-U2-B2M | AAUAUUAAUGUGUCUUUUCC | SEQ ID NO: 64 |
| hg-mg3-U3-B2M | AUACCCUGGCAAUAUUAAUG | SEQ ID NO: 65 |
| hg-mg3-U4-B2M | AGCCCCAAGUGAAAUACCCU | SEQ ID NO: 66 |
| hg-mg4&5&6-U1-B2M | UAUGCCUGCCGUGUGAACCA | SEQ ID NO: 67 |
| hg-mg4&5&6-U2-B2M | AGAUGAGUAUGCCUGCCGUG | SEQ ID NO: 68 |
| hg-mg4&5&6-U3-B2M | CUGAAAAAGAUGAGUAUGCC | SEQ ID NO: 69 |
| hg-mg4&5&6-U4-B2M | CACCCCCACUGAAAAAGAUG | SEQ ID NO: 70 |
| hg-mg4&5&6-U5-B2M | UACACUGAAUUCACCCCCAC | SEQ ID NO: 71 |
| hg-mg7&8-U1-B2M | GGUGAAUUCAGUGUAGUACA | SEQ ID NO: 72 |
| hg-mg7&8-U2-B2M | AGUGGGGGUGAAUUCAGUGU | SEQ ID NO: 73 |
| hg-mg7&8-U3-B2M | UUUUCAGUGGGGGUGAAUUC | SEQ ID NO: 74 |
| hg-mg7&8-U4-B2M | CUCAUCUUUUUCAGUGGGGG | SEQ ID NO: 75 |
| hg-mg7&8-U5-B2M | CAGGCAUACUCAUCUUUUUC | SEQ ID NO: 76 |
| hg-mg9&10-U1-B2M | UUCAGCAGCUUACAAAAGAA | SEQ ID NO: 77 |
| hg-mg9&10-U2-B2M | AACUUUCAGCAGCUUACAAA | SEQ ID NO: 78 |
| hg-mg9&10-U3-B2M | ACUCAUACACAACUUUCAGC | SEQ ID NO: 79 |
| hg-mg9&10-U4-B2M | ACUACUCAUACACAACUUUC | SEQ ID NO: 80 |
| hg-mg11-U1-B2M | UUUGACUUUCCAUUCUCUGC | SEQ ID NO: 81 |
| hg-mg11-U2-B2M | ACCCAGACACAUAGCAAUUC | SEQ ID NO: 82 |
| hg-mg1-U1-PDCD1 | CAGUGGAGAAGGCGGCACUC | SEQ ID NO: 83 |
| hg-mg1-U2-PDCD1 | CGCCUGAGCAGUGGAGAAGG | SEQ ID NO: 84 |
| hg-mg1-U3-PDCD1 | CUCCGCCUGAGCAGUGGAGA | SEQ ID NO: 85 |
| hg-mg1-U4-PDCD1 | GCUCACCUCCGCCUGAGCAG | SEQ ID NO: 86 |
| hg-mg2&3-U1-PDCD1 | CACCUACCUAAGAACCAUCC | SEQ ID NO: 87 |
| hg-mg4-U1-PDCD1 | UGCAGAUCCCACAGGCGCCC | SEQ ID NO: 88 |
| hg-mg4-U2-PDCD1 | UCCAGGCAUGCAGAUCCCAC | SEQ ID NO: 89 |
| hg-mg4-U3-PDCD1 | CACUCUGGUGGGGCUGCUCC | SEQ ID NO: 90 |
| hg-mg5-U1-PDCD1 | CCCUCCAGACCCCUGGCUCU | SEQ ID NO: 91 |
| hg-mg5-U2-PDCD1 | GGAAGGUCCCUCCAGACCCC | SEQ ID NO: 92 |
| hg-mg5-U3-PDCD1 | UGCCAGGGACUGAGGGUGGA | SEQ ID NO: 93 |
| hg-mg6-U1-PDCD1 | AGCAGGGCUGGGGAGAAGGU | SEQ ID NO: 94 |
| hg-mg6-U2-PDCD1 | CACGAGCAGGGCUGGGGAGA | SEQ ID NO: 95 |
| hg-mg6&7-U3-PDCD1 | CCCUUCGGUCACCACGAGCA | SEQ ID NO: 96 |
| hg-mg7-U1-PDCD1 | GGAGAAGCUGCAGGUGAAGG | SEQ ID NO: 97 |
| hg-mg7-U2-PDCD1 | GAAGGUGGCGUUGUCCCCUU | SEQ ID NO: 98 |
| hg-mg8&9-U1-PDCD1 | CGGAGAGCUUCGUGCUAAAC | SEQ ID NO: 99 |
| hg-mg8-U2-PDCD1 | CUGCAGCUUCUCCAACACAU | SEQ ID NO: 100 |
| hg-mg9-U2-PDCD1 | CAUGAGCCCCAGCAACCAGA | SEQ ID NO: 101 |
| hg-mg9-U3-PDCD1 | CAGCAACCAGACGGACAAGC | SEQ ID NO: 102 |
| hg-mg10-U1-PDCD1 | GGACCGCAGCCAGCCCGGCC | SEQ ID NO: 103 |
| hg-mg10-U2-PDCD1 | CCCGAGGACCGCAGCCAGCC | SEQ ID NO: 104 |
| hg-mg10-U3-PDCD1 | CAAGCUGGCCGCCUUCCCCG | SEQ ID NO: 105 |
| hg-mg11-U1-PDCD1 | ACAGCGGCACCUACCUCUGU | SEQ ID NO: 106 |
| hg-mg11-U2-PDCD1 | AGGGCCCGGCGCAAUGACAG | SEQ ID NO: 107 |
| hg-mg11-U3-PDCD1 | ACAUGAGCGUGGUCAGGGCC | SEQ ID NO: 108 |
| hg-mg12-U1-PDCD1 | CCUGGGUGAGGGGCUGGGGU | SEQ ID NO: 109 |
| hg-mg12-U2-PDCD1 | GGCUGGCCUGGGUGAGGGGC | SEQ ID NO: 110 |
| hg-mg12-U3-PDCD1 | UGGCCGGCUGGCCUGGGUGA | SEQ ID NO: 111 |
| hg-mg13&14-U1-PDCD1 | UUGUGCCCUUCCAGAGAGAA | SEQ ID NO: 112 |
| hg-mg15-U1-PDCD1 | UGACGUUACCUCGUGCGGCC | SEQ ID NO: 113 |
| hg-mg15-U2-PDCD1 | UGGGAUGACGUUACCUCGUG | SEQ ID NO: 114 |
| hg-mg15-U3-PDCD1 | GCAGGGCAGGCCGAGGGGCU | SEQ ID NO: 115 |
| hg-mg16-U1-PDCD1 | GAGACUCACCAGGGGCUGGC | SEQ ID NO: 116 |
| hg-mg16-U2-PDCD1 | GAGUGAGACUCACCAGGGGC | SEQ ID NO: 117 |
| hg-mg16-U3-PDCD1 | GAAAAGAGUGAGACUCACCA | SEQ ID NO: 118 |
| hg-mg17-U1-PDCD1 | ACCCAGGAAGGAAGGCACAG | SEQ ID NO: 119 |
| hg-mg17-U2-PDCD1 | CUCUGCCCACCCAGGAAGGA | SEQ ID NO: 120 |
| hg-mg17-U3-PDCD1 | CCACCUCUGCCCACCCAGGA | SEQ ID NO: 121 |
| hg-mg18&19-U1-PDCD1 | CCAUAGUCCACAGAGAACAC | SEQ ID NO: 122 |
| hg-mg18&19-U2-PDCD1 | CCGGGGUCUUCUCUCGCCAC | SEQ ID NO: 123 |
| mg1-CTLA4-Exon1-CAG | CUCAGCUGAACCUGGCUACC | SEQ ID NO: 247 |
| mg2-CTLA4-Exon1-TGG | AGGGCCAGGUCCUGGUAGCC | SEQ ID NO: 248 |
| mg3-CTLA4-Exon1-GT | CACUCACCUUUGCAGAAGAC | SEQ ID NO: 249 |
| mg4-CTLA4-Exon2-AG | UGCUAGCAUGGAAAAGCCAA | SEQ ID NO: 250 |
| mg5-CTLA4-Exon2-CAG1 | GGCCCAGCCUGCUGUGGUAC | SEQ ID NO: 251 |
| mg6-CTLA4-Exon2-CAG2 | GCUUCGGCAGGCUGACAGCC | SEQ ID NO: 252 |
| mg7-CTLA4-Exon2-CAG3 | CCAGGUGACUGAAGUCUGUG | SEQ ID NO: 253 |
| mg8-CTLA4-Exon2-CAA | UAUCCAAGGACUGAGGGCCA | SEQ ID NO: 254 |
| mg9-CTLA4-Exon2-CAG4 | GGAACCCAGAUUUAUGUAAU | SEQ ID NO: 255 |
| mg10-CTLA4-Exon2-GT | CUCACCAAUUACAUAAAUCU | SEQ ID NO: 256 |
| hg-mg1-U1-CTLA4 | CAUAAAGCCAUGGCUUGCCU | SEQ ID NO: 257 |
| hg-mg1-U2-CTLA4 | ACACCGCUCCCAUAAAGCCA | SEQ ID NO: 258 |
| hg-mg2-U1-CTLA4 | ACUCACCUUUGCAGAAGACA | SEQ ID NO: 259 |
| hg-mg3-U1-CTLA4 | GAAACAAAUGAAACCCAGGU | SEQ ID NO: 260 |
| hg-mg3-U2-CTLA4 | UGCUGAAACAAAUGAAACCC | SEQ ID NO: 261 |
| hg-mg4-U1-CTLA4 | CUGCUGGCCAGUACCACAGC | SEQ ID NO: 262 |
| hg-mg4-U2-CTLA4 | GCUGGCGAUGCCUCGGCUG | SEQ ID NO: 263 |
| hg-mg4-U3-CTLA4 | ACACAAAGCUGGCGAUGCCU | SEQ ID NO: 264 |
| hg-mg5-U1-CTLA4 | UGAGUUCACUGAGUUCCCUU | SEQ ID NO: 265 |
| hg-mg5-U2-CTLA4 | UUGCCUGGGCUUGGCCAUGA | SEQ ID NO: 266 |
| hg-mg5-U3-CTLA4 | CAGAAGGGCUUGCCUGGGCU | SEQ ID NO: 267 |
| hg-mg6&7-U1-CTLA4 | AUCUCCAGGCAAAGCCACUG | SEQ ID NO: 268 |
| hg-mg6&7-U2-CTLA4 | GUGUGUGAGUAUGCAUCUCC | SEQ ID NO: 269 |
| hg-mg7-U3-CTLA4 | AGGCAAAGCCACUGAGGUCC | SEQ ID NO: 270 |
| hg-mg8-U1-CTLA4 | AUCUGCACGGGCACCUCCAG | SEQ ID NO: 271 |
| hg-mg8-U2-CTLA4 | CUAGAUGAUUCCAUCUGCAC | SEQ ID NO: 272 |
| hg-mg9-U1-CTLA4 | GUACCCACCGCCAUACUACC | SEQ ID NO: 273 |
| hg-mg9-U2-CTLA4 | GGGACUCUACAUCUGCAAGG | SEQ ID NO: 274 |
| hg-mg9-U3-CTLA4 | CACGGGACUCUACAUCUGCA | SEQ ID NO: 275 |
| hg-mg10-U1-CTLA4 | AUAGAAGACUGCAAUGCAAC | SEQ ID NO: 276 |
| hg-mg10-U2-CTLA4 | AAGUAAACCUCCUGAAAUUA | SEQ ID NO: 277 |
| mg1-TIGIT-Exon1-TGG-NG | GAGACACCAGCGCAUGCUUC | SEQ ID NO: 278 |
| mg2-TIGIT-Exon1-TGG-NG | CCUGGGCCCAGAUCAGGAGG | SEQ ID NO: 279 |
| mg3-TIGIT-Exon1-CAG | CUGGGCCCAGGGGCUGAGGC | SEQ ID NO: 280 |
| mg4-TIGIT-Exon1-CAG | AGGCAGGCUCCCCUCGCCUC | SEQ ID NO: 281 |
| mg5-TIGIT-Exon1-GT-NG | GCCUUACCUGAGGCGAGGGG | SEQ ID NO: 282 |
| mg6-TIGIT-Exon1-GT | CAGGCCUUACCUGAGGCGAG | SEQ ID NO: 283 |
| mg7-TIGIT-Exon2-AG-NG | AUUCCUAGGGAAGAGGACGA | SEQ ID NO: 284 |
| mg8-TIGIT-Exon2-CAA | CACAAGUGACCCAGGUCAAC | SEQ ID NO: 285 |
| mg9-TIGIT-Exon2-CAG-NG | AGUGACCCAGGUCAACUGGG | SEQ ID NO: 286 |
| mg10-TIGIT-Exon2-CAG-NG | GACCCAGGUCAACUGGGAGC | SEQ ID NO: 287 |
| mg11-TIGIT-Exon2-CAG | GGAGCAGCAGGACCAGCUUC | SEQ ID NO: 288 |
| mg12-TIGIT-Exon2-CAG-NG | AGGACCAGCUUCUGGCCAUU | SEQ ID NO: 289 |
| mg13-TIGIT-Exon2-TGG-NG | GAGAUGUGCCACCCCAAGUC | SEQ ID NO: 290 |
| mg14-TIGIT-Exon2-CGA | GAUCGAGUGGCCCCAGGUCC | SEQ ID NO: 291 |
| mg15-TIGIT-Exon2-CAG-NG | CACCCUCCAGUCGCUGACCG | SEQ ID NO: 292 |
| mg16-TIGIT-Exon2-CAG-NG | CUCCAGUCGCUGACCGUGAA | SEQ ID NO: 293 |
| mg17-TIGIT-Exon2-GT | CAGGAAUACCUGAGCUUUCU | SEQ ID NO: 294 |
| hg-mg1&2-U1-TIGIT | CAGGCCUUACCUGAGGCGAG | SEQ ID NO: 295 |
| hg-mg1&2-U2-TIGIT | UGGGUUUCAGGCCUUACCUG | SEQ ID NO: 296 |
| hg-mg1&2-U3-TIGIT | CUGCUGCUCUGCUGGGUUUC | SEQ ID NO: 297 |
| hg-mg3&4-U1-TIGIT | CUCUGGGCAGAAGCAUGCGC | SEQ ID NO: 298 |
| hg-mg3&4-U2-TIGIT | CUGCUUCCUGUAGGCCCUCU | SEQ ID NO: 299 |
| hg-mg3&4-U3-TIGIT | AGGCCACAUCUGCUUCCUGU | SEQ ID NO: 300 |
| hg-mg5&6-U1-NG-TIGIT | GCCUUGUUUUUCCUCCCUGC | SEQ ID NO: 301 |
| hg-mg5&6-U2-NG-TIGIT | UCUCCAACCAAGCUUAGCCU | SEQ ID NO: 302 |
| hg-mg5&6-U3-NG-TIGIT | AGCACCCAAGUCUCCAACCA | SEQ ID NO: 303 |
| hg-mg7-U1-NG-TIGIT | UUCUCUGCAGAAAUGUUCCC | SEQ ID NO: 304 |
| hg-mg7-U2-NG-TIGIT | AUAGAGCCACCUUUCUCUGC | SEQ ID NO: 305 |
| hg-mg9&10-U1-NG-TIGIT | GUGGCUCUAUCAUCUUACAA | SEQ ID NO: 306 |
| hg-mg8&9&10-U2-TIGIT | AACAUUUCUGCAGAGAAAGG | SEQ ID NO: 307 |
| hg-mg8&9&10-U3-TIGIT | GGGAACAUUUCUGCAGAGAA | SEQ ID NO: 308 |
| hg-mg11&12-U1-NG-TIGIT | GUGGCUCUAUCAUCUUACAA | SEQ ID NO: 309 |
| hg-mg11&12-U2-TIGIT | AUGUCACCUCUCCUCCACCA | SEQ ID NO: 310 |
| hg-mg12-U3-TIGIT | CACCACGGCACAAGUGACCC | SEQ ID NO: 311 |
| hg-mg13-U1-TIGIT | GAGGCCCAGGCCGGGACCUG | SEQ ID NO: 312 |
| hg-mg13-U2-TIGIT | UGGAGGGUGAGGCCCAGGCC | SEQ ID NO: 313 |
| hg-mg13-U3-TIGIT | GCGACUGGAGGGUGAGGCCC | SEQ ID NO: 314 |
| hg-mg14-U1-TIGIT | UUUGUAAUGCUGACUUGGGG | SEQ ID NO: 315 |
| hg-mg14-U2-TIGIT | CCAUUUGUAAUGCUGACUUG | SEQ ID NO: 316 |
| hg-mg14-U3-TIGIT | GGCCAUUUGUAAUGCUGACU | SEQ ID NO: 317 |
| hg-mg15&16-U1-TIGIT | UUCAAGGAUCGAGUGGCCCC | SEQ ID NO: 318 |
| hg-mg15&16-U2-TIGIT | CCCAUCCUUCAAGGAUCGAG | SEQ ID NO: 319 |
| hg-mg15&16-U3-TIGIT | GCACAUCUCCCCAUCCUUCA | SEQ ID NO: 320 |
| hg-mg17-U1-TIGIT | AUUUUCUAUGCUAGAGGGAG | SEQ ID NO: 321 |
| hg-mg17-U2-TIGIT | AUUGCAUUUUCUAUGCUAGA | SEQ ID NO: 322 |
| mg1-TIM-3-Exon2-AG | GGACCCUGCAUAGAGAGAGA | SEQ ID NO: 323 |
| mg2-TIM-3-Exon2-CAG-NG | GGUCGGUCAGAAUGCCUAUC | SEQ ID NO: 324 |
| mg3-TIM-3-Exon2-CAG-NG | GUCAGAAUGCCUAUCUGCCC | SEQ ID NO: 325 |
| mg4-TIM-3-Exon2-TGG | UGCCCCAGCAGACGGGCACG | SEQ ID NO: 326 |
| mg5-TIM-3-Exon2-TGG-NG | CCAUUUAGCCAGUAUCUGGA | SEQ ID NO: 327 |
| mg6-TIM-3-Exon2-CAA-NG | GAUCCAAAUCCCAGGCAUAA | SEQ ID NO: 328 |
| mg7-TIM-3-Exon3-CAG-NG | ACUCGGCAGAGAGACUUCAC | SEQ ID NO: 329 |
| mg8-TIM-3-Exon3-CAG-NG | CGGCAGAGAGACUUCACUGC | SEQ ID NO: 330 |
| mg9-TIM-3-Exon3-GT-NG | GUUACCUGGGCCAUGUCCCC | SEQ ID NO: 331 |
| mg11-TIM-3-Exon4-AG-NG | UGCUAUAAAAAGAGAGAGAG | SEQ ID NO: 332 |
| mg12-TIM-3-Exon4-AG-NG | UCUGCUAUAAAAAGAGAGAG | SEQ ID NO: 333 |
| mg13-TIM-3-Exon4-AG-NG | UCUCUGCUAUAAAAAGAGAG | SEQ ID NO: 334 |
| mg14-TIM-3-Exon4-CAG-NG | CAGAGACACAGACACUGGGG | SEQ ID NO: 335 |
| mg15-TIM-3-Exon4-GT-NG | UUACUGUUAGAUUUAUAUCA | SEQ ID NO: 336 |
| mg16-TIM-3-Exon4-GT-NG | CUUACUGUUAGAUUUAUAUC | SEQ ID NO: 337 |
| hg-mg1-U1-TIM-3 | GGUGUAGAAGCAGGGCAGAU | SEQ ID NO: 338 |
| hg-mg1-U2-TIM-3 | GCGGCUGGGGUGUAGAAGCA | SEQ ID NO: 339 |
| hg-mg2&3-U1-TIM-3 | CUCACUCACCGCUUGAGUCU | SEQ ID NO: 340 |
| hg-mg2&3-U2-TIM-3 | UCUCCUUCUCUCUCUAUGCA | SEQ ID NO: 341 |
| hg-mg3-U3-TIM-3 | CUAUGCAGGGUCCUCAGAAG | SEQ ID NO: 342 |
| hg-mg4-U1-TIM-3 | ACGUUGCCACAUUCAAACAC | SEQ ID NO: 343 |
| hg-mg4-U2-TIM-3 | AUCAGUCCUGAGCACCACGU | SEQ ID NO: 344 |
| hg-mg4-U3-TIM-3 | CACAUCCCUUUCAUCAGUCC | SEQ ID NO: 345 |
| hg-mg5-U1-TIM-3 | AGUCACAUUCUCUAUGGUCA | SEQ ID NO: 346 |
| hg-mg5-U2-TIM-3 | UGCUAGAGUCACAUUCUCUA | SEQ ID NO: 347 |
| hg-mg6-U1-TIM-3 | GUCCCUGACCAUAGAGAAUG | SEQ ID NO: 348 |
| hg-mg6-U2-TIM-3 | ACCAUAGAGAAUGUGACUCU | SEQ ID NO: 349 |
| hg-mg6-U3-TIM-3 | AAUGUGACUCUAGCAGACAG | SEQ ID NO: 350 |
| hg-mg7&8-U1-TIM-3 | UCAGAUAAUUUUCAUCCUUA | SEQ ID NO: 351 |
| hg-mg7&8-U2-TIM-3 | UUUCAUCCUUAUGUUGUUUC | SEQ ID NO: 352 |
| hg-mg7&8-U3-TIM-3 | CUUAUGUUGUUUCUGACAUU | SEQ ID NO: 353 |
| hg-mg7&8-U4-TIM-3 | GUUGUUUCUGACAUUAGCCA | SEQ ID NO: 354 |
| hg-mg9-U1-TIM-3 | AUUCAAAAUCCUCUGAAUUC | SEQ ID NO: 355 |
| hg-mg9-U2-TIM-3 | UUCCCACAUUCAAAAUCCUC | SEQ ID NO: 356 |
| hg-mg11&12&13-U1-TIM-3 | CUGUUAGAUUUAUAUCAGGG | SEQ ID NO: 357 |
| hg-mg11&12&13-U2-TIM-3 | UUACUGUUAGAUUUAUAUCA | SEQ ID NO: 358 |
| hg-mg11&12&13-U3-TIM-3 | AAAAAAAGUUACUUACUGUU | SEQ ID NO: 359 |
| hg-mg14-U1-TIM-3 | AACGUUCUUAACUAUACUAU | SEQ ID NO: 360 |
| hg-mg15&16-U1-TIM-3 | AUGAUUUCCCCUCCAAGUUG | SEQ ID NO: 361 |
| hg-mg15&16-U1-TIM-3 | AAGCCAUGAUUUCCCCUCCA | SEQ ID NO: 362 |
| hg-mg15&16-U1-TIM-3 | AUGAAGGAAGUCUAAAGCCA | SEQ ID NO: 363 |
| mg1-LAG3-Exon1-TGG | AGCCUCCCACAUCUCUCCUA | SEQ ID NO: 364 |
| mg2-LAG3-Exon1-CAG | GUUUCUGCAGCCGCUUUGGG | SEQ ID NO: 365 |
| mg3-LAG3-Exon1-GT | UUACCUGGAGCCACCCAAAG | SEQ ID NO: 366 |
| mg4-LAG3-Exon2-AG | UCACUAGGUGAGCAAAAGAG | SEQ ID NO: 367 |
| mg5-LAG3-Exon2-CAG1 | GCCUCUCCAGCCAGGGGCUG | SEQ ID NO: 368 |
| mg7-LAG3-Exon2-CGA | UGCGAAGAGCAGGGGUCACU | SEQ ID NO: 369 |
| mg8-LAG3-Exon2-CAG2 | CUUGGCAGCAUCAGCCAGAC | SEQ ID NO: 370 |
| mg9-LAG3-Exon3-AG | CCACUGGGCGGGAAAGAGAA | SEQ ID NO: 371 |
| mg10-LAG3-Exon3-TGG | CCCCAGGAGGAGGGCGCCGC | SEQ ID NO: 372 |
| mg12-LAG3-Exon3-CAG1 | CCUGCAGCCCCGCGUCCAGC | SEQ ID NO: 373 |
| mg13-LAG3-Exon3-CAG2 | CGCGUCCAGCUGGAUGAGCG | SEQ ID NO: 374 |
| mg14-LAG3-Exon3-CAG3 | UGGGCCAGGCCUCGAGUAUG | SEQ ID NO: 375 |
| mg15-LAG3-Exon3-GT | ACAUACUCGAGGCCUGGCCC | SEQ ID NO: 376 |
| mg16-LAG3-Exon4-TGG1 | CGGAACCAAUGCACAGAGGC | SEQ ID NO: 377 |
| mg17-LAG3-Exon4-CGA | CCAGGGCCGAGUCCCUGUCC | SEQ ID NO: 378 |
| mg18-LAG3-Exon4-CAA | CCUGCCCCAAGUCAGCCCCA | SEQ ID NO: 379 |
| mg19-LAG3-Exon4-TGG2 | GCCCCAGGGCCCAGAGUCCA | SEQ ID NO: 380 |
| mg20-LAG3-Exon4-GT | GGGAGUUACCCAGAACAGUG | SEQ ID NO: 381 |
| mg11-LAG3-Exon5-TGG | GAGGAGUCCACUUGGCAGUG | SEQ ID NO: 382 |
| mg21-LAG3-Exon5-CAG | AUGUGAGCCAGGCCCAGGCU | SEQ ID NO: 383 |
| mg22-LAG3-Exon6-AG | GAGUCACUGAAAAGAGUAGA | SEQ ID NO: 384 |
| mg23-LAG3-Exon6-CAA | CUGGACAAGAACGCUUUGUG | SEQ ID NO: 385 |
| mg24-LAG3-Exon6-CAG | CCAUCCCAGAGGAGUUUCUC | SEQ ID NO: 386 |
| mg25-LAG3-Exon6-CAG | CCCAGCUCCUUUCCCAGCCU | SEQ ID NO: 387 |
| mg26-LAG3-Exon6-CAA | UUGGCAAUGCCAGCUGUACC | SEQ ID NO: 388 |
| mg27-LAG3-Exon6-CAG | GCCAGCUGUACCAGGGGGAG | SEQ ID NO: 389 |
| mg28-LAG3-Exon6-CAG | UACCAGGGGGAGAGGCUUCU | SEQ ID NO: 390 |
| mg29-LAG3-Exon7-AG | GGCACCUAUGGAGAAAGUAC | SEQ ID NO: 391 |
| mg30-LAG3-Exon7-CAG | AGACAGGUGAGCCAGGGACA | SEQ ID NO: 392 |
| mg31-LAG3-Exon7-GT | GGCUCACCUGUCUUCUCCAA | SEQ ID NO: 393 |
| mg32-LAG3-Exon8-AG&TGG | GUCGCCACUGUGAGAAGAGA | SEQ ID NO: 394 |
| mg33-LAG3-Exon8-CAG | GCAGGCUCAGAGCAAGAUAG | SEQ ID NO: 395 |
| mg34-LAG3-Exon8-CAA | GCUGGAGCAAGAACCGGAGC | SEQ ID NO: 396 |
| hg-mg1-U1-LAG3 | UUACCUGGAGCCACCCAAAG | SEQ ID NO: 397 |
| hg-mg1-U2-LAG3 | CCCGCCAUCCCCGUUUUACC | SEQ ID NO: 398 |
| hg-mg2-U1-LAG3 | GACCAUAGGAGAGAUGUGGG | SEQ ID NO: 399 |
| hg-mg2-U2-LAG3 | CCCAGACCAUAGGAGAGAUG | SEQ ID NO: 400 |
| hg-mg2-U3-LAG3 | CUCAGCGCUGCCCAGACCAU | SEQ ID NO: 401 |
| hg-mg3-U1-LAG3 | GUCCCCUCCUGUGGGGCUGG | SEQ ID NO: 402 |
| hg-mg3-U2-LAG3 | CCCUGCUGGUCCCCUCCUGU | SEQ ID NO: 403 |
| hg-mg3-U3-LAG3 | GUGGCCACAGAGAUCCCUGC | SEQ ID NO: 404 |
| hg-mg4-U1-LAG3 | CCUCCUGGGCCCACACCACC | SEQ ID NO: 405 |
| hg-mg4-U2-LAG3 | CUGGGCAGGAGCCCCCUCCU | SEQ ID NO: 406 |
| hg-mg4-U3-LAG3 | GGGCUGCAGGGGAGCUGGGC | SEQ ID NO: 407 |
| hg-mg5-U1-LAG3 | CGGCUCACGCCCCCUCCCCU | SEQ ID NO: 408 |
| hg-mg5-U2-LAG3 | CCUUCUCUACCCCUGCCUCU | SEQ ID NO: 409 |
| hg-mg7-U1-LAG3 | GGUGGUGUGGGCCCAGGAGG | SEQ ID NO: 410 |
| hg-mg8-U1-LAG3 | CAGCCCCACAAUCCCCCUCC | SEQ ID NO: 411 |
| hg-mg9-U1-LAG3 | GCCGGGGGCCAGGGGAUGGC | SEQ ID NO: 412 |
| hg-mg9-U2-LAG3 | CGGGUGAGGGCCGGGGGCCA | SEQ ID NO: 413 |
| hg-mg9-U3-LAG3 | GAGGAGGGCGCCGCCGGGUG | SEQ ID NO: 414 |
| hg-mg10-U1-LAG3 | CUCCCGCUGCGCAGGCCUCC | SEQ ID NO: 415 |
| hg-mg10-U2-LAG3 | GGGGCAGCCUCCCGCUGCGC | SEQ ID NO: 416 |
| hg-mg12&13-U1-LAG3 | GUGCUGAGCGUGGGUCCCGG | SEQ ID NO: 417 |
| hg-mg12&13-U2-LAG3 | CGCUACACGGUGCUGAGCGU | SEQ ID NO: 418 |
| hg-mg12-U3-LAG3 | GCCCAGGCCCCGCCGCUACA | SEQ ID NO: 419 |
| hg-mg13-U3-LAG3 | CCGGAGGCCUGCGCAGCGGG | SEQ ID NO: 420 |
| hg-mg14-U1-LAG3 | CCGCGCCGCGGUGCACCUCA | SEQ ID NO: 421 |
| hg-mg14-U2-LAG3 | CGCCGGCGAGUACCGCGCCG | SEQ ID NO: 422 |
| hg-mg15-U1-LAG3 | CGUCCCGGGAGGCAGGGGAU | SEQ ID NO: 423 |
| hg-mg15-U2-LAG3 | CUUCCUGCGUCCCGGGAGGC | SEQ ID NO: 424 |
| hg-mg15-U3-LAG3 | CCCAGCCCUUCCUGCGUCCC | SEQ ID NO: 425 |
| hg-mg16-U1-LAG3 | GGGACUCCCGGACAGGGACU | SEQ ID NO: 426 |
| hg-mg16-U2-LAG3 | AGUGGUGAUGGGGGGACUCC | SEQ ID NO: 427 |
| hg-mg16-U3-LAG3 | AGAGGAAGCUUUCCGCUAAG | SEQ ID NO: 428 |
| hg-mg17-U1-LAG3 | ACCGCCCAGCCUCUGUGCAU | SEQ ID NO: 429 |
| hg-mg18-U1-LAG3 | GUCCCCCCAUCACCACUUAG | SEQ ID NO: 430 |
| hg-mg18-U2-LAG3 | CCAGGGCCGAGUCCCUGUCC | SEQ ID NO: 431 |
| hg-mg19-U1-LAG3 | AACAGUGAGGUUAUACAUGA | SEQ ID NO: 432 |
| hg-mg19-U2-LAG3 | GGGAGUUACCCAGAACAGUG | SEQ ID NO: 433 |
| hg-mg20-U1-LAG3 | AGGGGGGCAGGAAGGAGUUG | SEQ ID NO: 434 |
| hg-mg20-U2-LAG3 | AGGUGACAAGGGGGGCAGGA | SEQ ID NO: 435 |
| hg-mg20-U3-LAG3 | AUAGUUAGGGGAGGUGACAA | SEQ ID NO: 436 |
| hg-mg11-U1-LAG3 | CAUUGUCUCCAGUCACCAGG | SEQ ID NO: 437 |
| hg-mg11-U2-LAG3 | CGCCAUUGUCUCCAGUCACC | SEQ ID NO: 438 |
| hg-mg11-U3-LAG3 | GCUCACAUCCUCUAGUCGAA | SEQ ID NO: 439 |
| hg-mg21-U1-LAG3 | CUCCUGGUGACUGGAGACAA | SEQ ID NO: 440 |
| hg-mg21-U2-LAG3 | GGCCCUGACCUCCUGGUGAC | SEQ ID NO: 441 |
| hg-mg21-U3-LAG3 | UGGGGGAGGCCCUGACCUCC | SEQ ID NO: 442 |
| hg-mg22-U1-LAG3 | AGCAGCUUCCCCAGGGAUCC | SEQ ID NO: 443 |
| hg-mg22-U2-LAG3 | CUCACAAAGCAGCUUCCCCA | SEQ ID NO: 444 |
| hg-mg22-U3-LAG3 | AAGCGUUCUUGUCCAGAUAC | SEQ ID NO: 445 |
| hg-mg23-U1-LAG3 | UUUGGGUCACCUGGAUCCCU | SEQ ID NO: 446 |
| hg-mg23-U2-LAG3 | CCCAAAUCCUUUGGGUCACC | SEQ ID NO: 447 |
| hg-mg24-U1-LAG3 | CUGGACAAGAACGCUUUGUG | SEQ ID NO: 448 |
| hg-mg24-U2-LAG3 | UGUGAGGUGACUCCAGUAUC | SEQ ID NO: 449 |
| hg-mg24-U3-LAG3 | CCUGGGGAAGCUGCUUUGUG | SEQ ID NO: 450 |
| hg-mg25&26-U1-LAG3 | CCAUCCCAGAGGAGUUUCUC | SEQ ID NO: 451 |
| hg-mg25-U2-LAG3 | CUCUGGACACCCCAUCCCAG | SEQ ID NO: 452 |
| hg-mg25-U3-LAG3 | ACGCUUUGUGUGGAGCUCUC | SEQ ID NO: 453 |
| hg-mg26&27&28-U2-LAG3 | ACCUUGGCUGGAGGCACAGG | SEQ ID NO: 454 |
| hg-mg26&27&28-U3-LAG3 | UUUCUCAGGACCUUGGCUGG | SEQ ID NO: 455 |
| hg-mg27&28-U1-LAG3 | AGAGGAGUUUCUCAGGACCU | SEQ ID NO: 456 |
| hg-mg29-U1-LAG3 | GGCCUGCUGGGAGGGCACCU | SEQ ID NO: 457 |
| hg-mg29-U2-LAG3 | CAGGAGGUGGCCUGCUGGGA | SEQ ID NO: 458 |
| hg-mg29-U3-LAG3 | GGAUGAGAAACAGCAGGAGG | SEQ ID NO: 459 |
| hg-mg30-U1-LAG3 | UCUCUGCUCCUUUUGGUGAC | SEQ ID NO: 460 |
| hg-mg30-U2-LAG3 | UGUCCUUUCUCUGCUCCUUU | SEQ ID NO: 461 |
| hg-mg30-U3-LAG3 | CUCCUGCUGUUUCUCAUCCU | SEQ ID NO: 462 |
| hg-mg31-U1-LAG3 | AGCGGGAGCUGCUGGGGGGC | SEQ ID NO: 463 |
| hg-mg31-U2-LAG3 | AUGGAAGAGCGGGAGCUGCU | SEQ ID NO: 464 |
| hg-mg31-U3-LAG3 | CACUCUGAGGAUGGAAGAGC | SEQ ID NO: 465 |
| hg-mg32-U1-LAG3 | AGGGUGAAUCCCUUGCUCUA | SEQ ID NO: 466 |
| hg-mg32-U2-LAG3 | UCUUGCUCUGAGCCUGCGGA | SEQ ID NO: 467 |
| hg-mg32-U3-LAG3 | UCUAUCUUGCUCUGAGCCUG | SEQ ID NO: 468 |
| hg-mg33&34-U1-LAG3 | GAUUUUCUGCCUUAGAGCAA | SEQ ID NO: 469 |
| hg-mg33&34-U2-LAG3 | CUCUCCAUCUCUUCUCACAG | SEQ ID NO: 470 |
| hg-mg34-U3-LAG3 | GCAAGGGAUUCACCCUCCGC | SEQ ID NO: 471 |
| mg1-CISH-Exon3-AG | GGACGAGGUCUAGAAGGCAG | SEQ ID NO: 472 |
| mg2-CISH-Exon3-CAG1 | GUCAUGCAGCCCUUGCCUGC | SEQ ID NO: 473 |
| mg3-CISH-Exon3-CAG1 | CAUGCAGCCCUUGCCUGCUG | SEQ ID NO: 474 |
| mg4-CISH-Exon3-CAG2 | CCAGACAGAGAGUGAGCCAA | SEQ ID NO: 475 |
| mg5-CISH-Exon3-GT1 | CAGACUCACCAGAUUCCCGA | SEQ ID NO: 476 |
| mg6-CISH-Exon3-GT2 | CUCACCAGAUUCCCGAAGGU | SEQ ID NO: 477 |
| mg7-CISH-Exon4-AG1&TGG1 | ACCAGCCUAGGCAAGUGCAG | SEQ ID NO: 478 |
| mg8-CISH-Exon4-AG2 | AGCCUAGGCAAGUGCAGAGG | SEQ ID NO: 479 |
| mg9-CISH-Exon4-TGG2 | UGGAACCCCAAUACCAGCCU | SEQ ID NO: 480 |
| mg10-CISH-Exon4-CAG1 | CACCUGCAGAAGAUGCCAGA | SEQ ID NO: 481 |
| mg11-CISH-Exon4-CAG2 | CAGCCUUGUGCAGCACUAUG | SEQ ID NO: 482 |
| hg-mg1-U1-CISH | UCCAGGGACGGGGCCCACAG | SEQ ID NO: 483 |
| hg-mg1-U2-CISH | CUUGGGCAGUUCCAGGGACG | SEQ ID NO: 484 |
| hg-mg1-U3-CISH | GACUGGCUUGGGCAGUUCCA | SEQ ID NO: 485 |
| hg-mg2&3-U1-CISH | GCCCCUGUGGGCCCCGUCCC | SEQ ID NO: 486 |
| hg-mg2&3-U2-CISH | GACUGGGCAGCGGCCCCUGU | SEQ ID NO: 487 |
| hg-mg2&3-U3-CISH | CUGUGGAGCGGACUGGGCAG | SEQ ID NO: 488 |
| hg-mg4-U1-CISH | UGCUGGGGCCUUCCUCGAGG | SEQ ID NO: 489 |
| hg-mg4-U2-CISH | GCCUGCUGGGGCCUUCCUCG | SEQ ID NO: 490 |
| hg-mg4-U3-CISH | CAUGCAGCCCUUGCCUGCUG | SEQ ID NO: 491 |
| hg-mg5&6-U1-CISH | CCACCAGACUACUCAGGAAA | SEQ ID NO: 492 |
| hg-mg5&6-U2-CISH | CUUCUCCCACCAGACUACUC | SEQ ID NO: 493 |
| hg-mg7&8-U1-CISH | CAGGUGUUGUCGGGCCUCGC | SEQ ID NO: 494 |
| hg-mg7&8&9-U2-CISH | CAUCUUCUGCAGGUGUUGUCG | SEQ ID NO: 495 |
| hg-mg7&8&9-U3-CISH | UGCCUUCUGGCAUCUUCUGC | SEQ ID NO: 496 |
| hg-mg9-U1-CISH | CGUACUAAGAACGUGCCUUC | SEQ ID NO: 497 |
| hg-mg10-U1-CISH | CUGGUAUUGGGGUUCCAUUA | SEQ ID NO: 498 |
| hg-mg10-U2-CISH | ACUUGCCUAGGCUGGUAUUG | SEQ ID NO: 499 |
| hg-mg10-U3-CISH | CCCUCUGCACUUGCCUAGGC | SEQ ID NO: 500 |
| hg-mg11-U1-CISH | CUUGUCCAGGCCACGCAUCC | SEQ ID NO: 501 |
| hg-mg11-U2-CISH | UGGACUCCAACUGCUUGUCC | SEQ ID NO: 502 |
| hg-mg11-U3-CISH | UGCCGACUCCAGCUUCCGUC | SEQ ID NO: 503 |
| mg1-TGFBR2-Exon4-CAG | AGCAGAAGCUGAGUUCAACC | SEQ ID NO: 504 |
| mg2-TGFBR2-Exon4-CAG | UUCAGAGCAGUUUGAGACAG | SEQ ID NO: 505 |
| mg3-TGFBR2-Exon4-CAG | ACUCCAGUUCCUGACGGCUG | SEQ ID NO: 506 |
| mg4-TGFBR2-Exon4-CAG | ACCUACAGGAGUACCUGACG | SEQ ID NO: 507 |
| mg5-TGFBR2-Exon4-GT | UUACCUGCCCACUGUUAGCC | SEQ ID NO: 508 |
| mg6-TGFBR2-Exon5-TGG | UUCCCAGAGCACCAGAGCCA | SEQ ID NO: 509 |
| mg7-TGFBR2-Exon6-TGG | AGCCAGAAGCUGGGAAUUUC | SEQ ID NO: 510 |
| hg-mg1-U1-TGFBR2 | AUCAGCCUCCUGCCACCACU | SEQ ID NO: 511 |
| hg-mg1-U2-TGFBR2 | CAUCAGCCUCCUGCCACCAC | SEQ ID NO: 512 |
| hg-mg2-U1-TGFBR2 | UCGCUUUGCUGAGGUCUAUA | SEQ ID NO: 513 |
| hg-mg2-U2-TGFBR2 | GGGGAAAGGUCGCUUUGCUG | SEQ ID NO: 514 |
| hg-mg2-U3-TGFBR2 | CUGGACACCCUGGUGGGGAA | SEQ ID NO: 515 |
| hg-mg2-U4-TGFBR2 | UUGAGCUGGACACCCUGGUG | SEQ ID NO: 516 |
| hg-mg3-U1-TGFBR2 | UGCCUCUUGGAAGACAGAGA | SEQ ID NO: 517 |
| hg-mg3-U2-TGFBR2 | CCUAUGAGGAGUAUGCCUCU | SEQ ID NO: 518 |
| hg-mg4-U1-TGFBR2 | CGGAGUUGGGGAAACAAUAC | SEQ ID NO: 519 |
| hg-mg4-U2-TGFBR2 | AGGAGCGGAAGACGGAGUUG | SEQ ID NO: 520 |
| hg-mg4-U3-TGFBR2 | GAGGAGCGGAAGACGGAGUU | SEQ ID NO: 521 |
| hg-mg4-U4-TGFBR2 | UGAGGAGCGGAAGACGGAGU | SEQ ID NO: 522 |
| hg-mg5-U1-TGFBR2 | AGGCCAGGCUCAAGGUAAAG | SEQ ID NO: 523 |
| hg-mg5-U2-TGFBR2 | GUAGGGUGAGGCCAGGCUCA | SEQ ID NO: 524 |
| hg-mg5-U3-TGFBR2 | UCAAGAGGUAGGGUGAGGCC | SEQ ID NO: 525 |
| hg-mg5-U4-TGFBR2 | AUGGAUCAAGAGGUAGGGUG | SEQ ID NO: 526 |
| hg-mg6-U1-TGFBR2 | UUACCACUACACAAUGAUGC | SEQ ID NO: 527 |
| hg-mg7-U1-TGFBR2 | CUGCCACCUAAGAGGCAACU | SEQ ID NO: 528 |
| hg-mg7-U2-TGFBR2 | AGAAUUCUCUGCCACCUAAG | SEQ ID NO: 529 |
| mg1-FAS-Exon1-TGG | GAGGGUCCAGAUGCCCAGCA | SEQ ID NO: 530 |
| mg2-FAS-Exon1-GT | AGGGCUCACCAGAGGUAGGA | SEQ ID NO: 531 |
| mg3-FAS-Exon2-CAG | GUUGAGACUCAGAACUUGGA | SEQ ID NO: 532 |
| mg4-FAS-Exon2-GT | UACCUGGAGGACAGGGCUUA | SEQ ID NO: 533 |
| mg5-FAS-Exon3-GT | UUCACCUGCCCAAGGAAAAA | SEQ ID NO: 534 |
| mg6-FAS-Exon4-AG | CUAAGCCUAGAAAAUCAGUU | SEQ ID NO: 535 |
| mg7-FAS-Exon5-AG | ACAUCUAGAAAAAAAAAUAC | SEQ ID NO: 536 |
| mg8-FAS-Exon5-GT | AUUACCUUCCUCUUUGCACU | SEQ ID NO: 537 |
| mg9-FAS-Exon6-AG | GAUCCUGUAGGUUGGAACAU | SEQ ID NO: 538 |
| mg10-FAS-Exon6-TGG | AAGCCACCCCAAGUUAGAUC | SEQ ID NO: 539 |
| mg11-FAS-Exon6-TGG | CCCCAAACAAUUAGUGGAAU | SEQ ID NO: 540 |
| mg12-FAS-Exon6-TGG&GT | AACUUACCCCAAACAAUUAG | SEQ ID NO: 541 |
| hg-mg1-U1-FAS | GCCUCCACCCGGGCAGGAGA | SEQ ID NO: 542 |
| hg-mg1-U2-FAS | GGGUAAGCCUCCACCCGGGC | SEQ ID NO: 543 |
| hg-mg1-U3-FAS | GACGGGGUAAGCCUCCACCC | SEQ ID NO: 544 |
| hg-mg2-U1-FAS | CCUAUCCCCGGGACUAAGAC | SEQ ID NO: 545 |
| hg-mg2-U2-FAS | GCCCCACUUUGCCUAUCCCC | SEQ ID NO: 546 |
| hg-mg3-U1-FAS | AGUGACUGACAUCAACUCCA | SEQ ID NO: 547 |
| hg-mg3-U2-FAS | GUGACUGACAUCAACUCCAA | SEQ ID NO: 548 |
| hg-mg3-U3-FAS | UGACAUCAACUCCAAGGGAU | SEQ ID NO: 549 |
| hg-mg4-U1-FAS | GUGACUUUCACUGUAAUCUC | SEQ ID NO: 550 |
| hg-mg5-U1-FAS | UCUUGGCAGGGCACGCAGUC | SEQ ID NO: 551 |
| hg-mg5-U2-FAS | UACUCCUUCCCUUCUUGGCA | SEQ ID NO: 552 |
| hg-mg5-U3-FAS | CUGUGUACUCCUUCCCUUCU | SEQ ID NO: 553 |
| hg-mg6-U1-FAS | GCACUUGGUAUUCUGGGUCC | SEQ ID NO: 554 |
| hg-mg6-U2-FAS | UACAUCUGCACUUGGUAUUC | SEQ ID NO: 555 |
| hg-mg6-U3-FAS | GUUUGGUUUACAUCUGCACU | SEQ ID NO: 556 |
| hg-mg7-U1-FAS | CUCUUUGCACUUGGUGUUGC | SEQ ID NO: 557 |
| hg-mg7-U2-FAS | AUUACCUUCCUCUUUGCACU | SEQ ID NO: 558 |
| hg-mg8-U1-FAS | UUUCCAUGGGGUUGGGGGAA | SEQ ID NO: 559 |
| hg-mg8-U2-FAS | UCACAUCUUUCCAUGGGGUU | SEQ ID NO: 560 |
| hg-mg8-U3-FAS | UUUUUCUUCACAUCUUUCCA | SEQ ID NO: 561 |
| hg-mg9&10-U1-FAS | CCCCAAACAAUUAGUGGAAU | SEQ ID NO: 562 |
| hg-mg9&10-U2-FAS | AACUUACCCCAAACAAUUAG | SEQ ID NO: 563 |
| hg-mg11&12-U1-FAS | GCUUUUUAUAGGUAAGAAUG | SEQ ID NO: 564 |
| mg1-CD7-Exon1-CAA | GCCCAAGGUAAGAGCUUCCC | SEQ ID NO: 565 |
| mg2-CD7-Exon1-GT | GCUCUUACCUUGGGCAGCCA | SEQ ID NO: 566 |
| mg3-CD7-Exon2-AG | UGCACCUCUGGGGAGGACCU | SEQ ID NO: 567 |
| mg4-CD7-Exon2-CAA | CCAAGACAUCAUUUACUACG | SEQ ID NO: 568 |
| mg5-CD7-Exon2-CAG | CGCCUGCAGCUGUCGGACAC | SEQ ID NO: 569 |
| mg6-CD7-Exon2-CAG | CACCUGCCAGGCCAUCACGG | SEQ ID NO: 570 |
| mg7-CD7-Exon2-GT | CCUACCUGUCACCAGGACCA | SEQ ID NO: 571 |
| mg8-CD7-Exon3-CAA | CCAAGGAUGGCACAGAUGCU | SEQ ID NO: 572 |
| mg9-CD7-Exon3-CAG | CAGAGGAACAGUCCCAAGGA | SEQ ID NO: 573 |
| mg10-CD7-Exon3-AG | CCUCUGAGAAGGAAAAAAGA | SEQ ID NO: 574 |
| mg11-CD7-Exon4-CAG | ACCAGUACCAGUGACCCAGU | SEQ ID NO: 575 |
| hg-mg1-U1-CD7 | CUGCUUCUGGCGCUGGCUCG | SEQ ID NO: 576 |
| hg-mg1-U2-CD7 | GCUGCCCCUGCUUCUGGCGC | SEQ ID NO: 577 |
| hg-mg1-U3-CD7 | CCUGCUGCUGCCCCUGCUUC | SEQ ID NO: 578 |
| hg-mg1-U4-CD7 | GGAACAUGGCCGGGCCUCCG | SEQ ID NO: 579 |
| hg-mg2-U1-CD7 | CAGGGAGAGCUCUGGAGCUG | SEQ ID NO: 580 |
| hg-mg2-U2-CD7 | UCAUGGGGCAGGGAGAGCUC | SEQ ID NO: 581 |
| hg-mg2-U3-CD7 | GGGCUCUGAGCUCAUGGGGC | SEQ ID NO: 582 |
| hg-mg2-U4-CD7 | GACUGGGGGCUCUGAGCUCA | SEQ ID NO: 583 |
| hg-mg3-U1-CD7 | UGUUGACGGAGGCUCCCACG | SEQ ID NO: 584 |
| hg-mg3-U2-CD7 | GAUGUUGACGGAGGCUCCCA | SEQ ID NO: 585 |
| hg-mg3-U3-CD7 | GGAGCAGGUGAUGUUGACGG | SEQ ID NO: 586 |
| hg-mg3-U4-CD7 | GGUGGAGCAGGUGAUGUUGA | SEQ ID NO: 587 |
| hg-mg4-U1-CD7 | CCACCAGCGGGGGCCUGCGU | SEQ ID NO: 588 |
| hg-mg4-U2-CD7 | AUCACCUGCUCCACCAGCGG | SEQ ID NO: 589 |
| hg-mg4-U3-CD7 | AACAUCACCUGCUCCACCAG | SEQ ID NO: 590 |
| hg-mg5&6-U1-CD7 | CAUCGACUUCUCAGGGUCCC | SEQ ID NO: 591 |
| hg-mg5-U2-CD7 | GGGGCCGCAUCGACUUCUCA | SEQ ID NO: 592 |
| hg-mg5-U3-CD7 | ACUACGGACAGACGGUUCCG | SEQ ID NO: 593 |
| hg-mg6-U2-CD7 | CAUGCACCGCCUGCAGCUGU | SEQ ID NO: 594 |
| hg-mg7-U1-CD7 | AGCAGCUGGGGUUGGGAGGG | SEQ ID NO: 595 |
| hg-mg7-U2-CD7 | GGCCAGCAGCUGGGGUUGGG | SEQ ID NO: 596 |
| hg-mg7-U3-CD7 | GCCUGGCCAGCAGCUGGGGU | SEQ ID NO: 597 |
| hg-mg7-U4-CD7 | CAGAGCCUGGCCAGCAGCUG | SEQ ID NO: 598 |
| hg-mg8&9-U1-CD7 | GCUCUAACAGGUUCUCCACC | SEQ ID NO: 599 |
| hg-mg8&9-U2-CD7 | CAAACCCCGGGGGCUCUAAC | SEQ ID NO: 600 |
| hg-mg9-U3-CD7 | CCCCGAAUCCCAAACCCCGG | SEQ ID NO: 601 |
| hg-mg10-U1-CD7 | GAGGGCAGAGGCCCUUGGUG | SEQ ID NO: 602 |
| hg-mg10-U2-CD7 | GCAGGGAGGGCAGAGGCCCU | SEQ ID NO: 603 |
| hg-mg10-U3-CD7 | CGGUGGGGCAGGGAGGGCAG | SEQ ID NO: 604 |
| hg-mg10-U4-CD7 | GCCUGUCGGUGGGGCAGGGA | SEQ ID NO: 605 |
| hg-mg11-U1-CD7 | GGCGGCAUGUGUGGUGUACG | SEQ ID NO: 606 |
| hg-mg11-U2-CD7 | UAAGAAUUCGGCGGCAUGUG | SEQ ID NO: 607 |
| hg-mg11-U3-CD7 | GUGGCGGGAUAAGAAUUCGG | SEQ ID NO: 608 |
| mg1-CBLB-Exon1-AG | GCCAUCUGGGGCUGAAAGGC | SEQ ID NO: |
| | | 609 |
| mg2-CBLB-Exon1-GT1 | GCUAACCAAUAAGGGGUGGC | SEQ ID NO: |
| | | 610 |
| mg3-CBLB-Exon1-GT2 | CACCUUUCUUUAAAUCAACG | SEQ ID NO: |
| | | 611 |
| mg4-CBLB-Exon2-CAA | AGCAAGCUGCCGCAGAUCGC | SEQ ID NO: |
| | | 612 |
| mg5-CBLB-Exon2-TGG | GAGCUUCCAAGUCUUCUCCA | SEQ ID NO: |
| | | 613 |
| mg6-CBLB-Exon5-CAG | CCACCAGAUUAGCUCUGGCC | SEQ ID NO: |
| | | 614 |
| mg7-CBLB-Exon5-GT | UAAACUUACCUGAAACAGCC | SEQ ID NO: |
| | | 615 |
| mg8-CBLB-Exon7-CAA | UUAUUUCAAGCCCUGAUUGA | SEQ ID NO: |
| | | 616 |
| mg9-CBLB-Exon9-GT | UUACCUGUGUAACUUUUAUA | SEQ ID NO: |
| | | 617 |
| mg10-CBLB-Exon10-GT | AUUAUACCUGCCAUGCCGUA | SEQ ID NO: |
| | | 618 |
| hg-mg1-U1-CBLB | ACCAAUAAGGGGUGGCAGGU | SEQ ID NO: |
| | | 619 |
| hg-mg1-U2-CBLB | ACGAAGGAGCUAACCAAUAA | SEQ ID NO: |
| | | 620 |
| hg-mg2-U1-CBLB | CGGGAAAGGAAAUGGACGAA | SEQ ID NO: |
| | | 621 |
| hg-mg2-U2-CBLB | AACGAAAGUGGAAACGGGAA | SEQ ID NO: |
| | | 622 |
| hg-mg2-U3-CBLB | GACUCUCCCGGGAACGAAAG | SEQ ID NO: |
| | | 623 |
| hg-mg3-U1-CBLB | CUGUUGUCAACAAAAGCGCA | SEQ ID NO: |
| | | 624 |
| hg-mg4-U1-CBLB | GGGUAUUAUUGAUGCUAUUC | SEQ ID NO: |
| | | 625 |
| hg-mg4-U2-CBLB | CCCCGAAAAGGUCGAAUUUU | SEQ ID NO: |
| | | 626 |
| hg-mg5-U1-CBLB | AAGUGAAUAGUGUUUCGCAC | SEQ ID NO: |
| | | 627 |
| hg-mg6-U1-CBLB | UGCACAGAACUAUCGUACCA | SEQ ID NO: |
| | | 628 |
| hg-mg7-U1-CBLB | AGGGAGAGAGAAGAAGGGAA | SEQ ID NO: |
| | | 629 |
| hg-mg7-U2-CBLB | AAGACAGGGAGAGAGAAGAA | SEQ ID NO: |
| | | 630 |
| hg-mg7-U3-CBLB | AGAAACAGAGAGAAAAGACA | SEQ ID NO: |
| | | 631 |
| hg-mg8-U1-CBLB | UUGGCUAUGUGACUGGGGAU | SEQ ID NO: |
| | | 632 |
| hg-mg8-U2-CBLB | GGGCCAUUGGCUAUGUGACU | SEQ ID NO: |
| | | 633 |
| hg-mg9-U1-CBLB | UAUUUAUUUCAAGGGCAUUA | SEQ ID NO: |
| | | 634 |
| hg-mg9-U2-CBLB | GAGAGUCUUAUUUAUUUCAA | SEQ ID NO: |
| | | 635 |
| hg-mg10-U1-CBLB | CUGAAAAGAGAAAUAUUAGU | SEQ ID NO: |
| | | 636 |
| mg1-KLRC1-Exon1-CAG1- | AGGCAGCAACGAAAACCUAA | SEQ ID NO: |
| CAA1 | | 637 |
| mg2-KLRC1-Exon1-CAG2 | UGAACAGGAAAUAACCUAUG | SEQ ID NO: |
| | | 638 |
| mg3-KLRC1-Exon1-CAA2 | AAACCUUCAAAAAGCUUCUC | SEQ ID NO: |
| | | 639 |
| mg4-KLRC1-Exon1-CAG3 | AAGCUUCUCAGGAUUUUCAA | SEQ ID NO: |
| | | 640 |
| mg5-KLRC1-Exon2-AG | UAAAUCUGCAGGGAGAGAAA | SEQ ID NO: |
| | | 641 |
| hg-mg1-U1-KLRC1 | ACUGCAGAGAUGGAUAACCA | SEQ ID NO: |
| | | 642 |
| hg-mg1-U2-KLRC1 | GACAUCACACACUGCAGAGA | SEQ ID NO: |
| | | 643 |
| hg-mg2-U1-KLRC1 | AGGCAGCAACGAAAACCUAA | SEQ ID NO: |
| | | 644 |
| hg-mg2-U2-KLRC1 | AUCUGCCCCCAAACCCAAAG | SEQ ID NO: |
| | | 645 |
| hg-mg3-U1-KLRC1 | AGGCAGCAACGAAAACCUAA | SEQ ID NO: |
| | | 646 |
| hg-mg3-U2-KLRC1 | CUCCAUUUUAGCAACUGAAC | SEQ ID NO: |
| | | 647 |
| hg-mg4-U1-KLRC1 | UGAACAGGAAAUAACCUAUG | SEQ ID NO: |
| | | 648 |
| hg-mg4-U2-KLRC1 | CUCCAUUUUAGCAACUGAAC | SEQ ID NO: |
| | | 649 |
| hg-mg5-U1-KLRC1 | AGAUAAGACAGAUAAUUCCC | SEQ ID NO: |
| | | 650 |
| mg1-CD38-Exon1-CAG1- | CGCCAGCAGUGGAGCGGUCC | SEQ ID NO: |
| CAG2 | | 651 |
| mg2-CD38-Exon1-TGG | CUCCACUGCUGGCGCCACCU | SEQ ID NO: |
| | | 652 |
| mg3-CD38-Exon2-CAG | CUAUCAGCCACUAAUGAAGU | SEQ ID NO: |
| | | 653 |
| mg4-CD38-Exon2-GT | AAUUACCUUGUUGCAAGGUA | SEQ ID NO: |
| | | 654 |
| mg5-CD38-Exon3-TGG | CUGCUCCAAAGAAGAAUCUA | SEQ ID NO: |
| | | 655 |
| mg6-CD38-Exon3-CAG | AUCAGUUCACACAGGUCCAG | SEQ ID NO: |
| | | 656 |
| mg7-CD38-Exon4-TGG | UUUUCCAGAAUACUGAAACA | SEQ ID NO: |
| | | 657 |
| mg8-CD38-Exon6-CAG | AGGUUCAGACACUAGAGGCC | SEQ ID NO: |
| | | 658 |
| mg9-CD38-Exon7-GT | UUACCUGUAGAUAUUCUUGC | SEQ ID NO: |
| | | 659 |
| hg-mg1-U1-CD38 | GAUCCUCGUCGUGGUGCUCG | SEQ ID NO: |
| | | 660 |
| hg-mg1-U2-CD38 | CCUGGUCCUGAUCCUCGUCG | SEQ ID NO: |
| | | 661 |
| hg-mg1-U3-CD38 | CUGUCUUGGCGUCAGUAUCC | SEQ ID NO: |
| | | 662 |
| hg-mg2-U1-CD38 | CAUCGCGCCAGGACGGUCUC | SEQ ID NO: |
| | | 663 |
| hg-mg2-U2-CD38 | CUUGACGCAUCGCGCCAGGA | SEQ ID NO: |
| | | 664 |
| hg-mg2-U3-CD38 | UGUACUUGACGCAUCGCGCC | SEQ ID NO: |
| | | 665 |
| hg-mg3-U1-CD38 | AGUGUAUGGGAUGCUUUCAA | SEQ ID NO: |
| | | 666 |
| hg-mg4-U1-CD38 | GACCUAUGAAUUGUUACCAU | SEQ ID NO: |
| | | 667 |
| hg-mg5-U1-CD38 | GCUGGACCUGUGUGAACUGA | SEQ ID NO: |
| | | 668 |
| hg-mg5-U2-CD38 | CCAGGGUGAACAUGUCCCGC | SEQ ID NO: |
| | | 669 |
| hg-mg5-U3-CD38 | GCCUAGCAGCGUGUCCUCCA | SEQ ID NO: |
| | | 670 |
| hg-mg6-U1-CD38 | UUCUUCCUUAGAUUCUUCUU | SEQ ID NO: |
| | | 671 |
| hg-mg7-U1-CD38 | AGCUCUAGAAUUUCACUACU | SEQ ID NO: |
| | | 672 |
| hg-mg7-U2-CD38 | GAGUAACGUCUUAAGUUCUG | SEQ ID NO: |
| | | 673 |
| hg-mg8-U1-CD38 | UUUUAGCACUUUUGGGAGUG | SEQ ID NO: |
| | | 674 |
| hg-mg8-U2-CD38 | UUCUUCUAUUUUAGCACUUU | SEQ ID NO: |
| | | 675 |
| hg-mg9-U1-CD38 | AGGAAAAAAGGAAAUUCUAC | SEQ ID NO: |
| | | 676 |
| hg-mg9-U2-CD38 | CAGAGGAGGCUAAGGAAAAA | SEQ ID NO: |
| | | 677 |
| hg-mg9-U3-CD38 | UCCAAGCUCAGAGGAGGCUA | SEQ ID NO: |
| | | 678 |

TABLE 36
mgRNAs and hgRNAs

| | | |
|---|---|---|
| TRAC-mg2-126 | mC*mU*mG*GAUAUCUGUGGGACAAGGUU | SEQ ID NO: |
| | UGAGAGCUAGGGCCCUGAAGAAGGGCCCUA | 679 |
| | GCAAGUUCAAAUAAGGCUAGUCCGUUAUCA | |
| | ACUUGGGCCCUGAAGAAGGGCCCAAGUGGC | |
| | ACCGAGUCGG*mU*mG*mC | |
| TRAC-mg2-hg1- | mA*mG*mA*GUCUCUCAGCUGGUACAGUU | SEQ ID NO: |
| 126 | UGAGAGCUAGGCCAACAUGAGGAUCACCCA | 680 |
| | UGUCUGCAGGGCCUAGCAAGUUCAAAUAAG | |
| | GCUAGUCCGUUAUCAACUUGAAAAAGUGGC | |
| | ACCGAGUCGG*mU*mG*mC | |
| TRAC-mg2-hg1- | mA*mG*mC*UGGUACAGUUUGAGAGCUAGG | SEQ ID NO: |
| 116 | CCAACAUGAGGAUCACCCAUGUCUGCAGGG | 681 |
| | CCUAGCAAGUUCAAAUAAGGCUAGUCCGUU | |
| | AUCAACUUGAAAAAGUGGCACCGAGUCGG* | |
| | mU*mG*mC | |
| TRAC-mg4-126 | mU*mU*mC*GUAUCUGUAAAACCAAGGUUU | SEQ ID NO: |
| | GAGAGCUAGGGCCCUGAAGAAGGGCCCUAG | 682 |
| | CAAGUUCAAAUAAGGCUAGUCCGUUAUCAA | |
| | CUUGGGCCCUGAAGAAGGGCCCAAGUGGCA | |
| | CCGAGUCGG*mU*mG*mC | |
| TRAC-mg4-111 | mU*mU*mC*GUAUCUGUAAAACCAAGGUUU | SEQ ID NO: |
| | GAGAGCUAGGGCCCUGAAGAAGGGCCCUAG | 683 |
| | CAAGUUCAAAUAAGGCUAGUCCGUUAUCAA | |
| | CUUGAAAAAGUGGCACCGAGUCGG*mU*mG* | |
| | mC | |
| TRAC-mg4-hg2- | mC*mC*mA*CUUUCAGGAGGAGGAUUGUU | SEQ ID NO: |
| 126 | UGAGAGCUAGGCCAACAUGAGGAUCACCCA | 684 |
| | UGUCUGCAGGGCCUAGCAAGUUCAAAUAAG | |
| | GCUAGUCCGUUAUCAACUUGAAAAAGUGGC | |
| | ACCGAGUCGG*mU*mG*mC | |
| TRAC-mg4-hg2- | mG*mA*mG*GAGGAUUGUUUGAGAGCUAGG | SEQ ID NO: |
| 116 | CCAACAUGAGGAUCACCCAUGUCUGCAGGG | 685 |
| | CCUAGCAAGUUCAAAUAAGGCUAGUCCGUU | |
| | AUCAACUUGAAAAAGUGGCACCGAGUCGG* | |
| | mU*mG*mC | |
| CD52-mg2-126 | mC*mU*mC*UUACCUGUACCAUAACCGUUU | SEQ ID NO: |
| | GAGAGCUAGGGCCCUGAAGAAGGGCCCUAG | 686 |
| | CAAGUUCAAAUAAGGCUAGUCCGUUAUCAA | |
| | CUUGGGCCCUGAAGAAGGGCCCAAGUGGCA | |
| | CCGAGUCGG*mU*mG*mC | |
| CD52-mg2-111 | mC*mU*mC*UUACCUGUACCAUAACCGUUU | SEQ ID NO: |
| | GAGAGCUAGGGCCCUGAAGAAGGGCCCUAG | 687 |
| | CAAGUUCAAAUAAGGCUAGUCCGUUAUCAA | |
| | CUUGAAAAAGUGGCACCGAGUCGG*mU*mG* | |
| | mC | |
| CD52-mg2-hg3- | mC*mA*mA*GCAACUUGUUUGAGAGCUAGG | SEQ ID NO: |
| 116 | CCAACAUGAGGAUCACCCAUGUCUGCAGGG | 688 |
| | CCUAGCAAGUUCAAAUAAGGCUAGUCCGUU | |
| | AUCAACUUGAAAAAGUGGCACCGAGUCGG* | |
| | mU*mG*mC | |
| CD52-mg3-126 | mG*mU*mA*UCUGUAGGAGGAGAAGUGUU | SEQ ID NO: |
| | UGAGAGCUAGGGCCCUGAAGAAGGGCCCUA | 689 |
| | GCAAGUUCAAAUAAGGCUAGUCCGUUAUCA | |
| | ACUUGGGCCCUGAAGAAGGGCCCAAGUGGC | |
| | ACCGAGUCGG*mU*mG*mC | |
| CD52-mg3-hg2- | mG*mA*mG*GGGCUGCGUUUGAGAGCUAG | SEQ ID NO: |
| 116 | GCCAACAUGAGGAUCACCCAUGUCUGCAGG | 690 |
| | GCCUAGCAAGUUCAAAUAAGGCUAGUCCGU | |
| | UAUCAACUUGAAAAAGUGGCACCGAGUCGG | |
| | *mU*mG*mC | |
| PD1-mg6-111 | mG*mG*mA*GUCUGAGAGAUGGAGAGGUU | SEQ ID NO: |
| | UGAGAGCUAGGGCCCUGAAGAAGGGCCCUA | 691 |
| | GCAAGUUCAAAUAAGGCUAGUCCGUUAUCA | |
| | ACUUGAAAAAGUGGCACCGAGUCGG*mU*m | |
| | G*mC | |
| PD1-mg6-hg2-116 | mG*mC*mU*GGGGAGAGUUUGAGAGCUAG | SEQ ID NO: |
| | GCCAACAUGAGGAUCACCCAUGUCUGCAGG | 692 |
| | GCCUAGCAAGUUCAAAUAAGGCUAGUCCGU | |
| | UAUCAACUUGAAAAAGUGGCACCGAGUCGG | |
| | *mU*mG*mC | |
| PD1-mg7-111 | mG*mG*mG*GUUCCAGGGCCUGUCUGGUU | SEQ ID NO: |
| | UGAGAGCUAGGGCCCUGAAGAAGGGCCCUA | 693 |
| | GCAAGUUCAAAUAAGGCUAGUCCGUUAUCA | |
| | ACUUGAAAAAGUGGCACCGAGUCGG*mU*m | |
| | G*mC | |
| PD1-mg7-hg2-116 | mU*mU*mG*UCCCCUUGUUUGAGAGCUAGG | SEQ ID NO: |
| | CCAACAUGAGGAUCACCCAUGUCUGCAGGG | 694 |
| | CCUAGCAAGUUCAAAUAAGGCUAGUCCGUU | |
| | AUCAACUUGAAAAAGUGGCACCGAGUCGG* | |
| | mU*mG*mC | |
| PD1-mg15-111 | mG*mG*mA*CCCAGACUAGCAGCACCGUU | SEQ ID NO: |
| | UGAGAGCUAGGGCCCUGAAGAAGGGCCCUA | 695 |
| | GCAAGUUCAAAUAAGGCUAGUCCGUUAUCA | |
| | ACUUGAAAAAGUGGCACCGAGUCGG*mU*m | |
| | G*mC | |
| PD1-mg15-hg1-116 | mU*mC*mG*UGCGGCCGUUUGAGAGCUAGG | SEQ ID NO: |
| | CCAACAUGAGGAUCACCCAUGUCUGCAGGG | 696 |
| | CCUAGCAAGUUCAAAUAAGGCUAGUCCGUU | |
| | AUCAACUUGAAAAAGUGGCACCGAGUCGG* | |
| | mU*mG*mC | |
| B2M-mg1-126 | mA*mC*mU*CACGCUGGAUAGCCUCCGUUU | SEQ ID NO: |
| | GAGAGCUAGGGCCCUGAAGAAGGGCCCUAG | 697 |
| | CAAGUUCAAAUAAGGCUAGUCCGUUAUCAA | |
| | CUUGGGCCCUGAAGAAGGGCCCAAGUGGCA | |
| | CCGAGUCGG*mU*mG*mC | |
| B2M-mg1-hg3-116 | mG*mG*mU*GCAGAGCGUUUGAGAGCUAGG | SEQ ID NO: |
| | CCAACAUGAGGAUCACCCAUGUCUGCAGGG | 698 |
| | CCUAGCAAGUUCAAAUAAGGCUAGUCCGUU | |
| | AUCAACUUGAAAAAGUGGCACCGAGUCGG* | |
| | mU*mG*mC | |
| B2M-mg2-126 | mU*mC*mG*AUCUAUGAAAAAGACAGGUUU | SEQ ID NO: |
| | GAGAGCUAGGGCCCUGAAGAAGGGCCCUAG | 699 |
| | CAAGUUCAAAUAAGGCUAGUCCGUUAUCAA | |
| | CUUGGGCCCUGAAGAAGGGCCCAAGUGGCA | |
| | CCGAGUCGG*mU*mG*mC | |
| B2M-mg2-hg1-116 | mC*mA*mU*UUUCUCAGUUUGAGAGCUAGG | SEQ ID NO: |
| | CCAACAUGAGGAUCACCCAUGUCUGCAGGG | 700 |
| | CCUAGCAAGUUCAAAUAAGGCUAGUCCGUU | |
| | AUCAACUUGAAAAAGUGGCACCGAGUCGG* | |
| | mU*mG*mC | |
| CISH-mg2-126 | mG*mU*mC*AUGCAGCCCUUGCCUGCGUU | SEQ ID NO: |
| | UGAGAGCUAGGGCCCUGAAGAAGGGCCCUA | 701 |
| | GCAAGUUCAAAUAAGGCUAGUCCGUUAUCA | |
| | ACUUGGGCCCUGAAGAAGGGCCCAAGUGGC | |
| | ACCGAGUCGG*mU*mG*mC | |
| CISH-mg2-hg2-126 | mG*mA*mC*UGGGCAGCGGCCCCUGUGUU | SEQ ID NO: |
| | UGAGAGCUAGGCCAACAUGAGGAUCACCCA | 702 |
| | UGUCUGCAGGGCCUAGCAAGUUCAAAUAAG | |
| | GCUAGUCCGUUAUCAACUUGAAAAAGUGGC | |
| | ACCGAGUCGG*mU*mG*mC | |
| CISH-mg2-hg2-116 | mC*mG*mG*CCCCUGUGUUUGAGAGCUAGG | SEQ ID NO: |
| | CCAACAUGAGGAUCACCCAUGUCUGCAGGG | 703 |
| | CCUAGCAAGUUCAAAUAAGGCUAGUCCGUU | |
| | AUCAACUUGAAAAAGUGGCACCGAGUCGG* | |
| | mU*mG*mC | |
| CISH-mg3-126 | mC*mA*mU*GCAGCCCUUGCCUGCUGGUU | SEQ ID NO: |
| | UGAGAGCUAGGGCCCUGAAGAAGGGCCCUA | 704 |
| | GCAAGUUCAAAUAAGGCUAGUCCGUUAUCA | |
| | ACUUGGGCCCUGAAGAAGGGCCCAAGUGGC | |
| | ACCGAGUCGG*mU*mG*mC | |
| CISH-mg3-hg3-126 | mC*mU*mG*UGGAGCGGACUGGGCAGGUU | SEQ ID NO: |
| | UGAGAGCUAGGCCAACAUGAGGAUCACCCA | 705 |
| | UGUCUGCAGGGCCUAGCAAGUUCAAAUAAG | |
| | GCUAGUCCGUUAUCAACUUGAAAAAGUGGC | |
| | ACCGAGUCGG*mU*mG*mC | |
| CISH-mg3-hg3-116 | mG*mA*mC*UGGGCAGGUUUGAGAGCUAGG | SEQ ID NO: |
| | CCAACAUGAGGAUCACCCAUGUCUGCAGGG | 706 |
| | CCUAGCAAGUUCAAAUAAGGCUAGUCCGUU | |
| | AUCAACUUGAAAAAGUGGCACCGAGUCGG* | |
| | mU*mG*mC | |
| CISH-mg6-111 | mC*mU*mC*ACCAGAUUCCCGAAGGUGUUU | SEQ ID NO: |
| | GAGAGCUAGGGCCCUGAAGAAGGGCCCUAG | 707 |
| | CAAGUUCAAAUAAGGCUAGUCCGUUAUCAA | |
| | CUUGAAAAAGUGGCACCGAGUCGG*mU*mG* | |
| | mC | |
| CISH-mg6-hg2-116 | mC*mA*mG*ACUACUCGUUUGAGAGCUAGG | SEQ ID NO: |
| | CCAACAUGAGGAUCACCCAUGUCUGCAGGG | 708 |
| | CCUAGCAAGUUCAAAUAAGGCUAGUCCGUU | |
| | AUCAACUUGAAAAAGUGGCACCGAGUCGG* | |
| | mU*mG*mC | |
| TGFBR2-mg3-126 | mA*mC*mU*CCAGUUCCUGACGGCUGGUU | SEQ ID NO: |
| | UGAGAGCUAGGGCCCUGAAGAAGGGCCCUA | 709 |
| | GCAAGUUCAAAUAAGGCUAGUCCGUUAUCA | |
| | ACUUGGGCCCUGAAGAAGGGCCCAAGUGGC | |
| | ACCGAGUCGG*mU*mG*mC | |
| TGFBR2-mg3-hg1- | mU*mG*mC*CUCUUGGAAGACAGAGAGUU | SEQ ID NO: |
| 126 | UGAGAGCUAGGCCAACAUGAGGAUCACCCA | 710 |
| | UGUCUGCAGGGCCUAGCAAGUUCAAAUAAG | |
| | GCUAGUCCGUUAUCAACUUGAAAAAGUGGC | |
| | ACCGAGUCGG*mU*mG*mC | |
| TGFBR2-mg3-hg1- | mA*mA*mG*ACAGAGAGUUUGAGAGCUAGG | SEQ ID NO: |
| 116 | CCAACAUGAGGAUCACCCAUGUCUGCAGGG | 711 |
| | CCUAGCAAGUUCAAAUAAGGCUAGUCCGUU | |
| | AUCAACUUGAAAAAGUGGCACCGAGUCGG* | |
| | mU*mG*mC | |
| TGFBR2-mg4-126 | mA*mC*mC*UACAGGAGUACCUGACGGUU | SEQ ID NO: |
| | UGAGAGCUAGGGCCCUGAAGAAGGGCCCUA | 712 |
| | GCAAGUUCAAAUAAGGCUAGUCCGUUAUCA | |
| | ACUUGGGCCCUGAAGAAGGGCCCAAGUGGC | |
| | ACCGAGUCGG*mU*mG*mC | |
| TGFBR2-mg4-hg1- | mC*mG*mG*AGUUGGGGAAACAAUACGUU | SEQ ID NO: |
| 126 | UGAGAGCUAGGCCAACAUGAGGAUCACCCA | 713 |
| | UGUCUGCAGGGCCUAGCAAGUUCAAAUAAG | |
| | GCUAGUCCGUUAUCAACUUGAAAAAGUGGC | |
| | ACCGAGUCGG*mU*mG*mC | |
| TGFBR2-mg4-hg1- | mG*mA*mA*ACAAUACGUUUGAGAGCUAGG | SEQ ID NO: |
| 116 | CCAACAUGAGGAUCACCCAUGUCUGCAGGG | 714 |
| | CCUAGCAAGUUCAAAUAAGGCUAGUCCGUU | |
| | AUCAACUUGAAAAAGUGGCACCGAGUCGG* | |
| | mU*mG*mC | |
| FAS-mg1-126 | mG*mA*mG*GGUCCAGAUGCCCAGCAGUU | SEQ ID NO: |
| | UGAGAGCUAGGGCCCUGAAGAAGGGCCCUA | 715 |
| | GCAAGUUCAAAUAAGGCUAGUCCGUUAUCA | |
| | ACUUGGGCCCUGAAGAAGGGCCCAAGUGGC | |
| | ACCGAGUCGG*mU*mG*mC | |
| FAS-mg1-hg1-116 | mG*mG*mG*CAGGAGAGUUUGAGAGCUAG | SEQ ID NO: |
| | GCCAACAUGAGGAUCACCCAUGUCUGCAGG | 716 |
| | GCCUAGCAAGUUCAAAUAAGGCUAGUCCGU | |
| | UAUCAACUUGAAAAAGUGGCACCGAGUCGG | |
| | *mU*mG*mC | |
| FAS-mg5-126 | mU*mU*mC*ACCUGCCCAAGGAAAAAGUUU | SEQ ID NO: |
| | GAGAGCUAGGGCCCUGAAGAAGGGCCCUAG | 717 |
| | CAAGUUCAAAUAAGGCUAGUCCGUUAUCAA | |
| | CUUGGGCCCUGAAGAAGGGCCCAAGUGGCA | |
| | CCGAGUCGG*mU*mG*mC | |
| FAS-mg5-hg1-116 | mG*mC*mA*CGCAGUCGUUUGAGAGCUAGG | SEQ ID NO: |
| | CCAACAUGAGGAUCACCCAUGUCUGCAGGG | 718 |
| | CCUAGCAAGUUCAAAUAAGGCUAGUCCGUU | |
| | AUCAACUUGAAAAAGUGGCACCGAGUCGG* | |
| | mU*mG*mC | |
| CD38-mg2-126 | mC*mU*mC*CACUGCUGGCGCCACCUGUUU | SEQ ID NO: |
| | GAGAGCUAGGGCCCUGAAGAAGGGCCCUAG | 719 |
| | CAAGUUCAAAUAAGGCUAGUCCGUUAUCAA | |
| | CUUGGGCCCUGAAGAAGGGCCCAAGUGGCA | |
| | CCGAGUCGG*mU*mG*mC | |
| CD38-mg2-hg2- | mC*mG*mC*GCCAGGAGUUUGAGAGCUAGG | SEQ ID NO: |
| 116 | CCAACAUGAGGAUCACCCAUGUCUGCAGGG | 720 |
| | CCUAGCAAGUUCAAAUAAGGCUAGUCCGUU | |
| | AUCAACUUGAAAAAGUGGCACCGAGUCGG* | |
| | mU*mG*mC | |
| CD38-mg7-126 | mU*mU*mU*UCCAGAAUACUGAAACAGUUU | SEQ ID NO: |
| | GAGAGCUAGGGCCCUGAAGAAGGGCCCUAG | 721 |
| | CAAGUUCAAAUAAGGCUAGUCCGUUAUCAA | |
| | CUUGGGCCCUGAAGAAGGGCCCAAGUGGCA | |
| | CCGAGUCGG*mU*mG*mC | |
| CD38-mg72-hg1- | mU*mU*mU*CACUACUGUUUGAGAGCUAGG | SEQ ID NO: |
| 116 | CCAACAUGAGGAUCACCCAUGUCUGCAGGG | 722 |
| | CCUAGCAAGUUCAAAUAAGGCUAGUCCGUU | |
| | AUCAACUUGAAAAAGUGGCACCGAGUCGG* | |
| | mU*mG*mC | |
| CBLB-mg2-111 | mG*mC*mU*AACCAAUAAGGGGUGGCGUU | SEQ ID NO: |
| | UGAGAGCUAGGGCCCUGAAGAAGGGCCCUA | 723 |
| | GCAAGUUCAAAUAAGGCUAGUCCGUUAUCA | |
| | ACUUGAAAAAGUGGCACCGAGUCGG*mU*m | |
| | G*mC | |
| CBLB-mg2-hg2- | mG*mA*mA*ACGGGAAGUUUGAGAGCUAGG | SEQ ID NO: |
| 116 | CCAACAUGAGGAUCACCCAUGUCUGCAGGG | 724 |
| | CCUAGCAAGUUCAAAUAAGGCUAGUCCGUU | |
| | AUCAACUUGAAAAAGUGGCACCGAGUCGG* | |
| | mU*mG*mC | |
| CBLB-mg10-111 | mA*mU*mU*AUACCUGCCAUGCCGUAGUUU | SEQ ID NO: |
| | GAGAGCUAGGGCCCUGAAGAAGGGCCCUAG | 725 |
| | CAAGUUCAAAUAAGGCUAGUCCGUUAUCAA | |
| | CUUGAAAAAGUGGCACCGAGUCGG*mU*mG* | |
| | mC | |
| CBLB-mg10-hg1- | mA*mA*mA*UAUUAGUGUUUGAGAGCUAGG | SEQ ID NO: |
| 116 | CCAACAUGAGGAUCACCCAUGUCUGCAGGG | 726 |
| | CCUAGCAAGUUCAAAUAAGGCUAGUCCGUU | |
| | AUCAACUUGAAAAAGUGGCACCGAGUCGG* | |
| | mU*mG*mC | |
| KLRC1-mg2-111 | mU*mG*mA*ACAGGAAAUAACCUAUGGUUU | SEQ ID NO: |
| | GAGAGCUAGGGCCCUGAAGAAGGGCCCUAG | 727 |
| | CAAGUUCAAAUAAGGCUAGUCCGUUAUCAA | |
| | CUUGAAAAAGUGGCACCGAGUCGG*mU*mG* | |
| | mC | |
| KLRC1-mg2-hg1- | mG*mA*mA*AACCUAAGUUUGAGAGCUAGG | SEQ ID NO: |
| 116 | CCAACAUGAGGAUCACCCAUGUCUGCAGGG | 728 |
| | CCUAGCAAGUUCAAAUAAGGCUAGUCCGUU | |
| | AUCAACUUGAAAAAGUGGCACCGAGUCGG* | |
| | mU*mG*mC | |
| KLRC1-mg5-111 | mU*mA*mA*AUCUGCAGGGAGAGAAAGUU | SEQ ID NO: |
| | UGAGAGCUAGGGCCCUGAAGAAGGGCCCUA | 729 |
| | GCAAGUUCAAAUAAGGCUAGUCCGUUAUCA | |
| | ACUUGAAAAAGUGGCACCGAGUCGG*mU*m | |
| | G*mC | |
| KLRC1-mg5-hg1- | mG*mA*mU*AAUUCCCGUUUGAGAGCUAGG | SEQ ID NO: |
| 116 | CCAACAUGAGGAUCACCCAUGUCUGCAGGG | 730 |
| | CCUAGCAAGUUCAAAUAAGGCUAGUCCGUU | |
| | AUCAACUUGAAAAAGUGGCACCGAGUCGG* | |
| | mU*mG*mC | |

Spacer sequence is in bold.
“m” refers to 2'-O-methylation of the nucleotide.
“*” refers to 3'-phosphorothioate linkage between the nucleotides.

TABLE 37

| | | |
|---|---|---|
| tBE-V5- | gggggacagatcgcctggagacgccatccacgctgttttgacctccatagaagacaccgggaccgatcca | SEQ |
| mA3 | gcctccgcggccgggaacggtgcattggaacgcggattccccgtgccaagagtgactcaccgtccttgac | ID |
| mRNA | acggccaccatggcctctaacttcacccagtttgtgctggtggacaatggaggaaccggcgatgtgacagt | NO: |
| | ggcaccatccaactttgccaatggcatcgccgagtggatcagctccaactctaggagccaggcctacaagg | 731 |
| | tgacctgcagcgtgcggcagtctagcgcccagaatagaaagtacacaatcaaggtggaggtgccaaagg | |
| | gagcatggcgcagctatctgaacatggagctgaccatccccatcttcgccacaaattccgactgtgagctga | |
| | tcgtgaaggccatgcagggcctgctgaaggatggcaaccccatcccttctgccatcgccgccaatagcgg | |
| | catctactctggaggatctagcggaggcagctctggcagcgagacaccaggaacaagcgagtcagcaac | |
| | accagagagcagtggcggcagcagcggcggcagcagcggcggatccaagcggcccgccgccaccaa | |
| | gaaggccggccaggccaagaagaagaagatgaccaacctgtctgacatcatcgagaaggagacaggca | |
| | agcagctggtcatccaggagagcatcctgatgctgcccgaagaagtcgaagaagtgatcggaaacaagcc | |
| | tgagagcgatatcctggtccataccgcctacgacgagagtaccgacgaaaatgtgatgctgctgacatccg | |
| | acgccccagagtataagccctgggctctggtcatccaggattccaacggagagaacaaaatcaaaatgctg | |
| | tctggaggcagcatgggaccattttgcctgggctgttctcaccggaagtgctatagccccatcagaaacctg | |
| | atcagccaggagacattcaagtttcacttcaagaatctgggctacgccaagggccggaaggataccttcctg | |
| | tgctatgaggtgacaagaaaggactgtgatagccccgtgtccctgcaccacggcgtgtttaagaacaagga | |
| | caatatccacgccgagatctgttttctgtactggttccacgataaggtgctgaaggtgctgtcccccagggag | |
| | gagttcaagatcacctggtatatgtcttggagcccttgcttcgagtgtgccgagcagatcgtgcgctttctgg | |
| | ccacacaccacaacctgagcctggacatctttagctcccggctgtacaacgtgcaggatcctgagacacagc | |
| | agaatctgtgcagactggtgcaggagggagcacaggtggcagcaatggacctgtatgagttcaagaagtg | |
| | ttggaagaagtttgtggataacggcggccggagattccggccgtggaagagactgctgaccaacttccggt | |
| | accaggacagcaagctgcaggagatcctgcgcccttgctatatctccgtgccatctagcgagaatctgtactt | |
| | ccagagcatgtcctctagcaccctgtctaatatctgcctgaccaagggcctgcccgagacaaggttctgggt | |
| | ggagggccggagaatggaccctctgagcgaggaggagttttactcccagttctataaccagagggtgaag | |
| | cacctgtgctactatcaccgcatgaagccttacctgtgctatcagctggagcagttcaatggacaggcacca | |
| | ctgaagggatgcctgctgtccgagaagggcaagcagcacgccgagatcctgtttctggataagatcagatc | |
| | tatggagctgagccaggtgaccatcacatgttacctgacatggtccccatgcccaaactgtgcatggcagct | |
| | ggcagccttcaagagggaccgcccagatctgatcctgcacatctacacctctaggctgtattttcactggaag | |
| | cgccccttccagaagggcctgtgctccctgtggcagtctggcatcctggtggacgtgatggatctgccccag | |
| | tttaccgactgttggacaaacttcgtgaatcctaagcggccattttggccctggaagggcctggagatcatca | |
| | gcaggcgcacacagcggagactgaggcgcatcaaggagtcctggggcctgcaggacctggtgaacgatt | |
| | ttggcaatctgcagctgggaccccctatgagcaagcggcccgccgccaccaagaaggccggccaggcc | |
| | aagaagaagaaggctactaacttcagcctgctgaagcaggctggggacgtggaggagaaccctggacct | |
| | atgggcaacgccagaacaagacggcgggagcgcagagccgagaagcaggcccaatggaaagccgcta | |
| | attccggcgggagcagtggcggatccagtgggtccgaaacccctggcacaagcgaaagcgctacccctg | |
| | agtccagcggagggtcctcaggggaattcatgaaaagcatgtctagcatggtgtctgacacaagctgtacct | |
| | tcccctcctctgatggcatcttttggaagcactggattcagacaaaggacggccagtgtggctcccctctggt | |
| | gtctaccagggatggcttcatcgtgggcatccacagcgcctccaacttcacaaacaccaacaactactttaca | |
| | tccgtgcctaagaacttcatggagctgctgaccaatcaggaggcacagcagtgggtgtctggatggcgcct | |
| | gaacgccgatagcgtgctgtggggcggccacaaggtgtttatggtgaagccagaggagcccttccagcct | |
| | gtgaaggaggccacccagaagcggcccgccgccaccaagaaggccggccaggccaagaagaagaag | |
| | gagggcagaggaagtctgctaacatgcggtgacgtcgaggagaatcctggcccaatgggcgagtccctgt | |
| | ttaagggcccacgggactacaaccccatcagctccacaatctgccacctgaccaatgagagcgatggcca | |
| | caccacatccctgtatggcatcggcttcggccccttcatcatcacaaacaagcacctgtttcggagaaacaat | |
| | ggcaccctgctggtgcagtctctgcacggcgtgttcaaggtgaagaataccacaaccctgcagcagcacct | |
| | gatcgacggaagggatatgatcatcatcagaatgcctaaggacttccccccttttccacagaagctgaagttt | |
| | cgggagccacagagggaggagaggatctgcctggtgacaaccaacttccagacactcgagaagcggcc | |
| | cgccgccaccaagaaggccggccaggccaagaagaagaagtaatgagctccgggtggcatccctgtga | |
| | cccctccccagtgcctctcctggccctggaagttgccactccagtgcccaccagccttgtcctaataaaatta | |
| | agttgcatcaagctgcggccgcaaaaaaaaaaaaaaaaaaaaaaaaaaaaaaaaaaaaaaaaaaaaaaa | |
| | aaaaaaaaaaaaaaaaaaaaaaaaaaaaaaaaaaaaaaaaaaaaaaaaaaaaa | |
| nCas9 | gggGGACAGATCGCCTGGAGACGCCATCCACGCTGTTTTGACCTC | SEQ |
| mRNA | CATAGAAGACACCGGGACCGATCCAGCCTCCGCGGCCGGGAACG | ID |
| | GTGCATTGGAACGCGGATTCCCCGTGCCAAGAGTGACTCACCGT | NO: |
| | CCTTGACACGgccaccatgGCCCCTGCCGCCAAGCGGGTCAAGCTCG | 732 |
| | ACGGTATCCACGGAGTCCCAGCAGCtatggacaagaagtacagcatcggcctggc | |
| | catcggcaccaacagcgtgggctgggccgtgatcaccgacgagtacaaggtgcccagcaagaagttcaa | |
| | ggtgctgggcaacaccgaccggcacagcatcaagaagaacctgatcggcgccctgctgttcgacagcgg | |
| | cgagaccgccgaggccacccggctgaagcggaccgcccggcggcggtacacccggcggaagaaccg | |
| | gatctgctacctgcaggagatcttcagcaacgagatggccaaggtggacgacagcttcttccaccggctgg | |
| | aggagagcttcctggtggaggaggacaagaagcacgagcggcaccccatcttcggcaacatcgtggacg | |
| | aggtggcctaccacgagaagtaccccaccatctaccacctgcggaagaagctggtggacagcaccgaca | |
| | aggccgacctgcggctgatctacctggccctggcccacatgatcaagttccggggccacttcctgatcgag | |
| | ggcgacctgaaccccgacaacagcgacgtggacaagctgttcatccagctggtgcagacctacaaccagc | |
| | tgttcgaggagaaccccatcaacgccagcggcgtggacgccaaggccatcctgagcgcccggctgagca | |
| | agagccggcggctggagaacctgatcgcccagctgcccggcgagaagaagaacggcctgttcggcaac | |
| | ctgatcgccctgagcctgggcctgacccccaacttcaagagcaacttcgacctggccgaggacgccaagc | |
| | tgcagctgagcaaggacacctacgacgacgacctggacaacctgctggcccagatcggcgaccagtacg | |
| | ccgacctgttcctggccgccaagaacctgagcgacgccatcctgctgagcgacatcctgcgggtgaacac | |
| | cgagatcaccaaggcccccctgagcgccagcatgatcaagcggtacgacgagcaccaccaggacctga | |
| | ccctgctgaaggccctggtgcggcagcagctgcccgagaagtacaaggagatcttcttcgaccagagcaa | |
| | gaacggctacgccggctacatcgacggcggcgccagccaggaggagttctacaagttcatcaagcccatc | |
| | ctggagaagatggacggcaccgaggagctgctggtgaagctgaaccgggaggacctgctgcggaagca | |
| | gcggaccttcgacaacggcagcatcccccaccagatccacctgggcgagctgcacgccatcctgcggcg | |
| | gcaggaggacttctaccccttcctgaaggacaaccgggagaagatcgagaagatcctgaccttccggatcc | |
| | cctactacgtgggccccctggcccggggcaacagccggttcgcctggatgacccgaaagagcgaggag | |
| | accatcaccccctggaacttcgaggaggtggtggacaagggcgccagcgcccagagcttcatcgagcgg | |
| | atgaccaacttcgacaagaacctgcccaacgagaaggtgctgcccaagcacagcctgctgtacgagtactt | |
| | caccgtgtacaacgagctgaccaaggtgaagtacgtgaccgagggcatgcggaagcccgccttcctgag | |
| | cggcgagcagaagaaggccatcgtggacctgctgttcaagaccaaccggaaggtgaccgtgaagcagct | |
| | gaaggaggactacttcaagaagatcgagtgcttcgacagcgtggagatcagcggcgtggaggaccggttc | |
| | aacgccagcctgggcacctaccacgacctgctgaagatcatcaaggacaaggacttcctggacaacgagg | |
| | agaacgaggacatcctggaggacatcgtgctgaccctgaccctgttcgaggaccgggagatgatcgagga | |
| | gcggctaaagacctacgcccacctgttcgacgacaaggtgatgaagcagctgaagcggcggcggtacac | |
| | cggctggggccggctgagccggaagctgatcaacggcatccgggacaagcagagcggcaagaccatcc | |
| | tggacttcctcaagagcgacggcttcgccaaccggaacttcatgcagctgatccacgacgacagcctgacc | |
| | ttcaaggaggacatccagaaggcccaggtgagcggccagggcgacagcctgcacgagcacatcgccaa | |
| | cctggccggcagccccgccatcaagaagggcatcctgcagaccgtgaaggtggtggacgagctggtgaa | |
| | ggtgatgggccggcacaagcccgagaacatcgtgatcgagatggcccgggagaaccagaccacccaga | |
| | agggccagaagaacagccgggagcggatgaagcggatcgaggagggcatcaaggagctgggcagcc | |
| | agatcctgaaggagcaccccgtggagaacacccagctgcagaacgagaagctgtacctgtactacctgca | |
| | gaacggccgggacatgtacgtggaccaggagctggacatcaaccggctgagcgactacgacgtggacc | |
| | acatcgtgccccagagcttcctgaaggacgacagcatcgacaacaaggtgctgacccggagcgacaaga | |
| | accggggcaagagcgacaacgtgcccagcgaggaggtggtgaagaagatgaagaactactggcggca | |
| | gctgctgaacgccaagctgatcacccagcggaagttcgacaacctgaccaaggccgagcggggcggcct | |
| | gagcgagctggacaaggccggcttcatcaagcggcagctggtggagacccggcagatcaccaagcacg | |
| | tggcccagatcctggacagccggatgaacaccaagtacgacgagaacgacaagctgatccgggaggtga | |
| | aggtgatcaccctcaagagcaagctggtgagcgacttccggaaggacttccagttctacaaggtgcggga | |
| | gatcaacaactaccaccacgcccacgacgcctacctgaacgccgtggtgggcaccgccctgatcaagaa | |
| | gtaccccaagctggagagcgagttcgtgtacggcgactacaaggtgtacgacgtgcggaagatgatcgcc | |
| | aagagcgagcaggagatcggcaaggccaccgccaagtacttcttctacagcaacatcatgaacttcttcaa | |
| | gaccgagatcaccctggccaacggcgagatccggaagcggcccctgatcgagaccaacggcgagaccg | |
| | gcgagatcgtgtgggacaagggccgggacttcgccaccgtgcggaaggtgctgagcatgccccaggtga | |
| | acatcgtgaaaaagaccgaggtgcagaccggcggcttcagcaaggagagcatcctgcccaagcggaac | |
| | agcgacaagctgatcgcccggaagaaggactgggaccccaagaagtacggcggcttcgacagccccac | |
| | cgtggcctacagcgtgctggtggtggccaaggtggagaagggcaagagcaagaagctcaagagcgtga | |
| | aggagctgctgggcatcaccatcatggagcggagcagcttcgagaagaaccccatcgacttcctggaggc | |
| | caagggctacaaggaggtgaagaaggacctgatcatcaagctgcccaagtacagcctgttcgagctggag | |
| | aacggccggaagcggatgctggccagcgccggcgagctgcagaagggcaacgagctggccctgccca | |
| | gcaagtacgtgaacttcctgtacctggccagccactacgagaagctgaagggcagccccgaggacaacg | |
| | agcagaagcagctgttcgtggagcagcacaagcactacctggacgagatcatcgagcagatcagcgagtt | |
| | cagcaagcgggtgatcctggccgacgccaacctggacaaggtgctgagcgcctacaacaagcaccggg | |
| | acaagcccatccgggagcaggccgagaacatcatccacctgttcaccctgaccaacctgggcgcccccg | |
| | ccgccttcaagtacttcgacaccaccatcgaccggaagcggtacaccagcaccaaggaggtgctggacgc | |
| | caccctgatccaccagagcatcaccggcctgtacgagacccggatcgacctgagccagctgggcggcga | |
| | cagcggcggcagcAAGCGGCCCGCCGCCACCAAGAAGGCCGGCCAGGC | |
| | CAAGAAGAAGAAGtaatgagctcCGGGTGGCATCCCTGTGACCCCTCC | |
| | CCAGTGCCTCTCCTGGCCCTGGAAGTTGCCACTCCAGTGCCCACC | |
| | AGCCTTGTCCTAATAAAATTAAGTTGCATCAAGCTgcggccgcaaaaaaa | |
| | aaaaaaaaaaaaaaaaaaaaaaaaaaaaaaaaaaaaaaaaaaaaaaaaaaaaaaaaaaaaaaaaaaaa | |
| | aaaaaaaaaaaaaaaaaaaaaaaaa | |

### EXAMPLES

### Gene Editing Efficiency Tests

To apply the tBE system to generate stop codons or destroy splicing sites in the target genes (TRAC gene, B2M gene, CD52 gene, PDCD1 gene, CTLA4 gene, TIGIT gene, TIM3 gene, LAG3 gene, CISH gene, TGFBR2 gene, FAS gene, CD7 gene, CBLB gene, KLRC1 gene, and CD38 gene), 15 pairs of mgRNA/hgRNAs that target TRAC gene, 9 pairs of mgRNA/hgRNAs that target CD52 gene, 45 pairs of mgRNA/hgRNAs that target B2M, 47 pairs of mgRNA/hgRNAs that target PDCD1, 23 pairs of mgRNA/hgRNAs that target CTLA4, 47 pairs of mgRNA/hgRNAs that target TIGIT, 41 pairs of mgRNA/hgRNAs that target TIM3, 85 pairs of mgRNA/hgRNAs that target LAG3, 31 pairs of mgRNA/hgRNAs that target CISH, 19 pairs of mgRNA/hgRNAs that target TGFBR2, 26 pairs of mgRNA/hgRNAs that target FAS, 36 pairs of mgRNA/hgRNAs that target CD7, 18 pairs of mgRNA/hgRNAs that target CBLB, 9 pairs of mgRNA/hgRNAs that target KLRC1, and 19 pairs of mgRNA/hgRNAs that target CD38 were designed. tBE systems comprising these respective pairs of guide RNAs were used to induce C-to-T base editing in the codons of CAA (Gin), CAG (Gin), TGG (Trp, C-to-T on the opposite strand) or CGA (Arg) in the target genes to create TAA, TAG, or TGA stop codon. Genomic DNA was extracted 72 hours after transfecting plasmids into cells. The C-to-T editing efficiencies of different mgRNA/hgRNA pairs with tBE at target sites were analyzed. The Sanger sequencing results show that tBE could perform highly efficient base editing to generate stop codons in the target genes.

tBE systems comprising these pairs of guide RNAs were also used to induce G-to-A (C-to-T on the opposite strand) base editing in GT or AG splice site to destroy the GU-AG canonical splicing pattern. The Sanger sequencing results show that tBE also induced high base editing efficiencies at these target sites.

The base editors, the mgRNAs and hgRNAs, and the base editing methods disclosed herein can be applied to perform high-specificity and high-efficiency base editing in the genome of various eukaryotes.

### Plasmid Construction

Primer sets (hg-mg1&2-U1-TRAC_FOR/mg1-TRAC-Exon1-AG1_REV) were used to amplify the fragment hg-mg1&2-U1-TRAC-MS2 (the operator in hgRNA scaffold)-U6 (mgRNA promoter)-mg1-TRAC-Exon1-AG1 using the template pUC57-mgRNA-MS2-U6. The fragment hg-mg1&2-U1-TRAC-MS2-U6-mg1-TRAC-Exon1-AG1 was then ligated into BsmBI-linearized U6-ccdB-boxB-tBE-V5 to generate the vector ptBE-V5-TRAC-E1-AG1-U1. Other combinations with different on-target hgRNA and mgRNA were constructed using the same strategy, respectively.

### Cell Culture and Transfection

293FT cells were maintained in DMEM+10% FBS and regularly tested to exclude mycoplasma contamination. For base editing with transformer BEs, 293FT cells were seeded in a 24-well plate at a density of 1×10⁵ per well and transfected with 250 μl serum-free Opti-MEM containing 2.5 μl LIPOFECTAMINE LTX, 1 μl LIPOFECTAMINE plus, 0.5 μg tBE-V5 expression vector, 0.5 μg pEFS-nSpCas9 or pEFS-nSpCas9-NG expression vector. After 24 h, puromycin was added to the medium at a final concentration of 4 μg ml⁻¹. After another 48 h, the genomic DNA was extracted from the cells using QuickExtractT DNA Extraction Solution for subsequent sequencing analysis. Target genomic sequences were PCR-amplified using high-fidelity DNA polymerase PrimeSTAR HS with primer sets flanking the examined mgRNA target sites.

Base substitution frequency at each target sites was calculated by EditR analysis. See http://baseeditr.com/.

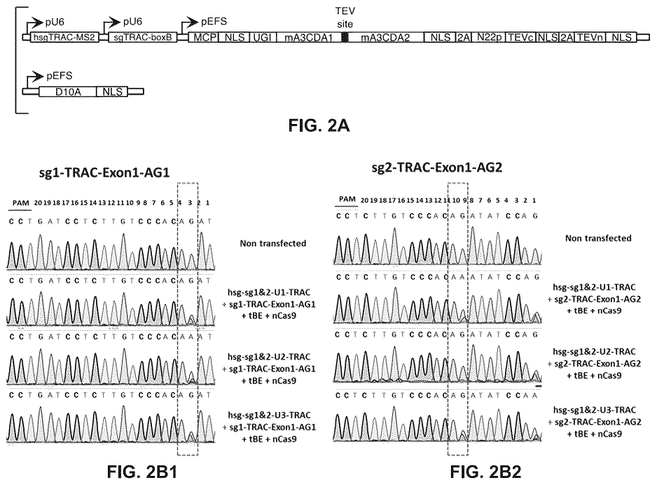

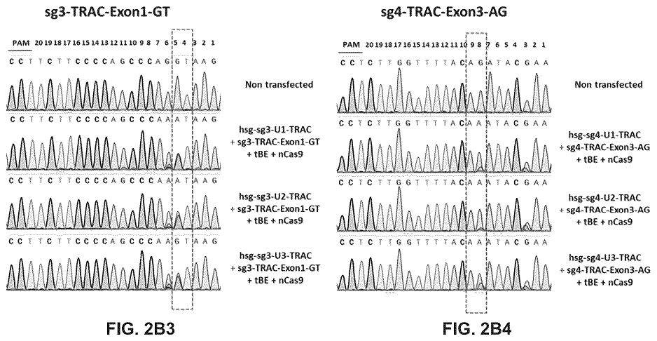

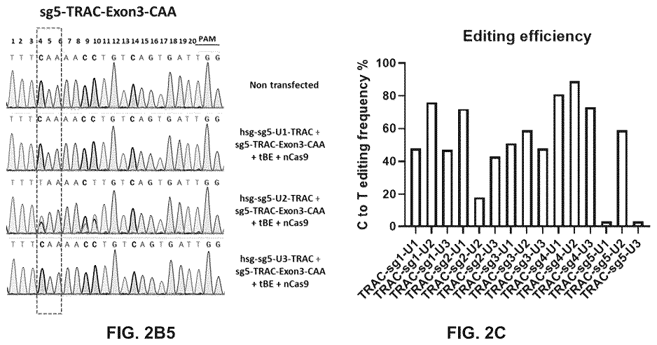

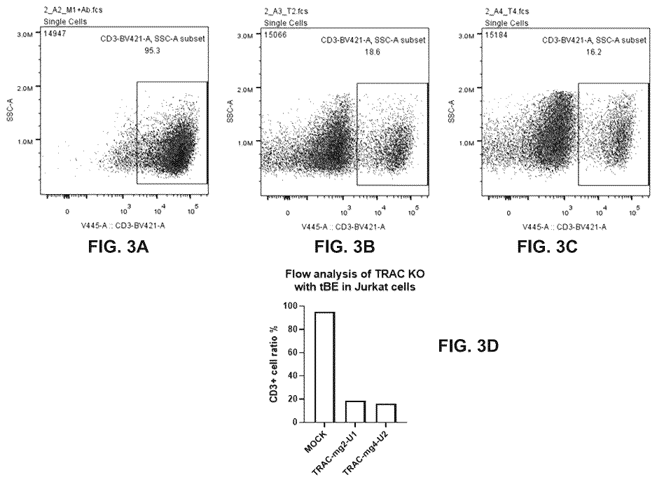

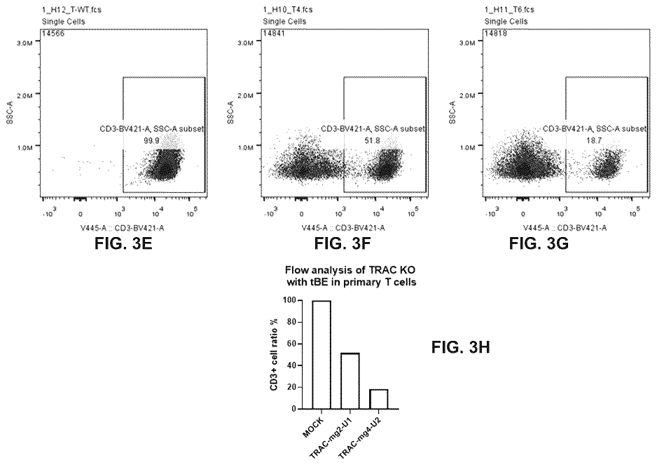

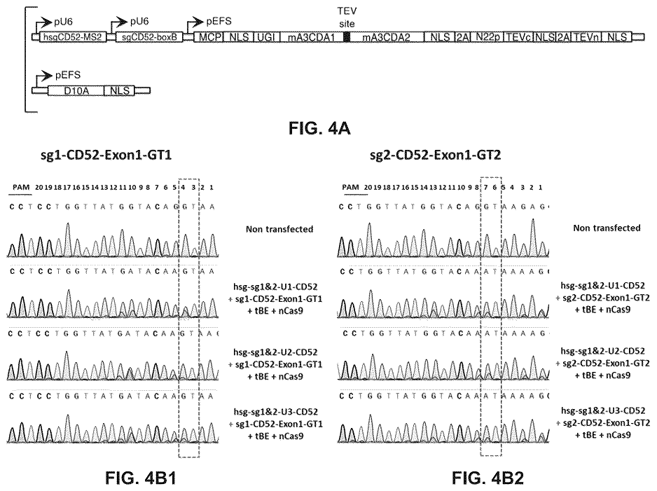

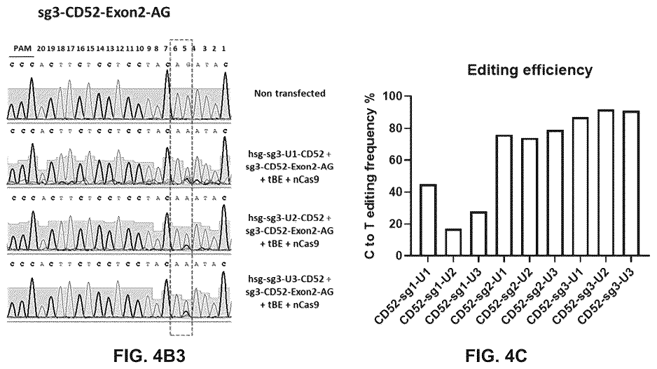

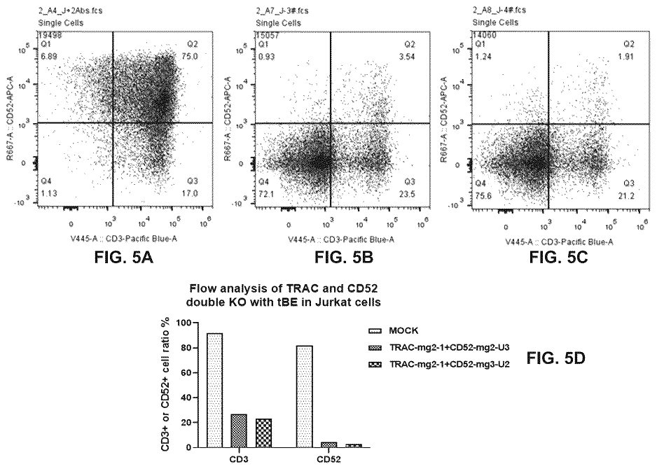

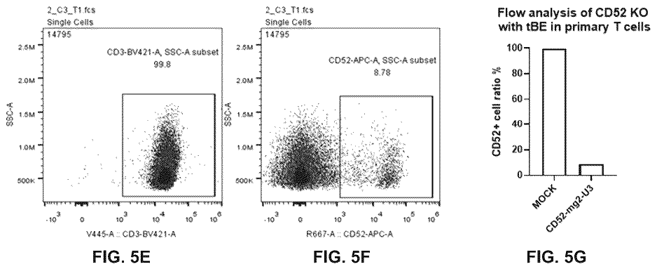

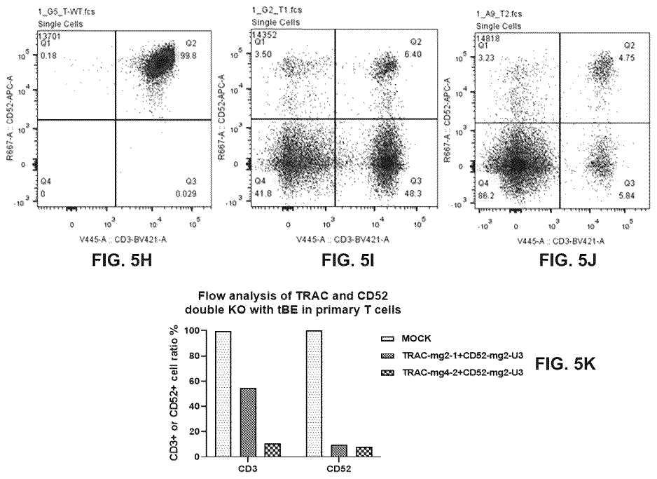

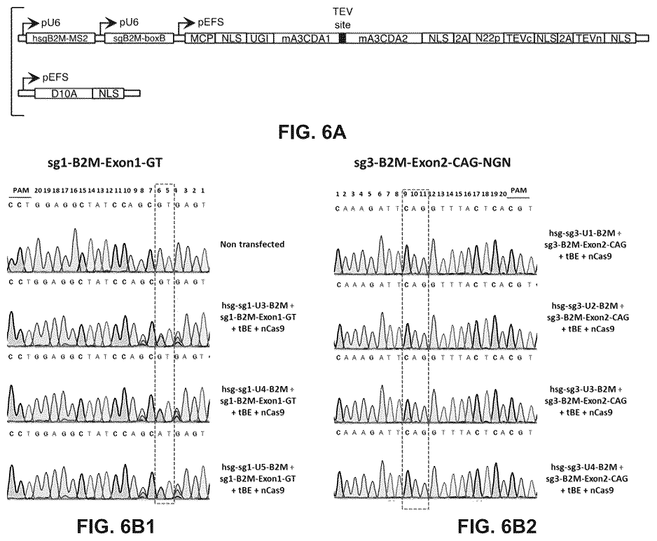

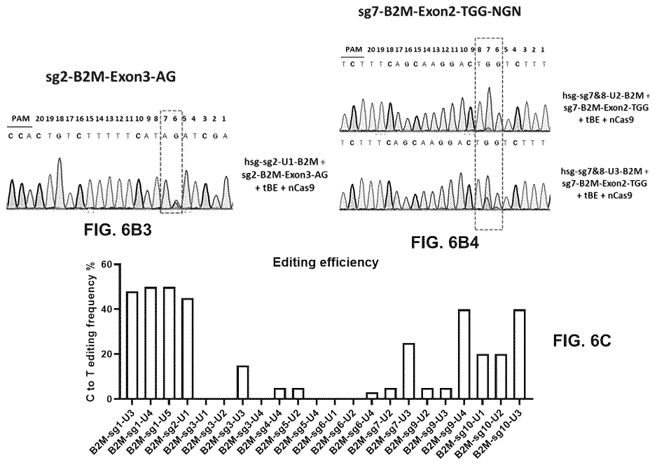

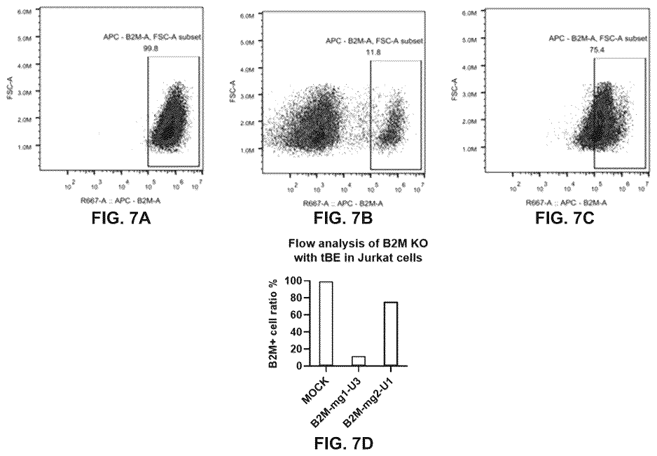

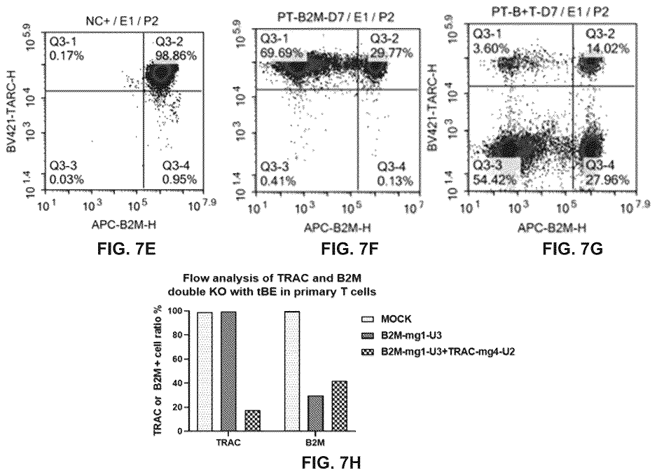

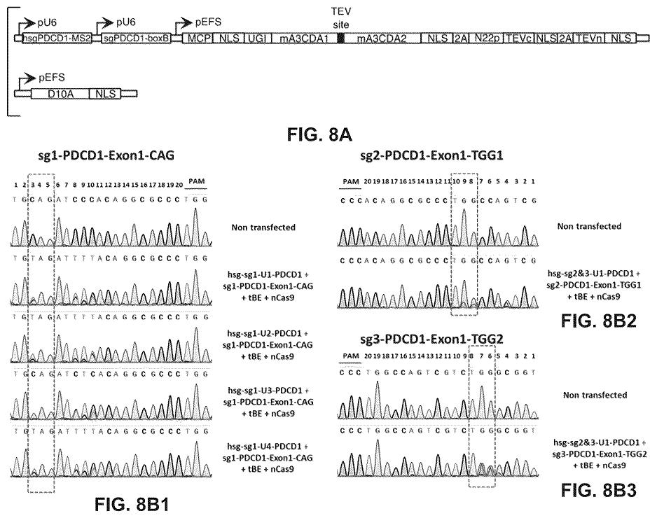

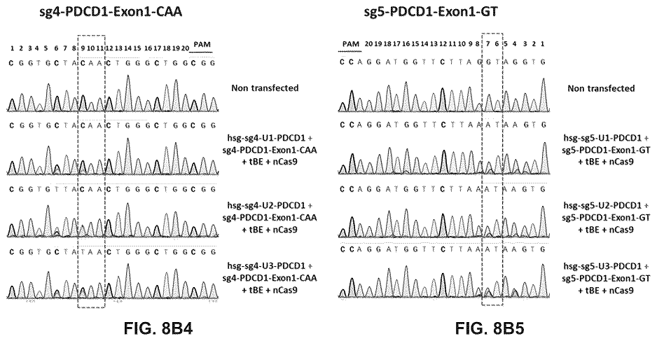

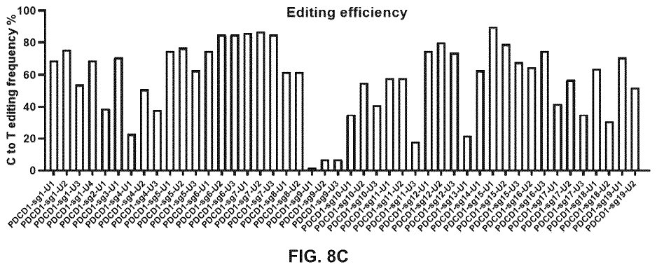

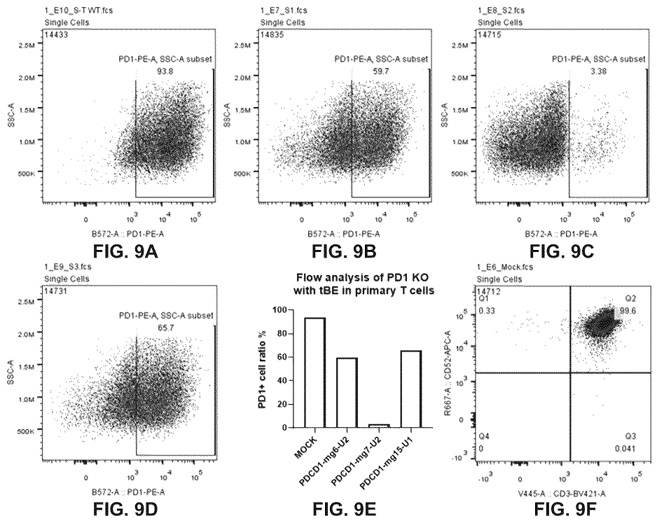

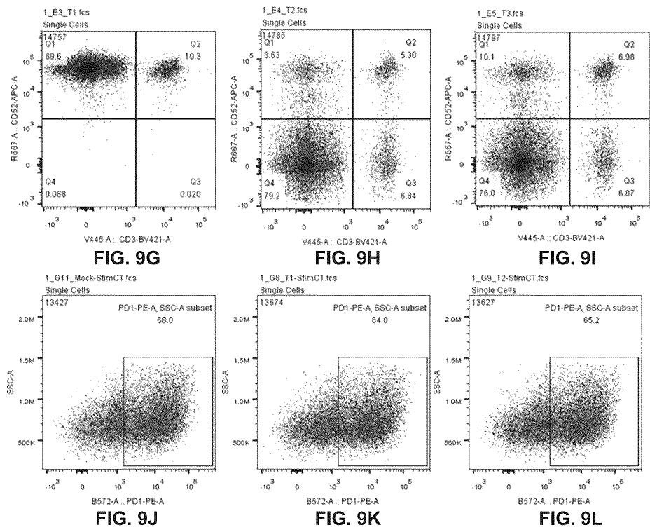

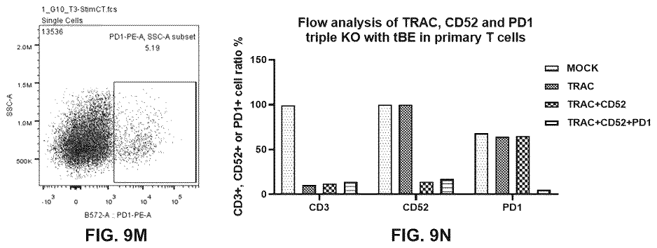

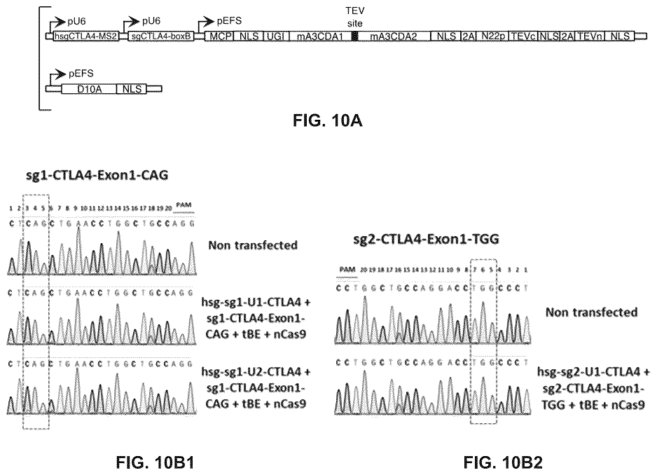

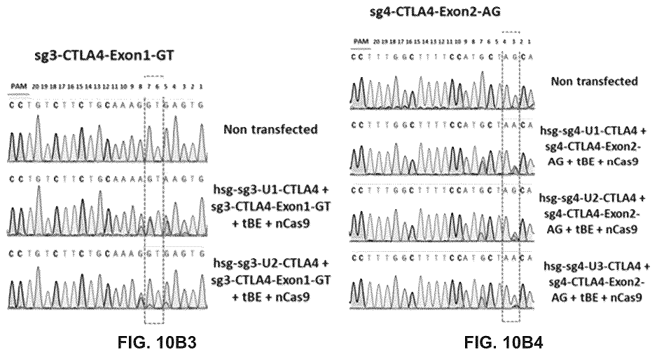

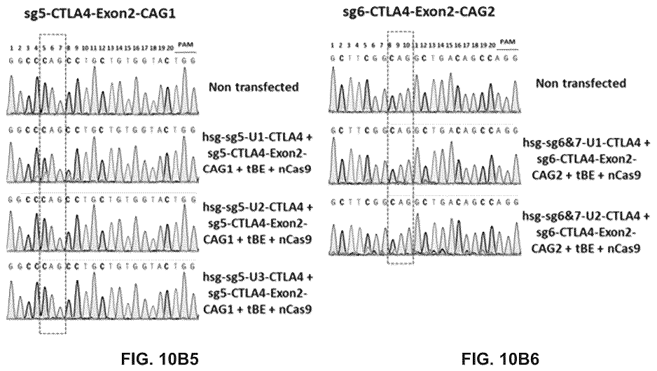

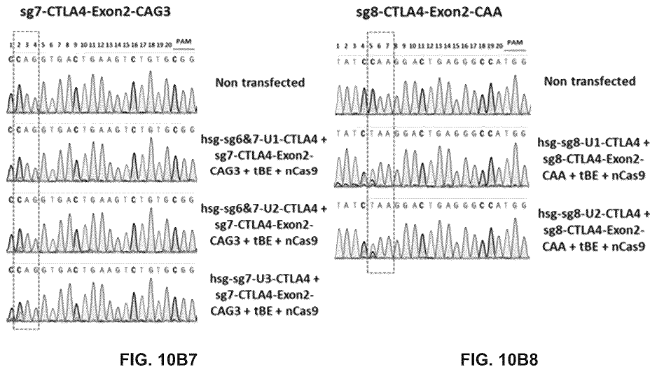

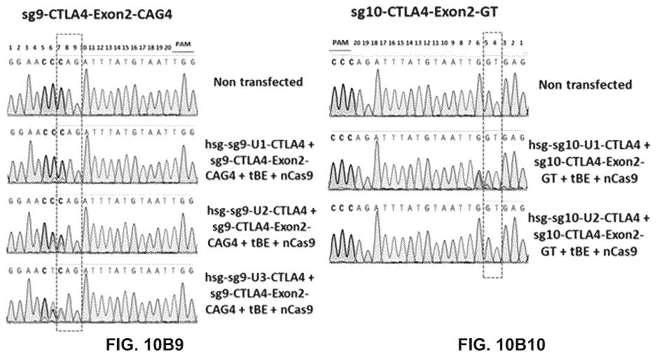

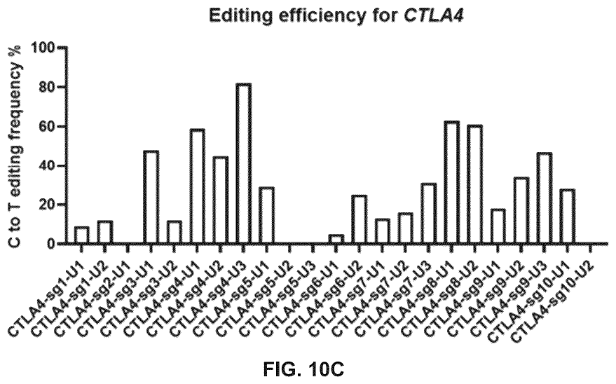

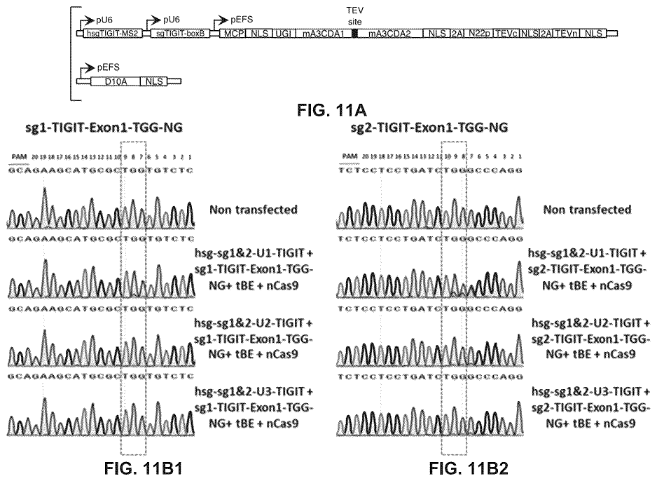

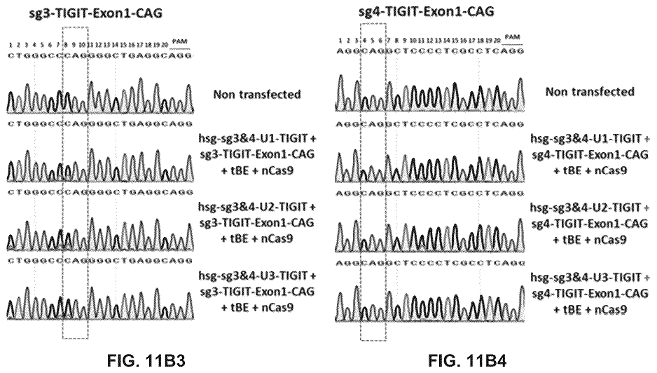

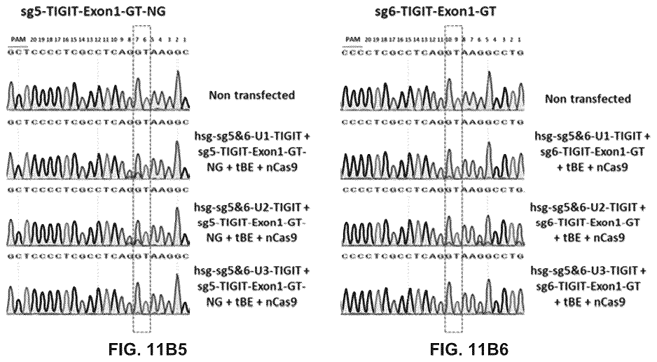

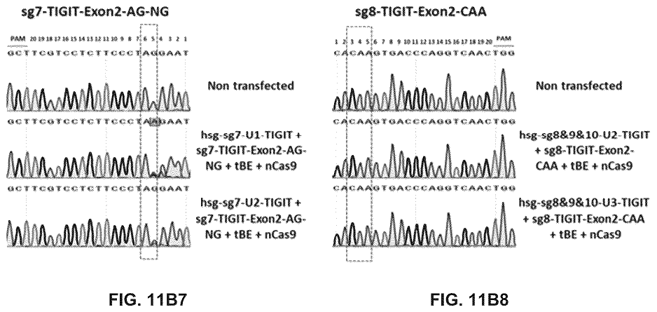

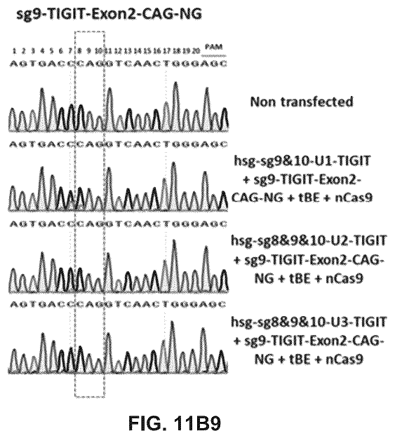

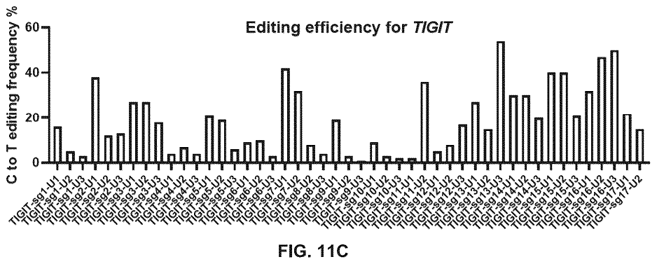

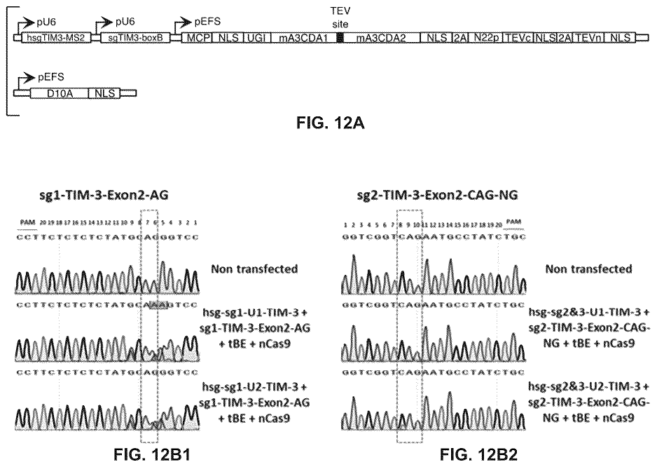

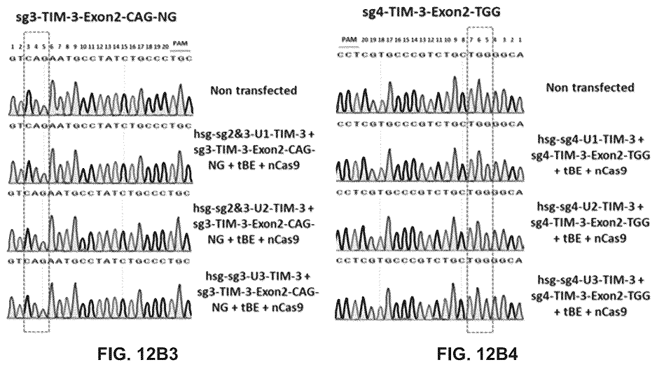

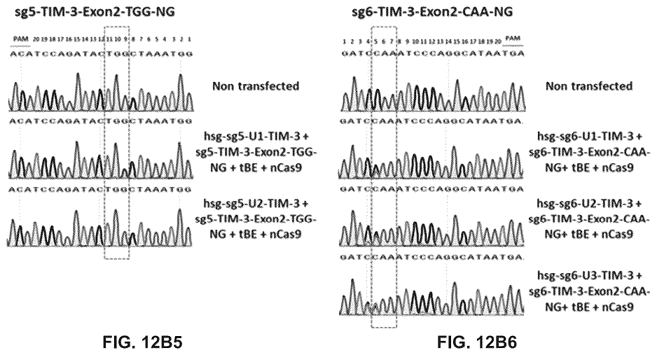

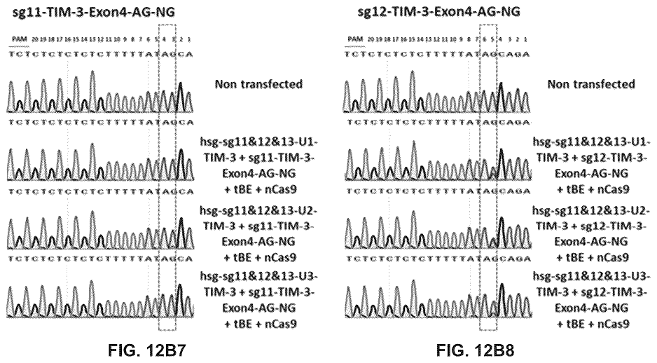

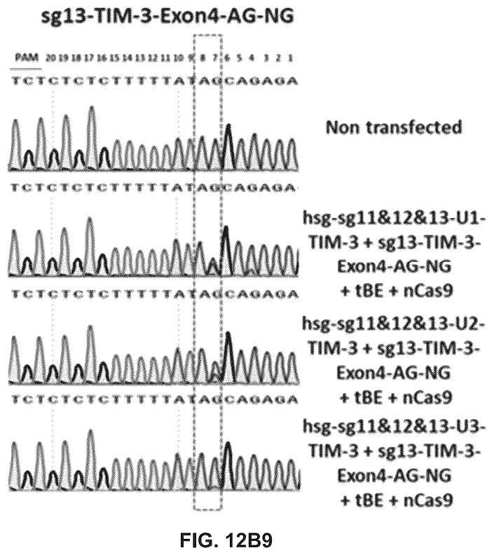

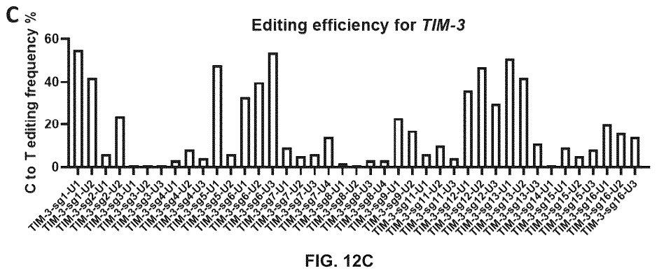

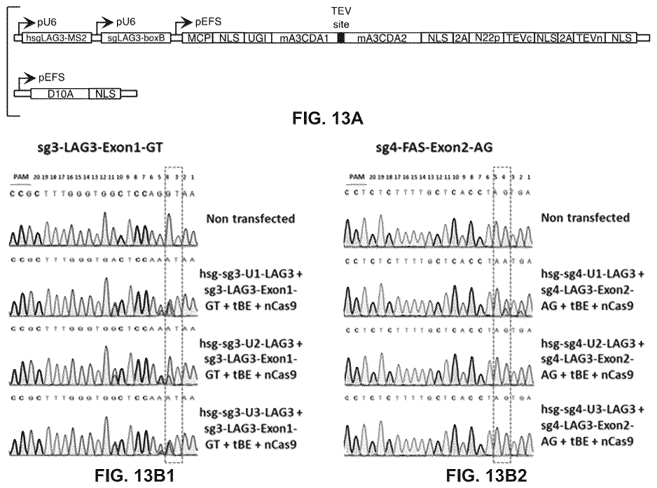

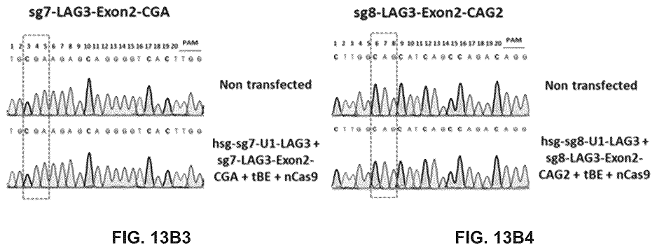

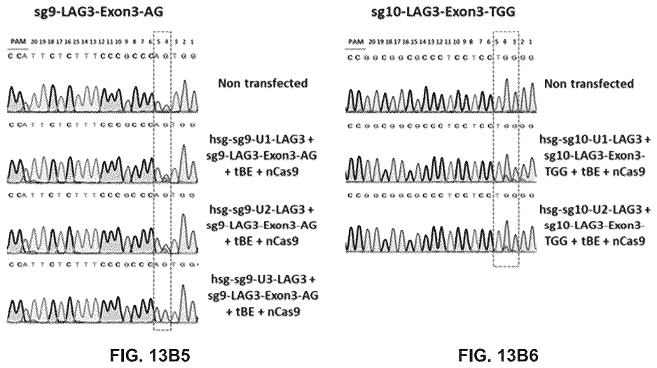

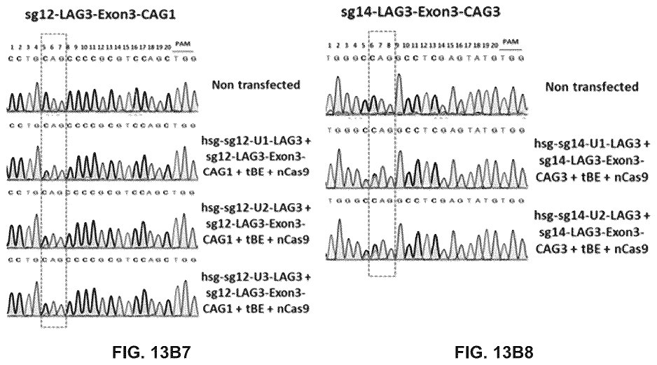

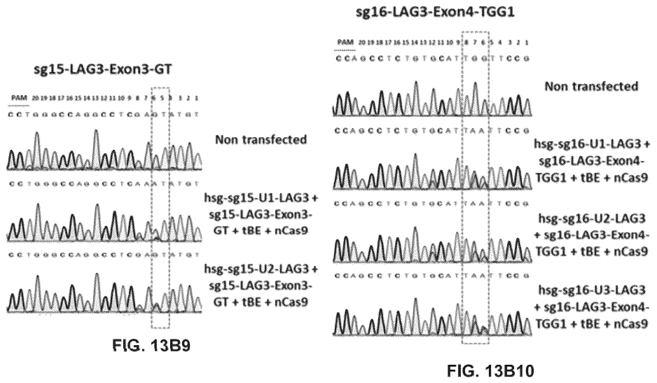

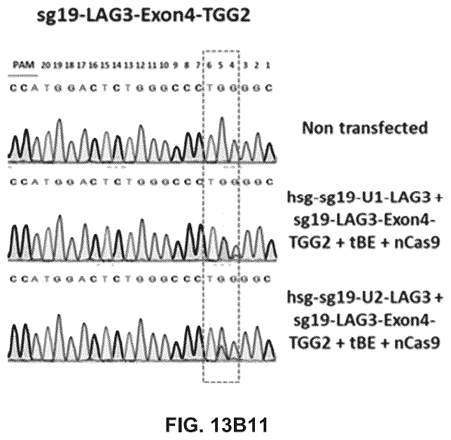

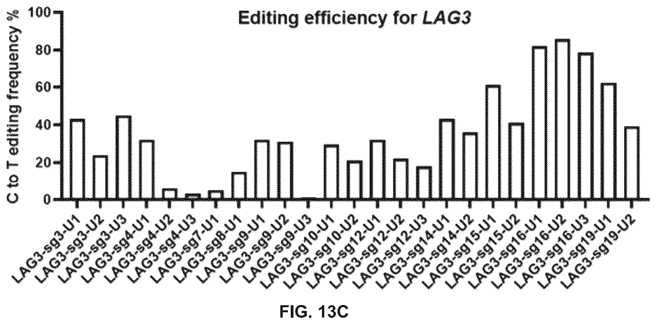

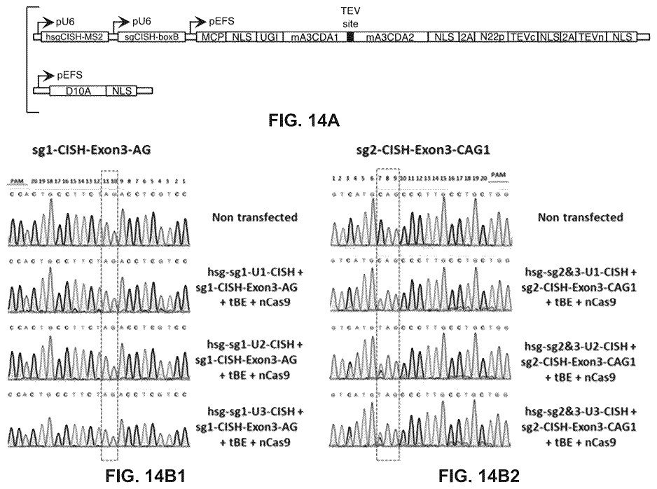

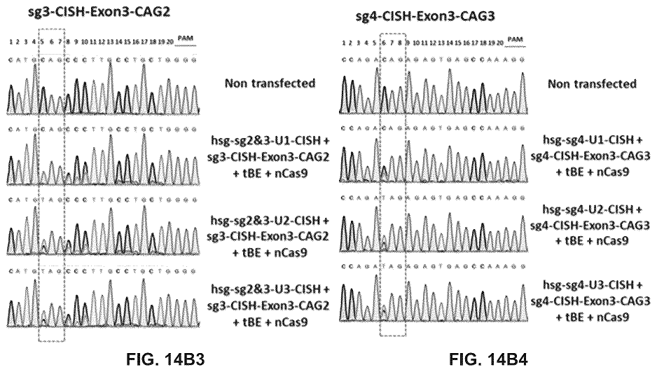

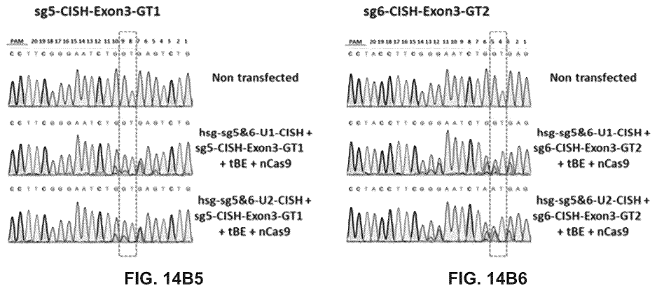

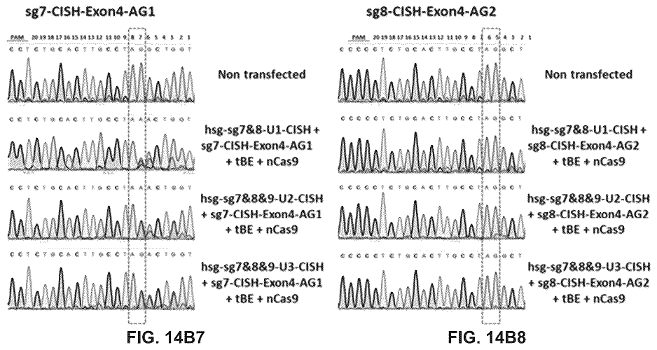

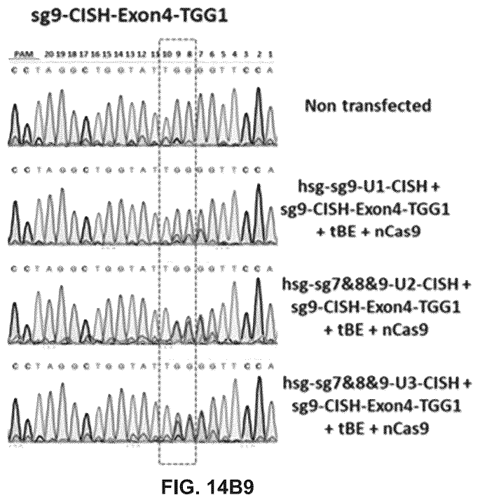

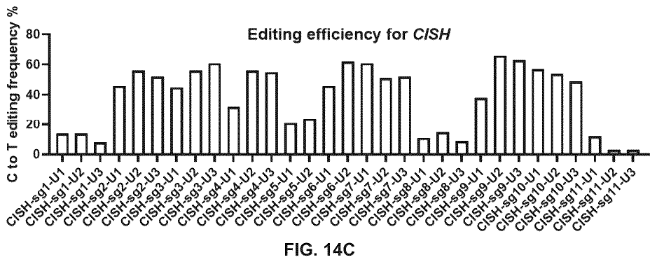

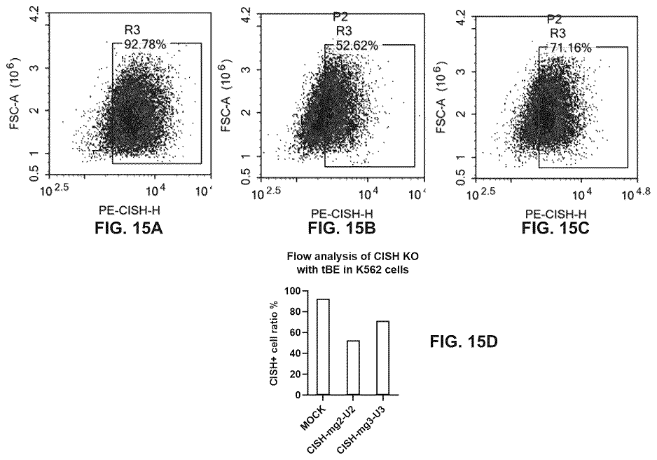

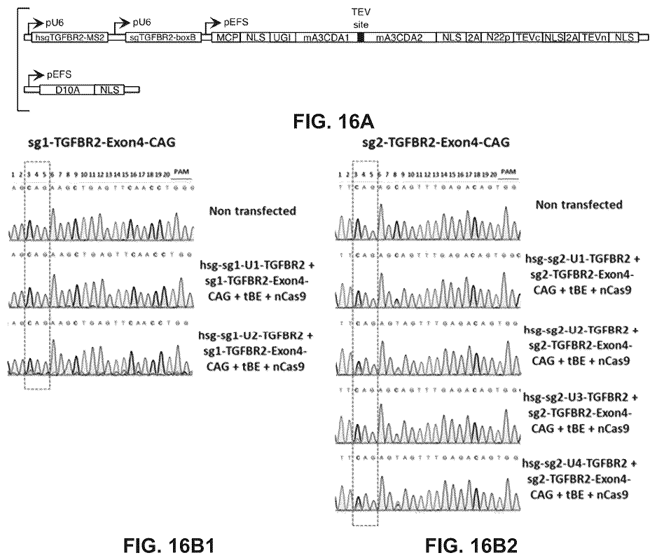

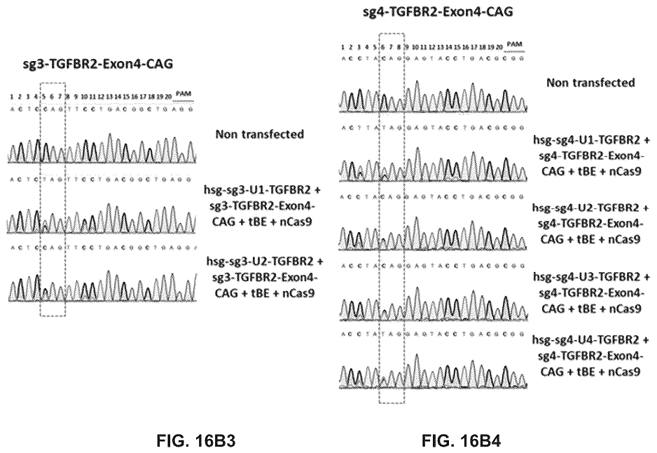

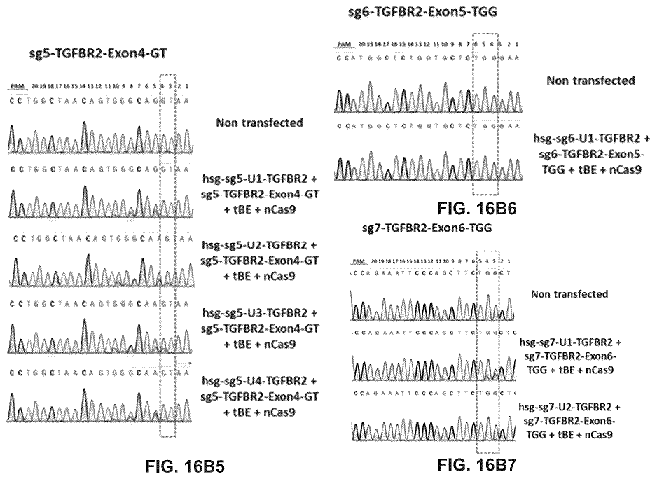

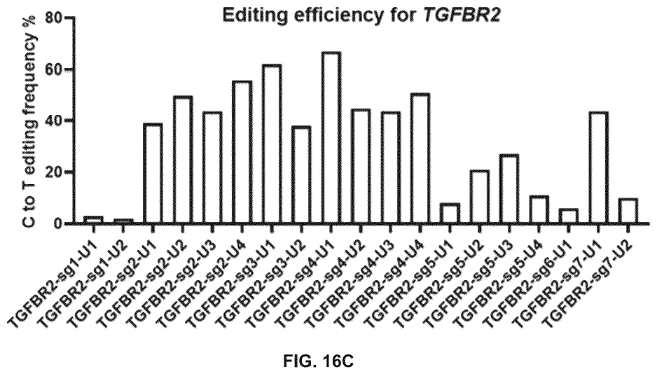

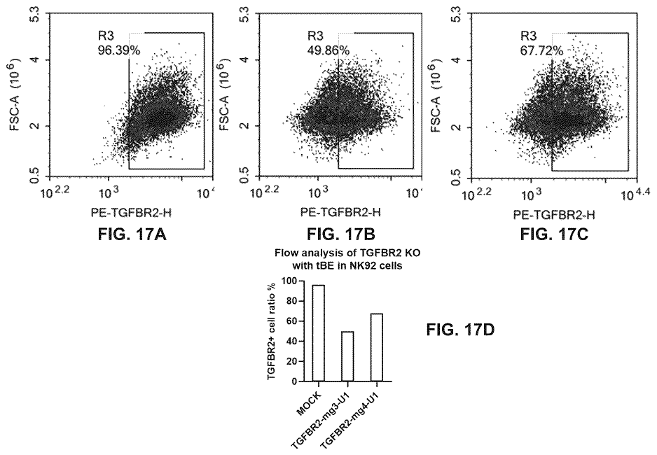

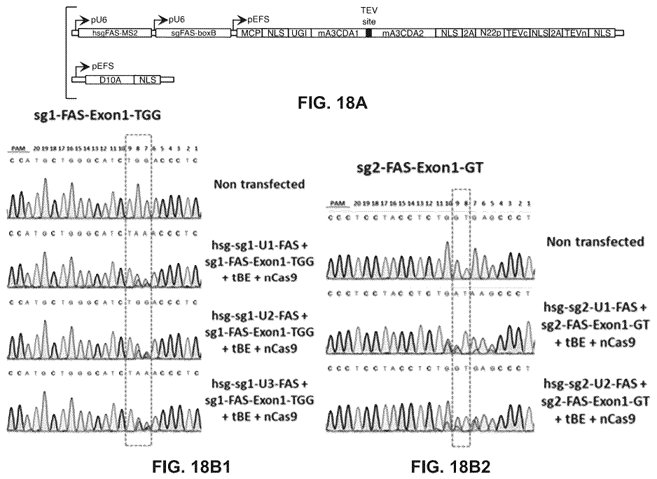

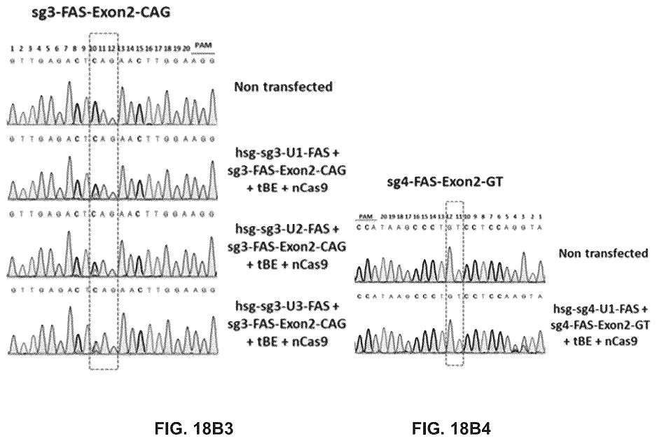

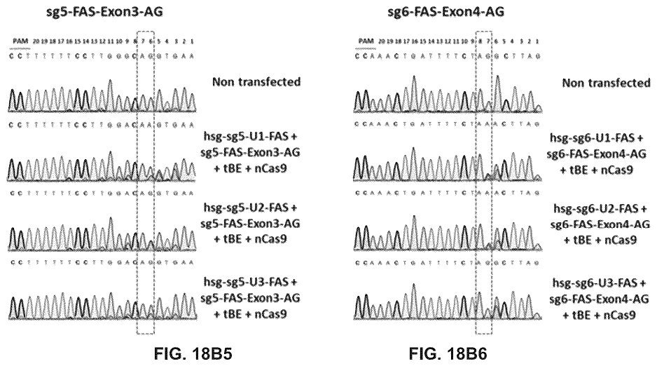

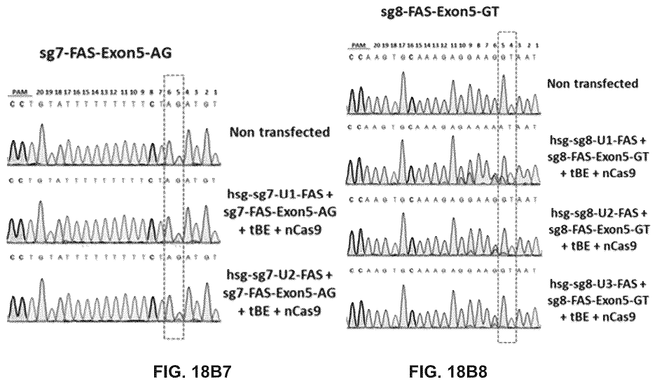

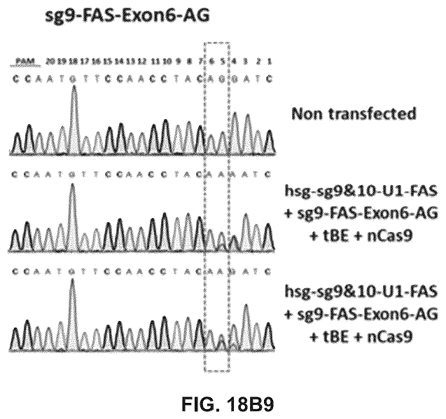

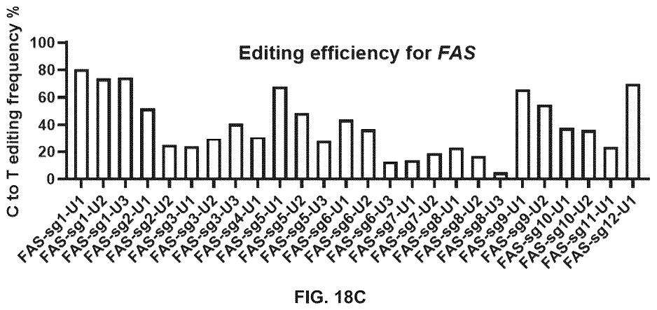

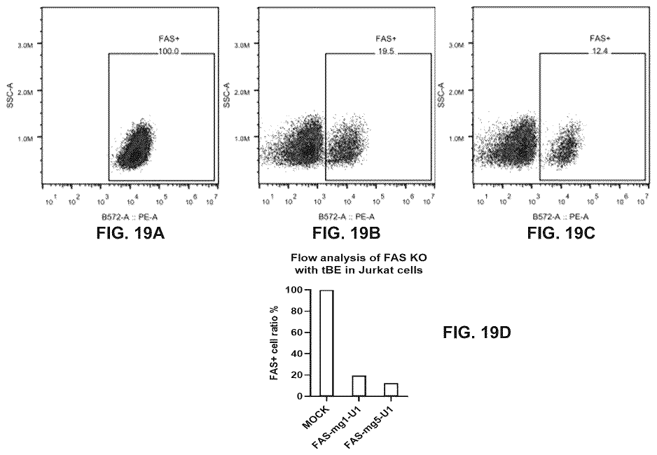

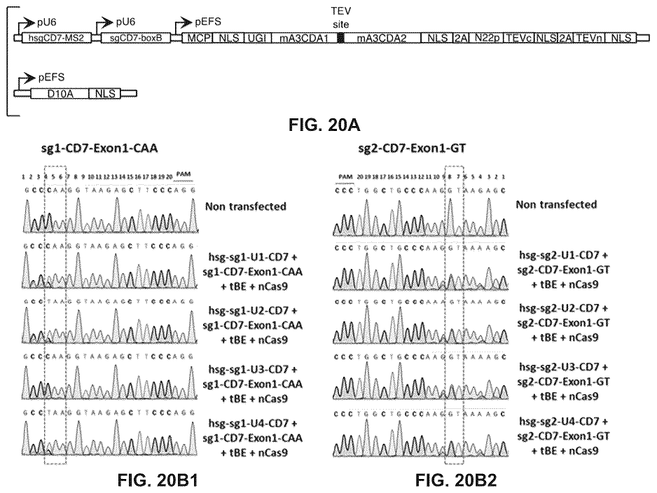

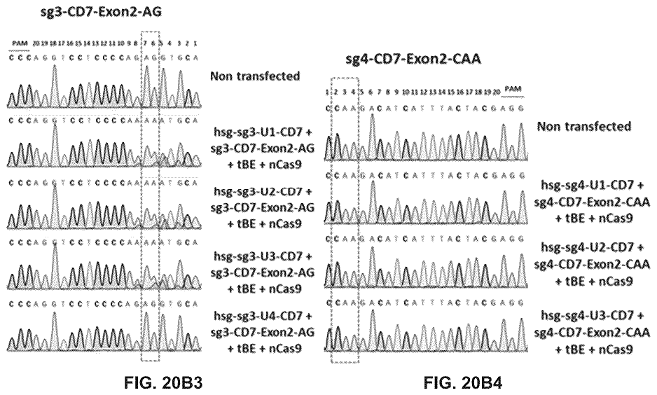

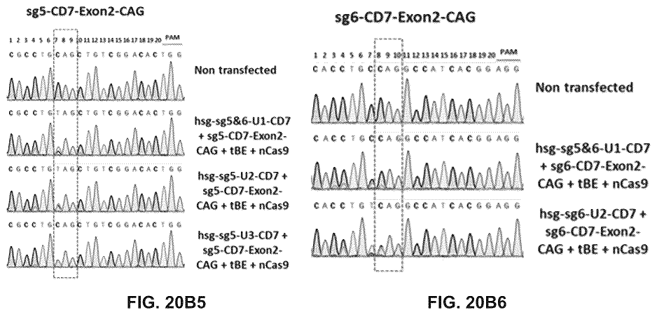

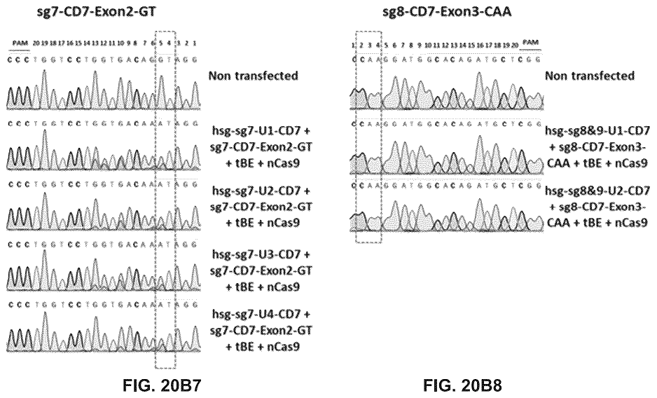

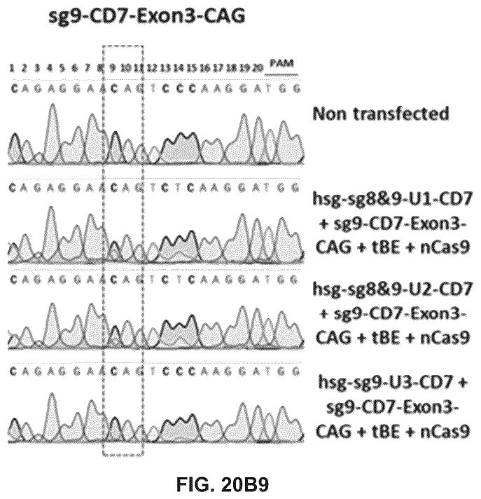

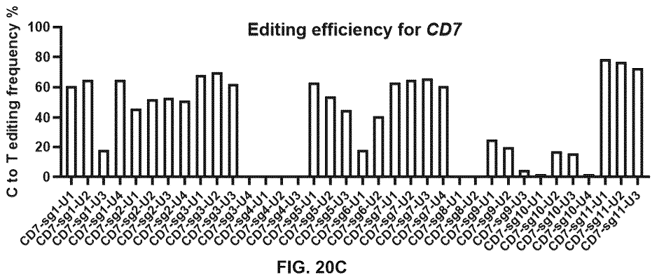

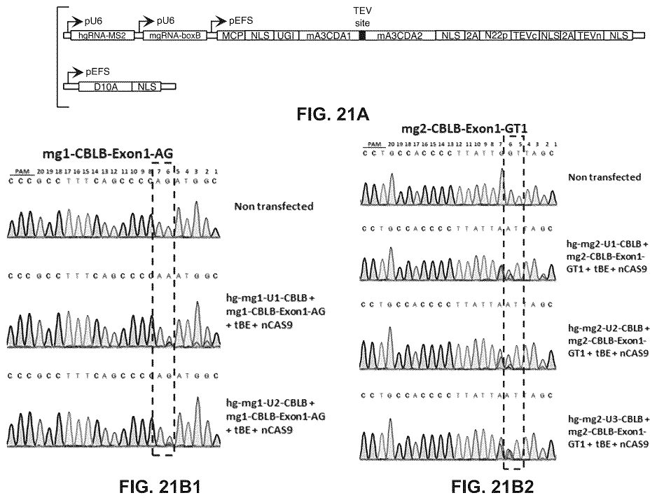

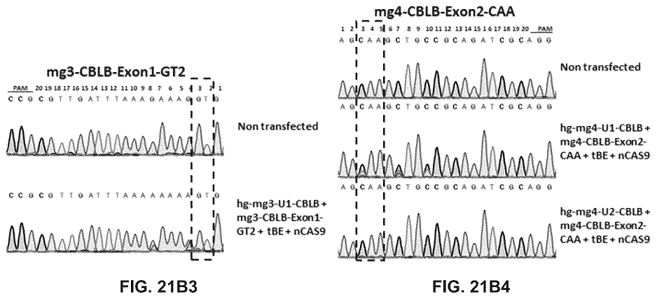

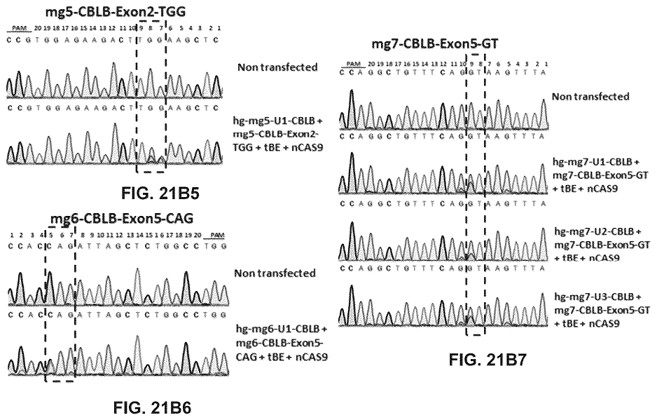

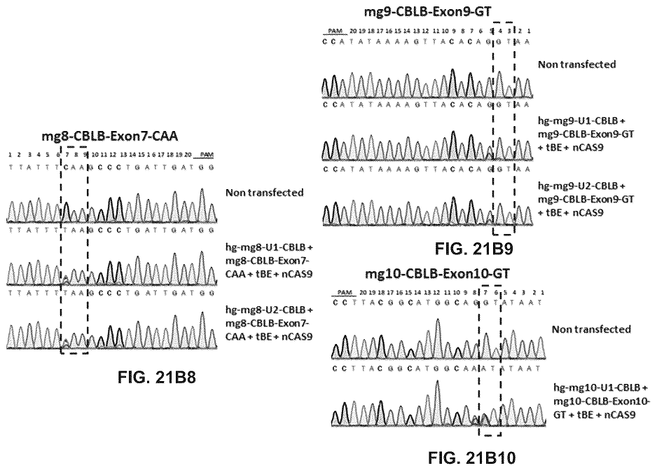

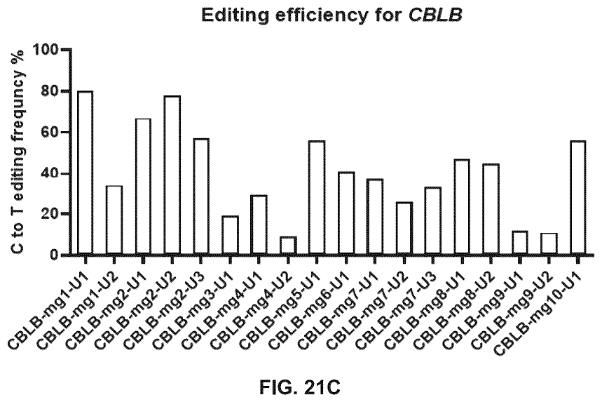

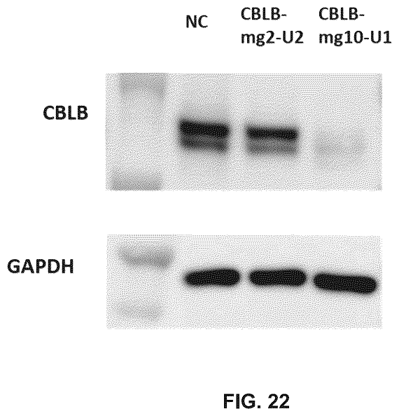

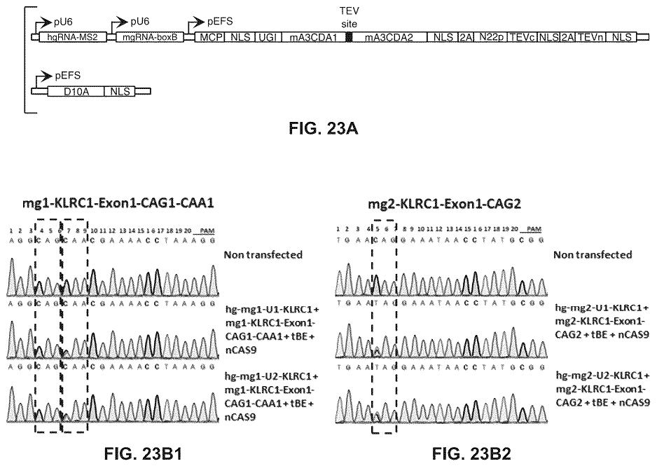

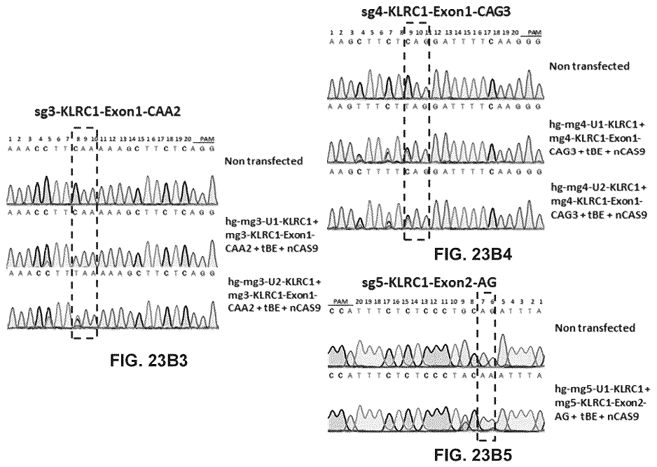

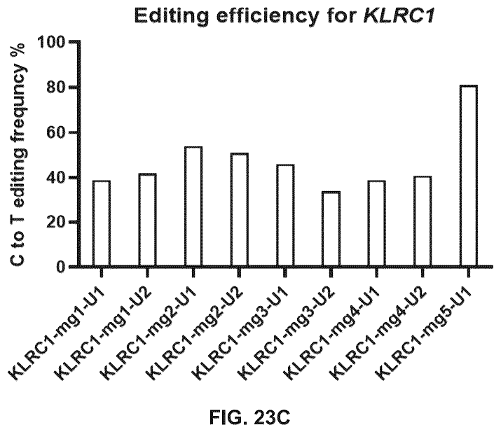

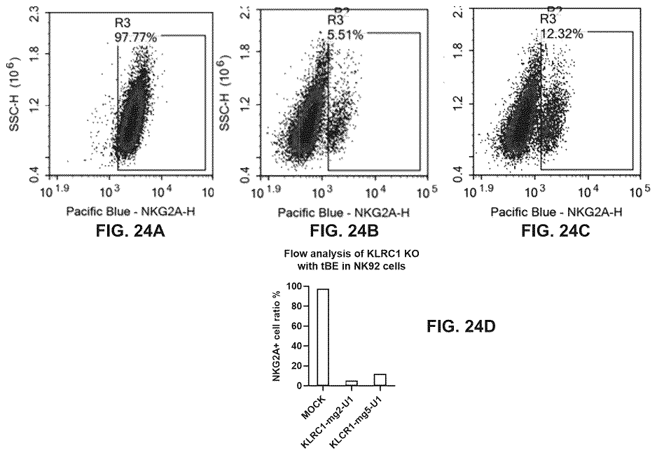

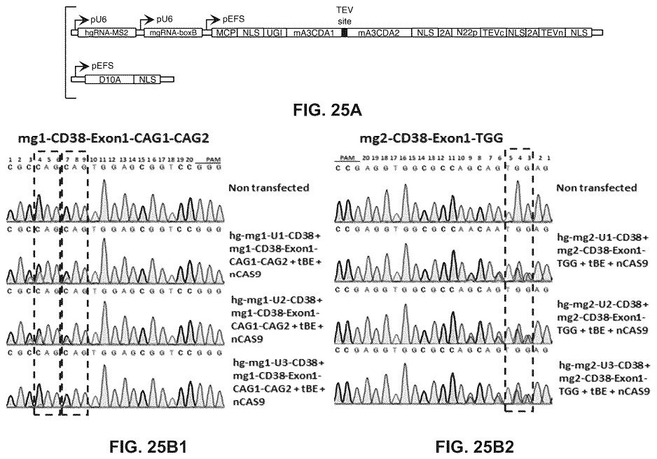

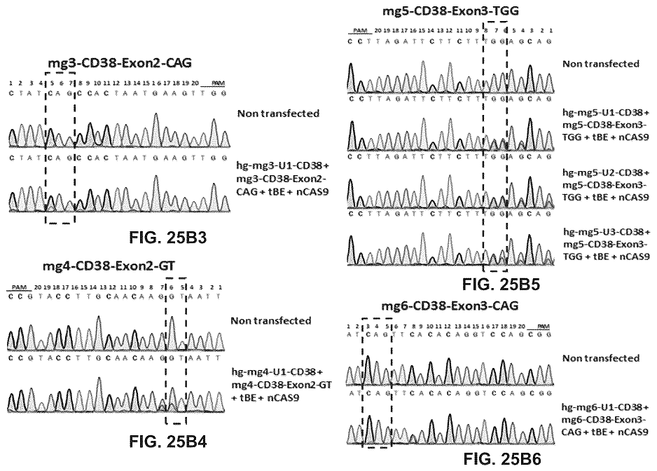

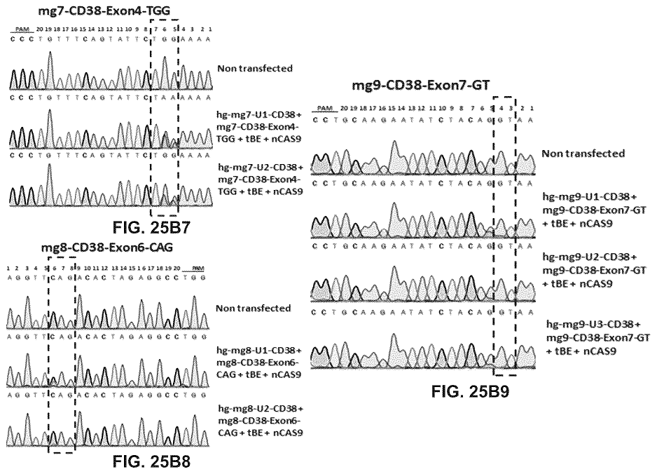

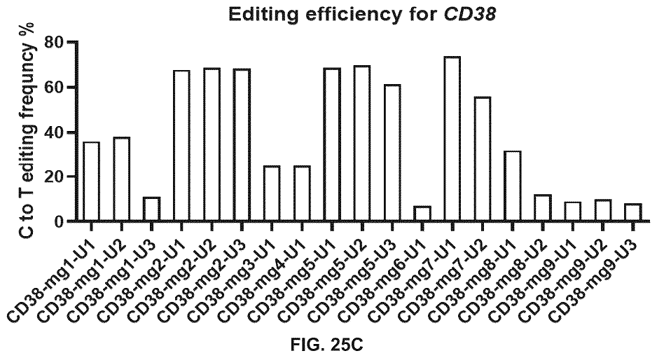

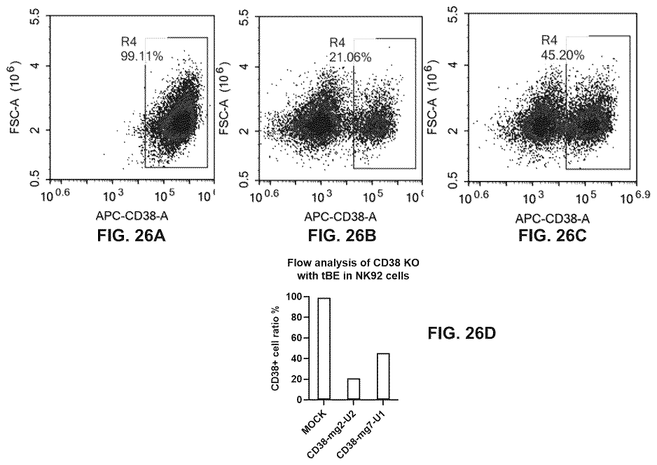

Base substitution calculation, statistics analysis, and other relevant steps for obtaining the data as illustrated in FIGS. 2-5 are essentially the same as disclosed in the “Methods” section of Wang, Lijie, et al., Eliminating base-editor-induced genome-wide and transcriptome-wide off-target mutations, Nature Cell Biology 23.5 (2021): 552-563, the content of which is incorporated herein by reference in its entirety.

Gene editing results obtained from the above experiments are illustrated in FIGS. 2-5.

### Verification on the Protein Level

The results of tBE editing were further verified by the protein level change with flow cytometry or western blot. The mgRNA/hgRNA pairs that showed relatively higher editing efficiency for the TRAC, B2M, CD52, PDCD1, CISH, TGFBR2, FAS, CBLB, KLRC1 or CD38 gene, either each gene alone or two to three genes in combination, were tested. The results confirmed that tBE induced C-to-T or G-to-A base editing could disrupt the protein expression of the target genes, whether singlex, duplex or triplex editing.

While the disclosure has been particularly shown and described with reference to specific embodiments, it should be understood by those having skill in the art that various changes in form and detail may be made therein without departing from the spirit and scope of the present disclosure as disclosed herein.

The sequences SEQ ID NOs: 828-920 are as follows:

| | | |
|---|---|---|
| Agrobacterium | MAERTHFMELALVEARSAGERDEVPIGAVLVLDGRVIARSGNRTR | SEQ |
| fabrum | ELNDVTAHAEIAVIRMACEALGQERLPGADLYVTLEPCTMCAAAI | ID |
| TadA | SFARIRRLYYGAQDPKGGAVESGVRFFSQPTCHHAPDVYSGLAES | NO: |
| | ESAEILRQFFREKRLDD | 828 |
| Arabidopsis | MFNTYTNSLQWPIRSRNQQDYCSLLPERSESYKLSKAYTSSRCYC | SEQ |
| thaliana | VSSRSSCCCCCSTPSSSSFVKPKVLINPGFVLYGVRQSTLIQWPSFQ | ID |
| TadA | RRLLVGGGRLMGCEVYSSCDGIRRKNRSFKLRCLEESDECCGGRS | NO: |
| | CSDDVEAMISFLSEELIDEERKWNLVSRVKEKKKVGNVRKVSVEG | 829 |
| | SNSYGNGRVSQRVKKPEGFGRRKEIKEDVKLNERYDCEHCGRRK | |
| | KSSELESESRRGSKLVTGEYIGKSYRGDEEREVRPRRRKSSSCSSY | |
| | YSLASSGEFESDTEDQEEDVEIYRENVRSSEKKVVDQSAKRLKSR | |
| | KEASQMHSRKKRDESSTGVDSRYQKQIFEEGENSNQAVTLNQRR | |
| | RKKFSQTENRVSESTGNYEEDMEIHEVHVNDAETSSQNQKLFNER | |
| | EDYRVHSIRNDSGNENIESSQHQLKERLETRYSSEDRVSEMRRRTK | |
| | YSSSQEEGINVLQNFPEVTNNQQPLVEERISKQAGTRRTTEHISESS | |
| | EIHDIDIRNTYVSQREDQIRNQEVHAGLVSGLQSERKQQDYHIEHN | |
| | PLQTTQSDRTSVSVSHTSDAVRYTEIQRKSEKRLIGQGSTTAVQSD | |
| | SKVEKNGAQKEDSRLDHANSKKDGQTTLGLQSYQSKLSEEASSS | |
| | QSSLMASRTKLQLVDLVSEEMQGSETTLIPPSSQLVSRRSGQSYRT | |
| | GGVSIQEISHGTSESGYTTAFEHPRAGASVNSQSAGELMGFTSHED | |
| | AMGSAHRLEQASEKYVGEFVKKAKHGVINPETEEQRAESNQLKR | |
| | RDSRRSSGGSGAKGPSDEMWVTDSAQGTPHPGATEGNAAVGNAI | |
| | FKRNGRSLWNVIADIARLRWGSRAGSPDSSAKPAGRSSPNESVSS | |
| | ATWFSGREHDGSSDDNTKGDKVLPQEAPSLHQVEVGQTSPRSQSE | |
| | YPGTTKLKQRSERHEGVVSSPSSTILEGGSVSNRMSSTSGNQIVGV | |
| | DEEEGGNFEFRLPETALTEVPMKLPSRNLIRSPPIKESSESSLTEASS | |
| | DQNFTVGEGRRYPRMDAGQNPLLFPGRNLRSPAVMEPPVPRPRM | |
| | VSGSSSLREQVEQQQPLSAKSQEETGSVSADSALIQRKLQRNKQV | |
| | VRDSFEEWEEAYKVEAERRTVDEIFMREALVEAKKAADTWEVPV | |
| | GAVLVHDGKIIARGYNLVEELRDSTAHAEMICIREGSKALRSWRL | |
| | ADTTLYVTLEPCPMCAGAILQARVNTLVWGAPNKLLGADGSWIR | |
| | LFPGGEGNGSEASEKPPPPVHPFHPKMTIRRGVLESECAQTMQQFF | |
| | QLRRKKKDKNSDPPTPTDHHHHHLPKLLNKMHQVLPFFCL | |
| Aquifexacolicus | MGKEYFLKVALREAKRAFEKGEVPVGAIIVKEGEIISKAHNSVEEL | SEQ |
| TadA | KDPTAHAEMLAIKEACRRLNTKYLEGCELYVTLEPCIMCSYALVL | ID |
| | SRIEKVIFSALDKKHGGVVSVFNILDEPTLNHRVKWEYYPLEEASE | NO: |
| | LLSEFFKKLRNNII | 830 |
| Streptococcus | MPYSLEEQTYFMQEALKEAEKSLQKAEIPIGCVIVKDGEIIGRGHN | SEQ |
| pycgenes | AREESNQAIMHAEMMAINEANAHEGNWRLLDTTLFVTIEPCVMC | ID |
| serotype | SGAIGLARIPHVIYGASNQKFGGADSLYQILTDERLNHRVQVERGL | NO: |
| M3 TadA | LAADCANIMQTFFRQGRERKKIAKHLIKEQSDPFD | 831 |
| Bacillus | MTQDELYMKEAIKEAKKAEEKGEVPIGAVLVINGEIIARAHNLRE | SEQ |
| subtilis | TEQRSIAHAEMLVIDEACKALGTWRLEGATLYVTLEPCPMCAGA | ID |
| TadA | VVLSRVEKVVFGAFDPKGGCSGTLMNLLQEERFNHQAEVVSGVL | NO: |
| | EEECGGMLSAFFRELRKKKKAARKNLSE | 832 |
| Haemophilus | MDAAKVRSEFDEKMMRYALELADKAEALGEIPVGAVLVDDARN | SEQ |
| influenzae | IIGEGWNLSIVQSDPTAHAEIIALRNGAKNIQNYRLLNSTLYVTLEP | ID |
| TadA | CTMCAGAILHSRIKRLVFGASDYKTGAIGSRFHFFDDYKMNHTLE | NO: |
| | VTSGVLAEECSQKLSTFFQKRREEKKIEKALLKSLSDK | 833 |
| Buchnera | MKYEKDKNWMKIALKYAYYAKEKGEIPIGAILVFKERIIGIGWNS | SEQ |
| aphidicola | SISKNDPTAHAEIIALRGAGKKIKNYRLLNTTLYVTLQPCIMCCGAI | ID |
| TadA | IQSRIKRLVFGANCNSSDHRFSLKNLFCDPQKDYKLDIKKNVMQR | NO: |
| | ECSDILINFFQKKRKNKIHICKKI | 834 |
| Escherichia | MSEVEFSHEYWMRHALTLAKRAWDEREVPVGAVLVHNNRVIGE | SEQ |
| coli | GWNRPIGRHDPTAHAEIMALRQGGLVMQNYRLIDATLYVTLEPC | ID |
| TadA | VMCAGAMIHSRIGRVVFGARDAKTGAAGSLMDVLHHPGMNHRV | NO: |
| | EITEGILADECAALLSDFFRMRRQEIKAQKKAQSSTD | 835 |
| Streptococcus | MPYSLEEQTYFMQEALKEAEKSLQKAEIPIGCVIVKDGEIIGRGHN | SEQ |
| pycgenes | AREESNQAIMHAEMMAINEANAHEGNWRLLDTTLFVTIEPCVMC | ID |
| serotype | SGAIGLARIPHVIYGASNQKFGGVDSLYQILTDERLNHRVQVERGL | NO: |
| M1 TadA | LAADCANIMQTFFRQGRERKKIAKHLIKEQSDPFD | 836 |
| Rickettsia | MREALKQAEIAFSKNEVPVGAVIVDRENQKIISKSYNNTEEKNNA | SEQ |
| bellii | LYHAEIIAINEACRIISSKNLSDYDIYVTLEPCAMCAAAIAHSRLKR | ID |
| TadA | LFYGASDSKHGAVESNLRYFNSKACFHRPEIYSGIFAEDSALLMK | NO: |
| | GFFKKIRD | 837 |
| Rickettsia | MEQALKQAGIAFDKNEVPVGAVIVDRLNQKIIVSSHNNTEEKNNA | SEQ |
| felis TadA | LYHAEIIAINEACNLISSKNLNDYDIYVTLEPCAMCAAAIAHSRLK | ID |
| | RLFYGASDSKHGAVESNLRYFNSSVCFYRPEIYSGILAEDSRLLMK | NO: |
| | EFFKRIR | 838 |
| Salmonella | MSDVELDHEYWMRHALTLAKRAWDEREVPVGAVLVHNHRVIGE | SEQ |
| typhimurium | GWNRPIGRHDPTAHAEIMALRQGGLVLQNYRLLDTTLYVTLEPC | ID |
| TadA | VMCAGAMVHSRIGRVVFGARDAKTGAAGSLIDVLHHPGMNHRV | NO: |
| | EIIEGVLRDECATLLSDFFRMRRQEIKALKKADRAEGAGPAV | 839 |
| Escherichia | MSEVEFSHEYWMRHAMTLAKRAWDEREVPVGAVLVHNNRVIGE | SEQ |
| coli 06 | GWNRPIGRHDPTAHAEIMALRQGGLVMQNYRLIDATLYVTLEPC | ID |
| TadA | VMCAGAMIHSRIGRVVFGARDAKTGAAGSLMDVLHHPGMNHRV | NO |
| | EITEGILADECAALLSDFFRMRRQEIKAQKKAQSSTD | 840 |
| Buchnera | MKSNRDSYWMKIALKYAYYAEENGEVPIGAILVFQEKIIGTGWNS | SEQ |
| aphidicola | VISQNDSTAHAEIIALREAGRNIKNYRLVNTTLYVTLQPCMMCCG | ID |
| subsp. | AIINSRIKRLVFGASYKDLKKNPFLKKIFINLEKNKLKIKKHIMRNE | NO: |
| Baizongia | CAKILSNFFKNKRF | 841 |
| pistaciae | | |
| (strain Bp) | | |
| TadA | | |
| Streptococcus | MPYSLEEQTYFMQEALKEAEKSLQKAEIPIGCVIVKDGEIIGRGHN | SEQ |
| pycgenes | AREESNQAIMHAEMMAINEANAHEGNWRLLDTTLFVTIEPCVMC | ID |
| serotype | SGAIGLARIPHVIYGASNQKFGGADSLYQILTDERLNHRVQVERGL | NO: |
| M18 | LAADCANIMQTFFRQGRERKKNS | 842 |
| TadA | | |
| Rickettsia | MEQALKQARLAFDKNEVPVGVVIVCRLNQKIIVSSHNNIEEKKNP | SEQ |
| prowazekii | LCHAEIIAINTACNLISSKNLNDYDIYVTLEPCAMCASAISHSRLKR | ID |
| TadA | LFYGASDSKHGAVESNLRYFNSNSCFYRPEIYSGILSEHSRFLMQE | NO: |
| | FFQRIRSAID | 843 |
| Rickettsia | MEQALKQARLAFDKNEVPVGVVIVYRLNQKIIVSSHNNIEEKNNA | SEQ |
| typhi | LCHAEIIAINEACNLISSKNLNDYDIYVTLEPCAMCASAISHSRLKR | ID |
| TadA | LFYGASDSKQGAVESNLRYFNSSACFHRPEIYSGILSEHSRFLMKE | NO: |
| | FFQKMRSTID | 844 |
| Buchnera | MHDSDKYFMKCAIFLAKISEMIGEVPVGAVLVFNNTIIGKGLNSSI | SEQ |
| aphidicola | LNHDPTAHAEIKALRNGAKFLKNYRLLHTTLYVTLEPCIMCYGAII | ID |
| subsp. | HSRISRLVFGAKYKNLQKYICCKNHFFINKNFRKISITQEVLESECS | NO: |
| Schizaphis | NLLSSFFKRKRKIATKYENNNII | 845 |
| graminum | | |
| (strain Sg) | | |
| TadA | | |
| Rickettsia | MEQALKQAKIAFDKNEVPVGAVVVDRLHQKIIASTHNNTEEKNN | SEQ |
| conorii | ALYHAEIIAINEACNLISSKNLNDYDIYVTLEPCAMCAAAIAHSRL | ID |
| TadA | KRLFYGASDSKHGVVESNLRYFNSSACFHRPEIYSGILAEDSGLLM | NO: |
| | KEFFKRIRTVISSHRMT | 846 |
| Staphylococcus | MTNDIYFMTLAIEEAKKAAQLGEVPIGAIITKDDEVIARAHNLRET | SEQ |
| aureus | LQQPTAHAEHIAIERAAKVLGSWRLEGCTLYVTLEPCVMCAGTIV | ID |
| TadA | MSRIPRVVYGADDPKGGCSGSLMNLLQQSNFNHRAIVDKGVLKE | NO: |
| | ACSTLLTTFFKNLRANKKSTN | 847 |
| Arabidopsis | MEEDWGKTVSEKVISAYMSLPKKGKPQGREVTVLSAFLVSSPSQD | SEQ |
| thaliana | PKVIALGTGTKCVSGSLLSPRGDIVNDSHAEVVARRALIRFFYSEIQ | ID |
| ADAT1 | RMQLTSGKSNEAKRQRIDSETSSILESADSSCPGEVKYKLKSGCLL | NO: |
| | HLYISQLPCGYASTSSPLYALKKIPSTQVDDSLLVQASDICSSRHSD | 848 |
| | VPEIGSNSNKGNGSQVADMVQRKPGRGETTLSVSCSDKIARWNV | |
| | LGVQGALLYQVLQPVYISTITVGQSLHSPDNFSLADHLRRSLYERI | |
| | LPLSDELLTSFRLNKPLFFVAPVPPSEFQHSETAQATLTCGYSLCW | |
| | NYSGLHEVILGTTGRKQGTSAKGALYPSTQSSICKQRLLELFLKET | |
| | HGHKRESSKSKKSYRELKNKATEYYLMSKIFKGKYPFNNWLRKP | |
| | LNCEDFLIN | |
| Schizosac | MEEFTLDRNSNVGNLIALAVLNKFDELARHGKPIIRANGVREWTT | SEQ |
| charomyces | LAGVVIQKKMENEFICVCLATGVKCTPAGIIKNEQLGSVLHDCHA | ID |
| pombe | EILALRCFNRLLLEHCILIKESKKDTWLLEVADNGKFTLNSNLLIHL | NO: |
| ADAT1 | YVSECPCGDASMELLASRLENNKPWNLTVDSEKLMRGRADFGLL | 849 |
| | GIVRTKPGRPDAPVSWSKSCTDKLAAKQYLSILNSQTSLICEPIYLS | |
| | CVVLYKKVIVKSAIDRAFGPFGRCAPLAEFGEKDNPYYFHPFTVLE | |
| | TDENFLYSRPLNQAEKTATSTNVLIWIGDKMQCTQVIHNGIKAGT | |
| | KAKDVEKSQTLICRKSMMNLLHQLSQSLTNEKNYYEWKKLNIKR | |
| | CQQKQILRNILKNWIPNGGNEFQWI | |
| Saccharomyces | MVSCQGTRPCIVNLLTMPSEDKLGEEISTRVINEYSKLKSACRPIIR | SEQ |
| cerevisiae | PSGIREWTILAGVAAINRDGGANKIEILSIATGVKALPDSELQRSEG | ID |
| ADAT1 | KILHDCHAEILALRGANTVLLNRIQNYNPSSGDKFIQHNDEIPARF | NO: |
| | NLKENWELALYISRLPCGDASMSFLNDNCKNDDFIKIEDSDEFQY | 850 |
| | VDRSVKTILRGRLNFNRRNVVRTKPGRYDSNITLSKSCSDKLLMK | |
| | QRSSVLNCLNYELFEKPVFLKYIVIPNLEDETKHHLEQSFHTRLPN | |
| | LDNEIKFLNCLKPFYDDKLDEEDVPGLMCSVKLFMDDFSTEEAIL | |
| | NGVRNGFYTKSSKPLRKHCQSQVSRFAQWELFKKIRPEYEGISYL | |
| | EFKSRQKKRSQLIIAIKNILSPDGWIPTRTDDVK | |
| Macaca | MWTADEIALLCYEHYGIRLPKKGKPEPNHEWTLLAAVVKIQSPAD | SEQ |
| fascicularis | QDCDTPDKPAQVTKEVVSMGTGTKCIGQSKMRKSGDILNDSHAE | ID |
| ADAT1 | VIARRNFQRYLLHQLQLAATLKEDSIFVPGTQKGLWKLRRDLFFV | NO: |
| | FFSSHTPCGDASIIPMLEFEDQPCCPVIRDWASSSSVEASSNLEAPG | 851 |
| | NERKCEDLDSPVTKKMRLEPMTAAREVTNGATHHQSFGKQESGP | |
| | ISPGINSCNLTVEGLAAVTRIAPGSAKVIDVYRTGAKCVPGEAGDS | |
| | RKPGAAFHQVGLLRVKPGRGDRTRSMSCSDKMARWNVLGCQGA | |
| | LLMHFLEEPIYLSAVVIGKCPYSQEAMQRALTGRRQNVSALPKGF | |
| | GVQELKILQSDLLFEQSRCAVQAKRADSPGRLVPCGAAISWSAVP | |
| | EQPLDVTANGFPQGTTKKTIGSLQARSQISKVELLRSFQKLLSRIAR | |
| | DKWPDSLRVQKLDTYQDYKEAASSYQEAWSTLRKQAFGSWIRNP | |
| | PDYHQFK | |
| Gallus | MWTADEIAELCYEHYRSRLPKQGKPDPSREWTSLAAVVKVESAA | SEQ |
| gallus | NEAGSAVLGTLQVAKEVVALGTGTKCIGLNKMRKTGDVLNDSH | ID |
| ADAT1 | AEVVAKRSFQRYLLHQMRLATSYQQCSIFIPGTETGKWKLKPNIIF | NO: |
| | IFFCSHTPCGDASIIPIRETENHLSKSVDGHDIAGQSVLCSSSNCDHR | 852 |
| | GPEDKRKSEKMASSHMIKRMKNADGGFFSTITEDMAVQQVFAKP | |
| | EGNVNPECCESSEEMQAANKETNAGKLKAVGVYRTGAKFVPGEL | |
| | SDTLIPGIEYHCVGLLRVKPGRGDRTCSMSCSDKLARWNVLGCQG | |
| | ALLMHFLQYPVYLSAVIVGKCPYSQEAMQRAVIERCRHISLLPDG | |
| | FLTQEVQLLQSDLQFEHSRQAIQEGQTSSKRKLVPCSAAISWSAVP | |
| | EGPLDVTSDGFRQGTTKKGIGSPQSRSKICKVELFHEFQKLVTSISK | |
| | ENLPDTLRMKTLETYWDYKEAALNYQEAWKALRSQALLGWIKN | |
| | AQEYLLFM | |
| Human | MWTADEIAQLCYEHYGIRLPKKGKPEPNHEWTLLAAVVKIQSPA | SEQ |
| ADAT1 | DKACDTPDKPVQVTKEVVSMGTGTKCIGQSKMRKNGDILNDSHA | ID |
| | EVIARRSFQRYLLHQLQLAATLKEDSIFVPGTQKGVWKLRRDLIFV | NO: |
| | FFSSHTPCGDASIIPMLEFEDQPCCPVFRNWAHNSSVEASSNLEAP | 853 |
| | GNERKCEDPDSPVTKKMRLEPGTAAREVINGAAHHQSFGKQKSG | |
| | PISPGIHSCDLTVEGLATVTRIAPGSAKVIDVYRTGAKCVPGEAGD | |
| | SGKPGAAFHQVGLLRVKPGRGDRTRSMSCSDKMARWNVLGCQG | |
| | ALLMHLLEEPIYLSAVVIGKCPYSQEAMQRALIGRCQNVSALPKG | |
| | FGVQELKILQSDLLFEQSRSAVQAKRADSPGRLVPCGAAISWSAV | |
| | PEQPLDVTANGFPQGTTKKTIGSLQARSQISKVELFRSFQKLLSRIA | |
| | RDKWPHSLRVQKLDTYQEYKEAASSYQEAWSTLRKQVFGSWIRN | |
| | PPDYHQFK | |
| Mus | MWTADEIAQLCYAHYNVRLPKQGKPEPNREWTLLAAVVKIQASA | SEQ |
| musculus | NQACDIPEKEVQVTKEVVSMGTGTKCIGQSKMRESGDILNDSHAE | ID |
| ADAT1 | IIARRSFQRYLLHQLHLAAVLKEDSIFVPGTQRGLWRLRPDLSFVF | NO: |
| | FSSHTPCGDASIIPMLEFEEQPCCPVIRSWANNSPVQETENLEDSKD | 854 |
| | KRNCEDPASPVAKKMRLGTPARSLSNCVAHHGTQESGPVKPDVS | |
| | SSDLTKEEPDAANGIASGSFRVVDVYRTGAKCVPGETGDLREPGA | |
| | AYHQVGLLRVKPGRGDRTCSMSCSDKMARWNVLGCQGALLMH | |
| | FLEKPIYLSAVVIGKCPYSQEAMRRALTGRCEETLVLPRGFGVQEL | |
| | EIQQSGLLFEQSRCAVHRKRGDSPGRLVPCGAAISWSAVPQQPLD | |
| | VTANGFPQGTTKKEIGSPRARSRISKVELFRSFQKLLSSIADDEQPD | |
| | SIRVTKKLDTYQEYKDAASAYQEAWGALRRIQPFASWIRNPPDYH | |
| | QFK | |
| Drosophila | MCDNKKPTVKEIAELCLKKFESLPKTGKPTANQWTILAGIVEFNR | SEQ |
| melancgaster | NTEACQLVSLGCGTKCIGESKLCPNGLILNDSHAEVLARRGFLRFL | ID |
| ADAT1 | YQELKQDRIFHWNSTLSTYDMDEHVEFHFLSTQTPCGDACILEEE | NO: |
| | QPAARAKRQRLDEDSEMVYTGAKLISDLSDDPMLQTPGALRTKP | 855 |
| | GRGERTLSMSCSDKIARWNVIGVQGALLDVLISKPIYFSSLNFCCD | |
| | DAQLESLERAIFKRFDCRTFKHTRFQPQRPQINIDPGIRFEFSQRSD | |
| | WQPSPNGLIWSQVPEELRPYEISVNGKRQGVTKKKMKTSQAALAI | |
| | SKYKLFLTFLELVKFNPKLSEMFDQQLSDPERIAYASCKDLARDY | |
| | QFAWREIKEKYFLQWTKKPHELLDFNPMSNK | |
| Xenopus | MQAKGLWSADEIAALSYGHYTTQLPKQGLPDPSREWTLMAAVIQ | SEQ |
| tropicalis | IESVEDTKVIKKVVAMGTGTKCIGQAKLRKTGDVLQDSHAEIIAK | ID |
| ADAT1 | RSFQRYLLHQLSLAVSDTKDCLFIPGTEKGKWMLRPEISFVFFTSH | NO: |
| | TPCGDASIIPVISHEDELGHPLPSEVTEKDHSSNNVCESVNTTYKRK | 856 |
| | VRSEEDIGFISKKMKHSIDEILTRPENYEEENRHDFPSTCQKALDVH | |
| | RTGAKCVAGELQDSYSPGVNYHTVGVLRIKPGRGDRTMSMSCSD | |
| | KMARWNVLGCQGALLMHFLQQPIYLSAVVVGKCPFSQDAMERA | |
| | LYNRCHKVLSLPCAFRLNRVQIIQSDLEFQHGRHALTKKDATRKL | |
| | VPCGAAVSWSAVPHHPLDVTANGYRQGTTRKAIGSPQCRSRICKA | |
| | EIFNTFRELVQRLSEKQRSESLSSQGLKTYWDYKAAAITYQEAWN | |
| | CLRQQAFTSWIQTPRDFLMFS | |
| Dictyostelium | MSWKLDKQFSDKICNFSHDFFNKKLIKKGKPISGEWTVLATLVLV | SEQ |
| discoideum | VENTSSYEIKQVLSLGTGNRCLGKSSLSNQGDVLNDSHAEIICKRS | ID |
| ADATI | FQKFCYNEILNLLQSKYYNSILFNIEYHDSNNNNKDNDNNGSLPTI | NO: |
| | SIKKGHSLHFYVNQTPCGDCSIFPFKKETQPENFIEKEKLEKDGKD | 857 |
| | KIENHEKKEQKDIIKQVDKDKDEENYEDEESKRKLKKVKDDNNN | |
| | NNNNNNNNNNNNNNNNNNNNNNNNNNNNNNNNNNINNNNNQ | |
| | YDDIQRTGAKTVFGEPEDKKLIGVDYHQIGVLRVKPGRGDPTVSM | |
| | SCSDKIARWNVLGIQGSLLSHFIKEQIFLSSITIGDLFNHSSIYRGLIG | |
| | RLLPNPTTTTTETSSSSSSSSSSSNTIPNFKLNSDLEIFSTNIQFQFSKL | |
| | LLESDQQNNNKSTSSGLAISFCYPNQHEVTIAINGKKMGTNQKNF | |
| | NAISQRSSICKFNLFKLFHQLVLIIKNKNSNEENEKNNQIVLIDSLFN | |
| | YYECKHLSKKYYQEYEKLKEFKFKNWLTNSSDLENFVLDN | |
| Schizosac | MAGDSVKSAIIGIAGGPFSGKTQLCEQLLERLKSSAPSTFSKLIHLT | SEQ |
| charomyces | SFLYPNSVDRYALSSYDIEAFKKVLSLISQGAEKICLPDGSCIKLPV | ID |
| pombe | DQNRIILIEGYYLLLPELLPYYTSKIFVYEDADTRLERCVLQRVKAE | NO: |
| ADAT2 | KGDLTKVLNDFVTLSKPAYDSSIHPTRENADIILPQKENIDTALLFV | 858 |
| | SQHLQDILAEMNKTSSSNTVKYDTQHETYMKLAHEILNLGPYFVI | |
| | QPRSPGSCVFVYKGEVIGRGFNETNCSLSGIRHAELIAIEKILEHYP | |
| | ASVFKETTLYVTVEPCLMCAAALKQLHIKAVYFGCGNDRFGGCG | |
| | SVFSINKDQSIDPSYPVYPGLFYSEAVMLMREFYVQENVKAPVPQ | |
| | SKKQRVLKREVKSLDLSRFK | |
| Saccharomyces | MQHIKHMRTAVRLARYALDHDETPVACIFVHTPTGQVMAYGMN | SEQ |
| cerevisiae | DTNKSLTGVAHAEFMGIDQIKAMLGSRGVVDVFKDITLYVTVEPC | ID |
| ADAT2 | IMCASALKQLDIGKVVFGCGNERFGGNGTVLSVNHDTCTLVPKN | NO: |
| | NSAAGYESIPGILRKEAIMLLRYFYVRQNERAPKPRSKSDRVLDKN | 859 |
| | TFPPMEWSKYLNEEAFIETFGDDYRTCFANKVDLSSNSVDWDLID | |
| | SHQDNIIQELEEQCKMFKFNVHKKSKV | |
| Xenopus | MTEEIQNWMHKAFQMAQDALNNGEVPVGCLMVYDNQVVGKGR | SEQ |
| tropicalis | NEVNETKNATRHAEMVAIDQVLDWCEKNSKKSRDVFENIVLYVT | ID |
| ADAT2 | VEPCIMCAGALRLLKIPLVVYGCRNERFGGCGSVLNVAGDNIPDT | NO: |
| | GTEFKYIGGYQAEKAVELLKTFYKQENPNAPRSKVRKKE | 860 |
| Bos taurus | MEAKAGPTAATDGAYSVSAEETEKWMEQAMQMAKDALDNTEV | SEQ |
| ADAT2 | PVGCLMVYNNEVVGKGRNEVNQTKNATRHAEMVAIDQALDWC | ID |
| | RRRGRSPSEVFEHTVLYVTVEPCIMCAAALRLMRIPLVVYGCQNE | |
| | RFGGCGSVLDIASADLPSTGKPFQCTPGYRAEEAVEMLKTFYKQE | NO: |
| | NPNAPKSKVRKKECHKS | 861 |
| Danio | MQEVGVDPEKNDFLQPSDSEVQTWMAKAFDMAVEALENGEVPV | SEQ |
| rerio | GCLMVYNNEIIGKGRNEVNETKNATRHAEMVALDQVLDWCRLR | ID |
| ADAT2 | EKDCKEVCEQTVLYVTVEPCIMCAAALRLLRIPFVVYGCKNERFG | NO: |
| | GCGSVLDVSSDHLPHTGTSFKCIAGYRAEEAVEMLKTFYKQENPN | 862 |
| | APKPKVRKDSINPQDGAAVIQVMRGPPDEETETIAHLS | |
| Arabidopsis | MEEDHCEDSHNYMGFALHQAKLALEALEVPVGCVFLEDGKVIAS | SEQ |
| thaliana | GRNRTNETRNATRHAEMEAIDQLVGQWQKDGLSPSQVAEKFSKC | ID |
| ADAT2 | VLYVTCEPCIMCASALSFLGIKEVYYGCPNDKFGGCGSILSLHLGS | NO: |
| | EEAQRGKGYKCRGGIMAEEAVSLFKCFYEQGNPNAPKPHRPVVQ | 863 |
| | RERT | |
| Mus | MEEKVESTTTPDGPCVVSVQETEKWMEEAMRMAKEALENIEVPV | SEQ |
| musculus | GCLMVYNNEVVGKGRNEVNQTKNATRHAEMVAIDQVLDWCHQ | ID |
| ADAT2 | HGQSPSTVFEHTVLYVTVEPCIMCAAALRLMKIPLVVYGCQNERF | NO: |
| | GGCGSVLNIASADLPNTGRPFQCIPGYRAEEAVELLKTFYKQENPN | 864 |
| | APKSKVRKKDCQKS | |
| Human | MEAKAAPKPAASGACSVSAEETEKWMEEAMHMAKEALENTEVP | SEQ |
| ADAT2 | VGCLMVYNNEVVGKGRNEVNQTKNATRHAEMVAIDQVLDWCR | ID |
| | QSGKSPSEVFEHTVLYVTVEPCIMCAAALRLMKIPLVVYGCQNER | NO: |
| | FGGCGSVLNIASADLPNTGRPFQCIPGYRAEEAVEMLKTFYKQEN | 865 |
| | PNAPKSKVRKKECQKS | |
| Arabidopsis | MDSDAWEIIHIPEKPSLSPDHQPTVKVYASLIKPRFANTIVRHLCKI | SEQ |
| thaliana | APLEDLRHVKRVKKKILPDCGETQLTVILCLAPEHNDQLSDMPPD | ID |
| ADAT3 | VQRLVDPYELSPFITQVCKYAAVSKEEWEEQSKIWPTSFHPPTYNI | NO: |
| | DGIGGFSEEETQSICKFMRVVIDMAVSGHTPLVNAAVIVDPSVRRII | 866 |
| | ASETDQVYASSAPRDMTSAETRPFEETGEICLNDTLEKQNGSLSAL | |
| | SCLNPWQWSLQPHDTENCSQWHPLRHASMVAIESSSARDRNLFP | |
| | NPSKIFDQDHVPPSNTDSPAKKQKTSSQSPDVQNDSREETVRDPS | |
| | MERPYLCTGYDIFLLLEPCTMCAMALVHQRIKRIFYAFPNTTAGG | |
| | LGSVHRLQGEKSLNHHYAVFRVLLPDDALRQMTTV | |
| Mus | MEPTSGFAEQPGPVKAESEEQEPAQWQALPVLSEQQSGAVELILA | SEQ |
| musculus | YAAPVLDKRQTSRLLREVSAVYPLPAQPHLKRVRPSRSAGGAQSS | ID |
| ADAT3 | DLLLCLAGPSAGPRSLAELLPRPAVDPRGLGTPFLVPVPARPPLTR | NO: |
| | SQFEEARAHWPTSFHEDKQVTSALAGQLFSTQERAAMQTHMERA | 867 |
| | VCAAQRAAAQGLRAVGAVVVDPASDRVLATGHDCSSVASPLLH | |
| | AVMVCIDLVAQGQGRGSCDLRSHPACSFTQATATQGARAGSVRK | |
| | LDEDSLPYVCTGYDLYVTREPCVMCAMALVHARIQRVFYGAPSP | |
| | DGALGTLFRVHARPDLNHRFQVFRGILEDQCRQLDPDP | |
| Danio | MEPQAKRKKEMDDYDDTWEVLPVLSDEQSQDPELLPAYAAPILE | SEQ |
| rerio | RRETSRLVKELSLIHPLPNLQHIKRVRPCKHKDSPHPLEVIVCLVSD | ID |
| ADAT3 | VQCTDPKKVTLSHLLHTQCFNSNGLGDPFIVQIPANPPLTRPQFEK | NO: |
| | ASKHWPTSFHEDKLVTFALKGQLFTAHQKTKMREYMCVAVKAA | 868 |
| | KSGRELGMDAVGAVIVDPKTEQIIAVAHDCKRGSHPLHHAVMVC | |
| | IDLVACGQDGGAYNYEKYPACRFSCSNSVCDGKETGLPYICTGYD | |
| | LYVTREPCVMCAMALVHSRISRVFYGASTADGAFGSRYKIHCQK | |
| | DLNHRFEVFKGVMVNACEDLCKE | |
| Schizosac | MVKTNISKNSPKEATVPELDWPFKLIKSHLETRKLETENVWIACFE | SEQ |
| charomyces | PKYASKVTQYVKQIRSKQKESLLHCNRLRRIQDENGSLELQIIICPE | ID |
| pombe | KSMTANEIGKDFEDLGIVSKMIFLYAVPAFPPLTDEQFHEWNSVW | NO: |
| ADAT3 | PVSYRKHVQRQDVFTVHELKRIESILEDLINAAGASHKHGEIGCAA | 869 |
| | AIYDPTTDTVLAVSVDERSKLKNPINHCVMNAINLVAKRELSRRQ | |
| | NRTDGSKDRYLCKDLTVVMTHEPCVMCSMGLLHSRIRRLIYCKK | |
| | QPLTGGIESLYGIHWRAELNHRYLAYSGWNKPVPSIKENIHV | |
| Saccharomyces | MVKKVNNPLKIDYQNGIIENRLLQIRNFKDVNTPKLINVWSIRIDP | SEQ |
| cerevisiae | RDSKKVIELIRNDFQKNDPVSLRHLKRIRKDIETSTLEVVLCSKEYI | ID |
| ADAT3 | CDEGEINNKLKSIWVGTKKYELSDDIEVPEFAPSTKELNNAWSVK | NO: |
| | YWPLIWNGNPNDQILNDYKIDMQEVRNELSRASTLSVKMATAGK | 870 |
| | QFPMVSVFVDPSRKKDKVVAEDGRNCENSLPIDHSVMVGIRAVG | |
| | ERLREGVDEDANSYLCLDYDVYLTHEPCSMCSMALIHSRVRRVV | |
| | FLTEMQRTGSLKLTSGDGYCMNDNKQLNSTYEAFQWIGEEYPVG | |
| | QVDRDVCC | |
| Human | MEPAPGLVEQPKCLEAGSPEPEPAPWQALPVLSEKQSGDVELVLA | SEQ |
| ADAT3 | YAAPVLDKRQTSRLLKEVSALHPLPAQPHLKRVRPSRDAGSPHAL | ID |
| | EMLLCLAGPASGPRSLAELLPRPAVDPRGLGQPFLVPVPARPPLTR | NO: |
| | GQFEEARAHWPTSFHEDKQVTSALAGRLFSTQERAAMQSHMERA | 871 |
| | VWAARRAAARGLRAVGAVVVDPASDRVLATGHDCSCADNPLLH | |
| | AVMVCVDLVARGQGRGTYDFRPFPACSFAPAAAPQAVRAGAVR | |
| | KLDADEDGLPYLCTGYDLYVTREPCAMCAMALVHARILRVFYGA | |
| | PSPDGALGTRFRIHARPDLNHRFQVFRGVLEEQCRWLDPDT | |
| Rattus | MEPTSGFAEQPGPEKVESEEQEPAQWQALPVLSEQQSGAVELVLA | SEQ |
| norvegicus | YAAPVLDKRQTSRLLREVSAVYPLPAQPHLKRVRPSRSAGGAHSS | ID |
| ADAT3 | DLLLCLAGPSAGPRSLAELLPRPAVDPRGLGTPFLVPVPARPPLTR | NO: |
| | SQFEEARAHWPTSFHEDKQVTSALAGQLFSAQARAAMQTHMER | 872 |
| | AVRAAQRAAAQGLRAVGAVVVDPASDHVLATGHDCCSEASPLL | |
| | HAVMVCIDLVAQGQGRGSCDLRRHPACSFTQATATQSARAGSVR | |
| | KLDEDSLPYVCTGYDLYVTREPCVMCAMALVHARIQRVFYGAPS | |
| | PDGALGTRFRVHARPDLNHRFQVFRGILEDQCRQLDPDP | |
| Rattus | MDIEDEENMSSSSIDVKENRNLDNMPPKDSSTPGPGEGIPLSNGGG | SEQ |
| norvegicus | GSTSRKRPLEEGSNGHSKYRLKKRRKTPGPVLPKNALMQLNEIKP | ID |
| ADARB1 | GLQYMLLSQTGPVHAPLFVMSVEVNGQVFEGSGPTKKKAKLHAA | NO: |
| | EKALRSFVQFPNASEAHLAMGRTLSVNTDFTSDQADFPDTLFNGF | 873 |
| | ETPDKSEPPFYVGSNGDDSFSSSGDVSLSASPVPASLTQPPLPIPPPF | |
| | PPPSGKNPVMILNELRPGLKYDFLSESGESHAKSFVMSVVVDGQF | |
| | FEGSGRNKKLAKARAAQSALATVFNLHLDQTPSRQPVLSEGLQLH | |
| | LPQVLADAVSRLVLGKFSDLTDNFSSPHARRKVLSGVVMTTGTD | |
| | VKDAKVISVSTGTKCINGEYMSDRGLALNDCHAEIISRRSLLRFLY | |
| | AQLELYLNNKEDQKKSIFQKSERGGFRLKDTVQFHLYISTSPCGD | |
| | ARIFSPHEPVLEGMAPDSHQLTEPADRHPNRKARGQLRTKIESGEG | |
| | TIPVRSNASIQTWDGVLQGERLLTMSCSDKIARWNVVGIQGALLSI | |
| | FVEPIYFSSIILGSLYHGDHLSRAMYQRISNIEDLPPLYTLNKPLLSG | |
| | ISNAEARQPGKAPNFSVNWTVGDTAIEVINATTGKDELGRPSRLC | |
| | KHALYCRWMRVHGKVPPHLLRTKITKPTTYHESKLAAKEYQAAK | |
| | ARLFTAFIKAGLGAWVEKPTEQDQFSFTP | |
| Human | MNPRQGYSLSGYYTHPFQGYEHRQLRYQQPGPGSSPSSFLLKQIEF | SEQ |
| ADAR | LKGQLPEAPVIGKQTPSLPPSLPGLRPRFPVLLASSTRGRQVDIRGV | ID |
| | PRGVHLRSQGLQRGFQHPSPRGRSLPQRGVDCLSSHFQELSIYQDQ | NO: |
| | EQRILKFLEELGEGKATTAHDLSGKLGTPKKEINRVLYSLAKKGK | 874 |
| | LQKEAGTPPLWKIAVSTQAWNQHSGVVRPDGHSQGAPNSDPSLE | |
| | PEDRNSTSVSEDLLEPFIAVSAQAWNQHSGVVRPDSHSQGSPNSDP | |
| | GLEPEDSNSTSALEDPLEFLDMAEIKEKICDYLFNVSDSSALNLAK | |
| | NIGLTKARDINAVLIDMERQGDVYRQGTTPPIWHLTDKKRERMQI | |
| | KRNTNSVPETAPAAIPETKRNAEFLTCNIPTSNASNNMVTTEKVEN | |
| | GQEPVIKLENRQEARPEPARLKPPVHYNGPSKAGYVDFENGQWA | |
| | TDDIPDDLNSIRAAPGEFRAIMEMPSFYSHGLPRCSPYKKLTECQL | |
| | KNPISGLLEYAQFASQTCEFNMIEQSGPPHEPRFKFQVVINGREFPP | |
| | AEAGSKKVAKQDAAMKAMTILLEEAKAKDSGKSEESSHYSTEKE | |
| | SEKTAESQTPTPSATSFFSGKSPVTTLLECMHKLGNSCEFRLLSKE | |
| | GPAHEPKFQYCVAVGAQTFPSVSAPSKKVAKQMAAEEAMKALH | |
| | GEATNSMASDNQPEGMISESLDNLESMMPNKVRKIGELVRYLNT | |
| | NPVGGLLEYARSHGFAAEFKLVDQSGPPHEPKFVYQAKVGGRWF | |
| | PAVCAHSKKQGKQEAADAALRVLIGENEKAERMGFTEVTPVTGA | |
| | SLRRTMLLLSRSPEAQPKTLPLTGSTFHDQIAMLSHRCFNTLTNSF | |
| | QPSLLGRKILAAIIMKKDSEDMGVVVSLGTGNRCVKGDSLSLKGE | |
| | TVNDCHAEIISRRGFIRFLYSELMKYNSQTAKDSIFEPAKGGEKLQI | |
| | KKTVSFHLYISTAPCGDGALFDKSCSDRAMESTESRHYPVFENPK | |
| | QGKLRTKVENGEGTIPVESSDIVPTWDGIRLGERLRTMSCSDKILR | |
| | WNVLGLQGALLTHFLQPIYLKSVTLGYLFSQGHLTRAICCRVTRD | |
| | GSAFEDGLRHPFIVNHPKVGRVSIYDSKRQSGKTKETSVNWCLAD | |
| | GYDLEILDGTRGTVDGPRNELSRVSKKNIFLLFKKLCSFRYRRDLL | |
| | RLSYGEAKKAARDYETAKNYFKKGLKDMGYGNWISKPQEEKNF | |
| | YLCPV | |
| Rattus | MSQGFRGPTGVFPHQTQPCLDPSYEHSKWRYLQPRGSESYLRSFQ | SEQ |
| norvegicus | LQQIEFLKGRLPEAPLIGAQTQSLPPFLPGHWPRFPGPPAQGKQPEI | ID |
| ADAR | WGFPRSVTLRNQGFHIGPPLPPPHSRGPPWRGAEGLCSHFQELSIS | NO: |
| | QNPEQKVLNRLEELGEGKATTAYALARELRTPKKDINRILYSLER | 875 |
| | KGKLHRGVGKPPLWSLVPLSQACTQPPRAVNSDKEVPRGEPDLDS | |
| | EDGDPASDLEGPSELLDMAEIKEKICDYLFNVSKSSALNLAKNIGL | |
| | AKARDVNAVLIDLERQGDVYREGATPPIWYLTDKKRERLQMKRS | |
| | THSGPAATPAAVSEATQSTSFPTCHPPQSGGSSSMATSKRVENGQE | |
| | PVTKYESRHEARPGPVRLRPHAYHNGPSRAGYVASENGPWATDD | |
| | IPDNLNSIHTAPGEFRAIMEMPSFYSPTLPRCSPYKKLTECQLKNPV | |
| | SGLLEYAQFTSQTCDFNLIEQSGPSHEPRFKFQVVINGREFPPAEAG | |
| | SKKVAKQDAAVKAMAILLREAKAKDSGQPEELSNCPMEEDPEKP | |
| | AESQPPSSSATSLFSGKSPVTTLLECMHKLGNSCEFRLLSKEGPAH | |
| | DPKFQYCVAVGAQTFPSVSAPSKKVAKQMAAEEAMKALQEEAA | |
| | NSADDQSGGANTDSLDESVAPNKIRRIGELVRYLNTNPVGGLLEY | |
| | ARSHGFAAEFKLIDQSGPPHEPKFVYQAKVGGRWFPAVCAHSKK | |
| | QGKQDAADAALRVLIGESEKAEQLGFAEVTPVTGASLRRTMLLLS | |
| | RSPDAHPKTLPLTGSTFHDQIAMLSHRCFNALTNSFQPSLLGRKIL | |
| | AAIIMKRDPEDMGVVVSLGTGNRCVKGDSLSLKGETVNDCHAEII | |
| | SRRGFIRFLYSELMKYNHHTAKNSIFELARGGEKLQIKKTVSFHLY | |
| | ISTAPCGDGAHFDKSCSDRAVESTESRHYPVFENPKQGKLRTKVE | |
| | NGEGTIPVESSDIVPTWDGIRLGERLRTMSCSDKILRWNVLGLQGA | |
| | LLTHFLQPVYLKSVTLGYLFSQGHLTRAICCRVTRDGNAFEDGLR | |
| | YPFIVNHPKVGRVSVYDSKRQSGKTKETSVNWCLADGYDLEILD | |
| | GTRGTVDGPGKELSRVSKKNIFLQFKKLCSFRARRDLLQLSYGEA | |
| | KKAARDYDLAKNYFKKSLRDMGYGNWISKPQEEKNFYLCPVPN | |
| | D | |
| Human | MDIEDEENMSSSSTDVKENRNLDNVSPKDGSTPGPGEGSQLSNGG | SEQ |
| ADARB1 | GGGPGRKRPLEEGSNGHSKYRLKKRRKTPGPVLPKNALMQLNEI | ID |
| | KPGLQYTLLSQTGPVHAPLFVMSVEVNGQVFEGSGPTKKKAKLH | NO: |
| | AAEKALRSFVQFPNASEAHLAMGRTLSVNTDFTSDQADFPDTLFN | 876 |
| | GFETPDKAEPPFYVGSNGDDSFSSSGDLSLSASPVPASLAQPPLPVL | |
| | PPFPPPSGKNPVMILNELRPGLKYDFLSESGESHAKSFVMSVVVDG | |
| | QFFEGSGRNKKLAKARAAQSALAAIFNLHLDQTPSRQPIPSEGLQL | |
| | HLPQVLADAVSRLVLGKFGDLTDNFSSPHARRKVLAGVVMTTGT | |
| | DVKDAKVISVSTGTKCINGEYMSDRGLALNDCHAEIISRRSLLRFL | |
| | YTQLELYLNNKDDQKRSIFQKSERGGFRLKENVQFHLYISTSPCGD | |
| | ARIFSPHEPILEGSRSYTQAGVQWCNHGSLQPRPPGLLSDPSTSTFQ | |
| | GAGTTEPADRHPNRKARGQLRTKIESGEGTIPVRSNASIQTWDGV | |
| | LQGERLLTMSCSDKIARWNVVGIQGSLLSIFVEPIYFSSIILGSLYHG | |
| | DHLSRAMYQRISNIEDLPPLYTLNKPLLSGISNAEARQPGKAPNFS | |
| | VNWTVGDSAIEVINATTGKDELGRASRLCKHALYCRWMRVHGK | |
| | VPSHLLRSKITKPNVYHESKLAAKEYQAAKARLFTAFIKAGLGAW | |
| | VEKPTEQDQFSLTP | |
| Mus | MDIEDEENMSSSSTDIKENRNLDNMPPKDSSTPGPGEGIPLSNGGG | SEQ |
| musculus | GSTSRKRPLEEGSNGHSKYRLKKRRKTPGPVLPKNALMQLNEIKP | ID |
| ADARB1 | GLQYMLLSQTGPVHAPLFVMSVEVNGQVFEGSGPTKKKAKLHAA | NO: |
| | EKALRSFVQFPNASEAHLAMGRTLSVNTDFTSDQADFPDTLFNGF | 877 |
| | ETPDKSEPPFYVGSNGDDSFSSSGDVSLSASPVPASLTQPPLPIPPPF | |
| | PPPSGKNPVMILNELRPGLKYDELSESGESHAKSFVMSVVVDGQF | |
| | FEGSGRNKKLAKARAAQSALATVFNLHLDQTPSRQPVLSEGLQLH | |
| | LPQVLADAVSRLVLGKFSDLTDNFSSPHARRKVLSGVVMTTGTD | |
| | VKDAKVISVSTGTKCINGEYMSDRGLALNDCHAEIISRRSLLRFLY | |
| | AQLELYLNNKEDQKKSIFQKSERGGFRLKDTVQFHLYISTSPCGD | |
| | ARIFSPHEPVLEGMTPDSHQLTEPADRHPNRKARGQLRTKIESGEG | |
| | TIPVRSNASIQTWDGVLQGERLLTMSCSDKIARWNVVGIQGSLLSI | |
| | FVEPIYFSSIILGSLYHGDHLSRAMYQRISNIEDLPPLYTLNKPLLSG | |
| | ISNAEARQPGKAPNFSVNWTVGDATIEVINATTGKDELGRPSRLC | |
| | KHALYCRWMRVHGKVPPHLLRTKITKPTTYHESKLAAREYQAAK | |
| | ARLFTAFIKAGLGAWVEKPTEQDQFSFTP | |
| Mus | MSQGFRGPTGVFPHQTQSYLDPSHEHSKWRYPQPQGPESYPRSFQ | SEQ |
| musculus | LQQIEFLKGRLPEAPLIGIQTQSLPPFLPGHWPRFPGPPAQDRQLEI | ID |
| ADAR | WEFPRSVTLRNQGFHIGPPLPPPHSRGTPWRGADGLCSHFRELSIS | NO: |
| | QSPEQKVLNRLEELGEGKATTAHVLARELRIPKRDINRILYSLEKK | 878 |
| | GKLHRGRGKPPLWSLVPLSQAWTQPPGVVNPDSCIQEFPRGEPGL | |
| | DSEDGDPASDLEGPSEPLDMAEIKEKICDYLFNVSNSSALNLAKNI | |
| | GLTKARDVTSVLIDLERQGDVYRQGATPPIWYLTDKKRERLQMK | |
| | RSTHSAPAPTPTAVPEATRSPSFPACHPPPAGASSSVAASKRVENG | |
| | QEPAIKHESRHEARPGPMRLRPHAYHNGPSRAGYVASENGQWAT | |
| | DDIPDNLNSIHTAPGEFRAIMEMPSFYSPTLPRCSPYKKLTECQLKN | |
| | PVSGLLEYAQFTSQTCDFNLIEQSGPSHEPRFKFQVVINGREFPPAE | |
| | AGSKKVAKQDAAVKAMAILLREAKAKDSGQPEDLSHCPMEEDSE | |
| | KPAEAQAPSSSATSLFSGKSPVTTLLECMHKLGNSCEFRLLSKEGP | |
| | AHDPKFQYCVAVGAQTFPPVSAPSKKVAKQMAAEEAMKALQEE | |
| | AASSADDQSGGANTDSLDESMAPNKIRRIGELVRYLNTNPVGGLL | |
| | EYARSHGFAAEFKLIDQSGPPHEPKFVYQAKVGGRWFPAVCAHS | |
| | KKQGKQDAADAALRVLIGESEKAEQLGFAEVTPVTGASLRRTML | |
| | LLSRSPDAHPKTLPLSGSTFHDQIAMLSHRCFNALTNSFQPSLLGR | |
| | KILAAIIMKRDPEDMGVVVSLGTGNRCVKGDSLSLKGETVNDCH | |
| | AEIISRRGFIRFLYSELMKYNHHTAKNSIFELARGGEKLQIKKTVSF | |
| | HLYISTAPCGDGALFDKSCSDRAVESTESRHYPVFENPKQGKLRT | |
| | KVENGEGTIPVESSDIVPTWDGIRLGERLRTMSCSDKILRWNVLGL | |
| | QGALLTHFLQPVYLKSVTLGYLFSQGHLTRAICCRVTRDGKAFED | |
| | GLRYPFIVNHPKVGRVSVYDSKRQSGKTKETSVNWCMADGYDLE | |
| | ILDGTRGTVDGPGKELSRVSKKNIFLQFKKLCSFRARRDLLQLSYG | |
| | EAKKAARDYDLAKNYFKKSLRDMGYGNWISKPQEEKNFYLCPVP | |
| | ND | |
| Human | MKQLFPPPPGTSLTHALGAWRGRERAQAATSLLASSASQFPTAVE | SEQ |
| ADARB2- | DALMSVLTSHCAPSTPAATRAQQTGTRGHIHPACPCQQSCVGASR | ID |
| AS1 | PPGRPQIFLPLTTALSLEAYAADTCSAADFLHNPSSWGKVWYLNE | NO: |
| | ASFDLYSYHYFW | 879 |
| Rattus | MASVLGSGRGSGGLSSQLKCKSKRRRRRRSKRKDKVSILSTFLAP | SEQ |
| norvegicus | FKYLSPGTTNTEDEDNLSTSSAEVKENRNVSNLGTRPLPPGDWAR | ID |
| ADARB2 | GGSTPSVKRKRPLEEGNGGHFCKLQLIWKKLSWSMTPKNALVQL | NO: |
| | HELKPGLQYRMVSQTGPVHAPVFAVAVEVNGLTFEGTGPTKKKA | 880 |
| | KMRAAEMALKSFVQFPNAFQAHLAMGSSTSPCTDFTSDQADFPD | |
| | TLFKEFEPSSRNEDFPGCCPVDTEFLSSAYRRGRLLYHTLDLMGQA | |
| | LPDRSRLAPGALGERNPVVVLNELRSGLRYVCLSETAEKPRVKSF | |
| | VMAVCVDGRTFEGSGRSKKLAKGQAAQAALQALFDIRLPGHIPSR | |
| | SKSNLLPQDFADSVSQLVTQKFRELTVGLTSVYARHKTLAGIVMT | |
| | KGLDTKQAQVIVLSSGTKCISGEHISDQGLVVNDCHAEIVARRAFL | |
| | HFLYTQLELHLSKHQEDPERSIFIRVKEGGYRLRENILFHLYVSTSP | |
| | CGDARLNSPYEITIDLNSSKHIVRKFRGHLRTKIESGEGTVPVRGPS | |
| | AVQTWDGILLGEQLVTMSCTDKIASWNVLGLQGALLCHFIEPVYL | |
| | HSIIVGSLHHTGHLARVMSHRMEGIGQLPASYRQNRPLLSGVSNA | |
| | EARQPGKSPHFSANWVVGSADLEIINATTGKRSCGGSSRLCKHVF | |
| | SARWARLHGRLSTRIPGHGDTPSMYCEAKRGAHTYQSVKQQLFK | |
| | AFQKAGLGTWVRKPPEQDQFLLSL | |
| Mus | MASVLGSGRGSGGLSSQLKCKSKRRRRRRSKRKDKVSILSTFLAP | SEQ |
| musculus | FKYLSPGTTNTEDEDNLSTSSAEVKENRNVSNLGTRPLPPGDWAR | ID |
| ADARB2 | GSTPSVKRKRPLEEGNGGHFCKLQLIWKKLSWSVTPKNALVQLH | NO: |
| | ELKPGLQYRMVSQTGPVHAPVFAVAVEVNGLTFEGTGPTKKKAK | 881 |
| | MRAAEMALKSFVQFPNAFQAHLAMGSSTSPCTDFTSDQADFPDT | |
| | LFKEFEPSSKNEDFPGCHPVDTEFLSSAYRRGRLLYHTLDLMGQA | |
| | LPDRSRLAPGALGERNPVVVLNELRSGLRYVCLSETAEKPRVKSF | |
| | VMAVCVDGRTFEGSGRSKKLAKGQAAQAALQALFDIRLPGHIPSR | |
| | SKSNLLPQDFADSVSQLVTQKFRELTVGLTSVYARHKTLAGIVMT | |
| | KGLDTKQAQVIVLSSGTKCISGEHISDQGLVVNDCHAEIVARRAFL | |
| | HFLYSQLELHLSKHQEDPERSIFIRLKEGGYRLRENILFHLYVSTSP | |
| | CGDARVNSPYEITTDLNSSKHIVRKFRGHLRTKIESGEGTVPVRGP | |
| | SAVQTWDGILLGEQLITMSCTDKIASWNVLGLQGALLCHFIEPVY | |
| | LHSIIVGSLHHTGHLARVMSHRMEGIGQLPASYRQNRPLLSGVSH | |
| | AEARQPGKSPHFSANWVVGSADLEIINATTGKRSCGGSSRLCKHV | |
| | FSAWWARLHGRLSTRIPSHGDTPSMYCEAKQGAHTYQSVKQQLF | |
| | KAFQKAGLGTWVRKPPEQDQFLLSL | |
| Human | MASVLGSGRGSGGLSSQLKCKSKRRRRRRSKRKDKVSILSTFLAP | SEQ |
| ADARB2 | FKHLSPGITNTEDDDTLSTSSAEVKENRNVGNLAARPPPSGDRAR | ID |
| | GGAPGAKRKRPLEEGNGGHLCKLQLVWKKLSWSVAPKNALVQL | NO: |
| | HELRPGLQYRTVSQTGPVHAPVFAVAVEVNGLTFEGTGPTKKKA | 882 |
| | KMRAAELALRSFVQFPNACQAHLAMGGGPGPGTDFTSDQADFPD | |
| | TLFQEFEPPAPRPGLAGGRPGDAALLSAAYGRRRLLCRALDLVGP | |
| | TPATPAAPGERNPVVLLNRLRAGLRYVCLAEPAERRARSFVMAVS | |
| | VDGRTFEGSGRSKKLARGQAAQAALQELFDIQMPGHAPGRARRT | |
| | PMPQEFADSISQLVTQKFREVTTDLTPMHARHKALAGIVMTKGLD | |
| | ARQAQVVALSSGTKCISGEHLSDQGLVVNDCHAEVVARRAFLHF | |
| | LYTQLELHLSKRREDSERSIFVRLKEGGYRLRENILFHLYVSTSPCG | |
| | DARLHSPYEITTDLHSSKHLVRKFRGHLRTKIESGEGTVPVRGPSA | |
| | VQTWDGVLLGEQLITMSCTDKIARWNVLGLQGALLSHFVEPVYL | |
| | QSIVVGSLHHTGHLARVMSHRMEGVGQLPASYRHNRPLLSGVSD | |
| | AEARQPGKSPPFSMNWVVGSADLEIINATTGRRSCGGPSRLCKHV | |
| | LSARWARLYGRLSTRTPSPGDTPSMYCEAKLGAHTYQSVKQQLF | |
| | KAFQKAGLGTWVRKPPEQQQFLLTL | |
| Rattus | MPLFKLTGQGKQIDDAMRSFAEKVFASEVKDEGGRHEISPFDVDE | SEQ |
| norvegicus | ICPISLREMQAHIFHMENLSMSMDGRRKRRFQGRKTVNLSIPQSET | ID |
| AMPD1 | SSTKLSHIEEFISSSPTYESVPDFQRVQITGDYASGVTVEDFEVVCK | NO: |
| | GLYRALCIREKYMQKSFQRFPKTPSKYLRNIDGEALVAIESFYPVF | 883 |
| | TPPPKKGEDPFRREDLPANLGYHLKMKGGVIYIYPDEAAASRDEP | |
| | KPYPYPNLDDFLDDMNFLLALIAQGPVKTYTHRRLKFLSSKFQVH | |
| | QMLNEMDELKELKNNPHRDFYNCRKVDTHIHAAACMNQKHLLR | |
| | FIKKSYHIDADRVVYSTKEKNLTLKELFAQLNMHPYDLTVDSLDV | |
| | HAGRQTFQRFDKFNDKYNPVGASELRDLYLKTDNYINGEYFATII | |
| | KEVGADLVDAKYQHAEPRLSIYGRSPDEWSKLSSWFVGNRIYCPN | |
| | MTWMIQVPRIYDVFRSKNFLPHFGKMLENIFLPVFEATINPQTHPD | |
| | LSVFLKHITGFDSVDDESKHSGHMFSSKSPKPEEWTMENNPSYTY | |
| | YAYYMYANIMVLNCLRKERGMNTFLFRPHCGEAGALTHLMTAF | |
| | MIADNISHGLNLKKSPVLQYLFFLAQIPIAMSPLSNNSLFLEYAKNP | |
| | FLDFLQKGLMISLSTDDPMQFHFTKEPLMEEYAIAAQVFKLSTCD | |
| | MCEVARNSVLQCGISHEEKAKFLGNNYLEEGPVGNDIRRTNVAQI | |
| | RMAYRYETWCYELNLIAEGLKSTE | |
| Human | MPLFKLPAEEKQIDDAMRNFAEKVFASEVKDEGGRQEISPFDVDEI | SEQ |
| AMPD1 | CPISHHEMQAHIFHLETLSTSTEARRKKRFQGRKTVNLSIPLSETSS | ID |
| | TKLSHIDEYISSSPTYQTVPDFQRVQITGDYASGVTVEDFEIVCKGL | NO: |
| | YRALCIREKYMQKSFQRFPKTPSKYLRNIDGEAWVANESFYPVFT | 884 |
| | PPVKKGEDPFRTDNLPENLGYHLKMKDGVVYVYPNEAAVSKDEP | |
| | KPLPYPNLDTFLDDMNFLLALIAQGPVKTYTHRRLKFLSSKFQVH | |
| | QMLNEMDELKELKNNPHRDFYNCRKVDTHIHAAACMNQKHLLR | |
| | FIKKSYQIDADRVVYSTKEKNLTLKELFAKLKMHPYDLTVDSLDV | |
| | HAGRQTFQRFDKFNDKYNPVGASELRDLYLKTDNYINGEYFATII | |
| | KEVGADLVEAKYQHAEPRLSIYGRSPDEWSKLSSWFVCNRIHCPN | |
| | MTWMIQVPRIYDVFRSKNFLPHFGKMLENIFMPVFEATINPQADP | |
| | ELSVFLKHITGFDSVDDESKHSGHMFSSKSPKPQEWTLEKNPSYTY | |
| | YAYYMYANIMVLNSLRKERGMNTFLFRPHCGEAGALTHLMTAF | |
| | MIADDISHGLNLKKSPVLQYLFFLAQIPIAMSPLSNNSLFLEYAKNP | |
| | FLDFLQKGLMISLSTDDPMQFHFTKEPLMEEYAIAAQVFKLSTCD | |
| | MCEVARNSVLQCGISHEEKVKFLGDNYLEEGPAGNDIRRTNVAQI | |
| | RMAYRYETWCYELNLIAEGLKSTE | |
| Oryctolagus | MNQKHLLRFIKKSYQVDADRVVYSTK | SEQ |
| cuniculus | | ID |
| AMPD1 | | NO: |
| | | 885 |
| Gallus | MNQKHLLRFIKKSYRVDADRVVYDAK | SEQ |
| gallus | | ID |
| AMPD1 | | NO: |
| | | 886 |
| Mus | MPLFKLTGQGKQIDDAMRSFAEKVFASEVKDEGGRHEISPFDVDE | SEQ |
| musculus | ICPISLHEMQAHIFHMENLSMDGRRKRRFQGRKTVNLSIPQSETSS | ID |
| AMPD1 | TKLSHIEEFISSSPTYESVPDFQRVQITGDYASGVTVEDFEVVCKGL | NO: |
| | YRALCIREKYMQKSFQRFPKTPSKYLRNIDGEALVGNESFYPVFTP | 887 |
| | PPKKGEDPFRTEDLPANLGYHLKMKAGVIYIYPDEAAANRDEPKP | |
| | YPYPNLDDFLDDMNFLLALIAQGPVKTYAHRRLKFLSSKFQVHQ | |
| | MLNEMDELKELKNNPHRDFYNCRKVDTHIHAAACMNQKHLLRFI | |
| | KKSYHIDADRVVYSTKEKSLTLKELFAKLNMHPYDLTVDSLDVH | |
| | AGRQTFQRFDKFNDKYNPVGASELRDLYLKTDNYINGEYFATIIK | |
| | EVGADLVEAKYQHAEPRLSIYGRSPDEWNKLSSWFVCNRIYCPN | |
| | MTWMIQVPRIYDVFRSKNFLPHFGKMLENIFLPVFEATINPQAHPD | |
| | LSVFLKHITGFDSVDDESKHSGHMFSSKSPKPEEWTMENNPSYTY | |
| | YAYYMYANITVLNSLRKERGMNTFLFRPHCGEAGALTHLMTAFM | |
| | IADNISHGLNLKKSPVLQYLFFLAQIPIAMSPLSNNSLFLEYAKNPF | |
| | LDFLQKGLMISLSTDDPMQFHFTKEPLMEEYAIAAQVFKLSTCDM | |
| | CEVARNSVLQCGISHEEKAKFLGNNYLEEGPVGNDIRRTNVAQIR | |
| | MAYRYETWCYELNLIAEGLKATE | |
| Dictyostelium | MSTPLRGSSPQVSFYESELDQEGGSDASHFTYRNYMEDDKINSFTF | SEQ |
| discoideum | NMARKDQTQLFQRIILTNESESEIEEYAEVAEQLLDAINLREKYVF | ID |
| AMPD1 | HPKIWKADAPVGEKPPYSPFESDESTNCATEHMFKEVNGVYFVYS | NO: |
| | NETDMKSNKALFSVPHTLASYYKDINNLMMLSSYGPAKTFTFKRL | 888 |
| | QLLESKFNMHTLLNDSLELFQQKTAPHRDFYNVRKVDTHVHHSS | |
| | SMNQKHLLKFIKRKLKENPNEIVIFRDDKYLTLAEVFKSLNLDVDE | |
| | LSVDTLDVHADNNTFHRFDKFNLKYNPCGQSRLREIFLKTDNLIK | |
| | GKYLAEISKEVFTDLESSKYQCAEYRLSIYGRKMSEWDTLASWIV | |
| | DNDLFSTKVRWLIQVPRLYDVYRETSTTTFQDFLNNVFHPLFEVT | |
| | KDPSSHPKLHLFLQQVVGIDCVDDESKFEKKFTEKFPVPGEWSSE | |
| | HNPPYTYYLYYLYANLYTLNQFREEKGLNILTLRPHSGEAGEVDH | |
| | MGAAFYLAHGINHGINLRKTPVLQYLYYLTQIGIAMSPLSNNSLFL | |
| | TYNRNPFPAFFARGLNVSISTDDPLQFHYTKEPLMEEYSIATQVWR | |
| | LSVCDICEIARNSVLQSGFEHNVKSHWLGPDYANSGGNDIKKTNIS | |
| | DIRVCFRNETLIEELHLILKSLQTLPNFKNLNINFLLDKLPSEITTGN | |
| | DYKLKKAQLKLNGANKLRNSSVGSTPNNGTPSSSGTPSLSSPGAIV | |
| | HLMKTKPYIPPPLSLNIKQENNNNNNNNNNNNNNNNNNNTNTNT | |
| | NSNSTTTNQDDNSKSDK | |
| Human | MPRQFPKLNISEVDEQVRLLAEKVFAKVLREEDSKDALSLFTVPE | SEQ |
| AMPD3 | DCPIGQKEAKERELQKELAEQKSVETAKRKKSFKMIRSQSLSLQM | ID |
| | PPQQDWKGPPAASPAMSPTTPVVTGATSLPTPAPYAMPEFQRVTIS | NO: |
| | GDYCAGITLEDYEQAAKSLAKALMIREKYARLAYHRFPRITSQYL | 889 |
| | GHPRADTAPPEEGLPDFHPPPLPQEDPYCLDDAPPNLDYLVHMQG | |
| | GILFVYDNKKMLEHQEPHSLPYPDLETYTVDMSHILALITDGPTKT | |
| | YCHRRLNFLESKFSLHEMLNEMSEFKELKSNPHRDFYNVRKVDT | |
| | HIHAAACMNQKHLLRFIKHTYQTEPDRTVAEKRGRKITLRQVFDG | |
| | LHMDPYDLTVDSLDVHAGRQTFHRFDKFNSKYNPVGASELRDLY | |
| | LKTENYLGGEYFARMVKEVARELEESKYQYSEPRLSIYGRSPEEW | |
| | PNLAYWFIQHKVYSPNMRWIIQVPRIYDIFRSKKLLPNFGKMLENI | |
| | FLPLFKATINPQDHRELHLFLKYVTGFDSVDDESKHSDHMFSDKSP | |
| | NPDVWTSEQNPPYSYYLYYMYANIMVLNNLRRERGLSTFLFRPH | |
| | CGEAGSITHLVSAFLTADNISHGLLLKKSPVLQYLYYLAQIPIAMSP | |
| | LSNNSLFLEYSKNPLREFLHKGLHVSLSTDDPMQFHYTKEALMEE | |
| | YAIAAQVWKLSTCDLCEIARNSVLQSGLSHQEKQKFLGQNYYKE | |
| | GPEGNDIRKTNVAQIRMAFRYETLCNELSFLSDAMKSEEITALTN | |
| Human | MRNRGQGLFRLRSRCFLHQSLPLGAGRRKGLDVAEPGPSRCRSDS | SEQ |
| AMPD2 | PAVAAVVPAMASYPSGSGKPKAKYPFKKRASLQASTAAPEARGG | ID |
| | LGAPPLQSARSLPGPAPCLKHFPLDLRTSMDGKCKEIAEELFTRSL | NO: |
| | AESELRSAPYEFPEESPIEQLEERRQRLERQISQDVKLEPDILLRAK | 890 |
| | QDFLKTDSDSDLQLYKEQGEGQGDRSLRERDVLEREFQRVTISGE | |
| | EKCGVPFTDLLDAAKSVVRALFIREKYMALSLQSFCPTTRRYLQQ | |
| | LAEKPLETRTYEQGPDTPVSADAPVHPPALEQHPYEHCEPSTMPG | |
| | DLGLGLRMVRGVVHVYTRREPDEHCSEVELPYPDLQEFVADVNV | |
| | LMALIINGPIKSFCYRRLQYLSSKFQMHVLLNEMKELAAQKKVPH | |
| | RDFYNIRKVDTHIHASSCMNQKHLLRFIKRAMKRHLEEIVHVEQG | |
| | REQTLREVFESMNLTAYDLSVDTLDVHADRNTFHRFDKFNAKYN | |
| | PIGESVLREIFIKTDNRVSGKYFAHIIKEVMSDLEESKYQNAELRLSI | |
| | YGRSRDEWDKLARWAVMHRVHSPNVRWLVQVPRLFDVYRTKG | |
| | QLANFQEMLENIFLPLFEATVHPASHPELHLFLEHVDGFDSVDDES | |
| | KPENHVFNLESPLPEAWVEEDNPPYAYYLYYTFANMAMLNHLRR | |
| | QRGFHTFVLRPHCGEAGPIHHLVSAFMLAENISHGLLLRKAPVLQ | |
| | YLYYLAQIGIAMSPLSNNSLFLSYHRNPLPEYLSRGLMVSLSTDDP | |
| | LQFHFTKEPLMEEYSIATQVWKLSSCDMCELARNSVLMSGFSHKV | |
| | KSHWLGPNYTKEGPEGNDIRRTNVPDIRVGYRYETLCQELALITQ | |
| | AVQSEMLETIPEEAGITMSPGPQ | |
| Mus | MASEARSGLGASPLQSARSLPGNAPCLKHFPLDLRTSMDGKCKEI | SEQ |
| musculus | AEELFSRSLAESELRSAPYEFPEESPIEQLEERRQRLERQISQDVKLE | ID |
| AMPD2 | PDILLRAKQDFLKTDSDSDLQLYKEQGEGQGDRGLWERDVVLER | NO: |
| | EFQRVIISGEEKCGVPFTDLLDAAKSVVRALFIREKYMALSLQSFC | 891 |
| | PTTRRYLQQLAEKPLETRTYEQSPDTPVSADAPVHPPALEQHPYE | |
| | HCEPSAMPGDLGLGLRMVRGVVHVYTRRDPDEHCPEVELPYPDL | |
| | QEFVADVNVLMALIINGPIKSFCYRRLQYLSSKFQMHVLLNEMKE | |
| | LAAQKKVPHRDFYNIRKVDTHIHASSCMNQKHLLRFIKRAMKRH | |
| | LEEIVHVEQGREQTLREVFESMNLTAYDLSVDTLDVHADRNTFHR | |
| | FDKFNAKYNPIGESVLREIFIKTDNKISGKYFAHIIKEVMADLEESK | |
| | YQNAELRLSIYGRSRDEWDKLARWAVNHKVHSPNVRWLVQVPR | |
| | LFDVYRTKGQLANFQEMLENIFLPLFEATVHPASHPELHLFLEHVD | |
| | GFDSVDDESKPENHVFNLESPLPEAWVEEDNPPYAYYLYYTFAN | |
| | MAMLNHLRRQRGFHTFVLRPHCGEAGPIHHLVSAFMLAENISHGL | |
| | LLRKAPVLQYLYYLAQIGIAMSPLSNNSLFLSYHRNPLPEYLSRGL | |
| | MVSLSTDDPLQFHFTKEPLMEEYSIATQVWKLSSCDMCELARNSV | |
| | LMSGFSHKVKSHWLGPNYTKEGPEGNDIRRTNVPDIRVGYRYETL | |
| | CQELALITQAVQSEMLETIPEEVGIVMSPGP | |
| Rattus | MASYPGPGKSKAKYPFKKRASLQASAAAPEARSGLGASPLQSARS | SEQ |
| norvegicus | LPGTAPCLKHFPLDLRTSMDGKCKEIAEELFSRSLAESELRSAPYEF | ID |
| AMPD2 | PEESPIEQLEERRQRLERQISQDVKLEPDILLRAKQDFLKTDSDSDL | NO: |
| | QLYKEQGEGQGDRGLWERDVVLEREFQRVIISGEEKCGVPFTDLL | 892 |
| | DAAKSVVRALFIREKYMALSLQSFCPTTRRYLQQLAEKPLETRTY | |
| | EQSPDTPVSADAPVHPPALEQHPYEHCEPSTMPGDLGLGLRMVRG | |
| | VVHVYTRRDPDEHCPEVELPYPDLQEFVADVNVLMALIINGPIKSF | |
| | CYRRLQYLSSKFQMHVLLNEMKELAAQKKVPHRDFYNIRKVDTH | |
| | IHASSCMNQKHLLRFIKRAMKRHLEEIVHVEQGREQTLREVFESM | |
| | NLTAYDLSVDTLDVHADRNTFHRFDKFNAKYNPIGESVLREIFIKT | |
| | DNKISGKYFAHIIKEVMSDLEESKYQNAELRLSIYGRSRDEWDKL | |
| | ARWAVNHRVHSPNVRWLVQVPRLFDVYRTKGQLANFQEMLENI | |
| | FLPLFEATVHPASHPELHLFLEHVDGFDSVDDESKPENHVFNLESP | |
| | LPEAWVEEDNPPYAYYLYYTFANMAMLNHLRRQRGFHTFVLRP | |
| | HCGEAGPIHHLVSAFMLAENISHGLLLRKAPVLQYLYYLAQIGIA | |
| | MSPLSNNSLFLSYHRNPLPEYLSRGLMVSLSTDDPLQFHFTKEPLM | |
| | EEYSIATQVWKLSSCDMCELARNSVLMSGFSHKVKSHWLGPNYT | |
| | KEGPEGNDIRRTNVPDIRVGYRYETLCQELALITQAVQSEMLETIP | |
| | EEVGIVMSPGP | |
| Plasmodium | MTILHEEINFLKKDELNINLKCLDKKERYKIWRRIPKCELHCHLDL | SEQ |
| gallinaceum | CFSLEFFLKCVRKYNLQPDLTDDEVVDYYLFKDKGKSLNEFIERS | ID |
| ADA | RRVTDIFINYDIIKDIAKNAVFNKYKEGVILIEFRYSPSYIAYKYNLC | NO: |
| | IDLIHKTIVEGINEAVEKLNHKIHVGLICIGETGISEESLRKAAEFCV | 893 |
| | KNKKDFVGFDHAGHERDLKPYKEIYDYVRENGIPLTIHAGEDLTL | |
| | PNLNTIYSAIEVLKAKRIGHGIRVIESEDLINLIKKNDILLEICPISNLL | |
| | LNNVKSMDTHPIKKLYDSGIKVSVNTDDPGMFLTEINDEYEELYL | |
| | NLNFNLEDFMKMNLWALEKSFVKSEIKDKLKKLYF | |
| Plasmodium | MNILQEPIDFLKKDELKNIDLSQMDKKERYKIWKRIPKCELHCHL | SEQ |
| knowlesi | DLCFSADFFLSCVRKYNLQPNLSDEEVLDYYLFAKGGKSLGEFVE | ID |
| ADA | KAIRVADIFQDYEMIEDLAKHAVFNKYKEGVVLMEFRYSPTFVAF | NO: |
| | KHNLDIELIHQAIVKGIKEVVELLDHKIDVTLLCIGDTGHRAADIK | 894 |
| | ASADFCLKHKADFVGFDHGGHEVDLKPYKEIFDYVKEGGMHLTV | |
| | HAGEDVTLPNLNTLYSAIQVLKVERIGHGIRVSESQELIDMVKENN | |
| | ILLEVCPISNVLLKNAKSFDTHPIRKLYDAGVKVSVSSDDPGMFLT | |
| | NINDDYEKLYTHLHFTLEDFMKMNEWALEKSFIGCDIKEKIKKLY | |
| | F | |
| Plasmodium | MEIPNEEIKFLKKEDIKNINLNGMNKKERYEIWKKIPKVELHCHLD | SEQ |
| berghei | LTFSGKFFLKWVRKYNLQPNMTDDQVLDHYLFTKEGKSLAEFIR | ID |
| ADA | KAISVSDIYRDYDILEDLAKWAVIEKYKEGVVLMEFRYSPTFVSSS | NO: |
| | HGLDIELIHKAFVKGIKNATEMLNNKIYVALICISDTGHSAASIKHS | 895 |
| | GDFAIKHKHDFVGFDHGGREIDLKDHKDVYHSVRNHGLHLTVHA | |
| | GEDATLPNLNTLYTAINILNVERIGHGIRVSESEELIELVKKNNILLE | |
| | VCPISNLLLNNVKSMDTHPIRKLFDAGVKVSVNSDDPGMFLTDIN | |
| | DNYEKLYIHLNFTLEEFMTMNNWALEKSFVNDDIKSKLKTMYF | |
| Plasmodium | MNILQEPIDFLKKEELKNIDLSQMSKKERYKIWKRIPKCELHCHLD | SEQ |
| vivax | LCFSADFFVSCIRKYNLQPNLSDEEVLDYYLFAKGGKSLGEFVEK | ID |
| ADA | AIKVADIFHDYEVIEDLAKHAVENKYKEGVVLMEFRYSPTFVAFK | NO: |
| | YNLDIELIHQAIVKGIKEVVELLDHKIHVALMCIGDTGHEAANIKA | 896 |
| | SADFCLKHKADFVGFDHGGHEVDLKEYKEIFDYVRESGVPLSVH | |
| | AGEDVTLPNLNTLYSAIQVLKVERIGHGIRVAESQELIDMVKEKNI | |
| | LLEVCPISNVLLKNAKSMDTHPIRQLYDAGVKVSVNSDDPGMFLT | |
| | NINDDYEELYTHLNFTLEDFMKMNEWALEKSFMDSNIKDKIKNL | |
| | YF | |
| Plasmodium | MNILQEPIDFLKKDEIKNIDLSQMSKKERYKIWKRIPKCELHCHLD | SEQ |
| cynomolgi | LCFSADFFLSCIRKYNLQPNLSDEEVLDYYLFAKGGKSLGEFVEKA | ID |
| ADA | IRVADIFHDYEVIEDLAKHAVFNKYKEGVVLMEFRYSPTFVAFKY | NO: |
| | KLDIELIHQAIVKGIKEVVELLDHKIHVALMCIGDTGHEAANIKAS | 897 |
| | ADFCLKHRADFVGFDHGGHEVDLKQYKEIFDYVRESGIPLSVHAG | |
| | EDVTLPNLNTLYSAIQVLKVERIGHGIRVSESQELIDMVKEKNILLE | |
| | VCPISNVLLKNAKSMDTHPIRQLYDAGVKVSVNSDDPGMFLTNIN | |
| | DDYEELYTHLNFTLEDFMKMNEWALEKSFMDSNIKDKVKNLYF | |
| Human | MAQTPAFDKPKVELHVHLDGSIKPETILYYGRRRGIALPANTAEG | SEQ |
| ADA | LLNVIGMDKPLTLPDFLAKFDYYMPAIAGCREAIKRIAYEFVEMK | ID |
| | AKEGVVYVEVRYSPHLLANSKVEPIPWNQAEGDLTPDEVVALVG | NO: |
| | QGLQEGERDFGVKARSILCCMRHQPNWSPKVVELCKKYQQQTV | 898 |
| | VAIDLAGDETIPGSSLLPGHVQAYQEAVKSGIHRTVHAGEVGSAE | |
| | VVKEAVDILKTERLGHGYHTLEDQALYNRLRQENMHFEICPWSS | |
| | YLTGAWKPDTEHAVIRLKNDQANYSLNTDDPLIFKSTLDTDYQM | |
| | TKRDMGFTEEEFKRLNINAAKSSFLPEDEKRELLDLLYKAYGMPP | |
| | SASAGQNL | |
| Mus | MAQTPAFNKPKVELHVHLDGAIKPETILYFGKKRGIALPADTVEE | SEQ |
| musculus | LRNIIGMDKPLSLPGFLAKFDYYMPVIAGCREAIKRIAYEFVEMKA | ID |
| ADA | KEGVVYVEVRYSPHLLANSKVDPMPWNQTEGDVTPDDVVDLVN | NO: |
| | QGLQEGEQAFGIKVRSILCCMRHQPSWSLEVLELCKKYNQKTVV | 899 |
| | AMDLAGDETIEGSSLFPGHVEAYEGAVKNGIHRTVHAGEVGSPEV | |
| | VREAVDILKTERVGHGYHTIEDEALYNRLLKENMHFEVCPWSSYL | |
| | TGAWDPKTTHAVVRFKNDKANYSLNTDDPLIFKSTLDTDYQMTK | |
| | KDMGFTEEEFKRLNINAAKSSFLPEEEKKELLERLYREYQ | |
| Bos taurus | MAQTPAFNKPKVELHVHLDGAIKPETILYYGRKRGIALPADTPEEL | SEQ |
| ADA | QNIIGMDKPLSLPEFLAKFDYYMPAIAGCREAVKRIAYEFVEMKA | ID |
| | KDGVVYVEVRYSPHLLANSKVEPIPWNQAEGDLTPDEVVSLVNQ | NO: |
| | GLQEGERDFGVKVRSILCCMRHQPSWSSEVVELCKKYREQTVVAI | 900 |
| | DLAGDETIEGSSLFPGHVKAYAEAVKSGVHRTVHAGEVGSANVV | |
| | KEAVDTLKTERLGHGYHTLEDATLYNRLRQENMHFEVCPWSSYL | |
| | TGAWKPDTEHPVVRFKNDQVNYSLNTDDPLIFKSTLDTDYQMTK | |
| | NEMGFTEEEFKRLNINAAKSSFLPEDEKKELLDLLYKAYGMPSPA | |
| | SAEQCL | |
| Gallus | MERGVRVFGEPKVELHIHLDGAIRPETILHFGKKRGVPLPGSTVDE | SEQ |
| gallus | LMKHVSYQTPLSLKLFLEKFNHYMPAIAGDREAVRRIAYELVETK | ID |
| ADA | AKEGVVYVEVRYSPHLLANCRVEPIPWGQAEGDLTPEEVVNLVN | NO: |
| | QGLQDGERNFRIKARSILCCMRHMPSWSPEVVELCKKYQNNSVV | 901 |
| | AIDLAGDELLMASSDHKAAYEEAERCGIHRTVHAGEAGPATMIKE | |
| | AVYLLKAERIGHGYHVLEDPELYRELLRTRMHFEVCPWSSYLTG | |
| | ACLPDFRKHPVVQFKKDQANYSINTDDPLIFNSNIDKDYGIVKEY | |
| | MDFTEEDFKRVNINAAQSSFLPEKEKQELLNTLYEAYGMVPATS | |
| Danio | MNGKPAFDKPKVELHVHLDGAIRLKTVLDVAKRRGISLPVSMEEE | SEQ |
| rerio ADA | LKELCTVNEPATLTEFLGKFSHFMHVIAGDREAIKRIAYEFVETKA | ID |
| | KEGVIYVEARYSPHFLANKGVEPLPWDQKPGDITPDDVVDLVNQ | NO: |
| | GFKEGEQAFKTKARSILCCMRHMPNWSMEVVELCKKFHKDGVV | 902 |
| | AIDLAGDESMNCESYPGHKKAFEEAVRSNVHRTVHAGEVGPASV | |
| | VREAVEVLKAERIGHGYHTLEDQNLYKQLLHQNMHFEMCPVSSR | |
| | LTGACEPDFTKHPLITFKKDKANYSLNTDDPTIFNSTLNSDYEVVQ | |
| | KYMDFTEEEFKRLNINAAKSCFLPEKEKEKLLNQLYEAYGMRKST | |
| | SF | |
| Xenopus | MESKAFNKPKVELHVHLDGSIKPETIIHFAKKRQIKLPADTVEGLL | SEQ |
| laevis | EHVSYKEPLSLTEFLQKFNHYMPAIAGDREAIKRIAYEFVEMKAK | ID |
| ADA | EGVIYVEVRYSPHFLANSKVDPIPWGQKEGDITPDEVVDLVNQGL | NO: |
| | RKGEKTFNIKARSILCCMRHMPNWSSEVIELCKKYQNDTVVAIDL | 903 |
| | AGDESLNCESYPGHRKAYEEAVKCGIHRTVHAGEVGPPSVVKEA | |
| | VEVLKAERIGHGYHTTEDPNLYKELLENNMHFEVCPWSSYLTSAC | |
| | HPDFTKHPATQFRKDKANFSLNTDDPLIFGSTLDVDYSIAVQHMG | |
| | FTEDEFKRVNINAAKSSFLPDNEKKELLYKLYEAYGMILSTGL | |
| Plasmodium | MNCKNMDTSYEIINYLTKDELDIDLSCMDKKERYKIWKRLPKCEL | SEQ |
| falciparum | HCHLDVCFSVDFFLNVIRKYNIQPNMSDEEIIDYYLFSKPGKSLDEF | ID |
| ADA | VEKALRLTDIYIDYTVVEDLAKHAVENKYKEGVVLMEFRYSPSF | NO: |
| | MSFKHNLDKDLIHEAIVKGLNEAVALLEYKIQVGLLCTGDGGLSH | 904 |
| | ERMKEAAEFCIKHKKDFVGYDHAGHEVDLKPFKDIFDNIREEGISL | |
| | SVHAGEDVSIPNLNSLYTAINLLHVKRIGHGIRVSESQELIDLVKEK | |
| | DILLEVCPISNVLLNNVKSMDTHPIRMLYDAGVKVSVNSDDPGMF | |
| | LTNITDNYEELYTHLNFTLADFMKMNLWAVQKSFVDPDIKNKIIS | |
| | KYF | |
| Drosophila | MEQFLKGLPKVELHAHLNGSLGIKSLCDLGERLYGTSCKDFLKLC | SEQ |
| melancgaster | AHFSRFEKDMDACFEKFAFVHELTSTREGLRFATELAIRDFAEDN | ID |
| ADA | VQYVEMRTTPKANENYSRRDYLQIVIDAIKAASETYPEITVKLLPSI | NO: |
| | NRAEPVDVAEETVSLAVELARAHPNLILGIDLSGNPGKGRFSDFAP | 905 |
| | ILAQARDKGLKLAIHCAEIENPSEVKEMLHFGMSRCGHGTFLTPE | |
| | DIGQLKQRNIAIECCLTSNVKSGTVPSLEEHHLKRIMEADAPKVIC | |
| | TDDSGVFDTTLTKEFLIAAETFGLTREQCIDLTLEAVHHSFASEQE | |
| | QIQMADRVGNYADILVK | |
| Xenopus | MESKAFNKPKVELHVHLDGSIKPETIIHFAKKRQIKLPADTVEGLL | SEQ |
| tropicalis | EHVSYKEPLSLTEFLSKFNHYMPAIAGDREAIKRIAYEFVEMKAKE | ID |
| ADA | GVIYVEVRYSPHFLANSKVEPIPWGQKEGDITPDEVVDLVNQGLR | NO: |
| | KGEKAFNIKARSILCCMRHMPSWSTEVVELCKKYQNDTVVAIDL | 906 |
| | AGDESLNCESYPGHRKAYEEAVKCGIHRTVHAGEVGPSSVVKEA | |
| | VEVLKAERIGHGYHTTEDPNLYKELLEKNMHFEVCPWSSYLTGA | |
| | CHPDFTKHPATQFRKDKANYSLNTDDPLIFGSTLDVDYSIAAKHM | |
| | GFTEEEFKRVNINAAKSSFLPESEKKELLYKLYEAYGMILSTGL | |
| Rattus | MAQTPAFNKPKVELHVHLDGAIKPETILYYGKKRGIDLPADTVEG | SEQ |
| norvegicus | LRNIIGMDKPLSLPDFLAKFDYYMPAIAGCREAIKRIAYEFVEMKA | ID |
| ADA | KEGVVYVEVRYSPHLLANSKVDPIPWNQAEGDLTPDEVVDLVNQ | NO: |
| | GLQEGEQAFGIKVRSILCCMRHQPSWSPEVLELCKKYHQKTVVA | 907 |
| | MDLAGDETIEGSSLFPGHVEAYEGAVKDGIHRTVHAGEVGSAEV | |
| | VREAVDILKTERVGHGYHTIEDEALYNRLLKENMHFEVCPWSSYL | |
| | TGAWNPKTTHAVVRFKDDQANYSLNSDDPLIFKSTVDTDYQMVK | |
| | KDMGFTEEEFKRLNINAAKSSFLPEDEKKELLERLYKEYQ | |
| Mus | MATAGGSRRAPVPGPRLGLPLAAHLPASLGGEGAKDSVGGEKTS | SEQ |
| musculus | GNNDWFQSSRVPSFAQMLKKNLPVQPSAQTVTLPTGYSSESCSLS | ID |
| ADAD1 | NMASKVTQVTGNFPEPLLSKGLSSISNPVLPPKKLPKEFIMKYKRG | NO |
| | EINPVSALHQFAQMQRVQLDLKETVTTGNVMGPYFAFCAVVDGI | 908 |
| | QYKTGLGQNKKESRSNAAKLALDELLQLDEPEPRVLEPAGPPPIPA | |
| | EPVVTPEAAYVSKVQYEGRQVQYAKISQLVKETFGQLISNHSQYL | |
| | KCSSSLAAFIIERAGHHEVVAIGTGEYNYSQCIKPNGRVLHDTHAV | |
| | VTARRSLLRYFYRQLLLFYSKNPAMMEKSIFCTEPASNLLTLKQNI | |
| | NLYLYMNQLPKGSAQIKSQLRLNPHSISAFEANEELSLHVAVEGKI | |
| | YLTVYCSADGVNRVNSMSSSDKLTRWEVLGVQGALLSHFIQPVYI | |
| | SSILVGDGNCNDTRGLEIAINQRVDDALTSKLPMFYLVNRPHISLV | |
| | PTAYPLQINLDHKSLSLNWAQGDNSLEIVDGLNGKITESSPFKSGL | |
| | SMASRLCKAAMLSRFNLLAKEAKTDDLLEARTYHAAKCMSGPY | |
| | QEAKALLKAYLQQHGYGSWIVKSPCIEQFSM | |
| Rattus | MATAGGSRRAPVPGPRLGLPLAAHLPASLGGEGAKDSLGGEKTS | SEQ |
| norvegicus | GNNDWFQSSRVPSFAQMLKKNLPVQSSAQTVTLPTGYSSESCSLS | ID |
| ADAD1 | NMASKVTQVTGNFPEPLLSKGLSSISNPVLPPKKIPKEFIMKYRRG | NO: |
| | EINPVSALHQFAQMQRVQLDLKETVTTGNVMGPYFAFCAVVDGI | 909 |
| | QYKTGLGQNKKESRSNAAKLALDELLQLDEPEPRALEPAGPPPIPA | |
| | EPIVTPEAAYISKVQYEGRQVQYAKISQLVKETFSQLISSHSQYLKC | |
| | SSSLAAFIIERGGHHEVVAIGTGEYNYSQCIKPNGRVLHDTHAVVT | |
| | ARRSLLRYFYRQLLLFYSKNPAMMEKSIFCTEPASNLLTLKQNINL | |
| | YLYMNQLPKGSAQIKSQLRLNPHSISAFEANEELSLHVAVEGKIYL | |
| | TVYCSADGVNRVNSMSSSDKLTRWEVLGVQGALLSHFIQPVYISS | |
| | ILVGDGNCNDTRGLEIAINQRVDDALTSKLPMFYLVNRPHISLVPT | |
| | AYPLQINLDHKSLSLNWAQGDNSLEIVDGLSGKITESSPFKSGLSM | |
| | ASRLCKAAMLSRFNLLAKEAKTDDLLEARTYHAAKCLSGPYQEA | |
| | KALLKAYLQQHGYGSWIVKSPCIEQFSM | |
| Xenopus | MASNRNWSQHSSVPSFAQMLKKNLPDPGTSPAVNQTSTLSTCCL | SEQ |
| laevis | YNQPDCGTARVTRITGNFPEPFLSKMIVPPSLSSLTPRKVTKEFMV | ID |
| ADAD1 | KYRRGDLNPISALYQFAQMQRMEIELKETVTTGNVFGAYFAFCA | NO: |
| | VVDGLEYKTGMGQNKKEAKANAAKLALDELLLHEDPALIDSENQ | 910 |
| | SLNVIENPPPLPMNPRGTTETSTISRTRTDKRTFIHEKISSIIKETFTN | |
| | LVSKYPEYENCGSSLAAFVIEKGGQHWEVVAIGTGEFNYGQSLQS | |
| | DGRVLHDSHAMVVARRSLLRYFYRQLLLLYSGNNGMMDKSIFCT | |
| | EPATNLLALKPNLNIFLYMNQLPKGAAQTNPQLCLSPHSLSAHEA | |
| | NDKLSLHVSVEGKNIPASYYSGEIVHNLYSMSSTDKLTRWEVLGV | |
| | QGALLSIFIQPVYINSIIIGNAACSDTRGLEIAVKQRIDDALTSRLPM | |
| | FYLVNRPYMSIVSSTHLTNNDTANKTLSLNWSQGDACVEVVDAA | |
| | IGRTVEGSPFKSGSCLASRLCKAAMLCRFNLVVKESKRNAIPSGLS | |
| | YHEAKRLAGPYQEAKCLLNSYFKQQGFGSWIAKPPIIGEFTM | |
| Human | MASNNHWFQSSQVPSFAQMLKKNLPVQPATKTITTPTGWSSESY | SEQ |
| ADAD1 | GLSKMASKVTQVTGNFPEPLLSKNLSSISNPVLPPKKIPKEFIMKYK | ID |
| | RGEINPVSALHQFAQMQRVQLDLKETVTTGNVMGPYFAFCAVVD | NO: |
| | GIQYKTGLGQNKKESRSNAAKLALDELLQLDEPEPRILETSGPPPF | 911 |
| | PAEPVVLSELAYVSKVHYEGRHIQYAKISQIVKERFNQLISNRSEY | |
| | LKYSSSLAAFIIERAGQHEVVAIGTGEYNYSQDIKPDGRVLHDTHA | |
| | VVTARRSLLRYFYRQLLLFYSKNPAMMEKSIFCTEPTSNLLTLKQ | |
| | NINICLYMNQLPKGSAQIKSQLRLNPHSISAFEANEELCLHVAVEG | |
| | KIYLTVYCPKDGVNRISSMSSSDKLTRWEVLGVQGALLSHFIQPV | |
| | YISSILIGDGNCSDTRGLEIAIKQRVDDALTSKLPMFYLVNRPHISL | |
| | VPSAYPLQMNLEYKFLSLNWAQGDVSLEIVDGLSGKITESSPFKSG | |
| | MSMASRLCKAAMLSRFNLLAKEAKKELLEAGTYHAAKCMSASY | |
| | QEAKCKLKSYLQQHGYGSWIVKSPCIEQFNM | |
| Bos taurus | MMEAEEQPWKTTFYSKLPKVELHAHLNGSISSNTIRKLIAKKPDL | SEQ |
| ADAL | KIHDQMTMIDKGEKRTLEECLQMFQIIHLLTTTPEDVLMVTKDVI | ID |
| | KEFADDGVKYLELRSTPRGEDATGMTKKTYVESILEGIKQSKEEN | NO: |
| | VDIDVRYLISIDRRGGSSAAKEAVKLAEEFFLSAEDTVLGLDLSGD | 912 |
| | PSAGQAKDFLEPLLEAKKSGLKLALHLSEIPNQKTETQVLLNLFPD | |
| | RIGHGTFLSSSEEGSPDLVDFVRQHQIPLELCLTSNVKSQTVPAYD | |
| | QHHFGFWYSVAHPAVICTDDKGVFATRLSQEYQLVAETFHLTQS | |
| | QVWDLSYESISYIFASDSTKADLRKKWSHLKPHF | |
| Danio | MDTEADLFYRQLPKVELHAHLNGSVSFETMEKLIKRKPHLNIEHS | SEQ |
| rerio | MTAIRRGQRRTLDECFQVFKVIHQLVDSEEDILMVAKSVIQEFAA | ID |
| ADAL | DGVKYLELRSTPREVTETGLSKQRYIETVLEAIRQCKQEGVDIDVR | NO: |
| | FLVAVDRRHGPEVAMQTVKLAEDFLLSSDGTVVGLDLSGDPTVG | 913 |
| | HGKDLLAALQKAKNCGLKLALHLSEVPSQIDETELLLNLPPDRIG | |
| | HGTFLHPDVGGSDSLVDKVCKQNIPIEICLTSNVKGQTVPSYDKH | |
| | HFKYWYNRGHPCVLCTDDKGVFCTDLSQEYQLAASTFGLTKEAV | |
| | WILSQQAIGYTFAPEPIKQRLEKTWAELKQQILQ | |
| Xenopus | MAGEGALQFYRDLPKVELHAHLNGSISTATMKKLMARKPHLDIQ | SEQ |
| laevis | HGMTMIDKGQKRTLEECFQMFKIIHQITDTAEDILLVTKDVIKEFA | ID |
| ADAL | ADGVKYLELRSTPRDTPAGLTKQAYVETVLEGIKQCKEEGVDIDV | NO: |
| | RFLLAIDRRGGPTAAKETVKLAEDFFCSSNELVLGLDLSGDPTVG | 914 |
| | HGRDFMEPLNKARQSGLKLALHLSEIPSQTEETELLLGLPPDRIGH | |
| | GTFLTTSAHIVEIVKKQHIPLELCITSNIKGQTVSSYNEHHFGFWYN | |
| | LHHPFVLCTDDKGVFATDLSVEYEIAAKTFNLTPHHVWDLSYQAI | |
| | DYTFASADVKANLKEKWLLLKPDVFRHAL | |
| Human | MIEAEEQQPCKTDFYSELPKVELHAHLNGSISSHTMKKLIAQKPDL | SEQ |
| ADAL | KIHDQMTVIDKGKKRTLEECFQMFQTIHQLTSSPEDILMVTKDVIK | ID |
| | EFADDGVKYLELRSTPRRENATGMTKKTYVESILEGIKQSKQENL | NO: |
| | DIDVRYLIAVDRRGGPLVAKETVKLAEEFFLSTEGTVLGLDLSGDP | 915 |
| | TVGQAKDFLEPLLEAKKAGLKLALHLSEIPNQKKETQILLDLLPDR | |
| | IGHGTFLNSGEGGSLDLVDFVRQHRIPLELCLTSNVKSQTVPSYDQ | |
| | HHFGFWYSIAHPSVICTDDKGVFATHLSQEYQLAAETFNLTQSQV | |
| | WDLSYESINYIFASDSTRSELRKKWNHLKPRVLHI | |
| Caenorhabditis | MPNNSKHKKKQQRRQQEAQKKSRAKQIETDKKNDEFLDTELDEV | SEQ |
| elegans | SPLVIDDDMTEFKNMPKVELHAHLSGSLSPETIKLIMESDETRAEEI | ID |
| ADAL | MKKYKLEKPENMTGVFDCFPVIHAILRKPEAIRIAIRQTIKEFEEDN | NO: |
| | CVYLELRTSPKETDFMTYEDYLQVCIESFEAAKHEFPRIKTFLIVSL | 916 |
| | DRRMPFETAAHILGLIGEAQQRTNVIVGVELSGDPHLDGRRLLKLF | |
| | VAARRFHGLGITIHLAEVLQNMADVEDYLNLRPDRIGHGTFLHTD | |
| | PYTEYLTNKYKIPLEICLSSNVYSKTTTNYRNSHFNYWRKRGVPV | |
| | FICTDDKGVIPGATLTEEYYKAAITFDLSTEELIGINQDALLNSFAY | |
| | KYNVTDLTETFRKINNNVLD | |
| Mus | MEAGQQWPGKTDFYLQLPKVELHAHLNGSISSSTMKKLIAKKPH | SEQ |
| musculus | LNVHGHMTMIDKGKKRTLQECFQMFQVIHQLTTSAEDILMVTKD | ID |
| ADAL | VIKEFADDGVKYLELRSTPREENATGMTRKTYVESVLEGIKQCKQ | NO: |
| | ENLDIDVRYLMAIDRRGGPTIARETVELAKEFFLSTENTVLGLDLS | 917 |
| | GDPTIGQANDFLEPLLEAKKAGLKLALHLAEIPNREKENQMLLSL | |
| | LPDRIGHGTFLSASEAGALDQVDFVRQHQIPLELCLTSNIKSQTVPS | |
| | YDQHHFGFWYSIAHPSVICTDDKGVFATYLSQEYQLAAETFNLTP | |
| | FQVWDLSYESINYIFACDNTRSELRKRWTHLKQKVLNCNEVNYF | |
| Human | MASASQGADDDGSRRKPRLAASLQISPQPRPWRPLPAQAQSAWG | SEQ |
| ADAD2 | PAPAPATYRAEGGWPQVSVLRDSGPGAGAGVGELGAARAWENL | ID |
| | GEQMGKAPRVPVPPAGLSLPLKDPPASQAVSLLTEYAASLGIFLLF | NO: |
| | REDQPPGPCFPFSVSAELDGVVCPAGTANSKTEAKQQAALSALCY | 918 |
| | IRSQLENPESPQTSSRPPLAPLSVENILTHEQRCAALVSAGFDLLLD | |
| | ERSPYWACKGTVAGVILEREIPRARGHVKEIYKLVALGTGSSCCA | |
| | GWLEFSGQQLHDCHGLVIARRALLRFLFRQLLLATQGGPKGKEQS | |
| | VLAPQPGPGPPFTLKPRVFLHLYISNTPKGAARDIYLPPTSEGGLPH | |
| | SPPMRLQAHVLGQLKPVCYVAPSLCDTHVGCLSASDKLARWAVL | |
| | GLGGALLAHLVSPLYSTSLILADSCHDPPTLSRAIHTRPCLDSVLGP | |
| | CLPPPYVRTALHLFAGPPVAPSEPTPDTCRGLSLNWSLGDPGIEVV | |
| | DVATGRVKANAALGPPSRLCKASFLRAFHQAARAVGKPYLLALK | |
| | TYEAAKAGPYQEARRQLSLLLDQQGLGAWPSKPLVGKFRN | |
| Mus | MASVDEGGRRRPRLAASLQISPGPWKPSGGQEPTEAGDAAPRTAE | SEQ |
| musculus | HGVAGAQEAHREACKALGGSVLSPGPAGDFPGALHGLSMLPKDP | ID |
| ADAD2 | PPAQAVALLTQCMANLGVSLTFLEDQTAGPGSSFSVCADLDGLVC | NO: |
| | PAGTGSSKLEAKQQAALSALQYIQKQLERPEPLVTPRQPLLTSLSIE | 919 |
| | TILTHEQRCAAVVSAGLDRLLSESSPYQACKGTVAAVILEREVQG | |
| | SIGHSKETYELVALGTGSSSCAGWLEFSGRRLHDCHGLVIARRAL | |
| | LRFFFRQLLLVTQGGPKGQERSVLTPQPGPGPPFALKPGVFLHLYV | |
| | SNTPKGAAHDIYLPLASEDSVLHSPAFRLQAHVCGQLKPVSYVAP | |
| | ALRDTHVGCLSASDKLARWAILGLGGGLLAHFLPPLYATSLVLAD | |
| | PCHDPSTLNRVIHSRPRLDSVLGSCLPCPYVRTTLHLFAGPLVAPS | |
| | DPGPSTCHSLSLNWSLGDPDIEVVDVATGRVKTDSSVGPPSRLCK | |
| | AAFLSAFRQVARALEKPQLLSLQTYEAAKAVPYREARQQLSLLLD | |
| | QQGLGAWPSKPLVGKFRH | |
| Macaca | MASASQGADDGSRRKPRLAASLQISPEPRPWRPLPPQAQGAWEPA | SEQ |
| fascicularis | PAMDHAEGGQPQVSVLRDSGPGAGAGVGELGAAQAWENLGEQ | ID |
| ADAD2 | MGRTPRVPVPPAGLSLPLKDPPASQAVSLLTEYAASLGIILLFRED | NO: |
| | QQPGPCFPFSVSAELDGVVCPAGTANSKTEAKQQAALSALCYIRS | 920 |
| | QLESPESPQTSSRPPPPPLSVDSILTHGQRCAALVSAGFDLLLDERS | |
| | PYWACKGTVAGVILEREIPGARGHVKEIYKLVALGTGSSCCAGW | |
| | LEFSGQQLHDCHGLVIARRALLRFLFRQLLLATQGGAKGKEQSVL | |
| | APQPGPGPPFTLKPRVFLHLYISNTPKGAARDIYLPPTSEGGLPHSP | |
| | PMRLQAHVLGQLKPVCYVAPSLCDTHVGCLSASDKLARWAVLG | |
| | LGGALLAHLVSPLYSTSLILADSCHDPPTLSRAIHTRPCLDSVLGPC | |
| | LPPPYVRTALHLFSGPPVAPSEPTPDTCHGLSLNWSLGDPGIEVVD | |
| | VATGRVKANAALGPPSRLCKASFLRAFHQVARAVGKPYLLALKT | |
| | YEAAKAGPYQEARRQLSLLLDQQGLGAWPSKPLVGKFRN | |

1. A gene editing system comprising a guide RNA (mgRNA) and a helper guide RNA (hgRNA), or at least one DNA polynucleotide encoding the mgRNA and/or the hgRNA, wherein the mgRNA comprises a mgRNA spacer targeting a T-cell receptor α constant (TRAC) gene and the hgRNA comprises a hgRNA spacer, wherein the nucleic acid sequence of the mgRNA spacer comprises a sequence selected from SEQ ID NOs: 1-5;
or
wherein the mgRNA comprises a mgRNA spacer targeting a CD52 gene and the hgRNA comprises a hgRNA spacer, wherein the nucleic acid sequence of the mgRNA spacer comprises a sequence selected from SEQ ID NOs: 6-8;
or
wherein the mgRNA comprises a mgRNA spacer targeting a b2-microglobulin (B2M) gene and the hgRNA comprises a hgRNA spacer, wherein the nucleic acid sequence of the mgRNA spacer comprises a sequence selected from SEQ ID NOs: 9-19;
or
wherein the mgRNA comprises a mgRNA spacer targeting a programmed cell death protein 1 (PDCD1) gene and the hgRNA comprises a hgRNA spacer, wherein the nucleic acid sequence of the mgRNA spacer comprises a sequence selected from SEQ ID NOs: 20-38;
or
wherein the mgRNA comprises a mgRNA spacer targeting a cytotoxic T-lymphocyte associated protein 4 (CTLA4) gene and the hgRNA comprises a hgRNA spacer, wherein the nucleic acid sequence of the mgRNA spacer comprises a sequence selected from SEQ ID NOs: 247-256;
or
wherein the mgRNA comprises a mgRNA spacer targeting a T cell immunoreceptor with Ig and ITIM domains (TIGIT) gene and the hgRNA comprises a hgRNA spacer, wherein the nucleic acid sequence of the mgRNA spacer comprises a sequence selected from SEQ ID NOs: 278-294;
or
wherein the mgRNA comprises a mgRNA spacer targeting a hepatitis A virus cellular receptor 2 (HAVCR2/TIM3) gene and the hgRNA comprises a hgRNA spacer, wherein the nucleic acid sequence of the mgRNA spacer comprises a sequence selected from SEQ ID NOs: 323-337;
or
wherein the mgRNA comprises a mgRNA spacer targeting a lymphocyte activating 3 (LAG3) gene and the hgRNA comprises a hgRNA spacer, wherein the nucleic acid sequence of the mgRNA spacer comprises a sequence selected from SEQ ID NOs: 364-396;
or
wherein the mgRNA comprises a mgRNA spacer targeting a cytokine inducible SH2 containing protein (CISH) gene and the hgRNA comprises a hgRNA spacer, wherein the nucleic acid sequence of the mgRNA spacer comprises a sequence selected from SEQ ID NOs: 472-482;
or
wherein the mgRNA comprises a mgRNA spacer targeting a transforming growth factor beta receptor 2 (TGFBR2) gene and the hgRNA comprises a hgRNA spacer, wherein the nucleic acid sequence of the mgRNA spacer comprises a sequence selected from SEQ ID NOs: 504-510;
or
wherein the mgRNA comprises a mgRNA spacer targeting a Fas cell surface death receptor (FAS) gene and the hgRNA comprises a hgRNA spacer, wherein the nucleic acid sequence of the mgRNA spacer comprises a sequence selected from SEQ ID NOs: 530-541;
or
wherein the mgRNA comprises a mgRNA spacer targeting a CD7 gene and the hgRNA comprises a hgRNA spacer, wherein the nucleic acid sequence of the mgRNA spacer comprises a sequence selected from SEQ ID NOs: 565-575;
or
wherein the mgRNA comprises a mgRNA spacer targeting a Cb1 proto-oncogene B (CBLB) gene and the hgRNA comprises a hgRNA spacer, wherein the nucleic acid sequence of the mgRNA spacer comprises a sequence selected from SEQ ID NOs: 609-618;
or
wherein the mgRNA comprises a mgRNA spacer targeting a killer cell lectin like receptor C1 (KLRC1) gene and the hgRNA comprises a hgRNA spacer, wherein the nucleic acid sequence of the mgRNA spacer comprises a sequence selected from SEQ ID NOs: 637-641;
or
wherein the mgRNA comprises a mgRNA spacer targeting a CD38 gene and the hgRNA comprises a hgRNA spacer, wherein the nucleic acid sequence of the mgRNA spacer comprises a sequence selected from SEQ ID NOs: 651-659.

2-117. (canceled)

118. The gene editing system of claim 1, wherein the nucleic acid sequences of the mgRNA spacer and the hgRNA spacer comprise respectively:

| mgRNA | hgRNA |
|---|---|
| SEQ ID NO: 01 | SEQ ID NO: 39 |
| SEQ ID NO: 01 | SEQ ID NO: 40 |
| SEQ ID NO: 01 | SEQ ID NO: 41 |
| SEQ ID NO: 02 | SEQ ID NO: 39 |
| SEQ ID NO: 02 | SEQ ID NO: 40 |
| SEQ ID NO: 02 | SEQ ID NO: 41 |
| SEQ ID NO: 03 | SEQ ID NO: 42 |
| SEQ ID NO: 03 | SEQ ID NO: 43 |
| SEQ ID NO: 03 | SEQ ID NO: 44 |
| SEQ ID NO: 04 | SEQ ID NO: 45 |
| SEQ ID NO: 04 | SEQ ID NO: 46 |
| SEQ ID NO: 04 | SEQ ID NO: 47 |
| SEQ ID NO: 05 | SEQ ID NO: 48 |
| SEQ ID NO: 05 | SEQ ID NO: 49; or |
| SEQ ID NO: 05 | SEQ ID NO: 50 |
| SEQ ID NO: 679 | SEQ ID NO: 680 |
| SEQ ID NO: 679 | SEQ ID NO: 681 |
| SEQ ID NO: 682 | SEQ ID NO: 684 |
| SEQ ID NO: 682 | SEQ ID NO: 685; or |
| SEQ ID NO: 683 | SEQ ID NO: 685 |
| SEQ ID NO: 06 | SEQ ID NO: 51 |
| SEQ ID NO: 06 | SEQ ID NO: 52 |
| SEQ ID NO: 06 | SEQ ID NO: 53 |
| SEQ ID NO: 07 | SEQ ID NO: 51 |
| SEQ ID NO: 07 | SEQ ID NO: 52 |
| SEQ ID NO: 07 | SEQ ID NO: 53 |
| SEQ ID NO: 08 | SEQ ID NO: 54 |
| SEQ ID NO: 08 | SEQ ID NO: 55; or |
| SEQ ID NO: 08 | SEQ ID NO: 56 |
| SEQ ID NO: 686 | SEQ ID NO: 688 |
| SEQ ID NO: 687 | SEQ ID NO: 688; or |
| SEQ ID NO: 689 | SEQ ID NO: 690 |
| SEQ ID NO: 09 | SEQ ID NO: 57 |
| SEQ ID NO: 09 | SEQ ID NO: 58 |
| SEQ ID NO: 09 | SEQ ID NO: 59 |
| SEQ ID NO: 09 | SEQ ID NO: 60 |
| SEQ ID NO: 09 | SEQ ID NO: 61 |
| SEQ ID NO: 10 | SEQ ID NO: 62 |
| SEQ ID NO: 11 | SEQ ID NO: 63 |
| SEQ ID NO: 11 | SEQ ID NO: 64 |
| SEQ ID NO: 11 | SEQ ID NO: 65 |
| SEQ ID NO: 11 | SEQ ID NO: 66 |
| SEQ ID NO: 12 | SEQ ID NO: 67 |
| SEQ ID NO: 12 | SEQ ID NO: 68 |
| SEQ ID NO: 12 | SEQ ID NO: 69 |
| SEQ ID NO: 12 | SEQ ID NO: 70 |
| SEQ ID NO: 12 | SEQ ID NO: 71 |
| SEQ ID NO: 13 | SEQ ID NO: 67 |
| SEQ ID NO: 13 | SEQ ID NO: 68 |
| SEQ ID NO: 13 | SEQ ID NO: 69 |
| SEQ ID NO: 13 | SEQ ID NO: 70 |
| SEQ ID NO: 13 | SEQ ID NO: 71 |
| SEQ ID NO: 14 | SEQ ID NO: 67 |
| SEQ ID NO: 14 | SEQ ID NO: 68 |
| SEQ ID NO: 14 | SEQ ID NO: 69 |
| SEQ ID NO: 14 | SEQ ID NO: 70 |
| SEQ ID NO: 14 | SEQ ID NO: 71 |
| SEQ ID NO: 15 | SEQ ID NO: 72 |
| SEQ ID NO: 15 | SEQ ID NO: 73 |
| SEQ ID NO: 15 | SEQ ID NO: 74 |
| SEQ ID NO: 15 | SEQ ID NO: 75 |
| SEQ ID NO: 15 | SEQ ID NO: 76 |
| SEQ ID NO: 16 | SEQ ID NO: 72 |
| SEQ ID NO: 16 | SEQ ID NO: 73 |
| SEQ ID NO: 16 | SEQ ID NO: 74 |
| SEQ ID NO: 16 | SEQ ID NO: 75 |
| SEQ ID NO: 16 | SEQ ID NO: 76 |
| SEQ ID NO: 17 | SEQ ID NO: 77 |
| SEQ ID NO: 17 | SEQ ID NO: 78 |
| SEQ ID NO: 17 | SEQ ID NO: 79 |
| SEQ ID NO: 17 | SEQ ID NO: 80 |
| SEQ ID NO: 18 | SEQ ID NO: 77 |
| SEQ ID NO: 18 | SEQ ID NO: 78 |
| SEQ ID NO: 18 | SEQ ID NO: 79 |
| SEQ ID NO: 18 | SEQ ID NO: 80 |
| SEQ ID NO: 19 | SEQ ID NO: 81; or |
| SEQ ID NO: 19 | SEQ ID NO: 82 |
| SEQ ID NO: 697 | SEQ ID NO: 698; or |
| SEQ ID NO: 699 | SEQ ID NO: 700 |
| SEQ ID NO: 20 | SEQ ID NO: 83 |
| SEQ ID NO: 20 | SEQ ID NO: 84 |
| SEQ ID NO: 20 | SEQ ID NO: 85 |
| SEQ ID NO: 20 | SEQ ID NO: 86 |
| SEQ ID NO: 21 | SEQ ID NO: 87 |
| SEQ ID NO: 22 | SEQ ID NO: 87 |
| SEQ ID NO: 23 | SEQ ID NO: 88 |
| SEQ ID NO: 23 | SEQ ID NO: 89 |
| SEQ ID NO: 23 | SEQ ID NO: 90 |
| SEQ ID NO: 24 | SEQ ID NO: 91 |
| SEQ ID NO: 24 | SEQ ID NO: 92 |
| SEQ ID NO: 24 | SEQ ID NO: 93 |
| SEQ ID NO: 25 | SEQ ID NO: 94 |
| SEQ ID NO: 25 | SEQ ID NO: 95 |
| SEQ ID NO: 25 | SEQ ID NO: 96 |
| SEQ ID NO: 26 | SEQ ID NO: 97 |
| SEQ ID NO: 26 | SEQ ID NO: 98 |
| SEQ ID NO: 26 | SEQ ID NO: 96 |
| SEQ ID NO: 27 | SEQ ID NO: 99 |
| SEQ ID NO: 27 | SEQ ID NO: 100 |
| SEQ ID NO: 28 | SEQ ID NO: 99 |
| SEQ ID NO: 28 | SEQ ID NO: 101 |
| SEQ ID NO: 28 | SEQ ID NO: 102 |
| SEQ ID NO: 29 | SEQ ID NO: 103 |
| SEQ ID NO: 29 | SEQ ID NO: 104 |
| SEQ ID NO: 29 | SEQ ID NO: 105 |
| SEQ ID NO: 30 | SEQ ID NO: 106 |
| SEQ ID NO: 30 | SEQ ID NO: 107 |
| SEQ ID NO: 30 | SEQ ID NO: 108 |
| SEQ ID NO: 31 | SEQ ID NO: 109 |
| SEQ ID NO: 31 | SEQ ID NO: 110 |
| SEQ ID NO: 31 | SEQ ID NO: 111 |
| SEQ ID NO: 32 | SEQ ID NO: 112 |
| SEQ ID NO: 33 | SEQ ID NO: 112 |
| SEQ ID NO: 34 | SEQ ID NO: 113 |
| SEQ ID NO: 34 | SEQ ID NO: 114 |
| SEQ ID NO: 34 | SEQ ID NO: 115 |
| SEQ ID NO: 35 | SEQ ID NO: 116 |
| SEQ ID NO: 35 | SEQ ID NO: 117 |
| SEQ ID NO: 35 | SEQ ID NO: 118 |
| SEQ ID NO: 36 | SEQ ID NO: 119 |
| SEQ ID NO: 36 | SEQ ID NO: 120 |
| SEQ ID NO: 36 | SEQ ID NO: 121 |
| SEQ ID NO: 37 | SEQ ID NO: 122 |
| SEQ ID NO: 37 | SEQ ID NO: 123 |
| SEQ ID NO: 38 | SEQ ID NO: 122; or |
| SEQ ID NO: 38 | SEQ ID NO: 123 |
| SEQ ID NO: 691 | SEQ ID NO: 692 |
| SEQ ID NO: 693 | SEQ ID NO: 694; or |
| SEQ ID NO: 695 | SEQ ID NO: 696 |
| SEQ ID NO: 247 | SEQ ID NO: 257 |
| SEQ ID NO: 247 | SEQ ID NO: 258 |
| SEQ ID NO: 248 | SEQ ID NO: 259 |
| SEQ ID NO: 249 | SEQ ID NO: 260 |
| SEQ ID NO: 249 | SEQ ID NO: 261 |
| SEQ ID NO: 250 | SEQ ID NO: 262 |
| SEQ ID NO: 250 | SEQ ID NO: 263 |
| SEQ ID NO: 250 | SEQ ID NO: 264 |
| SEQ ID NO: 251 | SEQ ID NO: 265 |
| SEQ ID NO: 251 | SEQ ID NO: 266 |
| SEQ ID NO: 251 | SEQ ID NO: 267 |
| SEQ ID NO: 252 | SEQ ID NO: 268 |
| SEQ ID NO: 252 | SEQ ID NO: 269 |
| SEQ ID NO: 253 | SEQ ID NO: 268 |
| SEQ ID NO: 253 | SEQ ID NO: 269 |
| SEQ ID NO: 253 | SEQ ID NO: 270 |
| SEQ ID NO: 254 | SEQ ID NO: 271 |
| SEQ ID NO: 254 | SEQ ID NO: 272 |
| SEQ ID NO: 255 | SEQ ID NO: 273 |
| SEQ ID NO: 255 | SEQ ID NO: 274 |
| SEQ ID NO: 255 | SEQ ID NO: 275 |
| SEQ ID NO: 256 | SEQ ID NO: 276; or |
| SEQ ID NO: 256 | SEQ ID NO: 277 |
| SEQ ID NO: 278 | SEQ ID NO: 295 |
| SEQ ID NO: 278 | SEQ ID NO: 296 |
| SEQ ID NO: 278 | SEQ ID NO: 297 |
| SEQ ID NO: 279 | SEQ ID NO: 295 |
| SEQ ID NO: 279 | SEQ ID NO: 296 |
| SEQ ID NO: 279 | SEQ ID NO: 297 |
| SEQ ID NO: 280 | SEQ ID NO: 298 |
| SEQ ID NO: 280 | SEQ ID NO: 299 |
| SEQ ID NO: 280 | SEQ ID NO: 300 |
| SEQ ID NO: 281 | SEQ ID NO: 298 |
| SEQ ID NO: 281 | SEQ ID NO: 299 |
| SEQ ID NO: 281 | SEQ ID NO: 300 |
| SEQ ID NO: 282 | SEQ ID NO: 301 |
| SEQ ID NO: 282 | SEQ ID NO: 302 |
| SEQ ID NO: 282 | SEQ ID NO: 303 |
| SEQ ID NO: 283 | SEQ ID NO: 301 |
| SEQ ID NO: 283 | SEQ ID NO: 302 |
| SEQ ID NO: 283 | SEQ ID NO: 303 |
| SEQ ID NO: 284 | SEQ ID NO: 304 |
| SEQ ID NO: 284 | SEQ ID NO: 305 |
| SEQ ID NO: 285 | SEQ ID NO: 307 |
| SEQ ID NO: 285 | SEQ ID NO: 308 |
| SEQ ID NO: 286 | SEQ ID NO: 306 |
| SEQ ID NO: 286 | SEQ ID NO: 307 |
| SEQ ID NO: 286 | SEQ ID NO: 308 |
| SEQ ID NO: 287 | SEQ ID NO: 306 |
| SEQ ID NO: 287 | SEQ ID NO: 307 |
| SEQ ID NO: 287 | SEQ ID NO: 308 |
| SEQ ID NO: 288 | SEQ ID NO: 309 |
| SEQ ID NO: 288 | SEQ ID NO: 310 |
| SEQ ID NO: 289 | SEQ ID NO: 309 |
| SEQ ID NO: 289 | SEQ ID NO: 310 |
| SEQ ID NO: 289 | SEQ ID NO: 311 |
| SEQ ID NO: 290 | SEQ ID NO: 312 |
| SEQ ID NO: 290 | SEQ ID NO: 313 |
| SEQ ID NO: 290 | SEQ ID NO: 314 |
| SEQ ID NO: 291 | SEQ ID NO: 315 |
| SEQ ID NO: 291 | SEQ ID NO: 316 |
| SEQ ID NO: 291 | SEQ ID NO: 317 |
| SEQ ID NO: 292 | SEQ ID NO: 318 |
| SEQ ID NO: 292 | SEQ ID NO: 319 |
| SEQ ID NO: 292 | SEQ ID NO: 320 |
| SEQ ID NO: 293 | SEQ ID NO: 318 |
| SEQ ID NO: 293 | SEQ ID NO: 319 |
| SEQ ID NO: 293 | SEQ ID NO: 320 |
| SEQ ID NO: 294 | SEQ ID NO: 321; or |
| SEQ ID NO: 294 | SEQ ID NO: 322 |
| SEQ ID NO: 323 | SEQ ID NO: 338 |
| SEQ ID NO: 323 | SEQ ID NO: 339 |
| SEQ ID NO: 324 | SEQ ID NO: 340 |
| SEQ ID NO: 324 | SEQ ID NO: 341 |
| SEQ ID NO: 325 | SEQ ID NO: 340 |
| SEQ ID NO: 325 | SEQ ID NO: 341 |
| SEQ ID NO: 325 | SEQ ID NO: 342 |
| SEQ ID NO: 326 | SEQ ID NO: 343 |
| SEQ ID NO: 326 | SEQ ID NO: 344 |
| SEQ ID NO: 326 | SEQ ID NO: 345 |
| SEQ ID NO: 327 | SEQ ID NO: 346 |
| SEQ ID NO: 327 | SEQ ID NO: 347 |
| SEQ ID NO: 328 | SEQ ID NO: 348 |
| SEQ ID NO: 328 | SEQ ID NO: 349 |
| SEQ ID NO: 328 | SEQ ID NO: 350 |
| SEQ ID NO: 329 | SEQ ID NO: 351 |
| SEQ ID NO: 329 | SEQ ID NO: 352 |
| SEQ ID NO: 329 | SEQ ID NO: 353 |
| SEQ ID NO: 329 | SEQ ID NO: 354 |
| SEQ ID NO: 330 | SEQ ID NO: 351 |
| SEQ ID NO: 330 | SEQ ID NO: 352 |
| SEQ ID NO: 330 | SEQ ID NO: 353 |
| SEQ ID NO: 330 | SEQ ID NO: 354 |
| SEQ ID NO: 331 | SEQ ID NO: 355 |
| SEQ ID NO: 331 | SEQ ID NO: 356 |
| SEQ ID NO: 332 | SEQ ID NO: 357 |
| SEQ ID NO: 332 | SEQ ID NO: 358 |
| SEQ ID NO: 332 | SEQ ID NO: 359 |
| SEQ ID NO: 333 | SEQ ID NO: 357 |
| SEQ ID NO: 333 | SEQ ID NO: 358 |
| SEQ ID NO: 333 | SEQ ID NO: 359 |
| SEQ ID NO: 334 | SEQ ID NO: 357 |
| SEQ ID NO: 334 | SEQ ID NO: 358 |
| SEQ ID NO: 334 | SEQ ID NO: 359 |
| SEQ ID NO: 335 | SEQ ID NO: 360 |
| SEQ ID NO: 336 | SEQ ID NO: 361 |
| SEQ ID NO: 336 | SEQ ID NO: 362 |
| SEQ ID NO: 336 | SEQ ID NO: 363 |
| SEQ ID NO: 337 | SEQ ID NO: 361 |
| SEQ ID NO: 337 | SEQ ID NO: 362; or |
| SEQ ID NO: 337 | SEQ ID NO: 363 |
| SEQ ID NO: 364 | SEQ ID NO: 397 |
| SEQ ID NO: 364 | SEQ ID NO: 398 |
| SEQ ID NO: 365 | SEQ ID NO: 399 |
| SEQ ID NO: 365 | SEQ ID NO: 400 |
| SEQ ID NO: 365 | SEQ ID NO: 401 |
| SEQ ID NO: 366 | SEQ ID NO: 402 |
| SEQ ID NO: 366 | SEQ ID NO: 403 |
| SEQ ID NO: 366 | SEQ ID NO: 404 |
| SEQ ID NO: 367 | SEQ ID NO: 405 |
| SEQ ID NO: 367 | SEQ ID NO: 406 |
| SEQ ID NO: 367 | SEQ ID NO: 407 |
| SEQ ID NO: 368 | SEQ ID NO: 408 |
| SEQ ID NO: 368 | SEQ ID NO: 409 |
| SEQ ID NO: 369 | SEQ ID NO: 410 |
| SEQ ID NO: 370 | SEQ ID NO: 411 |
| SEQ ID NO: 371 | SEQ ID NO: 412 |
| SEQ ID NO: 371 | SEQ ID NO: 413 |
| SEQ ID NO: 371 | SEQ ID NO: 414 |
| SEQ ID NO: 372 | SEQ ID NO: 415 |
| SEQ ID NO: 372 | SEQ ID NO: 416 |
| SEQ ID NO: 373 | SEQ ID NO: 417 |
| SEQ ID NO: 373 | SEQ ID NO: 418 |
| SEQ ID NO: 373 | SEQ ID NO: 419 |
| SEQ ID NO: 374 | SEQ ID NO: 417 |
| SEQ ID NO: 374 | SEQ ID NO: 418 |
| SEQ ID NO: 374 | SEQ ID NO: 420 |
| SEQ ID NO: 375 | SEQ ID NO: 421 |
| SEQ ID NO: 375 | SEQ ID NO: 422 |
| SEQ ID NO: 376 | SEQ ID NO: 423 |
| SEQ ID NO: 376 | SEQ ID NO: 424 |
| SEQ ID NO: 376 | SEQ ID NO: 425 |
| SEQ ID NO: 377 | SEQ ID NO: 426 |
| SEQ ID NO: 377 | SEQ ID NO: 427 |
| SEQ ID NO: 377 | SEQ ID NO: 428 |
| SEQ ID NO: 378 | SEQ ID NO: 429 |
| SEQ ID NO: 379 | SEQ ID NO: 430 |
| SEQ ID NO: 379 | SEQ ID NO: 431 |
| SEQ ID NO: 380 | SEQ ID NO: 432 |
| SEQ ID NO: 380 | SEQ ID NO: 433 |
| SEQ ID NO: 381 | SEQ ID NO: 434 |
| SEQ ID NO: 381 | SEQ ID NO: 435 |
| SEQ ID NO: 381 | SEQ ID NO: 436 |
| SEQ ID NO: 382 | SEQ ID NO: 437 |
| SEQ ID NO: 382 | SEQ ID NO: 438 |
| SEQ ID NO: 382 | SEQ ID NO: 439 |
| SEQ ID NO: 383 | SEQ ID NO: 440 |
| SEQ ID NO: 383 | SEQ ID NO: 441 |
| SEQ ID NO: 383 | SEQ ID NO: 442 |
| SEQ ID NO: 384 | SEQ ID NO: 443 |
| SEQ ID NO: 384 | SEQ ID NO: 444 |
| SEQ ID NO: 384 | SEQ ID NO: 445 |
| SEQ ID NO: 385 | SEQ ID NO: 446 |
| SEQ ID NO: 385 | SEQ ID NO: 447 |
| SEQ ID NO: 386 | SEQ ID NO: 448 |
| SEQ ID NO: 386 | SEQ ID NO: 449 |
| SEQ ID NO: 386 | SEQ ID NO: 450 |
| SEQ ID NO: 387 | SEQ ID NO: 451 |
| SEQ ID NO: 387 | SEQ ID NO: 452 |
| SEQ ID NO: 387 | SEQ ID NO: 453 |
| SEQ ID NO: 388 | SEQ ID NO: 451 |
| SEQ ID NO: 388 | SEQ ID NO: 454 |
| SEQ ID NO: 388 | SEQ ID NO: 455 |
| SEQ ID NO: 389 | SEQ ID NO: 454 |
| SEQ ID NO: 389 | SEQ ID NO: 455 |
| SEQ ID NO: 389 | SEQ ID NO: 456 |
| SEQ ID NO: 390 | SEQ ID NO: 454 |
| SEQ ID NO: 390 | SEQ ID NO: 455 |
| SEQ ID NO: 390 | SEQ ID NO: 456 |
| SEQ ID NO: 391 | SEQ ID NO: 457 |
| SEQ ID NO: 391 | SEQ ID NO: 458 |
| SEQ ID NO: 391 | SEQ ID NO: 459 |
| SEQ ID NO: 392 | SEQ ID NO: 460 |
| SEQ ID NO: 392 | SEQ ID NO: 461 |
| SEQ ID NO: 392 | SEQ ID NO: 462 |
| SEQ ID NO: 393 | SEQ ID NO: 463 |
| SEQ ID NO: 393 | SEQ ID NO: 464 |
| SEQ ID NO: 393 | SEQ ID NO: 465 |
| SEQ ID NO: 394 | SEQ ID NO: 466 |
| SEQ ID NO: 394 | SEQ ID NO: 467 |
| SEQ ID NO: 394 | SEQ ID NO: 468 |
| SEQ ID NO: 395 | SEQ ID NO: 469 |
| SEQ ID NO: 395 | SEQ ID NO: 470 |
| SEQ ID NO: 396 | SEQ ID NO: 469 |
| SEQ ID NO: 396 | SEQ ID NO: 470; or |
| SEQ ID NO: 396 | SEQ ID NO: 471 |
| SEQ ID NO: 472 | SEQ ID NO: 483 |
| SEQ ID NO: 472 | SEQ ID NO: 484 |
| SEQ ID NO: 472 | SEQ ID NO: 485 |
| SEQ ID NO: 473 | SEQ ID NO: 486 |
| SEQ ID NO: 473 | SEQ ID NO: 487 |
| SEQ ID NO: 473 | SEQ ID NO: 488 |
| SEQ ID NO: 474 | SEQ ID NO: 486 |
| SEQ ID NO: 474 | SEQ ID NO: 487 |
| SEQ ID NO: 474 | SEQ ID NO: 488 |
| SEQ ID NO: 475 | SEQ ID NO: 489 |
| SEQ ID NO: 475 | SEQ ID NO: 490 |
| SEQ ID NO: 475 | SEQ ID NO: 491 |
| SEQ ID NO: 476 | SEQ ID NO: 492 |
| SEQ ID NO: 476 | SEQ ID NO: 493 |
| SEQ ID NO: 477 | SEQ ID NO: 492 |
| SEQ ID NO: 477 | SEQ ID NO: 493 |
| SEQ ID NO: 478 | SEQ ID NO: 494 |
| SEQ ID NO: 478 | SEQ ID NO: 495 |
| SEQ ID NO: 478 | SEQ ID NO: 496 |
| SEQ ID NO: 479 | SEQ ID NO: 494 |
| SEQ ID NO: 479 | SEQ ID NO: 495 |
| SEQ ID NO: 479 | SEQ ID NO: 496 |
| SEQ ID NO: 480 | SEQ ID NO: 494 |
| SEQ ID NO: 480 | SEQ ID NO: 495 |
| SEQ ID NO: 480 | SEQ ID NO: 497 |
| SEQ ID NO: 481 | SEQ ID NO: 498 |
| SEQ ID NO: 481 | SEQ ID NO: 499 |
| SEQ ID NO: 481 | SEQ ID NO: 500 |
| SEQ ID NO: 482 | SEQ ID NO: 501 |
| SEQ ID NO: 482 | SEQ ID NO: 502; or |
| SEQ ID NO: 482 | SEQ ID NO: 503 |
| SEQ ID NO: 701 | SEQ ID NO: 702 |
| SEQ ID NO: 701 | SEQ ID NO: 703 |
| SEQ ID NO: 704 | SEQ ID NO: 705 |
| SEQ ID NO: 704 | SEQ ID NO: 706; or |
| SEQ ID NO: 707 | SEQ ID NO: 708 |
| SEQ ID NO: 504 | SEQ ID NO: 511 |
| SEQ ID NO: 504 | SEQ ID NO: 512 |
| SEQ ID NO: 505 | SEQ ID NO: 513 |
| SEQ ID NO: 505 | SEQ ID NO: 514 |
| SEQ ID NO: 505 | SEQ ID NO: 515 |
| SEQ ID NO: 505 | SEQ ID NO: 516 |
| SEQ ID NO: 506 | SEQ ID NO: 517 |
| SEQ ID NO: 506 | SEQ ID NO: 518 |
| SEQ ID NO: 507 | SEQ ID NO: 519 |
| SEQ ID NO: 507 | SEQ ID NO: 520 |
| SEQ ID NO: 507 | SEQ ID NO: 521 |
| SEQ ID NO: 507 | SEQ ID NO: 522 |
| SEQ ID NO: 508 | SEQ ID NO: 523 |
| SEQ ID NO: 508 | SEQ ID NO: 524 |
| SEQ ID NO: 508 | SEQ ID NO: 525 |
| SEQ ID NO: 508 | SEQ ID NO: 526 |
| SEQ ID NO: 509 | SEQ ID NO: 527 |
| SEQ ID NO: 510 | SEQ ID NO: 528; or |
| SEQ ID NO: 510 | SEQ ID NO: 529 |
| SEQ ID NO: 709 | SEQ ID NO: 710 |
| SEQ ID NO: 709 | SEQ ID NO: 711 |
| SEQ ID NO: 712 | SEQ ID NO: 713; or |
| SEQ ID NO: 712 | SEQ ID NO: 714 |
| SEQ ID NO: 530 | SEQ ID NO: 542 |
| SEQ ID NO: 530 | SEQ ID NO: 543 |
| SEQ ID NO: 530 | SEQ ID NO: 544 |
| SEQ ID NO: 531 | SEQ ID NO: 545 |
| SEQ ID NO: 531 | SEQ ID NO: 546 |
| SEQ ID NO: 532 | SEQ ID NO: 547 |
| SEQ ID NO: 532 | SEQ ID NO: 548 |
| SEQ ID NO: 532 | SEQ ID NO: 549 |
| SEQ ID NO: 533 | SEQ ID NO: 550 |
| SEQ ID NO: 534 | SEQ ID NO: 551 |
| SEQ ID NO: 534 | SEQ ID NO: 552 |
| SEQ ID NO: 534 | SEQ ID NO: 553 |
| SEQ ID NO: 535 | SEQ ID NO: 554 |
| SEQ ID NO: 535 | SEQ ID NO: 555 |
| SEQ ID NO: 535 | SEQ ID NO: 556 |
| SEQ ID NO: 536 | SEQ ID NO: 557 |
| SEQ ID NO: 536 | SEQ ID NO: 558 |
| SEQ ID NO: 537 | SEQ ID NO: 559 |
| SEQ ID NO: 537 | SEQ ID NO: 560 |
| SEQ ID NO: 537 | SEQ ID NO: 561 |
| SEQ ID NO: 538 | SEQ ID NO: 562 |
| SEQ ID NO: 538 | SEQ ID NO: 563 |
| SEQ ID NO: 539 | SEQ ID NO: 562 |
| SEQ ID NO: 539 | SEQ ID NO: 563 |
| SEQ ID NO: 540 | SEQ ID NO: 564; or |
| SEQ ID NO: 541 | SEQ ID NO: 564 |
| SEQ ID NO: 715 | SEQ ID NO: 716; or |
| SEQ ID NO: 717 | SEQ ID NO: 718 |
| SEQ ID NO: 565 | SEQ ID NO: 576 |
| SEQ ID NO: 565 | SEQ ID NO: 577 |
| SEQ ID NO: 565 | SEQ ID NO: 578 |
| SEQ ID NO: 565 | SEQ ID NO: 579 |
| SEQ ID NO: 566 | SEQ ID NO: 580 |
| SEQ ID NO: 566 | SEQ ID NO: 581 |
| SEQ ID NO: 566 | SEQ ID NO: 582 |
| SEQ ID NO: 566 | SEQ ID NO: 583 |
| SEQ ID NO: 567 | SEQ ID NO: 584 |
| SEQ ID NO: 567 | SEQ ID NO: 585 |
| SEQ ID NO: 567 | SEQ ID NO: 586 |
| SEQ ID NO: 567 | SEQ ID NO: 587 |
| SEQ ID NO: 568 | SEQ ID NO: 588 |
| SEQ ID NO: 568 | SEQ ID NO: 589 |
| SEQ ID NO: 568 | SEQ ID NO: 590 |
| SEQ ID NO: 569 | SEQ ID NO: 591 |
| SEQ ID NO: 569 | SEQ ID NO: 592 |
| SEQ ID NO: 569 | SEQ ID NO: 593 |
| SEQ ID NO: 570 | SEQ ID NO: 591 |
| SEQ ID NO: 570 | SEQ ID NO: 594 |
| SEQ ID NO: 571 | SEQ ID NO: 595 |
| SEQ ID NO: 571 | SEQ ID NO: 596 |
| SEQ ID NO: 571 | SEQ ID NO: 597 |
| SEQ ID NO: 571 | SEQ ID NO: 598 |
| SEQ ID NO: 572 | SEQ ID NO: 599 |
| SEQ ID NO: 572 | SEQ ID NO: 600 |
| SEQ ID NO: 573 | SEQ ID NO: 599 |
| SEQ ID NO: 573 | SEQ ID NO: 600 |
| SEQ ID NO: 573 | SEQ ID NO: 601 |
| SEQ ID NO: 574 | SEQ ID NO: 602 |
| SEQ ID NO: 574 | SEQ ID NO: 603 |
| SEQ ID NO: 574 | SEQ ID NO: 604 |
| SEQ ID NO: 574 | SEQ ID NO: 605 |
| SEQ ID NO: 575 | SEQ ID NO: 606 |
| SEQ ID NO: 575 | SEQ ID NO: 607; or |
| SEQ ID NO: 575 | SEQ ID NO: 608 |
| SEQ ID NO. 609 | SEQ ID NO. 619 |
| SEQ ID NO. 609 | SEQ ID NO. 620 |
| SEQ ID NO. 610 | SEQ ID NO. 621 |
| SEQ ID NO. 610 | SEQ ID NO. 622 |
| SEQ ID NO. 610 | SEQ ID NO. 623 |
| SEQ ID NO. 611 | SEQ ID NO. 624 |
| SEQ ID NO. 612 | SEQ ID NO. 625 |
| SEQ ID NO. 612 | SEQ ID NO. 626 |
| SEQ ID NO. 613 | SEQ ID NO. 627 |
| SEQ ID NO. 614 | SEQ ID NO. 628 |
| SEQ ID NO. 615 | SEQ ID NO. 629 |
| SEQ ID NO. 615 | SEQ ID NO. 630 |
| SEQ ID NO. 615 | SEQ ID NO. 631 |
| SEQ ID NO. 616 | SEQ ID NO. 632 |
| SEQ ID NO. 616 | SEQ ID NO. 633 |
| SEQ ID NO. 617 | SEQ ID NO. 634 |
| SEQ ID NO. 617 | SEQ ID NO. 635; or |
| SEQ ID NO. 618 | SEQ ID NO. 636 |
| SEQ ID NO: 723 | SEQ ID NO: 724; or |
| SEQ ID NO: 725 | SEQ ID NO: 726 |
| SEQ ID NO. 637 | SEQ ID NO. 642 |
| SEQ ID NO. 637 | SEQ ID NO. 643 |
| SEQ ID NO. 638 | SEQ ID NO. 644 |
| SEQ ID NO. 638 | SEQ ID NO. 645 |
| SEQ ID NO. 639 | SEQ ID NO. 646 |
| SEQ ID NO. 639 | SEQ ID NO. 647 |
| SEQ ID NO. 640 | SEQ ID NO. 648 |
| SEQ ID NO. 640 | SEQ ID NO. 649; or |
| SEQ ID NO. 641 | SEQ ID NO. 650 |
| SEQ ID NO: 727 | SEQ ID NO: 728; or |
| SEQ ID NO: 729 | SEQ ID NO: 730 |
| SEQ ID NO. 651 | SEQ ID NO. 660 |
| SEQ ID NO. 651 | SEQ ID NO. 661 |
| SEQ ID NO. 651 | SEQ ID NO. 662 |
| SEQ ID NO. 652 | SEQ ID NO. 663 |
| SEQ ID NO. 652 | SEQ ID NO. 664 |
| SEQ ID NO. 652 | SEQ ID NO. 665 |
| SEQ ID NO. 653 | SEQ ID NO. 666 |
| SEQ ID NO. 654 | SEQ ID NO. 667 |
| SEQ ID NO. 655 | SEQ ID NO. 668 |
| SEQ ID NO. 655 | SEQ ID NO. 669 |
| SEQ ID NO. 655 | SEQ ID NO. 670 |
| SEQ ID NO. 656 | SEQ ID NO. 671 |
| SEQ ID NO. 657 | SEQ ID NO. 672 |
| SEQ ID NO. 657 | SEQ ID NO. 673 |
| SEQ ID NO. 658 | SEQ ID NO. 674 |
| SEQ ID NO. 658 | SEQ ID NO. 675 |
| SEQ ID NO. 659 | SEQ ID NO. 676 |
| SEQ ID NO. 659 | SEQ ID NO. 677; or |
| SEQ ID NO. 659 | SEQ ID NO. 678 |
| SEQ ID NO: 719 | SEQ ID NO: 720; or |
| SEQ ID NO: 721 | SEQ ID NO: 722 |

119. The gene editing system of claim 1 or claim 118, comprising:
a. the hgRNA comprising a first CRISPR motif, the hgRNA spacer, and a first protein-binding motif, or a DNA polynucleotide encoding the hgRNA,
b. the mgRNA comprising a second CRISPR motif and the mgRNA spacer, or a DNA polynucleotide encoding the mgRNA,
c. a first CRISPR-associated protein (Cas protein), or a polynucleotide encoding the first Cas protein, wherein the first Cas protein binds to the first CRISPR motif,
d. a second Cas protein, or a polynucleotide encoding the second Cas protein, wherein the second Cas protein binds to the second CRISPR motif,
e. a first fusion protein comprising a nucleobase deaminase or a catalytic domain thereof and a first RNA binding domain, or a polynucleotide encoding the first fusion protein, wherein the nucleobase deaminase or the catalytic domain thereof and the first RNA binding domain are optionally connected by a linker, and wherein the first RNA binding domain binds to the first protein-binding motif,
wherein the first Cas protein and second Cas protein are the same or different.

120. The gene editing system of claim 119, further comprising
a. a protease, or a polynucleotide encoding the protease, and
b. a nucleobase deaminase inhibitor domain,
wherein the nucleobase deaminase inhibitor domain is connected to the nucleobase deaminase or the catalytic domain thereof in the first fusion protein optionally by a linker, and wherein there is a cleavage site for the protease between the nucleobase deaminase inhibitor domain and the nucleobase deaminase or the catalytic domain thereof.

121. The gene editing system of claim 120, further comprising
a second fusion protein comprising the protease and a second RNA binding domain, or a polynucleotide encoding the second fusion protein,
wherein the protease and the second RNA binding domain are optionally connected by a linker,
wherein the mgRNA further comprises a second protein-binding motif,
and wherein the second RNA binding domain binds to the second protein-binding motif.

122. The gene editing system of claim 120, wherein the protease is split into a first protease fragment and a second protease fragment, wherein the first and/or second protease fragment alone is not able to cleave the cleavage site.

123. The gene editing system of claim 122, further comprising
a. a second fusion protein comprising the first protease fragment and a second RNA binding domain, or a polynucleotide encoding the second fusion protein, wherein the first protease fragment and the second RNA binding domain are optionally connected by a linker, and
b. a third fusion protein comprising the second protease fragment and a third RNA binding domain, or a polynucleotide encoding the third fusion protein, wherein the second protease fragment and the third RNA binding domain are optionally connected by a linker,
wherein the mgRNA further comprises a second protein-binding motif and a third protein-binding motif,
wherein the second RNA binding domain binds to the second protein-binding motif, and
wherein the third RNA binding domain binds to the third protein-binding motif.

124. The gene editing system of claim 123, wherein the second and third RNA binding domains are the same or different, and the second and third protein-binding motifs are the same or different.

125. The gene editing system of claim 122, further comprising:
a second fusion protein comprising the first protease fragment and a second RNA binding domain, or a polynucleotide encoding the second fusion protein,
wherein the first protease fragment and the second RNA binding domain are optionally connected by a linker,
wherein the mgRNA further comprises a second protein-binding motif, and
wherein the second RNA binding domain binds to the second protein-binding motif.

126. The gene editing system of any of claims 120-125, wherein the protease is a TEV protease comprising a sequence of SEQ ID NO: 124.

127. The gene editing system of claim 126, wherein the first TEV protease fragment comprises a sequence of SEQ ID NO: 125.
